(19) United States
(12) Patent Application Publication
(10) Pub. No.: US 2025/0179454 A1
(43) Pub. Date: Jun. 5, 2025

(54) FUSION PROTEINS AND USES THEREOF FOR PRECISION EDITING

(71) Applicant: Mammoth Biosciences, Inc., Brisbane, CA (US)

(72) Inventors: Benjamin Julius RAUCH, San Francisco, CA (US); Yuchen GAO, South San Francisco, CA (US); William Douglass WRIGHT, Fairfield, CA (US); Stepan TYMOSHENKO, Sacramento, CA (US); Rohan GROVER, Greenbelt, MD (US); David PAEZ-ESPINO, Concord, CA (US)

(73) Assignee: Mammoth Biosciences, Inc., Brisbane, CA (US)

(21) Appl. No.: 19/044,826

(22) Filed: Feb. 4, 2025

Related U.S. Application Data
(63) Continuation of application No. PCT/US2023/072434, filed on Aug. 18, 2023.
(60) Provisional application No. 63/514,383, filed on Jul. 19, 2023.
(60) Provisional application No. 63/503,188, filed on May 19, 2023.
(60) Provisional application No. 63/485,663, filed on Feb. 17, 2023.
(60) Provisional application No. 63/481,716, filed on Jan. 26, 2023.
(60) Provisional application No. 63/382,545, filed on Nov. 7, 2022.
(60) Provisional application No. 63/380,673, filed on Oct. 24, 2022.
(60) Provisional application No. 63/371,979, filed on Aug. 19, 2022.

(51) Int. Cl. C12N 9/22; C12N 9/12; C12N 15/11; C12N 15/86; C12N 15/88

(52) U.S. Cl. CPC C12N 9/22; C12N 9/1276; C12N 15/111; C12N 15/86; C12N 15/88; C07K 2319/09; C12N 2310/20; C12N 2750/14143; C12Y 207/07049

(57) ABSTRACT

Disclosed herein are composition and methods of modifying a target double-stranded DNA (dsDNA) molecule in a cell. Systems, compositions, and methods may comprise a CRISPR-associated (Cas) protein, an RNA-dependent DNA polymerase, and/or one or more guide nucleic acids or uses thereof.

### CROSS-REFERENCE TO RELATED APPLICATIONS

The present application is a continuation of International PCT Application No. PCT/US2023/072434, filed Aug. 18, 2023, which claims priority to U.S. Provisional Application 63/371,979, filed Aug. 19, 2022; U.S. Provisional Application 63/380,673, filed Oct. 24, 2022; U.S. Provisional Application 63/382,545, filed Nov. 7, 2022; U.S. Provisional Application 63/481,716, filed Jan. 26, 2023; U.S. Provisional Application 63/485,663, filed Feb. 17, 2023; U.S. Provisional Application 63/503,188, filed May 19, 2023; and U.S. Provisional Application 63/514,383, filed Jul. 19, 2023, each of which are incorporated herein by reference in their entireties.

### REFERENCE TO THE ELECTRONIC SEQUENCE FILE

The contents of the electronic sequence listing (MABI_024_07US_SeqList_ST26.xml; Size: 673,878 bytes; and Date of Creation: Jan. 27, 2025) are herein incorporated by reference in its entirety.

### BACKGROUND

While gene disruption with CRISPR is now a mature technique, precision editing of a or few nucleotides in the human genome remains a major challenge. Therefore, there is a need for the development of systems that are capable of introducing specific nucleotide sequence modifications at a target site with high specificity and efficiency. However, there remains a need to increase their efficiency in installing the desired edits into the target sequence and/or removing deleterious off-target indel formation.

### SUMMARY

The present disclosure provides compositions and methods for precision editing of target nucleic acids. In some embodiments, the present disclosure provides systems and compositions that comprise an RNA-dependent DNA polymerase (RDDP), an effector protein (e.g., a CRISPR associated (Cas) protein), a guide RNA, and a template RNA. In some embodiments, the guide RNA and template RNA (rttRNA) are fused or linked as an extended guide RNA (e.g., rtgRNA). In some embodiments, the present disclosure further provides methods of modifying target nucleic acids utilizing the systems and compositions described herein.

In some embodiments, the present disclosure provides fusion proteins, systems, and methods for precision editing. In general, precision editing is an approach for gene editing using an effector protein (e.g., a Cas protein), a polymerase (e.g., an RDDP), and a template RNA with a desired edit to introduce specific gene edits into a target sequence.

Precision editing systems utilizing reverse transcriptases have been previously described (See e.g., Anzalone et al., Nature, Vol 576, 2019, p. 149-157). However, commonly used reverse transcriptases (e.g., reverse transcriptase from M-MLV) are large enzymes (around 650 amino acids), and the size of these enzymes presents a challenge for insertion of coding sequences for the effector protein and reverse transcriptase into the same delivery vector. Split editing systems have been described, wherein the effector protein and the reverse transcriptase are inserted into different vectors (Gao et al., Molecular Therapy, Volume 30, Issue 9, 2022, Pages 2942-2951; Liu et al., Nat Biotechnol 40, 1388-1393 (2022). However, these systems present additional challenges for use as therapeutics in that multiple vectors must be formulated and delivered to a subject. Moreover, editing efficiencies with precision editing systems remain low. Thus, there remains a need in the art for efficient RDDPs of compact size for incorporation into precision editing systems.

The present disclosure overcomes these challenges and provides RDDPs and effector proteins suitable for use in precision editing systems. In many instances, RDDPs and effector proteins provided herein comprise less than 500 amino acids, thereby enabling the combination of their coding sequences into a single delivery vector (either as two separate proteins or as a fusion protein). Furthermore, in some embodiments, the RDDPs provided herein demonstrate enhanced editing efficiencies compared to previously described enzymes and can therefore be used in various embodiments to enhance editing of target genes.

Compositions, systems, and methods disclosed herein may leverage nucleic acid modifying activities. Nucleic acid modifying activities may include cis cleavage activity, nicking activity, or nucleobase modifying activity. Compositions, systems and methods disclosed herein may be useful for modifying a single nucleotide of a target nucleic acid. Accordingly, compositions, systems and methods disclosed herein may be useful for correcting a sequence comprising a single nucleotide polymorphism (SNP) mutation to a wildtype sequence. Such gene editing may be referred to as “precision editing.” In some instances, compositions, systems, and methods are useful for the treatment of a disease or disorder. The disease or disorder may be associated with one or more mutations in the target nucleic acid.

In some embodiments, the present disclosure provides a system comprising: (a) an effector protein or a nucleic acid encoding the effector protein; (b) an RNA-directed DNA polymerase (RDDP) or a nucleic acid encoding the RDDP; (c) a guide RNA or nucleic acid encoding the guide RNA, wherein the guide RNA comprises (i) a first region comprising a protein binding sequence, and (ii) a second region comprising a spacer sequence that hybridizes to a target sequence of a first strand of a double stranded DNA (dsDNA) target nucleic acid, wherein the first region is located 5′ of the second region; and (d) a template RNA (rttRNA) or nucleic acid encoding the rttRNA, wherein the rttRNA comprises (i) a primer binding sequence (PBS), and (ii) a template sequence that hybridizes to the target sequence of a second strand of the dsDNA target nucleic acid. In some embodiments, the guide RNA is linked to the rttRNA. In some embodiments, the template sequence is located 5′ of the PBS, optionally wherein the 3′ end of the PBS is linked to the 5′ end of the template sequence. In some embodiments, the rttRNA is circularized.

In some embodiments, the rttRNA comprises a protein localization sequence that can localize a protein to the rttRNA. In some embodiments, the protein localization sequence comprises an MS2 coat protein localization sequence.

In some embodiments, the RDDP not fused to the effector protein, optionally wherein the RDDP is fused to an MS2 coat protein. In some embodiments, the RDDP is fused to the effector protein.

In some embodiments, the template sequence comprises a difference of at least one nucleotide relative to an equal length portion of the target sequence.

In some embodiments, the effector protein is a Type V Cas protein. In some embodiments, the length of the effector protein is 400 to 800 linked amino acids.

In some embodiments, the effector protein comprises an amino acid sequence that is at least 65%, at least 70%, at least 75%, at least 80%, at least 85%, at least 90%, at least 95%, at least 97%, at least 98%, at least 99%, or 100% identical to a sequence in TABLE 1. In some embodiments, the effector protein comprises an amino acid sequence that is at least 65%, at least 70%, at least 75%, at least 80%, at least 85%, at least 90%, at least 95%, at least 97%, at least 98%, at least 99%, or 100% identical SEQ ID NO: 12. In some embodiments, the effector protein comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 346.

In some embodiments, the effector protein comprises at least one amino acid alteration relative to a relative amino acid sequence in TABLE 1 that results in reduced nuclease activity, nickase activity, increased nickase activity, or a combination thereof. In some embodiments, the effector protein is a nickase, or wherein the method is performed under conditions wherein the effector protein has nicking activity.

In some embodiments, the primer binding sequence is less than 20, less than 19, less than 18, less than 17, less than 16, less than 16, less than 15, less than 14, less than 13, less than 12, less than 11, less than 10, less than 9, less than 8, less than 7, less than 6, less than 5 nucleotides long, and at least 4 nucleotides long. In some embodiments, the template sequence is less than 35, less than 34, less than 33, less than 32, less than 31, less than 30, less than 29, less than 28, less than 27, less than 26, less than 25, less than 24, less than 23, less than 22, less than 21, less than 20, less than 19, less than 18 nucleotides long, and at least 8 nucleotides long.

In some embodiments, the RDDP comprises an amino acid sequence that is at least 65%, at least 70%, at least 75%, at least 80%, at least 85%, at least 90%, at least 95%, at least 97%, at least 98%, at least 99%, or 100% identical to any one of SEQ ID NOs: 485-530. In some embodiments, the RDDP comprises an amino acid sequence that is at least 65%, at least 70%, at least 75%, at least 80%, at least 85%, at least 90%, at least 95%, at least 97%, at least 98%, at least 99%, or 100% identical to any one of SEQ ID NOs: 496, 498, 504, 506, or 514.

In some embodiments, the system comprises expression vector, wherein the expression vector comprises any combination of: the nucleic acid encoding the effector protein; the nucleic acid encoding the RDDP; the nucleic acid encoding the guide RNA; and the nucleic acid encoding the rttRNA. In some embodiments, the expression vector is an adeno-associated viral (AAV) vector, optionally wherein the AAV vector is an scAAV vector.

In some embodiments, the system comprises a lipid or lipid nanoparticle.

In some embodiments, the nucleic acid encoding the effector protein or the nucleic acid encoding the RDDP comprises a messenger RNA.

In some embodiments, the system comprises a non-homologous end joining (NHEJ) inhibitor.

In some embodiments, the present disclosure provides a pharmaceutical composition comprising any one of the effector protein, RDDP, guide RNA, template RNA, and a nucleic acid encoding the same; and a pharmaceutically acceptable excipient. In some embodiments, the present disclosure provides a cell modified by a system described herein. In some embodiments, the present disclosure provides a cell comprising a system described herein. In some embodiments, the cell is a eukaryotic cell.

In some embodiments, the present disclosure provides a method of modifying a target nucleic acid, the method comprising contacting a target nucleic acid with a system described herein. In some embodiments, the cell is a eukaryotic cell.

In some embodiments, the present disclosure provides a method of modifying a target double-stranded DNA (dsDNA) molecule in a cell, the method comprising: contacting the target dsDNA with: (a) an effector protein wherein the effector protein comprises an effector protein or a nuclease domain thereof; (b) a guide RNA comprising (i) a first region comprising a protein binding sequence, and (ii) a second region comprising a spacer sequence that is complementary to a target sequence of a target strand of the target dsDNA molecule, thereby cleaving at least the non-target strand of the target dsDNA to generate a single-stranded portion of the non-target strand having a free 3′ end; (c) a template RNA comprising (i) a primer binding sequence that hybridizes to a primer sequence on the non-target strand of the target dsDNA molecule that is formed when the non-target strand is cleaved; (ii) a template sequence that is complementary to at least a portion of the target sequence of the non-target strand of the target dsDNA molecule with the exception of at least one nucleotide; (d) an RNA-directed DNA polymerase (RDDP) that polymerizes a new strand of DNA on the template RNA, wherein the new strand comprises a difference of at least one nucleotide relative to the target sequence of the target strand, thereby modifying the target dsDNA molecule, wherein the effector protein is not a Cas9 protein.

In some embodiments, the guide RNA is linked to the template RNA. In some embodiments, the template RNA is linked to the guide RNA at the 3′ end of the guide RNA. In some embodiments, the primer binding sequence is linked to the guide RNA at the 5′ end of the guide RNA. In some embodiments, the guide RNA and template RNA are not linked.

In some embodiments, the effector protein is linked to the RDDP. In some embodiments, the effector protein is not linked to the RDDP.

In some embodiments, the first region is 5′ of the second region. In some embodiments, template sequence is 5′ of the primer binding sequence. In some embodiments, the template sequence is 3′ of the primer binding sequence. In some embodiments, the template RNA includes a protein localization sequence that can localize a protein to the template RNA. In some embodiments, the protein localization sequence comprises an MS2 localization sequence. In some embodiments, the RDDP is linked to an MS2 coat protein.

In some embodiments, the method comprises contacting the target dsDNA with an integrase. In some embodiments, the new strand is incorporated into the target dsDNA molecule via the cell's native repair machinery.

In some embodiments, the difference of at least one nucleotide relative to the target sequence of the target strand is a nucleotide substitution selected from (1) G to T, (2) G to A, (3) G to C (4) T to G, (5) T to A, (6) T to C, (7) C to G, (8) C to T, (9) C to A, (10) A to T, (11) A to G, or (12) A to C. In some embodiments, the difference of at least one nucleotide relative to the target sequence of the target strand is an insertion or deletion of 1, 2, 3, 4, 5, 6, 7, 8, 9, 10, 11, 12, 13, 14, 15, 16, 17, 18, 19, 20, 21, 22, 23, 24, or 25 nucleotides.

In some embodiments, the effector protein is a Type V Cas protein. In some embodiments, the Type V Cas protein recognizes a protospacer adjacent motif (PAM) that is 5′ of the target sequence on the non-target strand. In some embodiments, the non-target strand of the target dsDNA is cleaved 10, 11, 12, 13, 14, 15, 16, 17, 18, 19, 20, 21, 22, 23, 24 or 25 nucleotides 3′ of the 3′ most nucleotide of the PAM. In some embodiments, the Type V Cas protein is a CasPhi protein. In some embodiments, the CasPhi protein is selected from CasPhi12, CasPhi18, CasPhi20, CasPhi22, CasPhi24, CasPhi25, CasPhi28, CasPhi30, CasPhi32, CasPhi33, CasPhi37, CasPhi39, CasPhi43, and CasPhi45.

In some embodiments, the length of the effector protein is less than 900, less than 800, less than 700, less than 600, less than 500 or less than 400 linked amino acids, and at least 350 linked amino acids. In some embodiments, the effector protein also nicks the target strand of the target dsDNA molecule. In some embodiments, the effector protein is a nickase, or wherein the method is performed under conditions wherein the effector protein has nicking activity.

In some embodiments, the template sequence includes a non-natural nucleotide sequence.

In some embodiments, the primer binding sequence is less than 20, less than 19, less than 18, less than 17, less than 16, less than 16, less than 15, less than 14, less than 13, less than 12, less than 11, less than 10, less than 9, less than 8, less than 7, less than 6, less than 5 nucleotides long, and at least 4 nucleotides long.

In some embodiments, the template sequence is less than 35, less than 34, less than 33, less than 32, less than 31, less than 30, less than 29, less than 28, less than 27, less than 26, less than 25, less than 24, less than 23, less than 22, less than 21, less than 20, less than 19, less than 18 nucleotides long, and at least 8 nucleotides long.

In some embodiments, the method comprises contacting the dsDNA with a non-homologous end joining (NHEJ) inhibitor.

In some embodiments, the effector protein comprises an amino acid sequence that is at least 90%, at least 95%, at least 98%, at least 99% or 100% identical to SEQ ID NO: 12. In some embodiments, the length of the spacer sequence is 15 nucleotides.

In some embodiments, the cell is a mammalian cell. In some embodiments, the cell is a human cell.

In some embodiments, the present disclosure provides a system for modifying a target double stranded DNA (dsDNA) comprising one or more nucleic acids, wherein the one or more nucleic acids comprise: (a) a first nucleotide sequence encoding an effector protein, or a nuclease domain thereof; (b) a second nucleotide sequence comprising a guide RNA or a DNA encoding the guide RNA wherein the guide RNA comprises a first region comprising a protein binding sequence and a second region comprising a spacer sequence that is complementary to a target sequence of a target strand of the target dsDNA molecule; (c) a third nucleotide sequence encoding a template RNA; and (d) a fourth nucleotide sequence encoding an RNA-directed DNA polymerase (RDDP); and wherein the effector protein is not a Cas9 protein.

In some embodiments, the one or more nucleic acids is an mRNA, and wherein the mRNA comprises the first sequence and/or the fourth sequence. In some embodiments, any one of the first, second, third, and fourth sequences are located on an expression vector. In some embodiments, the template RNA comprises a primer binding sequence and a template sequence. In some embodiments, the guide RNA is linked to the template RNA. In some embodiments, the template RNA is linked to the guide RNA at the 3′ end of the guide RNA. In some embodiments, the primer binding sequence is linked to the guide RNA at the 5′ end of the guide RNA.

In some embodiments, the effector protein is linked to the RDDP. In some embodiments, the effector protein is not linked to RDDP. In some embodiments, the first region is 5′ of the second region. In some embodiments, the template sequence is 5′ of the primer binding sequence. In some embodiments, the template sequence is 3′ of the primer binding sequence.

In some embodiments, the template RNA includes a protein localization sequence that can localize a protein to the template RNA. In some embodiments, the protein localization sequence comprises an MS2 localization sequence. In some embodiments, the RDDP is linked to an MS2 protein.

In some embodiments, the effector protein is a Type V Cas protein. In some embodiments, the Type V Cas protein is a CasPhi protein. In some embodiments, the CasPhi protein is selected from CasPhi12, CasPhi18, CasPhi20, CasPhi22, CasPhi24, CasPhi25, CasPhi28, CasPhi30, CasPhi32, CasPhi33, CasPhi37, CasPhi39, CasPhi43, and CasPhi45. In some embodiments, the length of the Type V Cas protein is less than 900, less than 800, less than 700, less than 600, less than 500 or less than 400 linked amino acids, and at least 350 linked amino acids.

In some embodiments, the system includes a fifth nucleotide sequence encoding an integrase. In some embodiments, the fifth nucleotide sequence is located on an expression vector.

In some embodiments, the present disclosure provides an isolated polynucleotide or polynucleotides comprising a nucleotide sequence described herein. In some embodiments, the present disclosure provides a vector comprising an isolated polynucleotide described herein.

In some embodiments, the present disclosure provides a recombinant AAV expression cassette comprising sequences encoding: (a) a first ITR and a first promoter; (b) a first nucleotide sequence encoding an effector protein and a guide RNA, or a nuclease domain thereof; (c) optionally a second promoter; (d) a second nucleotide sequence encoding a template RNA, wherein if there is no second promoter the template RNA is linked to the guide RNA at the 3′ end of the guide RNA; (c) optionally a third promoter; (f) a third nucleotide sequence encoding a RDDP; (g) a second ITR, wherein the AAV expression cassette is self-complementary; and wherein the effector protein is not Cas9.

In some embodiments, the effector protein is a Type V Cas protein. In some embodiments, the Type V Cas protein is a CasPhi protein. In some embodiments, the CasPhi protein is selected from CasPhi12, CasPhi18, CasPhi20, CasPhi22, CasPhi24, CasPhi25, CasPhi28, CasPhi30, CasPhi32, CasPhi33, CasPhi37, CasPhi39, CasPhi43, and CasPhi45.

In some embodiments, the AAV further includes a fourth nucleotide sequence encoding an integrase.

In some embodiments, the present disclosure provides a pharmaceutical composition comprising a recombinant AAV described herein and a pharmaceutical acceptable carrier. In some embodiments, the present disclosure provides a cell or a population of cells comprising a recombinant AAV or vector described herein.

In some embodiments, the present disclosure provides a system for modifying a target double stranded DNA (dsDNA) comprising one or more nucleic acids, wherein the one or more nucleic acids comprise: (a) a first nucleotide sequence encoding an effector protein, wherein the effector protein comprises an amino acid sequence that is at least 90%, at least 95%, at least 98%, at least 99% or 100% identical to SEQ ID NO: 12; (b) a second nucleotide sequence comprising a guide RNA or a DNA encoding the guide RNA wherein the guide RNA comprises a first region comprising a protein binding sequence and a second region comprising a spacer sequence that is complementary to a target sequence of a target strand of the target dsDNA molecule, wherein the length of the spacer sequence is 15 nucleotides; (c) a third nucleotide sequence encoding a template RNA; and (d) a fourth nucleotide sequence encoding an RNA-directed DNA polymerase (RDDP).

In some embodiments, the present disclosure provides a method of modifying a target double stranded DNA (dsDNA) comprising contacting the dsDNA with a system described herein. In some embodiments, the method comprises contacting a cell comprising the dsDNA. In some embodiments, the cell is a mammalian cell. In some embodiments, the cell is a human cell.

In some embodiments, the present disclosure provides a method of precision editing a double-stranded DNA in a cell to include a desired change, the method comprising contacting the double-stranded DNA with: (a) a Cas protein, or a nuclease domain thereof, that is at least 90% identical to SEQ ID NO: 346; (b) a guide RNA, comprising: (i) a first region that comprises a protein binding sequence, and (ii) a second region that comprises a spacer sequence that is complementary to a target sequence of a target strand of the target dsDNA molecule, thereby cleaving the non-target DNA strand of the double-stranded DNA at a location in relation to a PAM site to generate a free single-stranded DNA having a 3′ end; (c) a template RNA comprising: (i) a primer binding sequence that hybridizes to a primer sequence of the target dsDNA molecule that is formed when the non-target strand is cleaved; (ii) a template sequence that is complementary to at least a portion of the target sequence of the target dsDNA molecule with the exception of at least one nucleotide; (d) an RNA-directed DNA polymerase (RDDP) that polymerizes a new strand of DNA on the template RNA, wherein the new strand comprises a difference of at least one nucleotide relative to the appropriate Watson-Crick pairing, thereby introducing a modification into the target dsDNA molecule.

In some embodiments, the guide RNA is linked to the template RNA. In some embodiments, the template RNA is linked to the guide RNA at the 3′ end of the guide RNA. In some embodiments, the primer binding sequence is linked to the guide RNA at the 5′ end of the guide RNA. In some embodiments, the guide RNA and template RNA are not linked. In some embodiments, the effector protein is linked to the RDDP. In some embodiments, the effector protein is not linked to the RDDP. In some embodiments, the first region is 5′ of the second region. In some embodiments, the first region is 3′ of the second region. In some embodiments, the template sequence is 5′ of the primer binding sequence. In some embodiments, the template sequence is 3′ of the primer binding sequence.

In some embodiments, the template RNA includes a protein localization sequence that can localize a protein to the template RNA. In some embodiments, the protein localization sequence comprises an MS2 localization sequence. In some embodiments, the RDDP is linked to an MS2 protein. In some embodiments, the template RNA is bound to an MS2 aptamer.

In some embodiments, the Cas protein is 95% identical to SEQ ID NO: 346. In some embodiments, the Cas protein is 98% identical to SEQ ID NO: 346. In some embodiments, the Cas protein is 99% identical to SEQ ID NO: 346. In some embodiments, the Cas protein is 100% identical to SEQ ID NO: 346.

In some embodiments, the PAM sequence is selected from the group consisting of NNTN, TNTR or TNTG. In some embodiments, the cleavage of the non-target strand is between 10 and 35 nucleotides 3′ of the PAM sequence. In some embodiments, the cleavage of the non-target strand is between 20-30 nucleotides 3′ of the PAM sequence. In some embodiments, the cleavage of the non-target strand is 29 nucleotides 3′ of the PAM sequence.

In some embodiments, the primer binding sequence binds to the non-target strand. In some embodiments, the template sequence introduces a modification into the target strand. In some embodiments, the primer binding sequence binds to the target strand. In some embodiments, the template sequence introduces a modification into the non-target strand.

In some embodiments, the method comprises contacting a cell comprising the dsDNA. In some embodiments, the cell is a eukaryotic cell. In some embodiments, the cell is a mammalian cell. In some embodiments, the cell is a human cell.

In some embodiments, the method further includes a NHEJ inhibitor. In some embodiments, the inhibitor is AZD7648.

In some embodiments, the present disclosure provides a system for precision editing a target double stranded DNA (dsDNA) comprising one or more nucleic acids, wherein the one or more nucleic acids comprise: (a) a first nucleotide sequence encoding a Cas protein, or a nuclease domain thereof, that is at least 90% identical to SEQ ID NO: 346; (b) a second nucleotide sequence comprising a guide RNA or a DNA encoding the guide RNA wherein the guide RNA comprises a first region comprising a protein binding sequence and a second region comprising a spacer sequence that is complementary to a target sequence of the target dsDNA molecule; (c) a third nucleotide sequence encoding a template RNA, wherein the template RNA comprises a primer binding sequence and a template sequence; and (d) a fourth nucleotide sequence encoding an RNA-directed DNA polymerase (RDDP).

In some embodiments, the one or more nucleic acids is an mRNA, and wherein the mRNA comprises the first sequence and/or the fourth sequence. In some embodiments, the template RNA comprises a primer binding sequence and a template sequence. In some embodiments, the guide RNA is linked to the template RNA. In some embodiments, the template RNA is linked to the guide RNA at the 3′ end of the guide RNA. In some embodiments, the primer binding sequence is linked to the guide RNA at the 5′ end of the guide RNA. In some embodiments, the effector protein is linked to the RDDP. In some embodiments, the effector protein is not linked to RDDP. In some embodiments, the first region is 5′ of the second region. In some embodiments, the template sequence is 5′ of the primer binding sequence. In some embodiments, the template sequence is 3′ of the primer binding sequence.

In some embodiments, the Cas protein is 95% identical to SEQ ID NO: 346. In some embodiments, the Cas protein is 98% identical to SEQ ID NO: 346. In some embodiments, the Cas protein is 99% identical to SEQ ID NO: 346. In some embodiments, the Cas protein is 100% identical to SEQ ID NO: 346.

In some embodiments, the template RNA includes a protein localization sequence that can localize a protein to the template RNA. In some embodiments, the protein localization sequence comprises an MS2 localization sequence. In some embodiments, the RDDP is linked to an MS2 protein. In some embodiments, the template RNA is linked to an MS2 aptamer. In some embodiments, the MS2 aptamer has the sequence of SEQ ID NO: 420.

In some embodiments, the template RNA is linked to at least one ribozyme sequence. In some embodiments, the template RNA is linked to two ribozyme sequences. In some embodiments, the two ribozyme sequences are SEQ ID NO: 418 and 422. In some embodiments, the template RNA includes at least one homology arm to bind to the ribozyme sequence. In some embodiments, the template RNA includes two homology arms. In some embodiments, the two homology arm sequences are SEQ ID NO: 419 and 421. In some embodiments, the template RNA is oriented starting in the 5′ position with the 5′ ribozyme sequence of SEQ ID NO: 418; the 5′ homology arm sequence of SEQ ID NO: 419; the MS2 aptamer sequence of SEQ ID NO: 420; primer binding sequence and a template sequence selected from the SEQ ID NOs: 356-417; the 3′ homology arm sequence of SEQ ID NO: 421; and the 3′ ribozyme sequence of SEQ ID NO: 422. In some embodiments, the template RNA consists of any one of SEQ ID NOs: 423-484.

In some embodiments, the first region of the second nucleotide is 3′ of the second region of the second nucleotide. In some embodiments, the first region of the second nucleotide is 5′ of the second region of the second nucleotide. In some embodiments, the guide RNA further includes a handle sequence. In some embodiments, the handle sequence is SEQ ID NO: 348. In some embodiments, the protein binding sequence is SEQ ID NO: 347.

In some embodiments, any one of the first, second, third, and fourth sequences are located on an expression vector. In some embodiments, the expression vector is an AAV. In some embodiments, the AAV is a self-complementary AAV.

In some embodiments, the present disclosure provides an isolated polynucleotide or polynucleotides comprising a nucleotide sequence described herein. In some embodiments, the present disclosure provides a vector comprising an isolated polynucleotide described herein.

In some embodiments, the present disclosure provides a fusion protein comprising an effector protein and an RNA-dependent DNA polymerase (RDDP), wherein the RDDP comprises an amino acid sequence that is at least 90% or at least 95% identical to any one of SEQ ID NOs: 496, 498, 504, 506, or 514. In some embodiments, the RDDP comprises or consists of any one of SEQ ID NOs: 496, 498, 504, 506, or 514. In some embodiments, the length of the RDDP is less than 700 or less than 600 linked amino acids.

In some embodiments, the effector protein comprises a nickase activity.

In some embodiments, the effector protein comprises an amino acid sequence that is at least 90% or at least 95% identical to any one of SEQ ID NOs: 1-134 or 346. In some embodiments, the RDDP is fused to the N-terminus of the effector protein. In some embodiments, the RDDP is fused to the C-terminus of the effector protein.

In some embodiments, the present disclosure provides a polynucleotide encoding a fusion protein described herein. In some embodiments, the present disclosure provides a vector comprising a polynucleotide described herein. In some embodiments, the vector is a viral vector. In some embodiments, the viral vector is an adeno-associated virus (AAV) vector.

In some embodiments, the present disclosure provides a system comprising: (a) the fusion protein described herein, polynucleotide encoding the same, or a vector comprising the polynucleotide; and (b) an extended guide RNA (rtgRNA) comprising a guide RNA and a template RNA.

In some embodiments, the present disclosure provides a system comprising: (a) an RNA-dependent DNA polymerase (RDDP) protein comprising an amino acid sequence that is at least 90% or at least 95% identical to any one of SEQ ID NOs: 496, 498, 504, 506, or 514; (b) an effector protein; and (c) an extended guide RNA (rtgRNA) comprising a guide RNA and a template RNA.

In some embodiments, the effector protein comprises a nickase activity. In some embodiments, the guide RNA comprises a first region that is bound by the effector protein of the fusion protein, and a second region comprising a spacer sequence that is complementary to a target sequence of a target nucleic acid. In some embodiments, the target nucleic acid is a dsDNA. In some embodiments, the template RNA comprises a primer binding sequence and a template sequence. In some embodiments, the primer binding sequence hybridizes to a primer sequence of the target nucleic acid. In some embodiments, the template sequence is complementary to at least a portion of the target nucleic acid with the exception of at least one nucleotide. In some embodiments, the composition further comprises a second guide RNA. In some embodiments, the second guide RNA further enhances editing efficiency.

In some embodiments, the present disclosure provides a mammalian cell comprising an RNA-dependent DNA polymerase (RDDP) protein comprising an amino acid sequence that is at least 90% or at least 95% identical to any one of SEQ ID NOs: 496, 498, 504, 506, or 514. In some embodiments, the RDDP protein is fused to an effector protein.

In some embodiments, the present disclosure provides a mammalian cell comprising a system described herein. In some embodiments, the cell is a human cell or a murine cell.

In some embodiments, the present disclosure provides a method of editing a target nucleic acid sequence in a cell, comprising contacting a cell with a fusion protein described herein, a polynucleotide encoding the same, the vector comprising the polynucleotide, or a system described herein. In some embodiments, contacting comprises: (a) transfecting, optionally via lipofection or (b) transducing, optionally via AAV delivery. In some embodiments, the method results in an editing efficiency of greater than 1%.

### DESCRIPTION OF FIGURES

FIG. 1A shows the orientation of an effector protein binding to the target strand (T-strand, TS) and the relative positions of the RuvC sites to the PAM sequence.

FIG. 1B shows the position of the cleavage site on the nontarget strand (NT-strand, NTS) and/or the target strand for effector proteins relative to the PAM sequence.

FIG. 2 shows exemplary plasmids for compositions, systems and methods disclosed herein. In other embodiments, plasmids encode a guide RNA comprising a spacer sequence and a protein binding sequence wherein the guide RNA is separate from a primer binding sequence linked to a template sequence. In some embodiments, the plasmids encode an effector protein and a partner protein that are not fused, instead of a fusion protein.

FIG. 3 shows an exemplary gene editing system comprising an extended guide RNA with the primer binding sequence and template sequence located 5′ of a repeat sequence and a spacer sequence. The effector protein is fused to the RNA-directed DNA polymerase (RDDP). System components shown in FIG. 3 are exemplary and non-limiting. For instance, the 5′ extended rtgRNA may comprise additional nucleotides beyond those labeled as spacer, repeat, linker, PBS and RT template (RTT).

FIG. 4 shows an exemplary gene editing system comprising an extended guide RNA with the primer binding sequence and template sequence located 3′ of a repeat sequence and a spacer sequence. The effector protein is fused to the RNA-directed DNA polymerase (RDDP). System components shown in FIG. 4 are exemplary and non-limiting. For instance, the 5′ extended rtgRNA may comprise additional nucleotides beyond those labeled as spacer, repeat, linker, PBS and RT template (RTT).

FIGS. 5A-5B show exemplary split protein RNA design gene editing systems comprising a guide RNA with the repeat and spacer sequences separate from the primer binding sequence and template sequence. The effector protein is separate from the RDDP. The effector protein is localized to the target nucleic acid via the guide RNA where it can nick the target nucleic acid. The primer biding sequence and template binding sequence are linked to an MS2 aptamer. The RDDP is fused to an MS2 coat protein that is capable of binding the MS2 aptamer, thereby localizing the RDDP to the nicked DNA. System components shown in FIGS. 5A and 5B are exemplary and non-limiting. For instance, the gRNA and/or rttRNA may comprise additional nucleotides beyond those labeled as spacer, repeat, linker, PBS and RT template (RTT).

FIG. 6 shows a nickase cleavage assay that demonstrated nickase activity of CasPhi.12 in HEK293T cells.

FIG. 7 shows precision editing in HEK293T cells with CasPhi.12.

FIG. 8 shows precision editing in HEK293T cells with CasPhi. 12 is enhanced by a non-homologous end joining (NHEJ) inhibitor.

FIG. 9 shows an overview of a system comprising CasM.265466 for precise editing. CasM.265466 generates a double-stranded break on target DNA. A rttRNA hybridizes with the non-target strand (as shown) or target strand (not shown) via complementarity between the PBS region and the target DNA. An RDDP is recruited to the target site by binding of a fused MS2 coat protein to the MS2 aptamer of the rttRNA. The RNA/DNA hybrid serves as a binding substrate for the RDDP, which then synthesizes new cDNA along the exposed 3′ end of the target DNA using the RTT as a reverse transcription template. Edits encoded in the RTT are thereby written into the genome.

FIG. 10A and FIG. 10B show how CasM.265466 is predicted to edit the non-target strand (NTS) and the target strand (TS) of a double stranded DNA (dsDNA) target nucleic acid. FIG. 10A shows NTS editing. FIG. 10B shows TS editing.

FIGS. 11A-11C shows data generated with a precise editing system that comprises CasM.265466. These data show that this system can generate precise edits in a NTS of a dsDNA target nucleic acid. FIG. 11A shows precise editing in the absence of an NHEJ inhibitor (NHEJi). FIG. 11B shows precise editing in the presence of an NHEJ inhibitor (NHEJi). FIG. 11C shows exemplary next generation sequencing (NGS) data, with the precise edit(s) highlighted.

FIG. 12 shows data generated with a precise editing system that comprises CasM.265466. These data show that this system can generate precise edits in a TS of a dsDNA target nucleic acid. FIG. 12A shows precise editing in the absence of an NHEJ inhibitor (NHEJi). FIG. 12B shows precise editing in the presence of an NHEJ inhibitor (NHEJi). FIG. 12C shows exemplary NGS data, with the precise edit(s) highlighted.

FIG. 13 shows an example of a split protein/RNA system for precise editing with Type II Cas effectors (e.g., Cas9), wherein the rttRNA is circularized.

FIG. 14 shows an example of a split protein/RNA system for precise editing with Type V Cas effectors, wherein the rttRNA is circularized. CasPhi (e.g., CasPhi. 12 disclosed herein) is a non-limiting example of a Type V Cas effector.

FIG. 15 illustrates the editing level of five RDDP candidates and the wild-type M-MLV RDDP against different targets in HEK293T cells.

FIG. 16A-FIG. 16B illustrates the editing levels of RDDP candidates and MMLV RDDP against gene target sites. FIG. 16A shows editing levels in HEK target sites. FIG. 16B shows editing levels in FANCF target sites.

FIG. 17 illustrates editing levels of variant RDDP candidates comprising arginine and lysine mutations in FANCF (top panel) and HEK (bottom panel) target sites.

### DETAILED DESCRIPTION

It is to be understood that both the foregoing general description and the following detailed description are exemplary, and explanatory only, and are not restrictive of the disclosure.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

### Definitions

Unless otherwise indicated, all technical terms used herein have the same meaning as commonly understood by one of ordinary skill in the art. Unless otherwise indicated or obvious from context, the following terms have the following meanings:

As used in this specification and the appended claims, the singular forms “a,” “an,” and “the” include plural references unless the context clearly dictates otherwise.

Any reference to “or” herein is intended to encompass “and/or” unless otherwise stated. As used herein, the term “and/or” includes any and all combinations of one or more of the associated listed items.

Use of the term “including” as well as other forms, such as “includes” and “included,” is not limiting.

As used herein, the term “comprise” and its grammatical equivalents specifies the presence of stated features, integers, steps, operations, elements, and/or components, but does not preclude the presence or addition of one or more other features, integers, steps, operations, elements, components, and/or groups thereof.

As used herein, the term “about” in reference to a number or range of numbers is understood to mean the stated number and numbers +/−10% thereof, or 10% below the lower listed limit and 10% above the higher listed limit for the values listed for a range.

The terms “% identical,” “% identity,” and “percent identity,” or grammatical equivalents thereof, with reference to an amino acid sequence or nucleotide sequence, refer to the percent of residues that are identical between respective positions of two sequences when the two sequences are aligned for maximum sequence identity. The % identity is calculated by dividing the total number of the aligned residues by the number of the residues that are identical between the respective positions of the at least two sequences and multiplying by 100. Generally, computer programs can be employed for such calculations. Illustrative programs that compare and align pairs of sequences, include ALIGN (Myers and Miller, Comput Appl Biosci. 1988 March; 4 (1): 11-7), FASTA (Pearson and Lipman, Proc Natl Acad Sci USA. 1988 April; 85 (8): 2444-8; Pearson, Methods Enzymol. 1990; 183:63-98) and gapped BLAST (Altschul et al., Nucleic Acids Res. 1997 Sep. 1; 25 (17): 3389-40), BLASTP, BLASTN, or GCG (Devereux et al., Nucleic Acids Res. 1984 Jan. 11; 12 (1 Pt 1): 387-95). To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, cbi.ac.uk/Tools/msa/tcoffee/, ebi.ac.uk/Tools/msa/muscle/, mafft.cbrc.jp/alignment/software/. See, e.g., Altschul et al. (1990), J. Mol. Bioi. 215:403-10.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

“Binding” as used herein (e.g. with reference to an RNA-binding domain of a polypeptide, binding to a target nucleic acid, and the like) refers to a non-covalent interaction between macromolecules (e.g., between a protein and a nucleic acid; between a CAS polypeptide/guide RNA complex and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (KD) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower KD.

By “binding domain” it is meant a protein or nucleic acid domain that is able to bind non-covalently to another molecule. A binding domain can bind to, for example, a DNA molecule (a DNA-binding domain), an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

The term, “catalytically inactive effector protein,” as used herein, refers to an effector protein that is modified relative to a naturally-occurring effector protein to have a reduced or eliminated catalytic activity relative to that of the naturally-occurring effector protein, but retains its ability to interact with a guide nucleic acid. The catalytic activity that is reduced or eliminated is often a nuclease activity but can be nickase activity. The naturally-occurring effector protein may be a wild-type protein. In some instances, the catalytically inactive effector protein is referred to as a catalytically inactive variant of an effector protein, e.g., a Cas effector protein.

The term, “cis cleavage,” as used herein, refers to cleavage (hydrolysis of a phosphodiester bond) of a target nucleic acid by a complex of an effector protein and a guide nucleic acid, wherein at least a portion of the guide nucleic acid is hybridized to at least a portion of the target nucleic acid. Cleavage may occur within or directly adjacent to the portion of the target nucleic acid that is hybridized to the portion of the guide nucleic acid.

The terms, “cleave,” “cleaving,” and “cleavage,” as used herein, with reference to a nucleic acid molecule or nuclease activity of an effector protein, refer to the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein.

The terms, “complementary” and “complementarity,” as used herein, with reference to a nucleic acid molecule or nucleotide sequence, refer to the characteristic of a polynucleotide having nucleotides that base pair with their Watson-Crick counterparts (C with G; or A with T) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the ‘reverse complement’ sequence or the ‘reverse complementary’ sequence is understood as the sequence of the lower strand in the direction of its 5′- to its 3′-end. Each nucleotide in a double stranded DNA or RNA molecule that is paired with its Watson-Crick counterpart called its complementary nucleotide.

The term, “conservative substitution” as used herein refers to the replacement of one amino acid for another such that the replacement takes place within a family of amino acids that are related in their side chains. Conversely, the term “non-conservative substitution” as used herein refers to the replacement of one amino acid residue for another that does not have a related side chain. Genetically encoded amino acids can be divided into four families having related side chains: (1) acidic (negatively charged): Asp (D), Glu (G); (2) basic (positively charged): Lys (K), Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser(S), Thr (T). Amino acids may be related by aliphatic side chains: Gly (G), Ala (A), Val (V), Leu (L), Ile (I), Ser(S), Thr (T), with Ser(S) and Thr (T) optionally being grouped separately as aliphatic-hydroxyl. Amino acids may be related by aromatic side chains: Phe (F), Tyr (Y), Trp (W). Amino acids may be related by amide side chains: Asn (N), Glu (Q). Amino acids may be related by sulfur-containing side chains: Cys (C) and Met (M).

The term, “cleavage assay,” as used herein, refers to an assay designed to visualize, quantitate or identify cleavage of a nucleic acid. In some instances, the cleavage activity may be cis-cleavage activity.

The term, “clustered regularly interspaced short palindromic repeats (CRISPR),” as used herein, refers to a segment of DNA found in the genomes of certain prokaryotic organisms, including some bacteria and archaea, that includes repeated short sequences of nucleotides interspersed at regular intervals between unique sequences of nucleotides derived from another organism.

The term, “donor nucleic acid,” as used herein, refers to a nucleic acid that is (designed or intended to be) incorporated into a target nucleic acid or target sequence.

The term, “% editing efficiency,” as used herein, refers to the percent of target nucleic acids in a sample or population of cells exhibiting an edited target nucleic acid. Editing efficiency may also be referred to as % editing level or % edited. There are multiple approaches to evaluate % editing efficiency, including, but not limited to, next generation sequence and real time PCR.

The term, “target nucleic acid,” as used herein, refers to a nucleic acid that is selected as the nucleic acid for modification, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The term, “target sequence,” as used herein, when used in reference to a target nucleic acid, refers to a sequence of nucleotides found within a target nucleic acid. Such a sequence of nucleotides can, for example, hybridize to a respective length portion of a guide nucleic acid. Hybridization of the guide nucleic acid to the target sequence may bring an effector protein into contact with the target nucleic acid.

A nucleotide sequence that “encodes” a particular polypeptide or protein, is a nucleotide sequence that is transcribed into mRNA (in the case of DNA) and/or is translated (in the case of mRNA) into a polypeptide.

The term “transgene” as used herein refers to a nucleotide sequence that is inserted into a cell for expression of said nucleotide sequence in the cell. A transgene is meant to include (1) a nucleotide sequence that is not naturally found in the cell (e.g., a heterologous nucleotide sequence); (2) a nucleotide sequence that is a mutant form of a nucleotide sequence naturally found in the cell into which it has been introduced; (3) a nucleotide sequence that serves to add additional copies of the same (e.g., exogenous or homologous) or a similar nucleotide sequence naturally occurring in the cell into which it has been introduced; or (4) a silent naturally occurring or homologous nucleotide sequence whose expression is induced in the cell into which it has been introduced. A donor nucleic acid can comprise a transgene. The cell in which transgene expression occurs can be a target cell, such as a host cell.

The term, “functional fragment,” as used herein, refers to a fragment of a protein that retains some function relative to the entire protein. Non-limiting examples of functions are nucleic acid binding, protein binding, nuclease activity, nickase activity, deaminase activity, demethylase activity, or acetylation activity. A functional fragment may be a recognized functional domain, e.g., a catalytic domain such as, but not limited to, a RuvC domain.

The terms, “fusion effector protein,” “fusion protein,” and “fusion polypeptide,” may be used interchangeably herein and refer to a protein comprising at least two heterologous polypeptides. Often a fusion effector protein comprises an effector protein and a fusion partner protein. In general, the fusion partner protein is not an effector protein. Examples of fusion partner proteins are provided herein.

The terms “fusion partner protein” or “fusion partner,” as used herein, refer to a protein, polypeptide or peptide that is fused, or linked via a linker, to an effector protein. The fusion partner generally imparts some function to the fusion protein that is not provided by the effector protein.

The term, “effector protein,” as used herein, refers to a polypeptide that non-covalently binds to a guide nucleic acid to form a complex that contacts a target nucleic acid, wherein at least a portion of the guide nucleic acid hybridizes to a target sequence of the target nucleic acid. A complex between an effector protein and a guide nucleic acid can include multiple effector proteins or a single effector protein. In some instances, the effector protein modifies the target nucleic acid when the complex contacts the target nucleic acid. In some instances, the effector protein does not modify the target nucleic acid, but it is fused to a fusion partner protein that modifies the target nucleic acid when the complex contacts the target nucleic acid. A non-limiting example of an effector protein modifying a target nucleic acid is cleaving of a phosphodiester bond of the target nucleic acid. Additional examples of modifications an effector protein can make to target nucleic acids are described herein and throughout.

The term, “genetic disease,” as used herein, refers to a disease, disorder, condition, or syndrome associated with or caused by one or more mutations in the DNA of an organism having the genetic disease.

The term, “guide nucleic acid,” as used herein, refers to at least one nucleic acid comprising: a first nucleotide sequence that complexes to an effector protein on either the 5′ or 3′ terminus and the first nucleotide sequence can be fused to a second nucleotide sequence that hybridizes to a target nucleic acid. The first sequence may be referred to herein as a repeat sequence or guide sequence. The second sequence may be referred to herein as a spacer sequence. A guide nucleic acid may be referred to interchangeably with the term, “guide RNA.” It is understood that guide nucleic acids may comprise DNA, RNA, or a combination thereof (e.g., RNA with a thymine base). Guide nucleic acids may include a chemically modified nucleobase or phosphate backbone.

The term, “extended guide RNA (rtgRNA),” as used herein refers to a single nucleic acid molecule comprising (not necessarily in the following order) (1) a guide RNA comprising (a) a protein binding sequence and (b) a spacer sequence; (2) optionally, a linker; and (3) a template RNA (rttRNA) comprising (a) a primer binding sequence and (b) a template sequence. In some embodiments, the orientation of the rtgRNA from 5′ to 3′ is: guide nucleic acid, optional linker, and template RNA. In some embodiments, the orientation of the rtgRNA from 5′ to 3′ is: template RNA, linker, and guide RNA. It is understood that extended guide RNAs may comprise DNA, RNA, or a combination thereof (e.g., RNA with a thymine base).

The term “template RNA (rttRNA)” as used herein, refers to a nucleic acid comprising: a primer binding sequence and a template sequence. It is understood that template RNAs may comprise DNA, RNA, or a combination thereof (e.g., RNA with a thymine base). In some instances, the template RNA is linked to a guide RNA via a linker sequence to form an rtgRNA. Template RNA is used interchangeable with rttRNA and retRNA herein.

The term, “template sequence,” as used herein, refers to a portion of a rttRNA that contains a desired nucleotide modification relative to a target sequence or portion thereof. By way of non-limiting example, the desired edit may comprise one or more nucleotide insertions, deletions or substitutions relative to a target sequence or portion thereof. In some embodiments, it is identical to, complementary to, or reverse complementary to a target sequence or portion thereof. In some embodiments, the template sequence is complementary to a sequence of the target nucleic acid that is adjacent to a nick site of a target site to be edited, with the exception that it includes a desired edit. The template sequence (also referred in some instances as the RT template (RTT)) can be complementary to at least a portion of the target sequence with the exception of at least one nucleotide.

The terms, “primer binding sequence (PBS),” as used herein, refer to a portion of a rttRNA and serves to bind to a primer sequence of the target nucleic acid. In some embodiments, the primer binding sequence binds to a primer sequence in the target nucleic acid that is formed after the target nucleic acid is cleaved by an effector protein. In some embodiments, the primer binding sequence is linked to the 3′ end of an rttRNA. In some embodiments, the primer binding sequence is located at the 5′ end of a rttRNA.

“Primer sequence” as used herein refers to a portion of the target nucleic acid that is capable of hybridizing with the primer binding sequence portion of an rttRNA that is generated after cleavage of the target nucleic acid by an effector protein described herein.

The term “handle sequence,” as used herein, refers to a sequence that binds non-covalently with an effector protein. A handle sequence may also be referred to herein as a “scaffold sequence”. In some instances, the handle sequence comprises all, or a portion of, a repeat sequence. In general, a single guide nucleic acid, also referred to as a single guide RNA (sgRNA), comprises a handle sequence that is capable of being non-covalently bound by an effector protein. The nucleotide sequence of a handle sequence may contain a portion of a tracrRNA, but generally does not comprise a sequence that hybridizes to a repeat sequence, also referred to as a repeat hybridization sequence.

The term “trans-activating RNA (tracrRNA),” as used herein, refers to a nucleic acid that comprises a first sequence that is capable of being non-covalently bound by an effector protein, and a second sequence that hybridizes to a portion of a crRNA, which may be referred to as a repeat hybridization sequence.

The terms, “CRISPR RNA” or “crRNA,” as used herein, refer to a type of guide nucleic acid, wherein the nucleic acid is RNA comprising a first sequence, often referred to herein as a spacer sequence, that hybridizes to a target sequence of a target nucleic acid, and a second sequence, often referred to herein as a repeat sequence or guide sequence, that interacts with an effector protein. In some instances, the second sequence is bound by the effector protein. In some instances, the second sequence hybridizes to a portion of a tracrRNA, wherein the tracrRNA forms a complex with the effector protein.

The term, “extension,” as used herein refers to additional nucleotides added to a nucleic acid, RNA, or DNA, or additional amino acids added to a peptide, polypeptide, or protein. Extensions may be processed during the formation of the guide RNA. In some instances, the extension comprises or consists of a template RNA.

By “hybridizable” or “complementary” or “substantially complementary” it is meant that a nucleic acid (e.g. RNA, DNA) comprises a sequence of nucleotides that enables it to noncovalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) [DNA, RNA]. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, in the context of this disclosure, a guanine (G) (e.g., of dsRNA duplex of a guide RNA molecule; of a guide RNA base pairing with a target nucleic acid, etc.) is considered complementary to both a uracil (U) and to an adenine (A). For example, when a G/U base-pair can be made at a given nucleotide position of a dsRNA duplex of a guide RNA molecule, the position is not considered to be non-complementary, but is instead considered to be complementary.

The conditions of temperature and ionic strength determine the “stringency” of the hybridization. Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. For hybridizations between nucleic acids with short stretches of complementarity (e.g., complementarity over 35 or less, 30 or less, 25 or less, 22 or less, 20 or less, or 18 or less nucleotides) the position of mismatches can become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Temperature, wash solution salt concentration, and other conditions may be adjusted as necessary according to factors such as length of the region of complementation and the degree of complementation.

While hybridization typically occurs between two nucleotide sequences that are complementary, mismatches between bases are possible. It is understood that two nucleotide sequences need not be 100% complementary to be specifically hybridizable, or for hybridization to occur. Moreover, a nucleotide sequence may hybridize over one or more segments such that intervening or adjacent segments are not involved in the hybridization event (e.g., a bulge, a loop structure or hairpin structure, etc.). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656), the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489), and the like.

The conditions appropriate for hybridization between two nucleotide sequences depend on the length of the sequence and the degree of complementarity, variables well known in the art. The greater the degree of complementarity between two nucleotide sequences, the greater the value of the melting temperature (Tm) for hybrids of nucleic acids having those sequences. For hybridizations between nucleic acids with short stretches of complementarity (e.g., complementarity over 35 or less, 30 or less, 25 or less, 22 or less, 20 or less, or 18 or less nucleotides) the position of mismatches can become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Temperature, wash solution salt concentration, and other conditions may be adjusted as necessary according to factors such as length of the region of complementation and the degree of complementation. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

The term, “heterologous,” as used herein, with reference to at least two different polypeptide sequences, means that the two different polypeptide sequences are not found similarly connected to one another in a native nucleic acid or protein. A protein that is heterologous to the effector protein is a protein that is not covalently linked via an amide bond to the effector protein in nature. In some instances, a heterologous protein is not encoded by a species that encodes the effector protein. A guide nucleic acid may comprise a first sequence and a second sequence, wherein the first sequence and the second sequence are not found covalently linked via a phosphodiester bond in nature. Thus, the first sequence is considered to be heterologous with the second sequence, and the guide nucleic acid may be referred to as a heterologous guide nucleic acid.

The term “linked” as used herein in reference to an amino acid or nucleic acid sequence refers to any covalent mechanism by which two amino acid sequences or nucleic acid sequences are connected to each other in sequence. For example, in some embodiments, two sequences are linked directly together by a covalent bond (e.g., an amide bond or phosphodiester bond). In some embodiments, two sequences are linked together by a peptide or nucleic acid linker.

The term, “linked amino acids” as used herein, refers to at least two amino acids linked by an amide bond or a peptide bond.

The term, “linker,” as used herein, refers to an amino acid sequence or nucleic acid sequence that links a first polypeptide to a second polypeptide or a first nucleic acid to a second nucleic acid.

The term, “modified target nucleic acid,” as used herein, refers to a target nucleic acid, wherein the target nucleic acid has undergone a modification, for example, after contact with an effector protein. In some instances, the modification is an alteration in the sequence of the target nucleic acid. In some instances, the modified target nucleic acid comprises an insertion, deletion, or replacement of one or more nucleotides compared to the unmodified target nucleic acid.

The terms “peptide,” “polypeptide,” and “protein” are used interchangeably herein, refer to a polymeric form of amino acids. A polypeptide may include coded and non-coded amino acids, chemically or biochemically modified or derivatized amino acids, and polypeptides having modified peptide backbones. Accordingly, polypeptides as described herein may comprise one or more mutations, one or more sequence modifications, or both. A peptide generally has a length of 100 or fewer linked amino acids.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways.

A DNA sequence that “encodes” a particular RNA is a DNA nucleotide sequence that is transcribed into RNA. A DNA polynucleotide may encode an RNA (mRNA) that is translated into protein (and therefore the DNA and the mRNA both encode the protein), or a DNA polynucleotide may encode an RNA that is not translated into protein (e.g. tRNA, rRNA, microRNA (miRNA), a “non-coding” RNA (ncRNA), a guide RNA, etc.).

A “protein coding sequence” or a sequence that encodes a particular protein or polypeptide, is a nucleotide sequence that is transcribed into mRNA (in the case of DNA) and is translated (in the case of mRNA) into a polypeptide in vitro or in vivo when placed under the control of appropriate regulatory sequences.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence (e.g., RNA-guided endonuclease and the like) and/or regulate translation of an encoded polypeptide.

The term, “promoter” or “promoter sequence,” is a DNA regulatory region capable of binding RNA polymerase and initiating transcription of a downstream (3′ direction) coding or non-coding sequence. A transcription initiation site, as well as protein binding domains responsible for the binding of RNA polymerase, can also be found in a promoter region. Eukaryotic promoters will often, but not always, contain “TATA” boxes and “CAT” boxes. Various promoters, including inducible promoters, may be used to drive expression by the various vectors of the present disclosure.

The term, “protospacer adjacent motif (PAM),” as used herein, refers to a nucleotide sequence found in a target nucleic acid that directs an effector protein to modify the target nucleic acid at a specific location. In some instances, a PAM is required for a complex of an effector protein and a guide nucleic acid to hybridize to and modify the target nucleic acid. In some instances, the complex does not require a PAM to modify the target nucleic acid. One example of a PAM sequences is NTTN, where N can be any nucleic acid.

The term “RNA-dependent DNA polymerase (RDDP),” as used herein, refers to a DNA polymerase that uses a single-stranded RNA as a template for the synthesis of a complementary DNA strand.

The term, “RuvC” domain as used herein refers to a region of an effector protein that is capable of cleaving a target nucleic acid, and in certain instances, of processing a pre-crRNA. In some instances, the RuvC domain is located near the C-terminus of the effector protein. A single RuvC domain may comprise RuvC subdomains, for example a RuvCI subdomain, a RuvCII subdomain and a RuvCIII subdomain. The term “RuvC” domain can also refer to a “RuvC-like” domain. Various RuvC-like domains are known in the art and are easily identified using online tools such as InterPro (https://www.ebi.ac.uk/interpro/). For example, a RuvC-like domain may be a domain which shares homology with a region of TnpB proteins of the IS605 and other related families of transposons.

The term, “nickase” as used herein refers to an enzyme that possess catalytic activity for single stranded nucleic acid cleavage of a double stranded nucleic acid. A nickase cleaves a phosphodiester bond between two nucleotides of only one strand of dsDNA.

The terms, “nuclease” and “endonuclease” are used interchangeably herein to mean an enzyme which possesses catalytic activity for nucleic acid cleavage.

The term, “nuclease activity,” is used to refer to catalytic activity that results in nucleic acid cleavage (e.g., ribonuclease activity (ribonucleic acid cleavage), or deoxyribonuclease activity (deoxyribonucleic acid cleavage), etc.).

The term “naturally-occurring,” “unmodified,” or “wild type” as used herein as applied to a nucleic acid, a polypeptide, a cell, or an organism, refers to a nucleic acid, polypeptide, cell, or organism that is found in nature. For example, a polypeptide or polynucleotide sequence that is present in an organism that can be isolated from a source in nature is naturally occurring.

The terms, “non-naturally occurring” and “engineered,” as used herein, are used interchangeably and indicate the involvement of the hand of man. The terms, when referring to a nucleic acid, nucleotide, protein, polypeptide, peptide or amino acid, refer to a molecule, such as but not limited to, a nucleic acid, nucleotide, protein, polypeptide, peptide or amino acid refers to a modification of that molecule (e.g., chemical modification, nucleotide sequence, or amino acid sequence) that is not present in the naturally molecule. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by the hand of man.

The term, “variant,” is intended to mean a form or version of a protein that differs from the wild-type protein. A variant may have a different function or activity relative to the wild-type protein.

The term, “sequence modification,” as used herein refers to a modification of one or more nucleic acid residues of a nucleotide sequence or one or more amino acid residue of an amino acid sequence, such as chemical modification of one or more nucleobases; or chemical modifications to the phosphate backbone, a nucleotide, a nucleobase, or a nucleoside. Such modifications can be made to an effector protein amino acid sequence or guide nucleic acid nucleotide sequence, or any sequence disclosed herein (e.g., a nucleic acid encoding an effector protein or a nucleic acid that, when transcribed, produces a guide nucleic acid). Methods of modifying a nucleic acid or amino acid sequence are known. One of ordinary skill in the art will appreciate that the sequence modification(s) may be located at any position(s) of a nucleic acid such that the function of the nucleic acid, protein, composition or system is not substantially decreased. Nucleic acids provided herein can be prepared according to any available technique including, but not limited to chemical synthesis, enzymatic synthesis, which is generally termed in vitro-transcription, cloning, enzymatic, or chemical cleavage, etc. In some instances, the nucleic acids provided herein are not uniformly modified along the entire length of the molecule. Different nucleotide modifications and/or backbone structures can exist at various positions within the nucleic acid.

The term, “nucleic acid expression vector,” as used herein, refers to a plasmid that can be used to express a nucleic acid of interest.

The term, “nuclear localization signal (NLS),” as used herein, refers to an entity (e.g., peptide) that facilitates localization of a nucleic acid, protein, or small molecule to the nucleus, when present in a cell that contains a nuclear compartment.

A person of ordinary skill in the art would appreciate that referring to a “nucleotide(s)”, and/or “nucleoside(s)”, in the context of a nucleic acid molecule having multiple residues, is interchangeable and describe the sugar and base of the residue contained in the nucleic acid molecule. Similarly, a skilled artisan could understand that linked nucleotides and/or linked nucleosides, as used in the context of a nucleic acid having multiple linked residues, are interchangeable and describe linked sugars and bases of residues contained in a nucleic acid molecule. When referring to a “nucleobase(s)”, or linked nucleobase, as used in the context of a nucleic acid molecule, it can be understood as describing the base of the residue contained in the nucleic acid molecule, for example, the base of a nucleotide, nucleosides, or linked nucleotides or linked nucleosides. A person of ordinary skill in the art when referring to nucleotides, nucleosides, and/or nucleobases would also understand the differences between RNA and DNA (generally the exchange of uridine for thymidine or vice versa) and the presence of nucleoside analogs, such as modified uridines, do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, NI-methyl pseudouridine, or 5-methoxyuridine, is considered 100% identical to AUG in that both are perfectly complementary to the same sequence (5′-CAU).

Thus, the term “recombinant” polypeptide does not necessarily refer to a polypeptide whose amino acid sequence does not naturally occur. Instead, a “recombinant” polypeptide is encoded by a recombinant non-naturally occurring DNA sequence, but the amino acid sequence of the polypeptide can be naturally occurring (“wild type”) or non-naturally occurring (e.g., a variant, a mutant, etc.). A recombinant polypeptide is the product of a process run by a human or machine.

A “vector” or “expression vector” is a replicon, such as plasmid, phage, virus, artificial chromosome, or cosmid, to which another DNA segment, i.e., an “insert”, may be attached so as to bring about the replication of the attached segment in a cell.

The term “viral vector,” as used herein, refers to a nucleic acid to be delivered into a host cell via a recombinantly produced virus or viral particle.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence (or the coding sequence can also be said to be operably linked to the promoter) if the promoter affects its transcription or expression.

The terms “recombinant expression vector,” or “DNA construct” are used interchangeably herein to refer to a DNA molecule comprising a vector and an insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

A cell has been “genetically modified,” “transformed,” or “transfected” by exogenous DNA or exogenous RNA, e.g., a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13. pii: S0169-409X (12) 00283-9. doi: 10.1016/j.addr.2012.09.023), and the like.

The choice of method of genetic modification is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995.

“Nuclease” and “endonuclease” are used interchangeably herein to mean an enzyme which possesses catalytic activity for nucleic acid cleavage (e.g., ribonuclease activity (ribonucleic acid cleavage), deoxyribonuclease activity (deoxyribonucleic acid cleavage), etc.).

By “cleavage domain,” “active domain,” or “nuclease domain” of a nuclease it is meant the polypeptide sequence or domain within the nuclease which possesses the catalytic activity for nucleic acid cleavage. A cleavage domain can be contained in a single polypeptide chain or cleavage activity can result from the association of two (or more) polypeptides. A single nuclease domain may consist of more than one isolated stretch of amino acids within a given polypeptide.

The term “stem cell” is used herein to refer to a cell (e.g., plant stem cell, vertebrate stem cell) that has the ability both to self-renew and to generate a differentiated cell type (see Morrison et al. (1997) Cell 88:287-298). In the context of cell ontogeny, the adjective “differentiated”, or “differentiating” is a relative term. A “differentiated cell” is a cell that has progressed further down the developmental pathway than the cell it is being compared with. Thus, pluripotent stem cells (described below) can differentiate into lineage-restricted progenitor cells (e.g., mesodermal stem cells), which in turn can differentiate into cells that are further restricted (e.g., neuron progenitors), which can differentiate into end-stage cells (i.e., terminally differentiated cells, e.g., neurons, cardiomyocytes, etc.), which play a characteristic role in a certain tissue type and may or may not retain the capacity to proliferate further. Stem cells may be characterized by both the presence of specific markers (e.g., proteins, RNAs, etc.) and the absence of specific markers. Stem cells may also be identified by functional assays both in vitro and in vivo, particularly assays relating to the ability of stem cells to give rise to multiple differentiated progeny.

Stem cells of interest include pluripotent stem cells (PSCs). The term “pluripotent stem cell” or “PSC” is used herein to mean a stem cell capable of producing all cell types of the organism. Therefore, a PSC can give rise to cells of all germ layers of the organism (e.g., the endoderm, mesoderm, and ectoderm of a vertebrate). Pluripotent cells are capable of forming teratomas and of contributing to ectoderm, mesoderm, or endoderm tissues in a living organism. Pluripotent stem cells of plants are capable of giving rise to all cell types of the plant (e.g., cells of the root, stem, leaves, etc.).

PSCs of animals can be derived in a number of different ways. For example, embryonic stem cells (ESCs) are derived from the inner cell mass of an embryo (Thomson et. al, Science. 1998 Nov. 6; 282 (5391): 1145-7) whereas induced pluripotent stem cells (iPSCs) are derived from somatic cells (Takahashi et. al, Cell. 2007 Nov. 30; 131 (5): 861-72; Takahashi et. al, Nat Protoc. 2007; 2 (12): 3081-9; Yu et. al, Science. 2007 Dec. 21; 318 (5858): 1917-20. Epub 2007 Nov. 20). Because the term PSC refers to pluripotent stem cells regardless of their derivation, the term PSC encompasses the terms ESC and iPSC, as well as the term embryonic germ stem cells (EGSC), which are another example of a PSC. PSCs may be in the form of an established cell line, they may be obtained directly from primary embryonic tissue, or they may be derived from a somatic cell. PSCs can be target cells of the methods described herein.

By “induced pluripotent stem cell” or “iPSC” it is meant a PSC that is derived from a cell that is not a PSC (i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERI, and zfp42. Examples of methods of generating and characterizing iPSCs may be found in, for example, U.S. Patent Publication Nos. US20090047263, US20090068742, US20090191159, US20090227032, US20090246875, and US20090304646, the disclosures of which are incorporated herein by reference. Generally, to generate iPSCs, somatic cells are provided with reprogramming factors (e.g., Oct4, SOX2, KLF4, MYC, Nanog, Lin28, etc.) known in the art to reprogram the somatic cells to become pluripotent stem cells.

By “somatic cell” it is meant any cell in an organism that, in the absence of experimental manipulation, does not ordinarily give rise to all types of cells in an organism. In other words, somatic cells are cells that have differentiated sufficiently that they will not naturally generate cells of all three germ layers of the body, i.e., ectoderm, mesoderm and endoderm. For example, somatic cells would include both neurons and neural progenitors, the latter of which may be able to naturally give rise to all or some cell types of the central nervous system but cannot give rise to cells of the mesoderm or endoderm lineages.

The terms, “treatment” or “treating,” as used herein, are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

A “syndrome”, as used herein, refers to a group of symptoms which, taken together, characterize a condition.

The terms “individual,” “subject,” “host,” and “patient,” used interchangeably herein, refer to an individual organism, e.g., a mammal, including, but not limited to, murines, simians, humans, non-human primates, ungulates, felines, canines, bovines, ovines, mammalian farm animals, mammalian sport animals, and mammalian pets.

The term, “subject,” as used herein, refers to an animal. The subject may be a mammal. The subject may be a human. The subject may be diagnosed or at risk for a disease.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

### Introduction

In some embodiments, the present disclosure provides systems and methods comprising: a) at least one of a polypeptide and/or a nucleic acid encoding the polypeptide; and b) at least one of a guide nucleic acid and/or a DNA molecule encoding the guide nucleic acid, and uses thereof, wherein the polypeptide and the guide nucleic acid form a complex that binds a target nucleic acid.

Polypeptides described herein may bind and, optionally, cleave nucleic acids in a sequence-specific manner. Polypeptides described herein may bind a target region of a target nucleic acid and cleave the target nucleic acid within the target region or at a position adjacent to the target region. In some embodiments, a polypeptide is activated when it binds a target region of a target nucleic acid to cleave a region of the target nucleic acid that is near, but not adjacent to the target region. A polypeptide may be an effector protein, such as a CRISPR-associated (Cas) protein, which may be coupled to a guide nucleic acid that imparts activity or sequence selectivity to the polypeptide. In general, guide nucleic acids comprise a first sequence that is at least partially complementary to a target nucleic acid, which may be referred to as a spacer sequence. In some embodiments, compositions, systems, and methods comprising effector proteins and guide nucleic acids can further comprise a second sequence, at least a portion of which interacts with the polypeptide. In some instances, the second sequence comprises a sequence that is similar or identical to a portion of a tracrRNA sequence, a CRISPR repeat sequence, or a combination thereof. In some embodiments, the guide nucleic acid does not comprise a tracrRNA.

Effector proteins disclosed herein may cleave nucleic acids, including single stranded RNA (ssRNA), double stranded DNA (dsDNA), and single-stranded DNA (ssDNA). Polypeptides disclosed herein may provide cis cleavage activity, trans cleavage activity, nickase activity, or a combination thereof. Cis cleavage activity is cleavage of a target nucleic acid that is hybridized to a guide nucleic acid (crRNA or sgRNA), wherein cleavage occurs within or directly adjacent to the region of the target nucleic acid that is hybridized to the guide nucleic acid. Nickase activity is the selective cleavage of one strand of a dsDNA molecule.

The compositions, systems and methods described herein are non-naturally occurring. In some instances, compositions, systems and methods comprise a guide nucleic acid or a use thereof. In some instances, compositions, systems and methods comprise an engineered polypeptide or a use thereof. In general, compositions and systems described herein are not found in nature. In some embodiments, compositions, methods and systems described herein comprise at least one non-naturally occurring component. For example, disclosed compositions, methods and systems may comprise a guide nucleic acid, wherein the sequence of the guide nucleic acid is different or modified from that of a naturally-occurring guide nucleic acid. In some embodiments, compositions, systems and methods comprise at least two components that do not naturally occur together. For example, disclosed compositions, methods and systems may comprise a guide nucleic acid comprising a repeat region and a spacer region which do not naturally occur together. Also, by way of non-limiting example, disclosed compositions, methods and systems may comprise a guide nucleic acid and an effector protein that do not naturally occur together. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

In some embodiments, the guide nucleic acid comprises a non-natural nucleotide sequence. In some embodiments, the non-natural nucleotide sequence is a nucleotide sequence that is not found in nature. The non-natural nucleotide sequence may comprise a portion of a naturally-occurring sequence, wherein the portion of the naturally-occurring sequence is not present in nature absent the remainder of the naturally-occurring sequence. In some embodiments, the guide nucleic acid comprises two naturally-occurring sequences arranged in an order or proximity that is not observed in nature. In some embodiments, compositions and systems comprise a ribonucleotide complex comprising an effector protein and a guide nucleic acid that do not occur together in nature. Engineered guide nucleic acids may comprise a first sequence and a second sequence that do not occur naturally together. For example, a guide nucleic acid may comprise a sequence of a naturally-occurring repeat region and a spacer region that is complementary to a naturally-occurring eukaryotic sequence. The guide nucleic acid may comprise a sequence of a repeat region that occurs naturally in an organism and a spacer region that does not occur naturally in that organism. A guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. In some embodiments, the guide nucleic acid comprises two heterologous sequences arranged in an order or proximity that is not observed in nature. Therefore, compositions and systems described herein are not naturally occurring.

### Precision Editing Systems

In some embodiments, the present disclosure provides compositions, systems, and methods for precision editing. The present disclosure provides each of the components for such precision editing systems (e.g., effector proteins, RDDPs, and rtgRNAs) as well as systems comprising the individual components. In some embodiments, the effector protein and the RDDP are provided in the form of a fusion protein. In some embodiments, the effector protein and the RDDP are provided as two separate proteins. In some embodiments, the effector protein (or fusion protein comprising an effector protein) forms a complex with an extended guide RNA (rtgRNA).

In some instances, the effector protein is fused to an RDDP. In some instances, the RDDP comprises a reverse transcriptase. The effector protein may nick a strand of the target nucleic acid and the RDDP synthesizes new DNA off the nicked end, wherein the new DNA is complementary to the template sequence (RT template), thereby producing the desired genomic edit in the target nucleic acid. In some instances, the effector protein nicks the target strand. In some instances, the effector protein nicks the non-target strand.

In some embodiments, the effector protein nicks both the target strand and the non-target strand sequentially. For example, in some embodiments, the effector protein nicks the target strand first and facilitates, with an rtgRNA, the introduction of the desired genomic edit. After editing, the effector protein, in combination with a guide RNA that targets the non-target strand, can nick the non-target strand. The cellular DNA repair mechanisms can then repair the non-target strand using the edited target strand as template. See Anzalone, Nature. 2019 December; 576 (7785): 149-157.

In some embodiments, the systems provided herein comprise (a) a fusion protein described herein (or a polynucleotide encoding the same); and (b) an rtgRNA comprising a guide RNA and a template RNA. In some embodiments, the systems provided herein comprise an RDDP comprising (a) an amino acid sequence that is at least 90% or at least 95% identical to any one of SEQ ID NOs: 485-530; (b) an effector protein; and (c) an rtgRNA comprising a guide RNA and a template RNA.

In some embodiments, the systems provided herein comprise (a) a fusion protein described herein (or a polynucleotide encoding the same); (b) an rtgRNA comprising a guide RNA and a template RNA; and (c) a guide RNA. In some embodiments, the systems provided herein comprise an RDDP comprising (a) an amino acid sequence that is at least 90% or at least 95% identical to any one of SEQ ID NOs: 485-530; (b) an effector protein; (c) an rtgRNA comprising a guide RNA and a template RNA; and (d) a guide RNA.

In some embodiments, compositions, systems, and methods comprise a fusion protein or uses thereof. In general, a fusion protein comprises an effector protein that is covalently linked to a partner protein that is heterologous to the effector protein. In some embodiments, a partner protein comprises an enzyme that is capable of catalyzing the modification (insertion, deletion, or base-to-base conversion) of a target nucleic acid. In some instances, the partner protein is a polymerase. In some instances, the partner protein is an RNA-directed DNA polymerase (RDDP). In some instances, the RDDP is a reverse transcriptase. In some instances, compositions, systems, and methods disclosed herein, comprise an effector protein and a partner protein, or uses thereof, wherein the effector protein and the partner protein are not covalently linked.

In some instances, the enzyme that is capable of catalyzing the modification of the target nucleic acid forms a complex with an extended guide RNA (rtgRNA). In some embodiments, the extended guide RNA comprises (not necessarily in this order): a first region (also referred to as a protein binding region or protein binding sequence) that interacts with an effector protein; a second region comprising a spacer sequence that is complementary to a target sequence of a first strand of a target dsDNA molecule; a third region comprising a template sequence that is complementary to at least a portion of the target sequence on the non-target strand of the target dsDNA molecule with the exception of at least one nucleotide; and a fourth region comprising a primer binding sequence that hybridizes to a primer sequence of the target dsDNA molecule that is formed when target nucleic acid is cleaved. The third region or template sequence may comprise a nucleotide having a different nucleobase than that of a nucleotide at the corresponding position in the target nucleic acid when the template sequence and the target sequence are aligned for maximum identity. In some instances, there is a linker between any one of the first, second, third and fourth regions. In some instances, the linker comprises a nucleotide. In some instances, the linker comprises multiple nucleotides.

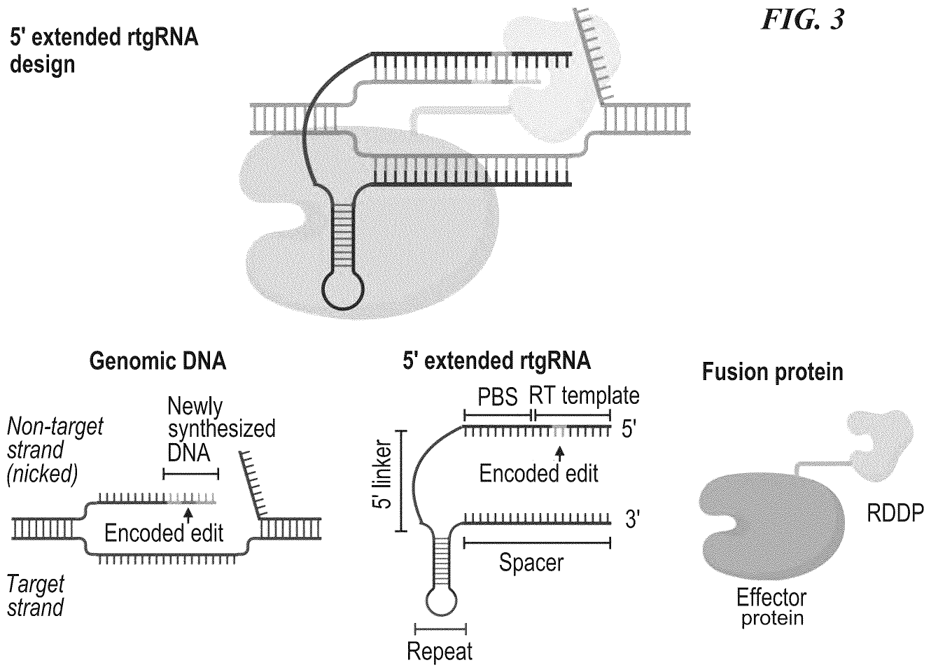

In some instances, the third and fourth regions are 5′ of the first and second regions. In some instances, the order of the regions of the extended guide RNA from 5′ to 3′ is: third region, fourth region, first region, and second region. In some instances, there is a linker between any one of the first, second, third and fourth regions. In some instances, there is a linker between the first and fourth regions. In some instances, the effector protein is fused to an RDDP. In some instances, the RDDP comprises a reverse transcriptase. See, e.g., FIG. 3. The effector protein may nick a strand of the target nucleic acid and the RDDP synthesizes new DNA off the nicked end, wherein the new DNA is complementary to the template sequence (RT template), thereby producing the desired genomic edit in the target nucleic acid. In some instances, the effector protein nicks the target strand. In some instances, the effector protein nicks the non-target strand.

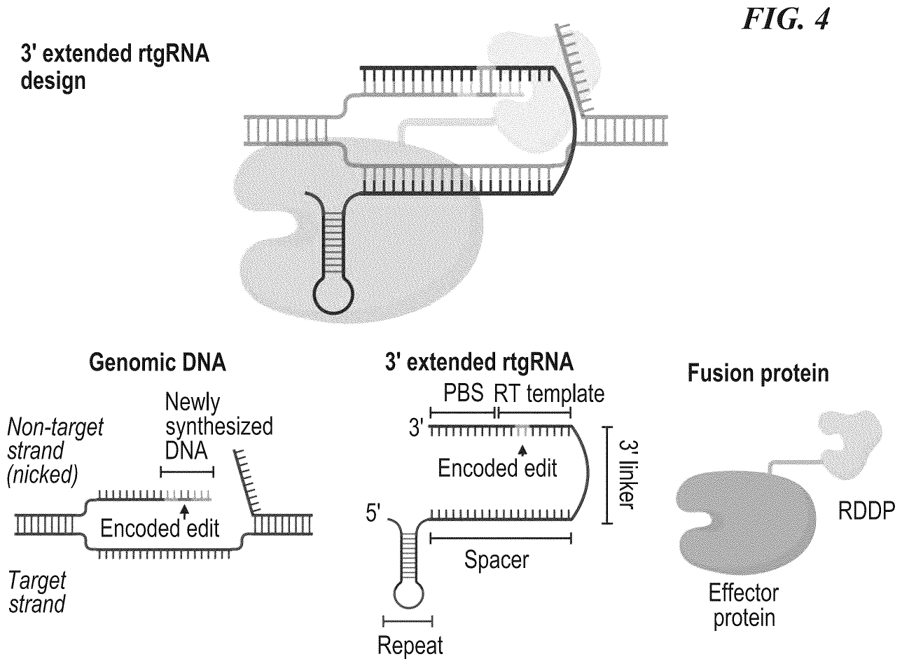

In some instances, the third and fourth regions are 3′ of the first and second regions. In some instances, the order of the regions of the extended guide RNA from 5′ to 3′ is: first region, second region, third region, and fourth region. In some instances, there is a linker between the second and third regions. In some instances, the effector protein is fused to an RDDP. In some instances, the RDDP comprises a reverse transcriptase. See, e.g., FIG. 4. The effector protein may nick a strand of the target nucleic acid and the RDDP synthesizes new DNA off the nicked end, wherein the new DNA is complementary to the template sequence (RT template), thereby producing the desired genomic edit in the target nucleic acid. In some instances, the effector protein nicks the target strand. In some instances, the effector protein nicks the non-target strand.

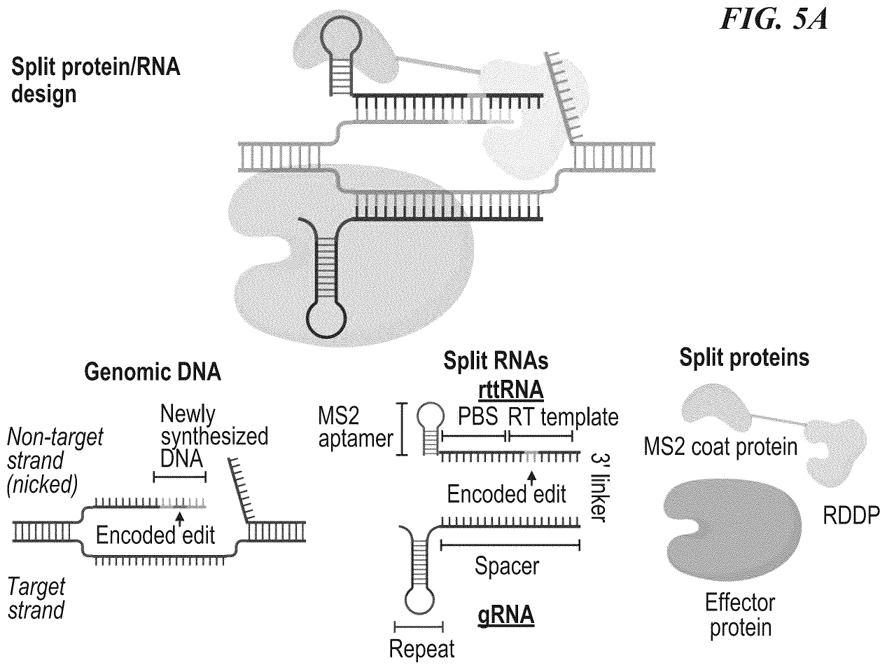

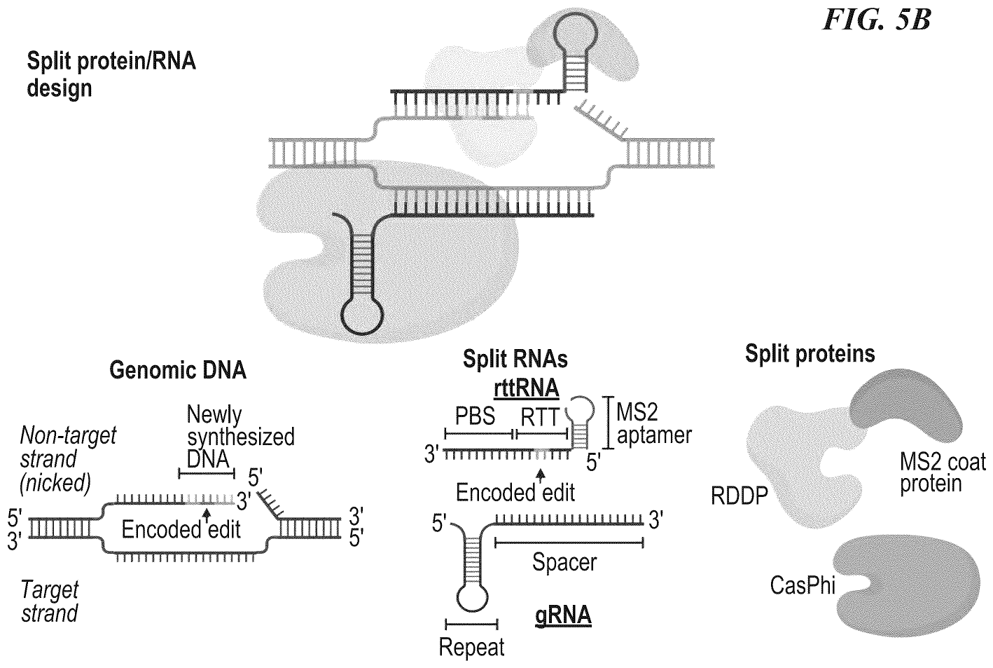

In some instances, compositions and systems comprise (1) a guide RNA comprising (a) a first region (also referred to as a protein binding region or protein binding sequence) that interacts with an effector protein and (b) a second region comprising a spacer sequence that is complementary to a target sequence of a first strand of a target dsDNA molecule; and (2) a template RNA (rttRNA) comprising (a) a primer binding sequence that hybridizes to a primer sequence of the target dsDNA molecule that is formed when the target nucleic acid is cleaved and (b) a template sequence that is complementary to at least a portion of the target sequence on the second strand of the target dsDNA molecule with the exception of at least one nucleotide. The template sequence may comprise a nucleotide having a different nucleobase than that of a nucleotide at the corresponding position in the target nucleic acid when the template sequence and the target sequence are aligned for maximum identity. In some instances, the primer binding sequence is linked to the template sequence. In some instances, the guide RNA and the template RNA are covalently connected. In some instances, the guide RNA and the template RNA are not covalently connected, see, e.g. FIGS. 5A-5B. In some instances, such compositions comprise an effector protein that is fused to a RDDP. In some instances, such compositions comprise an effector protein that is not linked or fused (e.g., covalently linked) to an RDDP. Compositions and systems that comprise an RDDP that is not linked/fused to an effector protein and/or a guide RNA that is not fused/linked to a template RNA may be referred to as a “split protein/RNA design.” Scc, e.g. FIGS. 5A-5B. The effector protein may nick a strand of the target nucleic acid and the RDDP synthesizes new DNA off the nicked end, wherein the new DNA is complementary to the template sequence (RT template), thereby producing the desired genomic edit in the target nucleic acid. In some instances, the effector protein nicks the target strand. In some instances, the effector protein nicks the non-target strand.

In some embodiments, compositions and systems comprise a moiety that that binds an RNA-aptamer. This moiety may be fused to an RDDP and the RNA-aptamer may be linked to the gRNA or template RNA, or a combination thereof. Thus, the moiety may serve to deliver the RDDP to the target sequence and thus, the RDDP need not be linked to the effector protein. By way of non-limiting example, in some embodiments, compositions and systems comprise an MS2 protein localization sequence. In some embodiments, a guide RNA comprises an MS2 protein localization sequence. In some embodiments, the template RNA comprises an MS2 protein localization sequence. The MS2 protein localization sequence may be located at the 5′ or 3′ terminus of the template RNA. In some instances, the RDDP is fused to an MS2 coat protein (or protein that is capable of binding the MS2 protein localization sequence), thereby localizing the RDDP to the MS2 protein localization sequence and therefore, the effector protein, template RNA and/or target nucleic acid. Additional examples of such localizing systems are described by Chen et al., FEBS J. (2013) 280:3734-3754.

### Effector Proteins

Provided herein, are compositions, systems, and methods comprising an effector protein and uses thereof. In general, the effector protein is a CRISPR associated (Cas) protein. In some instances, the effector protein is not a Type II Cas protein. In some instances, the effector protein is not a Cas9 protein. In some instances, the effector protein is a Type V protein. In some embodiments, the Type V effector protein comprises an RuvC domain and does not comprise an HNH domain. In some instances, the effector protein is a Type VU-3 protein. In some instances, the effector protein is a Type VU-4 protein. In some instances, the effector protein is a CasPhi (CasΦ, Cas12J) protein. In some instances, the effector protein is a Cas14 protein. In some instances, the Cas14 protein is a Cas14a protein. In some instances, the Cas14 protein is a Cas14b protein. Additional non-limiting examples of Cas proteins that can be used in the compositions, systems, and methods of the instant disclosure are Cas12 proteins and Cas13 proteins.

In some embodiments, compositions and systems described herein comprise an effector protein that is similar to a naturally occurring effector protein. The effector protein may lack a portion of the naturally occurring effector protein. The effector protein may comprise a mutation relative to the naturally-occurring effector protein, wherein the mutation is not found in nature. The effector protein may also comprise at least one additional amino acid relative to the naturally-occurring effector protein. For example, the effector protein may comprise an addition of a nuclear localization signal relative to the natural occurring effector protein. In certain embodiments, a nucleotide sequence encoding the effector protein is codon optimized (e.g., for expression in a eukaryotic cell) relative to the naturally occurring sequence.

An effector protein may be brought into proximity of a target nucleic acid in the presence of a guide nucleic acid when the guide nucleic acid includes a nucleotide sequence that is complementary with a target sequence in the target nucleic acid. The ability of an effector protein to modify a target nucleic acid may be dependent upon the effector protein being bound to a guide nucleic acid and the guide nucleic acid being hybridized to a target nucleic acid. An effector protein may also recognize a protospacer adjacent motif (PAM) sequence present in the target nucleic acid, which may direct the modification activity of the effector protein. An effector protein may modify a target nucleic acid by cis cleavage or trans cleavage. The modification of the target nucleic acid generated by an effector protein may, as a non-limiting example, result in modulation of the expression of the target nucleic acid (e.g., increasing or decreasing expression of the nucleic acid) or modulation of the activity of a translation product of the target nucleic acid (e.g., inactivation of a protein binding to an RNA molecule or hybridization).

An effector protein may be a CRISPR-associated (“Cas”) protein. An effector protein may function as a single protein, including a single protein that is capable of binding to a guide nucleic acid and modifying a target nucleic acid. Alternatively, an effector protein may function as part of a multiprotein complex, including, for example, a complex having two or more effector proteins, including two or more of the same effector proteins (e.g., dimer or multimer). An effector protein, when functioning in a multiprotein complex, may have only one functional activity (e.g., binding to a guide nucleic acid), while other effector proteins present in the multiprotein complex are capable of the other functional activity (e.g., modifying a target nucleic acid). An effector protein may be a modified effector protein having increased modification activity and/or increased substrate binding activity (e.g., substrate selectivity, specificity, and/or affinity). Alternatively, or in addition, an effector protein may be a catalytically inactive effector protein having reduced modification activity or no modification activity. Accordingly, an effector protein as used herein encompasses a modified polypeptide that does not have nuclease activity.

In certain embodiments, effector proteins described herein comprise one or more functional domains. Effector protein functional domains can include a protospacer adjacent motif (PAM)-interacting domain, an oligonucleotide-interacting domain, one or more recognition domains, a non-target strand interacting domain, and a RuvC domain. A PAM interacting domain can be a target strand PAM interacting domain (TPID) or a non-target strand PAM interacting domain (NTPID). In some embodiments, a PAM interacting domain, such as a TPID or a NTPID, on an effector protein describes a region of an effector protein that interacts with target nucleic acid. In some embodiments, the effector proteins comprise a RuvC domain. In some embodiments, a RuvC domain comprises with substrate binding activity, catalytic activity, or both. In some embodiments, the RuvC domain may be defined by a single, contiguous sequence, or a set of RuvC subdomains that are not contiguous with respect to the primary amino acid sequence of the protein. An effector protein of the present disclosure may include multiple RuvC subdomains, which may combine to generate a RuvC domain with substrate binding or catalytic activity. For example, an effector protein may include three RuvC subdomains (RuvC-I, RuvC-II, and RuvC-III) that are not contiguous with respect to the primary amino acid sequence of the effector protein, but form a RuvC domain once the protein is produced and folds. In some embodiments, effector proteins comprise one or more recognition domain (REC domain) with a binding affinity for a guide nucleic acid or for a guide nucleic acid-target nucleic acid heteroduplex. An effector protein may comprise a zinc finger domain. In some embodiments, the effector protein does not comprise an HNH domain.

An effector protein may be small, which may be beneficial for nucleic acid detection or editing (for example, the effector protein may be less likely to adsorb to a surface or another biological species due to its small size). The smaller nature of these effector proteins may allow for them to be more easily packaged and delivered with higher efficiency in the context of genome editing and more readily incorporated as a reagent in an assay. In some embodiments, the length of the effector protein is at least 300 linked amino acid residues. In some embodiments, the length of the effector protein is at least 325 linked amino acid residues. In some embodiments, the length of the effector protein is at least 350 linked amino acid residues. In some embodiments, the length of the effector protein is at least 375 linked amino acid residues. In some embodiments, the length of the effector protein is at least 400 linked amino acid residues. In some embodiments, the length of the effector protein is at least 425 linked amino acid residues. In some embodiments, the length of the effector protein is at least 450 linked amino acid residues. In some embodiments, the length of the effector protein is not greater than 700 linked amino acid residues. In some embodiments, the length of the effector protein is not greater than 750 linked amino acid residues. In some embodiments, the length of the effector protein is not greater than 800 linked amino acid residues. In some embodiments, the length of the effector protein is not greater than 850 linked amino acid residues. In some embodiments, the length of the effector protein is not greater than 900 linked amino acid residues. In some embodiments, the length of the effector protein is about 350 to about 450 linked amino acid residues. In some embodiments, the length of the effector protein is about 375 to about 475 linked amino acid residues. In some embodiments, the length of the effector protein is about 400 to about 450 linked amino acid residues. In some embodiments, the length of the effector protein is about 400 to about 500 linked amino acid residues. In some embodiments, the length of the effector protein is about 350 to about 400, about 400 to about 450, about 450 to about 550, about 400 to about 420, about 420 to about 440, about 440 to about 460, about 460 to about 480, about 480 to about 500, about 500 to about 520, about 520 to about 540, about 540 to about 560, about 560 to about 580, about 580 to about 600, about 600 to about 620, about 620 to about 640, about 640 to about 660, about 660 to about 680, about 680 to about 700 linked amino acids. In some embodiments, the length of the effector protein is at least 200, at least 225, at least 250, at least 275 at least 300, at least 325, at least 350, at least 375, at least 400, at least 425, at least 450, at least 475, at least 500, at least 525, at least 550, at least 575, at least 600, at least 625, at least 650, at least 675, at least 700, at least 725, at least 750, at least 775 linked amino acids. In some embodiments, the length of the effector protein is about 700 to about 800 linked amino acid residues. In some embodiments, the length of the effector protein is about 750 to about 850, about 700 to about 820, about 720 to about 840, about 740 to about 860, about 760 to about 880, about 780 to about 900, about 800 to about 920, about 820 to about 940, about 840 to about 960, about 860 to about 980, about 880 to about 1000, about 900 to about 1020, about 920 to about 1040, about 940 to about 1060, about 960 to about 1080, about 980 to about 1100 linked amino acids. In some embodiments, the length of the effector protein is about 1000 to about 1100 linked amino acid residues. In some embodiments, the length of the effector protein is about 1050 to about 1150, about 1000 to about 1120, about 1020 to about 1140, about 1040 to about 1160, about 1060 to about 1180, about 1080 to about 1200, about 1100 to about 1220, about 1120 to about 1240, about 1140 to about 1260, about 1160 to about 1280, about 1180 to about 1300, about 1200 to about 1320, about 1220 to about 1340, about 1240 to about 1360, about 1260 to about 1380, about 1280 to about 1400 linked amino acids. In some embodiments, the length of the effector protein is about 1300 to about 1400 linked amino acid residues. In some embodiments, the length of the effector protein is about 1350 to about 1450, about 1300 to about 1420, about 1320 to about 1440, about 1340 to about 1460, about 1360 to about 1480, about 1380 to about 1500, about 1400 to about 1520, about 1420 to about 1540, about 1440 to about 1560, about 1460 to about 1580, about 1480 to about 1600, about 1500 to about 1620, about 1520 to about 1640, about 1540 to about 1660, about 1560 to about 1680, about 1580 to about 1700 linked amino acids.

TABLE 1 provides illustrative amino acid sequences of effector proteins, guide sequences, and PAM sequences that are useful in the compositions, systems and methods described herein. With regards to the PAM sequences: N is any nucleotide, V is A, C, or G; B is C, G, or T; and S is G or C.

In some embodiments, systems and compositions comprise an effector protein and a guide nucleic acid, wherein the effector protein comprises an amino acid sequence that is at least 70%, at least 75%, at least 80%, at least 85%, at least 90%, at least 95%, at least 97%, at least 98%, at least 99%, or 100% identical to SEQ ID NO: 346, and the guide nucleic acid comprises a nucleotide sequence that is at least 70%, at least 75%, at least 80%, at least 85%, at least 90%, at least 95%, at least 97%, at least 98%, at least 99%, or 100% identical to the sequence: ACAGCUUAUUUGGAAGCUGAAAUGUGAGGUUUAUAACACUCACAAGAAUCCUGA AAAAGGAUGCCAAAC (SEQ ID NO: 348). In some embodiments, the guide nucleic acid comprises a nucleotide sequence that is at least 70%, at least 75%, at least 80%, at least 85%, at least 90%, at least 95%, at least 97%, at least 98%, at least 99%, or 100% identical to SEQ ID NO: 347.

In some embodiments, systems and compositions comprise an effector protein and a guide nucleic acid, wherein the effector protein comprises an amino acid sequence that is at least 70%, at least 75%, at least 80%, at least 85%, at least 90%, at least 95%, at least 97%, at least 98%, at least 99%, or 100% identical to SEQ ID NO: 72, and the guide nucleic acid comprises a nucleotide sequence that is at least 70%, at least 75%, at least 80%, at least 85%, at least 90%, at least 95%, at least 97%, at least 98%, at least 99%, or 100% identical to the sequence: UGGGGCAGUUGGUUGCCCUUAGCCUGAGGCAUUUAUUGCACUCGGGAAGUACCA UUUCUCAGAAAAAGGAUGCCAAAC (SEQ ID NO: 349). In some embodiments, the guide nucleic acid comprises a nucleotide sequence that is at least 70%, at least 75%, at least 80%, at least 85%, at least 90%, at least 95%, at least 97%, at least 98%, at least 99%, or 100% identical to a sequence selected from SEQ ID NO: 200-219.

In certain embodiments, compositions, systems, and methods provided herein comprise an effector protein and an engineered guide nucleic acid, wherein the effector protein comprises an amino acid sequence that is at least 65%, at least 70%, at least 75%, at least 80%, at least 85%, at least 90%, at least 95%, at least 98%, at least 99%, or 100% identical to any one of the sequences as set forth in TABLE 1.

In some instances, effector proteins differ from a sequence in TABLE 1 by one or more amino acids. In some instances, effector proteins differ from a sequence in TABLE 1 by 1 amino acid, 2 amino acids, 3 amino acids, 4, amino acids, 5 amino acids, 6 amino acids, 7 amino acids, 8 amino acids, 9 amino acids or 10 amino acids. In some embodiments up to 15 amino acids are modified. In some embodiments up to 20 amino acids are modified. In some embodiments up to 25 amino acids are modified.

In some embodiments the modifications are conservative substitutions relative to the effector protein sequence in TABLE 1. In some embodiments, the amino acids that differ from the effector proteins are non-conservative substitutions relative to the effector protein sequence in TABLE 1. In some embodiments, a mutation may affect the catalytic activity of the effector protein and results in a catalytically reduced or catalytically inactive mutant. In some embodiments, a mutation can result in the effector protein having nickase activity or increased nickase activity. In some embodiments, a mutation can result in the effector protein having reduced or no nuclease activity but gaining nickase activity.

In certain embodiments, compositions, systems and methods described herein provided herein comprise an effector protein, wherein the amino acid sequence of the effector protein comprises at least about 200, at least about 220, at least about 240, at least about 260, at least about 280, at least about 300, at least about 320, at least about 340, at least about 360, at least about 380, at least about 400, at least about 450, at least about 500, at least about 550, at least about 600, at least about 650, at least about 700, at least about 750, at least about 800, at least about 850, at least about 900, at least about 950, at least about 1000, at least about 1050, at least about 1100, at least about 1150, at least about 1200, at least about 1250, or at least about 1300 contiguous amino acids of any one of the effector sequences recited in TABLE 1.

TABLE 1 provides sequences for exemplary CasPhi (CasΦ) proteins which may process their associated crRNA array and cleave the array into individual crRNAs. Engineered gRNAs in which extra nucleotides are added to the sequence (i.e., RT template) on the 5′ end, may be cleaved off by a CasPhi. This contrasts with Cas9, which does not process its own crRNA array and therefore is not subject to the same issue.

### Protospacer Adjacent Motif (PAM)

Effector proteins of the present disclosure, dimers thereof, and multimeric complexes thereof, may cleave or nick a target nucleic acid within or near a protospacer adjacent motif (PAM) sequence of the target nucleic acid. In some embodiments, cleavage occurs within 1, 2, 3, 4, 5, 6, 7, 8, 9, 10, 11, 12, 13, 14, 15, 16, 17, 18, 19, 20, 21, 22, 23, 24, or 25 nucleotides of a 5′ or 3′ terminus of a PAM sequence. In some embodiments, a target nucleic acid may comprise a PAM sequence adjacent to a target sequence that is complementary to a guide nucleic acid spacer region. PAMs in compositions, systems, and methods herein are further described throughout the application. In some embodiments the PAM is NTTN where N can be any amino acid.

In some embodiments, a target nucleic acid comprises a target sequence that is adjacent to a PAM sequence, wherein the PAM sequence comprises any one of the PAM sequences as set forth in TABLE 1. In some embodiments, systems, compositions, and/or methods described herein comprise a target nucleic acid comprising a target sequence that is adjacent to a PAM sequence, wherein the PAM sequence comprises any one of the PAM sequences as set forth in TABLE 1.

### RNA-Dependent DNA Polymerases (RDDP)

In some embodiments, the present disclosure provides for systems, compositions, and methods comprising an RNA-dependent DNA polymerase (RDDP) or a use thereof. Herein, RDDPs are enzymes that are capable of generating a DNA polynucleotide from an RNA template polynucleotide. In some embodiments, the RDDP is a reverse transcriptase. Herein, the term RDDP encompasses functional domains thereof. In some embodiments, the functional domain is a polymerase domain, an RNAse domain, or a combination thereof.

In some embodiments, the RDDP comprises less than 800 amino acids. In some embodiments, the RDDP comprises less than 800, less than 700, less than 600, less than 500, less than 400, less than 300 amino acids. In some embodiments, the RDDP comprises at least 200, at least 220, at least 240, at least 260, at least 280, or at least 300 amino acids. In some embodiments, the RDDP comprises at least 277 amino acids. In some embodiments, the RDDP comprises 200 to 800 amino acids. In some embodiments, the RDDP comprises 277 to 800 amino acids. In some embodiments, the RDDP comprises 300 to 800, 400 to 800, 500 to 800, or 600 to 800 amino acids. In some embodiments, the RDDP comprises 250 to 750, 250 to 700, 250 to 650, 250 to 600, 250 to 550, 250 to 500, 250 to 450, 250 to 400, 250 to 350, 275 to 750, 275 to 700, 275 to 650, 275 to 600, 275 to 550, 275 to 500, 275 to 450, 275 to 400, 275 to 350, 300 to 750, 300 to 700, 300 to 650, 300 to 600, 300 to 550, 300 to 500, 300 to 450, 300 to 400, or 300 to 350 amino acids.

In some embodiments, the length of the RDDP is less than 800 amino acids. In some embodiments, the length of the RDDP is less than 800, less than 700, less than 600, less than 500, less than 400, less than 300 linked amino acids. In some embodiments, the length of the RDDP is at least 200, at least 220, at least 240, at least 260, at least 280, or at least 300 linked amino acids. In some embodiments, the length of the RDDP is at least 277 linked amino acids. In some embodiments, the length of the RDDP is 200 to 800 linked amino acids. In some embodiments, the length of the RDDP is 277 to 800 linked amino acids. In some embodiments, the length of the RDDP is 300 to 800, 400 to 800, 500 to 800, or 600 to 800 linked amino acids. In some embodiments, the length of the RDDP is 250 to 750, 250 to 700, 250 to 650, 250 to 600, 250 to 550, 250 to 500, 250 to 450, 250 to 400, 250 to 350, 275 to 750, 275 to 700, 275 to 650, 275 to 600, 275 to 550, 275 to 500, 275 to 450, 275 to 400, 275 to 350, 300 to 750, 300 to 700, 300 to 650, 300 to 600, 300 to 550, 300 to 500, 300 to 450, 300 to 400, or 300 to 350 linked amino acids.

In some embodiments, the RDDP, when used in combination with an effector protein described herein, demonstrates an editing efficiency of at least 1%. In some embodiments, the RDDP, when used in combination with an effector protein described herein, demonstrates an editing efficiency of at least 2%, 3%, 4%, 5%, 6%, 7%, 8%, 9%, or at least 10%. In some embodiments, the RDDP, when used in combination with an effector protein described herein, demonstrates an editing efficiency that is greater than the editing efficiency observed with a Moloney murine leukemia virus (M-MLV) reverse transcriptase (e.g., SEQ ID NO: 531 or 532). In some embodiments, the RDDP, when used in combination with an effector protein described herein, demonstrates an editing efficiency that is at least 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, or at least 10-fold greater than the editing efficiency observed with an M-MLV reverse transcriptase.

In some embodiments, the RDDP comprises an amino acid sequence that it at least 90%, 91%, 92%, 93%, 94%, 95%, 96%, 97%, 98%, or 99% identical to one of SEQ ID NOs: 485-530 or 545-567. In some embodiments, the RDDP comprises or consists of an amino sequence selected from SEQ ID NOs: 485-530 or 545-567.

In some embodiments, the RDDP comprises an amino acid sequence that it at least 90%, 91%, 92%, 93%, 94%, 95%, 96%, 97%, 98%, or 99% identical to SEQ ID NO: 496. In some embodiments, the RDDP comprises or consists of the amino sequence of SEQ ID NO: 496. In some embodiments, the RDDP comprises an amino acid sequence that it at least 90%, 91%, 92%, 93%, 94%, 95%, 96%, 97%, 98%, or 99% identical to SEQ ID NO: 498. In some embodiments, the RDDP comprises or consists of the amino sequence of SEQ ID NO: 498. In some embodiments, the RDDP comprises an amino acid sequence that it at least 90%, 91%, 92%, 93%, 94%, 95%, 96%, 97%, 98%, or 99% identical to SEQ ID NO: 504. In some embodiments, the RDDP comprises or consists of the amino sequence of SEQ ID NO: 504. In some embodiments, the RDDP comprises an amino acid sequence that it at least 90%, 91%, 92%, 93%, 94%, 95%, 96%, 97%, 98%, or 99% identical to SEQ ID NO: 506. In some embodiments, the RDDP comprises or consists of the amino sequence of SEQ ID NO: 506. In some embodiments, the RDDP comprises an amino acid sequence that it at least 90%, 91%, 92%, 93%, 94%, 95%, 96%, 97%, 98%, or 99% identical to SEQ ID NO: 514. In some embodiments, the RDDP comprises or consists of the amino sequence of SEQ ID NO: 514.

In some embodiments, the present disclosure provides a polynucleotide encoding an RDDP described herein. In some embodiments, the present disclosure provides a polynucleotide encoding an RDDP comprising an amino acid sequence that it at least 90%, 91%, 92%, 93%, 94%, 95%, 96%, 97%, 98%, or 99% identical to one of SEQ ID NOs: 485-530 or 545-567. In some embodiments, the polynucleotide encodes an RDDP comprising or consisting of an amino sequence selected from SEQ ID NOs: 485-530 or 545-567. Exemplary RDDP sequences are provided in TABLE 3 below.

In some instances, RDDPs comprise at least 200, at least 225, at least 250, at least 275 at least 300, at least 325, at least 350, at least 375, at least 400, at least 425, at least 450, at least 475, at least 500, at least 525, at least 550, at least 575, at least 600, at least 625, at least 650, at least 675, at least 700, at least 725, at least 750, at least 775 contiguous amino acids of a sequence selected from SEQ ID NOs: 485-530 or 545-567.

### Engineered Proteins

In another example, any of the protein sequences herein may be codon optimized. In some embodiments, effector protein described herein are encoded by a codon optimized nucleic acid. In some embodiments, a nucleic acid sequence encoding an effector protein described herein, is codon optimized. This type of optimization can entail a mutation of an effector protein encoding nucleotide sequence to mimic the codon preferences of the intended host organism or cell while encoding the same polypeptide. Thus, the codons can be changed, but the encoded protein remains unchanged. For example, if the intended target cell was a human cell, a human codon-optimized effector protein-encoding nucleotide sequence could be used. As another non-limiting example, if the intended host cell were a mouse cell, then a mouse codon-optimized effector protein-encoding nucleotide sequence could be generated. As another non-limiting example, if the intended host cell were a eukaryotic cell, then a eukaryote codon-optimized effector protein nucleotide sequence could be generated. As another non-limiting example, if the intended host cell were a prokaryotic cell, then a prokaryote codon-optimized effector protein-encoding nucleotide sequence could be generated. Codon usage tables are readily available, for example, at the “Codon Usage Database” available at www.kazusa.or.jp/codon. Accordingly, in some embodiments, effector proteins described herein may be codon optimized for expression in a specific cell, for example, a bacterial cell, a plant cell, a eukaryotic cell, an animal cell, a mammalian cell, or a human cell. In some embodiments, the effector protein is codon optimized for a human cell.

It is understood that when describing coding sequences of polypeptides described herein, said coding sequences do not necessarily require a codon encoding a N-terminal Methionine (M) or a Valine (V) as described for the effector proteins described herein. One skilled in the art would understand that a start codon could be replaced or substituted with a start codon that encodes for an amino acid residue sufficient for initiating translation in a host cell. In some embodiments, when a modifying heterologous peptide, such as a fusion partner protein, protein tag or NLS, is located at the N terminus of the effector protein, a start codon for the heterologous peptide serves as a start codon for the effector protein as well. Thus, the natural start codon encoding an amino acid residue sufficient for initiating translation (e.g., Methionine (M) or a Valine (V)) of the effector protein may be removed or absent.

In some embodiments, the RDDP and/or effector proteins described herein comprise one or more amino acid substitutions as compared to a naturally occurring RDDP and/or effector protein. In some embodiments, the amino acid substitution is a conservative amino acid substitution. In general, a conservative amino acid substitution is the substitution of an amino acid residue for another amino acid residue with similar chemical properties (e.g., size, charge, or polarity). Conservative substitutions may be made by exchanging an amino acid from one of the groups listed below (group 1 to 6) for another amino acid of the same group.

Amino acid residues may be divided into groups based on common side chain properties, as follows: (group 1) hydrophobic: norleucine, methionine (Met), Alanine (Ala), Valine (Val), neutral hydrophilic: Cysteine (Cys), Serine (Ser), Leucine (Leu), Isoleucine (Ile); (group 2) Threonine (Thr), Asparagine (Asn), Glutamine (Gln); (group 3) acidic: Aspartic acid (Asp), Glutamic acid (Glu); (group 4) basic: Histidine (His), Lysine (Lys), Arginine (Arg); (group 5) residues that influence chain orientation: Glycine (Gly), Proline (Pro); and (group 6) aromatic: Tryptophan (Trp), Tyrosine (Tyr), Phenylalanine (Phe). The substitution of one amino acid with another amino acid in a same group listed above may be considered a conservative amino acid substitution. The substitution of one amino acid with another amino acid in a different group listed above may be considered a non-conservative amino acid substitution.

In some embodiments, the effector protein is an engineered effector protein and comprises an amino acid sequence that is at least 90%, at least 95%, at least 97%, at least 98%, or at least 99% identical to SEQ ID NO: 346, wherein the polypeptide comprises at least one amino acid substitution relative to SEQ ID NO: 346, wherein the amino acid substitution is at a position selected from K58, I80, T84, K105, N193, C202, S209, G210, A218, D220, E225, C246, N286, M295, M298, A306, Y315, Q360, and a combination thereof. In some embodiments, the polypeptide comprises an amino acid sequence that is 100% identical to SEQ ID NO: 346, with the exception of at least one amino acid substitution relative to SEQ ID NO: 346, wherein the amino acid substitution is a position selected from K58, I80, T84, K105, N193, C202, S209, G210, A218. D220, E225, C246, N286, M295, M298, A306, Y315, Q360, and a combination thereof. In some embodiments, the amino acid substitution is selected from K58X, 180X, T84X, K105X, N193X, C202X, S209X, G210X, A218X, D220X, E225X, C246X, N286X, M295X, M298X, A306X, Y315X, and Q360X, wherein X is selected from R, K, and H.

In some embodiments, the effector protein is an engineered effector protein and comprises an amino acid sequence that is at least 90%, at least 95%, at least 97%, at least 98%, or at least 99% identical to SEQ ID NO: 346, wherein the polypeptide comprises at least one amino acid substitution relative to SEQ ID NO: 346, wherein the amino acid substitution is selected from I80R, T84R, K105R, C202R, G210R, A218R, D220R, E225R, C246R, Q360R, 180K, T84K, G210K, N193K, C202K, A218K, D220K, E225K, C246K, N286K, A306K, Q360K, 180H, T84H, K105H, G210H, C202H, A218H, D220H, E225H, C246H, Q360H, K58W, S209F, M295W, M298L, Y315M, D220R/A306K and D220R/K250N and a combination thereof. In some embodiments, the polypeptide comprises an amino acid sequence that is 100% identical to SEQ ID NO: 346, with the exception of at least one amino acid substitution relative to SEQ ID NO: 346, wherein the amino acid substitution is selected from 180R, T84R, K105R, C202R, G210R, A218R, D220R, E225R, C246R, Q360R, 180K, T84K, G210K, N193K, C202K, A218K, D220K, E225K, C246K, N286K, A306K, Q360K, 180H, T84H, K105H, G210H, C202H, A218H, D220H, E225H, C246H, Q360H, K58W, S209F, M295W, M298L, Y315M, D220R/A306K and D220R/K250N and a combination thereof. In some aspects, these engineered effector proteins demonstrate enhanced nuclease activity relative to the wild-type effector protein.

In some embodiments, the effector protein is an engineered effector protein and comprises an amino acid sequence that is at least 90%, at least 95%, at least 97%, at least 98%, or at least 99% identical to SEQ ID NO: 346, wherein the polypeptide comprises at least one amino acid substitution relative to SEQ ID NO: 346, wherein the amino acid substitution is selected from D237A. D418A, D418N, E335A, and E335Q, and a combination thereof. In some embodiments, the polypeptide comprises an amino acid sequence that is 100% identical to SEQ ID NO: 346, with the exception of at least one amino acid substitution relative to SEQ ID NO: 346, wherein the amino acid substitution is selected from D237A, D418A, D418N, E335A, and E335Q, and a combination thereof. In some aspects, these engineered effector proteins demonstrate reduced or abolished nuclease activity relative to the wild-type effector protein. TABLE 1.1 provides the exemplary amino acid alterations relative to SEQ ID NO: 346 useful in compositions, systems, and methods described herein.

In some embodiments, the effector protein is an engineered effector protein and comprises an amino acid sequence that is at least 90%, at least 95%, at least 97%, at least 98%, or at least 99% identical to SEQ ID NO: 12, wherein the polypeptide comprises at least one amino acid substitution relative to SEQ ID NO: 12, wherein the amino acid substitution is at a position selected from 12, T5, K15, R18, H20, S21, L26, N30, E33, E34, A35, K37, K38, R41, N43, Q54, Q79R, K92E, K99R, S108, E109, H110, G111, D113, T114, P116, K118, E119, A121, N132, K135, Q138, V139, N148, L149, E157, E164, E166, E170, Y180, L182, Q183, K184, S186, K189, S196, S198, K200, 1203, S205, K206, Y207, H208, N209, Y220, S223, E258, K281, K348, N355, S362, N406, K435, I471, I489, Y490, F491, D495, K496, K498, K500, D501, V502, K504, S505, D506, V521, N568, S579, Q612, S638, F701, P707, and a combination thereof. In some embodiments, the polypeptide comprises an amino acid sequence that is 100% identical to SEQ ID NO: 12, with the exception of at least one amino acid substitution relative to SEQ ID NO: 12, wherein the amino acid substitution is at a position selected from 12, T5, K15, R18, H20, S21, L26, N30, E33, E34, A35, K37, K38, R41, N43, Q54, Q79R, K92E, K99R, S108, E109, H110. G111, D113, T114, P116, K118, E119, A121, N132, K135, Q138, V139, N148, L149, E157, E164, E166, E170, Y180, L182, Q183, K184, S186, K189. S196, S198, K200, 1203, S205, K206, Y207, H208, N209, Y220, S223, E258, K281, K348, N355, S362, N406, K435, I471, 1489, Y490, F491, D495, K496, K498, K500, D501, V502, K504, S505, D506, V521, N568, S579, Q612, S638, F701, P707, and a combination thereof. In some embodiments, the amino acid substitution is selected from I2X, T5X, K15X, R18X, H20X, S21X, L26X, N30X, E33X, E34X, A35X, K37X, K38X, R41X, N43X, Q54X, Q79RX, K92EX, K99RX, S108X, E109X, H110X, G111X, D113X, T114X, P116X, K118X, E119X, A121X, N132X, K135X, Q138X, V139X, N148X, L149X, E157X, E164X, E166X, E170X, Y180X, L182X, Q183X, K184X, S186X, K189X, S196X, S198X, K200X, I203X, S205X, K206X, Y207X, H208X, N209X. Y220X, S223X, E258X. K281X, K348X, N355X. S362X, N406X, K435X, 1471X, 1489X, Y490X, F491X, D495X, K496X, K498X, K500X, D501X. V502X, K504X, S505X, D506X, V521X, N568X, S579X. Q612X, S638X, F701X, P707X, wherein X is selected from R, K, and H.

In some embodiments, the effector protein is an engineered effector protein and comprises an amino acid sequence that is at least 90%, at least 95%, at least 97%, at least 98%, or at least 99% identical to SEQ ID NO: 12, wherein the polypeptide comprises at least one amino acid substitution relative to SEQ ID NO: 12 wherein the amino acid substitution is selected from T5R. L26R, L26K, A121Q, V139R, S198R, S223P, E258K, I471T, S579R, F701R, P707R, K189P, S638K, Q54R, Q79R, Y220S, N406K, E119S, K92E, K435Q, N568D, and V521T, and a combination thereof. In some embodiments, the polypeptide comprises an amino acid sequence that is 100% identical to SEQ ID NO: 12, with the exception of at least one amino acid substitution relative to SEQ ID NO: 12, wherein the amino acid substitution is selected from T5R. L26R. L26K. A121Q, V139R, S198R, S223P, E258K, I471T, S579R, F701R, P707R, K189P, S638K, Q54R, Q79R, Y220S, N406K, E119S, K92E, K435Q, N568D, and V521T, and a combination thereof. In some aspects, these engineered effector proteins demonstrate enhanced nuclease activity relative to the wild-type effector protein.

In some embodiments, the effector protein is an engineered effector protein and comprises an amino acid sequence that is at least 90%, at least 95%, at least 97%, at least 98%, or at least 99% identical to SEQ ID NO: 12, wherein the polypeptide comprises at least one amino acid substitution relative to SEQ ID NO: 12 wherein the amino acid substitution is selected from L26K/A121Q, L26R/A121Q, K99R/L149R, K99R/N148R, L149R/H208R, S362R/L26R L26R/N148R, L26R/H208R, N30R/N148R, L26R/K99R, L26R/P707R, L26R/L149R, L26R/N30R, L26R/N355R, L26R/K281R, L26R/S108R, L26R/K348R, T5R/V139R, I2R/V139R, K99R/S186R, L26R/A673G, L26R/Q674R, S579R/L26K, F701R/E258K, T5R/L26K, L26R/K435Q, L26R/G685R, L26R/Q674K, L26R/P699R, L26R/T70E, L26R/Q232R, L26R/T252R, L26R/P679R, L26R/E83K, L26R/E73P, L26R/K248E, L26R, T5R/S223P, S579R/S223P, L26R/S223P, T5R/A121Q, L26R/A696R, S198R/I471T, L26R/N153R, L26R/E682R, L26R/D703R, Q612R/L26K, L26R/I471T, K348R/L26K, S579R/I471T, L26R/V228R, T5R/S638K, S579R/K189P, S579R/E258K, L26R/K260R, L26R/S638K, S579R/Y220S, T5R/I471T, L26R/F233R, L26R/V521T, F701R/A121Q, L26R/G361R, S198R/E258K, L26R/S472R, T5R/Y220S, L26R/A150K, L26R/S684R, L26R/E157R, L26R/K248R, F701R/L26K, S198R/N406K, S198R/Y220S, S198R/S638K, S198R/V521T, S579R/A121Q, K348R/Y220S, S198R/K189P, L26R/E242R, L26R/K678R, T5R/N406K, L26R/I158K, T5R/V521T, L26R/N259R, L26R/K257R, L26R/K256R, T5R/K189P, L26R/C405R, S579R/V521T, S579R/N406K, T5R/K92E, T5R/E258K, L26R/197R, S579R/S638K, T5R/K435Q, F701R/S638K, L26R/L236R, F701R/I471T, Q612R/S223P, F701R/S223P, S198R/E119S, S579R/K92E, L26R/E715R, Q612R/I471T, F701R/Y220S, S198R/S223P, and L26R/K266R, and a combination thereof. In some embodiments, the polypeptide comprises an amino acid sequence that is 100% identical to SEQ ID NO: 12, with the exception of at least one amino acid substitution relative to SEQ ID NO: 12, wherein the amino acid substitution is selected from L26K/A121Q, L26R/A121Q, K99R/L149R, K99R/N148R, L149R/H208R, S362R/L26R L26R/N148R, L26R/H208R, N30R/N148R, L26R/K99R, L26R/P707R. L26R/L149R, L26R/N30R, L26R/N355R, L26R/K281R, L26R/S108R, L26R/K348R, T5R/V139R, 12R/V139R, K99R/S186R, L26R/A673G, L26R/Q674R, S579R/L26K, F701R/E258K, T5R/L26K, L26R/K435Q, L26R/G685R, L26R/Q674K, L26R/P699R, L26R/T70E, L26R/Q232R, L26R/T252R, L26R/P679R, L26R/E83K, L26R/E73P, L26R/K248E, L26R, T5R/S223P, S579R/S223P, L26R/S223P, T5R/A121Q, L26R/A696R, S198R/I471T, L26R/N153R, L26R/E682R, L26R/D703R, Q612R/L26K, L26R/I471T, K348R/L26K, S579R/I471T, L26R/V228R, T5R/S638K, S579R/K189P, S579R/E258K, L26R/K260R, L26R/S638K, S579R/Y220S, T5R/I471T, L26R/F233R, L26R/V521T, F701R/A121Q, L26R/G361R, S198R/E258K, L26R/S472R, T5R/Y220S, L26R/A150K, L26R/S684R, L26R/E157R, L26R/K248R, F701R/L26K, S198R/N406K, S198R/Y220S, S198R/S638K, S198R/V521T, S579R/A121Q, K348R/Y220S, S198R/K189P, L26R/E242R, L26R/K678R, T5R/N406K, L26R/I158K, T5R/V521T, L26R/N259R, L26R/K257R, L26R/K256R, T5R/K189P, L26R/C405R, S579R/V521T, S579R/N406K, T5R/K92E, T5R/E258K, L26R/197R, S579R/S638K, T5R/K435Q, F701R/S638K, L26R/L236R, F701R/I471T, Q612R/S223P, F701R/S223P, S198R/E119S, S579R/K92E, L26R/E715R, Q612R/I471T, F701R/Y220S, S198R/S223P, and L26R/K266R, and a combination thereof. In some aspects, these engineered effector proteins demonstrate enhanced nuclease activity relative to the wild-type effector protein.

In some embodiments, the effector protein is an engineered effector protein and comprises an amino acid sequence that is at least 90%, at least 95%, at least 97%, at least 98%, or at least 99% identical to SEQ ID NO: 12, wherein the polypeptide comprises at least one amino acid substitution relative to SEQ ID NO: 34612 wherein the amino acid substitution is selected from E157A, E164A, E164L, E166A, E166I, E170A, 1489A, 1489S, Y490S, Y490A, F491A, F491S, F491G, D495G, D495R, D495K, K496A, K496S, K498A, K498S, K500A, K500S, D501R, D501G, D501K, V502A, V502S, K504A, K504S, S505R, D506A, and a combination thereof. In some embodiments, the polypeptide comprises an amino acid sequence that is 100% identical to SEQ ID NO: 12, with the exception of at least one amino acid substitution relative to SEQ ID NO: 12, wherein the amino acid substitution is selected from E157A, E164A, E164L, E166A. E166I, E170A, 1489A, 1489S, Y490S, Y490A, F491A, F491S, F491G, D495G, D495R, D495K, K496A, K496S, K498A, K498S, K500A, K500S, D501R, D501G, D501K, V502A, V502S, K504A, K504S, S505R, D506A, and a combination thereof. In some embodiments, these engineered effector proteins comprise a nickase activity.

In some embodiments, the effector protein is an engineered effector protein and comprises an amino acid sequence that is at least 90%, at least 95%, at least 97%, at least 98%, or at least 99% identical to SEQ ID NO: 12, wherein amino acids S478-S505 have been deleted. In some embodiments, the effector protein is an engineered effector protein that is at least 90%, at least 95%, at least 97%, at least 98%, or at least 99% identical to SEQ ID NO: 12, wherein amino acids S478-S505 have been deleted and with replaced SDLYIERGGDPRDVHQQVETKPKGKRKSEIRILKIR (SEQ ID NO: 580) or SDYIVDHGGDPEKVFFETKSKKDKTKRYKRR (SEQ ID NO: 581). In some embodiments, the effector protein is an engineered effector protein and comprises an amino acid sequence that is at least 90%, at least 95%, at least 97%, at least 98%, at least 99% identical, or is 100% identical to SEQ ID NO: 578 In some embodiments, the effector protein is an engineered effector protein and comprises an amino acid sequence that is at least 90%, at least 95%, at least 97%, at least 98%, at least 99% identical, or is 100% identical to SEQ ID NO: 579.

In some embodiments, the effector protein is an engineered effector protein and comprises an amino acid sequence that is at least 90%, at least 95%, at least 97%, at least 98%, or at least 99% identical to SEQ ID NO: 12, wherein the polypeptide comprises at least one amino acid substitution relative to SEQ ID NO: 12 wherein the amino acid substitution is selected from D369A. D369N. D658A. D658N. E567A, E567Q, and a combination thereof. In some embodiments, the polypeptide comprises an amino acid sequence that is 100% identical to SEQ ID NO: 12, with the exception of at least one amino acid substitution relative to SEQ ID NO: 12, wherein the amino acid substitution is selected from D369A, D369N, D658A, D658N, E567A. E567Q, and a combination thereof. In some aspects, these engineered effector proteins demonstrate reduced or abolished nuclease activity relative to the wild-type effector protein. TABLE 1.1 provides the exemplary amino acid alterations relative to SEQ ID NO: 12 useful in compositions, systems, and methods described herein.

In some embodiments, the RDDP is an engineered RDDP and comprises an amino acid sequence that it at least 90%, 91%, 92%, 93%, 94%, 95%, 96%, 97%, 98%, or 99% identical to one of SEQ ID NOs: 568-577. In some embodiments, the RDDP comprises or consists of an amino sequence selected from SEQ ID NOs: 568-577.

In some embodiments, the present disclosure provides a polynucleotide encoding an RDDP described herein. In some embodiments, the present disclosure provides a polynucleotide encoding an RDDP comprising an amino acid sequence that it at least 90%, 91%, 92%, 93%, 94%, 95%, 96%, 97%, 98%, or 99% identical to one of SEQ ID NOs: 568-577. In some embodiments, the polynucleotide encodes an RDDP comprising or consisting of an amino sequence selected from SEQ ID NOs: 568-577.

In some instances, RDDPs comprise at least 200, at least 225, at least 250, at least 275 at least 300, at least 325, at least 350, at least 375, at least 400, at least 425, at least 450, at least 475, at least 500, at least 525, at least 550, at least 575, at least 600, at least 625, at least 650, at least 675, at least 700, at least 725, at least 750, at least 775 contiguous amino acids of a sequence selected from SEQ ID NOs: 568-577.

Exemplary engineered RDDPs are provided in TABLE 4 below. For each engineered RDDP, the parental RDDP is listed followed by the amino acid mutations in parentheses. For example, 2691319 (D12R-D72R-N195R) refers to an engineered RDDP based on 2691319 (SEQ ID NO: 496) and comprising the mutations D12R, D72R, and N195R.

Engineered effector proteins may provide enhanced catalytic activity (e.g., nuclease or nickase activity) as compared to a naturally occurring nuclease or nickase. Engineered effector proteins may provide enhance nucleic acid binding activity, e.g., enhanced binding of a guide nucleic acid and/or target nucleic acid, and/or may demonstrate a stronger affinity for a target nucleic acid sequence. Without wishing to be bound by theory, substation of positively charged amino acids is thought to increase the interaction between the effector proteins and/or RDDPs and the negatively charged target nucleic acid sequences. By way of non-limiting example, some engineered proteins exhibit optimal activity at lower salinity and viscosity than the protoplasm of their bacterial cell of origin. Also, by way of non-limiting example, bacteria often comprise protoplasmic salt concentrations greater than 250 mM and room temperature intracellular viscosities above 2 centipoise, whereas engineered proteins exhibit optimal activity (e.g., cis-cleavage activity) at salt concentrations below 150 mM and viscosities below 1.5 centipoise. The present disclosure leverages these dependencies by providing engineered proteins in solutions optimized for their activity and stability.

Compositions and systems described herein may comprise an engineered effector protein and/or RDDP in a solution comprising a room temperature viscosity of less than about 15 centipoise, less than about 12 centipoise, less than about 10 centipoise, less than about 8 centipoise, less than about 6 centipoise, less than about 5 centipoise, less than about 4 centipoise, less than about 3 centipoise, less than about 2 centipoise, or less than about 1.5 centipoise.

Compositions and systems may comprise an engineered effector protein and/or RDDP in a solution comprising an ionic strength of less than about 500 mM, less than about 400 mM, less than about 300 mM, less than about 250 mM, less than about 200 mM, less than about 150 mM, less than about 100 mM, less than about 80 mM, less than about 60 mM, or less than about 50 mM. Compositions and systems may comprise an engineered effector protein and/or RDDP and an assay excipient, which may stabilize a reagent or product, prevent aggregation or precipitation, or enhance or stabilize a detectable signal (e.g., a fluorescent signal). Examples of assay excipients include, but are not limited to, saccharides and saccharide derivatives (e.g., sodium carboxymethyl cellulose and cellulose acetate), detergents, glycols, polyols, esters, buffering agents, alginic acid, and organic solvents (e.g., DMSO).

An engineered protein may comprise a modified form of a wild type counterpart protein (e.g., an effector protein and/or RDDP). The modified form of the wild type counterpart may comprise an amino acid change (e.g., deletion, insertion, or substitution) that reduces the nucleic acid-cleaving activity of the effector protein relative to the wild type counterpart. For example, a nuclease domain (e.g., RuvC domain) of an effector protein may be deleted or mutated relative to a wild type counterpart effector protein so that it is no longer functional or comprises reduced nuclease activity. The modified form of the effector protein may have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart. Engineered effector proteins may have no substantial nucleic acid-cleaving activity. An engineered effector protein may be enzymatically inactive or “dead,” also referred to in some instances as a dead protein or a dCas protein. An engineered effector protein may bind to a guide nucleic acid and/or a target nucleic acid but not cleave the target nucleic acid. An enzymatically inactive effector protein may comprise an enzymatically inactive domain (e.g., inactive nuclease domain). Enzymatically inactive may refer to an activity less than 1%, less than 2%, less than 3%, less than 4%, less than 5%, less than 6%, less than 7%, less than 8%, less than 9%, or less than 10% activity compared to the wild-type counterpart. A dead protein may associate with a guide nucleic acid to activate or repress transcription of a target nucleic acid. In some embodiments, the enzymatically inactive protein is fused to a fusion partner protein that confers an alternative activity to an effector protein activity. Such fusion proteins are described herein and throughout. By way of non-limiting example, an alternative activity may be a transcriptional activation, transcription repression, deaminase activity, transposase activity, and recombinase activity. In some embodiments, activity (e.g., nuclease activity) of effector proteins and/or compositions described herein can be measured relative to a WT effector protein or compositions containing the same in a cleavage assay.

### Modulation of Effector Protein Activity

The effector proteins of the present disclosure may have nuclease activity, nickase activity, or no cleavage activity on target nucleic acids. In some embodiments the effector protein of the present disclosure has a combination of the above activities on a target nucleic acid. In some embodiments the cleavage activity of the effector proteins is modulated between nuclease activity and nickase activity on the target nucleic acid. In some embodiments the cleavage activity of the effector protein is modulated between nickase activity and no cleavage activity on the target nucleic acid. In some embodiments the cleavage activity of the effector protein is modulated between nuclease activity and no cleavage activity on the target nucleic acid. In some embodiments the cleavage activity of the effector protein is modulated between nuclease activity, nickase activity, and no cleavage activity on the target nucleic acid. In some embodiments the effector protein has nuclease activity. In some embodiments the effector protein has nickase activity.

In some embodiments the cleavage activity of the effector protein on the target nucleic acid is modulated based on the length of the spacer sequence of a guide nucleic acid. In some embodiments the spacer length confers nuclease activity to the effector protein on the target nucleic acid. In some embodiments the spacer length confers nickase activity to the effector protein on the target nucleic acid. In some embodiments the spacer length confers no cleavage activity to the effector protein on the target nucleic acid. In some embodiments, the spacer length is 10-20 nucleotides. In some embodiments, the spacer length is 11-17 nucleotides. In some embodiments, the spacer length is 14-16 nucleotides. In some embodiments, the spacer length is 10, 12, 13, 14, 15, 16 or 17 nucleotides. In some embodiments, the spacer length is 15 nucleotides.

### Fusion Proteins

In some embodiments, the present disclosure provides a fusion protein comprising an effector protein described herein and an RDDP described herein. In some embodiments, the fusion protein comprises, from N-terminus to C-terminus, an effector protein and an RDDP. In some embodiments, the fusion protein comprises, from N-terminus to C-terminus, an RDDP and an effector protein.

In some embodiments, the fusion protein described herein comprises an effector protein comprising an amino acid sequence that is at least 90%, 91%, 92%, 93%, 94%, 95%, 96%, 97%, 98%, or 99% identical to an amino acid sequence selected from SEQ ID NOs: 1-134 and 346 and an RDDP comprising an amino acid sequence that is at least 90%, 91%, 92%, 93%, 94%, 95%, 96%, 97%, 98%, or 99% identical to an amino acid sequence selected from SEQ ID NOs: 485-530 or 545-577. In some embodiments, the fusion protein described herein comprises an effector protein comprising or consisting of an amino acid sequence selected from SEQ ID NOs: 1-134 and 346 and an RDDP comprising or consisting of an amino acid sequence selected from SEQ ID NOs: 485-530 or 545-577.

In some embodiments, the fusion protein described herein comprises an effector protein comprising an amino acid sequence that is at least 90%, 91%, 92%, 93%, 94%, 95%, 96%, 97%, 98%, or 99% identical to an amino acid sequence selected from SEQ ID NOs: 1-134 and 346 and an RDDP comprising an amino acid sequence that is at least 90%, 91%, 92%, 93%, 94%, 95%, 96%, 97%, 98%, or 99% identical to SEQ ID NO: 496. In some embodiments, the fusion protein described herein comprises an effector protein comprising or consisting of an amino acid sequence selected from SEQ ID NOs: 1-134 and 346 and an RDDP comprising or consisting of the amino acid sequence of SEQ ID NO: 496. In some embodiments, the fusion protein comprises an amino acid sequence that is at least 90%, 91%, 92%, 93%, 94%, 95%, 96%, 97%, 98%, or 99% identical to SEQ ID NO: 533 or 538. In some embodiments, the fusion protein comprises or consists of SEQ ID NO: 533 or 538.

In some embodiments, the fusion protein described herein comprises an effector protein comprising an amino acid sequence that is at least 90%, 91%, 92%, 93%, 94%, 95%, 96%, 97%, 98%, or 99% identical to an amino acid sequence selected from SEQ ID NOs: 1-134 and 346 and an RDDP comprising an amino acid sequence that is at least 90%, 91%, 92%, 93%, 94%, 95%, 96%, 97%, 98%, or 99% identical to SEQ ID NO: 498. In some embodiments, the fusion protein described herein comprises an effector protein comprising or consisting of an amino acid sequence selected from SEQ ID NOs: 1-134 and 346 and an RDDP comprising or consisting of the amino acid sequence of SEQ ID NO: 498. In some embodiments, the fusion protein comprises an amino acid sequence that is at least 90%, 91%, 92%, 93%, 94%, 95%, 96%, 97%, 98%, or 99% identical to SEQ ID NO: 534 or 539. In some embodiments, the fusion protein comprises or consists of SEQ ID NO: 534 or 539.

In some embodiments, the fusion protein described herein comprises an effector protein comprising an amino acid sequence that is at least 90%, 91%, 92%, 93%, 94%, 95%, 96%, 97%, 98%, or 99% identical to an amino acid sequence selected from SEQ ID NOs: 1-134 and 346 and an RDDP comprising an amino acid sequence that is at least 90%, 91%, 92%, 93%, 94%, 95%, 96%, 97%, 98%, or 99% identical to SEQ ID NO: 504. In some embodiments, the fusion protein described herein comprises an effector protein comprising or consisting of an amino acid sequence selected from SEQ ID NOs: 1-134 and 346 and an RDDP comprising or consisting of the amino acid sequence of SEQ ID NO: 504. In some embodiments, the fusion protein comprises an amino acid sequence that is at least 90%, 91%, 92%, 93%, 94%, 95%, 96%, 97%, 98%, or 99% identical to SEQ ID NO: 535 or 540. In some embodiments, the fusion protein comprises or consists of SEQ ID NO: 535 or 540.

In some embodiments, the fusion protein described herein comprises an effector protein comprising an amino acid sequence that is at least 90%, 91%, 92%, 93%, 94%, 95%, 96%, 97%, 98%, or 99% identical to an amino acid sequence selected from SEQ ID NOs: 1-134 and 346 and an RDDP comprising an amino acid sequence that is at least 90%, 91%, 92%, 93%, 94%, 95%, 96%, 97%, 98%, or 99% identical to SEQ ID NO: 506. In some embodiments, the fusion protein described herein comprises an effector protein comprising or consisting of an amino acid sequence selected from SEQ ID NOs: 1-134 and 346 and an RDDP comprising or consisting of the amino acid sequence of SEQ ID NO: 506. In some embodiments, the fusion protein comprises an amino acid sequence that is at least 90%, 91%, 92%, 93%, 94%, 95%, 96%, 97%, 98%, or 99% identical to SEQ ID NO: 536 or 541. In some embodiments, the fusion protein comprises or consists of SEQ ID NO: 536 or 541.

In some embodiments, the fusion protein described herein comprises an effector protein comprising an amino acid sequence that is at least 90%, 91%, 92%, 93%, 94%, 95%, 96%, 97%, 98%, or 99% identical to an amino acid sequence selected from SEQ ID NOs: 1-134 and 346 and an RDDP comprising an amino acid sequence that is at least 90%, 91%, 92%, 93%, 94%, 95%, 96%, 97%, 98%, or 99% identical to SEQ ID NO: 514. In some embodiments, the fusion protein described herein comprises an effector protein comprising or consisting of an amino acid sequence selected from SEQ ID NOs: 1-134 and 346 and an RDDP comprising or consisting of the amino acid sequence of SEQ ID NO: 514. In some embodiments, the fusion protein comprises an amino acid sequence that is at least 90%, 91%, 92%, 93%, 94%, 95%, 96%, 97%, 98%, or 99% identical to SEQ ID NO: 537 or 542. In some embodiments, the fusion protein comprises or consists of SEQ ID NO: 537 or 542.

### Tethers

In some embodiments the effector protein is complexed with all or part of a biological tether or a protein localization sequence. In some embodiments the RDDP is bound to the biological tether or protein localization sequence. In some embodiments the guide RNA is bound to an aptamer that is recognized by the biological tether protein.

In some embodiments, the biological tether or protein localization sequence is MS2, Csy4 or lambda N protein.

In some embodiments, the effector proteins are complexed with a biological tether, and comprises all or part of (e.g., DNA binding domain from) the MS2 coat protein, endoribonuclease Csy4, or the lambda N protein. These proteins can be used to recruit RNA molecules containing a specific stem-loop structure to a locale specified by the Type V effector protein guide RNA targeting sequences. For example, a Type V effector protein variant fused to MS2 coat protein, endoribonuclease Csy4, or lambda N can be used to recruit a long non-coding RNA (lncRNA) such as XIST or HOTAIR; see, e.g., Keryer-Bibens et al., Biol. Cell 100:125-138 (2008), that is linked to the Csy4, MS2 or lambda N binding sequence. Alternatively, the Csy4, MS2 or lambda N protein binding sequence can be linked to another protein, e.g., as described in Keryer-Bibens et al., supra, and the protein can be targeted to the dCpf1 variant binding site using the methods and compositions described herein. In some embodiments, the Csy4 is catalytically inactive.

### Synthesis, Isolation and Assaying

RDDPs, effector proteins and fusion proteins of the present disclosure of the present disclosure may be synthesized, using any suitable method. Additionally, nucleic acids, including mRNA encoding effector proteins and fusion proteins of the present disclosure may be synthesized using suitable methods. RDDPs, effector proteins and fusion proteins of the present disclosure may be produced in vitro or by eukaryotic cells or by prokaryotic cells. Effector proteins can be further processed by unfolding, e.g. heat denaturation, dithiothreitol reduction, etc. and may be further refolded, using any suitable method.

Methods of generating and assaying the RDDPs, effector proteins and fusion proteins described herein are well known to one of skill in the art. Examples of such methods are described in the Examples provided herein. Any of a variety of methods can be used to generate an effector protein disclosed herein. Such methods include, but are not limited to, site-directed mutagenesis, random mutagenesis, combinatorial libraries, and other mutagenesis methods described herein (see, e.g., Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, MD (1999); Gillman et al., Directed Evolution Library Creation: Methods and Protocols (Methods in Molecular Biology) Springer, 2nd ed (2014)). One non-limiting example of a method for preparing an effector protein is to express recombinant nucleic acids encoding the effector protein in a suitable microbial organism, such as a bacterial cell, a yeast cell, or other suitable cell, using methods well known in the art.

In some embodiments, an RDDP, effector protein, and/or fusion protein provided herein is an isolated effector protein. In some embodiments, an RDDP, effector protein, and/or fusion protein described herein can be isolated and purified for use in compositions, systems, and/or methods described herein. Methods described here can include the step of isolating effector proteins described herein. An isolated an RDDP, effector protein, and/or fusion protein provided herein can be isolated by a variety of methods well-known in the art, for example, recombinant expression systems, precipitation, gel filtration, ion-exchange, reverse-phase and affinity chromatography, and the like. Other well-known methods are described in Deutscher et al., Guide to Protein Purification: Methods in Enzymology, Vol. 182, (Academic Press, (1990)). Alternatively, the isolated polypeptides of the present disclosure can be obtained using well-known recombinant methods (see, e.g., Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999)). The methods and conditions for biochemical purification of a polypeptide described herein can be chosen by those skilled in the art, and purification monitored, for example, by a functional assay.

RDDPs, effector proteins, and/or fusion proteins disclosed herein may be covalently linked or attached to a tag, e.g., a purification tag. A purification tag, as used herein, can be an amino acid sequence which can attach or bind with high affinity to a separation substrate and assist in isolating the protein of interest from its environment, which can be its biological source, such as a cell lysate. Attachment of the purification tag can be at the N or C terminus of the effector protein. Furthermore, an amino acid sequence recognized by a protease or a nucleic acid encoding for an amino acid sequence recognized by a protease, such as TEV protease or the HRV3C protease can be inserted between the purification tag and the effector protein, such that biochemical cleavage of the sequence with the protease after initial purification liberates the purification tag. Purification and/or isolation can be through high performance liquid chromatography (HPLC), exclusion chromatography, gel electrophoresis, affinity chromatography, or other purification technique. Examples of purification tags are as described herein.

### Guide Nucleic Acids

The compositions, systems, and methods of the present disclosure may comprise a guide nucleic acid or a use thereof. Unless otherwise indicated, compositions, systems and methods comprising guide nucleic acids or uses thereof, as described herein and throughout, include DNA molecules, such as expression vectors, that encode a guide nucleic acid. Accordingly, compositions, systems, and methods of the present disclosure comprise a guide nucleic acid or a nucleotide sequence encoding the guide nucleic acid.

In general, a guide nucleic acid is a nucleic acid molecule, at least a portion of which may be bound by an effector protein, thereby forming a ribonucleoprotein complex (RNP). Another portion of the guide nucleic acid molecule can comprise a spacer region which is complementary to at least a portion of the target nucleic acid sequence. In some embodiments, the guide nucleic acid imparts activity or sequence selectivity to the effector protein. When complexed with an effector protein, guide nucleic acids can bring the effector protein into proximity of a target nucleic acid. The guide nucleic acid spacer region may hybridize to a target nucleic acid or a portion thereof. In some embodiments, when a guide nucleic acid and an effector protein form an RNP, at least a portion of the RNP binds spacer region, recognizes, and/or hybridizes to a target nucleic acid. Those skilled in the art in reading the below specific examples of guide nucleic acids as used in RNPs described herein, will understand that in some embodiments, a RNP can hybridize, via the spacer region, to one or more target sequences in a target nucleic acid, thereby allowing the RNP to modify and/or recognize a target nucleic acid or sequence contained therein.

A guide nucleic acid, as well as any components thereof (e.g., spacer region, repeat region, linker) may comprise one or more deoxyribonucleotides, ribonucleotides, biochemically or chemically modified nucleotides (e.g., one or more sequence modifications as described herein), and any combinations thereof. A guide nucleic acid may comprise a naturally occurring sequence. A guide nucleic acid may comprise a non-naturally occurring sequence, wherein the sequence of the guide nucleic acid, or any portion thereof, may be different from the sequence of a naturally occurring nucleic acid. The guide nucleic acid may be chemically synthesized or recombinantly produced. Guide nucleic acids and portions thereof may be found in or identified from a CRISPR array present in the genome of a host organism or cell.

Guide nucleic acids, while often being referred to as a guide RNA, may include deoxyribonucleosides, ribonucleosides, chemically modified nucleosides, or any combination thereof.

In some embodiments, the guide nucleic acid comprises a hairpin or stem-loop structure that is recognized by the effector protein. The hairpin or stem-loop structure may comprise a repeat region. In some embodiments, the guide nucleic acid comprises at least a portion of a tracrRNA sequence. In nature, a tracrRNA is a guide nucleic acid that has trans activating activity on a target nucleic acid through a repeat hybridization sequence. In some instances, guide nucleic acids of the instant disclosure do not comprise a repeat hybridization sequence. In some instances, guide nucleic acids of the instant disclosure do not comprise a tracrRNA. In some instances, e.g., systems comprising CasPhi proteins and fusions thereof, the guide nucleic acid does not comprise a tracrRNA sequence but comprises a repeat sequence to which the CasPhi protein binds.

In some embodiments, the guide nucleic acid comprises a repeat region that interacts with the effector protein. The term, “repeat region” may be used interchangeably herein with the term, “repeat sequence.” In some instances, an effector protein interacts with a repeat region. In some instances, an effector protein does not interact with a repeat region. Typically, the repeat region is adjacent to the spacer region. In certain embodiments, the repeat region is followed by the spacer region in the 5′ to 3′ direction. Exemplary repeat region sequences for exemplary effector proteins provided herein are shown in TABLE 1.

### Spacer Sequences

In general, guide nucleic acids comprise a spacer sequence that hybridizes with a target sequence of a target nucleic acid. The term, “spacer sequence,” refers to a region of the guide nucleic acid that hybridizes to a target sequence of a target nucleic acid. The terms “spacer sequence” and “spacer region” are used interchangeably herein and throughout. The spacer sequence may comprise a sequence that is complementary with a target sequence of a target nucleic acid. In some embodiments, the spacer sequence is complementary to the target sequence on the target strand of a dsDNA molecule. In some embodiments, the spacer sequence is complementary to the target sequence on the non-target strand of a dsDNA molecule. The spacer sequence can function to direct the guide nucleic acid to the target nucleic acid for detection and/or modification of the target nucleic acid. The spacer sequence may be complementary to a target sequence that is adjacent to a PAM that is recognizable by an effector protein of interest.

In some embodiments, the spacer sequence is 15-28 linked nucleotides in length. In some embodiments, the spacer sequence is 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides in length. In some embodiments, the spacer sequence is 18-24 linked nucleotides in length. In some embodiments, the spacer sequence is at least 15 linked nucleotides in length. In some embodiments, the spacer sequence is at least 16, 18, 20, or 22 linked nucleotides in length. In some embodiments, the spacer sequence comprises at least 10, 11, 12, 13, 14, 15, 16, 17, 18, 19, 20, 21, 22, 23, 24, 25, 26, 27, 28, 29, or 30 nucleotides. In some embodiments, the spacer sequence is at least 17 linked nucleotides in length. In some embodiments, the spacer sequence is at least 18 linked nucleotides in length. In some embodiments, the spacer region is at least 20 linked nucleotides in length. In some embodiments, the spacer sequence is at least 80%, at least 85%, at least 90%, at least 95% or 100% complementary to a target sequence of the target nucleic acid. In some embodiments, the spacer sequence is 100% complementary to the target sequence of the target nucleic acid. In some embodiments, the spacer sequence comprises at least 15 contiguous nucleotides that are complementary to the target nucleic acid. It is understood that the sequence of a spacer sequence need not be 100% complementary to that of a target sequence of a target nucleic acid to hybridize or hybridize specifically to the target sequence. The spacer sequence may comprise at least one nucleotide that is not complementary to the corresponding nucleotide of the target sequence. In some embodiments the spacer sequence is less than 100% complementary to the target sequence, but still can bind to the target sequence. In some embodiments the spacer sequence is 90%, 91%, 92%, 93%, 94%, 95%, 96%, 97%, 98%, 99%, or 100% complementary to the target sequence.

In some embodiments, a guide nucleic acid, a spacer region thereof or a spacer sequence thereof, comprises at least 10, 11, 12, 13, 14, 15, 16, 17, 18, 19, 20, 21, 22, 23, 24, 25, 26, 27, 28, 29, or 30 contiguous nucleotides that are complementary to a eukaryotic sequence. Such a eukaryotic sequence is a sequence of nucleotides that is present in a host eukaryotic cell. Such a sequence of nucleotides is distinguished from nucleotide sequences present in other host cells, such as prokaryotic cells, or viruses. By way of non-limiting example, said sequences present in a eukaryotic cell can be located in a gene, an exon, an intron, or a non-coding (e.g., promoter or enhancer) region. In some embodiments, a linker is present between the spacer and repeat sequences.

In some instances, a guide nucleic acid comprises a spacer length that confers nickase activity to a Cas enzyme that otherwise comprises nuclease activity when used with guide nucleic acids having a different spacer length. In some instances, the Cas enzyme is a Type V Cas enzyme. In some instances, the Cas enzyme is a CasPhi enzyme. In some cases, the Cas enzyme is CasPhi.12 (SEQ ID NO: 12). In some cases, the spacer length that confers nickase activity is 14-16 nucleotides. In some cases, the spacer length is 15 nucleotides. A non-limiting example of such systems is provided in Example 5.

### Nucleic Acid Linkers

In some embodiments, a guide nucleic acid for use with compositions, systems, and methods described herein comprises one or more linkers, or a nucleic acid encoding one or more linkers. In some embodiments, the guide nucleic acid comprises at least one, at least two, at least three, at least four, at least five, at least six, at least seven, at least eight, at least nine, or at least ten linkers. In some embodiments, the guide nucleic acid comprises one, two, three, four, five, six, seven, eight, nine, or ten linkers. In some embodiments, the guide nucleic acid comprises two or more linkers. In some embodiments, at least two or more linkers are the same. In some embodiments, at least two or more linkers are not same.

In some embodiments, a linker comprises one to ten, one to seven, one to five, one to three, two to ten, two to eight, two to six, two to four, three to ten, three to seven, three to five, four to ten, four to eight, four to six, five to ten, five to seven, six to ten, six to eight, seven to ten, or eight to ten linked nucleotides. In some embodiments, the linker comprises one, two, three, four, five, six, seven, eight, nine, or ten linked nucleotides. In some embodiments, a linker comprises a nucleotide sequence of 5′-GAAA-3′ (SEQ ID NO: 41).

In some embodiments, a guide nucleic acid comprises one or more linkers connecting one or more repeat sequences. In some embodiments, the guide nucleic acid comprises one or more linkers connecting one or more repeat sequences and one or more spacer sequences. In some embodiments, the guide nucleic acid comprises at least two repeat sequences connected by a linker.

### Repeat Sequences

Guide nucleic acids described herein may comprise one or more repeat sequences. In some embodiments, a repeat sequence comprises a nucleotide sequence that is not complementary to a target sequence of a target nucleic acid. In some embodiments, a repeat sequence comprises a nucleotide sequence that may interact with an effector protein. In some embodiments, a repeat sequence includes a nucleotide sequence that is capable of forming a guide nucleic acid-effector protein complex (e.g., a RNP complex). In some embodiments, the repeat sequence may also be referred to as a “protein-binding segment.”

In some embodiments, a repeat sequence is adjacent to a spacer sequence. In some embodiments, a repeat sequence is followed by a spacer sequence in the 5′ to 3′ direction. In some embodiments, a repeat sequence is adjacent to an intermediary sequence. In some embodiments, a repeat sequence is 3′ to an intermediary sequence. In some embodiments, an intermediary sequence is followed by a repeat sequence, which is followed by a spacer sequence in the 5′ to 3′ direction. In some embodiments, a repeat sequence is linked to a spacer sequence and/or an intermediary sequence. In some embodiments, a guide nucleic acid comprises a repeat sequence linked to a spacer sequence, which may be a direct link or by any suitable linker, examples of which are described herein.

In some embodiments, the spacer sequence(s) and the repeat sequence(s) of the guide nucleic acid are present within the same polynucleotide molecule. In some embodiments, a spacer sequence is adjacent to a repeat sequence. In some embodiments, a spacer sequence follows a repeat sequence in a 5′ to 3′ direction. In some embodiments, a spacer sequence precedes a repeat sequence in a 5′ to 3′ direction. In some embodiments, the spacer(s) and repeat sequence(s) are linked directly to one another. In some embodiments, a linker is present between the spacer(s) and repeat sequence(s). Linkers may be any suitable linker. In some embodiments, the spacer sequence(s) and the repeat sequence(s) of the guide nucleic acid are present in separate polynucleotide molecules, which are joined to one another by base pairing interactions.

In some embodiments, the repeat sequence comprises two sequences that are complementary to each other and hybridize to form a double stranded RNA duplex (dsRNA duplex). In some embodiments, the two sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired, and therefore the duplex forming sequence may include a bulge. In some embodiments, the repeat sequence comprises a hairpin or stem-loop structure, optionally at the 5′ portion of the repeat sequence. In some embodiments, a strand of the stem portion comprises a sequence and the other strand of the stem portion comprises a sequence that is, at least partially, complementary. In some embodiments, such sequences may have 65% to 100% complementarity (e.g., 65%, 70%, 75%, 80%, 85%, 90%, 95%, or 100% complementarity). In some embodiments, a guide nucleic acid comprises nucleotide sequence that when involved in hybridization events may hybridize over one or more segments such that intervening or adjacent segments are not involved in the hybridization event (e.g., a bulge, a loop structure or hairpin structure, etc.).

Exemplary repeat sequences are provided in Table 1.

### Intermediary Sequence

Guide nucleic acids described herein may comprise one or more intermediary sequences. In general, an intermediary sequence used in the present disclosure is not transactivated or transactivating. An intermediary sequence may also be referred to as an intermediary RNA, although it may comprise deoxyribonucleotides instead of or in addition to ribonucleotides, and/or modified bases. In general, the intermediary sequence non-covalently binds to an effector protein. In some embodiments, the intermediary sequence forms a secondary structure, for example in a cell, and an effector protein binds the secondary structure.

In some embodiments, a length of the intermediary sequence is at least 30, 50, 70, 90, 110, 130, 150, 170, 190, or 210 linked nucleotides. In some embodiments, a length of the intermediary sequence is not greater than 30, 50, 70, 90, 110, 130, 150, 170, 190, or 210 linked nucleotides. In some embodiments, the length of the intermediary sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

An intermediary sequence may also comprise or form a secondary structure (e.g., one or more hairpin loops) that facilitates the binding of an effector protein to a guide nucleic acid and/or modification activity of an effector protein on a target nucleic acid (e.g., a hairpin region). An intermediary sequence may comprise from 5′ to 3′, a 5′ region, a hairpin region, and a 3′ region. In some embodiments, the 5′ region may hybridize to the 3′ region. In some embodiments, the 5′ region of the intermediary sequence does not hybridize to the 3′ region.

In some embodiments, the hairpin region may comprise a first sequence, a second sequence that is reverse complementary to the first sequence, and a stem-loop linking the first sequence and the second sequence. In some embodiments, an intermediary sequence comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, an intermediary sequence comprises a pseudoknot (e.g., a secondary structure comprising a stem at least partially hybridized to a second stem or half-stem secondary structure). An effector protein may interact with an intermediary sequence comprising a single stem region or multiple stem regions. In some embodiments, the nucleotide sequences of the multiple stem regions are identical to one another. In some embodiments, the nucleotide sequences of at least one of the multiple stem regions is not identical to those of the others. In some embodiments, an intermediary sequence comprises 1, 2, 3, 4, 5 or more stem regions.

### Handle Sequence

In some embodiments, compositions, systems and methods described herein comprise the nucleic acid, wherein the nucleic acid comprises a handle sequence. In some embodiments, the handle sequence comprises an intermediary sequence. In some embodiments, the intermediary sequence is at the 3′-end of the handle sequence. In some embodiments, the intermediary sequence is at the 5′-end of the handle sequence. In some embodiments, the handle sequence further comprises one or more of linkers and repeat sequences. In some embodiments, the linker comprises a sequence of 5′-GAAA-3.′ In some embodiments, the intermediary sequence is 5′ to the repeat sequence. In some embodiments, the intermediary sequence is 5′ to the linker. In some embodiments, the intermediary sequence is 3′ to the repeat sequence. In some embodiments, the intermediary sequence is 3′ to the linker. In some embodiments, the repeat sequence is 3′ to the linker. In some embodiments, the repeat sequence is 5′ to the linker.

In some embodiments, a sgRNA may include a handle sequence having a hairpin region, as well as a linker and a repeat sequence. The sgRNA having a handle sequence can have a hairpin region positioned 3′ of the linker and/or repeat sequence. The sgRNA having a handle sequence can have a hairpin region positioned 5′ of the linker and/or repeat sequence. The hairpin region may include a first sequence, a second sequence that is reverse complementary to the first sequence, and a stem-loop linking the first sequence and the second sequence.

In some embodiments, an effector protein may recognize a secondary structure of a handle sequence. In some embodiments, at least a portion of the handle sequence interacts with an effector protein described herein. Accordingly, in some embodiments, at least a portion of the intermediary sequence interacts with the effector protein described herein. In some embodiments, both, at least a portion of the intermediary sequence and at least a portion of the repeat sequence, interacts with the effector protein. In general, the handle sequence is capable of interacting (e.g., non-covalent binding) with any one of the effector proteins described herein.

In some embodiments, the handle sequence of a sgRNA comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the sgRNA comprises a pseudoknot (e.g., a secondary structure comprising a stem at least partially hybridized to a second stem or half-stem secondary structure). An effector protein may recognize a sgRNA comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the sgRNA comprises at least 2, at least 3, at least 4, or at least 5 stem regions.

A handle sequence may include deoxyribonucleosides, ribonucleosides, chemically modified nucleosides, or any combination thereof. In some embodiments, a length of the handle sequence is at least 30, 50, 70, 90, 110, 130, 150, 170, 190, or 210 linked nucleotides. In some embodiments, a length of the handle sequence is not greater than 30, 50, 70, 90, 110, 130, 150, 170, 190, or 210 linked nucleotides. In some embodiments, the length of the handle sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

In some embodiments, the length of a handle sequence in a sgRNA is not greater than 50, 56, 66, 67, 68, 69, 70, 71, 72, 73, 95, or 105 linked nucleotides. In some embodiments, the length of a handle sequence in a sgRNA is about 30 to about 120 linked nucleotides. In some embodiments, the length of a handle sequence in a sgRNA is about 50 to about 105, about 50 to about 95, about 50 to about 73, about 50 to about 71, about 50 to about 70, or about 50 to about 69 linked nucleotides. In some embodiments, the length of a handle sequence in a sgRNA is 56 to 105 linked nucleotides, from 56 to 105 linked nucleotides, 66 to 105 linked nucleotides, 67 to 105 linked nucleotides, 68 to 105 linked nucleotides, 69 to 105 linked nucleotides, 70 to 105 linked nucleotides, 71 to 105 linked nucleotides, 72 to 105 linked nucleotides, 73 to 105 linked nucleotides, or 95 to 105 linked nucleotides. In some embodiments, the length of a handle sequence in a sgRNA is 40 to 70 nucleotides. In some embodiments, the length of a handle sequence in a sgRNA is 50, 56, 66, 67, 68, 69, 70, 71, 72, 73, 95, or 105 linked nucleotides.

### Single Nucleic Acid Systems

In some embodiments, compositions, systems and methods described herein comprise a single nucleic acid system comprising a guide nucleic acid or a nucleotide sequence encoding the guide nucleic acid, and one or more effector proteins or a nucleotide sequence encoding the one or more effector proteins. In some embodiments, a first region (FR) of the guide nucleic acid non-covalently interacts with the one or more polypeptides described herein. In some embodiments, a second region (SR) of the guide nucleic acid hybridizes with a target sequence of the target nucleic acid. In the single nucleic acid system having a complex of the guide nucleic acid and the effector protein, the effector protein is not transactivated by the guide nucleic acid. In other words, activity of effector protein does not require binding to a second non-target nucleic acid molecule. An exemplary guide nucleic acid for a single nucleic acid system is a crRNA or a sgRNA.

crRNA

In some embodiments, guide nucleic acid comprises a crRNA comprising a spacer sequence(s) and a repeat sequence(s) present within the same polynucleotide molecule. In some embodiments, the spacer sequence is adjacent to the repeat sequence. In some embodiments, the spacer sequence follows the repeat sequence in a 5′ to 3′ direction. In some embodiments, the spacer sequence precedes the repeat sequence in a 5′ to 3′ direction. In some embodiments, the spacer(s) and repeat sequence(s) are linked directly to one another. In some embodiments, a linker is present between the spacer(s) and repeat sequence(s). Linkers may be any suitable linker.

In some embodiments, a crRNA is useful as a single nucleic acid system for compositions, methods, and systems described herein or as part of a single nucleic acid system for compositions, methods, and systems described herein. In some embodiments, a crRNA is useful as part of a single nucleic acid system for compositions, methods, and systems described herein. In such embodiments, a single nucleic acid system comprises a guide nucleic acid comprising a crRNA wherein, a repeat sequence of a crRNA is capable of connecting a crRNA to an effector protein. In some embodiments, a single nucleic acid system comprises a guide nucleic acid comprising a crRNA linked to another nucleotide sequence that is capable of being non-covalently bond by an effector protein. In such embodiments, a repeat sequence of a crRNA can be linked to an intermediary RNA. In some embodiments, a single nucleic acid system comprises a guide nucleic acid comprising a crRNA and an intermediary RNA.

In some embodiments, a crRNA is sufficient to form complex with an effector protein (e.g., to form an RNP) through the repeat sequence and direct the effector protein to a target nucleic acid sequence through the spacer sequence. In some embodiments, the repeat sequence in the crRNA polynucleotide hybridizes with a tracr sequence present in a separate polynucleotide. In some embodiments, the hybridization with the tracr sequences permits formation of an RNP complex with an effector protein.

A crRNA may include deoxyribonucleosides, ribonucleosides, chemically modified nucleosides, or any combination thereof. In some embodiments, a crRNA comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In some embodiments, a crRNA comprises at least: 10, 15, 20, 25, 30, 35, 40, 45, 50, 55, 60 linked nucleotides. In some embodiments, the length of the crRNA is about 20 to about 120 linked nucleotides. In some embodiments, the length of a crRNA is about 20 to about 100, about 30 to about 100, about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a crRNA is about 20, about 25, about 30, about 35, about 40, about 45, about 50, about 55, about 60, about 65, about 70 or about 75 linked nucleotides.

sgRNA

In some embodiments, a guide nucleic acid comprises a single guide RNA (sgRNA). In some embodiments, the guide nucleic acid is a sgRNA. The combination of a spacer sequence (e.g., a nucleotide sequence that hybridizes to a target sequence in a target nucleic acid) with a handle sequence may be referred to herein as a single guide RNA (sgRNA), wherein the spacer sequence and the handle sequence are covalently linked. In some embodiments, the spacer sequence and handle sequence are linked by a phosphodiester bond. In some embodiments, the spacer sequence and handle sequence are linked by one or more linked nucleotides. In some embodiments, a guide nucleic acid may comprise a spacer sequence, a repeat sequence, or handle sequence, or a combination thereof. In some embodiments, the handle sequence may comprise a portion of, or all of, a repeat sequence. In general, a sgRNA comprises a first region (FR) and a second region (SR), wherein the FR comprises a handle sequence and the SR comprises a spacer sequence.

In some embodiments, the compositions comprising a guide RNA and an effector protein without a tracrRNA (e.g., a single nucleic acid system), wherein the guide RNA is a sgRNA. A sgRNA may include deoxyribonucleosides, ribonucleosides, chemically modified nucleosides, or any combination thereof. A sgRNA may also include a nucleotide sequence that forms a secondary structure (e.g., one or more hairpin loops) that facilitates the binding of an effector protein to the sgRNA and/or modification activity of an effector protein on a target nucleic acid (e.g., a hairpin region). Such a sequence can be contained within a handle sequence as described herein.

In some embodiments, a sgRNA comprises one or more of one or more of a handle sequence, an intermediary sequence, a crRNA, a repeat sequence, a spacer sequence, a linker, or combinations thereof. For example, a sgRNA comprises a handle sequence and a spacer sequence; an intermediary sequence and an crRNA; an intermediary sequence, a repeat sequence and a spacer sequence; and the like.

In some embodiments, a sgRNA comprises an intermediary sequence and an crRNA. In some embodiments, an intermediary sequence is 5′ to a crRNA in an sgRNA. In some embodiments, a sgRNA comprises a linked intermediary sequence and crRNA. In some embodiments, an intermediary sequence and a crRNA are linked in an sgRNA directly (e.g., covalently linked, such as through a phosphodiester bond) In some embodiments, an intermediary sequence and a crRNA are linked in an sgRNA by any suitable linker, examples of which are provided herein.

In some embodiments, a sgRNA comprises a handle sequence and a spacer sequence. In some embodiments, a handle sequence is 5′ to a spacer sequence in an sgRNA. In some embodiments, a sgRNA comprises a linked handle sequence and spacer sequence. In some embodiments, a handle sequence and a spacer sequence are linked in an sgRNA directly (e.g., covalently linked, such as through a phosphodiester bond) In some embodiments, a handle sequence and a spacer sequence are linked in an sgRNA by any suitable linker, examples of which are provided herein.

In some embodiments, a sgRNA comprises an intermediary sequence, a repeat sequence, and a spacer sequence. In some embodiments, an intermediary sequence is 5′ to a repeat sequence in an sgRNA. In some embodiments, a sgRNA comprises a linked intermediary sequence and repeat sequence. In some embodiments, an intermediary sequence and a repeat sequence are linked in an sgRNA directly (e.g., covalently linked, such as through a phosphodiester bond) In some embodiments, an intermediary sequence and a repeat sequence are linked in an sgRNA by any suitable linker, examples of which are provided herein. In some embodiments, a repeat sequence is 5′ to a spacer sequence in an sgRNA. In some embodiments, a sgRNA comprises a linked repeat sequence and spacer sequence. In some embodiments, a repeat sequence and a spacer sequence are linked in an sgRNA directly (e.g, covalently linked, such as through a phosphodiester bond) In some embodiments, a repeat sequence and a spacer sequence are linked in an sgRNA by any suitable linker, examples of which are provided herein.

An exemplary handle sequence in a sgRNA may comprise, from 5′ to 3′, a 5′ region, a hairpin region, and a 3′ region. In some embodiments, the 5′ region may hybridize to the 3′ region. In some embodiments, the 5′ region does not hybridize to the 3′ region. In some embodiments, the 3′ region is covalently linked to a spacer sequence (e.g., through a phosphodiester bond). In some embodiments, the 5′ region is covalently linked to a spacer sequence (e.g., through a phosphodiester bond).

### Dual Nucleic Acid Systems

In some embodiments, compositions, systems and methods described herein comprise a dual nucleic acid system comprising a crRNA or a nucleotide sequence encoding the crRNA, a tracrRNA or a nucleotide sequence encoding the tracrRNA, and one or more effector proteins or a nucleotide sequence encoding the one or more effector proteins, wherein the crRNA and the tracrRNA are separate, unlinked molecules, wherein a repeat hybridization region of the tracrRNA is capable of hybridizing with an equal length portion of the crRNA to form a tracrRNA-crRNA duplex, wherein the equal length portion of the crRNA does not include a spacer sequence of the crRNA, and wherein the spacer sequence is capable of hybridizing to a target sequence of the target nucleic acid. In the dual nucleic acid system having a complex of the guide nucleic acid, tracrRNA, and the effector protein, the effector protein is transactivated by the tracrRNA. In other words, in a dual nucleic acid system, activity of the effector protein requires binding to a tracrRNA molecule.

In some embodiments, a repeat hybridization sequence is at the 3′ end of a tracrRNA. In some embodiments, a repeat hybridization sequence may have a length of about 1, about 2, about 3, about 4, about 5, about 6, about 7, about 8, about 9, about 10, about 12, about 14, about 16, about 18, or about 20 linked nucleotides. In some embodiments, the length of the repeat hybridization sequence is 1 to 20 linked nucleotides.

In some embodiments, systems, compositions, and methods comprise a crRNA or a use thereof. In general, a crRNA comprises a first region (FR) and a second region (SR), wherein the FR of the crRNA comprises a repeat sequence, and the SR of the crRNA comprises a spacer sequence. In some embodiments, the repeat sequence and the spacer sequences are directly connected to each other (e.g., covalent bond (phosphodiester bond)). In some embodiments, the repeat sequence and the spacer sequence are connected by a linker.

In some embodiments, systems, compositions, and methods comprise a tracrRNA or a use thereof. In some embodiments, systems, compositions, and methods do not comprise a tracrRNA or a use thereof. A tracrRNA and/or tracrRNA-crRNA duplex may form a secondary structure that facilitates the binding of an effector protein to a tracrRNA or a tracrRNA-crRNA. In some embodiments, the secondary structure modifies activity of the effector protein on a target nucleic acid. In some embodiments, the secondary structure comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the secondary structure comprises a pseudoknot (e.g., a secondary structure comprising a stem at least partially hybridized to a second stem or half-stem secondary structure). An effector protein may recognize a secondary structure comprising multiple stem regions. In some embodiments, nucleotide sequences of the multiple stem regions are identical to one another. In some embodiments, the nucleotide sequences of at least one of the multiple stem regions is not identical to those of the others. In some embodiments, the secondary structure comprises at least two, at least three, at least four, or at least five stem regions. In some embodiments, the secondary structure comprises one or more loops. In some embodiments, the secondary structure comprises at least one, at least two, at least three, at least four, or at least five loops.

### Guide Nucleic Acids for Precision Editing

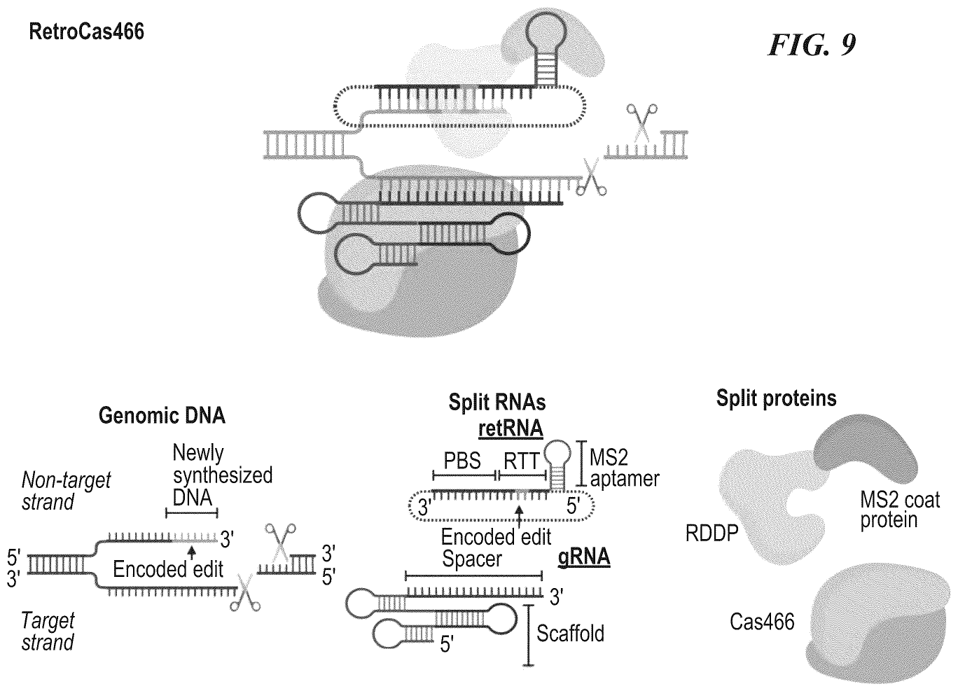

In some embodiments, the present disclosure provides guide nucleic acids for use in combination with the effector proteins, RDDPs, and fusion proteins thereof described herein for precision editing of a target nucleic acids sequence. In general, guide nucleic acids for use in precision editing comprise a spacer sequence, a repeat sequence, a primer binding sequence, and a template sequence. In some embodiments, guide nucleic acids for use in precision editing comprise one or more linkers between one or more components of the guide nucleic acids. In some embodiments, a spacer sequence, a repeat sequence, a primer binding sequence, and a template sequence are comprised in a single polynucleotide, referred to herein as an extended guide RNA (rtgRNA). See e.g., FIG. 3 and FIG. 4. In some embodiments, a spacer sequence, a repeat sequence, a primer binding sequence, and a template sequence are comprised in two polynucleotides—the spacer and repeat sequence comprised in a first polynucleotide and the primer binding sequence and the template sequence comprised in a second polynucleotide, referred to herein as a split RNA. See e.g., FIG. 5A, FIG. 5B, and FIG. 9.

### Template RNA

In some instances, compositions, systems and methods described herein comprise a template RNA, wherein the template RNA comprises a primer binding sequence and a template sequence. The template RNA may also be referred to as an extension of a guide RNA. The extension may comprise 1, 2, 3, 4, 5, 6, 7, 8, 9, 10, 11, 12, 13, 14, 15, 16, 17, 18, 19, or 20 nucleotides. The extension may comprise more than 10 nucleotides. In some embodiments the extension is 10-20 nucleotides. In some embodiments the extension is 20-30 nucleotides. In some embodiments the extension is 30-40 nucleotides. In some embodiments the extension is 40-50 nucleotides. In some embodiments the extension is 50-60 nucleotides. In some embodiments the extension is 70-80 nucleotides. In some embodiments the extension is 80-90 nucleotides. In some embodiments the extension is 90-100 nucleotides. In some embodiments the extension is 100-150 nucleotides. The extension may be processed during guide RNA formation. Template RNAs are also referred to herein as rttRNAs and retRNAs.

In some embodiments, the template RNA, the spacer sequence, and the repeat sequence are comprised in the same polynucleotide (e.g., an rtgRNA). In some embodiments, the spacer sequence and repeat sequence are comprised in a first polynucleotide and the template RNA is comprised in a second polynucleotide (e.g., a split RNA system).

In some instances, the primer binding sequence hybridizes to a primer sequence on the non-target strand of the target dsDNA molecule. In some instances, the primer binding sequence hybridizes to a primer sequence on the target strand of the target dsDNA molecule. In some embodiments, the spacer sequence is complementary to the target sequence on the target strand of the dsDNA molecule, and the primer binding sequence and/or the template sequence is complementary to a primer sequence on the non-target strand of the target dsDNA molecule. In some embodiments, the spacer sequence is complementary to the target sequence on the non-target strand of the dsDNA molecule, and the primer binding sequence and/or the template sequence is complementary to a primer sequence on the target strand of the target dsDNA molecule.

In some embodiments, the primer binding sequence is 4, 5, 6, 7, 8, 9, 10, 11, 12, 13, 14, 15, 16, 17, 18, 19, or 20 nucleotides long. In some embodiments the template sequence is at least 10, 11, 12, 13, 14, 15, 16, 17, 18, 19, 20, 21, 22, 23, 24, 25, 26, 27, 28, 29, 30, 31, 32, 33, 34 or 35 nucleotides long. In some embodiments, at least a portion of the PBS is complementary at least a portion of the target nucleic acid sequence that is 5′ of the nucleotide at position 13 relative to the PAM sequence. Such embodiments are particularly useful when used in a system comprising a CasM.265466 effector protein.

The template sequence may comprise one or more nucleotides having a different nucleobase than that of a nucleotide at the corresponding position in the target nucleic acid when a spacer sequence of the guide RNA and the target sequence are aligned for maximum identity. The one or more nucleotides may be contiguous. The one or more nucleotides may not be contiguous. The one or more nucleotides may each independently be selected from guanine, adenine, cytosine and thymine.

Extended Guide RNA (rtgRNA) Systems

In some embodiments, the present disclosure provides extended guide nucleic acids (rtgRNA) and an effector protein and RDDP, or fusion protein thereof described herein, or nucleic acids encoding the same, wherein the spacer sequence, repeat sequence, template sequence, and primer binding sequence are each comprised in a single polynucleotide.

In some embodiments, the rtgRNA comprises, from 5′ to 3′, a template sequence, a primer binding sequence, a repeat sequence, and a spacer sequence, optionally wherein a linker sequence is located between the primer binding sequence and the repeat sequence. See e.g., FIG. 3. In some embodiments, the rtgRNA comprises, from 5′ to 3′, a repeat sequence, a spacer sequence, a template sequence, and a primer binding sequence, optionally wherein a linker sequence is located between the template sequence and the spacer sequence. See e.g., FIG. 4.

Split gRNA Systems

In some embodiments, the present disclosure provides a split gRNA system, comprising a first polynucleotide comprising a spacer sequence and a repeat sequence (e.g., a gRNA) and a second polynucleotide comprising a primer binding sequence and a template sequence (e.g., an rttRNA), and an effector protein and RDDP, or fusion protein thereof described herein, or nucleic acids encoding the same.

In some embodiments, the first polynucleotide comprises a spacer sequence and a repeat sequence. In some embodiments, the first polynucleotide is a crRNA, as described above. In some embodiments, the first polynucleotide comprises a spacer sequence and a handle sequence (also referred to herein as a scaffold sequence). In some embodiments, the first polynucleotide is an sgRNA, as described above.

In some embodiments, the second polynucleotide comprises a primer binding sequence and a template sequence (e.g., an rttRNA). In some embodiments, the second polynucleotide further comprises an aptamer that is recognized by a biological tether protein linked to an RDDP described herein. In some embodiments, the aptamer is an MS2 aptamer (See Said et al (November 2009). “In vivo expression and purification of aptamer-tagged small RNA regulators”. Nucleic Acids Research. 37 (20): c133; and Johansson et al (1997). “RNA recognition by the MS2 phage coat protein”. Seminars in Virology. 8 (3): 176-185). In some embodiments, the second polynucleotide comprises, from 5′ to 3′, an aptamer sequence, a template sequence, and a primer binding sequence. See FIG. 5B. In some embodiments, the second polynucleotide comprises, from 5′ to 3′, template sequence, a primer binding sequence, and an aptamer sequence. See FIG. 5A. In FIG. 5A and FIG. 5B, an MS2 aptamer is shown on the 3′ and 5′ ends of the rttRNA, respectively. The MS2 aptamer is an exemplary sequence and an RNA sequence with the appropriate secondary structure may also be used instead of the aptamer.

In some embodiments, the second polynucleotide is circularized. Sec FIG. 9.

### Integrases

In some embodiments, guide nucleic acids comprise an integration sequence. In some embodiments, the retRNA comprises an integration sequence. In some embodiments, the RTT sequence of the retRNA comprises an integration sequence. In some instances, the integration sequence is linked to a primer binding sequence. The guide nucleic acid may interact with the effector protein and target the effector protein to the desired location in the cell genome. The effector protein may nick a strand of the cell genome and the RDDP may incorporate the integration sequence of the guide nucleic acid into the nicked site. This provides an integration site at the desired location of the cell genome. In some embodiments, compositions and systems further comprise a donor nucleic acid comprising a sequence that is complementary to the integration site, and an integration enzyme, wherein the integration enzyme incorporates the donor nucleic acid into the cell genome at the integration site by integration, recombination, or reverse transcription of the sequence that is complementary to the integration site.

The integration enzyme can be a recombinase that incorporates the genome or nucleic acid of interest into the cell genome at the integration site by recombination. The integration enzyme can be an RNA-directed DNA polymerase, which in some embodiments can be a reverse transcriptase that incorporates the genome or nucleic acid of interest into the cell genome at the integration site by reverse transcription. The integration enzyme can be a retrotransposase that incorporates the genome or nucleic acid of interest into the cell genome at the integration site by retrotransposition.

In some embodiments, the integration enzyme is selected from the group consisting of Hin, Gin, Tn3, β-six, CinH, ParA, γδ, TP901, φBT1, φRV1, φFC1, A118, U153, gp29, FLP, R, Lambda, HK101, HK022, and pSAM2, Cre, Dre, Vika, Bxbl, φC31, RDF, FLP, qBTI, R1, R2, R3, R4, R5, TP901-1, A118, φCl, MRII, TGI, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, L1, Tol2 Tcl, Tc3, Mariner (Himar 1), Mariner (mos 1), and Minos, and any mutants thereof. In some embodiments the integration enzyme is listed in WO2022087235, which is incorporated herein.

In some embodiments, the integration site can be selected from an attB site, an attP site, an attL site, an attR site, a lox71 site a Vox site, a Bxbl site, or a FRT site.

In some embodiments multiple genes are modified. In some embodiments the multiplexing is done by using multiple different integration sites with multiple different guide and effector proteins.

### Target Nucleic Acids and Samples

Described herein are compositions, systems and methods for modifying a target nucleic acid, wherein the target nucleic acid is a gene, a portion thereof, a transcript thereof. In some embodiments, the target nucleic acid is a reverse transcript (e.g., acDNA) of an mRNA transcribed from the gene, or an amplicon thereof. acid. In some embodiments, the target nucleic acid is a single stranded nucleic acid. Alternatively, or in combination, the target nucleic acid is a double stranded nucleic acid and is prepared into single stranded nucleic acids before or upon contacting the reagents. In some embodiments, the target nucleic acid is a double stranded nucleic acid. In some embodiments, the double stranded nucleic acid is DNA. The target nucleic acid may be an RNA. The target nucleic acids include but are not limited to mRNA, rRNA, tRNA, non-coding RNA, long non-coding RNA, and microRNA (miRNA). In some embodiments, the target nucleic acid is complementary DNA (cDNA) synthesized from a single-stranded RNA template in a reaction catalyzed by an RNA-directed DNA polymerase, which in some embodiments is a reverse transcriptase. In some embodiments, the target nucleic acid is single-stranded RNA (ssRNA) or mRNA. In some embodiments, the target nucleic acid is from a virus, a parasite, or a bacterium described herein. As another non-limiting example, the target nucleic acid may be responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides).

The nucleic acid may be single-stranded or double stranded, linear or circular, segmented or non-segmented. The nucleic acid may comprise DNA, RNA, or a combination thereof. Non-limiting examples of viruses or viral particles that can deliver a viral vector include retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. A viral vector delivered by such viruses or viral particles may be referred to by the type of virus to deliver the viral vector (e.g., an AAV viral vector is a viral vector that is to be delivered by an adeno-associated virus). A viral vector referred to by the type of virus to be delivered by the viral vector can contain viral elements (e.g., nucleotide sequences) necessary for packaging of the viral vector into the virus or viral particle, replicating the virus, or other desired viral activities. A virus containing a viral vector may be replication competent, replication deficient or replication defective.

In certain embodiments, the target nucleic acid is a double stranded nucleic acid comprising a target strand and a non-target strand, wherein the target strand comprises a target sequence. In some embodiments, where a target strand comprises a target sequence, at least a portion of the guide nucleic acid is complementary to the target sequence on the target strand. In some embodiments, where the target nucleic acid is a double stranded nucleic acid comprising a target strand and a non-target strand, and wherein the target strand comprises a target sequence, at least a portion of the guide nucleic acid is complementary to the target sequence on the target strand. In some embodiments, a target nucleic acid comprises a PAM as described herein that is located on the non-target strand. Such a PAM described herein, in some embodiments, is adjacent (e.g., within 1, 2, 3, 4, 5, 10, 20, 25 nucleotides) to the 5′ or 3′ end of the target sequence on the non-target strand of the double stranded DNA molecule. In certain embodiments, such a PAM described herein is directly adjacent to the 5′ or 3′ end of a target sequence on the non-target strand of the double stranded DNA molecule.

In some embodiments, an effector protein described herein, or a multimeric complex thereof, recognizes a PAM on a target nucleic acid. In some embodiments, multiple effector proteins of the multimeric complex recognize a PAM on a target nucleic acid. In some embodiments, only one effector protein of the multimeric complex recognizes a PAM on a target nucleic acid. In some embodiments, the PAM is 3′ to the spacer region of the crRNA. In some embodiments, the PAM is directly or adjacent (e.g., within 1, 2, 3, 4, 5, 10, 20, 25 nucleotides) 3′ or 5′ to the spacer region of the crRNA. In some embodiments, the PAM comprises a PAM sequence set forth in TABLE 1.

An effector protein or fusion protein of the present disclosure may cleave or nick a target nucleic acid within or near a protospacer adjacent motif (PAM) sequence of the target nucleic acid. In some embodiments, cleavage occurs within 1, 2, 3, 4, 5, 6, 7, 8, 9, 10, 15, 20, or 25 nucleotides of a 5′ or 3′ terminus of a PAM sequence. A target nucleic acid may comprise a PAM sequence adjacent to a sequence that is complementary to a guide nucleic acid spacer region.

In some embodiments, the target nucleic acid comprises 5 to 100, 5 to 90, 5 to 80, 5 to 70, 5 to 60, 5 to 50, 5 to 40, 5 to 30, 5 to 25, 5 to 20, 5 to 15, or 5 to 10 linked nucleotides. In some embodiments, the target nucleic acid comprises 10 to 90, 20 to 80, 30 to 70, or 40 to 60 linked nucleotides. In some embodiments, the target nucleic acid comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 linked nucleotides. In some embodiments, the target nucleic acid comprises at least 10, at least 20, at least 30, at least 40, at least 50, at least 60, at least 70, at least 80, at least 90, or at least 100 linked nucleotides.

In some embodiments, the guide nucleic acid can bind to a target sequence, wherein the target sequence is eukaryotic. The guide nucleic acid may bind to a target nucleic acid, such as DNA or RNA, from a cancer gene or gene associated with a genetic disorder, or an amplicon thereof, as described herein. In some embodiments, the guide nucleic acid comprises a region that is complementary to an equal length portion of a gene selected from TABLE 5. The guide nucleic acid may bind to a target nucleic acid such as a nucleic acid from a bacterium, a virus, a parasite, a protozoon, a fungus or other agents responsible for a disease, or an amplicon thereof. The target nucleic acid may comprise a mutation, such as a single nucleotide polymorphism (SNP). A mutation may confer for example, resistance to a treatment, such as antibiotic treatment.

In some embodiments, the target nucleic acid comprises a portion or a specific region of a nucleic acid from a genomic locus, any DNA amplicon of, a reverse transcribed mRNA, or a cDNA from a gene described herein. In some embodiments, the target nucleic acid is an amplicon of at least a portion of a gene. Non-limiting examples of genes are set forth in TABLE 5. Nucleic acid sequences of target nucleic acids and/or corresponding genes are readily available in public databases as known and used in the art. In some embodiments, the target nucleic acid is selected from TABLE 5.

In some embodiments, the target nucleic acid comprises a target locus. In certain embodiments, the target nucleic acid comprises more than one target loci. In some embodiments, the target nucleic acid comprises two target loci. Accordingly, in some embodiments, the target nucleic acid can comprise one or more target sequences.

In some embodiments, the one or more target sequence is within any one of the genes set forth in TABLE 5. In some embodiments, the target sequence is within an exon of any one of the genes set forth in TABLE 5. In some embodiments, then target sequence covers the junction of two exons. In some embodiments, the target sequence is located within about 1 to about 300 nucleotides, about 10 to about 250, about 20 to about 200, about 30 to about 150, about 40 to about 100, or about 50 nucleotides of the 5′ untranslated region (UTR). In some embodiments, the target sequence is located within about 1 to about 300 nucleotides, about 10 to about 250, about 20 to about 200, about 30 to about 150, about 40 to about 100, or about 50 nucleotides of the 3′ UTR.

In some embodiments, the target sequence is at least partially within a targeted exon within any one of the genes set forth in TABLE 5. A targeted exon can mean any portion within, contiguous with, or adjacent to a specified exon of interest can be targeted by the compositions, systems, and methods described herein. In some embodiments, one or more of the exons are targeted. In some embodiments, one or more of exons of any one the genes set forth in TABLE 5 are targeted.

In some embodiments, a target sequence that a guide nucleic acid binds to is at least partially within a targeted exon within any one of the genes set forth in TABLE 5, and wherein at least a portion of the target nucleic acid is within a sequence about 1 to about 300 nucleotides adjacent to: the start of a targeted exon, the end of a targeted exon, or both. In some embodiments, at least a portion of the target sequence that a guide nucleic acid binds to can comprise a sequence about 1 to about 300 nucleotides, about 10 to about 250, about 20 to about 200, about 30 to about 150, about 40 to about 100, or about 50 nucleotides adjacent to: the start of a targeted exon, the end of a targeted exon, or both.

In some embodiments, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by an effector protein system.

In some embodiments, the target nucleic acid is in a cell. In some embodiments, the cell is a single-cell eukaryotic organism; a plant cell an algal cell; a fungal cell; an animal cell; a cell an invertebrate animal; a cell a vertebrate animal such as fish, amphibian, reptile, bird, and mammal; or a cell a mammal such as a human, a non-human primate, an ungulate, a feline, a bovine, an ovine, and a caprine. In exemplary embodiments, the cell is a eukaryotic cell. In exemplary embodiments, the cell is a mammalian cell, a human cell, or a plant cell. In some embodiments, the cell is a human cell.

### Mutations

In some embodiments, target nucleic acids comprise a mutation. In some embodiments, a composition, system or method described herein can be used to modify a target nucleic acid comprising a mutation such that the mutation is modified to be a wild-type nucleotide or nucleotide sequence. In some embodiments, a composition, system or method described herein can be used to detect a target nucleic acid comprising a mutation. In some embodiments, a sequence comprising a mutation may be modified to a wild-type sequence with a composition, system or method described herein.

A mutation may be in an open reading frame of a target nucleic acid. A mutation may result in the insertion of at least one amino acid in a protein encoded by the target nucleic acid. A mutation may result in the deletion of at least one amino acid in a protein encoded by the target nucleic acid. A mutation may result in the substitution of at least one amino acid in a protein encoded by the target nucleic acid. A mutation that results in the deletion, insertion, or substitution of one or more amino acids of a protein encoded by the target nucleic acid may result in misfolding of a protein encoded by the target nucleic acid. A mutation may result in a premature stop codon, thereby resulting in a truncation of the encoded protein.

In some embodiments, a mutation comprises a point mutation or single nucleotide polymorphism (SNP), a chromosomal mutation, a copy number mutation, or any combination thereof. A point mutation optionally comprises a substitution, insertion, or deletion.

In some embodiments, target nucleic acids comprise a mutation, wherein the mutation is a SNP. The single nucleotide mutation or SNP may be associated with a phenotype of the sample or a phenotype of the organism from which the sample was taken. The SNP, in some embodiments, is associated with altered phenotype from wild type phenotype. In some embodiments, a single nucleotide mutation, SNP, or deletion described herein is associated with a disease, such as a genetic disease. The SNP may be a synonymous substitution or a nonsynonymous substitution. The nonsynonymous substitution may be a missense substitution or a nonsense point mutation. The synonymous substitution may be a silent substitution. The mutation may be a deletion of one or more nucleotides. Often, the single nucleotide mutation, SNP, or deletion is associated with a disease such as cancer or a genetic disorder. The mutation, such as a single nucleotide mutation, a SNP, or a deletion, may be encoded in the sequence of a target nucleic acid from the germline of an organism or may be encoded in a target nucleic acid from a diseased cell, such as a cancer cell.

In some embodiments, the target nucleic acid comprises a mutation associated with a disease. In some examples, a mutation associated with a disease refers to a mutation whose presence in a subject indicates that the subject is susceptible to or suffers from, a disease, disorder, condition, or syndrome. In some examples, a mutation associated with a disease refers to a mutation which causes, contributes to the development of, or indicates the existence of the disease, disorder, condition, or syndrome. A mutation associated with a disease may also refer to any mutation which generates transcription or translation products at an abnormal level, or in an abnormal form, in cells affected by a disease relative to a control without the disease. In some examples, a mutation associated with a disease refers to a mutation whose presence in a subject indicates that the subject is susceptible to, or suffers from, a disease, disorder, or pathological state. In some embodiments, a mutation associated with a disease, comprises the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

### Methods of Treating a Disorder

Compositions, systems and methods disclosed herein may be useful for treating a disease in a subject by modifying a target nucleic acid associated with a gene or expression of a gene related to the disease. In some embodiments, methods comprise administering a composition or cell described herein to a subject. By way of non-limiting example, the disease may be a cancer, an ophthalmological disorder, a neurological disorder, a neurodegenerative disease, a blood disorder, or a metabolic disorder, or a combination thereof. The disease may be an inherited disorder, also referred to as a genetic disorder. The disease may be the result of an infection or associated with an infection.

In some embodiments, the present disclosure provides a method of treating a disease in a subject comprising administering an RDDP and an effector protein described herein, or fusion protein comprising the same, and an rtgRNA described herein to the subject. In some embodiments, the present disclosure provides a method of treating a disease in a subject comprising administering one or more polynucleotides encoding one or more components of a system described herein, or one or more vectors comprising the same, e.g., a polynucleotide encoding an RDDP described herein (or a fusion protein comprising the same) or vector comprising the same, a polynucleotide encoding an effector protein described herein (or a fusion protein comprising the same) or a vector comprising the same, and/or a polynucleotide encoding an rtgRNA or a vector comprising the same.

The compositions and methods described herein may be used to treat, prevent, or inhibit a disease or syndrome in a subject. In some embodiments, the disease is a liver disease, a lung disease, an eye disease, or a muscle disease. Exemplary diseases and syndromes include, but are not limited to, the diseases and syndromes listed in TABLE 6.

In some embodiments, compositions and methods modify at least one gene associated with the disease or the expression thereof. In some embodiments, the disease is Alzheimer's disease, and the gene is selected from APP, BACE-1, PSD95, MAPT, PSEN1, PSEN2, APOE, TARDBP, and APOE&4. In some embodiments, the disease is dementia and the gene is TARDBP. In some embodiments, the disease is Pick's disease and the gene is TARDBP. In some embodiments, the disease is Parkinson's disease, and the gene is selected from SNCA, GDNF, and LRRK2. In some embodiments, the disease comprises Centronuclear myopathy, and the gene is DNM2. In some embodiments, the disease is Huntington's disease, and the gene is HTT. In some embodiments, the disease is Alpha-1 antitrypsin deficiency (AATD) and the gene is SERPINA1. In some embodiments, the disease is amyotrophic lateral sclerosis (ALS), and the gene is selected from SOD1, FUS, C9ORF72, ATXN2, TARDBP, and CHCHD10. In some embodiments, the disease comprises Alexander Disease and the gene is GFAP. In some embodiments, the disease comprises anaplastic large cell lymphoma and the gene is CD30. In some embodiments, the disease comprises Angelman Syndrome and the gene is UBE3A. In some embodiments, the disease comprises calcific aortic stenosis and the gene is Apo (a). In some embodiments, the disease comprises CD3Z-associated primary T-cell immunodeficiency and the gene is CD3Z or CD247. In some embodiments, the disease comprises CD18 deficiency, and the gene is ITGB2. In some embodiments, the disease comprises CD40L deficiency, and the gene is CD40L. In some embodiments, the disease comprises CNS trauma and the gene is VEGF. In some embodiments, the disease comprises coronary heart disease and the gene is selected from FGA, FGB, and FGG. In some embodiments, the disease comprises MECP2 Duplication syndrome and Rett syndrome and the gene is MECP2. In some embodiments, the disease comprises a bleeding disorder (coagulation) and the gene is FXI. In some embodiments, the disease comprises fragile X syndrome and the gene is FMR1. In some embodiments, the disease comprises Fuchs corneal dystrophy and the gene is selected from ZEB1, SLC4A11, and LOXHD1. In some embodiments, the disease comprises GM2-Gangliosidoses (e.g., Tay Sachs Disease, Sandhoff disease) and the gene is selected from HEXA and HEXB. In some embodiments, the disease comprises Hearing loss disorders and the gene is DFNA36. In some embodiments, the disease is Pompe disease, including infantile onset Pompe disease (IOPD) and late onset Pompe disease (LOPD) and the gene is GAA. In some embodiments, the disease is Retinitis pigmentosa and the gene is selected from PDE6B, RHO, RP1, RP2, RPGR, PRPH2, IMPDH1, PRPF31, CRB1, PRPF8, TULP1, CA4, HPRPF3, ABCA4, EYS, CERKL, FSCN2, TOPORS, SNRNP200, PRCD, NR2E3, MERTK, USH2A, PROMI, KLHL7, CNGB1, TTC8, ARL6, DHDDS, BESTI, LRAT, SPARA7, CRX, CLRN1, RPE65, and WDR19. In some embodiments, the disease comprises Leber Congenital Amaurosis Type 10 and the gene is CEP290. In some embodiments, the disease is cardiovascular disease and/or lipodystrophies and the gene is selected from ABCG5, ABCG8, AGT, ANGPTL3, APOCIII, APOE, APOA1, APOL1, ARH, CDKN2B, CFB, CXCL12, FXI, FXII, GATA-4, MIA3, MKL2, MTHFD1L, MYH7, NKX2-5, NOTCH1, PKK, PCSK9, PSRC1, SMAD3, and TTR. In some embodiments, the disease comprises acromegaly and the gene is GHR. In some embodiments, the disease comprises acute myeloid leukemia and the gene is CD22. In some embodiments, the disease is diabetes, and the gene is selected from GCGR and ANGPTL7. In some embodiments, the disease is NAFLD/NASH, and the gene is selected from DGAT2 and PNPLA3. In some embodiments, the disease is cancer, and the gene is selected from STAT3, YAP1, FOXP3, AR (Prostate cancer), and IRF4 (multiple myeloma). In some embodiments, the disease is cystic fibrosis, and the gene is CFTR. In some embodiments, the disease is Duchenne muscular dystrophy, and the gene is DMD. In some embodiments, the disease is facioscapulohumeral muscular dystrophy (FSHD) and the gene is DUX4. In some embodiments, the disease comprises angioedema and the gene is PKK. In some embodiments, the disease comprises thalassemia and the gene is TMPRSS6. In some embodiments, the disease comprises achondroplasia and the gene is FGFR3. In some embodiments, the disease comprises Cri du chat syndrome and the gene is selected from CTNND2. In some embodiments, the disease comprises sickle cell anemia and the gene is Beta globin gene. In some embodiments, the disease comprises Alagille Syndrome and the gene is selected from JAG1 and NOTCH2. In some embodiments, the disease comprises Charcot Maric Tooth disease and the gene is selected from PMP22 and MFN2. In some embodiments, the disease comprises Crouzon syndrome and the gene is selected from FGFR2, FGFR3, and FGFR3. In some embodiments, the disease comprises Dravet Syndrome and the gene is selected from SCNIA and SCN2A. In some embodiments, the disease comprises Emery-Dreifuss syndrome, and the gene is selected from EMD, LMNA, SYNE1, SYNE2, FHL1, and TMEM43. In some embodiments, the disease comprises Factor V Leiden thrombophilia and the gene is F5. In some embodiments, the disease comprises Fanconi anemia, and the gene is selected from FANCA, FANCB, FANCC, FANCD1, FANCD2, FANCE, FANCF, FANCG, FANCI, FANCJ, FANCL, FANCM, FANCN, FANCP, FANCS, RAD51C, and XPF. In some embodiments, the disease comprises Familial Creutzfeld-Jakob disease and the gene is PRNP. In some embodiments, the disease comprises Familial Mediterranean Fever and the gene is MEFV. In some embodiments, the disease comprises Friedreich's ataxia and the gene is FXN. In some embodiments, the disease comprises Gaucher disease, and the gene is GBA. In some embodiments, the disease comprises human papilloma virus (HPV) infection and the gene is HPV E7. In some embodiments, the disease comprises hemochromatosis and the gene is HFE, optionally comprising a C282Y mutation. In some embodiments, the disease comprises Hemophilia A and the gene is FVIII. In some embodiments, the disease comprises histiocytosis and the gene is CD1. In some embodiments, the disease comprises immunodeficiency 17 and the gene is CD3D. In some embodiments, the disease comprises immunodeficiency 13 and the gene is CD4. In some embodiments, the disease comprises Common Variable Immunodeficiency and the gene is selected from CD19 and CD81. In some embodiments, the disease comprises Joubert syndrome, and the gene is selected from INPP5E, TMEM216, AHI1, NPHP1, CEP290, TMEM67, RPGRIP1L, ARL13B, CC2D2A, OFD1, TMEM138, TCTN3, ZNF423, and AMRC9. In some embodiments, the disease comprises leukocyte adhesion deficiency and the gene is CD18. In some embodiments, the disease comprises Li-Fraumeni syndrome and the gene is TP53. In some embodiments, the disease comprises lymphoproliferative syndrome and the gene is CD27. In some embodiments, the disease comprises Lynch syndrome and the gene is selected from MSH2, MLH1, MSH6, PMS2, PMS1, TGFBR2, and MLH3. In some embodiments, the disease comprises mantle cell lymphoma and the gene is CD5. In some embodiments, the disease comprises Marfan syndrome and the gene is FBN1. In some embodiments, the disease comprises mastocytosis and the gene is CD2. In some embodiments, the disease comprises methylmalonic acidemia and the gene is selected from MMAA, MMAB, and MUT. In some embodiments, the disease is mycosis fungoides and the gene is CD7. In some embodiments, the disease is myotonic dystrophy, and the gene is selected from CNBP and DMPK. In some embodiments, the disease comprises neurofibromatosis and the gene is selected from NF1, and NF2. In some embodiments, the disease comprises osteogenesis imperfecta and the gene is selected from COL1A1, COL1A2, and IFITM5. In some embodiments, the disease is non-small cell lung cancer, and the gene is selected from KRAS, EGFR, ALK, METex 14, BRAF V600E, ROS1, RET, and NTRK. In some embodiments, the disease comprises Peutz-Jeghers syndrome and the gene is STK11. In some embodiments, the disease comprises polycystic kidney disease and the gene is selected from PKD1 and PKD2. In some embodiments, the disease comprises Severe Combined Immune Deficiency and the gene is selected from IL7R, RAGI, JAK3. In some embodiments, the disease comprises PRKAG2 cardiac syndrome, and the gene is PRKAG2. In some embodiments, the disease comprises spinocerebellar ataxia and the gene is selected from ATXN1, ATXN2, ATXN3, PLEKHG4, SPTBN2, CACNA1A, ATXN7, ATXN8OS, ATXN10, TTBK2, PPP2R2B, KCNC3, PRKCG, ITPR1, TBP, KCND3, and FGF14. In some embodiments, the disease comprises Usher Syndrome and the gene is selected from MYO7A, USH1C, CDH23, PCDH15, USH1G, USH2A, GPR98, DFNB31, and CLRN1. In some embodiments, the disease comprises von Willebrand disease, and the gene is VWF. In some embodiments, the disease comprises Waardenburg syndrome, and the gene is selected from PAX3, MITF, WS2B, WS2C, SNAI2, EDNRB, EDN3, and SOX10. In some embodiments, the disease comprises Wiskott-Aldrich Syndrome and the gene is WAS. In some embodiments, the disease comprises von Hippel-Lindau disease and the gene is VHL. In some embodiments, the disease comprises Wilson disease and the gene is ATP7B. In some embodiments, the disease comprises Zellweger syndrome and the gene is selected from PEX1, PEX2, PEX3, PEX5, PEX6, PEX10, PEX12, PEX13, PEX14, PEX16, PEX19, and PEX26. In some embodiments, the disease comprises infantile myofibromatosis and the gene is CD34. In some embodiments, the disease comprises platelet glycoprotein IV deficiency and the gene is CD36. In some embodiments, the disease comprises immunodeficiency with hyper-IgM type 3 and the gene is CD40. In some embodiments, the disease comprises hemolytic uremic syndrome and the gene is CD46.

In some embodiments, the disease comprises complement hyperactivation, angiopathic thrombosis, or protein-losing enteropathy and the gene is CD55. In some embodiments, the disease comprises hemolytic anemia and the gene is CD59. In some embodiments, the disease comprises calcification of joints and arteries and the gene is CD73. In some embodiments, the disease comprises immunoglobulin alpha deficiency and the gene is CD79A. In some embodiments, the disease comprises C syndrome and the gene is CD96. In some embodiments, the disease comprises hairy cell leukemia and the gene is CD123. In some embodiments, the disease comprises histiocytic sarcoma and the gene is CD163. In some embodiments, the disease comprises autosomal dominant deafness and the gene is CD164. In some embodiments, the disease comprises immunodeficiency 25 and the gene is CD247. In some embodiments, the disease comprises methymalonic acidemia due to transcobalamin receptor defect and the gene is CD320.

In some embodiments, the disease is pain and the gene is NAV1.7. In some embodiments, the disease is glaucoma and the gene is selected from MYOC and ANGPTL7. In some embodiments, the disease is Tuberous Sclerosis or Subependymal Glioma and the gene is RPTOR. In some embodiments, the disease is Pitt-Hopkins Syndrome and the gene is TCF4.

### Cancer

In some embodiments, the disease is cancer. In some embodiments, the cancer is a solid cancer (i.e., a tumor). In some embodiments, the cancer is selected from a blood cell cancer, a leukemia, and a lymphoma. The cancer can be a leukemia, such as, by way of non-limiting example, acute myeloid (or myelogenous) leukemia (AML), chronic myeloid (or myelogenous) leukemia (CML), acute lymphocytic (or lymphoblastic) leukemia (ALL), and chronic lymphocytic leukemia (CLL). In some embodiments, the cancer is any one of colon cancer, rectal cancer, renal-cell carcinoma, liver cancer, bladder cancer, cancer of the kidney or ureter, lung cancer, non-small cell lung cancer, cancer of the small intestine, esophageal cancer, melanoma, bone cancer, pancreatic cancer, skin cancer, brain cancer (e.g., glioblastoma), cancer of the head or neck, melanoma, uterine cancer, ovarian cancer, breast cancer, testicular cancer, cervical cancer, stomach cancer, Hodgkin's Disease, non-Hodgkin's lymphoma, and thyroid cancer.

In some embodiments, mutations are associated with cancer or are causative of cancer. The target nucleic acid, in some embodiments, comprises a portion of a gene comprising a mutation associated with a disease, such as cancer, a gene whose overexpression is associated with cancer, a tumor suppressor gene, an oncogene, a checkpoint inhibitor gene, a gene associated with cellular growth, a gene associated with cellular metabolism, a gene associated with cell cycle, or a combination thereof. Non-limiting examples of genes comprising a mutation associated with a disease such as cancer are ABL, ACE, AF4/HRX, AKT-2, ALK, ALK/NPM, AML1, AML1/MTG8, APC, ATM, AXIN2, AXL, BAP1, BARD1, BCL-2, BCL-3, BCL-6, BCR/ABL, BLM, BMPR1A, BRCA1, BRCA2, BRIP1, c-MYC, CASR, CCR5, CDC73, CDH1, CDK4, CDKN1B, CDKN1C, CDKN2A, CEBPA, CHEK2, CREBBP, CTNNA1, DBL, DEK/CAN, DICER1, DIS3L2, E2A/PBX1, EGFR, ENL/HRX, EPCAM, ERG/TLS, ERBB, ERBB-2, ETS-1, EWS/FLI-1, FH, FKRP, FLCN, FMS, FOS, FPS, GATA2, GCG, GLI, GPC3, GPGSP, GREM1, HER2/neu, HOX11, HOXB13, HRAS, HST, IL-3, INT-2, JAK1, JUN, KIT, KS3, K-SAM, LBC, LCK, LMO1, LMO2, L-MYC, LYL-1, LYT-10, LYT-10/Cα1, MAS, MAX, MDM-2, MEN1, MET, MITF, MLH1, MLL, MOS, MSH1, MSH2, MSH3, MSH6, MTG8/AML1, MUTYH, MYB, MYH11/CBFB, NBN, NEU, NF1, NF2, N-MYC, NTHL1, OST, PALB2, PAX-5, PBX1/E2A, PCDC1, PDGFRA, PHOX2B, PIM-1, PMS2, POLD1, POLE, POT1, PPARG, PRAD-1, PRKAR1A, PTCH1, PTEN, RAD50, RAD51C, RAD51D, RAF, RAR/PML, RAS-H, RAS-K, RAS-N, RB1, RECQL4, REL/NRG, RET, RHOM1, RHOM2, ROS, RUNX1, SDHA, SDHAF, SDHAF2, SDHB, SDHC, SDHD, SET/CAN, SIS, SKI, SMAD4, SMARCA4, SMARCB1, SMARCE1, SRC, STK11, SUFU, TAL1, TAL2, TAN-1, TIAM1, TERC, TERT, TIMP3, TMEM127, TNF, TP53, TRAC, TSC1, TSC2, TRK, VHL, WRN, and WT1.

Non-limiting examples of oncogenes are KRAS, NRAS, BRAF, MYC, CTNNB1, and EGFR. In some embodiments, the oncogene is a gene that encodes a cyclin dependent kinase (CDK). Non-limiting examples of CDKs are Cdk1, Cdk4, Cdk5, Cdk7, Cdk8, Cdk9, Cdk11 and CDK20. Non-limiting examples of tumor suppressor genes are TP53, RB1, and PTEN

### Donor Nucleic Acids

In some embodiments, a donor nucleic acid comprises a nucleic acid that is incorporated into a target nucleic acid or target sequence. In reference to a viral vector, the term donor nucleic acid refers to a sequence of nucleotides that will be or has been introduced into a cell following transfection of the viral vector. The donor nucleic acid may be introduced into the cell by any mechanism of the transfecting viral vector, including, but not limited to, integration into the genome of the cell or introduction of an episomal plasmid or viral genome via an integration sequence and an integrase. As another example, when used in reference to the activity of an effector protein, the term donor nucleic acid refers to a sequence of nucleotides that will be, or has been, inserted at the site of cleavage by the effector protein (cleaving (hydrolysis of a phosphodiester bond) of a nucleic acid resulting in a nick or double strand break-nuclease activity). As yet another example, when used in reference to homologous recombination, the term donor nucleic acid refers to a sequence of DNA that serves as a template in the process of homologous recombination, which may carry the modification that is to be or has been introduced into the target nucleic acid. By using this donor nucleic acid as a template, the genetic information, including the modification, is copied into the target nucleic acid by way of homologous recombination.

Donor nucleic acids of any suitable size may be integrated into a target nucleic acid or genome. In some embodiments, the donor polynucleotide integrated into a genome is less than 3, about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 kilobases in length. In some embodiments, donor nucleic acids are more than 500 kilobases (kb) in length.

The donor nucleic acid may comprise a sequence that is derived from a plant, bacteria, virus or an animal. The animal may be human. The animal may be a non-human animal, such as, by way of non-limiting example, a mouse, rat, hamster, rabbit, pig, bovine, deer, sheep, goat, chicken, cat, dog, ferret, a bird, non-human primate (e.g., marmoset, rhesus monkey). The non-human animal may be a domesticated mammal or an agricultural mammal.

### Chemical Modifications

Polypeptides (e.g., effector proteins) and nucleic acids (e.g., engineered guide nucleic acids) described herein can be further modified as described throughout and as further described herein. Examples are modifications of interest that do not alter primary sequence, including chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Modifications disclosed herein can also include modification of described polypeptides and/or engineered guide nucleic acids through any suitable method, such as molecular biological techniques and/or synthetic chemistry, to improve their resistance to proteolytic degradation, to change the target sequence specificity, to optimize solubility properties, to alter protein activity (e.g., transcription modulatory activity, enzymatic activity, etc.) or to render them more suitable. Analogs of such polypeptides include those containing residues other than naturally occurring L-amino acids, e.g. D-amino acids or non-naturally occurring synthetic amino acids. D-amino acids may be substituted for some or all of the amino acid residues. Modifications can also include modifications with non-naturally occurring unnatural amino acids. The particular sequence and the manner of preparation will be determined by convenience, economics, purity required, and the like.

Modifications can further include the introduction of various groups to polypeptides and/or engineered guide nucleic acids described herein. For example, groups can be introduced during synthesis or during expression of a polypeptide (e.g., an effector protein), which allow for linking to other molecules or to a surface. Thus, e.g., cysteines can be used to make thioethers, histidines for linking to a metal ion complex, carboxyl groups for forming amides or esters, amino groups for forming amides, and the like.

Modifications can further include modification of nucleic acids described herein (e.g., engineered guide nucleic acids) to provide the nucleic acid with a new or enhanced feature, such as improved stability. Such modifications of a nucleic acid include a base modification, a backbone modification, a sugar modification, or combinations thereof, of one or more nucleotides, nucleosides, or nucleobases in a nucleic acid.

In some embodiments, nucleic acids (e.g., engineered guide nucleic acids) described herein comprise one or more modifications comprising: 2′O-methyl modified nucleotides, 2′ Fluoro modified nucleotides; locked nucleic acid (LNA) modified nucleotides; peptide nucleic acid (PNA) modified nucleotides; nucleotides with phosphorothioate linkages; a 5′ cap (e.g., a 7-methylguanylate cap (m7G)), phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl phosphoramidates, phosphorodiamidates, thionophosphor amidates, thionoalkylphosphonates 5 thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage; phosphorothioate and/or heteroatom internucleoside linkages, such as —CH2-NH—O—CH2-, —CH2-N(CH3)-O—CH2- (known as a methylene (methylimino) or MMI backbone), —CH2-O—N(CH3)-CH2-, —CH2-N(CH3)-N(CH3)-CH2- and —O—N(CH3)-CH2-CH2- (wherein the native phosphodiester internucleotide linkage is represented as —O—P(═O)(OH)—O—CH2-); morpholino linkages (formed in part from the sugar portion of a nucleoside); morpholino backbones; phosphorodiamidate or other non-phosphodiester internucleoside linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methylencimino and methylenchydrazino backbones; sulfonate and sulfonamide backbones; amide backbones; other backbone modifications having mixed N, O, S and CH2 component parts; and combinations thereof.

### Vectors and Multiplexed Expression Vectors

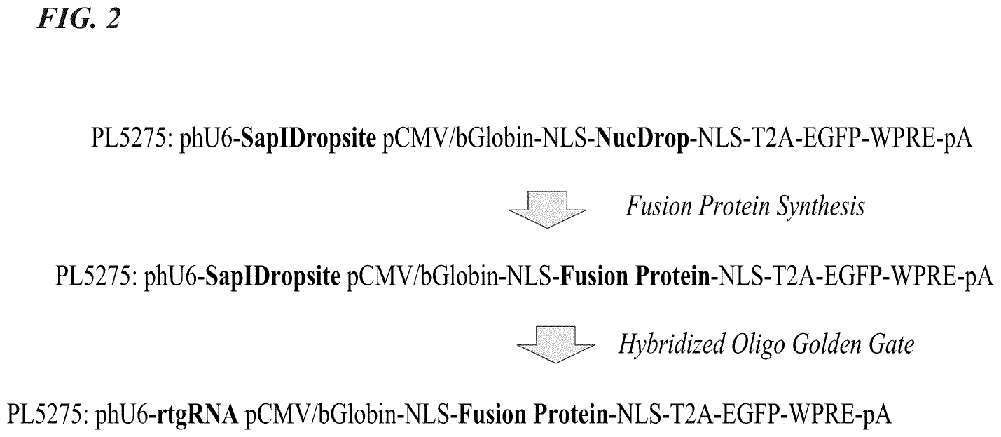

In some embodiments, compositions and systems provided herein comprise a vector system encoding a polypeptide (e.g., an effector protein, an RDDP, and/or a fusion protein thereof) and/or a guide nucleic acid described herein. In some embodiments, compositions and systems provided herein comprise a vector system encoding a guide nucleic acid (e.g., crRNA) described herein. In some embodiments, compositions and systems provided herein comprise a multi-vector system encoding an effector protein, an RDDP, and/or a fusion protein thereof and a guide nucleic acid described herein, wherein the guide nucleic acid and the effector protein, the RDDP, and/or the fusion protein thereof are encoded by the same or different vectors. In some embodiments, the guide and the engineered effector protein are encoded by different vectors of the system. In some embodiments, a nucleic acid encoding a polypeptide (e.g., an effector protein, an RDDP, and/or a fusion protein thereof) comprises an expression vector. In some embodiments, a nucleic acid encoding a polypeptide is a messenger RNA. In some embodiments, an expression vector comprises or encodes an engineered guide nucleic acid. In some embodiments, the expression vector encodes the crRNA. A non-limiting example of a vector design, and vector components which may be oriented in alternative manners, is depicted in FIG. 2.

In some embodiments, a vector can comprise or encode one or more regulatory elements. Regulatory elements can refer to transcriptional and translational control sequences, such as promoters, enhancers, polyadenylation signals, terminators, protein degradation signals, and the like, that provide for and/or regulate transcription of a non-coding sequence or a coding sequence and/or regulate translation of an encoded polypeptide. In some embodiments, a vector can comprise or encode for one or more additional elements, such as, for example, replication origins, antibiotic resistance (or a nucleic acid encoding the same), a tag (or a nucleic acid encoding the same), selectable markers, and the like.

Vectors described herein can encode a promoter—a regulatory region on a nucleic acid, such as a DNA sequence, capable of initiating transcription of a downstream (3′ direction) coding or non-coding sequence. As used herein, a promoter can be bound at its 3′ terminus to a nucleic acid the expression or transcription of which is desired, and extends upstream (5′ direction) to include bases or elements necessary to initiate transcription or induce expression, which could be measured at a detectable level. A promoter can comprise a nucleotide sequence, referred to herein as a “promoter sequence”. A promoter sequence can include a transcription initiation site, and one or more protein binding domains responsible for the binding of transcription machinery, such as RNA polymerase. When eukaryotic promoters are used, such promoters can contain “TATA” boxes and “CAT” boxes. Various promoters, including inducible promoters, may be used to drive expression, i.e., transcriptional activation, of the nucleic acid of interest. Accordingly, in some embodiments, the nucleic acid of interest can be operably linked to a promoter.

Promotors can be any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human H1 promoter (H1). By transcriptional activation, it is intended that transcription will be increased above basal levels in the target cell by 10 fold, by 100 fold, or by 1000 fold, or more. In addition, vectors used for providing a nucleic acid that, when transcribed, produces a guide nucleic acid and/or a nucleic acid that encodes an effector protein to a cell may include nucleic acid sequences that encode for selectable markers in the target cells, so as to identify cells that have taken up the guide nucleic acid and/or an effector protein.

In general, vectors provided herein comprise at least one promotor or a combination of promoters driving expression or transcription of one or more genome editing tools described herein. In some embodiments, the viral vector comprises a nucleotide sequence of a promoter. In some embodiments, the viral vector comprises two promoters. In some embodiments, the viral vector comprises three promoters. In some embodiments, the length of the promoter is less than about 500, less than about 400, or less than about 300 linked nucleotides. In some embodiments, the length of the promoter is at least 100 linked nucleotides. Non-limiting examples of promoters include CMV, 7SK, EF1a, RPBSA, hPGK, EFS, SV40, PGK1, Ubc, human beta actin promoter, CAG, TRE, UAS, Ac5, Polyhedrin, CaMKIIa, GAL1, H1, TEF1, GDS, ADH1, CaMV35S, Ubi, U6, MNDU3, MSCV, MND and CAG.

In some embodiments, the promoter is an inducible promoter that only drives expression of its corresponding gene when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter, as described further in Kulemzin et al., (2019), BMC Med Genomics, 12:44. In some embodiments, the promoter for expressing effector protein is a muscle-specific promoter. In some embodiments, the muscle-specific promoter comprises Ck8e, SPC5-12, or Desmin promoter sequence. In some embodiments, the promoter for expressing effector protein is a ubiquitous promoter. In some embodiments, the ubiquitous promoter comprises MND or CAG promoter sequence.

In some embodiments, an effector protein (or a nucleic acid encoding same) and/or a guide nucleic acid (or a nucleic acid that, when transcribed, produces same) are co-administered with a donor nucleic acid. Coadministration can be contact with a target nucleic acid, administered to a cell, such as a host cell, or administered as method of nucleic acid detection, editing, and/or treatment as described herein, in a single vehicle, such as a single expression vector. In certain embodiments, an effector protein (or a nucleic acid encoding same) and/or a guide nucleic acid (or a nucleic acid that, when transcribed, produces same) are not co-administered with donor nucleic acid in a single vehicle. In certain embodiments, an effector protein (or a nucleic acid encoding same), a guide nucleic acid (or a nucleic acid that, when transcribed, produces same), and/or donor nucleic acid are administered in one or more or two or more vehicles, such as one or more, or two or more expression vectors.

### Viral Vectors

An expression vector can be a viral vector. In some embodiments, a viral vector comprises a nucleic acid to be delivered into a host cell via a recombinantly produced virus or viral particle. The nucleic acid may be single-stranded or double stranded, linear or circular, segmented or non-segmented. The nucleic acid may comprise DNA, RNA, or a combination thereof. In some embodiments, the expression vector is an adeno-associated viral vector. There are a variety of viral vectors that are associated with various types of viruses, including but not limited to retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. A viral vector provided herein can be derived from or based on any such virus. Often the viral vectors provided herein are an adeno-associated viral vector (AAV vector). Generally, an AAV vector has two inverted terminal repeats (ITRs). According, in some embodiments, the viral vector provided herein comprises two inverted terminal repeats of AAV. The DNA sequence in between the ITRs of an AAV vector provided herein may be referred to herein as the sequence encoding the genome editing tools or a transgene. These genome editing tools can include, but are not limited to, an effector protein, effector protein modifications (e.g., nuclear localization signal (NLS), polyA tail), guide nucleic acid(s), respective promoter(s), and a donor nucleic acid, or combinations thereof. In some embodiments, a nuclear localization signal comprises an entity (e.g., peptide) that facilitates localization of a nucleic acid, protein, or small molecule to the nucleus, when present in a cell that contains a nuclear compartment.

In general, viral vectors provided herein comprise at least one promotor or a combination of promoters driving expression or transcription of one or more genome editing tools described herein. In some embodiments, the length of the promoter is less than about 500, less than about 400, or less than about 300 linked nucleotides. In some embodiments, the length of the promoter is at least 100 linked nucleotides. Non-limiting examples of promoters include CMV, EF1a, RPBSA, hPGK, EFS, SV40, PGK1, Ubc, human beta actin promoter, CAG, TRE, UAS, Ac5, Polyhedrin, CaMKIIa, GAL1, H1, TEF1, GDS, ADH1, CaMV35S, Ubi, U6, MNDU3, and MSCV. In some embodiments, the promoter is an inducible promoter that only drives expression of its corresponding gene when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter, as described further in Kulemzin et al., (2019), BMC Med Genomics, 12:44.

In some embodiments, the coding region of the AAV vector forms an intramolecular double-stranded DNA template thereby generating an AAV vector that is a self-complementary AAV (scAAV) vector. In general, the sequence encoding the genome editing tools of an scAAV vector has a length of about 2 kb to about 3 kb. The scAAV vector can comprise nucleotide sequences encoding an effector protein, providing guide nucleic acids described herein, and a donor nucleic acid described herein. In some embodiments, the AAV vector provided herein is a self-inactivating AAV vector.

In some embodiments, an AAV vector provided herein comprises a modification, such as an insertion, deletion, chemical alteration, or synthetic modification, relative to a wild-type AAV vector.

In some embodiments, the viral particle that delivers the viral vector described herein is an AAV. AAVs are characterized by their serotype. Non-limiting examples of AAV serotypes are AAV1, AAV2, AAV3, AAV4, AAV5, AAV6, AAV7, AAV8, AAV9, AAV10, AAV11, AAV12, scAAV, AAV-rh10, chimeric or hybrid AAV, or any combination, derivative, or variant thereof.

### Producing AAV Particles

The AAV particles described herein can be referred to as recombinant AAV (rAAV). Often, rAAV particles are generated by transfecting AAV producing cells with an AAV-containing plasmid carrying the sequence encoding the genome editing tools, a plasmid that carries viral encoding regions, i.e., Rep and Cap gene regions; and a plasmid that provides the helper genes such as E1A, E1B, E2A, E4ORF6 and VA. In some embodiments, the AAV producing cells are mammalian cells. In some embodiments, host cells for rAAV viral particle production are mammalian cells. In some embodiments, a mammalian cell for rAAV viral particle production is a COS cell, a HEK293T cell, a HeLa cell, a KB cell, a derivative thereof, or a combination thereof. In some embodiments, rAAV virus particles can be produced in the mammalian cell culture system by providing the rAAV plasmid to the mammalian cell. In some embodiments, producing rAAV virus particles in a mammalian cell can comprise transfecting vectors that express the rep protein, the capsid protein, and the gene-of-interest expression construct flanked by the ITR sequence on the 5′ and 3′ ends. Methods of such processes are provided in, for example, Naso et al., BioDrugs, 2017 August; 31 (4): 317-334 and Benskey et al., (2019), Methods Mol Biol., 1937:3-26, each of which is incorporated by reference in their entireties.

In some embodiments, rAAV is produced in a non-mammalian cell. In some embodiments, rAAV is produced in an insect cell. In some embodiments, an insect cell for producing rAAV viral particles comprises a Sf9 cell. In some embodiments, production of rAAV virus particles in insect cells can comprise baculovirus. In some embodiments, production of rAAV virus particles in insect cells can comprise infecting the insect cells with three recombinant baculoviruses, one carrying the cap gene, one carrying the rep gene, and one carrying the gene-of-interest expression construct enclosed by an ITR on both the 5′ and 3′ end. In some embodiments, rAAV virus particles are produced by the One Bac system. In some embodiments, rAAV virus particles can be produced by the Two Bac system. In some embodiments, in the Two Bac system, the rep gene and the cap gene of the AAV is integrated into one baculovirus virus genome, and the ITR sequence and the gene-of-interest expression construct is integrated into another baculovirus virus genome. In some embodiments, in the One Bac system, an insect cell line that expresses both the rep protein and the capsid protein is established and infected with a baculovirus virus integrated with the ITR sequence and the gene-of-interest expression construct. Details of such processes are provided in, for example, Smith et. al., (1983), Mol. Cell. Biol., 3 (12): 2156-65; Urabe et al., (2002), Hum. Gene. Ther., 1;13 (16): 1935-43; and Benskey et al., (2019), Methods Mol Biol., 1937:3-26, each of which is incorporated by reference in its entirety.

### Lipid Particles

In some embodiments, compositions and systems provided herein comprise a lipid particle. In some embodiments, a lipid particle is a lipid nanoparticle (LNP). In some embodiments, a lipid or a lipid nanoparticle can encapsulate an expression vector. In some embodiments, a lipid or a lipid nanoparticle can encapsulate the effector protein, the sgRNA or crRNA, the nucleic acid encoding the effector protein and/or the DNA molecule encoding the sgRNA or crRNA. LNPs are effective for delivery of nucleic acids. Beneficial properties of LNP include case of manufacture, low cytotoxicity and immunogenicity, high efficiency of nucleic acid encapsulation and cell transfection, multi-dosing capabilities and flexibility of design (Kulkarni et al., (2018) Nucleic Acid Therapeutics, 28 (3): 146-157). In some embodiments, a method can comprise contacting a cell with an expression vector. In some embodiments, contacting can comprise electroporation, lipofection, or lipid nanoparticle (LNP) delivery of an expression vector.

Methods and Formulations for Introducing Systems and Compositions into a Target Cell

A guide nucleic acid (or a nucleic acid comprising a nucleotide sequence encoding same) and/or an effector protein described herein can be introduced into a host cell by any of a variety of well-known methods. As a non-limiting example, a guide nucleic acid and/or effector protein can be combined with a lipid. As another non-limiting example, a guide nucleic acid and/or effector protein can be combined with a particle or formulated into a particle.

### Methods of Editing

Methods of editing described herein may be employed to generate a genetically modified cell. The cell may be a eukaryotic cell (e.g., a mammalian cell) or a prokaryotic cell (e.g., an archaeal cell). The cell may be derived from a multicellular organism and cultured as a unicellular entity. The cell may comprise a heritable genetic modification, such that progeny cells derived therefrom comprise the heritable genetic mutation. The cell may be progeny of a genetically modified cell comprising a genetic modification of the genetically modified parent cell. A genetically modified cell may comprise a deletion, insertion, mutation, or non-native sequence relative to a wild-type version of the cell or the organism from which the cell was derived.

Methods may comprise contacting a cell or a subject with a fusion protein, effector protein, or partner protein disclosed herein, or any combination thereof. Methods may comprise contacting a cell with a nucleic acid encoding the fusion protein, effector protein, or partner protein, or combination thereof. The nucleic acid may be an expression vector. The nucleic acid may comprise a messenger RNA. Methods may comprise contacting a cell or a subject with an extended guide RNA or a portion thereof. Methods may comprise contacting a cell or a subject with a nucleic acid (e.g., DNA) encoding the extended guide RNA, or a portion thereof.

In some embodiments, the present disclosure provides a mammalian cell comprising a system described herein, e.g., an RDDP described herein (or a fusion protein comprising the same), an effector protein described herein (or a fusion protein comprising the same), and/or an rtgRNA. In some embodiments, the present disclosure provides a mammalian cell comprising one or more polynucleotides encoding one or more components of a system described herein, or one or more vectors comprising the same, e.g., a polynucleotide encoding an RDDP described herein (or a fusion protein comprising the same) or vector comprising the same, a polynucleotide encoding an effector protein described herein (or a fusion protein comprising the same) or a vector comprising the same, and/or a polynucleotide encoding an rtgRNA or a vector comprising the same.

Methods of the disclosure may be performed in a subject. Compositions of the disclosure may be administered to a subject. A subject may be a human. A subject may be a mammal (e.g., rat, mouse, cow, dog, pig, sheep, horse). A subject may be a vertebrate or an invertebrate. A subject may be a laboratory animal. A subject may be a patient. A subject may be at risk of developing, suffering from, or displaying symptoms a disease or disorder as set forth in herein. The subject may have a mutation associated with a gene described herein. The subject may display symptoms associated with a mutation of a gene described herein. In some embodiments, a mutation comprises a point mutation or single nucleotide polymorphism (SNP), a chromosomal mutation, a copy number mutation, or any combination thereof. A point mutation optionally comprises a substitution, insertion, or deletion. In some embodiments, a mutation comprises a chromosomal mutation. A chromosomal mutation can comprise an inversion, a deletion, a duplication, or a translocation. In some embodiments, a mutation comprises a copy number variation. A copy number variation can comprise a gene amplification or an expanding trinucleotide repeat. In some embodiments, mutations may be as described herein.

Methods of the disclosure may be performed in a cell. A cell may be in vitro. A cell may be in vivo. A cell may be ex vivo. A cell may be an isolated cell. A cell may be a cell inside of an organism. A cell may be an organism. A cell may be a cell in a cell culture. A cell may be one of a collection of cells. A cell may be a mammalian cell or derived from a mammalian cell. A cell may be a rodent cell or derived from a rodent cell. A cell may be a human cell or derived from a human cell. A cell may be a eukaryotic cell or derived from a eukaryotic cell. A cell may be a pluripotent stem cell. A cell may be a plant cell or derived from a plant cell. A cell may be an animal cell or derived from an animal cell. A cell may be an invertebrate cell or derived from an invertebrate cell. A cell may be a vertebrate cell or derived from a vertebrate cell.

A cell may be from a specific organ or tissue. Non-limiting examples of organs and tissues from which a cell may be obtained or in which a cell may be located include: muscle, adipose, bone, adrenal gland, pituitary gland, thyroid gland, pancreas, testes, ovaries, uterus, heart, lung, aorta, smooth vasculature, endometrium, brain, neurons, spinal cord, kidney, liver, esophagus, stomach, intestine, colon, bladder, and spleen.

The tissue may be the subject's blood, bone marrow, or cord blood. The tissue may be heterologous donor blood, cord blood, or bone marrow. The tissue may be allogenic blood, cord blood, or bone marrow. In some embodiments, the cell is a stem cell. Non-limiting examples of stem cells are hematopoietic stem cells, muscle stem cells (also referred to as myoblasts or muscle progenitor cells), and pluripotent stem cells (including induced pluripotent stem cells). In some embodiments, the cell is cell derived or differentiated from a pluripotent stem cell. In some embodiments, the cell is a hepatocyte.

In some instances, the cell is an immune cell. Non-limiting examples of immune cells are lymphocytes (T cells, B cells, and NK cells), neutrophils, and monocytes/macrophages.

### Pharmaceutical Compositions and Modes of Administration

Disclosed herein, in some aspects, are pharmaceutical compositions for modifying a target nucleic acid in a cell or a subject, comprising any one of the effector proteins, engineered effector proteins, fusion effector proteins, or guide nucleic acids as described herein and any combination thereof. Also disclosed herein, in some aspects, are pharmaceutical compositions comprising a nucleic acid encoding any one of the effector proteins, engineered effector proteins, fusion effector proteins, or guide nucleic acids as described herein and any combination thereof. In some embodiments, pharmaceutical compositions comprise a plurality of guide nucleic acids. Pharmaceutical compositions may be used to modify a target nucleic acid or the expression thereof in a cell in vitro, in vivo or ex vivo.

In some embodiments, pharmaceutical compositions comprise one or more nucleic acids encoding an effector protein, fusion effector protein, fusion partner, a guide nucleic acid, or a combination thereof; and a pharmaceutically acceptable carrier or diluent. The effector protein, fusion effector protein, fusion partner protein, or combination thereof may be any one of those described herein. The one or more nucleic acids may comprise a plasmid. The one or more nucleic acids may comprise a nucleic acid expression vector. The one or more nucleic acids may comprise a viral vector. In some embodiments, the viral vector is a lentiviral vector. In some embodiments, the vector is an adeno-associated viral (AAV) vector. In some embodiments, compositions, including pharmaceutical compositions, comprise a viral vector encoding a fusion effector protein and a guide nucleic acid, wherein at least a portion of the guide nucleic acid binds to the effector protein of the fusion effector protein.

In some embodiments, pharmaceutical compositions comprise a virus comprising a viral vector encoding a fusion effector protein, an effector protein, a fusion partner, a guide nucleic acid, or a combination thereof; and a pharmaceutically acceptable carrier or diluent. The virus may be a lentivirus. The virus may be an adenovirus. The virus may be a non-replicating virus. The virus may be an adeno-associated virus (AAV). The viral vector may be a retroviral vector. Retroviral vectors may include gamma-retroviral vectors such as vectors derived from the Moloney Murine Leukemia Virus (MoMLV, MMLV, MuLV, or MLV) or the Murine Stem cell Virus (MSCV) genome. Retroviral vectors may include lentiviral vectors such as those derived from the human immunodeficiency virus (HIV) genome. In some embodiments, the viral vector is a chimeric viral vector, comprising viral portions from two or more viruses. In some embodiments, the viral vector is a recombinant viral vector.

In some embodiments, the viral vector is an AAV. The AAV may be any AAV known in the art. In some embodiments, the viral vector corresponds to a virus of a specific serotype. In some examples, the serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, and an AAV12 serotype. In some embodiments the AAV vector is a recombinant vector, a hybrid AAV vector, a chimeric AAV vector, a self-complementary AAV (scAAV) vector, a single-stranded AAV or any combination thereof. scAAV genomes are generally known in the art and contain both DNA strands which can anneal together to form double-stranded DNA.

In some embodiments, methods of producing delivery vectors herein comprise packaging a nucleic acid, or transgene, encoding an effector protein and a nucleic acid that, when transcribed, produces a guide nucleic acid, or a combination thereof, into an AAV vector. In some embodiments, methods of producing the delivery vector comprises, (a) contacting a cell with at least one nucleic acid, or transgene that, when transcribed, produces a guide nucleic acid; at least one nucleic acid that encodes: (i) a Replication (Rep) gene; and (ii) a Capsid (Cap) gene that encodes an AAV capsid protein; (b) expressing the AAV capsid protein in the cell; (c) assembling an AAV particle; and (d) packaging a Cas effector encoding nucleic acid into the AAV particle, thereby generating an AAV delivery vector. In some embodiments, promoters, stuffer sequences, and any combination thereof may be packaged in the AAV vector. In some embodiments, the AAV vector comprises a sequence encoding a guide nucleic acid. In some embodiments, the guide nucleic acid comprises a crRNA. In some embodiments, the guide nucleic acid is a crRNA. In some embodiments, the guide nucleic acid comprises a sgRNA. In some embodiments, the guide nucleic acid is a sgRNA. In some examples, the AAV vector can package 1, 2, 3, 4, or 5 nucleotide sequences encoding guide nucleic acids or copies thereof. In some examples, the AAV vector packages 1 or 2 nucleotide sequences encoding guide nucleic acids or copies thereof. In some embodiments, the AAV vector packages a nucleotide sequence encoding a first guide nucleic acid and a nucleotide sequence encoding a second guide nucleic acid, wherein the first guide nucleic acid and the second guide nucleic acid are the same. In some embodiments, the AAV vector packages a nucleotide sequence encoding a first guide nucleic acid and a nucleotide sequence encoding a second guide nucleic acid, wherein the first guide nucleic acid and the second guide nucleic acid are different. In some embodiments, the AAV vector comprises inverted terminal repeats, e.g., a 5′ inverted terminal repeat and a 3′ inverted terminal repeat. In some embodiments, the inverted terminal repeat comprises inverted terminal repeats from AAV. In some embodiments, the inverted terminal repeat comprises inverted terminal repeats of ssAAV vector or scAAV vector. In some embodiments, the AAV vector comprises a mutated inverted terminal repeat that lacks a terminal resolution site.

In some embodiments, a hybrid AAV vector is produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV9), wherein the first and second AAV serotypes may be not the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

In some embodiments, the AAV vector may be a chimeric AAV vector. In some embodiments, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some examples, the delivery vector may be a eukaryotic vector, a prokaryotic vector (e.g., a bacterial vector) a viral vector, or any combination thereof. In some embodiments, the delivery vehicle may be a non-viral vector. In some embodiments, the delivery vehicle may be a plasmid. In some embodiments, the plasmid comprises DNA. In some embodiments, the plasmid comprises RNA. In some examples, the plasmid comprises circular double-stranded DNA. In some examples, the plasmid may be linear. In some examples, the plasmid comprises one or more genes of interest and one or more regulatory elements. In some examples, the plasmid comprises a bacterial backbone containing an origin of replication and an antibiotic resistance gene or other selectable marker for plasmid amplification in bacteria. In some examples, the plasmid may be a minicircle plasmid. In some examples, the plasmid contains one or more genes that provide a selective marker to induce a target cell to retain the plasmid. In some examples, the plasmid may be formulated for delivery through injection by a needle carrying syringe. In some examples, the plasmid may be formulated for delivery via electroporation. In some examples, the plasmids may be engineered through synthetic or other suitable means known in the art. For example, in some embodiments, the genetic elements may be assembled by restriction digest of the desired genetic sequence from a donor plasmid or organism to produce ends of the DNA which may then be readily ligated to another genetic sequence.

In some embodiments, the vector is a non-viral vector, and a physical method or a chemical method is employed for delivery into the somatic cell. Exemplary physical methods include electroporation, gene gun, sonoporation, magnetofection, or hydrodynamic delivery. Exemplary chemical methods include delivery of the recombinant polynucleotide via liposomes such as, cationic lipids or neutral lipids; dendrimers; nanoparticles; or cell-penetrating peptides.

In some embodiments, a fusion effector protein as described herein is inserted into a vector. In some embodiments, the vector comprises a transgene which comprises a nucleotide sequence of one or more promoters, enhancers, ribosome binding sites, RNA splice sites, polyadenylation sites, a replication origin, and/or transcriptional terminator sequences.

In some embodiments, the AAV vector comprises a self-processing array system for guide nucleic acid. Such a self-processing array system refers to a system for multiplexing, stringing together multiple guide nucleic acids under the control of a single promoter. In general, plasmids and vectors described herein comprise at least one promoter. In some embodiments, the promoters are constitutive promoters. In other embodiments, the promoters are inducible promoters. In additional embodiments, the promoters are prokaryotic promoters (e.g., drive expression of a gene in a prokaryotic cell). In some embodiments, the promoters are eukaryotic promoters, (e.g., drive expression of a gene in a eukaryotic cell). Exemplary promoters include, but are not limited to, CMV, EF1a, SV40, PGK1, Ubc, human beta actin, CAG, TRE, UAS, Ac5, polyhedron, CaMKIIa, GAL1-10, TEF1, GDS, ADH1, CaMV35S, Ubi, H1, U6, CaMV35S, SV40, CMV, 7SK, and HSV TK promoter. In some embodiments, the promoter is CMV. In some embodiments, the promoter is EF1a. In some embodiments, the promoter is U6. In some embodiments, the promote is H1. In some embodiments, the promoter is 7SK. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, the AAV vector comprises a promoter for expressing effector proteins. In some embodiments, the promoter for expressing effector protein is a site-specific promoter. In some embodiments, the promoter for expressing effector protein is a muscle-specific promoter. In some embodiments, the muscle-specific promoter comprises Ck8e, SPC5-12, or Desmin promoter sequence. In some embodiments, the promoter for expressing effector protein is a ubiquitous promoter. In some embodiments, the ubiquitous promoter comprises MND or CAG promoter sequence.

In some embodiments, the AAV vector comprises a stuffer sequence. A stuffer sequence can refer to a non-coding sequence of nucleotides that adjusts the length of the viral genome when inserted into a vector to increase packaging efficiency, increase overall viral titer during production, increase transfection efficacy, increase transfection efficiency, and/or decrease vector toxicity. In some embodiments, the stuffer sequence comprises 5′ untranslated region, 3′ untranslated region or combination thereof. In some embodiments, a stuffer sequence serves no other functional purpose than to increase the length of the viral genome. In some embodiments, a stuffer sequence may increase the length of the viral genome as well as have other functional elements

In some embodiments, the 3′-untranslated region comprises a nucleotide sequence of an intron. In some embodiments, the 3′-untranslated region comprises one or more sequence elements, such as an intron sequence or an enhancer sequence. In some embodiments, the 3′-untranslated region comprises an enhancer. In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R-U5′ segment in LTR of HTLV-I (Mol. Cell. Biol., Vol. 8 (1), p. 466-472, 1988); SV40 enhancer; the intron sequence between exons 2 and 3 of rabbit β-globin (Proc. Natl. Acad. Sci. USA, Vol. 78 (3), p. 1527-31, 1981); and the genome region of human growth hormone (J Immunol., Vol. 155 (3), p. 1286-95, 1995). In some embodiments, the enhancer is WPRE.

In some embodiments, the AAV vector comprises one or more polyadenylation (poly A) signal sequences. In some embodiments, the polyadenlyation signal sequence comprises hGH poly A signal sequence. In some embodiments, the polyadenlyation signal sequence comprises sv40 poly A signal sequence.

Pharmaceutical compositions described herein may comprise a salt. In some embodiments, the salt is a sodium salt. In some embodiments, the salt is a potassium salt. In some embodiments, the salt is a magnesium salt. In some embodiments, the salt is NaCl. In some embodiments, the salt is KNO₃. In some embodiments, the salt is Mg²⁺SO₄²⁻.

Non-limiting examples of pharmaceutically acceptable carriers and diluents suitable for the pharmaceutical compositions disclosed herein include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathionc); adjuvants (e.g., aluminum hydroxide); surfactants (Polysorbate 80, Polysorbate 20, or Pluronic F68); glycerol; sorbitol; mannitol; polyethyleneglycol; and preservatives.

In some embodiments, pharmaceutical compositions are in the form of a solution (e.g., a liquid). The solution may be formulated for injection, e.g., intravenous or subcutaneous injection. In some embodiments, the pH of the solution is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some embodiments, the pH of the solution is less than 7. In some embodiments, the pH is greater than 7.

In some embodiments, pharmaceutical compositions comprise an: effector protein, fusion effector protein, fusion partner, a guide nucleic acid, or a combination thereof; and a pharmaceutically acceptable carrier or diluent. In some embodiments, pharmaceutical compositions comprise one or more nucleic acids encoding an: effector protein, fusion effector protein, fusion partner, a guide nucleic acid, or a combination thereof; and a pharmaceutically acceptable carrier or diluent.

In some embodiments, guide nucleic acid can be a plurality of guide nucleic acids. In some embodiments, the effector protein comprises a sequence that is at least 65%, at least 70%, at least 75%, at least 80%, at least 85%, at least 90%, at least 95%, at least 97%, or at least 98%, at least 99%, or 100% identical to any one of the effector sequences of TABLE 1. In some embodiments, the guide nucleic acid comprises a nucleotide sequence of any one of the guide sequences of TABLE 1 or any combination thereof. In some embodiments, the PAM nucleic acid comprises a nucleotide sequence of any one of the PAM sequences of TABLE 1 or any combination thereof.

In some embodiments, the methods of this invention include using an inhibitor of DNA synthesis. In some embodiments the inhibitor is an inhibitor of replication fork regression. In some embodiments the inhibitor is an inhibitor of single strand break repair. In some embodiments the inhibitor is an inhibitor of double-strand break repair. In some embodiments the inhibitor is an inhibitor of non-homologous enjoining. In some embodiments the inhibitor is an inhibitor of RAD51. In some embodiments the inhibitor is AZD7648.

### Sequences and Tables

TABLE 1
Exemplary Effector Protein Amino Acid Sequences, Exemplary Guide Repeat Sequences, and
Exemplary PAM Sequences
TABLE 1 provides illustrative amino acid sequences of effector proteins, guide sequences, and
PAM sequences that are useful in the compositions, systems and methods described herein. With
regards to the PAM sequences: N is any nucleotide, V is A, C, or G; B is C, G, or T; and S
is G or C.

| | | | Exemplary | | |
|---|---|---|---|---|---|
| | | SEQ | Guide Repeat | SEQ | Exemplary |
| Name | Exemplary Effector Sequence | ID: | sequence | ID: | PAM Sequence |
| CasΦ.1 | MADTPTLFTQFLRHHLPGQRFRKDILKQAGRILANKGEDA | 1 | GGAGAGAUCUCA | 135 | |
| | TIAFLRGKSEESPPDFQPPVKCPIIACSRPLTEWPIYQAS | | AACGAUUGCUCG | | |
| | VAIQGYVYGQSLAEFEASDPGCSKDGLLGWFDKTGVCTDY | | AUUAGUCGAGAC | | |
| | FSVQGLNLIFQNARKRYIGVQTKVINRNEKRHKKLKRINA | | | | |
| | KRIAEGLPELTSDEPESALDETGHLIDPPGLNTNIYCYQQ | | | | |
| | VSPKPLALSEVNQLPTAYAGYSTSGDDPIQPMVTKDRLSI | | | | |
| | SKGQPGYIPEHQRALLSQKKHRRMRGYGLKARALLVIVRI | | | | |
| | QDDWAVIDLRSLLRNAYWRRIVQTKEPSTITKLLKLVTGD | | | | |
| | PVLDATRMVATFTYKPGIVQVRSAKCLKNKQGSKLFSERY | | | | |
| | LNETVSVTSIDLGSNNLVAVATYRLVNGNTPELLQRFTLP | | | | |
| | SHLVKDFERYKQAHDTLEDSIQKTAVASLPQGQQTEIRMW | | | | |
| | SMYGFREAQERVCQELGLADGSIPWNVMTATSTILTDLFL | | | | |
| | ARGGDPKKCMFTSEPKKKKNSKQVLYKIRDRAWAKMYRTL | | | | |
| | LSKETREAWNKALWGLKRGSPDYARLSKRKEELARRCVNY | | | | |
| | TISTAEKRAQCGRTIVALEDLNIGFFHGRGKQEPGWVGLF | | | | |
| | TRKKENRWLMQALHKAFLELAHHRGYHVIEVNPAYTSQTC | | | | |
| | PVCRHCDPDNRDQHNREAFHCIGCGFRGNADLDVATHNIA | | | | |
| | MVAITGESLKRARGSVASKTPQPLAAE | | | | |
| CasΦ.2 | MPKPAVESEFSKVLKKHFPGERFRSSYMKRGGKILAAQGE | 2 | GUCGGAACGCUC | 136 | |
| | EAVVAYLQGKSEEEPPNFQPPAKCHVVTKSRDFAEWPIMK | | AACGAUUGCCCC | | |
| | ASEAIQRYIYALSTTERAACKPGKSSESHAAWFAATGVSN | | UCACGAGGGGAC | | |
| | HGYSHVQGLNLIFDHTLGRYDGVLKKVQLRNEKARARLES | | | | |
| | INASRADEGLPEIKAEEEEVATNETGHLLQPPGINPSFYV | | | | |
| | YQTISPQAYRPRDEIVLPPEYAGYVRDPNAPIPLGVVRNR | | | | |
| | CDIQKGCPGYIPEWQREAGTAISPKTGKAVTVPGLSPKKN | | | | |
| | KRMRRYWRSEKEKAQDALLVTVRIGTDWVVIDVRGLLRNA | | | | |
| | RWRTIAPKDISLNALLDLFTGDPVIDVRRNIVTFTYTLDA | | | | |
| | CGTYARKWTLKGKQTKATLDKLTATQTVALVAIDLGQTNP | | | | |
| | ISAGISRVTQENGALQCEPLDRFTLPDDLLKDISAYRIAW | | | | |
| | DRNEEELRARSVEALPEAQQAEVRALDGVSKETARTQLCA | | | | |
| | DFGLDPKRLPWDKMSSNTTFISEALLSNSVSRDQVFFTPA | | | | |
| | PKKGAKKKAPVEVMRKDRTWARAYKPRLSVEAQKLKNEAL | | | | |
| | WALKRTSPEYLKLSRRKEELCRRSINYVIEKTRRRTQCQI | | | | |
| | VIPVIEDLNVRFFHGSGKRLPGWDNFFTAKKENRWFIQGL | | | | |
| | HKAFSDLRTHRSFYVFEVRPERTSITCPKCGHCEVGNRDG | | | | |
| | EAFQCLSCGKTCNADLDVATHNLTQVALTGKTMPKREEPR | | | | |
| | DAQGTAPARKTKKASKSKAPPAEREDQTPAQEPSQTS | | | | |
| CasΦ.3 | MYILEMADLKSEPSLLAKLLRDRFPGKYWLPKYWKLAEKK | 3 | | | |
| | RLTGGEEAACEYMADKQLDSPPPNFRPPARCVILAKSRPF | | | | |
| | EDWPVHRVASKAQSFVIGLSEQGFAALRAAPPSTADARRD | | | | |
| | WLRSHGASEDDLMALEAQLLETIMGNAISLHGGVLKKIDN | | | | |
| | ANVKAAKRLSGRNEARLNKGLQELPPEQEGSAYGADGLLV | | | | |
| | NPPGLNLNIYCRKSCCPKPVKNTARFVGHYPGYLRDSDSI | | | | |
| | LISGTMDRLTIIEGMPGHIPAWQREQGLVKPGGRRRRLSG | | | | |
| | SESNMRQKVDPSTGPRRSTRSGTVNRSNQRTGRNGDPLLV | | | | |
| | EIRMKEDWVLLDARGLLRNLRWRESKRGLSCDHEDLSLSG | | | | |
| | LLALFSGDPVIDPVRNEVVFLYGEGIIPVRSTKPVGTRQS | | | | |
| | KKLLERQASMGPLTLISCDLGQTNLIAGRASAISLTHGSL | | | | |
| | GVRSSVRIELDPEIIKSFERLRKDADRLETEILTAAKETL | | | | |
| | SDEQRGEVNSHEKDSPQTAKASLCRELGLHPPSLPWGQMG | | | | |
| | PSTTFIADMLISHGRDDDAFLSHGEFPTLEKRKKFDKRFC | | | | |
| | LESRPLLSSETRKALNESLWEVKRTSSEYARLSQRKKEMA | | | | |
| | RRAVNFVVEISRRKTGLSNVIVNIEDLNVRIFHGGGKQAP | | | | |
| | GWDGFFRPKSENRWFIQAIHKAFSDLAAHHGIPVIESDPQ | | | | |
| | RTSMTCPECGHCDSKNRNGVRFLCKGCGASMDADFDAACR | | | | |
| | NLERVALTGKPMPKPSTSCERLLSATTGKVCSDHSLSHDA | | | | |
| | IEKAS | | | | |
| CasΦ.4 | MEKEITELTKIRREFPNKKFSSTDMKKAGKLLKAEGPDAV | 4 | ACCAAAACGACU | 137 | NTYN |
| | RDFLNSCQEIIGDFKPPVKTNIVSISRPFEEWPVSMVGRA | | AUUGAUUGCCCA | | |
| | IQEYYFSLTKEELESVHPGTSSEDHKSFFNITGLSNYNYT | | GUACGCUGGGAC | | |
| | SVQGLNLIFKNAKAIYDGTLVKANNKNKKLEKKFNEINHK | | | | |
| | RSLEGLPIITPDFEEPFDENGHLNNPPGINRNIYGYQGCA | | | | |
| | AKVFVPSKHKMVSLPKEYEGYNRDPNLSLAGERNRLEIPE | | | | |
| | GEPGHVPWFQRMDIPEGQIGHVNKIQRFNFVHGKNSGKVK | | | | |
| | FSDKTGRVKRYHHSKYKDATKPYKFLEESKKVSALDSILA | | | | |
| | IITIGDDWVVFDIRGLYRNVFYRELAQKGLTAVQLLDLFT | | | | |
| | GDPVIDPKKGVVTFSYKEGVVPVFSQKIVPRFKSRDTLEK | | | | |
| | LTSQGPVALLSVDLGQNEPVAARVCSLKNINDKITLDNSC | | | | |
| | RISFLDDYKKQIKDYRDSLDELEIKIRLEAINSLETNQQV | | | | |
| | EIRDLDVFSADRAKANTVDMFDIDPNLISWDSMSDARVST | | | | |
| | QISDLYLKNGGDESRVYFEINNKRIKRSDYNISQLVRPKL | | | | |
| | SDSTRKNLNDSIWKLKRTSEEYLKLSKRKLELSRAVVNYT | | | | |
| | IRQSKLLSGINDIVIILEDLDVKKKENGRGIRDIGWDNFF | | | | |
| | SSRKENRWFIPAFHKAFSELSSNRGLCVIEVNPAWTSATC | | | | |
| | PDCGFCSKENRDGINFTCRKCGVSYHADIDVATLNIARVA | | | | |
| | VLGKPMSGPADRERLGDTKKPRVARSRKTMKRKDISNSTV | | | | |
| | EAMVTA | | | | |
| CasΦ.5 | MDMLDTETNYATETPAQQQDYSPKPPKKAQRAPKGFSKKA | 5 | | | |
| | RPEKKPPKPITLFTQKHFSGVRFLKRVIRDASKILKLSES | | | | |
| | RTITFLEQAIERDGSAPPDVTPPVHNTIMAVTRPFEEWPE | | | | |
| | VILSKALQKHCYALTKKIKIKTWPKKGPGKKCLAAWSART | | | | |
| | KIPLIPGQVQATNGLEDRIGSIYDGVEKKVTNRNANKKLE | | | | |
| | YDEAIKEGRNPAVPEYETAYNIDGTLINKPGYNPNLYITQ | | | | |
| | SRTPRLITEADRPLVEKILWQMVEKKTQSRNQARRARLEK | | | | |
| | AAHLQGLPVPKFVPEKVDRSQKIEIRIIDPLDKIEPYMPQ | | | | |
| | DRMAIKASQDGHVPYWQRPFLSKRRNRRVRAGWGKQVSSI | | | | |
| | QAWLTGALLVIVRLGNEAFLADIRGALRNAQWRKLLKPDA | | | | |
| | TYQSLFNLFTGDPVVNTRINHLTMAYREGVVNIVKSRSFK | | | | |
| | GRQTREHLLTLLGQGKTVAGVSFDLGQKHAAGLLAAHFGL | | | | |
| | GEDGNPVFTPIQACFLPQRYLDSLINYRNRYDALTLDMRR | | | | |
| | QSLLALTPAQQQEFADAQRDPGGQAKRACCLKLNLNPDEI | | | | |
| | RWDLVSGISTMISDLYIERGGDPRDVHQQVETKPKGKRKS | | | | |
| | EIRILKIRDGKWAYDFRPKIADETRKAQREQLWKLQKASS | | | | |
| | EFERLSRYKINIARAIANWALQWGRELSGCDIVIPVLEDL | | | | |
| | NVGSKFFDGKGKWLLGWDNRFTPKKENRWFIKVLHKAVAE | | | | |
| | LAPHRGVPVYEVMPHRTSMTCPACHYCHPTNREGDRFECQ | | | | |
| | SCHVVKNTDRDVAPYNILRVAVEGKTLDRWQAEKKPQAEP | | | | |
| | DRPMILIDNQES | | | | |
| CasΦ.6 | MDMLDTETNYATETPAQQQDYSPKPPKKAQRAPKGFSKKA | 6 | | | |
| | RPEKKPPKPITLFTQKHFSGVRFLKRVIRDASKILKLSES | | | | |
| | RTITFLEQAIERDGSAPPDVTPPVHNTIMAVTRPFEEWPE | | | | |
| | VILSKALQKHCYALTKKIKIKTWPKKGPGKKCLAAWSART | | | | |
| | KIPLIPGQVQATNGLFDRIGSIYDGVEKKVTNRNANKKLE | | | | |
| | YDEAIKEGRNPAVPEYETAYNIDGTLINKPGYNPNLYITQ | | | | |
| | SRTPRLITEADRPLVEKILWQMVEKKTQSRNQARRARLEK | | | | |
| | AAHLQGLPVPKFVPEKVDRSQKIEIRIIDPLDKIEPYMPQ | | | | |
| | DRMAIKASQDGHVPYWQRPFLSKRRNRRVRAGWGKQVSSI | | | | |
| | QAWLTGALLVIVRLGNEAFLADIRGALRNAQWRKLLKPDA | | | | |
| | TYQSLFNLFTGDPVVNTRINHLTMAYREGVVDIVKSRSFK | | | | |
| | GRQTREHLLTLLGQGKTVAGVSFDLGQKHAAGLLAAHFGL | | | | |
| | GEDGNPVFTPIQACFLPQRYLDSLINYRNRYDALTLDMRR | | | | |
| | QSLLALTPAQQQEFADAQRDPGGQAKRACCLKLNLNPDEI | | | | |
| | RWDLVSGISTMISDLYIERGGDPRDVHQQVETKPKGKRKS | | | | |
| | EIRILKIRDGKWAYDFRPKIADETRKAQREQLWKLQKASS | | | | |
| | EFERLSRYKINIARAIANWALQWGRELSGCDIVIPVLEDL | | | | |
| | NVGSKFFDGKGKWLLGWDNRFTPKKENRWFIKVLHKAVAE | | | | |
| | LAPHKGVPVYEVMPHRTSMTCPACHYCHPTNREGDRFECQ | | | | |
| | SCHVVKNTDRDVAPYNILRVAVEGKTLDRWQAEKKPQAEP | | | | |
| | DRPMILIDNQES | | | | |
| CasΦ.7 | MSSLPTPLELLKQKHADLFKGLQFSSKDNKMAGKVLKKDG | 7 | | | |
| | EEAALAFLSERGVSRGELPNFRPPAKTLVVAQSRPFEEFP | | | | |
| | IYRVSEAIQLYVYSLSVKELETVPSGSSTKKEHQRFFQDS | | | | |
| | SVPDFGYTSVQGLNKIFGLARGIYLGVITRGENQLQKAKS | | | | |
| | KHEALNKKRRASGEAETEFDPTPYEYMTPERKLAKPPGVN | | | | |
| | HSIMCYVDISVDEFDERNPDGIVLPSEYAGYCREINTAIE | | | | |
| | KGTVDRLGHLKGGPGYIPGHQRKESTTEGPKINFRKGRIR | | | | |
| | RSYTALYAKRDSRRVRQGKLALPSYRHHMMRLNSNAESAI | | | | |
| | LAVIFFGKDWVVFDLRGLLRNVRWRNLFVDGSTPSTLLGM | | | | |
| | FGDPVIDPKRGVVAFCYKEQIVPVVSKSITKMVKAPELLN | | | | |
| | KLYLKSEDPLVLVAIDLGQTNPVGVGVYRVMNASLDYEVV | | | | |
| | TRFALESELLREIESYRQRTNAFEAQIRAETFDAMTSEEQ | | | | |
| | EEITRVRAFSASKAKENVCHRFGMPVDAVDWATMGSNTIH | | | | |
| | IAKWVMRHGDPSLVEVLEYRKDNEIKLDKNGVPKKVKLTD | | | | |
| | KRIANLTSIRLRFSQETSKHYNDTMWELRRKHPVYQKLSK | | | | |
| | SKADFSRRVVNSIIRRVNHLVPRARIVFIIEDLKNLGKVF | | | | |
| | HGSGKRELGWDSYFEPKSENRWFIQVLHKAFSETGKHKGY | | | | |
| | YIIECWPNWTSCTCPKCSCCDSENRHGEVERCLACGYTCN | | | | |
| | TDFGTAPDNLVKIATTGKGLPGPKKRCKGSSKGKNPKIAR | | | | |
| | SSETGVSVTESGAPKVKKSSPTQTSQSSSQSAP | | | | |
| CasΦ.8 | MNKIEKEKTPLAKLMNENFAGLRFPFAIIKQAGKKLLKEG | 8 | | | |
| | ELKTIEYMTGKGSIEPLPNFKPPVKCLIVAKRRDLKYFPI | | | | |
| | CKASCEIQSYVYSLNYKDFMDYFSTPMTSQKQHEEFFKKS | | | | |
| | GLNIEYQNVAGLNLIFNNVKNTYNGVILKVKNRNEKLKKK | | | | |
| | AIKNNYEFEEIKTENDDGCLINKPGINNVIYCFQSISPKI | | | | |
| | LKNITHLPKEYNDYDCSVDRNIIQKYVSRLDIPESQPGHV | | | | |
| | PEWQRKLPEFNNTNNPRRRRKWYSNGRNISKGYSVDQVNQ | | | | |
| | AKIEDSLLAQIKIGEDWIILDIRGLLRDLNRRELISYKNK | | | | |
| | LTIKDVLGFFSDYPIIDIKKNLVTFCYKEGVIQVVSQKSI | | | | |
| | GNKKSKQLLEKLIENKPIALVSIDLGQTNPVSVKISKLNK | | | | |
| | INNKISIESFTYRFLNEEILKEIEKYRKDYDKLELKLINE | | | | |
| | A | | | | |
| CasΦ.9 | MDMLDTETNYATETPSQQQDYSPKPPKKDRRAPKGFSKKA | 9 | | | |
| | RPEKKPPKPITLFTQKHFSGVRFLKRVIRDASKILKLSES | | | | |
| | RTITFLEQAIERDGSAPPDVTPPVHNTIMAVTRPFEEWPE | | | | |
| | VILSKALQKHCYALTKKIKIKTWPKKGPGKKCLAAWSART | | | | |
| | KIPLIPGQVQATNGLFDRIGSIYDGVEKKVTNRNANKKLE | | | | |
| | YDEAIKEGRNPAVPEYETAYNIDGTLINKPGYNPNLYITQ | | | | |
| | SRTPRLITEADRPLVEKILWQMVEKKTQSRNQARRARLEK | | | | |
| | AAHLQGLPVPKFVPEKVDRSQKIEIRIIDPLDKIEPYMPQ | | | | |
| | DRMAIKASQDGHVPYWQRPFLSKRRNRRVRAGWGKQVSSI | | | | |
| | QAWLTGALLVIVRLGNEAFLADIRGALRNAQWRKLLKPDA | | | | |
| | TYQSLFNLFTGDPVVNTRTNHLTMAYREGVVDIVKSRSFK | | | | |
| | GRQTREHLLTLLGQGKTVAGVSFDLGQKHAAGLLAAHFGL | | | | |
| | GEDGNPVFTPIQACFLPQRYLDSLINYRNRYDALTLDMRR | | | | |
| | QSLLALTPAQQQEFADAQRDPGGQAKRACCLKLNLNPDEI | | | | |
| | RWDLVSGISTMISDLYIERGGDPRDVHQQVETKPKGKRKS | | | | |
| | EIRILKIRDGKWAYDFRPKIADETRKAQREQLWKLQKASS | | | | |
| | EFERLSRYKINIARAIANWALQWGRELSGCDIVIPVLEDL | | | | |
| | NVGSKFFDGKGKWLLGWDNRFTPKKENRWFIKVLHKAVAE | | | | |
| | LAPHRGVPVYEVMPHRTSMTCPACHYCHPTNREGDRFECQ | | | | |
| | SCHVVKNTDRDVAPYNILRVAVEGKTLDRWQAEKKPQAEP | | | | |
| | DRPMILIDNQES | | | | |
| CasΦ.10 | MDMLDTETNYATETPSQQQDYSPKPPKKDRRAPKGFSKKA | 10 | GGAUCUGAGGAU | 138 | |
| | RPEKKPPKPITLFTQKHFSGVRFLKRVIRDASKILKLSES | | CAUUAUUGCUCG | | |
| | RTITFLEQAIERDGSAPPDVTPPVHNTIMAVTRPFEEWPE | | UUACGACGAGAC | | |
| | VILSKALQKHCYALTKKIKIKTWPKKGPGKKCLAAWSART | | | | |
| | KIPLIPGQVQATNGLFDRIGSIYDGVEKKVTNRNANKKLE | | | | |
| | YDEAIKEGRNPAVPEYETAYNIDGTLINKPGYNPNLYITQ | | | | |
| | SRTPRLITEADRPLVEKILWQMVEKKTQSRNQARRARLEK | | | | |
| | AAHLQGLPVPKFVPEKVDRSQKIEIRIIDPLDKIEPYMPQ | | | | |
| | DRMAIKASQDGHVPYWQRPFLSKRRNRRVRAGWGKQVSSI | | | | |
| | QAWLTGALLVIVRLGNEAFLADIRGALRNAQWRKLLKPDA | | | | |
| | TYQSLFNLFTGDPVVNTRINHLTMAYREGVVNIVKSRSFK | | | | |
| | GRQTREHLLTLLGQGKTVAGVSFDLGQKHAAGLLAAHFGL | | | | |
| | GEDGNPVFTPIQACFLPQRYLDSLTNYRNRYDALTLDMRR | | | | |
| | QSLLALTPAQQQEFADAQRDPGGQAKRACCLKLNLNPDEI | | | | |
| | RWDLVSGISTMISDLYIERGGDPRDVHQQVETKPKGKRKS | | | | |
| | EIRILKIRDGKWAYDFRPKIADETRKAQREQLWKLQKASS | | | | |
| | EFERLSRYKINIARAIANWALQWGRELSGCDIVIPVLEDL | | | | |
| | NVGSKFFDGKGKWLLGWDNRFTPKKENRWFIKVLHKAVAE | | | | |
| | LAPHRGVPVYEVMPHRTSMTCPACHYCHPTNREGDRFECQ | | | | |
| | SCHVVKNTDRDVAPYNILRVAVEGKTLDRWQAEKKPQAEP | | | | |
| | DRPMILIDNQES | | | | |
| CasΦ.11 | MSNKTTPPSPLSLLLRAHFPGLKFESQDYKIAGKKLRDGG | 11 | GUCUCAGCGUAC | 139 | NTTN |
| | PEAVISYLTGKGQAKLKDVKPPAKAFVIAQSRPFIEWDLV | | UGAGCAAUCAAA | | |
| | RVSRQIQEKIFGIPATKGRPKQDGLSETAFNEAVASLEVD | | AGGUUUCGCAGG | | |
| | GKSKLNEETRAAFYEVLGLDAPSLHAQAQNALIKSAISIR | | | | |
| | EGVLKKVENRNEKNLSKTKRRKEAGEEATFVEEKAHDERG | | | | |
| | YLIHPPGVNQTIPGYQAVVIKSCPSDFIGLPSGCLAKESA | | | | |
| | EALTDYLPHDRMTIPKGQPGYVPEWQHPLLNRRKNRRRRD | | | | |
| | WYSASLNKPKATCSKRSGTPNRKNSRTDQIQSGRFKGAIP | | | | |
| | VLMRFQDEWVIIDIRGLLRNARYRKLLKEKSTIPDLLSLF | | | | |
| | TGDPSIDMRQGVCTFIYKAGQACSAKMVKTKNAPEILSEL | | | | |
| | TKSGPVVLVSIDLGQTNPIAAKVSRVTQLSDGQLSHETLL | | | | |
| | RELLSNDSSDGKEIARYRVASDRLRDKLANLAVERLSPEH | | | | |
| | KSEILRAKNDTPALCKARVCAALGLNPEMIAWDKMTPYTE | | | | |
| | FLATAYLEKGGDRKVATLKPKNRPEMLRRDIKFKGTEGVR | | | | |
| | IEVSPEAAEAYREAQWDLQRISPEYLRLSTWKQELTKRIL | | | | |
| | NQLRHKAAKSSQCEVVVMAFEDLNIKMMHGNGKWADGGWD | | | | |
| | AFFIKKRENRWFMQAFHKSLTELGAHKGVPTIEVTPHRTS | | | | |
| | ITCTKCGHCDKANRDGERFACQKCGFVAHADLEIATDNIE | | | | |
| | RVALTGKPMPKPESERSGDAKKSVGARKAAFKPEEDAEAA | | | | |
| | E | | | | |
| CasΦ.12 | MIKPTVSQFLTPGFKLIRNHSRTAGLKLKNEGEEACKKFV | 12 | CUUUCAAGACUA | 140; | NTYN; NTTN; |
| | RENEIPKDECPNFQGGPAIANIIAKSREFTEWEIYQSSLA | | AUAGAUUGCUCC | 141 | TTTS |
| | IQEVIFTLPKDKLPEPILKEEWRAQWLSEHGLDTVPYKEA | | UUACGAGGAGAC; | | |
| | AGLNLIIKNAVNTYKGVQVKVDNKNKNNLAKINRKNEIAK | | AUUGCUCCUUAC | | |
| | LNGEQEISFEEIKAFDDKGYLLQKPSPNKSIYCYQSVSPK | | GAGGAGAC | | |
| | PFITSKYHNVNLPEEYIGYYRKSNEPIVSPYQFDRLRIPI | | | | |
| | GEPGYVPKWQYTFLSKKENKRRKLSKRIKNVSPILGIICI | | | | |
| | KKDWCVFDMRGLLRTNHWKKYHKPTDSINDLFDYFTGDPV | | | | |
| | IDTKANVVRFRYKMENGIVNYKPVREKKGKELLENICDQN | | | | |
| | GSCKLATVDVGQNNPVAIGLFELKKVNGELTKTLISRHPT | | | | |
| | PIDFCNKITAYRERYDKLESSIKLDAIKQLTSEQKIEVDN | | | | |
| | YNNNFTPQNTKQIVCSKLNINPNDLPWDKMISGTHFISEK | | | | |
| | AQVSNKSEIYFTSTDKGKTKDVMKSDYKWFQDYKPKLSKE | | | | |
| | VRDALSDIEWRLRRESLEFNKLSKSREQDARQLANWISSM | | | | |
| | CDVIGIENLVKKNNFFGGSGKREPGWDNFYKPKKENRWWI | | | | |
| | NAIHKALTELSQNKGKRVILLPAMRTSITCPKCKYCDSKN | | | | |
| | RNGEKFNCLKCGIELNADIDVATENLATVAITAQSMPKPT | | | | |
| | CERSGDAKKPVRARKAKAPEFHDKLAPSYTVVLREAV | | | | |
| CasΦ.13 | MRQPAEKTAFQVFRQEVIGTQKLSGGDAKTAGRLYKQGKM | 13 | GUAGAAGACCUC | 142 | |
| | EAAREWLLKGARDDVPPNFQPPAKCLVVAVSHPFEEWDIS | | GCUGAUUGCUCG | | |
| | KTNHDVQAYIYAQPLQAEGHLNGLSEKWEDTSADQHKLWF | | GUGCGCCGAGAC | | |
| | EKTGVPDRGLPVQAINKIAKAAVNRAFGVVRKVENRNEKR | | | | |
| | RSRDNRIAEHNRENGLTEVVREAPEVATNADGFLLHPPGI | | | | |
| | DPSILSYASVSPVPYNSSKHSFVRLPEEYQAYNVEPDAPI | | | | |
| | PQFVVEDRFAIPPGQPGYVPEWQRLKCSTNKHRRMRQWSN | | | | |
| | QDYKPKAGRRAKPLEFQAHLTRERAKGALLVVMRIKEDWV | | | | |
| | VFDVRGLLRNVEWRKVLSEEAREKLTLKGLLDLFTGDPVI | | | | |
| | DTKRGIVTFLYKAEITKILSKRTVKTKNARDLLLRLTEPG | | | | |
| | EDGLRREVGLVAVDLGQTHPIAAAIYRIGRTSAGALESTV | | | | |
| | LHRQGLREDQKEKLKEYRKRHTALDSRLRKEAFETLSVEQ | | | | |
| | QKEIVTVSGSGAQITKDKVCNYLGVDPSTLPWEKMGSYTH | | | | |
| | FISDDFLRRGGDPNIVHFDRQPKKGKVSKKSQRIKRSDSQ | | | | |
| | WVGRMRPRLSQETAKARMEADWAAQNENEEYKRLARSKQE | | | | |
| | LARWCVNTLLQNTRCITQCDEIVVVIEDLNVKSLHGKGAR | | | | |
| | EPGWDNFFTPKTENRWFIQILHKTFSELPKHRGEHVIEGC | | | | |
| | PLRTSITCPACSYCDKNSRNGEKFVCVACGATFHADFEVA | | | | |
| | TYNLVRLATTGMPMPKSLERQGGGEKAGGARKARKKAKQV | | | | |
| | EKIVVQANANVTMNGASLHSP | | | | |
| CasΦ.14 | MSSLPTPLELLKQKHADLFKGLQFSSKDNKMAGKVLKKDG | 14 | GGAUCCAAUCCU | 143 | |
| | EEAALAFLSERGVSRGELPNFRPPAKTLVVAQSRPFEEFP | | UUUUGAUUGCCC | | |
| | IYRVSEAIQLYVYSLSVKELETVPSGSSTKKEHQRFFQDS | | AAUUCGUUGGGA | | |
| | SVPDFGYTSVQGLNKIFGLARGIYLGVITRGENQLQKAKS | | C | | |
| | KHEALNKKRRASGEAETEFDPTPYEYMTPERKLAKPPGVN | | | | |
| | HSIMCYVDISVDEFDERNPDGIVLPSEYAGYCREINTAIE | | | | |
| | KGTVDRLGHLKGGPGYIPGHQRKESTTEGPKINFRKGRIR | | | | |
| | RSYTALYAKRDSRRVRQGKLALPSYRHHMMRLNSNAESAI | | | | |
| | LAVIFFGKDWVVFDLRGLLRNVRWRNLFVDGSTPSTLLGM | | | | |
| | FGDPVIDPKRGVVAFCYKEQIVPVVSKSITKMVKAPELLN | | | | |
| | KLYLKSEDPLVLVAIDLGQTNPVGVGVYRVMNASLDYEVV | | | | |
| | TRFALESELLREIESYRQRTNAFEAQIRAETFDAMTSEEQ | | | | |
| | EEITRVRAFSASKAKENVCHRFGMPVDAVDWATMGSNTIH | | | | |
| | IAKWVMRHGDPSLVEVLEYRKDNEIKLDKNGVPKKVKLTD | | | | |
| | KRIANLTSIRLRFSQETSKHYNDTMWELRRKHPVYQKLSK | | | | |
| | SKADESRRVVNSIIRRVNHLVPRARIVFIIEDLKNLGKVF | | | | |
| | HGSGKRELGWDSYFEPKSENRWFIQVLHKAFSETGKHKGY | | | | |
| | YIIECWPNWTSCTCPKCSCCDSENRHGEVERCLACGYTCN | | | | |
| | TDFGTAPDNLVKIATTGKGLPGPKKRCKGSSKGKNPKIAR | | | | |
| | SSETGVSVTESGAPKVKKSSPTQTSQSSSQSAP | | | | |
| CasΦ.15 | MIKPTVSQFLTPGFKLIRNHSRTAGLKLKNEGEEACKKFV | 15 | CUUUCAAGACUA | 144; | NTYN |
| | RENEIPKDECPNFQGGPAIANIIAKSREFTEWEIYQSSLA | | AUAGAUUGCUCC | 145 | |
| | IQEVIFTLPKDKLPEPILKEEWRAQWLSEHGLDTVPYKEA | | UUACGAGGAGAC; | | |
| | AGLNLIIKNAVNTYKGVQVKVDNKNKNNLAKINRKNEIAK | | AUUGCUCCUUAC | | |
| | LNGEQEISFEEIKAFDDKGYLLQKPSPNKSIYCYQSVSPK | | GAGGAGAC | | |
| | PFITSKYHNVNLPEEYIGYYRKSNEPIVSPYQFDRLRIPI | | | | |
| | GEPGYVPKWQYTFLSKKENKRRKLSKRIKNVSPILGIICI | | | | |
| | KKDWCVFDMRGLLRTNHWKKYHKPTDSINDLFDYFTGDPV | | | | |
| | IDTKANVVRFRYKMENGIVNYKPVREKKGKELLENICDQN | | | | |
| | GSCKLATVDVGQNNPVAIGLFELKKVNGELTKTLISRHPT | | | | |
| | PIDFCNKITAYRERYDKLESSIKLDAIKQLTSEQKIEVDN | | | | |
| | YNNNFTPQNTKQIVCSKLNINPNDLPWDKMISGTHFISEK | | | | |
| | AQVSNKSEIYFTSTDKGKTKDVMKSDYKWFQDYKPKLSKE | | | | |
| | VRDALSDIEWRLRRESLEFNKLSKSREQDARQLANWISSM | | | | |
| | CDVIGIENLVKKNNFFGGSGKREPGWDNFYKPKKENRWWI | | | | |
| | NAIHKALTELSQNKGKRVILLPAMRTSITCPKCKYCDSKN | | | | |
| | RNGEKFNCLKCGIELNADIDVATENLATVAITAQSMPKPT | | | | |
| | CERSGDAKKPVRARKAKAPEFHDKLAPSYTVVLREAV | | | | |
| CasΦ.16 | MSNKTTPPSPLSLLLRAHFPGLKFESQDYKIAGKKLRDGG | 16 | GUCUCAGCGUAC | 146 | NTTN |
| | PEAVISYLTGKGQAKLKDVKPPAKAFVIAQSRPFIEWDLV | | UGAGCAAUCAAA | | |
| | RVSRQIQEKIFGIPATKGRPKQDGLSETAFNEAVASLEVD | | AGGUUUCGCAGG | | |
| | GKSKLNEETRAAFYEVLGLDAPSLHAQAQNALIKSAISIR | | | | |
| | EGVLKKVENRNEKNLSKTKRRKEAGEEATFVEEKAHDERG | | | | |
| | YLIHPPGVNQTIPGYQAVVIKSCPSDFIGLPSGCLAKESA | | | | |
| | EALTDYLPHDRMTIPKGQPGYVPEWQHPLLNRRKNRRRRD | | | | |
| | WYSASLNKPKATCSKRSGTPNRKNSRTDQIQSGRFKGAIP | | | | |
| | VLMRFQDEWVIIDIRGLLRNARYRKLLKEKSTIPDLLSLF | | | | |
| | TGDPSIDMRQGVCTFIYKAGQACSAKMVKTKNAPEILSEL | | | | |
| | TKSGPVVLVSIDLGQTNPIAAKVSRVTQLSDGQLSHETLL | | | | |
| | RELLSNDSSDGKEIARYRVASDRLRDKLANLAVERLSPEH | | | | |
| | KSEILRAKNDTPALCKARVCAALGLNPEMIAWDKMTPYTE | | | | |
| | FLATAYLEKGGDRKVATLKPKNRPEMLRRDIKFKGTEGVR | | | | |
| | IEVSPEAAEAYREAQWDLQRTSPEYLRLSTWKQELTKRIL | | | | |
| | NQLRHKAAKSSQCEVVVMAFEDLNIKMMHGNGKWADGGWD | | | | |
| | AFFIKKRENRWFMQAFHKSLTELGAHKGVPTIEVTPHRTS | | | | |
| | ITCTKCGHCDKANRDGERFACQKCGFVAHADLEIATDNIE | | | | |
| | RVALTGKPMPKPESERSGDAKKSVGARKAAFKPEEDAEAA | | | | |
| | E | | | | |
| CasΦ.17 | MYSLEMADLKSEPSLLAKLLRDRFPGKYWLPKYWKLAEKK | 17 | AUGGCAACAGAC | 147 | |
| | RLTGGEEAACEYMADKQLDSPPPNFRPPARCVILAKSRPF | | UCUCAUUGCGCG | | |
| | EDWPVHRVASKAQSFVIGLSEQGFAALRAAPPSTADARRD | | GUACGCCGCGAC | | |
| | WLRSHGASEDDLMALEAQLLETIMGNAISLHGGVLKKIDN | | | | |
| | ANVKAAKRLSGRNEARLNKGLQELPPEQEGSAYGADGLLV | | | | |
| | NPPGLNLNIYCRKSCCPKPVKNTARFVGHYPGYLRDSDSI | | | | |
| | LISGTMDRLTIIEGMPGHIPAWQREQGLVKPGGRRRRLSG | | | | |
| | SESNMRQKVDPSTGPRRSTRSGTVNRSNQRTGRNGDPLLV | | | | |
| | EIRMKEDWVLLDARGLLRNLRWRESKRGLSCDHEDLSLSG | | | | |
| | LLALFSGDPVIDPVRNEVVFLYGEGIIPVRSTKPVGTRQS | | | | |
| | KKLLERQASMGPLTLISCDLGQTNLIAGRASAISLTHGSL | | | | |
| | GVRSSVRIELDPEIIKSFERLRKDADRLETEILTAAKETL | | | | |
| | SDEQRGEVNSHEKDSPQTAKASLCRELGLHPPSLPWGQMG | | | | |
| | PSTTFIADMLISHGRDDDAFLSHGEFPTLEKRKKFDKRFC | | | | |
| | LESRPLLSSETRKALNESLWEVKRTSSEYARLSQRKKEMA | | | | |
| | RRAVNFVVEISRRKTGLSNVIVNIEDLNVRIFHGGGKQAP | | | | |
| | GWDGFFRPKSENRWFIQAIHKAFSDLAAHHGIPVIESDPQ | | | | |
| | RTSMTCPECGHCDSKNRNGVRFLCKGCGASMDADFDAACR | | | | |
| | NLERVALTGKPMPKPSTSCERLLSATTGKVCSDHSLSHDA | | | | |
| | IEKAS | | | | |
| CasΦ.18 | MEKEITELTKIRREFPNKKFSSTDMKKAGKLLKAEGPDAV | 18 | ACCAAAACGACU | 148 | VTTN |
| | RDFLNSCQEIIGDFKPPVKTNIVSISRPFEEWPVSMVGRA | | AUUGAUUGCCCA | | |
| | IQEYYFSLTKEELESVHPGTSSEDHKSFFNITGLSNYNYT | | GUACGCUGGGAC | | |
| | SVQGLNLIFKNAKAIYDGTLVKANNKNKKLEKKFNEINHK | | | | |
| | RSLEGLPIITPDFEEPFDENGHLNNPPGINRNIYGYQGCA | | | | |
| | AKVFVPSKHKMVSLPKEYEGYNRDPNLSLAGERNRLEIPE | | | | |
| | GEPGHVPWFQRMDIPEGQIGHVNKIQRFNFVHGKNSGKVK | | | | |
| | FSDKTGRVKRYHHSKYKDATKPYKFLEESKKVSALDSILA | | | | |
| | IITIGDDWVVFDIRGLYRNVFYRELAQKGLTAVQLLDLFT | | | | |
| | GDPVIDPKKGVVTFSYKEGVVPVFSQKIVPRFKSRDTLEK | | | | |
| | LTSQGPVALLSVDLGQNEPVAARVCSLKNINDKITLDNSC | | | | |
| | RISFLDDYKKQIKDYRDSLDELEIKIRLEAINSLETNQQV | | | | |
| | EIRDLDVFSADRAKANTVDMFDIDPNLISWDSMSDARVST | | | | |
| | QISDLYLKNGGDESRVYFEINNKRIKRSDYNISQLVRPKL | | | | |
| | SDSTRKNLNDSIWKLKRISEEYLKLSKRKLELSRAVVNYT | | | | |
| | IRQSKLLSGINDIVIILEDLDVKKKENGRGIRDIGWDNFF | | | | |
| | SSRKENRWFIPAFHKTFSELSSNRGLCVIEVNPAWTSATC | | | | |
| | PDCGFCSKENRDGINFTCRKCGVSYHADIDVATLNIARVA | | | | |
| | VLGKPMSGPADRERLGDTKKPRVARSRKTMKRKDISNSTV | | | | |
| | EAMVTA | | | | |
| CasΦ.19 | MLVRTSTLVQDNKNSRSASRAFLKKPKMPKNKHIKEPTEL | 19 | GUCGCUCUCUAA | 149 | |
| | AKLIRELFPGQRFTRAINTQAGKILKHKGRDEVVEFLKNK | | CGCUUGCCCAGU | | |
| | GIDKEQFMDFRPPTKARIVATSGAIEEFSYLRVSMAIQEC | | ACGCUGGGAC | | |
| | CFGKYKFPKEKVNGKLVLETVGLTKEELDDFLPKKYYENK | | | | |
| | KSRDRFFLKTGICDYGYTYAQGLNEIFRNTRAIYEGVFTK | | | | |
| | VNNRNEKRREKKDKYNEERRSKGLSEEPYDEDESATDESG | | | | |
| | HLINPPGVNLNIWTCEGFCKGPYVTKLSGTPGYEVILPKV | | | | |
| | FDGYNRDPNEIISCGITDRFAIPEGEPGHIPWHQRLEIPE | | | | |
| | GQPGYVPGHQRFADTGQNNSGKANPNKKGRMRKYYGHGTK | | | | |
| | YTQPGEYQEVFRKGHREGNKRRYWEEDFRSEAHDCILYVI | | | | |
| | HIGDDWVVCDLRGPLRDAYRRGLVPKEGITTQELCNLFSG | | | | |
| | DPVIDPKHGVVTFCYKNGLVRAQKTISAGKKSRELLGALT | | | | |
| | SQGPIALIGVDLGQTEPVGARAFIVNQARGSLSLPTLKGS | | | | |
| | FLLTAENSSSWNVFKGEIKAYREAIDDLAIRLKKEAVATL | | | | |
| | SVEQQTEIESYEAFSAEDAKQLACEKFGVDSSFILWEDMT | | | | |
| | PYHTGPATYYFAKQFLKKNGGNKSLIEYIPYQKKKSKKTP | | | | |
| | KAVLRSDYNIACCVRPKLLPETRKALNEAIRIVQKNSDEY | | | | |
| | QRLSKRKLEFCRRVVNYLVRKAKKLTGLERVIIAIEDLKS | | | | |
| | LEKFFTGSGKRDNGWSNFFRPKKENRWFIPAFHKAFSELA | | | | |
| | PNRGFYVIECNPARTSITDPDCGYCDGDNRDGIKFECKKC | | | | |
| | GAKHHTDLDVAPLNIAIVAVTGRPMPKTVSNKSKRERSGG | | | | |
| | EKSVGASRKRNHRKSKANQEMLDATSSAAE | | | | |
| CasΦ.20 | MPKIKKPTEISLLRKEVFPDLHFAKDRMRAASLVLKNEGR | 20 | | | NTTN; NTNN |
| | EAAIEYLRVNHEDKPPNFMPPAKTPYVALSRPLEQWPIAQ | | | | |
| | ASIAIQKYIFGLTKDEFSATKKLLYGDKSTPNTESRKRWF | | | | |
| | EVTGVPNFGYMSAQGLNAIFSGALARYEGVVQKVENRNKK | | | | |
| | RFEKLSEKNQLLIEEGQPVKDYVPDTAYHTPETLQKLAEN | | | | |
| | NHVRVEDLGDMIDRLVHPPGIHRSIYGYQQVPPFAYDPDN | | | | |
| | PKGIILPKAYAGYTRKPHDIIEAMPNRLNIPEGQAGYIPE | | | | |
| | HQRDKLKKGGRVKRLRTTRVRVDATETVRAKAEALNAEKA | | | | |
| | RLRGKEAILAVFQIEEDWALIDMRGLLRNVYMRKLIAAGE | | | | |
| | LTPTTLLGYFTETLTLDPRRTEATFCYHLRSEGALHAEYV | | | | |
| | RHGKNTRELLLDLTKDNEKIALVTIDLGQRNPLAAAIFRV | | | | |
| | GRDASGDLTENSLEPVSRMLLPQAYLDQIKAYRDAYDSFR | | | | |
| | QNIWDTALASLTPEQQRQILAYEAYTPDDSKENVLRLLLG | | | | |
| | GNVMPDDLPWEDMTKNTHYISDRYLADGGDPSKVWFVPGP | | | | |
| | RKRKKNAPPLKKPPKPRELVKRSDHNISHLSEFRPQLLKE | | | | |
| | TRDAFEKAKIDTERGHVGYQKLSTRKDQLCKEILNWLEAE | | | | |
| | AVRLTRCKTMVLGLEDLNGPFFNQGKGKVRGWVSFFRQKQ | | | | |
| | ENRWIVNGFRKNALARAHDKGKYILELWPSWTSQTCPKCK | | | | |
| | HVHADNRHGDDFVCLQCGARLHADAEVATWNLAVVAIQGH | | | | |
| | SLPGPVREKSNDRKKSGSARKSKKANESGKVVGAWAAQAT | | | | |
| | PKRATSKKETGTARNPVYNPLETQASCPAP | | | | |
| CasΦ.21 | MTPSPQIARLVETPLAAALKAHHPGKKFRSDYLKKAGKIL | 21 | GGUUGAACCCUC | 150 | NTTG |
| | KDQGVEAAMAHLDGKDQAEPPNFKPPAKCRIVARSREFSE | | AACAGAUUGCUC | | |
| | WPIVKASVEIQKYIYGLTLEERKACDPGKSSASHKAWFAK | | GGUAAGCCGAGA | | |
| | TGVNTFGYSSVQGFNLIFGHTLGRYDGVLVKTENLNKKRA | | C | | |
| | EKNERFRAKALAEGRAEPVCPPLVTAINDTGQDVTLEDGR | | | | |
| | VVRPGQLLQPPGINPNIYAYQQVSPKAYVPGIIELPEEFQ | | | | |
| | GYSRDPNAVILPLVPRDRLSIPKGQPGYVPEPHREGLIGR | | | | |
| | KDRRMRRYYETERGTKLKRPPLTAKGRADKANEALLVVVR | | | | |
| | IDSDWVVMDVRGLLRNARWRRLVSKEGITLNGLLDLFTGD | | | | |
| | PVLNPKDCSVSRDTGDPVNDPRHGVVTFCYKLGVVDVCSK | | | | |
| | DRPIKGFRTKEVLERLTSSGTVGMVSIDLGQTNPVAAAVS | | | | |
| | RVTKGLQAETLETFTLPDDLLGKVRAYRAKTDRMEEGFRR | | | | |
| | NALRKLTAEQQAEITRYNDATEQQAKALVCSTYGIGPEEV | | | | |
| | PWERMTSNTTYISDHILDHGGDPDTVFFMATKRGQNKPTL | | | | |
| | HKRKDKAWGQKFRPAISVETRLARQAAEWELRRASLEFQK | | | | |
| | LSVWKTELCRQAVNYVMERTKKRTQCDVIIPVIEDLPVPL | | | | |
| | FHGSGKRDPGWANFFVHKRENRWFIDGLHKAFSELGKHRG | | | | |
| | IYVFEVCPQRTSITCPKCGHCDPDNRDGEKFVCLSCQATL | | | | |
| | NADLDVATTNLVRVALTGKVMPRSERSGDAQTPGPARKAR | | | | |
| | TGKIKGSKPTSAPQGATQTDAKAHLSQTGV | | | | |
| CasΦ.22 | MTPSPQIARLVETPLAAALKAHHPGKKFRSDYLKKAGKIL | 22 | GGUUGAACCCUC | 151 | |
| | KDQGVEAAMAHLDGKDQAEPPNFKPPAKCRIVARSREFSE | | AACAGAUUGCUC | | |
| | WPIVKASVEIQKYIYGLTLEERKACDPGKSSASHKAWFAK | | GGUAAGCCGAGA | | |
| | TGVNTFGYSSVQGFNLIFGHTLGRYDGVLVKTENLNKKRA | | C | | |
| | EKNERFRAKALAEGRAEPVCPPLVTATNDTGQDVTLEDGR | | | | |
| | VVRPGQLLQPPGINPNIYAYQQVSPKAYVPGIIELPEEFQ | | | | |
| | GYSRDPNAVILPLVPRDRLSIPKGQPGYVPEPHREGLTGR | | | | |
| | KDRRMRRYYETERGTKLKRPPLTAKGRADKANEALLVVVR | | | | |
| | IDSDWVVMDVRGLLRNARWRRLVSKEGITLNGLLDLFTGD | | | | |
| | PVLNPKDCSVSRDTGDPVNDPRHGVVTFCYKLGVVDVCSK | | | | |
| | DRPIKGFRTKEVLERLTSSGTVGMVSIDLGQTNPVAAAVS | | | | |
| | RVTKGLQAETLETFTLPDDLLGKVRAYRAKTDRMEEGFRR | | | | |
| | NALRKLTAEQQAEITRYNDATEQQAKALVCSTYGIGPEEV | | | | |
| | PWERMTSNTTYISDHILDHGGDPDTVFFMATKRGQNKPTL | | | | |
| | HKRKDKAWGQKFRPAISVETRLARQAAEWELRRASLEFQK | | | | |
| | LSVWKTELCRQAVNYVMERTKKRTQCDVIIPVIEDLPVPL | | | | |
| | FHGSGKRDPGWANFFVHKRENRWFIDGLHKAFSELGKHRG | | | | |
| | IYVFEVCPQRTSITCPKCGHCDPDNRDGEKFVCLSCQATL | | | | |
| | HADLDVATTNLVRVALTGKVMPRSERSGDAQTPGPARKAR | | | | |
| | TGKIKGSKPTSAPQGATQTDAKAHLSQTGV | | | | |
| Caso.23 | MKTEKPKTALTLLREEVFPGKKYRLDVLKEAGKKLSTKGR | 23 | CUUGAAAUCCUG | 152 | |
| | EATIEFLTGKDEERPQNFQPPAKTSIVAQSRPFDQWPIVQ | | UCAGAUUGCUCC | | |
| | VSLAVQKYIYGLTQSEFEANKKALYGETGKAISTESRRAW | | CUUCGGGGAGAC | | |
| | FEATGVDNFGFTAAQGINPIFSQAVARYEGVIKKVENRNE | | | | |
| | KKLKKLTKKNLLRLESGEEIEDFEPEATFNEEGRLLQPPG | | | | |
| | ANPNIYCYQQISPRIYDPSDPKGVILPQIYAGYDRKPEDI | | | | |
| | ISAGVPNRLAIPEGQPGYIPEHQRAGLKTQGRIRCRASVE | | | | |
| | AKARAAILAVVHLGEDWVVLDLRGLLRNVYWRKLASPGTL | | | | |
| | TLKGLLDFFTGGPVLDARRGIATFSYTLKSAAAVHAENTY | | | | |
| | KGKGTREVLLKLTENNSVALVTVDLGQRNPLAAMIARVSR | | | | |
| | TSQGDLTYPESVEPLTRLFLPDPFLEEVRKYRSSYDALRL | | | | |
| | SIREAAIASLTPEQQAEIRYIEKFSAGDAKKNVAEVFGID | | | | |
| | PTQLPWDAMTPRTTYISDLFLRMGGDRSRVFFEVPPKKAK | | | | |
| | KAPKKPPKKPAGPRIVKRTDGMIARLREIRPRLSAETNKA | | | | |
| | FQEARWEGERSNVAFQKLSVRRKQFARTVVNHLVQTAQKM | | | | |
| | SRCDTVVLGIEDLNVPFFHGRGKYQPGWEGFFRQKKENRW | | | | |
| | LINDMHKALSERGPHRGGYVLELTPFWTSLRCPKCGHTDS | | | | |
| | ANRDGDDFVCVKCGAKLHSDLEVATANLALVAITGQSIPR | | | | |
| | PPREQSSGKKSTGTARMKKTSGETQGKGSKACVSEALNKI | | | | |
| | EQGTARDPVYNPLNSQVSCPAP | | | | |
| CasΦ.24 | VYNPDMKKPNNIRRIREEHFEGLCFGKDVLTKAGKIYEKD | 24 | GCUGGAAGACUC | 153 | NTYN |
| | GEEAAIDFLMGKDEEDPPNFKPPAKTTIVAQSRPFDQWPI | | AAUGAUGGCUCC | | |
| | YQVSQAVQERVFAYTEEEFNASKEALFSGDISSKSRDFWF | | UUACGAGGAGAC | | |
| | KINNISDQGIGAQGLNTILSHAFSRYSGVIKKVENRNKKR | | | | |
| | LKKLSKKNQLKIEEGLEILEFKPDSAFNENGLLAQPPGIN | | | | |
| | PNIYGYQAVTPFVFDPDNPGDVILPKQYEGYSRKPDDIIE | | | | |
| | KGPSRLDIPKGQPGYVPEHQRKNLKKKGRVRLYRRTPPKT | | | | |
| | KALASILAVLQIGKDWVLFDMRGLLRSVYMREAATPGQIS | | | | |
| | AKDLLDTFTGCPVLNTRTGEFTFCYKLRSEGALHARKIYT | | | | |
| | KGETRTLLTSLISENNTIALVTVDLGQRNPAAIMISRLSR | | | | |
| | KEELSEKDIQPVSRRLLPDRYLNELKRYRDAYDAFRQEVR | | | | |
| | DEAFTSLCPEHQEQVQQYEALTPEKAKNLVLKHFFGTHDP | | | | |
| | DLPWDDMTSNTHYIANLYLERGGDPSKVFFTRPLKKDSKS | | | | |
| | KKPRKPTKRTDASISRLPEIRPKMPEDARKAFEKAKWEIY | | | | |
| | TGHEKFPKLAKRVNQLCREIANWIEKEAKRLTLCDTVVVG | | | | |
| | IEDLSLPPKRGKGKFQETWQGFFRQKFENRWVIDTLKKAI | | | | |
| | QNRAHDKGKYVLGLAPYWTSQRCPACGFIHKSNRNGDHFK | | | | |
| | CLKCEALFHADSEVATWNLALVAVLGKGITNPDSKKPSGQ | | | | |
| | KKTGTTRKKQIKGKNKGKETVNVPPTTQEVEDIIAFFEKD | | | | |
| | DETVRNPVYKPTGT | | | | |
| CasΦ.25 | MKKPNNIRRIREEHFEGLCFGKDVLTKAGKIYEKDGEEAA | 25 | GCUGGAAGACUC | 154 | NTYN |
| | IDFLMGKDEEDPPNFKPPAKTTIVAQSRPFDQWPIYQVSQ | | AAUGAUGGCUCC | | |
| | AVQERVFAYTEEEFNASKEALFSGDISSKSRDFWFKINNI | | UUACGAGGAGAC | | |
| | SDQGIGAQGLNTILSHAFSRYSGVIKKVENRNKKRLKKLS | | | | |
| | KKNQLKIEEGLEILEFKPDSAFNENGLLAQPPGINPNIYG | | | | |
| | YQAVTPFVFDPDNPGDVILPKQYEGYSRKPDDIIEKGPSR | | | | |
| | LDIPKGQPGYVPEHQRKNLKKKGRVRLYRRTPPKTKALAS | | | | |
| | ILAVLQIGKDWVLFDMRGLLRSVYMREAATPGQISAKDLL | | | | |
| | DTFTGCPVLNTRTGEFTFCYKLRSEGALHARKIYTKGETR | | | | |
| | TLLTSLTSENNTIALVTVDLGQRNPAAIMISRLSRKEELS | | | | |
| | EKDIQPVSRRLLPDRYLNELKRYRDAYDAFRQEVRDEAFT | | | | |
| | SLCPEHQEQVQQYEALTPEKAKNLVLKHFFGTHDPDLPWD | | | | |
| | DMTSNTHYIANLYLERGGDPSKVFFTRPLKKDSKSKKPRK | | | | |
| | PTKRTDASISRLPEIRPKMPEDARKAFEKAKWEIYTGHEK | | | | |
| | FPKLAKRVNQLCREIANWIEKEAKRLTLCDTVVVGIEDLS | | | | |
| | LPPKRGKGKFQETWQGFFRQKFENRWVIDTLKKAIQNRAH | | | | |
| | DKGKYVLGLAPYWTSQRCPACGFIHKSNRNGDHFKCLKCE | | | | |
| | ALFHADSEVATWNLALVAVLGKGITNPDSKKPSGQKKTGT | | | | |
| | TRKKQIKGKNKGKETVNVPPTTQEVEDIIAFFEKDDETVR | | | | |
| | NPVYKPTGT | | | | |
| CasΦ.26 | VIKTHFPAGRFRKDHQKTAGKKLKHEGEEACVEYLRNKVS | 26 | CUAGGAACGCAC | 155 | NTTN |
| | DYPPNFKPPAKGTIVAQSRPFSEWPIVRASEAIQKYVYGL | | GCAGAUUGCUCG | | |
| | TVAELDVFSPGTSKPSHAEWFAKTGVENYGYRQVQGLNTI | | GUACGCCGAGAC | | |
| | FQNTVNRFKGVLKKVENRNKKSLKRQEGANRRRVEEGLPE | | | | |
| | VPVTVESATDDEGRLLQPPGVNPSIYGYQGVAPRVCTDLQ | | | | |
| | GFSGMSVDFAGYRRDPDAVLVESLPEGRLSIPKGERGYVP | | | | |
| | EWQRDPERNKFPLREGSRRQRKWYSNACHKPKPGRTSKYD | | | | |
| | PEALKKASAKDALLVSISIGEDWAIIDVRGLLRDARRRGF | | | | |
| | TPEEGLSLNSLLGLFTEYPVFDVQRGLITFTYKLGQVDVH | | | | |
| | SRKTVPTFRSRALLESLVAKEEIALVSVDLGQTNPASMKV | | | | |
| | SRVRAQEGALVAEPVHRMFLSDVLLGELSSYRKRMDAFED | | | | |
| | AIRAQAFETMTPEQQAEITRVCDVSVEVARRRVCEKYSIS | | | | |
| | PQDVPWGEMTGHSTFIVDAVLRKGGDESLVYFKNKEGETL | | | | |
| | KFRDLRISRMEGVRPRLTKDTRDALNKAVLDLKRAHPTFA | | | | |
| | KLAKQKLELARRCVNFIEREAKRYTQCERVVFVIEDLNVG | | | | |
| | FFHGKGKRDRGWDAFFTAKKENRWVIQALHKAFSDLGLHR | | | | |
| | GSYVIEVTPQRTSMTCPRCGHCDKGNRNGEKFVCLQCGAT | | | | |
| | LHADLEVATDNIERVALTGKAMPKPPVRERSGDVQKAGTA | | | | |
| | RKARKPLKPKQKTEPSVQEGSSDDGVDKSPGDASRNPVYN | | | | |
| | PSDTLSI | | | | |
| CasΦ.27 | MAKAKTLAALLRELLPGQHLAPHHRWVANKLLMTSGDAAA | 27 | AUUGCAACGCCU | 156 | RTTS |
| | FVIGKSVSDPVRGSFRKDVITKAGRIFKKDGPDAAAAFLD | | AAAGAUUGCUCG | | |
| | GKWEDRPPNFQPPAKAAIVAISRSFDEWPIVKVSCAIQQY | | AUACGUCGAGAC | | |
| | LYALPVQEFESSVPEARAQAHAAWFQDTGVDDCNFKSTQG | | | | |
| | LNAIFNHGKRTYEGVLKKAQNRNDKKNLRLERINAKRAEA | | | | |
| | GQAPLVAGPDESPTDDAGCLLHPPGINANIYCYQQVSPRP | | | | |
| | YEQSCGIQLPPEYAGYNRLSNVAIPPMPNRLDIPQGQPGY | | | | |
| | VPEHHRHGIKKFGRVRKRYGVVPGRNRDADGKRTRQVLTE | | | | |
| | AGAAAKARDSVLAVIRIGDDWTVVDLRGLLRNAQWRKLVP | | | | |
| | DGGITVQGLLDLFTGDPVIDPRRGVVTFIYKADSVGIHSE | | | | |
| | KVCRGKQSKNLLERLCAMPEKSSTRLDCARQAVALVSVDL | | | | |
| | GQRNPVAARFSRVSLAEGQLQAQLVSAQFLDDAMVAMIRS | | | | |
| | YREEYDRFESLVREQAKAALSPEQLSEIVRHEADSAESVK | | | | |
| | SCVCAKFGIDPAGLSWDKMTSGTWRIADHVQAAGGDVEWE | | | | |
| | FFKTCGKGKEIKTVRRSDFNVAKQFRLRLSPETRKDWNDA | | | | |
| | IWELKRGNPAYVSFSKRKSEFARRVVNDLVHRARRAVRCD | | | | |
| | EVVFAIEDLNISFFHGKGQRQMGWDAFFEVKQENRWFIQA | | | | |
| | LHKAFVERATHKGGYVLEVAPARTSTTCPECRHCDPESRR | | | | |
| | GEQFCCIKCRHTCHADLEVATFNIEQVALTGVSLPKRLSS | | | | |
| | TLL | | | | |
| CasΦ.28 | MSKEKTPPSAYAILKAKHFPDLDFEKKHKMMAGRMFKNGA | 28 | GUUCGGCRAYCC | 157 | NTTN; NTTB |
| | SEQEVVQYLQGKGSESLMDVKPPAKSPILAQSRPFDEWEM | | UUUGAUUGCUCA | | |
| | VRTSRLIQETIFGIPKRGSIPKRDGLSETQFNELVASLEV | | GUACGCUGAGAC | | |
| | GGKPMLNKQTRAIFYGLLGIKPPTFHAMAQNILIDLAINI | | | | |
| | RKGVLKKVDNLNEKNRKKVKRIRDAGEQDVMVPAEVTAHD | | | | |
| | DRGYLNHPPGVNPTIPGYQGVVIPFPEGFEGLPSGMTPVD | | | | |
| | WSHVLVDYLPHDRLSIPKGSPGYIPEWQRPLLNRHKGRRH | | | | |
| | RSWYANSLNKPRKSRTEEAKDRQNAGKRTALIEAERLKGV | | | | |
| | LPVLMRFKEDWLIIDARGLLRNARYRGVLPEGSTLGNLID | | | | |
| | LFSDSPRVDTRRGICTFLYRKGRAYSTKPVKRKESKETLL | | | | |
| | KLTEKSTIALVSIDLGQTNPLTAKLSKVRQVDGCLVAEPV | | | | |
| | LRKLIDNASEDGKEIARYRVAHDLLRARILEDAIDLLGIY | | | | |
| | KDEVVRARSDTPDLCKERVCRFLGLDSQAIDWDRMTPYTD | | | | |
| | FIAQAFVAKGGDPKVVTIKPNGKPKMFRKDRSIKNMKGIR | | | | |
| | LDISKEASSAYREAQWAIQRESPDFQRLAVWQSQLTKRIV | | | | |
| | NQLVAWAKKCTQCDTVVLAFEDLNIGMMHGSGKWANGGWN | | | | |
| | ALFLHKQENRWFMQAFHKALTELSAHKGIPTIEVLPHRTS | | | | |
| | ITCTQCGHCHPGNRDGERFKCLKCEFLANTDLEIATDNIE | | | | |
| | RVALTGLPMPKGERSSAKRKPGGTRKTKKSKHSGNSPLAA | | | | |
| | E | | | | |
| CasΦ.29 | MEKAGPTSPLSVLIHKNFEGCRFQIDHLKIAGRKLAREGE | 29 | GUUGAACCUAGA | 158 | NTTN |
| | AAAIEYLLDKKCEGLPPNFQPPAKGNVIAQSRPFTEWAPY | | UCAGAUGGCUCA | | |
| | RASVAIQKYIYSLSVDERKVCDPGSSSDSHEKWFKQTGVQ | | GUACGCUGAGAC | | |
| | NYGYTHVQGLNLIFKHALARYDGVLKKVDNRNEKNRKKAE | | | | |
| | RVNSFRREEGLPEEVFEEEKATDETGHLLQPPGVNHSIYC | | | | |
| | YQSVRPKPFNPRKPGGISLPEAYSGYSLKPQDELPIGSLD | | | | |
| | RLSIPPGQPGYVPEWQRSQLTTQKHRRKRSWYSAQKWKPR | | | | |
| | TGRTSTFDPDRLNCARAQGAILAVVRIHEDWVVFDVRGLL | | | | |
| | RNALWRELAGKGLTVRDLLDFFTGDPVVDTKRGVVTFTYK | | | | |
| | LGKVDVHSLRTVRGKRSKKVLEDLTLSSDVGLVTIDLGQT | | | | |
| | NVLAADYSKVTRSENGELLAVPLSKSFLPKHLLHEVTAYR | | | | |
| | TSYDQMEEGFRRKALLTLTEDQQVEVTLVRDFSVESSKTK | | | | |
| | LLQLGVDVTSLPWEKMSSNTTYISDQLLQQGADPASLFFD | | | | |
| | GERDGKPCRHKKKDRTWAYLVRPKVSPETRKALNEALWAL | | | | |
| | KNTSPEFESLSKRKIQFSRRCMNYLLNEAKRISGCGQVVF | | | | |
| | VIEDLNVRVHHGRGKRAIGWDNFFKPKRENRWFMQALHKA | | | | |
| | ASELAIHRGMHIIEACPARSSITCPKCGHCDPENRCSSDR | | | | |
| | EKFLCVKCGAAFHADLEVATENLRKVALTGTALPKSIDHS | | | | |
| | RDGLIPKGARNRKLKEPQANDEKACA | | | | |
| CasΦ.30 | MKEQSPLSSVLKSNFPGKKFLSADIRVAGRKLAQLGEAAA | 30 | CCCUCAACACGU | 159 | VTTN |
| | VEYLSPRQRDSVPNFRPPAFCTVVAKSRPFEEWPIYKASV | | CAGAAAUGCCCG | | |
| | LLQEQIYGMTGQEFEERCGSIPTSLSGLRQWASSVGLGAA | | GCACGCCGGGAC | | |
| | MEGLHVQGMNLMVKNAINRYKGVLVKVENRNKKLVEANEA | | | | |
| | KNSSREERGLPPLRPPELGSAFGPDGRLVNPPGIDKSIRL | | | | |
| | YQGVSPVPVVKTTGRPTVHRLDIPAGEKGHVPLWQREAGL | | | | |
| | VKEGPRRRRMWYSNSNLKRSRKDRSAEASEARKADSVVVR | | | | |
| | VSVKEDWVDIDVRGLLRNVAWRGIERAGESTEDLLSLFSG | | | | |
| | DPVVDPSRDSVVFLYKEGVVDVLSKKVVGAGKSRKQLEKM | | | | |
| | VSEGPVALVSCDLGQTNYVAARVSVLDESLSPVRSFRVDP | | | | |
| | REFPSADGSQGVVGSLDRIRADSDRLEAKLLSEAEASLPE | | | | |
| | PVRAEIEFLRSERPSAVAGRLCLKLGIDPRSIPWEKMGST | | | | |
| | TSFISEALSAKGSPLALHDGAPIKDSRFAHAARGRLSPES | | | | |
| | RKALNEALWERKSSSREYGVISRRKSEASRRMANAVLSES | | | | |
| | RRLTGLAVVAVNLEDLNMVSKFFHGRGKRAPGWAGFFTPK | | | | |
| | MENRWFIRSIHKAMCDLSKHRGITVIESRPERTSISCPEC | | | | |
| | GHCDPENRSGERFSCKSCGVSLHADFEVATRNLERVALTG | | | | |
| | KPMPRRENLHSPEGATASRKTRKKPREATASTFLDLRSVL | | | | |
| | SSAENEGSGPAARAG | | | | |
| CasΦ.31 | MLPPSNKIGKSMSLKEFINKRNFKSSIIKQAGKILKKEGE | 31 | GUCGCAAGACUC | 160 | NTTN |
| | EAVKKYLDDNYVEGYKKRDFPITAKCNIVASNRKIEDFDI | | GAAUAAUUGCCC | | |
| | SKFSSFIQNYVFNLNKDNFEEFSKIKYNRKSFDELYKKIA | | CUCUAUGGGGAC | | |
| | NEIGLEKPNYENIQGEIAVIRNAINIYNGVLKKVENRNKK | | | | |
| | IQEKNQSKDPPKLLSAFDDNGFLAERPGINETIYGYQSVR | | | | |
| | LRHLDVEKDKDIIVQLPDIYQKYNKKSTDKISVKKRLNKY | | | | |
| | NVDEYGKLISKRRKERINKDDAILCVSNFGDDWIIFDARG | | | | |
| | LLRQTYRYKLKKKGLCIKDLLNLFTGDPIINPTKTDLKEA | | | | |
| | LSLSFKDGIINNRTLKVKNYKKCPELISELIRDKGKVAMI | | | | |
| | SIDLGQTNPISYRLSKFTANNVAYIENGVISEDDIVKMKK | | | | |
| | WREKSDKLENLIKEEAIASLSDDEQREVRLYENDIADNTK | | | | |
| | KKILEKFNIREEDLDFSKMSNNTYFIRDCLKNKNIDESEF | | | | |
| | TFEKNGKKLDPTDACFAREYKNKLSELTRKKINEKIWEIK | | | | |
| | KNSKEYHKISIYKKETIRYIVNKLIKQSKEKSECDDIIVN | | | | |
| | IEKLQIGGNFFGGRGKRDPGWNNFFLPKEENRWFINACHK | | | | |
| | AFSELAPHKGIIVIESDPAYTSQTCPKCENCDKENRNGEK | | | | |
| | FKCKKCNYEANADIDVATENLEKIAKNGRRLIKNFDQLGE | | | | |
| | RLPGAEMPGGARKRKPSKSLPKNGRGAGVGSEPELINQSP | | | | |
| | SQVIA | | | | |
| CasΦ.32 | VPDKKETPLVALCKKSFPGLRFKKHDSRQAGRILKSKGEG | 32 | GCUGGGGACCGA | 161 | NTYN |
| | AAVAFLEGKGGTTQPNFKPPVKCNIVAMSRPLEEWPIYKA | | UCCUGAUUGCUC | | |
| | SVVIQKYVYAQSYEEFKATDPGKSEAGLRAWLKATRVDTD | | GCUGCGGCGAGA | | |
| | GYFNVQGLNLIFQNARATYEGVLKKVENRNSKKVAKIEQR | | C | | |
| | NEHRAERGLPLLTLDEPETALDETGHLRHRPGINCSVFGY | | | | |
| | QHMKLKPYVPGSIPGVTGYSRDPSTPIAACGVDRLEIPEG | | | | |
| | QPGYVPPWDRENLSVKKHRRKRASWARSRGGAIDDNMLLA | | | | |
| | VVRVADDWALLDLRGLLRNTQYRKLLDRSVPVTIESLLNL | | | | |
| | VTNDPTLSVVKKPGKPVRYTATLIYKQGVVPVVKAKVVKG | | | | |
| | SYVSKMLDDTTETFSLVGVDLGVNNLIAANALRIRPGKCV | | | | |
| | ERLQAFTLPEQTVEDFFRFRKAYDKHQENLRLAAVRSLTA | | | | |
| | EQQAEVLALDTFGPEQAKMQVCGHLGLSVDEVPWDKVNSR | | | | |
| | SSILSDLAKERGVDDTLYMFPFFKGKGKKRKTEIRKRWDV | | | | |
| | NWAQHFRPQLTSETRKALNEAKWEAERNSSKYHQLSIRKK | | | | |
| | ELSRHCVNYVIRTAEKRAQCGKVIVAVEDLHHSFRRGGKG | | | | |
| | SRKSGWGGFFAAKQEGRWLMDALFGAFCDLAVHRGYRVIK | | | | |
| | VDPYNTSRTCPECGHCDKANRDRVNREAFICVCCGYRGNA | | | | |
| | DIDVAAYNIAMVAITGVSLRKAARASVASTPLESLAAE | | | | |
| CasΦ.33 | MSKTKELNDYQEALARRLPGVRHQKSVRRAARLVYDRQGE | 33 | CUCUCAAUGGAU | 162 | RTTN |
| | DAMVAFLDGKEVDEPYTLQPPAKCHILAVSRPIEEWPIAR | | AACGAUUGCUCU | | |
| | VTMAVQEHVYALPVHEVEKSRPETTEGSRSAWFKNSGVSN | | CUACGGAGAGAC | | |
| | HGVTHAQTLNAILKNAYNVYNGVIKKVENRNAKKRDSLAA | | | | |
| | KNKSRERKGLPHFKADPPELATDEQGYLLQPPSPNSSVYL | | | | |
| | VQQHLRTPQIDLPSGYTGPVVDPRSPIPSLIPIDRLAIPP | | | | |
| | GQPGYVPLHDREKLTSNKHRRMKLPKSLRAQGALPVCFRV | | | | |
| | FDDWAVVDGRGLLRHAQYRRLAPKNVSIAELLELYTGDPV | | | | |
| | IDIKRNLMTFRFAEAVVEVTARKIVEKYHNKYLLKLTEPK | | | | |
| | GKPVREIGLVSIDLNVQRLIALAIYRVHQTGESQLALSPC | | | | |
| | LHREILPAKGLGDFDKYKSKFNQLTEEILTAAVQTLTSAQ | | | | |
| | QEEYQRYVEESSHEAKADLCLKYSITPHELAWDKMTSSTQ | | | | |
| | YISRWLRDHGWNASDFTQITKGRKKVERLWSDSRWAQELK | | | | |
| | PKLSNETRRKLEDAKHDLQRANPEWQRLAKRKQEYSRHLA | | | | |
| | NTVLSMAREYTACETVVIAIENLPMKGGFVDGNGSRESGW | | | | |
| | DNFFTHKKENRWMIKDIHKALSDLAPNRGVHVLEVNPQYT | | | | |
| | SQTCPECGHRDKANRDPIQRERFCCTHCGAQRHADLEVAT | | | | |
| | HNIAMVATTGKSLTGKSLAPQRLQEAAE | | | | |
| CasΦ.41 | VLLSDRIQYTDPSAPIPAMTVVDRRKIKKGEPGYVPPFMR | 34 | | | |
| | KNLSTNKHRRMRLSRGQKEACALPVGLRLPDGKDGWDFII | | | | |
| | FDGRALLRACRRLRLEVTSMDDVLDKFTGDPRIQLSPAGE | | | | |
| | TIVTCMLKPQHTGVIQQKLITGKMKDRLVQLTAEAPIAML | | | | |
| | TVDLGEHNLVACGAYTVGQRRGKLQSERLEAFLLPEKVLA | | | | |
| | DFEGYRRDSDEHSETLRHEALKALSKRQQREVLDMLRTGA | | | | |
| | DQARESLCYKYGLDLQALPWDKMSSNSTFIAQHLMSLGFG | | | | |
| | ESATHVRYRPKRKASERTILKYDSRFAAEEKIKLTDETRR | | | | |
| | AWNEAIWECQRASQEFRCLSVRKLQLARAAVNWTLTQAKQ | | | | |
| | RSRCPRVVVVVEDLNVRFMHGGGKRQEGWAGFFKARSEKR | | | | |
| | WFIQALHKAYTELPTNRGIHVMEVNPARTSITCTKCGYCD | | | | |
| | PENRYGEDFHCRNPKCKVRGGHVANADLDIATENLARVAL | | | | |
| | SGPMPKAPKLK | | | | |
| CasΦ.34 | MTPSFGYQMIIVTPIHHASGAWATLRLLFLNPKTSGVMLG | 35 | GUUGAACCCUCA | 163 | NTTS |
| | MTKTKSAFALMREEVFPGLLFKSADLKMAGRKFAKEGREA | | ACAGAUUGCUCG | | |
| | AIEYLRGKDEERPANFKPPAKGDIIAQSRPFDQWPIVQVS | | GUAAGCCGAGAC | | |
| | QAIQKYIFGLTKAEFDATKTLLYGEGNHPTTESRRRWFEA | | | | |
| | TGVPDFGFTSAQGLNAIFSSALARYEGVIQKVENRNEKRL | | | | |
| | KKLSEKNQRLVEEGHAVEAYVPETAFHTLESLKALSEKSL | | | | |
| | VPLDDLMDKIDRLAQPPGINPCLYGYQQVAPYIYDPENPR | | | | |
| | GVVLPDLYLGYCRKPDDPITACPNRLDIPKGQPGYIPEHQ | | | | |
| | RGQLKKHGRVRRFRYTNPQAKARAKAQTAILAVLRIDEDW | | | | |
| | VVMDLRGLLRNVYFREVAAPGELTARTLLDTFTGCPVLNL | | | | |
| | RSNVVTFCYDIESKGALHAEYVRKGWATRNKLLDLTKDGQ | | | | |
| | SVALLSVDLGQRHPVAVMISRLKRDDKGDLSEKSIQVVSR | | | | |
| | TFADQYVDKLKRYRVQYDALRKEIYDAALVSLPPEQQAEI | | | | |
| | RAYEAFAPGDAKANVLSVMFQGEVSPDELPWDKMNINTHY | | | | |
| | ISDLYLRRGGDPSRVFFVPQPSTPKKNAKKPPAPRKPVKR | | | | |
| | TDENVSHMPEFRPHLSNETREAFQKAKWTMERGNVRYAQL | | | | |
| | SRFLNQIVREANNWLVSEAKKLTQCQTVVWAIEDLHVPFF | | | | |
| | HGKGKYHETWDGFFRQKKEDRWFVNVFHKAISERAPNKGE | | | | |
| | YVMEVAPYRTSQRCPVCGFVDADNRHGDHFKCLRCGVELH | | | | |
| | ADLEVATWNIALVAVQGHGIAGPPREQSCGGETAGTARKG | | | | |
| | KNIKKNKGLADAVTVEAQDSEGGSKKDAGTARNPVYIPSE | | | | |
| | SQVNCPAP | | | | |
| CasΦ.35 | MKPKTPKPPKTPVAALIDKHFPGKRFRASYLKSVGKKLKN | 36 | | | NTTS |
| | QGEDVAVRFLTGKDEERPPNFQPPAKSNIVAQSRPIEEWP | | | | |
| | IHKVSVAVQEYVYGLTVAEKEACSDAGESSSSHAAWFAKT | | | | |
| | GVENFGYTSVQGLNKIFPPTFNRFDGVIKKVENRNEKKRQ | | | | |
| | KATRINEAKRNKGQSEDPPEAEVKATDDAGYLLQPPGINH | | | | |
| | SVYGYQSITLCPYTAEKFPTIKLPEEYAGYHSNPDAPIPA | | | | |
| | GVPDRLAIPEGQPGHVPEEHRAGLSTKKHRRVRQWYAMAN | | | | |
| | WKPKPKRTSKPDYDRLAKARAQGALLIVIRIDEDWVVVDA | | | | |
| | RGLLRNVRWRSLGKREITPNELLDLFTGDPVLDLKRGVVT | | | | |
| | FTYAEGVVNVCSRSTTKGKQTKVLLDAMTAPRDGKKRQIG | | | | |
| | MVAVDLGQTNPIAAEYSRVGKNAAGTLEATPLSRSTLPDE | | | | |
| | LLREIALYRKAHDRLEAQLREEAVLKLTAEQQAENARYVE | | | | |
| | TSEEGAKLALANLGVDTSTLPWDAMTGWSTCISDHLINHG | | | | |
| | GDTSAVFFQTIRKGTKKLETIKRKDSSWADIVRPRLTKET | | | | |
| | REALNDFLWELKRSHEGYEKLSKRLEELARRAVNHVVQEV | | | | |
| | KWLTQCQDIVIVIEDLNVRNFHGGGKRGGGWSNFFTVKKE | | | | |
| | NRWFMQALHKAFSDLAAHRGIPVLEVYPARTSITCLGCGH | | | | |
| | CDPENRDGEAFVCQQCGATFHADLEVATRNIARVALTGEA | | | | |
| | MPKAPAREQPGGAKKRGTSRRRKLTEVAVKSAEPTIHQAK | | | | |
| | NQQLNGTSRDPVYKGSELPAL | | | | |
| CasΦ.43 | MSEITDLLKANFKGKTFKSADMRMAGRILKKSGAQAVIKY | 37 | | | NTTN |
| | LSDKGAVDPPDFRPPAKCNIIAQSRPFDEWPICKASMAIQ | | | | |
| | QHIYGLTKNEFDESSPGISSASHEQWFAKTGVDTHGFTHV | | | | |
| | QGLNLIFQHAKKRYEGVIKKVENYNEKERKKFEGINERRS | | | | |
| | KEGMPLLEPRLRTAFGDDGKFAEKPGVNPSIYLYQQTSPR | | | | |
| | PYDKTKHPYVHAPFELKEITTIPTQDDRLKIPFGAPGHVP | | | | |
| | EKHRSQLSMAKHKRRRAWYALSQNKPRPPKDGSKGRRSVR | | | | |
| | DLADLKAASLADAIPLVSRVGFDWVVIDGRGLLRNLRWRK | | | | |
| | LAHEGMTVEEMLGFFSGDPVIDPRRNVATFIYKAEHATVK | | | | |
| | SRKPIGGAKRAREELLKATASSDGVIRQVGLISVDLGQTN | | | | |
| | PVAYEISRMHQANGELVAEHLEYGLLNDEQVNSIQRYRAA | | | | |
| | WDSMNESFRQKAIESLSMEAQDEIMQASTGAAKRTREAVL | | | | |
| | TMFGPNATLPWSRMSSNTTCISDALIEVGKEEETNFVTSN | | | | |
| | GPRKRTDAQWAAYLRPRVNPETRALLNQAVWDLMKRSDEY | | | | |
| | ERLSKRKLEMARQCVNFVVARAEKLTQCNNIGIVLENLVV | | | | |
| | RNFHGSGRRESGWEGFFEPKRENRWFMQVLHKAFSDLAQH | | | | |
| | RGVMVFEVHPAYSSQTCPACRYVDPKNRSSEDRERFKCLK | | | | |
| | CGRSFNADREVATFNIREIARTGVGLPKPDCERSRGVQTT | | | | |
| | GTARNPGRSLKSNKNPSEPKRVLQSKTRKKITSTETQNEP | | | | |
| | LATDLKT | | | | |
| CasΦ.44 | MTPKTESPLSALCKKHFPGKRFRTNYLKDAGKILKKHGED | 38 | | | NTTS |
| | AVVAFLSDKQEDEPANFCPPAKVHILAQSRPFEDWPINLA | | | | |
| | SKAIQTYVYGLTADERKTCEPGTSKESHDRWFKETGVDHH | | | | |
| | GFTSVQGLNLIFKHILNRYDGVIKKVETRNEKRRSSVVRI | | | | |
| | NEKKAAEGLPLIAAEAEETAFGEDGRLLQPPGVNHSIYCF | | | | |
| | QQVSPQPYSSKKHPQVVLPHAVQGVDPDAPIPVGRPNRLD | | | | |
| | IPKGQPGYVPEWQRPHLSMKCKRVRMWYARANWRRKPGRR | | | | |
| | SVLNEARLKEASAKGALPIVLVIGDDWLVMDARGLLRSVF | | | | |
| | WRRVAKPGLSLSELLNVTPTGLFSGDPVIDPKRGLVTFTS | | | | |
| | KLGVVAVHSRKPTRGKKSKDLLLKMTKPTDDGMPRHVGMV | | | | |
| | AIDLGQTNPVAAEYSRVVQSDAGTLKQEPVSRGVLPDDLL | | | | |
| | KDVARYRRAYDLTEESIRQEAIALLSEGHRAEVTKLDQTT | | | | |
| | ANETKRLLVDRGVSESLPWEKMSSNTTYISDCLVALGKTD | | | | |
| | DVFFVPKAKKGKKETGIAVKRKDHGWSKLLRPRTSPEARK | | | | |
| | ALNENQWAVKRASPEYERLSRRKLELGRRCVNHIIQETKR | | | | |
| | WTQCEDIVVVLEDLNVGFFHGSGKRPDGWDNFFVSKRENR | | | | |
| | WFIQVLHKAFGDLATHRGTHVIEVHPARTSITCIKCGHCD | | | | |
| | AGNRDGESFVCLASACGDRRHADLEVATRNVARVAITGER | | | | |
| | MPPSEQARDVQKAGGARKRKPSARNVKSSYPAVEPAPASP | | | | |
| CasΦ.36 | MSDNKMKKLSKEEKPLTPLQILIRKYIDKSQYPSGFKTTI | 39 | | | |
| | IKQAGVRIKSVKSEQDEINLANWIISKYDPTYIKRDENPS | | | | |
| | AKCQIIATSRSVADFDIVKMSNKVQEIFFASSHLDKNVED | | | | |
| | IGKSKSDHDSWFERNNVDRGIYTYSNVQGMNLIFSNTKNT | | | | |
| | YLGVAVKAQNKFSSKMKRIQDINNFRITNHQSPLPIPDEI | | | | |
| | KIYDDAGFLLNPPGVNPNIFGYQSCLLKPLENKEIISKTS | | | | |
| | FPEYSRLPADMIEVNYKISNRLKFSNDQKGFIQFKDKLNL | | | | |
| | FKINSQELFSKRRRLSGQPILLVASFGDDWVVLDGRGLLR | | | | |
| | QVYYRGIAKPGSITISELLGFFTGDPIVDPIRGVVSLGFK | | | | |
| | PGVLSQETLKTTSARIFAEKLPNLVLNNNVGLMSIDLGQT | | | | |
| | NPVSYRLSEITSNMSVEHICSDFLSQDQISSIEKAKTSLD | | | | |
| | NLEEEIAIKAVDHLSDEDKINFANFSKLNLPEDTRQSLFE | | | | |
| | KYPELIGSKLDFGSMGSGTSYIADELIKFENKDAFYPSGK | | | | |
| | KKFDLSFSRDLRKKLSDETRKSYNDALFLEKRINDKYLKN | | | | |
| | AKRRKQIVRTVANSLVSKIEELGLTPVINIENLAMSGGFF | | | | |
| | DGRGKREKGWDNFFKVKKENRWVMKDFHKAFSELSPHHGV | | | | |
| | IVIESPPYCTSVTCTKCNFCDKKNRNGHKFTCQRCGLDAN | | | | |
| | ADLDIATENLEKVAISGKRMPGSERSSDERKVAVARKAKS | | | | |
| | PKGKAIKGVKCTITDEPALLSANSQDCSQSTS | | | | |
| CasΦ.37 | MALSLAEVRERHFKGLRFRSSYLKRAGKILKKEGEAACVA | 40 | | | NTTN |
| | YLTGKDEESPPNFKPPAKCDVVAQSRPFEEWPIVQASVAV | | | | |
| | QSYVYGLTKEAFEAFNPGTTKQSHEACLAATGIDTCGYSN | | | | |
| | VQGLNLIFRQAKNRYEGVITKVENRNKKAKKKLTRKNEWR | | | | |
| | QKNGHSELPEAPEELTENDEGRLLQPPGINPSLYTYQQIS | | | | |
| | PTPWSPKDSSILPPQYAGYERDPNAPIPFGVAKDRLTIAS | | | | |
| | GCPGYIPEWMRTAGEKTNPRTQKKFMHPGLSTRKNKRMRL | | | | |
| | PRSVRSAPLGALLVTIHLGEDWLVLDVRGLLRNARWRGVA | | | | |
| | PKDISTQGLLNLFTGDPVIDTRRGVVTFTYKPETVGIHSR | | | | |
| | TWLYKGKQTKEVLEKLTQDQTVALVAIDLGQTNPVSAAAS | | | | |
| | RVSRSGENLSIETVDRFFLPDELIKELRLYRMAHDRLEER | | | | |
| | IREESTLALTEAQQAEVRALEHVVRDDAKNKVCAAFNLDA | | | | |
| | ASLPWDQMTSNTTYLSEAILAQGVSRDQVFFTPNPKKGSK | | | | |
| | EPVEVMRKDRAWVYAFKAKLSEETRKAKNEALWALKRASP | | | | |
| | DYARLSKRREELCRRSVNMVINRAKKRTQCQVVIPVLEDL | | | | |
| | NIGFFHGSGKRLPGWDNFFVAKKENRWLMNGLHKSFSDLA | | | | |
| | VHRGFYVFEVMPHRTSITCPACGHCDSENRDGEAFVCLSC | | | | |
| | KRTYHADLDVATHNLTQVAGTGLPMPEREHPGGTKKPGGS | | | | |
| | RKPESPQTHAPILHRTDYSESADRLGS | | | | |
| CasΦ.45 | QAVIKYLSDKGAVDPPDFRPPAKCNIIAQSRPFDEWPICK | 41 | ACUGAAACCACC | 164 | NTTN |
| | ASMAIQQHIYGLTKNEFDESSPGTSSASHEQWFAKTGVDT | | AACGAUUGCGCU | | |
| | HGFTHVQGLNLIFQHAKKRYEGVIKKVENYNEKERKKFEG | | CCUCGGAGCGAC | | |
| | INERRSKEGMPLLEPRLRTAFGDDGKFAEKPGVNPSIYLY | | | | |
| | QQTSPRPYDKTKHPYVHAPFELKEITTIPTQDDRLKIPFG | | | | |
| | APGHVPEKHRSQLSMAKHKRRRAWYALSQNKPRPPKDGSK | | | | |
| | GRRSVRDLADLKAASLADAIPLVSRVGFDWVVIDGRGLLR | | | | |
| | NLRWRKLAHEGMTVEEMLGFFSGDPVIDPRRNVATFIYKA | | | | |
| | EHATVKSRKPIGGAKRAREELLKATASSDGVIRQVGLISV | | | | |
| | DLGQTNPVAYEISRMHQANGELVAEHLEYGLINDEQVNSI | | | | |
| | QRYRAAWDSMNESFRQKAIESLSMEAQDEIMQASTGAAKR | | | | |
| | TREAVLTMFGPNATLPWSRMSSNTTCISDALIEVGKEEET | | | | |
| | NFVTSNGPRKRTDAQWAAYLRPRVNPETRALLNQAVWDLM | | | | |
| | KRSDEYERLSKRKLEMARQCVNFVVARAEKLTQCNNIGIV | | | | |
| | LENLVVRNFHGSGRRESGWEGFFEPKRENRWFMQVLHKAF | | | | |
| | SDLAQHRGVMVFEVHPAYSSQTCPACRYVDPKNRSSEDRE | | | | |
| | RFKCLKCGRSFNADREVATFNIREIARTGVGLPKPDCERS | | | | |
| | RDVQTPGTARKSGRSLKSQDNLSEPKRVLQSKTRKKITST | | | | |
| | ETQNEPLATDLKT | | | | |
| CasΦ.38 | MIKEQSELSKLIEKYYPGKKFYSNDLKQAGKHLKKSEHLT | 42 | | | NTWN |
| | AKESEELTVEFLKSCKEKLYDFRPPAKALIISTSRPFEEW | | | | |
| | PIYKASESIQKYIYSLTKEELEKYNISTDKTSQENFFKES | | | | |
| | LIDNYGFANVSGLNLIFQHTKAIYDGVLKKVNNRNNKILK | | | | |
| | KYKRKIEEGIEIDSPELEKAIDESGHFINPPGINKNIYCY | | | | |
| | QQVSPTIFNSFKETKIICPFNYKRNPNDIIQKGVIDRLAI | | | | |
| | PFGEPGYIPDHQRDKVNKHKKRIRKYYKNNENKNKDAILA | | | | |
| | KINIGEDWVLFDLRGLLRNAYWRKLIPKQGITPQQLLDMF | | | | |
| | SGDPVIDPIKNNITFIYKESIIPIHSESIIKTKKSKELLE | | | | |
| | KLTKDEQIALVSIDLGQTNPVAARFSRLSSDLKPEHVSSS | | | | |
| | FLPDELKNEICRYREKSDLLEIEIKNKAIKMLSQEQQDEI | | | | |
| | KLVNDISSEELKNSVCKKYNIDNSKIPWDKMNGFTTFIAD | | | | |
| | EFINNGGDKSLVYFTAKDKKSKKEKLVKLSDKKIANSFKP | | | | |
| | KISKETREILNKITWDEKISSNEYKKLSKRKLEFARRATN | | | | |
| | YLINQAKKATRLNNVVLVVEDLNSKFFHGSGKREDGWDNF | | | | |
| | FIPKKENRWFIQALHKSLTDVSIHRGINVIEVRPERTSIT | | | | |
| | CPKCGCCDKENRKGEDFKCIKCDSVYHADLEVATFNIEKV | | | | |
| | AITGESMPKPDCERLGGEESIG | | | | |
| CasΦ.39 | VAFLDGKEVDEPYTLQPPAKCHILAVSRPIEEWPIARVTM | 43 | | | RTTN |
| | AVQEHVYALPVHEVEKSRPETTEGSRSAWFKNSGVSNHGV | | | | |
| | THAQTLNAILKNAYNVYNGVIKKVENRNAKKRDSLAAKNK | | | | |
| | SRERKGLPHFKADPPELATDEQGYLLQPPSPNSSVYLVQQ | | | | |
| | HLRTPQIDLPSGYTGPVVDPRSPIPSLIPIDRLAIPPGQP | | | | |
| | GYVPLHDREKLTSNKHRRMKLPKSLRAQGALPVCFRVEDD | | | | |
| | WAVVDGRGLLRHAQYRRLAPKNVSIAELLELYTGDPVIDI | | | | |
| | KRNLMTFRFAEAVVEVTARKIVEKYHNKYLLKLTEPKGKP | | | | |
| | VREIGLVSIDLNVQRLIALAIYRVHQTGESQLALSPCLHR | | | | |
| | EILPAKGLGDFDKYKSKFNQLTEEILTAAVQTLTSAQQEE | | | | |
| | YQRYVEESSHEAKADLCLKYSITPHELAWDKMTSSTQYIS | | | | |
| | RWLRDHGWNASDFTQITKGRKKVERLWSDSRWAQELKPKL | | | | |
| | SNETRRKLEDAKHDLQRANPEWQRLAKRKQEYSRHLANTV | | | | |
| | LSMAREYTACETVVIAIENLPMKGGFVDGNGSRESGWDNF | | | | |
| | FTHKKENRWMIKDIHKALSDLAPNRGVHVLEVNPQYTSQT | | | | |
| | CPECGHRDKANRDPIQRERFCCTHCGAQRHADLEVATHNI | | | | |
| | AMVATTGKSLTGKSLAPQRLQ | | | | |
| CasΦ.42 | LEIPEGEPGHVPWFQRMDIPEGQIGHVNKIQRFNFVHGKN | 44 | | | |
| | SGKVKFSDKTGRVKRYHHSKYKDATKPYKFLEESKKVSAL | | | | |
| | DSILAIITIGDDWVVFDIRGLYRNVFYRELAQKGLTAVQL | | | | |
| | LDLFTGDPVIDPKKGIITFSYKEGVVPVFSQKIVSRFKSR | | | | |
| | DTLEKLTSQGPVALLSVDLGQNEPVAARVCSLKNINDKIA | | | | |
| | LDNSCRIPFLDDYKKQIKDYRDSLDELEIKIRLEAINSLD | | | | |
| | VNQQVEIRDLDVFSADRAKASTVDMFDIDPNLISWDSMSD | | | | |
| | ARFSTQISDLYLKNGGDESRVYFEINNKRIKRSDYNISQL | | | | |
| | VRPKLSDSTRKNLNDSIWKLKRTSEEYLKLSKRKLELSRA | | | | |
| | VVNYTIRQSKLLSGINDIVIILEDLDVKKKENGRGIRDIG | | | | |
| | WDNFFSSRKENRWFIPAFHKSFSELSSNRGLCVIEVNPAW | | | | |
| | TSATCPDCGFCSKENRDGINFTCRKCGVSYHADIDVATLN | | | | |
| | IARVAVLGKPMSGPADRERLGGTKKPRVARSRKDMKRKDI | | | | |
| | SNGTVEVMVTA | | | | |
| CasΦ.46 | IPSFGYLDRLKIAKGQPGYIPEWQRETINPSKKVRRYWAT | 45 | | | |
| | NHEKIRNAIPLVVFIGDDWVIIDGRGLLRDARRRKLADKN | | | | |
| | TTIEQLLEMVSNDPVIDSTRGIATLSYVEGVVPVRSFIPI | | | | |
| | GEKKGREYLEKSTQKESVTLLSVDIGQINPVSCGVYKVSN | | | | |
| | GCSKIDFLDKFFLDKKHLDAIQKYRTLQDSLEASIVNEAL | | | | |
| | DEIDPSFKKEYQNINSQTSNDVKKSLCTEYNIDPEAISWQ | | | | |
| | DITAHSTLISDYLIDNNITNDVYRTVNKAKYKINDFGWYK | | | | |
| | KFSAKLSKEAREALNEKIWELKIASSKYKKLSVRKKEIAR | | | | |
| | TIANDCVKRAETYGDNVVVAMESLTKNNKVMSGRGKRDPG | | | | |
| | WHNLGQAKVENRWFIQAISSAFEDKATHHGTPVLKVNPAY | | | | |
| | TSQTCPSCGHCSKDNRSSKDRTIFVCKSCGEKFNADLDVA | | | | |
| | TYNIAHVAFSGKKLSPPSEKSSATKKPRSARKSKKSRKS | | | | |
| CasΦ.47 | SPIEKLLNGLLVKITFGNDWIICDARGLLDNVQKGIIHKS | 46 | | | |
| | YFTNKSSLVDLIDLFTCNPIVNYKNNVVTFCYKEGVVDVK | | | | |
| | SFTPIKSGPKTQENLIKKLKYSRFQNEKDACVLGVGVDVG | | | | |
| | VTNPFAINGFKMPVDESSEWVMLNEPLFTIETSQAFREEI | | | | |
| | MAYQQRTDEMNDQFNQQSIDLLPPEYKVEFDNLPEDINEV | | | | |
| | AKYNLLHTLNIPNNFLWDKMSNTTQFISDYLIQIGRGTET | | | | |
| | EKTITTKKGKEKILTIRDVNWFNTFKPKISEETGKARTEI | | | | |
| | KRDLQKNSDQFQKLAKSREQSCRTWVNNVTEEAKIKSGCP | | | | |
| | LIIFVIEALVKDNRVFSGKGHRAIGWHNFGKQKNERRWWV | | | | |
| | QAIHKAFQEQGVNHGYPVILCPPQYTSQTCPKCNHVDRDN | | | | |
| | RSGEKFKCLKYGWIGNADLDVGAYNIARVAITGKALSKPL | | | | |
| | EQKKIKKAKNKT | | | | |
| CasΦ.48 | LLDNVQKGIIHKSYFTNKSSLVDLIDLFTCNPIVNYKNNV | 47 | | | |
| | VTFCYKEGVVDVKSFTPIKSGPKTQENLIKKLKYSRFQNE | | | | |
| | KDACVLGVGVDVGVTNPFAINGFKMPVDESSEWVMLNEPL | | | | |
| | FTIETSQAFREEIMAYQQRTDEMNDQFNQQSIDLLPPEYK | | | | |
| | VEFDNLPEDINEVAKYNLLHTLNIPNNFLWDKMSNTTQFI | | | | |
| | SDYLIQIGRGTETEKTITTKKGKEKILTIRDVNWFNTFKP | | | | |
| | KISEETGKARTEIKRDLQKNSDQFQKLAKSREQSCRTWVN | | | | |
| | NVTEEAKIKSGCPLIIFVIEALVKDNRVFSGKGHRAIGWH | | | | |
| | NFGKQKNERRWWVQAIHKAFQEQGVNHGYPVILCPPQYTS | | | | |
| | QTCPKCNHVDRDNRSGEKFKCLKYGWIGNADLDVGAYNIA | | | | |
| | RVAITGKALSKPLEQKKIKKAKNKT | | | | |
| CasΦ.49 | MIKPTVSQFLTPGFKLIRNHSRTAGLKLKNEGEEACKKFV | 48 | | | |
| | RENEIPKDECPNFQGGPAIANIIAKSREFTEWEIYQSSLA | | | | |
| | IQEVIFTLPKDKLPEPILKEEWRAQWLSEHGLDTVPYKEA | | | | |
| | AGLNLIIKNAVNTYKGVQVKVDNKNKNNLAKINRKNEIAK | | | | |
| | LNGEQEISFEEIKAFDDKGYLLQKPSPNKSIYCYQSVSPK | | | | |
| | PFITSKYHNVNLPEEYIGYYRKSNEPIVSPYQFDRLRIPI | | | | |
| | GEPGYVPKWQYTFLSKKENKRRKLSKRIKNVSPILGIICI | | | | |
| | KKDWCVFDMRGLLRTNHWKKYHKPTDSINDLFDYFTGDPV | | | | |
| | IDTKANVVRFRYKMENGIVNYKPVREKKGKELLENICDQN | | | | |
| | GSCKLATVDVGQNNPVAIGLFELKKVNGELTKTLISRHPT | | | | |
| | PIDFCNKITAYRERYDKLESSIKLDAIKQLTSEQKIEVDN | | | | |
| | YNNNFTPQNTKQIVCSKLNINPNDLPWDKMISGTHFISEK | | | | |
| | AQVSNKSEIYFTSTDKGKTKDVMKSDYKWFQDYKPKLSKE | | | | |
| | VRDALSDIEWRLRRESLEFNKLSKSREQDARQLANWISSM | | | | |
| | CDVIGIENLVKKNNFFGGSGKREPGWDNFYKPKKENRWWI | | | | |
| | NAIHKALTELSQNKGKRVILLPAMRTSITCPKCKYCDSKN | | | | |
| | RNGEKFNCLKCGIELNADIDVATENLATVAITAQSMPKPT | | | | |
| | CERSGDAKKPVRARKAKAPEFHDKLAPSYTVVLREAVKRP | | | | |
| | AATKKAGQAKKKKEF | | | | |
| CasΦ.12 | SNAPKKKRKVGIHGVPAAMIKPTVSQFLTPGFKLIRNHSR | 49 | | | |
| with NLS | TAGLKLKNEGEEACKKFVRENEIPKDECPNFQGGPAIANI | | | | |
| Signals | IAKSREFTEWEIYQSSLAIQEVIFTLPKDKLPEPILKEEW | | | | |
| | RAQWLSEHGLDTVPYKEAAGLNLIIKNAVNTYKGVQVKVD | | | | |
| | NKNKNNLAKINRKNEIAKLNGEQEISFEEIKAFDDKGYLL | | | | |
| | QKPSPNKSIYCYQSVSPKPFITSKYHNVNLPEEYIGYYRK | | | | |
| | SNEPIVSPYQFDRLRIPIGEPGYVPKWQYTFLSKKENKRR | | | | |
| | KLSKRIKNVSPILGIICIKKDWCVFDMRGLLRTNHWKKYH | | | | |
| | KPTDSINDLFDYFTGDPVIDTKANVVRFRYKMENGIVNYK | | | | |
| | PVREKKGKELLENICDQNGSCKLATVDVGQNNPVAIGLFE | | | | |
| | LKKVNGELTKTLISRHPTPIDFCNKITAYRERYDKLESSI | | | | |
| | KLDAIKQLTSEQKIEVDNYNNNFTPQNTKQIVCSKLNINP | | | | |
| | NDLPWDKMISGTHFISEKAQVSNKSEIYFTSTDKGKTKDV | | | | |
| | MKSDYKWFQDYKPKLSKEVRDALSDIEWRLRRESLEFNKL | | | | |
| | SKSREQDARQLANWISSMCDVIGIENLVKKNNFFGGSGKR | | | | |
| | EPGWDNFYKPKKENRWWINAIHKALTELSQNKGKRVILLP | | | | |
| | AMRTSITCPKCKYCDSKNRNGEKFNCLKCGIELNADIDVA | | | | |
| | TENLATVAITAQSMPKPTCERSGDAKKPVRARKAKAPEFH | | | | |
| | DKLAPSYTVVLREAVKRPAATKKAGQAKKKKEF | | | | |
| CasM.29 | MAKKGTNRKKMIVKVMKYELKYESGCADFNEMQNELWKLQ | 50 | CGUUGCAGCUCG | 165; | NCTT |
| 8706 | RQTREVMNRTIQLCYHWSYVQADYCKQHGCARRDVKPCDV | | CACGUUGGCACU | 166; | |
| | YETNATSLDGYIYQLFKDEYPNFLMANLIATLRKAHQKYD | | GGUUGAAGG; | 167; | |
| | ALLFDIQEGNSSIPSFKKDQPLIFSKEAIRLPECLSDKRQ | | CGUUGCAGCUCG | 168 | |
| | ITLFCFSKPYKSAHPTLDKITFAVRARSASEKSIFDHIIS | | CACGUUGGCACU | | |
| | GKYALGESQLVYEKKKWFFLLSYKFTPESVDVNPEKVLGV | | GGGUUGAAGG; | | |
| | DLGVVNALCAGSVENPHDSLFIKGTEAIEQIRRLEARKRD | | UUGGCACUGGUU | | |
| | LQKQARYPGDGRIGHGTKTRVSPVYQTRDAIARMQDTLNH | | GAAGG; | | |
| | RWSRALIDFACKKGYGTIQMEDLSGIKALESEKPYLKHWT | | CACUGGUUGAAG | | |
| | YFDLQSKIIYKAEEKGIRVVKVNPKCTSRRCSACGYISKE | | G | | |
| | NRKNQVEFLCVNCGYHHNADYNAAQNLSIPQIDRLIEKQL | | | | |
| | KEQESEENEAGANPK | | | | |
| CasM.28 | MAKGTLSKVMKYELRYLDGCGDFQNMQKELWTLQRQSREI | 51 | GUUGCAACUCAC | 169 | |
| 0604 | LNRTIQIAYHWDYTDREQFKKTGQHLDIKAETGYKRLDGY | | GCGCGUAUGUGG | | |
| | IYDSLKEDVQNFASVNVNATIQKAWAKYKSSKIDVLRGDM | | CUUGAAGG | | |
| | SLPSYKSDQPLVLHAQSMKIFSSDDDDVLQVTLFSNAYKK | | | | |
| | ACNYSNIRFIIGLHDATQRTIIKKVLSGDWGIGQSQIVYK | | | | |
| | RPKWFLYLTYNFSPEQHEVNPDKILGVDLGESIAIYASSI | | | | |
| | GEYGSLRIEGGEISAFAKQLEARKRSLQKQAAYCGKGRIG | | | | |
| | HGTKSRVSDVYKMEDKIANFRNTVNHRYSKMLIDYALKHM | | | | |
| | YGTIQMEDLSGIKKETGFPKFLQHWTYYDLQQKIEAKAKE | | | | |
| | HGINFIKVDPAFTSQRCSKCGNIDSENRPSQAVFCCKKCG | | | | |
| | YKTNADFNAS | | | | |
| CasM.28 | MNVTKVMRYQLIYQGGGGDFESLQNQLWEFQRQTRAILNK | 52 | GUUGCAAUUCAU | 170 | |
| 1060 | TIQTMYLATANQEKFSEKALYHDLCAEYPDMISSTVNATL | | AUCUCCGGGUGG | | |
| | REATKKYRSSVREILAGRMSLPSYKRDHPILLHNQSVALK | | AUUGAAGG | | |
| | QGNQGSYFATISVFSRKYQQGTPGVKQPSFQLIAKDNTQR | | | | |
| | TILQRLLSGEYKLGQCQLIYIRPKWFLNVAYSFTPSEKAL | | | | |
| | DQEKVLGVDLGCVYAIYASSYGNHGIFKISGDEITSFERK | | | | |
| | QAAIQNRAFKNDLTRIREIEERRKQKLEQARYCGEGRIGH | | | | |
| | GVKTRVAPAYQDEGKISRFRETINHRYSKALVDYAEKNGY | | | | |
| | GTIQMEDLSGIKSSTGFPKRLQHWTYFDLQQKIKYKAEEQ | | | | |
| | GIKVVKIKPAYTSQRCSRCGHIDPANRKSQSEFKCIACGF | | | | |
| | SSNADYNASQNISMRNIEKIIQGKAN | | | | |
| CasM.28 | MAKGTITKVMKYELRYLGGFSDFHEMQKEVWQLQRQYREI | 53 | GUUGCAGCGUGC | 171; | NTTC; TTTC |
| 4933 | LNKTIQIALHWDYVSAQQFGESGTYLDIREETGYKTLDGY | | GCGAGCGUGUGG | 172; | |
| | IYNCLKGAYSEMASANLNAAVQKAWKKYKNSKTQVLQGVM | | CUUGAAGG; | 173 | |
| | SLPSYKSDQPILIDKGNVKLSAEENNGRAVLTLFSRNYRD | | AGCGUGUGGCUU | | |
| | TRGLKGNVEFSVLLHDGTQKSIFRNLIDKTYALGQCQLVY | | GAAGG; | | |
| | ERKKWFLLLTYSFTPAGHALDPEKILGVDLGECYALYASS | | UGUGGCUUGAAG | | |
| | CYAPGILKIEGGEIAEYALRLEKRKRSLQQQARYCGEGRI | | G | | |
| | GHGTKTRVGVVYKAEDRIASFRETINHRYSKELVDYAVSN | | | | |
| | GYGTIQMEDLSAIQKDLGFPKRLRHWTYYDLQMKITNKAK | | | | |
| | EHGIAVVKIDPRYTSQRCSKCGHIDPANRPRQEEFCCTAC | | | | |
| | GYACNADYNASQNISIKGIEKIIQKMLSAKAD | | | | |
| CasM.28 | MSKGMLTKVMKYTLRYVGGCGDFHEMQSILWELQKQTRAV | 54 | GUUGCAACUCGC | 174; | NTTY |
| 7908 | LNKTIQIAFEWDYRSREAFQETGEYLDVHAETGYKRLDGY | | ACGUGAAUGCGA | 175 | |
| | IYNCLKNEYADFAGKNLNAAIQTAWKKYNQSKRDIQTGKM | | CUUGAAGG; | | |
| | SLPSYRSNQPLIIHNDNVMISQDMQAAPSVRFTLLSLEYK | | UGAAUGCGACUU | | |
| | KAHDLNTNPTFEVLINDGTQRAIFEKVRSGEYKLGQCMIQ | | GAAGG | | |
| | YDKKKWFLLLTYSFQPEKLTLDKNKILGVDLGETIVICAS | | | | |
| | SVSERGRFVIDGGEITRFATQIEARKRSQQHQAAYCGEGR | | | | |
| | IGHGTKTRVDAVYKTEDRIANFRDTINHRYSRALVNYAVK | | | | |
| | HGFGTIQMEDLSGIKSSDDFPKFLRHWTYYDLQSKIESKA | | | | |
| | KERGIAVVKVNPRFTSRRCSKCGYIDEGNRKDQAHFCCLS | | | | |
| | CGFRANADFNASQNLSIKGIDKIIEKEYNANSKQT | | | | |
| CasM.28 | MGKPITKTMKYQIHYIDGCGDFHNMQKELWDLQRIVRQIL | 55 | GAUGCAACUCGU | 176 | |
| 8518 | NKTINESYLWFVRSEQYYRDTGENLSVEEQTGYKTLDGHI | | GUGUAUGUGCGA | | |
| | YNLLKQEYTQKLVSNSLNASIQAAYKKMKDSRRDVMIGTM | | GUUGAAGG | | |
| | SLPSYRSDQPIIIYNKNIKFSSHPEHGFVVDCSLFSDAYK | | | | |
| | KSQGYEKSVKFQVSVDDNTQRSIFENILTGNYKHGQCSIV | | | | |
| | YEKKKWFLLLTYSFVPEETKLDPDKILGVDVGVVYALYAS | | | | |
| | SKGNHGTFKIKGDEAITFIQRVEARKHSRQLQGTYCGDGR | | | | |
| | IGHGTKTRVQPVYNERALISNFQDTINHRYSKALIDYAKK | | | | |
| | NGYGTIQMEDLSGIKEVQQYPKYLQHWTYYDLQLKIQYKA | | | | |
| | KEAGIGFVKVTPKYTSQRCSHCGNIDEANRPKQDVFRCTV | | | | |
| | CGYERNADYNASQNLSIKGIDRIIDDQLKQMNKANPKKTE | | | | |
| | NA | | | | |
| CasM.29 | MSGGAITKVMKYDLTYKDGYGNFKDMQEAVWKLIRDIRTI | 56 | GACGCAACUCGC | 177 | |
| 3891 | LNETIKIAYHWDYLNEKSKRETGEHLDLLEETGYKRLDGY | | GCGCGGGCAUGU | | |
| | IYDDLKDRFPDFASSNLNAAIQTAWKKYKQSQKDVYIGKM | | AUUGAGGG | | |
| | TLPSYKSDQPLPINKQSIKIYDEEREHIVELNLFSTKHKK | | | | |
| | EHGLASNVRFRINLHDNTQHAIYERVLSGEYTLGQCQLLY | | | | |
| | DRPKWFFILTYSFKPAQNKLDPDKILGVDMGETCALYAST | | | | |
| | FGEQGSFVINGGEVSEYAKREEARKRSLQKQAAVCGEGRI | | | | |
| | GHGTKTRVSSVYKEQERISNERDTINHRYSKALIEYAVKN | | | | |
| | GCGTIQMEDLSGIRQSTDFPKFLRHWTYYDLQQKIKTKAK | | | | |
| | ETGIAVSMIDPRYTSQRCSRCGHIDKANRKDQAHFHCLKC | | | | |
| | GYSCNADFNASQNISIRGIDKIIQKELGAKAKQTD | | | | |
| CasM.29 | MKEIAKVMKYQLIYLDGGGDFYELQQTLWDLQRQTREILN | 57 | GAUGCAUCUGAC | 178; | NTTC; NYTN |
| 4270 | KTIQSMYLATATNTAFEENALYHRFGAEYPMMAALNVNAT | | ACAGCUGGGUGA | 179 | |
| | LRTAKKRYTSTIKETLRGTMSLPSYKRDQPILLHNQTIHL | | GUUGAAGG; | | |
| | ALEDGQYSALFSVYSEKFQKAHEGVARPRFALMARDGTQR | | GCUGGGUGAGUU | | |
| | AILDRLLDGSYRLGQSQMTYEQKKWFLSLTYKFVPEVREL | | GAAGG | | |
| | DKSKILGVDLGCVYAIYASSMQQKGIFKISGDEITEFEKR | | | | |
| | QAAMQNREPVSTLERVEQLEQRRWQKQQQARYCGEGRVGH | | | | |
| | GTGTRVAPAYRDADKIARFRDTINHRYSKALVEYAEKNGF | | | | |
| | GTIQMEDLSGIKEDTGFPKRLRHWTYFDLQTKIQYKAAER | | | | |
| | GITVVKIDPQYTSQRCSRCGYIDKANRASQEKFLCQSCGF | | | | |
| | EANADYNASQNISVEKIDKLIAKDKKKLART | | | | |
| CasM.29 | MGQVTKVMRYQLIYQDGGGDFYTVQQELWELQRQTREILN | 58 | GUUGCAACACAU | 180 | GNNN |
| 4491 | KTIQTMYLADANKEKFDNAAERTLNRRFCVDHPDMYTKTV | | GUAUGUGGGUGA | | |
| | TATLRKAKAKYNASQKEILAGRMSLPSYKRDQPILLNPQG | | GUUGAAGG | | |
| | FKIEEESDSFFAAIAVFSDKYKNKHPDVDVKRLRFRLVVK | | | | |
| | DGTQRAIIRRVISGEYKLGRSQLLYSKKKWFLNVTYSFEP | | | | |
| | AEKKVDPDKILGVDLGCVYAIYASSFGSPGVFKISGDEVS | | | | |
| | SFERKQAAIQNRSPKSTLERVEKIEERHKQKQQQARYCGE | | | | |
| | GRIGHGTKTRIAPVYQDEDKIARFRDTVNHRYSKALIDYA | | | | |
| | EKNGYGTIQMEDLSGIKSATGFPKRLKHWTYYDLQTKIEY | | | | |
| | KAEERGIKVVKIDPRYTSQRCSRCGYIDSGNRKSQAEFCC | | | | |
| | MACGFSCNADYNASQNISIGGIAKIIADKRKEADAK | | | | |
| CasM.29 | YLDIREETGYKTLDGYIYNCLKGAYSEMASANLNAAVQKA | 59 | GUUGCAGCGUGC | 181 | |
| 5047 | WKKYKNSKTQVLQGVMSLPSYKSDQPILIDKGNVKLSAEE | | GCGAGCGUGUGG | | |
| | NNGRAVLTLFSRNYRDTRGLKGNVEFSVLLHDGTQKSIFR | | CUUGAAGG | | |
| | NLIDKTYALGQCQLVYERKKWFLLLTYSFTPAGHALDPEK | | | | |
| | ILGVDLGECYALYASSCYAPGILKIEGGEIAEYALRLEKR | | | | |
| | KRSLQQQARYCGEGRIGHGTKTRVGVVYKAEDRIASFRET | | | | |
| | INHRYSKELVDYAVSNGYGTIQMEDLSAIQKDLGFPKRLR | | | | |
| | HWTYYDLQMKITNKAKEHGIAVVKIDPRYTSQRCSKCGHI | | | | |
| | DPANRPRQEEFCCTACGYACNADYNASQNISIKGIEKIIQ | | | | |
| | KMLSAKAD | | | | |
| CasM.29 | MAEKTIVKVMKFELRYIDGAGEFSEMQKHLWELQKQTREV | 60 | GUUGCAAUUUGU | 182 | |
| 9588 | LNKTIQMGYALECKRFAHHDKTGQWLDDKELTGSKYKAVA | | AUACGAGUGUGA | | |
| | DYINAELKEDYNIFYSDCRNSTVRKAYKKFKDAKNKIFSG | | CUUGAAGG | | |
| | EMSLPSYRSNQPIIIHNRNVIIRGNAESALVGLKVFSDGF | | | | |
| | KALHGFPAAVNFKLCVKDGTQRAIIENVISEIYKISESQL | | | | |
| | IYDNKKWFLILAYRFTQKKNDLNPDKILGVDLGVKFAVYA | | | | |
| | SSIGEYGSFRIKGGEVTEFIKRLEKRKKSLQNQATVCGDG | | | | |
| | RIGHGTKTRVADVYKARDKISNFQDTINHRYSRAIVDYAR | | | | |
| | KNGYGTIQLEKLDNSIEKKGDYSPVLVHWTYYDLRTKMEY | | | | |
| | KAAEYGIKVIAVEPKYTSQRCSKCGYISSENRKTQESFEC | | | | |
| | IKCGYKCNADFNASQNLSVRDIDRIIDEYLGANPELT | | | | |
| CasM.27 | VVNVAKGALSKVMKFELSYLDGCGDFQNMQKELWTLQRQT | 61 | GCUGCAACACGC | 183 | YTTR; TTYN |
| 7328 | REILNRTIQIAYHWDYTDREHFKKTGQHLDVKSETGYKRL | | GCGGGUACGCGG | | |
| | DGYIYDELKETVQNFASVNVNATIQKAWAKYKSSKIDVLR | | GUUGAAGG | | |
| | GDMSLPSYKSDQPLVLHAQSIKLSEDKDGPVLQVTLESNA | | | | |
| | HKKACDYSNVRFAFRLHDATQRAIFKNVLSGEYGLGQSQI | | | | |
| | VYKRPKWFLYLTYNFSPEQHGLDPDKILGVDLGESIALYA | | | | |
| | SSLGDYGSLRIEGGEVTAFAKQLEARKRSLQKQAAHCGEG | | | | |
| | RVGHGTRARVSDVYKAEDKIANFRNTVNHRYSKKLIEYAI | | | | |
| | QNRYGTIQMEDLSGIKQDTGFPKFLQHWTYYDLQQKIEAK | | | | |
| | AKENGINFIKVDPSYTSQRCSKCGNIDSDNRPSQAVFCCT | | | | |
| | KCGFRANADFNASQNLSIPEIDKIIKKERGANTK | | | | |
| CasM.29 | MAKKGTNRKKMIVKVMKYELKYEKGCADFNEMQNELWKLQ | 62 | GUUGCAACUCGC | 184 | NCTT |
| 7894 | RQTREVMNRTVQLCYHWNYVQADYCKQHGCAHRDVKPCDV | | ACGUUGGCACUG | | |
| | YETNATSLDGYIYQLFKDEYPNFLMANLIATLRKAHQKYD | | AUUGAAGG | | |
| | ALLPDIQEGNSSIPSFKKDQPLIFSKEAIHLPECLSDKRQ | | | | |
| | ITLFCFSKPYKSAHPTLDKITFAVRAHSASEKSIFDNIIN | | | | |
| | GKYALGTSQLVYEKKKWFFLLSYKFTPESVDVNPEKVLGV | | | | |
| | DLGVVNALCAGSVENPHDSLFIKGTEAIEQIRRLEARKRD | | | | |
| | LQKQARYPGDGRIGHGTKTRVSPVYQTRDAIARMQDTLNH | | | | |
| | RWSRALIDFACKKGYGTIQMEDLSGIKAMESEKPYLKHWT | | | | |
| | YFDLQSKIIYKAEEKGIRVVKVNPKCTSRRCSACGYISKE | | | | |
| | NRKNQAEFLCVNCGYHHNADYNAAQNLSIPQIDRLIEKQL | | | | |
| | KEQESEESEAGANPK | | | | |
| CasM.29 | MTERHDNESSKIKAEVSLLNSSVPDFEKKRHVKVLKLHIL | 63 | GCUGUAGCCCUG | 185; | NNCC |
| 1449 | KPAGDMKWDELGALLRDARYRVFRLANLAISEAYLDFHKW | | CUCAAAUUGUAG | 186; | |
| | RSGGNEQPKLKISQLNRNLRSMLEDEVTGKQTKMIKSDRY | | GGCGCAUGCAGG; | 187 | |
| | SKSGALPDSIVSPLSMYKLGGLTSKSKWSEVLRGKSSLPT | | GUUGUAGUCGAC | | |
| | FKLNMAIPVRCDKPGDRRIERTKNGDAEVELRICLQPYPR | | CUGAAUCUGUGG | | |
| | VIIATGRNSLGDGQRAILDRLLDNTKYSEQGYRQRCFEIK | | GGUGCUUACAGG; | | |
| | EDQRSGKWHLFVTYDFPAIEPAKNLSRERIVGVDLGAACP | | UGUGGGGUGCUU | | |
| | LYAAINTGHARLGWKHFSPLAARVRALQNQTIRRRRQILR | | ACAGG | | |
| | GGKVSLSEDSARSGHGRKRKLKPISKLEGKIDRAYTTLNH | | | | |
| | QLSATVIKFAKDNGAGVVQMEDLKGLRETLIGTFLGERWR | | | | |
| | YEELQRFIRYKADEAGIEIRLVNPQYTSRRCSECGHIHKD | | | | |
| | FTREFRDKSREGNKSVRFLCPDCGFTADPDYNAARNLASL | | | | |
| | DIAAIIERQLEIQGLRKHDP | | | | |
| CasM.29 | MKEKSKTLVKVARLRILKPAGDMKWSELGEMLRTVRYRVF | 64 | GUUGUAGUCGAC | 188 | NNCC |
| 7599 | RLANLAVSEAYLGFHMYRTNRATEFKAETIGKLSRRLREM | | CUGAAUCUGUGG | | |
| | LIEEGVDEKDLSRYSQTGAVPDTVAGALGQYKIRGITSPT | | GGUGCUUACAGG | | |
| | KWRQVVRGQAALPTFRNDMAIPIRCDKQYQRRLEKTEAGE | | | | |
| | IEVELMICRKPYPRIVLGTADLGPGQRAILERLLQNTDNS | | | | |
| | ADGYRQRLFEAKQDTQTKKWWLYVTYDFPRLKEGKLNQEI | | | | |
| | VVGVDLGFSIPLYVALNIGHARLGRRHFQALGNRIRSLQR | | | | |
| | QVLARRRSIQRGGRVNISHSTARSGHGRKRKLLPTEKLRG | | | | |
| | RIEKSYSTLNHQLSASVIDFAKNHHAGTIQIEDLANLKEE | | | | |
| | LAGTFIGARWRYHQLQQFLKYKAEEAGITLNQVNPRYTSR | | | | |
| | RCSECGFINIDFDRAFRDAGRTEGRVTKFLCPECGYEADP | | | | |
| | DYNAARNISILDIDKLIRVQCKKQGLTYDAH | | | | |
| CasM.28 | MPERPKTVNKVIWFQIHKPAGDMTWKELGNLLREARYRVF | 65 | GGUGUAUGUAAC | 189; | NNCC |
| 6588 | RLANLAVSEKYLSFHMWRIGQEYKSETIGKLNRRLREMLI | | CGCAAUUUGAAG | 190; | |
| | EEGVEEESQKRFSATGALPDTVVSTLAKGKLAAITSKSKW | | GGUGCAUACAGG; | 191 | |
| | KDVVNGKTSLPTFKLNMAIPVRCDKAEQRRLRRTESGDVE | | GUUGUAGUCGAC | | |
| | LELMICKQPYPRVVLKTGKLKSGQRAILDRIVENNDNSKE | | CUGAAUCUGUGG | | |
| | GYSQRVFEIKQVENNDGSKEWRLYISYTFPKKAVEANADV | | GGUGCUUACAGG; | | |
| | AVGVDIGFSVPLVAAVNNGLERLGYNDFRALNERIRSLQR | | UGUGGGGUGCUU | | |
| | QVLVRRRSMQSGGRDYVSTPTARSGHGRKRKLLPIQTLRK | | ACAGG | | |
| | RWDNAYTTLNHQLSHAVVSFAENHGAATIQIENVKSLKDE | | | | |
| | LRGTFLGQRWRYFELQQFLKYKADEVGIELREVNARYTSR | | | | |
| | RCSECGYINMAFTRQARDKGRVDGKPMEFVCPECGYKAHP | | | | |
| | DYNAARNIAMLDIEQKMQVQCKQQGITYADDSEVL | | | | |
| CasM.28 | MTWPELGNMLRTVRYRVFRLANLAVSEAYLGFHMFRTKRA | 66 | GUUGGAAUCGAC | 192 | |
| 6910 | EEFKAETMGKLSRRLREMLIEEGVDEKDLSRYSQTGAVPD | | CUUAAUUUGAGG | | |
| | TVAGALSQYKIRGITSPTKWRQIVRGQVALPTFRNTMSIP | | UGUGCUUACA | | |
| | VRCDKLYQRRLEQGDSGEVEVELMICRNPYPRVVLGTGDL | | | | |
| | NPGQQAILERLLQNTDNSADGYRQRLFEIKEDVQTRKWWL | | | | |
| | YVTYDFPKTTGKLNPEIVVGVDLGFSIPLYVALNSGHARL | | | | |
| | GYLHFKALGERIKSLQKQVMARRRAIQRGGRVSISHSTAR | | | | |
| | TGHGVKRKLQPTEKLRGRIEKSYSTLNHQLSASVIDFAKN | | | | |
| | HHAGVIQIEDLSGLKEQLTGTFIGARWRYHQLQQFLKYKA | | | | |
| | EEAGITLKQINPRYTSRRCSECGFINMDFDRAFRDAGRTY | | | | |
| | GKVTKFLCPECGYEADPDYNAARNIATLDIEKLIRVQCEK | | | | |
| | HGLKFDAH | | | | |
| CasM.29 | VGKEGKRNVKVMKIRILKPCDGMTWNELGQLLRDARYRVF | 67 | GCUGAAAGAGCA | 193 | NNCC |
| 2335 | RLANLTVSEAYLNFHLWRTGRSQEFKKQTIGQLNRQLRNI | | GAGAAUUUGUUG | | |
| | LQQEKYDDEKLNRYSKTGALPDTVCSALWQYKLMAVMKKS | | UGUGCAUACAGG | | |
| | KWSEVIRGKSSLPTFRNDMAIPVRCDKPEQKRIEKTEQGQ | | | | |
| | VEAALQVCVQPYPRVILGTHTLGDGQDAILKRLLDNQNQA | | | | |
| | IGGYRQRSFEIKYDEQKRWWLFITYDFPATEVATDKTIAV | | | | |
| | GVDLGVSVPLYAAVNNGPARLGRREFGGLGRRIRDLRNQT | | | | |
| | DARRRSIQRSGREGQSDDTARAGHGRKRKLLPIHILEGRL | | | | |
| | DKAYTTLNHQMSAAVIKFAAEQGAGIIQIENLAGLQDELR | | | | |
| | GTFIGGRWRYRQLQDFLKYKTQEMGIELRQVNPKYTSRRC | | | | |
| | SKCGFIHKDFDRDYRNRHSENGKPAQFVCPNPDCKYESDP | | | | |
| | DYNAARNLATLDIEEQIRVQCQKQGLEYDSKKDKNAL | | | | |
| CasM.29 | MKEKSKTLVKVARLRILKPAGDMTWSELGEMLRTVRYRVF | 68 | GUUGGAGUCGGC | 194; | NNCC |
| 3576 | RLANLAVSEAYLGFHMFRTQRAAEFKAETMGKLSRRLREM | | UUGAAUCUGCGG | 195 | |
| | LIEEGVDEKELNCYSLTGAVPDTVAGALHQYKIRGITSPT | | GGUGCUUACAGG; | | |
| | KWRQVVRGQAALPTFRNDMSIPIRCDKPYQRRLEKTEAGE | | UGUGGGGUGCUU | | |
| | VEVELMICRKPYPRIVLGTADVGPGQEVILERLLQNKDNS | | ACAGG | | |
| | SDGYRQRLFEAKQDRQTGKWWLYVTYDFPRPEEGELNPEI | | | | |
| | VVGVDLGFSVPLYVAINNGYARLGRRHFQALGNRIRSLQR | | | | |
| | QVLARRRSIQRGGRVNISHDTARSGHGIKRKLLPTEKLRG | | | | |
| | RIEKSYSTLNHQLSASVIDFTKNHHAGTIQIEDLANLKEV | | | | |
| | LAGTFIGARWRYHQLQQFLKYKADEAGITLKEVNPRYTSR | | | | |
| | RCSECGFIHKDFDRAFRDSGRTDGKVARFVCPECGYGPVD | | | | |
| | PDYNAAKNISTLDIEKHIRVQCKKQGLEYEVH | | | | |
| CasM.29 | MKEKAKTLVKVARLRILKPAGDMTWPELGNMLRTVRYRVF | 69 | GUUGGAAUCGAC | 196 | NNCC |
| 4537 | RLANLAVSEAYLGFHMFRTKRAEEFKAETMGKLSRRLREM | | CUUAAUUUGAGG | | |
| | LIEEGVDEKDLSRYSQTGAVPDTVAGALSQYKIRGITSPT | | UGUGCUUACAGG | | |
| | KWRQIVRGQVALPTFRNTMSIPVRCDKLYQRRLEQGDSGE | | | | |
| | VEVELMICRNPYPRVVLGTGDLNPGQQAILERLLQNTDNS | | | | |
| | ADGYRQRLFEIKEDVQTRKWWLYVTYDFPKTTGKLNPEIV | | | | |
| | VGVDLGFSIPLYVALNSGHARLGYLHFKALGERIKSLQKQ | | | | |
| | VMARRRAIQRGGRVSISHSTARTGHGVKRKLQPTEKLRGR | | | | |
| | IEKSYSTLNHQLSASVIDFAKNHHAGVIQIEDLSGLKEQL | | | | |
| | TGTFIGARWRYHQLQQFLKYKAEEAGITLKQINPRYTSRR | | | | |
| | CSECGFINMDFDRAFRDAGRTYGKVTKFLCPECGYEADPD | | | | |
| | YNAARNIATLDIEKLIRVQCEKHGLKFDAH | | | | |
| CasM.29 | MAKKAKTMFKVTNFRILKPAGDMTWKELGQLLRDARYRTF | 70 | GUUGUAAGAGAC | 197 | NNTC |
| 8538 | RMANLALSEAYLNFYLLKKGDLKEYKNVKIGQIAKRLRDM | | CCGAAUUUUAGC | | |
| | LIEEGVDEEVQNRESPKVALPAYVYSALDQFKLRGLTSKS | | UGUGUAUACAGG | | |
| | NWKKVLRGQASLPTFRLNMSVPIRCDKPEHRRLEKTENGN | | | | |
| | VEVDLMICRKPYPRVVLETLKLDGSSKAILDRLLENEDNS | | | | |
| | PGNYRQRCFEVKQNPRSNDWWLYVTYEMPVDKDKKLDPKV | | | | |
| | IVGVDLGFSVPLYVAINNGHARLGRRHFQALGKRIHNLQN | | | | |
| | QVLARRRSIQRGGQVNLSHSTSRSGHGRKRKLQPTEKLQQ | | | | |
| | KINSAYSTLNHQLSSSVIDFANNHKAGTIQIEDLETLKEQ | | | | |
| | LTGTYIGRQWRYYQLQQFIEYKAKENSITVKKINPKYTSR | | | | |
| | RCSMCGHIHADFDRTFRDRSSNKGFVTKFICPECNFEADP | | | | |
| | DYNAAKNISTLDIENKIKLQCKKQKIDY | | | | |
| CasM.19 | MPKITRKIELLFDRSGLSEEECKEKWRFIYQINDNLYRVA | 71 | GUUGUGAAUGCA | 198; | NTCG |
| 924 | NRLVNQLYLADEIDDILRLSDQEYIALRKKLANKKLDEAT | | GGCAUUUUUGAU | 199 | |
| | RISLEEQMSQVMKRVNERRSAILQRPQQSFAYSVVTDSDT | | GGUAAAUCCAAC; | | |
| | EGLTAKILDVLKQDVLSHYKADTKEVLKGEKSISNYKKGM | | UGGUACAUCCAA | | |
| | PIPFAFNDSLRLYKEDGFFYLKWYNGIRFLLNFGRDASNN | | C | | |
| | QLIVERCLGISKDEISYKACSSSIQIKKKGNHSKIFLLLV | | | | |
| | VDVPVEQYAQKPNMVVGVDLGLNVPIYAASNSTLERKAIG | | | | |
| | SREAFLNQRGAFQRRFRALQRLQTTKGGRGRLHKLEPLER | | | | |
| | VREAERNWVRTQNHLFSREVINFAIDVGASTIQMEKLANF | | | | |
| | GRDAQGEVREDKKYVLRNWSYFELQNLIEYKAKRAGIKVK | | | | |
| | YINPAFTSQTCSECGQLGERDSIHFKCTNPDCPNCGKDIH | | | | |
| | ADYNGARNIAKSKDYIK | | | | |
| CasM.19 | MPTITRKIELTLLTEGLSEEQRKEQWGLLYHINDNLYKAA | 72 | ACUGUCAGACAA | 200; | NTCG; NKYG |
| 952 | NNISSKLYLDDHVSSMVRMKHAEYLSLLKELARAEKQKTP | | UGCAAAAUGUGU | 201; | |
| | DADAIAELRKKVAAAEKEMTDQEHAICKYATEMSTQSLSY | | GGUACAUCCAAC; | 202; | |
| | RFATELETNIFAKILDCLKQGVFATFNSDARDVKRGERAI | | UGGUACAUCCAA | 203; | |
| | RNYKKGMPIPFAWDKSLRIEKDNKDFYLRWYNGLRFLFNF | | C; | 204; | |
| | GKDRSNNRLIVERCLKMDADYDGEYKLCNSSIQIAKREGK | | UGGUACAUCC; | 205; | |
| | TKLFLLLVVKIPQEHVELNKKVVVGVDLGINVPAYVATNI | | UGGGGCAGUUGG | 206; | |
| | TEERKAIGDREHFLNSRMAFQRRYKSLQRLRGTAGGKGRA | | UUGCCCUUAGCC | 207; | |
| | KKLEPLERLRKAEHNWVHTQNHLFSREVVDFAVKSHAATI | | UGAGGCAUUUAU | 208; | |
| | HMEDLSGFGKDNDGNADERKEFVLRNWSYYELQNMIAYKA | | UGCACUCGGGAA | 209; | |
| | AKYGIKVEKIHPAYTSKTCSWCGQLGFREGVTFICENPEC | | GUACCAUUACAU | 210; | |
| | KQCGEKVHADYNAARNIANSKDIIKKNE | | CCAAC; | 211; | |
| | | | UGGUACAUCCAA | 212; | |
| | | | CUCUAGGCGCC; | 213; | |
| | | | AAUGGUACAUCC | 214; | |
| | | | AAC; | 215; | |
| | | | UGGUACAUCCAA | 216; | |
| | | | CUCUAGGC; | 217; | |
| | | | UGGUACAUCCAA | 218; | |
| | | | CUCUAGGCGC; | 219 | |
| | | | UGGUACAUCCAA | | |
| | | | CUCUAGGCG; | | |
| | | | UGGUACAUCCAA | | |
| | | | CUCUAGG; | | |
| | | | AAAUGGUACAUC | | |
| | | | CAAC; | | |
| | | | UGGUACAUCCAA | | |
| | | | CUCU; | | |
| | | | UGGUACAUCCAA | | |
| | | | CUC; | | |
| | | | UGGUACAUCCAA | | |
| | | | CU; | | |
| | | | UGGUACAUCCAA | | |
| | | | CUCUAG; | | |
| | | | UGGUAUAUCCAA | | |
| | | | C; | | |
| | | | UGGUACAUCCAA | | |
| | | | CUCUA; | | |
| | | | AUGGUACAUCCA | | |
| | | | AC; | | |
| | | | UGGUACAUCCAA; | | |
| | | | UGGUACAUCCA | | |
| CasM.27 | MPTITRKIELTLCTDGLSEEQRKEQWGLLYHINDNLYKAA | 73 | GCUGUCAGUAGU | 220; | NTCG |
| 4559 | NNISSKLYLDDHVSSMVRMKHAEYLSLLKELARAEKQKTP | | AGUAAAAAUGGG | 221 | |
| | DADAIAELKKKVAATEKEMTDQEHAICKYATEMSTQSLSY | | GGUACAUCCAAC; | | |
| | RFSTEFETKIFAKILDCLKQGVFATFNSDAKDVKRGERAI | | UGGUACAUCCAA | | |
| | RNYKKGMPIPFAWTDSLRIKKDNKDFYLLWYNGLRFLFNF | | C | | |
| | GKDRSNNRLIVERCLKMDADYDGEYKLCNSSIQIAKREGK | | | | |
| | VKLFLLLVVSIPKEHVELNKKVVVSVDLGINVPAYVATNI | | | | |
| | TEERKAIGDREHFLNSRMAFQRRYKSLQRLKGTTGGKGRT | | | | |
| | KKLEPLERLRKAEHNWVHTQNHLFSREVVDFAVKTHAATI | | | | |
| | HMEDLSGFGKDNDGNADERKEFVLRNWSYYELQNMISYKA | | | | |
| | AKYGIKVEKIRPAYTSKTCSWCGQHGFREGVTFICENPAC | | | | |
| | KQCGEKVHADYNAARNIANSKEIIKKNE | | | | |
| CasM.28 | MPTITRKIELTLCTEGLSDQERKDQWNLLYHINDNLYRAA | 74 | ACUGUCAGUACA | 222; | RTTR |
| 6251 | NNISSKLYLDDHVGSMVRLKHAEYLSLLRALEKAKKQKAP | | UGCAAAAAUGAG | 223 | |
| | DEEVIAELSQQVATAEQEMDEQAKAICQYATEMSTQTLSY | | GGUACAUCCAAC; | | |
| | RFATELETNIFGQILTCLRQGVFSTENSDARDVKRGERSI | | UGGUACAUCCAA | | |
| | RTYKKGMPIPFPWNDSLRIGFEDGEFYLRWYNGLRFRFDF | | C | | |
| | GKDRSNNCLIVQRCMKMDKDYEGDYKLCNSSIQMVKREGK | | | | |
| | PKFFLLLVVNIPQERVELNKNIVVGVDLGINAPAYVATNT | | | | |
| | TPERKQIGDREHFLNERMAFQRRFKSLQRLKGTTGGRGRA | | | | |
| | KKLEPLERLRKAEQNWVHTQNHLFSREVIDFAVKARAATI | | | | |
| | HMEDLSGFGKDNDGNADERKEFVLRNWSYYELQNMITYKA | | | | |
| | AKYGIKVEKIRPAYTSKTCSWCGHQGFREGITFICENPEC | | | | |
| | KKFGEKEHADYNAARNIANSKEIIKNNEE | | | | |
| CasM.28 | MPTITRKIELTLLTEGLSEEQRKEQWGLLYHINDNLYKAA | 75 | ACUGUCAGACAA | 224; | NTCG |
| 8480 | NNISSKLYLDDHVSTMVRMKHAEYLSLLRELARAEKQKKP | | UGCAAAAUGAGU | 225 | |
| | DVDAIAELREKVTAAEKEMSDQERAICTYATEMSTQSLSY | | GGUACAUCCAAC; | | |
| | RFATEIETNIFAKILDCLKQGVFATFNSDARDVKRGERAI | | UGGUACAUCCAA | | |
| | RNYKKGMPIPFAWDKSLRIEKDNKDFYLRWYNGLRFLFNF | | C | | |
| | GKDRSNNRLIVERCLKMDADYDGEYKLCNSSIQIVKREGK | | | | |
| | VKLFLLLVVSIPQEHVELNKKIVVGVDLGINVPAYVATNI | | | | |
| | TEERKAIGDREHFLNSRMAFQRRYKSLQRLKGTAGGKGRT | | | | |
| | KKLEPLERLRKAEHNWVHTQNHLFSREVVDFAVKSHAATI | | | | |
| | HMEDLSGFGKDNDGNADERKEFVLRNWSYYELQNMIAYKA | | | | |
| | AKYGIKVERIRPAYTSKTCSWCGQLGFREGVTFICENPEC | | | | |
| | KQCGEKVHADYNAARNIANSKDIIKKNE | | | | |
| CasM.28 | MPTMTRKIELKLCTEGLSDEERKAQLGLLYHINDNLYKAA | 76 | GCUGUUAGAACA | 226 | |
| 8668 | NNISSKLYLDDHVSSMVRLKHAEYLSLLNEFEKAKKKGDE | | UACAAAAUGAAA | | |
| | EQIVELSLRVAAAEKELTDQELAICKYATEMSTDTLAYRF | | GGUACAUCCAAC | | |
| | ANEIEINVFGQILACLKQGIHSTFKKDAADVKRGERAIRN | | | | |
| | FKKGMPIPFPWSKSIRIENEGSDFYLRWYNGLRFRFDFGK | | | | |
| | DRSNNRLIVSRCLNLDPDFEDEYKLSNSSLQMVKRDGRPK | | | | |
| | LFLLLVVNIPQENVELNKKIVVGVDLGINSPAYVATNITM | | | | |
| | ERQRIGSRDTFLNARMAIQRRFQSLQKLQNTAGGRGRKKK | | | | |
| | LEPLERLKETERNWVRTQNHLFSRDVVQFAVKTRAATIHM | | | | |
| | EDLSGFGKDDDGNADEKKEFVLRNWSYYELQTMIKYKAAK | | | | |
| | YGIKVEKIRPAYTSRTCSWCGHEGDRKGETFICENPECEK | | | | |
| | YGKKENADYNAARNIANSTDIIK | | | | |
| CasM.28 | MPTITRKIELTLCTEGLSDEQRKEQWGLLYHINDNLYKAA | 77 | GCUGCAUGUCAU | 227; | RTTR |
| 9206 | NNISSKLYLDEHVSSMVRMKHAEYLSLLKELARAEKQQTP | | GGCAAAAGGAAA | 228 | |
| | DEGLIAELSRKLSAAEKEMADQELAICKYATEMSTQTLSY | | GGUACAUCCAAC; | | |
| | NFAKEIETNIFGQILTCLRQGVYATFNSDAKDVKRGERAI | | UGGUACAUCCAA | | |
| | RNYKKGMPIPFPWNNSLKIESDSGEFYLRWYNGLRFLLTF | | C | | |
| | GKDRSNNRMIVNRCMKMDEDFEGEYKLCNSSIQLAKRDGK | | | | |
| | PKLFLLLVVNIPQEHVKLNKKIVVGVDLGVNVPAYVATNI | | | | |
| | TEERKAIGDREHFLNTRMAFQRRYKSLQRLKGTAGGKGRT | | | | |
| | KKLEPLERLRDAERNWVHTQNHLFSREVVNFAVQARAATI | | | | |
| | HMEDLSGFGKDKDGNADEKKEFVLRNWSFYELQNMIAYKS | | | | |
| | AKYGIKVVKIRPAYTSKTCSWCGQQGDRKSTTFICENPKC | | | | |
| | KHYGESIHADYNAARNIANSNDIVKENE | | | | |
| CasM.29 | MPKITRKIEMTLCTEGLSDEQRKEQWGLLYHINDNLYKAA | 78 | GCUGUCAGACAC | 229; | RTTG; RTTR |
| 0598 | NNISTKLYLDEHVSSMVRMKHADYLSLLKELAKAEKKSPD | | CUAAAAAAUGAG | 230 | |
| | EDLIAELREKLAAAEQEMTDQELAICKYATEMSTQTLAYK | | GGUACAUCCAAC; | | |
| | FATEIEINVFGQILACLKQAAQSNFKSDAKDVKRGERAIR | | UGGUACAUCCAA | | |
| | NYKKGMPIPFPWNDNIRIDADGDEFYLRWYNGLRFHLTFG | | C | | |
| | KDKSNNRMIVKRCLKMDKDFEGEYKLCNSSIQMVKRDGKP | | | | |
| | KLFLLLVVNIPQEHVELNKNVVVGVDLGVNVPAYVATNIT | | | | |
| | EERKAIGEREHFLNTRMQIQRRYKSLQRLKATAGGKGRTK | | | | |
| | KLEPLERLRKAEHNWVHTQNHLFSREVVNFAVQTHAATIH | | | | |
| | MEDLSGFGKDDDGNADEQKEFVLRNWSFYELQNMIAYKAA | | | | |
| | KYGIKVEKVKPAYTSKTCSWCGQLGFRQGVTFICENPACK | | | | |
| | QCGEKVHADYNAARNIANSKDIIKKNE | | | | |
| CasM.29 | MPTITRKIELHLCTDGLIDEQQKAQRLLLYHINDNLYKAA | 79 | GCUGUGAGUCAC | 231; | NTCG; RTTR |
| 0816 | NNVSSKLYLDEHVSSMVRLKHDEYLSLSRELARAEKKHDD | | AGUAAAAAUGAA | 232 | |
| | ELTTELRGKLAAAEREMTDQELAICKYATEMSTQSLSYRL | | GGUAUAUCCAAC | | |
| | VTELETKIFAKILDCLKQGVYATFNSDARDVKRGERAIRN | | UGGUACAUCCAA | | |
| | YKKGMPIPFAWNDSVRIEYDEKEKDFYLRWYNDIRFKFHF | | C | | |
| | GRDRSNNRLIVSRCLKLDKDYEGDYQLCNSSIQIVKRDGS | | | | |
| | TKFFLLLVVKIPQEHVELNKRIVVGVDLGINYPAYVATNC | | | | |
| | TEERMYIGDREHFLNTRMQFQRRYKSLQKLKGTAGGKGRS | | | | |
| | KKLEPLERLRNAERNWVHTQNHLFSLKVVNFAVQTHAATI | | | | |
| | HLEDLSGFGKDDDGNADERKEFVLRNWSYYELQSMIEYKA | | | | |
| | KKYGIKVEKIRPAYTSQTCSWCGQRGFRQGVTFICENPEC | | | | |
| | KKCGEKENADYNAARNIANSKDVIKDKNE | | | | |
| CasM.29 | TPFVLYFQNYSLSLRQHITLYSMPTITRKIELTLCTEGLS | 80 | ACUGUCAGUACA | 233; | RTTR |
| 5071 | DQERKDQWNLLYHINDNLYRAANNISSKLYLDDHVGSMVR | | UGCAAAAAUGAG | 234 | |
| | LKHAEYLSLLRAMEKAKKQKAPDEEVIAELSQQVAAAEQE | | GGUACAUCCAAC; | | |
| | MDEQAKAICQYATEMSTQTLSYRFATELETNIFGQILTCL | | UGGUACAUCCAA | | |
| | RQGVFSTENSDARDVKRGERSIRTYKKGMPIPFPWNDSLR | | C | | |
| | IGFEDGEFYLRWYNGLRFRFDFGKDRSNNRLIVQRCMKMD | | | | |
| | KDYEGDYKLCNSSIQMVKREGKPKFFLLLVVNIPQERVEL | | | | |
| | NKNIVVGVDLGINAPAYVATNTTPERKQIGDREHFLNERM | | | | |
| | AFQRRFKSLQRLKGTTGGRGRAKKLEPLERLRKAEQNWVH | | | | |
| | TQNHLFSREVIDFAVKARAATIHMEDLSGFGKDRDGNADE | | | | |
| | RKEFVLRNWSYYELQNMITYKAAKYGIKVEKIRPAYTSKT | | | | |
| | CSWCGHQGFREGITFICENPECKKFGEKEHADYNAARNIA | | | | |
| | NSKEIIKNNEE | | | | |
| CasM.29 | MPTITRKIELHLCTEELSDEQQKAQRLLLYHINDNLYKAA | 81 | GCUGUGAGUCAC | 235; | NTCG; NKCG |
| 5231 | NNVSSKLYLDEHVSSMVRLKHDEYLSLLRELARAEKKADD | | AGUAAAAAUGAA | 236 | |
| | ELATQLREKLVAAEREMTDQELAICKYATEMSTQSLSYRF | | GGUAUAUCCAAC; | | |
| | VTELETKIFAKILDCLKQGVYATFNSDSRDVKRGERAIRN | | UGGUACAUCCAA | | |
| | YKKGMPIPFAWDKSVRIEYEEKEKDFFLRWYNDIRFKFHF | | C | | |
| | GRDRSNNRLIVSRCMKLDKDYEGDYQLCNSSIQIVKRDGS | | | | |
| | TKYFLLLVVKIPQEHVELNKKIVVGVDLGINYPAFAATNC | | | | |
| | TEERMSIGDREHFLNTRMQFQRRFKSLQRLKGTTGGKGRN | | | | |
| | KKLEPLERLRKAEHNWVHTQNHLFSLKVVNFAVQAHAATI | | | | |
| | HLEDLSGFGKDDDGNADERKEFVLRNWSYYELQNMIKYKA | | | | |
| | KKFGIQVEKIRPAYTSQTCSWCGQRGFRQGITFICENPEC | | | | |
| | KKCGEKENADYNAARNIANSKDIIKDKDE | | | | |
| CasM.29 | MPIITRKIELHISKEGLSAEDYKAQWQYLRQINDNLYMAA | 82 | GAUGUAUAUGCU | 237; | KNTK; KNTT |
| 2139 | NRVSSHCFLNDEYKYRLCLQIPDYIDIEKQLKDSKRARLS | | AUGAUUUUGUAU | 238 | |
| | KEELGQLKKRKKELENTVKGRFQDEFEKNSLYTIISNEFG | | GGUACAUCCAAC; | | |
| | EIIPGQILTCLRQCVQSKYNRAKEELEKGERAISTYKKGM | | UGGUACAUCCAA | | |
| | PIPFPINKSIRLQKQGEDFVLKWYNKIVFKLHFGRDRSNN | | C | | |
| | RVIVERLIQSALNDKQKGEDYVMNNSSIQLVEKDKMTKIF | | | | |
| | LLLSMDIPTQKRKLDSELVLGVDLGLNFPLYYATNQSANI | | | | |
| | HDHIGDKDIFLKERMVFQRRFKELQRLQCTQGGRGRKKKL | | | | |
| | EPLEKLRDKERNWVRTKNHIFSREVIKVALHLGAGTIHLE | | | | |
| | NLHNFGKDGNGELKNSKKFVFRNWSYFELQSMIEYKAKME | | | | |
| | GITVKYVNPAYTSQTCSVCGMIGERKEQAVFRCMNSSCLE | | | | |
| | YGKEVNADFNAARNIAKAKM | | | | |
| CasM.27 | MPTITRKIELTLCTDGLSDDLRKDQWQLLYHINDNLYKAA | 83 | GCUGUCAGUAGU | 239; | RTTR |
| 9423 | NNISSKLYLDEHVASMVRLKHAEYLGLIKELAKARKRADD | | AGUAAAAAUGGG | 240; | |
| | EAVRDLCSKLAVAEQEMNEQAKAICDYATEMSTQTLSYNF | | GGUACAUCCAAC; | 241 | |
| | AKEIETNIFGQILTCLRQGVLLNENSDARDVKRGERAIRN | | GUUGCAGAACCC | | |
| | YKKGMPIPFPWNDTIKIVSEGDEFYLRWFSGLRFHLNFGK | | GAAUAGACGAAU | | |
| | DRSNNRMIVRRCLKMEQDFDEEYKISNSSIQVAKRDGKQK | | GAAGGAAUGCAA | | |
| | LFLLLVVQIPQEQVVLNKKIVVGVDLGVNVPAYVATNITE | | C; | | |
| | ERKAIGDREHFLNTRMQFQRRYKSLQRLKTTEGGRGRAKK | | UGGUACAUCCAA | | |
| | LEPLERLRKAEHNWVHTQNHLFSREVVNFALQTQAATINM | | C | | |
| | EDLSGFGKDNDGNADECKEFVLRNWSYYELQNMIVYKASK | | | | |
| | YGIRVQKIRPAYTSKTCSWCGHMGFREGVTFICENPDCKQ | | | | |
| | FGEKVHADYNAARNIANSKEIIKNDE | | | | |
| CasM.20 | MSKTVTKTVKIALICEHTNKYGEKVDYKDINKLLWKLQKQ | 84 | GUUGAGCUCUGC | 242; | NTTC; NYTC |
| 054 | TRELKNKTIQLCWEYNNFSCDYYKEHHEYPNMEDILKYKR | | AUUACGCAGAUG | 243; | |
| | INGFVENKLKTVNDLYSSNCSTTILSTCNEFQNYRSEFLK | | AAUGACGA; | 244 | |
| | GTRSINSYKSDQPLDLHKGAIKLEHDGKDFYVSLKLLKRS | | GAUAUAUCUUGU | | |
| | AFNAMEFKGSDIRFKLNVKDKDKSTLKILESCYDKIYSIS | | AUGCAUAUGUAG | | |
| | ASKMTYDRKAGKWFLLLAYSFTPAKTENLDPEKILGVDLG | | GUUGUGAG; | | |
| | IKIPICASVYGDLDRLTIEGGKIEEFRRRVEARKRSLQKQ | | GUUGCAACUUAC | | |
| | GKQCGDGRIGHGTKKRIKPITDIGDKIARFRDTENHIYSR | | GCAUAGGUGUAA | | |
| | YLIEYAVKKGCGTIQMEKLEGITREKDIFLKNWTYFDLQK | | AAUACGAGG | | |
| | KIEYKAKEKGIKVVYIEPAYTSKRCSSCGFIDIDNRLDQA | | | | |
| | HFKCLKCGFNENADYNASQNIGIKNIDKIIKEEHKSASDK | | | | |
| | LTSE | | | | |
| CasM.28 | VIILTKVVKLYLISEQINKEGQKIDYQRINSILWDLQKQT | 85 | GAUGCAACUUAG | 245; | NTTC; TTCN |
| 2673 | RDIKNRTVQLCWEWMNFSSDYCKTQEEYPKERDILGYTLE | | AUGCAUAUGUAA | 246; | |
| | GYVYDYFKTGYDLYTGNISTSSREVCSSFKNVKKEILKGE | | GUUGUGAG; | 247; | |
| | RSILSYKANQPLDLHKKAISLEYDNFNFFVKLKLLNRTGK | | GUUGCAAUGAAC | 248; | |
| | KKYDITEDINFKIQVNDKSTRTILERCYDKEYKISGSKLI | | GUAUGUGCAUGA | 249; | |
| | YEKKKKLWRLNLCYSFENSQVETLEKDKILGIDLGIVYPL | | GGUGUGAG; | 250 | |
| | MASIYGEYDRFSIKGGEIEEFRRRTEARKRSILQQTKYCG | | GAUAUAUCUUGU | | |
| | DGRIGHGRNKRTQPAYKINDKIARFRDTANHKYSRALIEY | | AUGCAUAUGUAG | | |
| | AVKKNCGIIQMENLTGISDNTDCFLKDWSYYDLQTKIENK | | GUUGUGAG; | | |
| | AKEMGIKVVYIKAQYTSQRCSRCGYIDVNNRIRQALFKCQ | | GUUGCAACUUAC | | |
| | NCGYETNADYNASQNIGMYDIENIIEETLKIQSANVKQS | | GCAUAGGUGUAA | | |
| | | | AAUACGAGG; | | |
| | | | GUUGCAAUUCGU | | |
| | | | AUGCGCAGGUAA | | |
| | | | GUUUCGAG; | | |
| | | | UGUGCAUGAGGU | | |
| | | | GUGAG | | |
| CasM.28 | MTKVTKVYLISEQIDKDGNKIDFKKISELLWNLQMQTRDI | 86 | GUUGCAAUCUGC | 251; | NTTY; NNTY |
| 2952 | KNKCVQLCWEWLNFSSDYYKKSEEYPKEKDTLGYTLSGFV | | GUACAGGCGUAA | 252; | |
| | YDRIKNGSDLYSSNLSTSSRDTCTAFSNYKKEMLKGERSV | | GAUGUGAG; | 253; | |
| | LSFKANQPLDIHNKAIKLSYENGNFFVALKMLNRAGKEKY | | GUUGCAAUGAAC | 254 | |
| | GIKDDLRFRMQVRDKSVRTILERLMNDEYKVSASKLMYDK | | GUAUGUGCAUGA | | |
| | KKKLWKLNLCYSFDNHVISTLDTEKIMGVDLGVVYPIMAS | | GGUGUGAG; | | |
| | VNGDYARFSIKGGEIEAFRSRVEARRRSLLNQSRYCGDGR | | UGUGCAUGAGGU | | |
| | IGHGRKKRTEPATQIADKIARFRDTTNHKYSRALIDYAIK | | GUGAG; | | |
| | NGCGTIQMEKLTGITSSAEHFLKEWSYFDLQTKIESKAKE | | CAGGCGUAAGAU | | |
| | AGIKVVYINPKFTSQRCNKCGYIHTDNRPVQARFCCQKCG | | GUGAG | | |
| | YEENADYNASQNIGTKHIDVIIEETLKMQCEPETPTE | | | | |
| CasM.28 | MNKVVKLALICEQSDKDNSPVDYKKINEILWELQKQTREI | 87 | GAUCAUAUCUGC | 255; | NTTC; TTCN |
| 3262 | KNKAIQYCWEYNNFSSDYYKKFNEYPKEKDILSYTLVGFV | | UUGUAUGGGUAU | 256; | |
| | NDKFKTGNDLYSGNCSTTVRNACTEFKNSKKELIKGSRSI | | GCUGCGAG; | 257; | |
| | INYRSNQPLDIHNKCIRIEFENNCFYTYLKLLNRPAFKKY | | GAUAUAUCUUGU | 258; | |
| | NFANTEIKFKILVRDNSTKTILERCISNEYEIAASKLLYD | | AUGCAUAUGUAG | 259; | |
| | QKKKCWFLNLVYAFEIKSNNSLDPNKILGVDLGIHYPICA | | GUUGUGAG; | 260; | |
| | SVYGSLDRFTIDGGEIDEFRRRVESRKISMLKQGKNCGDG | | GUUGCAAUGAAC | 261 | |
| | RIGHGIKARNKPVYNIEDKIARFRDTANHKYSRALIEYAV | | GUAUGUGCAUGA | | |
| | KHTCGTIQMEDLTGITDIANRFLKNWSYYDLQTKIEYKAK | | GGUGUGAG; | | |
| | EAGINIVYIDPKNTSRRCSKCGYIDKENRETQSRFICLKC | | GUUGCAACUUAC | | |
| | GFKENADYNASQNIGIKDIDKLIKEDVH | | GCAUAGGUGUAA | | |
| | | | AAUACGAGG; | | |
| | | | GUUGCAAUUCGU | | |
| | | | AUGCGCAGGUAA | | |
| | | | GUUUCGAG; | | |
| | | | UGUGCAUGAGGU | | |
| | | | GUGAG; | | |
| | | | UAUGGGUAUGCU | | |
| | | | GCGAG | | |
| CasM.28 | VTLLVKVVKIYLISEQFDKAGNQIDYKEVNKILWELQKQT | 88 | GUUGCAACUUAC | 262; | TYYT; NNYN; |
| 4833 | REAKNKTVQLLWEWNNFSSDYVKASGIYPKAKDIFGYSSV | | GCAUAGGUGUAA | 263 | CTTN; NNNT |
| | HGQANKELRTKLALNSSNLSTTTMDVCKIFNTYKKEVWEG | | AAUACGAG; | | |
| | KRSVPSYKSDQPLDLHKESIKLIYENNEFYVRLALLKKAE | | GAUAUAUCUUGU | | |
| | FAKYGFKDGFRFKMQVKDNSTKTILERCFDEVYKINASKL | | AUGCAUAUGUAG | | |
| | LYDQKKKKWKLNLSYSFDNKNISELDKEKILGVDVGVNCP | | GUUGUGAG | | |
| | LVASVFGDRDRFIIKGGEIEKFRKSVEARRRSMLEQTKYC | | | | |
| | GDGRIGHGRKKRTEPALNIGDKIARFRDTINHKYSRALIE | | | | |
| | YAVKKGCGTIQMEKLTGITSKSDRFLKDWTYYDLQTKIEN | | | | |
| | KAKEVGINVVYIAPKYTSQRCSKCGYIHKDNRPNQAKFRC | | | | |
| | LECDFESNADYNASQNIGIKNIDKIIEKDLQKQESEVQVN | | | | |
| | ENK | | | | |
| CasM.28 | MNKVVKLALICEQSDKNNSPVDYKKVNEILWELQKQTREI | 89 | GAUUAUAUCUGC | 264; | NTTC |
| 7700 | KNKTIQYCWEYYNFSSDYYKKENKYPKEKDILSYTLWGFI | | UUGUAUGGGUAU | 265 | |
| | NDKFKTGNDLYSGNCSATTKKVIKEFKNSKKELIRGSRSI | | ACUGCGAG; | | |
| | INYKSNQPLNIHNKCIHLQFKNNNFYVSINLLNRRSFKKY | | GUUGCAACUUAC | | |
| | NFANTAIKFKILVRDNSTKAILERCISNEYKISESQLIYN | | GCAUAGGUGUAA | | |
| | KKKKCWFLNLSYAFEIKSNNSLDPNKILGVDLGIHYPICA | | AAUACGAGG | | |
| | SVYGSLDRFTIDGGEIDEFRRRVESRKISMLKQGKNCGDG | | | | |
| | RIGHGIKARNKPVYNIEDKIARFRDTANHKYSRALIEYAV | | | | |
| | KNNCGTIQMEDLTGITDNANRFLKNWSYYDLQTKIEYKAK | | | | |
| | EASINVVYINPENTSRRCSKCGYIDKENRKTQSSFICLKC | | | | |
| | GFKENADYNASQNISIKDIDKLIKEDVH | | | | |
| CasM.29 | VTLLVKVVKIHLISEQFDKAGNRIDYEEVNKILWELQKQT | 90 | GUUGCAACUUAC | 266; | NNYT; WNCT |
| 1507 | REAKNKTVQLLWEWNNFSSDYVKASGIYPKAKDIFGYSSV | | GCAUAGGUGUAA | 267; | |
| | HGQANKELRTKLALNSSNLSTTTMDVCKNFNTYKKEVWKG | | AAUACGAG; | 268; | |
| | KRSVPSYKSDQPLDLHKDSIKLIYENNQFYVRLALLKKAE | | GUUGCAAUGAAC | 269 | |
| | FAKYGFKDGFHFKMQVKDNSTKTILERCFDEVYKINASKL | | GUAUGUGCAUGA | | |
| | LYDQKKKKWKLNLSYSFDNKNISELDKEKILGVDVGVSYP | | GGUGUGAG; | | |
| | LVASVFGDRDRFKIKGGEIEKFRKSVEARRRSMLEQTKYC | | GAUAUAUCUUGU | | |
| | GDGRIGHGRKKRTEPALNIGDKIARFRDTTNHKYSRALIE | | AUGCAUAUGUAG | | |
| | YAVKKGCGTIQMEKLTGITSKADRFLKDWTYYDLQTKIEN | | GUUGUGAG; | | |
| | KAKEVGINVVYIAPKYTSQRCSKCGYIHKDNRPNQAKFRC | | GUUGCAACUUAC | | |
| | LECDFESNADYNASQNIGIKNIDKIIEKDLQKQESEVQVN | | GCAUAGGUGUAA | | |
| | ENK | | AAUACGAGG | | |
| CasM.29 | LIWKDALGGIILTKIVKLYLISEQIDKDGNRVDYKEINSI | 91 | UCAGCUCACAAC | 270; | NTTC; TTYN; |
| 3410 | LWNLQKQTRDIKNKTVQLCWEWMNFSSDYYKKNELYPNEK | | CUACAUAUGCAU | 271; | TYYW |
| | EILNLTLRGYAYDHFKQGYDLYSSNISVLTEAVCGAFKNA | | ACAAGAUAUAUC | 272; | |
| | KKEMLNGEKSVLSYKAEQPLDIHKKCIKLEYDKNFYVKLK | | GU; | 273; | |
| | MLNKAGKKKYGIEDDLNFKIQVEDKSTRTILERCIDGEYV | | GUUGCAAUGAAC | 274; | |
| | VSGSKLIYDKKKKLWKLNLCYSFKANEIESLDKNKILGID | | GUAUGUGCAUGA | 275; | |
| | LGIACPLMASVNGEFDRFSIKGGEIETFRKRIEARKRSVL | | GGUGUGAG; | | |
| | HQTKYCGDGRIGHGRNKRTEPAYKINDKIARFRDTANHKY | | GAUAUAUCUUGU | | |
| | SRALIDYAIRKNCGMIQMENLTGISDKKEHFLKEWSYYDL | | AUGCAUAUGUAG | | |
| | QTKIENKAKEKGIKIVYINPEYTSQRCSKCGYIDANNREL | | GUUGUGAG; | | |
| | RAVFKCQKCGFEADADYNASQNIGIKNIEDIIENTLKISS | | GUUGCAACUUAC | | |
| | ANEKQTKNT | | GCAUAGGUGUAA | | |
| | | | AAUACGAGG; | | |
| | | | GUUGCAAUUCGU | | |
| | | | AUGCGCAGGUAA | | |
| | | | GUUUCGAG; | | |
| | | | UGUGCAUGAGGU | | |
| | | | GUGAG | | |
| CasM.29 | VFYSTFLCYILTKYIDFSANECYNINTSSEVKQLMNKVVK | 92 | GAUCAUAUCUGC | 276; | NTTC |
| 5105 | LALICEQSDKDNSPVDYKKINEILWELQKQTREIKNKAIQ | | UUGUAUGGGUAU | 277; | |
| | YCWEYNNFSSDYYKKFNEYPKEKDILSYTLVGFVNDKFKT | | GCUGCGAG; | 278; | |
| | GNDLYSGNCSTTVRNACTEFKNSKKELIKGSRSIINYRSN | | GAUAUAUCUUGU | 279; | |
| | QPLDIHNKCIRIEFENNCFYTYLKLLNRPAFKKYNFANTE | | AUGCAUAUGUAG | 280; | |
| | IKFKILVRDNSTKTILERCISNEYEIAASKLLYDQKKKCW | | GUUGUGAG; | 281; | |
| | FLNLVYAFEIKSNNSLDPNKILGVDLGIHYPICASVYGSL | | GUUGCAAUGAAC | | |
| | DRFTIDGGEIDEFRRRVESRKISMLKQGKNCGDGRIGHGI | | GUAUGUGCAUGA | | |
| | KARNKPVYNIEDKIARFRDTANHKYSRALIEYAVKHTCGT | | GGUGUGAG; | | |
| | IQMEDLTGITDIANRFLKNWSYYDLQTKIEYKAKEAGINI | | GUUGCAACUUAC | | |
| | VYIDPKNTSRRCSKCGYIDKENRETQSRFICLKCGFKENA | | GCAUAGGUGUAA | | |
| | DYNASQNIGIKDIDKLIKEDVH | | AAUACGAGG; | | |
| | | | GUUGCAAUUCGU | | |
| | | | AUGCGCAGGUAA | | |
| | | | GUUUCGAG; | | |
| | | | UGUGCAUGAGGU | | |
| | | | GUGAG | | |
| CasM.29 | LISEQIDKDGNRVDYKEINSILWNLQKQTRDIKNKTVQLC | 93 | GAUAUAUCUUGU | 282; | NTTY |
| 5187 | WEWMNFSSDYYKKNELYPNEKEILNLTLRGYAYDHFKQGY | | AUGCAUAUGUAG | 283; | |
| | DLYSSNISVLTEAVCGAFKNAKKEMLNGEKSVLSYKAEQP | | GUUGUGAG; | 284; | |
| | LDIHKKCIKLEYDKNFYVKLKMLNKAGKKKYGIEDDLNEK | | CAUAUGUAGGUU | | |
| | IQVEDKSTRTILERCIDGEYVVSGSKLIYDKKKKLWKLNL | | GUGAG; | | |
| | CYSFKANEIESLDKNKILGIDLGIACPLMASVNGEFDRES | | UGUAGGUUGUGA | | |
| | IKGGEIETFRKRIEARKRSVLHQTKYCGDGRIGHGRNKRT | | G | | |
| | EPAYKINDKIARFRDTANHKYSRALIDYAIRKNCGMIQME | | | | |
| | NLTGISDNKEHFLKEWSYYDLQTKIENKAKEKGIKIVYIN | | | | |
| | PEYTSQRCSKCGYIDANNRELRAVFKCQNCGFEADADYNA | | | | |
| | SQNIGIKNIEDIIENTLKISSANEKQTKNT | | | | |
| CasM.29 | LVKVVKIYLISEQVDEQGKDVDYNTICGVLWDLQWETREI | 94 | GUUGCAAUGAAC | 285; | NTTY; NNTY; |
| 5929 | KNKTVQLCWEWSGFSSDYYKKYGEYPKEKNLLDYTMGGFV | | GUAUGUGCAUGA | 286; | NTTC |
| | YDKLKSKYHLYTANLSTTSQNTCGIFRTYKVDFVKGNRSV | | GGUGUGAG; | 287 | |
| | LSFKADQPLDVHKKSISIDRIDDNYFVKLKLINKSGIQKY | | UGUGCAUGAGGU | | |
| | GIRDDFHFRMLVKDNSTKTILERCVGGDYKAAASKIIYDK | | GUGAG; | | |
| | KKKMWCLNLSYEFDVNTAKDLNKNRILGIDIGIVYPVVAS | | CAUGAGGUGUGA | | |
| | VNGELDRFVIQGGEIETFRRRVENRKKSLLKQTKYCGDGR | | G | | |
| | IGHGRNKRTEPVDIISDQIARFRNTANHKYSRAVIDYAVR | | | | |
| | KQCGTIQMENLKGITDKSDRFLKNWSYYDLQQKIEYKAKE | | | | |
| | KGINVVFINPKYTSQRCSRCGYIDSANRPKLPNQSKFLCI | | | | |
| | KCGFTENADYNASQNIALYNIEKLIDAEA | | | | |
| CasM.19 | LHETEKSLKFAEKYIAMPTITRKIELTLCTEGLSDEQRKE | 95 | AGGUACAUCCAA | 288; | RTTN; NTCG; |
| 498 | QWGLLYHINDNLYKAANNISSKLYLDEHVSSMVRMKHAEY | | C; | 289 | RTTR; KRTTN |
| | LSLQKELARAEKQKVDDAIIVELTRKLAVAEKEMTDQELA | | UGGUACAUCCAA | | |
| | ICKYATEMSTNTLAYNFAKEIETKIFGQILACLENNAHAL | | C | | |
| | FVDDSPNVRRGERAIRNYKKGMPIPFPWNRSIKIEADGGE | | | | |
| | FYLRWYNGLRFLLTFGKDRSNNRLIVKRCMKMDEVFEGEY | | | | |
| | KLCNSSIQLAKRDGKPKLFLLLVVNIPQEHVELNKNIVVG | | | | |
| | VDLGVNVPAYVATNITEERKAIGDREHFLNTRMTFQKRYK | | | | |
| | SLQRLKGTAGGKGRTKKLEPLERLRDAERNWVHTQNHLFS | | | | |
| | REVVNFAVQARAATINIEDLSGFGKDNDGNADEKKEFVLR | | | | |
| | NWSYYELQNMITYKASKYGIKVEKIRPDYTSKTCSWCGQQ | | | | |
| | GFREGVTFICENPECKQHGEKIHADYNAARNIANSKDIIK | | | | |
| | KNE | | | | |
| CasM.19 | MAETKRLQKVAKFQIVKPVNMSWDELGRMLRDVRYRLSRL | 96 | UGCGGUGUAAUU | 290 | NCCN; NCCR |
| 548 | ANMAVSETYQNLHQRYRLKNQDAPKSLKIGQLSRNLRKIL | | CGAGG | | |
| | REEGVEEENLSKYSKTCVLPDTITGAFSRYKLSSIDWRKV | | | | |
| | LTGKISVPNYKTNLSIPIRCDKPHQRRLELTETGEIEADL | | | | |
| | MICNKPYPRVLLSTRTISDGQRTVLERLVSNKTNFLPGYR | | | | |
| | HRFFEVKEKKGKWELSVTYDFPKAEATRLHPDIIVGVDLG | | | | |
| | WSVPLYAAINNGYARIGYRKFEPLAKRIKHLQKQIKGRRF | | | | |
| | STQKGGVKDLAQPTARAGHGRKRILKPIEKLEYKIDNAYT | | | | |
| | TLNHQLSHCVVEFAKNNGAGLIQIENLEGLKDDLSGTFIG | | | | |
| | QNWRYNQLQNFIKYKADEAGIKVHPVNPCYTSRRCSHCGF | | | | |
| | IHISFDREYRDKNRKNGKATMFECPKGCKPLNADYNAAKN | | | | |
| | LATFDIEEKIRLQCKQQSIEYKELPKD | | | | |
| CasM.19 | MPGTEKRLQKVATFEIVKPVNMSWPEFGKMLRDVRYRYWR | 97 | GAUGUGAACGAC | 291 | TTYN; |
| 910 | LANMAVCENYMRFYQWRTQQTDANDRYKVKILNRILRKML | | CUUUUUUUGCGG | | WTTYN |
| | IEEKNADEKELSRYSRDGAVSGYICGAFEKTKLSAVKSSS | | UGUGCUUCGAGG | | |
| | KWKKVIAGKESLPLFKKDLAIPINCSDHQPRLIERTQSGE | | | | |
| | YEVDLRICQQPYPRVLLSTAKISDGQKAILERLVSNETNS | | | | |
| | LPGYRHRFFEIKEKRNKWYLSVSYDFPKIDATRLHPNIIV | | | | |
| | GVDLGWSVPLYAAISNGYARIGYRKLKALGDRIKALQRQT | | | | |
| | IARRRSIQRTGEQDLSAPTARSGHGRKRILHPIEKLEGKI | | | | |
| | DNAYKTLNHQLSHCVIEFAKNHGAGLIQVENLKGLAEELS | | | | |
| | GTFIGQNWRYNQLQEFIKYKAKEAGIEVKEVNPCYTSRRC | | | | |
| | SECGFIHKEFTFEYRQANKKTDKATMFECPKCGYKAIADY | | | | |
| | NAARNLANPDIAEKIRLQCKEQGIEYKELPKD | | | | |
| CasM.19 | MPTITRKIELHFCTEGLSDEKQKEQRQLLYHINDNLYKAA | 98 | AGGUACAUCCAA | 292 | RTTN |
| 948 | NNISSKLYLDEHVSSMVRLKHADYLSLQRELARAEKQKTP | | C | | |
| | DDELITELSRKLSAAEKEMTDQELAICKYATEMATSTLAY | | | | |
| | NFAKEMETEIFGQILACLENNAHAVFVDDSLSVKRGERAI | | | | |
| | RNYKKGMPIPFPWNKNIKIETKDCEFYLRWYNGIRFRLHF | | | | |
| | GKDRSNNRLIVQRCLKLDDNFESEYKLCNSSIQLDKRDGK | | | | |
| | TKLFLLLVVNIPQEHVELNKNIVVGVDLGLNYPAYVATNS | | | | |
| | TEERKYIGDRDHFLKIRMQFQSRYKSLQRLKGTAGGKGRA | | | | |
| | KKLEPLERLRKAERNWVHTQNHLFSRDVVNFAVQTHAATI | | | | |
| | HMEDLSGFGKDNDGNADEKKEFVLRNWSYYELQSMIEYKA | | | | |
| | AKYGIKVEKIRPAYTSKTCSWCGQQGDRKSTTFICENPEC | | | | |
| | KHYGESIHADYNAARNIANSKDIVKKNE | | | | |
| CasM.26 | MSKITRKIEIIPDIDGITHEESNKKCYNTFYKFDRKLYKV | 99 | GGUGGAUAUCAU | 293; | NNTG; TNTG; |
| 5291 | ANLLVSQLYGLDNLLSLMRLQNDEYVKCQSKLSFKSITDA | | CUUAAAAAGUGA | 294 | NNNG |
| | TKEEIKKRMQEIDAELVSMKNDIAPKHPQTYSYRAVTSSE | | GGUACAUCCAAC; | | |
| | YAKDIPSDILNNLKQDVYQHFNENKKEQIRGERSLATYKK | | GGUGGAUAUCAU | | |
| | GMPIPFSFEKRHVIICDGDNYYLPWFEDTRFRLNFGRDRS | | CUUAAAAAGUGA | | |
| | NNRAIIDNCIKTKKYKLCAAAKIQLKERKLFLLITVDIPK | | GGUACAUCCAAC | | |
| | AESVPVKGKVMGVDLGVINPAYVAVNDGPERSRIGNGEAF | | | | |
| | QKQRDVFRRRFRELQRSQLTQGGHGRKHKTKATEILRGKE | | | | |
| | RNWVQTENHRISREIVNLASRWKVETIQMESLKGFGKNQE | | | | |
| | GEVEYNHKRLLGRWSYFELQKDIEYKAAMAGIAVQYVNPA | | | | |
| | YTSQTCHVCGQRGNRIERDTFICTNPECTCYNQAQDADMN | | | | |
| | AAINIAKSKDVIK | | | | |
| CasM.27 | MPTITRKIELTLCTDGLSDEERKAQWGLLYHINDNLYKAA | 100 | UGGUACAUCCAA | 295 | NRTT; ANRTT |
| 0012 | NNISSKLYLDEHVSSMVRLKHAEYLSLQKELAKAERQKMP | | C | | |
| | DVDVIEELRERLSAAEQEMSDQELAICKYATEMSINTLAY | | | | |
| | RFATEIETNIFGQILARLENNAQAVFLTDAPDVKRGERAI | | | | |
| | RNYKKGMPIPFPWNNSIKIECEGGEFYLRWYSGLRFHFNF | | | | |
| | GKDRSGNRLIVQRCLKLDKEYDGEYKLCNSSIQMVKRDGS | | | | |
| | TKFFLLMVVNIPQEYVELNKHIVVGVDLGINVPAYVATNI | | | | |
| | TPERKAIGDREHFLNTRMAFQRRYKSLQRLKTTAGGKGRT | | | | |
| | KKLEPLERLRQAEHNWVHTQNHLFSREVVNFALQTHAATI | | | | |
| | HLEDLSGFGKDSDGNADERKEFVLRNWSYYELQNMITYKA | | | | |
| | AKYGIRVEKIRPAFTSRTCSCCGHEGFREGVTFICENPEC | | | | |
| | QQFGEKVHADYNAARNIANSKDIIKKNE | | | | |
| CasM.27 | MPTITRKIELTLCTEGLSEEQRKEQWGLLYHINDNLYKAA | 101 | UGGUACAUCCAA | 296; | RTTR; RTWG |
| 2451 | NNISSKLYLDDHVSSMVRMKHAEYLSLLKELARAEKQKTP | | C; | 297 | |
| | DADAIAELRKKVAAAEKEMTDQEHAICKYATEMSTETLAY | | UGGUACAUCCAA | | |
| | KFATEIETNVFGQILACLKQAAQSNFKNDAKDVKRGERAI | | C | | |
| | RNYKKGMPIPFPWNDSLRIEKDNKDFYLRWYNGLRFLENF | | | | |
| | GKDRSNNRLIVERCLKMDADYDGEYKLCNSSIQIAKREGK | | | | |
| | VKLFLLLVVSIPQEHVELNKKVVVGVDLGINVPAYVAINI | | | | |
| | TEERKAIGDREHFLNTRMAFQRRYKSLQRLKGTAGGKGRT | | | | |
| | KKLEPLERLRKAEHNWVHTQNHLFSREVVDFAVKTHAATI | | | | |
| | HMEDLSGFGKDNDGNADERKEFVLRNWSFYELQNMITYKA | | | | |
| | AKYGIKVEKIRPAYTSKTCSCCGRQGFRSGVTFICENPEC | | | | |
| | KQYGEKVHADYNAARNIANSKEIIKKNE | | | | |
| CasM.27 | MKNNVEEKRPDKEKRLTKVATFQIVKPVNMSWSEFGKMLR | 102 | GGUGUGAACGAC | 298; | NCCN |
| 4429 | DVRYRLSRLANMAVSEAYQNLHQRYRLKNQNAPKSVKIGQ | | CUUUUUUUGCGG | 299; | |
| | ISRDLRKILLEEGLEEENLSKYSKMCVLPDTITGAFSRYK | | UGUAAUUCGAGG; | 300 | |
| | LSTIDWRKVLIGKISIPNYKANLSIPIRCDKPQQRRLERT | | UGCGGUGUAAUU | | |
| | ETGEIEVDLMICNKPYPRVLLSTRTISDGQRSVLERLVLN | | CGAGG; | | |
| | NANSLPGYRHRIFEIKEKRNEWYLSVTYDFPKAETTKLHS | | UUGCGGUGUACU | | |
| | DIIVGVDLGWSVPLYAAINNGYARIGYKQLKPLGDSIKAL | | UCGAGG | | |
| | QRQTIARRRSIQRGGTQDLAAPTARSGHGIKRILQPIEKL | | | | |
| | EGKIDNAYKTLNHQLSHCVIEFAKNHGAGVIQIENLKGLA | | | | |
| | EELSGTFIGQNWRYYQLQEFIKYKAKEAGIIVKEVNPFYT | | | | |
| | SRRCSECGYIHKDFTFEYRQANRKNGKSTMFECPKKEEKG | | | | |
| | CKPLNADYNAARNLATSDIEDKIRLQCKEQGIEYKEIKEK | | | | |
| CasM.27 | VTLLVKVVKIHLISEQFDKAGNRIDYKEVNKILWELQKQT | 103 | GUUGCAACUUAC | 301; | TTYN, NNYN, |
| 7378 | REAKNKTVQLLWEWNNFSSDYVKASGIYPKAKDIFGYSSV | | GCAUAGGUGUAA | 302 | YTTR |
| | HGQANKELRTKLILNSSNLSTTTMDVCKIFNTYKKEVWEG | | AAUACGAGG; | | |
| | KRSVPSYKSDQPLDLHNDSIKLIYENKEFYVRLGLLNRAG | | GUUGCAACUUAC | | |
| | FAKYGFKDGFRFKMQVKDNSTKTILERCFDGIYTIVASKL | | GCAUAGGUGUAA | | |
| | LYDQKKNRWKLNLSYSFDNKNISELDKEKILGVDVGVSCP | | AAUACGAGG | | |
| | LVASVFGDRDRFIIKGGEIEKFRKSVEARRRSMLEQTKYC | | | | |
| | GDGRIGHGRKKRTEPALNIGDKIARFRDTTNHKYSRALIE | | | | |
| | YAVKKGCGTIQMEKLTGITSKADRFLKDWTYYDLQTKIEN | | | | |
| | KAKEVGINVVYIAPKYTSQRCSKCGYIHKDNRPNQAKFRC | | | | |
| | LKCDFESNADYNASQNIGIKNIDKTIKKERKKQKSEAQVN | | | | |
| | EK | | | | |
| CasM.28 | MAGKKKDKDVINKTLSVRIIRPRYSDDIEKEISDEKAKRK | 104 | GUUGCAGAACCC | 303; | |
| 0852 | QDGKTGELDRAFFSELKSRNPDIITNDELFPLFTEIQKNL | | GAAUAGACGAAU | 304 | |
| | TEIYNKSISLLYMKLIVEEEGGSTASALSAGPYKECKARF | | GAAGGAAUGCAA | | |
| | NSYISLGLRQKIQSNFRRKELKGFQVSLPTAKSDRFPIPF | | C; | | |
| | CHQVENGKGGFKVYETGDDFIFEVPLIKYTATNKKSTSGK | | AGAAGAAGGAUU | | |
| | NYTKVQLNNPPVPMNVPLLLSTMRRRQTKKGMQWNKDEGT | | GGGAC | | |
| | NAELRRVMSGEYKVSYAEIIRRTRFGKHDDWFVNFSIKFK | | | | |
| | NKTDELNQNVRGGIDIGVSNPLVCAVTINGLDRYIVANNDI | | | | |
| | MAFNERAMARRRTLLRKNRFKRSGHGAKNKLEPITVLTEK | | | | |
| | NERFRKSILQRWAREVAEFFKRTSASVVNMEDLSGITERE | | | | |
| | DFFSTKLRTTWNYRLMQTTIENKLKEYGIAVNYISPKYTS | | | | |
| | QTCHSCGKRNDYFTFSYRSENNYPPFECKECNKVKCNADE | | | | |
| | NAAKNIALKVVL | | | | |
| CasM.28 | MPDTDKGKRLTKVATFQIVKPVNMSWNEFGKMLHDVRYRY | 105 | GAUAUAUCUUGU | 305 | TTTN |
| 1050 | WRLANMAVCENYMRFYRWRTQQTDTNDHYKVKIINGILRK | | AUGCAUAUGUAG | | |
| | MLIEEKNADEKELSRYSRDGAVSGYVYGAFTQTKLSAITS | | GUUGUGAG | | |
| | KSKWGEVIKGKSALPLFKRDTSIPIMCTDKKPSMIEKTAS | | AAUGUGAACGAC | | |
| | GEYEVDLRICLKDKQLRPNGYPSVLLSTTKISDGQKAVLE | | CUUCUUUUGCGG | | |
| | RLVSNKTNSLPGYRHRFFEVKEKRGDWYLSVSYDFPQAEA | | UGUACUUCGAGG | | |
| | TRLHPDIIVGVDLGWSVPLYAAINNGYARIGWRKLEPLAK | | | | |
| | SIKHLQKQTIVRRRSFQKGGKKDLAASTARTGHGIKRILQ | | | | |
| | PIEKLEGKIDNAYKTLNHQLSHCIIEFAKNHGAGVIQIEN | | | | |
| | LKGLAEELSGTFIGQNWRYHQLQEFIKYKAEEAGIAVKEV | | | | |
| | NPRYTSRRCSKCGYIHIGFDREYRDKNRKNGKSTMFECPE | | | | |
| | CSKRIKDYKPLNADYNAAKNLATADIEEKIRLQCKEQGIE | | | | |
| | YKELPKD | | | | |
| CasM.28 | MPTITRKIKLELCTKGLSEEERKAQWNLLYHINDNLYRSA | 106 | AGGUACAUCCAA | 306 | GTYG, |
| 5333 | NNISSKLYLDEHVSSLVWLKHKEHQTLKADLAKAKKQKIQ | | C | | RGTYG |
| | DEKTIAELESRLKSCESEMSDQELAICKYTDEMSSKTLSY | | | | |
| | KFATELELNIYAQILTQVQSKVYADFQNDQKDVRDGKRAI | | | | |
| | RTYKKGMPIPFPWRNNIRMEPVKKGREYEFYIKWYNDIRF | | | | |
| | QLIFGKDRSNNRLILQRCFKLDENCVEDYQMRTSSIKMVK | | | | |
| | GANGTELFLYLVVDIPQEKHILNNKIVVGVDLGINVPAYV | | | | |
| | ATNVTDDRKAIGDREHFLNTRMAISKRFHSFQRLKGTTGG | | | | |
| | RGKTKKLEPLERLKEKERNWVHTQNHLFSRDVITFALHVK | | | | |
| | AATIQMEDLSGYGKDDEGNVVEEKKFLLGKWSYYELQEMI | | | | |
| | KYKAKKVGMRVNFIKPAYSSQTCSWCGERGERNSTSFVCT | | | | |
| | NSECSHYGEDLHADYNAARNIARSKNIIRYE | | | | |
| CasM.28 | MIITRKIQILFAAQGEEFKKDKDTLYKWSNIVHHASNIVA | 107 | AAGGUUGAUACA | 307 | |
| 6285 | SNKYVCDHLQGMVYLTEEGKEAVSELSQKVDDIENTSRMN | | GC | | |
| | TTYRMISSLYKGEIPTDILSCVNMQVSKLYNKERKKMADG | | | | |
| | DRSLRSYRSNIPIPFSANSLMRKWKYADKEYSFDLFGIPF | | | | |
| | KVVLGKDKSNNRSILERLMDGTYKAATSSIKIQNCEDETG | | | | |
| | KKTRKFFLLLCVEIPDKSYAGREDNILFAELSIDHPLLVS | | | | |
| | FPIKKEESKPIPIGNKQSYLYKRLQIQKGLDSCKASCKWN | | | | |
| | KGGRGRKRKMKSTERFKAKEHNFVDAYMHQISAALIKFAI | | | | |
| | KHDIGKLCLVDVDKKIKEAKESPFVLRNWSYYSLLTKIQY | | | | |
| | KAKMNGITVVMVDKNVL | | | | |
| CasM.28 | MPTITRKIRLHLCTDGLSEEERKAQWKMLYRINDNLYRAA | 108 | GCUGUAAGUCAU | 308; | RTRG |
| 6678 | NNISSKLYLDEHISSMVRLKHAEYTSLKTELLKAKKADDE | | GGAAAAAUGGUG | 309 | |
| | ETVAELEARINVLNAELSAQEEAICSYATEMATRTLAGKF | | AGUACAUCCAAC; | | |
| | ASELDLNIYGQILAEVKSVVFKNFNSDSKDVREGKRSIRT | | AUGGUGAGUACA | | |
| | YKKGMPIPFPWNKTIRLEAVKKESSSKHDEDEYEVYLNWY | | UCCAAC | | |
| | KSSRTEKKAIRFRLDFGKDKSNNQQIVKRCLNLDNTSSES | | | | |
| | YQLQTSSIQMKKGSEGAELYLLLVVNIPQDQHVLNKKIVV | | | | |
| | GVDLGINVPAYVATNCTEERKSIGDREHFLNARIAFHRRF | | | | |
| | HSFQKLKGTTGGRGRKKKLEPLERLREKERNWVHTQNHLI | | | | |
| | SRDVINFALQTKAATIQMEDLSGYGKDEEGNVKPENKELQ | | | | |
| | SRWSYFELQSMIKYKAAKCGIKVNLINPSYTSQTCSWCGQ | | | | |
| | MGVRESTSFVCQNPECKKYGKDIHADYNAARNIARSNKTV | | | | |
| | KNE | | | | |
| CasM.28 | MPTITRKIELRLCTEGLSDEERKAQWMLLYHINDNLYRSA | 109 | GAGCACAUCCAA | 310 | RTNG |
| 7128 | NNISSKLYLDEHVSSMVRLKHAEYQSTAAELLKAKKNNAD | | C | | |
| | EGTISTLEDKVETLKTEMSAQGIAICNYATEMATRTLAGK | | | | |
| | FASELELNIYGQILAEVKNVVHTNFTNDAKEVREGKRSIR | | | | |
| | TYKKGMPIPFPWNKSIKIEPVKASSQNEGQDDYEFYLKWY | | | | |
| | NGLKFILHFGKDRSNNRQILKRCFGLDNLCNERYQMRTSS | | | | |
| | IQMKKGSNGMELYLLLVLSIPKEQHSLNKKVVVGVDLGIN | | | | |
| | VPAYVATNCTEERRAIGDREQFLNTRMAIIRRKHSFQRLK | | | | |
| | GTAGGRGRKKKLDPLERLRETERNWVHTQNHLYSRDIIKF | | | | |
| | ALETKAATIQMEKLKGFGRDDNGNVIEEKKFLLGKWSYYE | | | | |
| | LQNMIKYKAGKVGIKVNFIAPAYTSQTCSCCGVRDDRNRK | | | | |
| | STSFICHNPDCQMYGKEIHADYNAARNIARSKNVIKDE | | | | |
| CasM.28 | MPAITRKIELTLCTEGLSEEQRKEQWGLLYHINDNLYKAA | 110 | GGGUACAUCCAA | 311; | RTTN |
| 7826 | NNISSKLYLDEHVSSMVRMKHADYLSLLKELARAEKQKTP | | C; | 312 | |
| | DDELIAELREKLSLAEQEMTDQELAICNYATEMATSTLAY | | UGGUACAUCCAA | | |
| | NFAKEIETEIFGQILACLENNAHAVFVDDSPTVRRGERAI | | C | | |
| | RNYKKGMPIPFPWNKSIRIVEKDGEFYLRWYNGMRFLLTF | | | | |
| | GKDRSNNRIIMKRCLKMDQDFEGEYKLCNSSIQMVKREGK | | | | |
| | TKLFLLIVVNIPQEHVELNKNIVVGVDLGVNVPAYVATNI | | | | |
| | TEERKAIGDREHFLNTRMQFQRRYKSLQRLKGTAGGKGRT | | | | |
| | KKLEPLERLRKAEHNWVHTQNHLFSREVVNFAVQTRAATI | | | | |
| | HMEDLSGFGKDNDGNADEQKEFVLRNWSFYELQNMIAYKA | | | | |
| | AKYGIKVEKVKPAYTSKTCSWCGQLGFRQGVTFICENPAC | | | | |
| | KQCGEKVHADYNAARNIANSKDIIKKNE | | | | |
| CasM.28 | MAGQRHTKVAKFQILKPAADMRWSELGRLLRDAQYRVYRL | 111 | GUUGCGUUUGCC | 313 | |
| 7896 | ANLALSEKYLRFHLFRTGQTESLPECRIGRLNRQLRQMLK | | CGUGAUUUCGGG | | |
| | DEGGADDSVLDRFSRTGALPDTVVGALWQYRLHALTKGEK | | UGUGUAUACAGG | | |
| | WNKVTRGETALPTFRRSMALPIRCDKRIHHRLERAALDSV | | | | |
| | ELDLMICTRPYPRVILKTAKLDDGAAAILERLLDNEGQLL | | | | |
| | EGYRQRCFEVRYAEDEKAWWLHVTYDSPATPAPHLSKDII | | | | |
| | VGVDLGFSCPMYVALSNGDARLGRRQFAALAARIRSLQTQ | | | | |
| | VMARRRQMLSGGKASLSGDTARSGHGRKRKLLPIESLEGR | | | | |
| | INRAYTTLNHQLSISVVHFAVHHGAGVIQIENLEGLQNEL | | | | |
| | TGTFLGQRWRYHQLQEFLNYKANEAGIEVRRVNPRYTSRR | | | | |
| | CSKCGYIHVDENRAFRDAARQEGKVARFCCPKCEYEAHPD | | | | |
| | YNAARNLATVDIEGIIKVQCERQGIDRPSVENQDEVAK | | | | |
| CasM.28 | MPTITRKIELTLCTEGLSDQERKDQWNLLYHINDNLYRAA | 112 | UGGUACAUCCAA | 314 | RTTR |
| 7936 | NNISSKLYLDDHVGSMVRLKHAEYLSLLRALEKAKKQKAP | | C | | |
| | DEEVIAELSQQVATAEQEMDEQAKAICQYATEMSTQTLSY | | | | |
| | RFATELETNIFGQILTCLRQGVFSTENSDARDVKRGERSI | | | | |
| | RTYKKGMPIPFPWNDSLRIGFEDGEFYLRWYNGLRFRFDF | | | | |
| | GKDRSNNRLIVQRCMKMDKDYEGDYKLCNSSIQMVKREGK | | | | |
| | PKFFLLLVVNIPQERVELNKNIVVGVDLGINAPAYVATNT | | | | |
| | TPERKQIGDREHFLNERMAFQRRFKSLQRLKSTTGGRGRA | | | | |
| | KKLEPLERLRKAEQNWVHTQNHLFSREVIDFAVKARAATI | | | | |
| | HMEDLSGFGKDRDGNADERKEFVLRNWSYYELQNMITYKA | | | | |
| | AKYGIKVEKIRPAYTSKTCSWCGHQGFREGITFICENPEC | | | | |
| | KKFGEKEHADYNAARNIANSKEIIKNNEE | | | | |
| CasM.28 | MPTITRKIELSLCTDGLSDEQLKEQRQLLYHINDNLYRAA | 113 | AGGUAUAUCCAA | 315 | NTCG; NKCG |
| 8450 | NNVSSKLYLDEHVSSMVRLKHADYLSLLRDLARAEKQKSP | | C | | |
| | DEALISELRSKLAAAQREMTEQELAICRYATEMSTQSLSY | | | | |
| | RFVTEMETHIFAKILDCLKQGVYATFNSDARDVKRGERSI | | | | |
| | RNYKKGMPIPFAWSDSVRIEQEADEFYLRWYNGIRFRLVF | | | | |
| | GKDRSNNRLIVKRCLKLDKDYEGDYKLCNSSVQMVKREGK | | | | |
| | PKTFLLLVVKIPQEQVELNKKIVLGVDLGINYPVYAATNC | | | | |
| | TEERIYFGEREHFLNTRMQFQRRYKSLQRLKGTTGGKGRK | | | | |
| | KKLEPLERLRKAERNWVHTQNHLFSQKTVDFALQTHAATI | | | | |
| | HLEDLSGFGRDSDGSAEEKKEFVLRNWSYYELQQMITYKA | | | | |
| | AKYGIKVEKIRPAYTSQTCSWCGQRGFRQGVTFICENPEC | | | | |
| | KKCGEKEQADYNAARNIAKSKDVIKDDDE | | | | |
| CasM.28 | MSIVTRKIELIPDIENLTHEESNQRCYKLLYNIDKKLYKL | 114 | GGCGUAUGUCUA | 316 | NNTG; WNTG |
| 8712 | ANLLVCQLFGLDNLLSLMRLQNDEYVKFQSKLASKSISKE | | CCUGAAAAAGAA | | |
| | TQKNIKEHMKEIDKELLARKAEIAPKSPLTFAYRAIKGSL | | GGUAUAUCCAAC | | |
| | YAKDLPSDIFNTLKQDVFKHFNETKKEQLRGERSLATYKR | | | | |
| | GIPIPFSLMKKNVIVSEGDNYYLTWFEETRFKLNFGKDRS | | | | |
| | NNRAIIDNCLKINKYKLCTAAKIQLKNKKLFFLVTVDIPE | | | | |
| | TKNTIIKGKVMGVDLGVVHPAYVAVNDGPERSLIGDGDAF | | | | |
| | QKQRDVFRRRFKELQRCQLTQGGHGRKHKTKATEILRGKE | | | | |
| | RNWVQTENHRISRKIVNLAIRWKVESIQMENLKGFGKDSE | | | | |
| | GEVETKHKRLLGRWSYFELQKDIEYKAQKAGIKVVYINPA | | | | |
| | YTSQTCHVCGKKGDRTERDTFICLNTECSCYGKPQDADMN | | | | |
| | AAINIARSKNIVK | | | | |
| CasM.28 | MPTITRKIELMLCSEGLSDEQRKEQWGLLYHINDNLYKAA | 115 | UGGUACAUCCAA | 317 | RTTR |
| 9248 | NNISSKLYLDEHVSSMVRMKHAEYLSLLKELARAEKQQTP | | C | | |
| | DEGLIAELSRKLSAAEKEMADQELAICKYATEMSTQTLSY | | | | |
| | NFAKEIETNIFGQILTCLRQGVYATFNSDAKDVKRGERAI | | | | |
| | RNYKKGMPIPFPWNKSLKIEAEGGDFYLRWYNGLRFLLTF | | | | |
| | GKDRSNNRMIVKRCMKMDEDFEGEYKLCNSSIQLAKRDGK | | | | |
| | PKLFLLLVVNIPQEHVELNKKIVVGVDLGVNVPAYVATNI | | | | |
| | TEERKAIGDREHFLNTRMAFQRRYKSLQRLKGTAGGKGRT | | | | |
| | KKLEPLERLRDAERNWVHTQNHLFSREVVNFAVQARAATI | | | | |
| | HMEDMSGFGKDKDGNADEKKEFVLRNWSFYELQNMIAYKS | | | | |
| | AKYGIKVVKIRPAYTSKTCSWCGQQGERKSTTFICENPEC | | | | |
| | KHYGESIHADYNAARNIANSNDIVKENE | | | | |
| CasM.28 | MVITRKIEVFVCESDNDLRRSYYEKLYDIRNIAQEAANRA | 116 | GGCUACAUACAG | 318 | |
| 9726 | TSMLYAIDNLIPCLDEDSRKLIQYIGAKGTPASRQNAAYT | | C | | |
| | IMSHLYKDRMPGIMDMLSNLAQYVTKNYSEDRKRGMYKNA | | | | |
| | LRSYKCSLPVPYQKKSFKGLRFNWYEDSDGDAHEGCFFSL | | | | |
| | AGVPLQMRFGRDRSNNRLIVERVISGEYKMCTSSLKFDGK | | | | |
| | KLFLLLCVDIPKQEANVDPKKTLYAYLGVMNPIICTCDVR | | | | |
| | AKQEYDSGYKCFEIGTKEEFNYRRRQIQEAVRRCQINNRY | | | | |
| | SSGGKGRKKKCQAIERWHEKEKNYVDTKLHTYSRMLVDLA | | | | |
| | VAHKCGTIVLLNQKKREDKAKDDNQNGEPFVLRNWSYYNL | | | | |
| | KDKIGYKCKLAGIKLVQDKEETEEE | | | | |
| CasM.28 | MVITRKIEVFVCEDSKDLRKEYYDKIYKCRDIAVKTANLG | 117 | GGUGUAUGUGCA | 319 | |
| 9802 | VSHLFMLDNTTPYLSDDDREKLTFLGCSGKKATKQNAPYV | | CCAUAUAUGUAG | | |
| | AASEKFKGQADMSMLSSVLQNVGKMYQDDKKKGMWSKSLR | | GUGACAUACAGC | | |
| | SYKANMPIPFKASCYRNLRFADYNDKEDKPHNGCFFTLMG | | | | |
| | IPFQCKFGKDRSGNRIIMQAVVDGKYKMCTSSLQIDGKKI | | | | |
| | FLLLCVDIPKKVVKLDESKTLYAFLGVMNPIVCTTDIKQK | | | | |
| | GDIDTDWKLWEIGTEAEFNYRRRQIQEAVKRCQVNNRYSR | | | | |
| | GGHGRFAKTKAIERWRAVERNYVDTKLHTYSKMLIDLAVK | | | | |
| | HKCGKIVLMNQLHREDAAKDDKFVLRNWSYHSLRTKIDYK | | | | |
| | AKMYGIKVEVEK | | | | |
| CasM.29 | MPVITRKIKLNLCTEGLSEDERKAQWKMLYRINDNLYRAA | 118 | GGGUACAUCCAA | 320 | RTRG |
| 0380 | NNISSKLYLDEHVSSMVRLKNAEYTSLVSDLMKAKKAEDE | | C | | |
| | AAITDLEAKIESLKSEMTAQEEAICCYATEMATRTLAGKF | | | | |
| | ASELDLDIYGQILAEVKSVVFKNFNSDSKEVREGNRSIRT | | | | |
| | YKKGMPIPFPWNKTIRLEAVKKELSGKHDEDEYDFYLNWY | | | | |
| | KSSRTDKKAIRFRLYFGKDKSNNQQIVKRCLHLDSTSSEN | | | | |
| | YQMQTSSIQMKKGPEGAELYLLLVVNIPQEQHALNKKIVV | | | | |
| | GVDLGINVPAYVATNCTEERKAIGDRDHFLNTRMAFSRRF | | | | |
| | HSFQRLKGTSGGKGRKKKLEPLERLREKERNWVHTQNHLI | | | | |
| | SRDVINFALQVKAATIQMEDLSGYGKDEEGNVKPENKELQ | | | | |
| | SKWSYFELQSMIKYKAAKCGIKVNLIAPAYTSQTCSWCGQ | | | | |
| | MGIRESTSFVCQNPECKQYGKDIHADYNAARNIARSNKIV | | | | |
| | KNE | | | | |
| CasM.29 | MRISKTLSLRIVRPFYTPEVEAGIKAEKDKREAQGQTRSL | 119 | GCUGUCAGUAGU | 321; | |
| 2901 | DAKFFNELKKKHSEIILSSEFYSLLSEVQRQLTSIYNHAM | | AGUAAAAAUGGG | 322 | |
| | SNLYHKIIVEGEKTSTSKALSNIGYDECKAIFPSYMALGL | | GGUACAUCCAAC | | |
| | RQKIQSNFRRRDLKNFRMAVPTAKSDKFPIPIYRQVDGSK | | AAAACAAGGAUU | | |
| | GGFKISENDGKDFIVELPLVDYVAEEVKTAKGRFTKINIS | | GAAAC | | |
| | KPPKIKNIPVILSTLRRRQSGQWFSDDGTNAEIRRVISGE | | | | |
| | YKVSWIEIVRRTRFGKHDDWFVNMVIKYDKPEEGLDSKVV | | | | |
| | GGIDVGVSSPLVCALNNSLDRYFVKSSDIIAFNKRAMARR | | | | |
| | RTLLRQNKYKRSGHGSKNKLEPITVLTEKNERFKKSIMQR | | | | |
| | WAKEVAEFFRGKGASVVRMEELSGLKEKDNFFSSYLRMYW | | | | |
| | NYGQLQQIIENKLKEYGIKVNYVSPKDTSKKCHSCTHINE | | | | |
| | FFTFEYRQKNNFPLFKCEKCGVECSADYNAAKNMAIA | | | | |
| CasM.29 | MEEKTKRLQKVAKFQIVKPVNMTWVELGKMLRDVRYRLWR | 120 | GAUGUGAACGAC | 323; | NTYN |
| 3203 | LANMAVCENYMRFYQWRIGKIDANENHKVKILNRRLREMI | | CUUUUUUUGCGG | 324 | |
| | IEEKQADAKELMRYSRDGVVSGYICGAFEKIHLSAIKNKS | | UGUACUUCGAGG; | | |
| | KWREVIRGKSNLPLFKRDLPIPINCSDHKPSLIAKTESDE | | GUGUACUUCGAG | | |
| | YEVDLRICQKPYPRVLLSTAKISGGERAILERLVSNKINS | | G | | |
| | LPGYRHRFFEIKEKPKGRWNLHVTYDFARSEATMLHSDII | | | | |
| | VGVDLGWSVPLYAAVNKGHARIGWRKLEPLAKRIRHLQKQ | | | | |
| | VKARRLSVQKGGQRDLAAPTARAGHGRKRILQPIEKLEGK | | | | |
| | IDDAYKTLNHQLSHCVIEFAKNNGAGVIQVENLEGLKDTL | | | | |
| | TGTFIGQNWRYNQLQNYIEYKAKEAGMELKKVNPCQTSQR | | | | |
| | CSNCGFIHRDFTFEYRQANKKNGKAAMFECPECSKKENYK | | | | |
| | PLNADYNAARNLATAGIEGKIRLQCEKQGIEYKGLPEE | | | | |
| CasM.29 | MSKITRKIEIIPDIEGLTHDESNKKCYGAFYTFDKNLYKV | 121 | GAUGUAAAUCAU | 325; | NNTG; TNTG; |
| 4190 | ANLLVSQLYGLDNLLSLMRLQNDEYVKCQSKLSLKSTTDA | | CUAUAAAAGAAA | 326 | WYTG; |
| | EKENLKKRMKKIDAELVSIKNGMAPKHPQTFAYRAVTNCV | | GGUACAUCCAAC; | | WNTG; TNTG |
| | YAKNIPSDILNTLKQDVYKHENDTKKEQFLGERSLITYKR | | GGUACAUCCAAC | | |
| | GMPVPFSIEKKHAIVCDGDNYYLPWFEDTRFRLNFGRDKS | | | | |
| | NNRAIIDNCIKTKRYKLCAAAKIQLKDKKLFLLVTVDIPA | | | | |
| | TETTSVKGKVMGVDLGVVNPAYVAVNDGPERSRIGNGEAF | | | | |
| | QKQRDVFRRRFRELQRSQLTQGGHGRKHKTKATETLRGKE | | | | |
| | RNWVQTENHRISREIVNLASRWKVECIQMESLKGYGKNQE | | | | |
| | GEVEDNHKRLLGRWSYFELQKDIEYKAAMVGIQVKYINPA | | | | |
| | YTSQTCHVCGQRGNRIERDTFICTNPECTCYNQAQDADMN | | | | |
| | AAINIAKSKDVVK | | | | |
| CasM.29 | MPTITRKIEMKLCTEGLSDQERKDQWNLLYHINDNLYRAA | 122 | GGGUACAUCCAA | 327; | NTCG; RTTR |
| 4406 | NNISSKLYLDDHVLSMVRLKHAEYLGLLRALEKAKKQKIP | | C; | 328 | |
| | DEEVIAELSQKVAAAEQEMDDQAKAICQYATEMSTQSLSY | | UGGUACAUCCAA | | |
| | RFATELETGIFTKILDCLKQGVFATFNSDTRDVKRGERSI | | C | | |
| | RTYKKGMPIPFAWNDSLRIELEDGEFYLRWYNGLRFRFDF | | | | |
| | GKDRSNNRLIVRRCLNMDEDYEGDYKLCNSSIQMVKREGL | | | | |
| | AKFFLLMVVNIPQEQVELNKKIVVGVDLGINAPAYVATNI | | | | |
| | TSERKQIGDREHFLNERMAFQRRFKSLQRLKGTTGGRGRA | | | | |
| | KKLEPLERLRKAEQNWVHTQNHLFSREVIDFAVKSRAATI | | | | |
| | HMEDLSGFGKDRDGNADDKKEFVLRNWSYYELQSMITYKA | | | | |
| | AKYGIKVEKIRPAYTSKTCSWCGHQGFREGITFICENPEC | | | | |
| | KKYGEKEHADYNAARNIANSIEIVKNNEE | | | | |
| CasM.29 | MKDYIRKTLSLRILRPYYGEEIEKEIAAAKKKSQAEGGDG | 123 | GUUGCAGAACCC | 329; | |
| 4601 | ALDNKFWDRLKAEHPEIISSREFYDLLDAIQRETTLYYNR | | GAAUAGACGAAU | 330 | |
| | AISKLYHSLIVEREQVSTAKALSAGPYHEFREKFNAYISL | | GAAGGAAUGCAA | | |
| | GLREKIQSNFRRKELARYQVALPTAKSDTFPIPIYKGFDK | | C; | | |
| | NGKGGFKVREIENGDFVIDLPLMAYHRVGGKAGREYIELD | | CGUACGUGGAUU | | |
| | RPPAVLNVPVILSTSRRRANKTWFRDEGTDAEIRRVMAGE | | GAAAC | | |
| | YKVSWVEILQRKRFGKPYGGWYVNFTIKYQPRDYGLDPKV | | | | |
| | KGGIDIGLSSPLVCAVINSLARLTIRDNDLVAFNRKAMAR | | | | |
| | RRTLLRQNRYKRSGHGSANKLKPIEALTEKNELYRKAIMR | | | | |
| | RWAREAADFFRQHRAATVNMEDLTGIKDREDYFSQMLRCY | | | | |
| | WNYSQLQTMLENKLKEYGIAVKYIEPKDTSKTCHSCGHVN | | | | |
| | EYFDFNYRSAHKFPMFKCEKCGVECGADYNAARNIAQA | | | | |
| CasM.29 | MPFKVLKLKIIKPVNMDWNELGQSIRDTRYRVYRLANLAV | 124 | GCUGCACUGCAC | 331 | NCCN; NCCR |
| 4655 | SEAYLAFHLWRAGKTDAIPKATAGQLNRRLRDMLLEEART | | CGCCCAUUGAUG | | |
| | KAVKDRKNTGEKGTEDDAKKAQKEMNKFSKTGALPDTVAG | | GUGUGCUCUAGG | | |
| | ALFMYKVKGLISKGKWTQVIRGKSALPTFRNNMAIPIRCD | | | | |
| | KKTQRRLERTENGVELELMIRNKPYPRVLLGTQGIGEGAE | | | | |
| | AIIERLLSNESQAEQGYKQRYFEVREDVNRTWWLYVCYAL | | | | |
| | PASTPPRLDPSKIVGVDLGFTCPMYAAISNGHARLGYRAF | | | | |
| | SSLAARVKALKLRTMRRRREIQRGGRTIVSGEAARSGHGR | | | | |
| | KRKLLGIEKLQGRVNQAYTTLNHQMSAAVVKFAIENGAGT | | | | |
| | IQVENLEGLREELSGTFLGQMWRYFQLQEFLQYKAEENGI | | | | |
| | VIRKVNPRYTSRRCSQCGHINKEFTRKARDRNAEGGYSAK | | | | |
| | FKCPDCEYEADADYNAAKNLAVDGIEGIIEKQCGSQGIVL | | | | |
| CasM.29 | MFLYKELKTMAKTNAEEGKIENKEKRLTKVAKFQIVKPVN | 125 | AUUGUAGGCGAC | 332; | NTYN; |
| 5201 | MTWPEFGKMLGDVRYRLSRVANMAVTEKYLESQQKRIGQK | | CUUUUUUUGCGA | 333 | WWTTN; |
| | IQRENTLVTIANRKLREMLKKEKVKEEELDRYSRDGAVSG | | UGUAGUUCGAGG; | | TTTYN |
| | YVTGPFEHNKLSAISKRFKEVLKGNMSLPNFKREMAIPIN | | AUGUAGUUCGAG | | |
| | CSNAKLSTIEKTETGEYVVDLRISQKPWPRVLLSTNRISN | | G | | |
| | GQREILERLAANKTFSDDGYKHLFFEVKQQGKDWFLSVTY | | | | |
| | SFPKSEAPKLHKDIIVGVDLGWSVPLYAAVNKGYARIGWQ | | | | |
| | KFRPLAERIKHLQKQVKARRITIQKGGQQDLATPTARTGH | | | | |
| | GRKRILRPIEKLERKIENAYTTLNHQLSHCVIEFAKNNGA | | | | |
| | GVIQIENLSGLANELSGTYIGQNWRYEQLQEYIRYKAEEA | | | | |
| | GIEVKHVNPCRTSQRCSECGFINDKFNFEYRQANRNNGMS | | | | |
| | AMFECPECKKNKKDYKPINADYNAAKNLTTANIDEIIRLQ | | | | |
| | CKKQGIEYKELPKD | | | | |
| CasM.29 | MSKITRKIELIPDIENLTHEESNQRCYKVFYNIDNKLYKV | 126 | AGUGUAUGAUUA | 334; | NNTG; TNTG; |
| 6640 | ANLLVCQLFGLDNLLSLMRLQNDEYVKCQSKLASKSISEE | | CCUGUAGUAUGA | 335 | WNTG |
| | TKRDIKKRMEAIDKELLARKDEIAPKHPQTFAYRAIKDSD | | GGUACAUCCAAC; | | |
| | YAKDLPSDIFNTLKQDVFKHFNETKKEQLRGERSLTTYKR | | AGGUACAUCCAA | | |
| | GIPVPFNLMKKNVIVSDGDNYYLTWFEETRFKLNFGKDRS | | C | | |
| | NNRAIIDNCLKINKYKLCTAAKIQLKNKKLFLLVTVDIPE | | | | |
| | TKNKIIKGKVMGVDLGVVHPAYVAVNDGPERSLIGDGDAF | | | | |
| | QKQRDVFRRRFRELQRCQLTQGGHGRKHKTKATENLRGKE | | | | |
| | RNWVQTENHRISREIVNLAIRWRVETIQMENLKGFGKDSD | | | | |
| | GDVETKHQRLLGRWSYFELQKDIEYKAAMAGIKVVYVNPA | | | | |
| | YTSQTCHVCGERGDRTERDTFICTNTECDCYGKPQDADMN | | | | |
| | AAINIARSKNIVK | | | | |
| CasM.29 | MTKVVKLPLICEQSDKDGNPIDYKKIYEILFELQRQTREI | 127 | GUUGCAAUUCGU | 336; | NTTC; TTNY |
| 6642 | KNKSIQYCWEFSNFSSDYYKQNHEYPKEKDILSYTLVGFV | | AUGCGCAGGUAA | 337 | |
| | NDKFKTGNDLYSGNCSTTVRGACGEFKNSKTDFLKGTKSI | | GUUUCGAG; | | |
| | INYKGNQPLDLHNKTIRFECIGKDYYAYLKLLNRPAFQRN | | GUUGCAAUUCGU | | |
| | NFSSSEIKFKVLVYDNSSKTIVERCIDNIYKISASKLIYN | | AUGCGCAGGUAA | | |
| | EKKKCWVLNLSYSFINNNVCELDENKILGVDLGIHYPICA | | GUUUCGAG | | |
| | SVNGERKFFKIDGGEIDHTRRKIEVRKKSLLKQGSSCGEG | | | | |
| | RIGHGIKTRNKPVYNIEDKIACFRDTANHKYSRALINYAV | | | | |
| | NNNCGIIQMEKLTGITADSDRFLKNWSYFDLQTKIEYKAK | | | | |
| | EAGITVVYIDPQYTSQRCSKCGYISKENRKVQAKFCCQKC | | | | |
| | GYEANADYNASQNIGIKDIDKIIKNTK | | | | |
| CasM.29 | VPITKTISLRILRPYYPPEIEAKIKAEKEKRKENGDTGSL | 128 | AAAACAAGGAUU | 338 | |
| 8142 | NSSYYRELKKEYPSIIINDEFFPLLSEMQRNITSIYNRTI | | GAAAC | | |
| | SHLYHRLIIKKESISTAKALSEGPYRDFKSTENSYIALGL | | | | |
| | RQKVQSNFRKKDLMAFKIALPTAKSDKFPIPIYMQTNFKI | | | | |
| | KESPDSDFIIELPLVEYIAKETKGKNKMFTKVEILSPPKV | | | | |
| | KNIPVILSTRRRKESGQWFSDEGTNAEIRRIISGEYKVSW | | | | |
| | IEIVKRTRFGKHDWFVNMVISFEESQEGLDPDVIGGIDIG | | | | |
| | VSKPLICAINNSLDRYIVKGDDIIAFNRRALSRRRSLLRR | | | | |
| | NRLKRSGHGSRNKLEPITVLTEKNERFKKSIMQRWAKEVA | | | | |
| | EFFKSKRASIVQMEELTGIKEREDFFSKTLRMYWNYGQLQ | | | | |
| | KTVENKLREYGIEVRYASPKDTSRRCHSCGHINDYFTFEF | | | | |
| | RQQNNFPLFKCMNCGIECSADYNAARNIAIAR | | | | |
| CasM.29 | MNRIYQGRVSKVEIPDGKDEWKKLDDGESALWQHHQLFQD | 129 | UGGUACAUCCAA | 339 | NTCG; RTTR |
| 8248 | DVNYLLAAFAALVPTSCEDDIWKDYQAAIERSWESYTGRQ | | C | | |
| | GIWDRPFENACVIVGCKKDASFKEFRRKLNSLTGSKASEK | | | | |
| | QKFEALKQLFEPATEAAKKLKKHDEPVEESLKGKAKDLFG | | | | |
| | STLVNLCAQKTKVTPRDVIAKQRNRASECTKKVNEGERLK | | | | |
| | WADVFYFKTDTSAAKWSREDAAKNIIQFLDKLLGEVEEKE | | | | |
| | KDAKTSDQKKKMADLAERLEKQKKPLAAWCNNSKTDLPTT | | | | |
| | EPTRKGSGGYDLKAAVLFSLQPDLDGFRDAFLLENQARLK | | | | |
| | EEFATTEKGDAAYIARMAGGVARPVFPFFCDVWAGKVNDE | | | | |
| | KIGQGIWPDFEKQAFSEVFTKIGQFIVRGRKFELRLAIAD | | | | |
| | QIIAKIETQKKSDARLQAVERIAEDLADELPDTAVDENGQ | | | | |
| | KRPYGIRERTLKGWRKVRPAWREALKKTPNLTAEDLIKQK | | | | |
| | NRMQERQREKYGSASLFDRLAKEPEIWNHDDKEDAVETWA | | | | |
| | DYVENLEEKAHLETERLFAPAHATLSPRFFRWSETNNKEH | | | | |
| | LEASSPDVPFELKADALDLSKKEKSQIKIHFWSPRLWRDG | | | | |
| | LRGKKENLDKDEPDQNWMPPVLRAFVKARKWPCDKQSFAG | | | | |
| | ASVRLAPRCKENIQLVFEPELHTEILSAKWKENFPFSPAK | | | | |
| | NKESESVGLFWPRTKEDKVLWFDKGETRCLGVDLGLINSA | | | | |
| | AWQILQATNKDATAKAPRLRHRLNPDSEKAAWFAHSITNG | | | | |
| | IVRVAGEDCWGWRKFAPDEKAKLRAELKKPAGKRNALCRK | | | | |
| | FLSLNREIEFETATHSFLPELSGSGGRNPTDDETKEAAEF | | | | |
| | FSTLKTKGFDITDRQPSWGKNLSFPKQNDELLWGLKRVRA | | | | |
| | QLFRLNRWSEQLGKERDSKPYQSAIEIIGNLRSDDPLIEL | | | | |
| | ATLKSEPKRLKSRIAELAGEYLDCFKTLLPRIADRILPWR | | | | |
| | RGHWSWKPCDNDWHRMELDASKPRPEALLAGQRGISLPRL | | | | |
| | NQLKDLRQLAQSLNHLCRRKQIKRNETVPEPFEDCRQAME | | | | |
| | DAREDRAKKIAHEVFAIALGVELAPPPPDKQERKQTESLH | | | | |
| | GVYRCLERGPVNFIALENLGGYNPSAKQGRRENRQLSSWL | | | | |
| | KGRIHKILGELCEMVGMPIVLVNAEYTSRFSAKDHSPGFR | | | | |
| | AEEVQTDDSRRSFWQRKAKEEPSGWQNEFLCWLNKVPDGK | | | | |
| | SLLLPKKGGEFFVPLGEGTSLYHADLNAAYRIALRALAHR | | | | |
| | DRAELLGQTWIEKKPYLVDVAGVFPDSILRNGCAFKTISS | | | | |
| | SERLWEKVNGDLAMQRCREINLARFASWKIALPQQIISEA | | | | |
| | LPPDEEDDIPM | | | | |
| CasM.29 | MSEATKTLAYRYRLRLTPAQEDILDRSQEQLRLVWNHLVR | 130 | UGGUACAUCCAA | 340 | RTTR |
| 8264 | SQHKVEHEWRHGRAASIKNELLELSLAKNATGQAIPSARK | | C | | |
| | ITEERGVSMEEALRLMRQKFVEKVSAIPLRKKDGSRCLRI | | | | |
| | ARRKMATEYAVTVVNAKFKHYYGLGARMCKVLRDKFQKCS | | | | |
| | DMWIKGKFRRPRFKRKGESVALQRQVQSNSPFKLKRFSDL | | | | |
| | SALGGQALKKCEVIIHRPLPDSAEIKQIAVSGRRGQRHLI | | | | |
| | VMFKAASSDVAKNFPATNRTAGVDPGIKVALTITPLDSPD | | | | |
| | FGTSDKIEKQPDLARDACFLKRLRRLQRKHDRQRRQNNPE | | | | |
| | CFDEKGRWIKGKRLHNESKNMQRTQSRITAMNTHLAESRR | | | | |
| | DFYHNAACEILRSFDNVAVGKWRPAQTRQRKPTTPSPKGL | | | | |
| | GAARRATNRISYDHAISLFISYLKDKAERSVTTKHVQEVS | | | | |
| | EFGSTRSCPKCGKLTGPVGTEGLAVRDWTCVNCNTTFQRD | | | | |
| | AASAWQIAKRFKAEVASTSQPAESQDSANSASVLTQV | | | | |
| CasM.29 | MPTLTRKVELYVVGDKEEVSRVYDYIRLAMNATYKCFNEC | 131 | UGGUACAUCCAA | 341 | NTCG |
| 8446 | MTALYIAQVKEDTKEDRKELNHLYSRQTYTKKETAFINDI | | C | | |
| | VFPEGLALAAYVNRMAQQKFVTSLKNGLMYGCVSLPTFKK | | | | |
| | DCAVPLHVKFVSLAGEKGTNTGFYHEYADVNDLVNALEYD | | | | |
| | NSPKVFLRFPNNITFGVVFGNPYRGREQRSVFSKIFLGEY | | | | |
| | KIQGSSIQINSRGKIILNLSMEVPKKKMEHIEGRVVGVDV | | | | |
| | GLAIPAMCAINDDDYTRSAIGNIDDFLKVRTQIQSQRRRL | | | | |
| | QKSLKNTSSGHGRTKKLKPLERIAEKERNFANTYNHMVSK | | | | |
| | RVVDFAVKNGASQINIEDLSGFAKDKNGKSVEDDNMKRVL | | | | |
| | SNWSYFELQQQIRYKAEQYDIKVRTVNPAYTSQTCSYCGQ | | | | |
| | IGKRETQSKFVCTNPDCKCHKMYKKDWFNADFNAARNIAL | | | | |
| | STDYTDDEDGKKTKKKKSAKKKPEKKTEEA | | | | |
| CasM.29 | MSGASGQITRDNKAQRSGPNKGEMSEDHSSTKRPKRVVKV | 132 | GCUGCAAGAGCU | 342 | NNCC |
| 8612 | AKYRIIKPVGEMTWPELGEILRTVRYRVFRLGNLAVSEAY | | CCUAAUUUGAGG | | |
| | LNFHAFRTGKAEEFKSETIGKLSRRLRDMLISEGVKKEDI | | GGUGCAUACAGG | | |
| | DRYSATGAVPDTVAGALGQYKVRGITSPAKWRQVIRGTVS | | | | |
| | LPTFRNDMAIPVRCDKPAQRRLEKAKSEEVEVDLMICRKP | | | | |
| | YPRVLIGTADLGGGQQAILERLLDNKDNSSDGYRQRLFEI | | | | |
| | KQDTQSKKWFLFVTYDFPSSGALPLDPNVAVGVDLGVSVP | | | | |
| | LYAAINNGHARLGRRQFQALGSRIRSLQTQVDARRRAIQR | | | | |
| | GGRSDVSQSTARSGHGVRRKLQPTEKLRKRIDRSYSTLNH | | | | |
| | QLSAAVVEFAKNQGAGTVQMEDLGGLREELTGTFIGARWR | | | | |
| | YHQLQQFLEYKCDEAGITLNKVNPMYTSRRCSECGFIDKD | | | | |
| | FDRAFRDRSRSDGRVARFICPECSYEADPDYNAARNIATL | | | | |
| | DIDKLIRVQCQKQGLKYDAL | | | | |
| CasM.29 | MIITRKIELWLSEDDNELRKAKWSYLKELNDEVYRAANFI | 133 | GAUAGUUUUAAC | 343; | NTTR; WTTR; |
| 9584 | VNNQYFNEILENRVIMQDTRLIDIDSEIRKLYKSREKNKE | | UUCCAUUUGAAA | 344 | RTRG |
| | KIDELKKIKKIRYQEAKNFYQTSKQNVTYQLTSREFPNIP | | UGUAAAUGCAAC; | | |
| | ANIVTSLNASIIKTLKTEWNEIKSGKRAVRNYRKGMPIPF | | AUGUAAAUGCAA | | |
| | NFSSSQKWFENKGEDIFLNWLGGLKFKLFFGRDKSNNRAI | | C | | |
| | VERAINKEYKYADSSIQLKDKKIFLLFVVDIPYEKANLNK | | | | |
| | NIAAGVDLGIAFPAFCALSEGYSRLSIGNKEDLLKVRLQM | | | | |
| | QSRRKRLQKALKITSGGKGRTKKLKALESLINKEKNYVTT | | | | |
| | YNHKVSYQVIKFAKDNKAGIIKLEFLEGFGEDEKNKFILR | | | | |
| | NWSFYQLQKMIEYKAKREGIEVLYIDPYHTSQTCAICGNY | | | | |
| | EEGQREKQEDFICKNPECKNFEKIVNADYNAALNIAKSNK | | | | |
| | IVSSSEQCEYNKKHENNVL | | | | |
| Cas14a.1 | MAKNTITKTLKLRIVRPYNSAEVEKIVADEKNNREKIALE | 134 | GAATGAAGGAAT | 345 | TTTR |
| | KNKDKVKEACSKHLKVAAYCTTQVERNACLFCKARKLDDK | | GCAAC | | |
| | FYQKLRGQFPDAVFWQEISEIFRQLQKQAAEIYNQSLIEL | | | | |
| | YYEIFIKGKGIANASSVEHYLSDVCYTRAAELFKNAAIAS | | | | |
| | GLRSKIKSNFRLKELKNMKSGLPTTKSDNFPIPLVKQKGG | | | | |
| | QYTGFEISNHNSDFIIKIPFGRWQVKKEIDKYRPWEKFDF | | | | |
| | EQVQKSPKPISLLLSTQRRKRNKGWSKDEGTEAEIKKVMN | | | | |
| | GDYQTSYIEVKRGSKIGEKSAWMLNLSIDVPKIDKGVDPS | | | | |
| | IIGGIDVGVKSPLVCAINNAFSRYSISDNDLFHFNKKMFA | | | | |
| | RRRILLKKNRHKRAGHGAKNKLKPITILTEKSERFRKKLI | | | | |
| | ERWACEIADFFIKNKVGTVQMENLESMKRKEDSYFNIRLR | | | | |
| | GFWPYAEMQNKIEFKLKQYGIEIRKVAPNNTSKTCSKCGH | | | | |
| | LNNYFNFEYRKKNKFPHFKCEKCNFKENADYNAALNISNP | | | | |
| | KLKSTKEEP | | | | |
| CasM.26 | MSVLTRKVQLIPVGDKEERDRVYKYLRDGIEAQNRAMNLY | 346 | AAGGAUGCCAAA | 347 | NNTN; TNTR; |
| 5466 | MSGLYFAAINEASKEDRKELNQLYSRIATSSKGSAYTTDI | | C | | TNTG |
| | EFPTGLASTSTLSMAVRQDFTKSLKDGLMYGRVSLPTYRK | | | | |
| | DNPLFVDVRFVALRGTKQKYNGLYHEYKSHTEFLDNLYSS | | | | |
| | DLKVYIKFANDITFQVIFGNPRKSSALRSEFQNIFEEYYK | | | | |
| | VCQSSIQFSGTKIILNMAMDIPDKEIELDEDVCVGVDLGI | | | | |
| | AIPAVCALNKNRYSRVSIGSKEDFLRVRTKIRNQRKRLQT | | | | |
| | NLKSSNGGHGRKKKMKPMDRFRDYEANWVQNYNHYVSRQV | | | | |
| | VDFAVKNKAKYINLENLEGIRDDVKNEWLLSNWSYYQLQQ | | | | |
| | YITYKAKTYGIEVRKINPYHTSQRCSCCGYEDAGNRPKKE | | | | |
| | KGQAYFKCLKCGEEMNADFNAARNIAMSTEFQSGKKTKKQ | | | | |
| | KKEQHENK | | | | |

TABLE 1.1 provides exemplary amino acid alterations relative to SEQ ID NO: 346 useful in compositions, systems, and methods described herein.

TABLE 1.1
Exemplary Amino Acid Alterations Relative to SEQ ID NO: 346

| Effects | Amino Acid Alterations |
|---|---|
| | At least one substitution (i.e., with R, K or H) selected from K58, I80, T84, K105, N193, C202, S209, |
| | G210, A218, D220, E225, C246, N286, M295, M298, A306, Y315, and Q360 |
| Enhanced nuclease activity relative to | I80R, T84R, K105R, C202R, G210R, A218R, D220R, E225R, C246R, Q360R, I80K, T84K, G210K, |
| the wild-type effector protein | N193K, C202K, A218K, D220K, E225K, C246K, N286K, A306K, Q360K, I80H, T84H, K105H, |
| | G210H, C202H, A218H, D220H, E225H, C246H, Q360H, K58W, S209F, M295W, M298L, Y315M |
| | Double mutations: D220R/A306K, D220R/K250N |
| Reduced or abolished nuclease | D237A, D418A, D418N, E335A, E335Q |
| activity relative to the wild-type | |
| effector protein | |

TABLE 1.2 provides exemplary amino acid alterations relative to SEQ ID NO: 12 useful in compositions, systems, and methods described herein.

TABLE 1.2
Exemplary Amino Acid Alterations Relative to SEQ ID NO: 12

| Effects | Amino Acid Alterations |
|---|---|
| | At least one substitution (i.e., with R, K or H) selected from I2, T5, K15, R18, H20, S21, L26, N30, E33, |
| | E34, A35, K37, K38, R41, N43, Q54, Q79R, K92E, K99R, S108, E109, H110, G111, D113, T114, P116, |
| | K118, E119, A121, N132, K135, Q138, V139, N148, L149, E157, E164, E166, E170, Y180, L182, |
| | Q183, K184, S186, K189, S196, S198, K200, I203, S205, K206, Y207, H208, N209, Y220, S223, E258, |
| | K281, K348, N355, S362, N406, K435, I471, 1489, Y490, F491, D495, K496, K498, K500, D501, V502, |
| | K504, S505, D506, V521, N568, S579, Q612, S638, F701, and P707 |
| Enhanced nuclease activity relative to | T5R, L26R, L26K, A121Q, N148R, V139R, S198R, H208R, S223P, E258K, N355R, I471T, S579R, |
| the wild-type effector protein | F701R, P707R, K189P, S638K, Q54R, Q79R, Y220S, N406K, E119S, K92E, K435Q, N568D, and |
| | V521T |
| | Double mutations: L26K/A121Q, L26X/A121Q, K99R/L149R, K99R/N148R, L149R/H208R, |
| | S362R/L26X L26X/N148R, L26X/H208R, N30R/N148R, L26X/K99R, L26X/P707R, L26X/L149R, |
| | L26X/N30R, L26X/N355R, L26X/K281R, L26X/S108R, L26X/K348R, T5R/V139R, I2R/V139R, |
| | K99R/S186R, L26X/A673G, L26X/Q674R, S579R/L26K, F701R/E258K, T5R/L26K, L26X/K435Q, |
| | L26X/G685R, L26X/Q674K, L26X/P699R, L26X/T70E, L26X/Q232R, L26X/T252R, L26X/P679R, |
| | L26X/E83K, L26X/E73P, L26X/K248E, L26X, T5R/S223P, S579R/S223P, L26X/S223P, T5R/ |
| | A121Q, L26X/A696R, S198R/I471T, L26X/N153R, L26X/E682R, L26X/D703R, Q612R/L26K, |
| | L26X/I471T, K348R/L26K, S579R/I471T, L26X/V228R, T5R/S638K, S579R/K189P, S579R/E258K, |
| | L26X/K260R, L26X/S638K, S579R/Y220S, T5R/I471T, L26X/F233R, L26X/V521T, F701R/A121Q, |
| | L26X/G361R, S198R/E258K, L26X/S472R, T5R/Y220S, L26X/A150K, L26X/S684R, L26X/E157R, |
| | L26X/K248R, F701R/L26K, S198R/N406K, S198R/Y220S, S198R/S638K, S198R/V521T, |
| | S579R/A121Q, K348R/Y220S, S198R/K189P, L26X/E242R, L26X/K678R, T5R/N406K, |
| | L26X/I158K, T5R/V521T, L26X/N259R, L26X/K257R, L26X/K256R, T5R/K189P, L26X/C405R, |
| | S579R/V521T, S579R/N406K, T5R/K92E, T5R/E258K, L26X/I97R, S579R/S638K, T5R/K435Q, |
| | F701R/S638K, L26X/L236R, F701R/I471T, Q612R/S223P, F701R/S223P, S198R/E119S. |
| | S579R/K92E, L26X/E715R, Q612R/I471T, F701R/Y220S, S198R/S223P, and L26X/K266R, wherein |
| | X is selected from R and K. |
| Nickase activity | E157A, E164A, E164L, E166A, E166I, E170A, I489A, I489S, Y490S, Y490A, F491A, F491S, |
| | F491G, D495G, D495R, D495K, K496A, K496S, K498A, K498S, K500A, K500S, D501R, D501G, |
| | D501K, V502A, V502S, K504A, K504S, S505R, D506A; |
| | deletion of S478-S505 of SEQ ID NO: 2; |
| | deletion of S478-S505 of SEQ ID NO: 2 and insertion of the sequence of |
| | SDLYIERGGDPRDVHQQVETKPKGKRKSEIRILKIR (SEQ ID NO: 7); |
| | deletion of S478-S505 of SEQ ID NO: 2 and insertion of the sequence of |
| | SDYIVDHGGDPEKVFFETKSKKDKTKRYKRR (SEQ ID NO: 8); |
| | an amino acid sequence that is at least 90%, at least 95%, at least 97%, at least 98%, at least 99% |
| | identical, or is 100% identical to SEQ ID NO: 9; |
| | an amino acid sequence that is at least 90%, at least 95%, at least 97%, at least 98%, at least 99% |
| | identical, or is 100% identical to SEQ ID NO: 10 |
| Reduced or abolished nuclease | D369A, D369N, D658A, D658N, E567A, E567Q |
| activity relative to the wild-type | |
| effector protein | |

TABLE 2 provides exemplary effector protein CasPhi. 12 engineered variant sequences.

TABLE 2
Exemplary Effector Protein CasPhi.12 Engineered Variant Amino Acid Sequences

| Effector | | SEQ |
|---|---|---|
| Protein | | ID |
| Variant | Amino Acid Sequence | NO: |
| j12_L17_ | MIKPTVSQFLTPGFKLIRNHSRTAGLKLKNEGEEACKKFVRENEIPKDECPNFQGGPAIANIIAKSREFTEWEIYQSSLAIQEVIFTL | 578 |
| 18_del1 | PKDKLPEPILKEEWRAQWLSEHGLDTVPYKEAAGLNLIIKNAVNTYKGVQVKVDNKNKNNLAKINRKNEIAKLNGEQEISFEEIKAFD | |
| | DKGYLLQKPSPNKSIYCYQSVSPKPFITSKYHNVNLPEEYIGYYRKSNEPIVSPYQFDRLRIPIGEPGYVPKWQYTELSKKENKRRKL | |
| | SKRIKNVSPILGIICIKKDWCVFDMRGLLRTNHWKKYHKPTDSINDLFDYFTGDPVIDTKANVVRFRYKMENGIVNYKPVREKKGKEL | |
| | LENICDQNGSCKLATVDVGQNNPVAIGLFELKKVNGELTKTLISRHPTPIDFCNKITAYRERYDKLESSIKLDAIKQLTSEQKIEVDN | |
| | YNNNFTPQNTKQIVCSKLNINPNDLPWDKMISGTHFISEKAQGSSGDYKWFQDYKPKLSKEVRDALSDIEWRLRRESLEFNKLSKSRE | |
| | QDARQLANWISSMCDVIGIENLVKKNNFFGGSGKREPGWDNFYKPKKENRWWINAIHKALTELSQNKGKRVILLPAMRTSITCPKCKY | |
| | CDSKNRNGEKFNCLKCGIELNADIDVATENLATVAITAQSMPKPTCERSGDAKKPVRARKAKAPEFHDKLAPSYTVVLREAV | |
| j12_L17_ | MIKPTVSQFLTPGFKLIRNHSRTAGLKLKNEGEEACKKFVRENEIPKDECPNFQGGPAIANIIAKSREFTEWEIYQSSLAIQEVIFTL | 579 |
| 18_del2 | PKDKLPEPILKEEWRAQWLSEHGLDTVPYKEAAGLNLIIKNAVNTYKGVQVKVDNKNKNNLAKINRKNEIAKLNGEQEISFEEIKAFD | |
| | DKGYLLQKPSPNKSIYCYQSVSPKPFITSKYHNVNLPEEYIGYYRKSNEPIVSPYQFDRLRIPIGEPGYVPKWQYTELSKKENKRRKL | |
| | SKRIKNVSPILGIICIKKDWCVEDMRGLLRTNHWKKYHKPTDSINDLFDYFTGDPVIDTKANVVRFRYKMENGIVNYKPVREKKGKEL | |
| | LENICDQNGSCKLATVDVGQNNPVAIGLFELKKVNGELTKTLISRHPTPIDFCNKITAYRERYDKLESSIKLDAIKQLTSEQKIEVDN | |
| | YNNNFTPQNTKQIVCSKLNINPNDLPWDKMISGTHFISEKAQVSNKSEGSSGDYKWFQDYKPKLSKEVRDALSDIEWRLRRESLEFNK | |
| | LSKSREQDARQLANWISSMCDVIGIENLVKKNNFFGGSGKREPGWDNFYKPKKENRWWINAIHKALTELSQNKGKRVILLPAMRTSIT | |
| | CPKCKYCDSKNRNGEKFNCLKCGIELNADIDVATENLATVAITAQSMPKPTCERSGDAKKPVRARKAKAPEFHDKLAPSYTVVLREAV | |

TABLE 3
Exemplary RDDP Sequences

| RDDP | | SEQ |
|---|---|---|
| Ref | Amino Acid Sequence | ID: |
| 2691297 | VNHYLRLTSLPALYAGWERVAANDGGPGVDRVSINKEDVALDRDLERLHRELVSFTYRCMPLRRYEIPKLNGKIRTLAVPTVRD | 485 |
| | RVVQSSALIVLQPVIERELERCSFAYRPGLSRLTAIEQIRQLREQGYRWVVDADIESFFDNVEFGLLLRRFRELVPENHTVALI | |
| | EQWLKAPVLFHGRLIKRIKGLPQGSPISPVLANLFLDKFDEALLAHNHKLIRYADDFIILCKTKPRAEEALRLTEELLAGLHLR | |
| | LNAEKTAVTSFDQGFKYLGAIFVKSIIIPQSGTKPQRRKKQMNAQSPDPPLRQNEKRRKSAAAKKKEEITVLAQALLEALNAKQ | |
| | MTTADLVRTPPTRPPVPNGAPEPPSAPPNVSPFMRTLYIQEQGCWLRLDGERFVVATGGEEGVALYEIPTVKVEQIMIFGTCLI | |
| | TPAAMRYALLHTIPITLLSSRGQYFGRIESTYGADITLERTQFLHSVDTAFVLQTARTIVQAKLRNTKALLQRHQTKHEMISAA | |
| | IVQLTRLEDQVANATSVDQVRGYEGSGSAVFFDVEDELMPGKGFTFEKRTRRPPTDPVNAMLSFGYTLLENNIYSMARLHRLNP | |
| | YVGSLHAALSEHKQALTCSY | |
| 2691299 | MYRCVKLPVAENMPNTDSQLNTVGWDEIDWRKVERYVYKLHKRIYAASRRGDVKQVRKLQKTLMSSWSNQVLSVRKVTQDNRGK | 486 |
| | RTAGVDGIHILSSEARINLAGSLKITGKSHPTRRVWIPKPGTEEKRPLGIPTIYDRVVQNLVKTALEPEWEAIFEPNSFGFRPG | |
| | RSCHDAIWQIKNSIQNRPKFILDADIAKCEDRINHLALLQKLGYTGKIRQQIKAWLKSGVIDRGAFTATSEGTMQGGVISPLLA | |
| | NIALHGMEKMLMEFAKTLDMRRNDKPNCQISWQQKVKSLTFVRYADDEVLIHKDLNVVRRCRDLISEWLKDIGLELKPSKTRIA | |
| | HTKKIRERGSPEAKRYLYRGGNATRA | |
| 2691301 | MMAHRKIKANHGSKTKGMDKQNVIDFKNMVTEEYLSEIKLELLDYKSGLIRRKEIPKPNGGVRPLGISCYKDKIIEEAIKQVIE | 487 |
| | PIVEAKFHPDSYGFRPGRSAQEAIQMSQTLVRAGYTYACEFDIKKCFDNIKHRLIRKKLWALGIRDTRLLTIISKRLRANIYDP | |
| | KTKTVSQSTLGTVQGGVLSPLLANVVLNSLDWWIASQWERFDFGIEENDYKNYASFHAAMNYRLNKTRLKSGRIVRYADDFVIF | |
| | CKTEMEAKAWYFAVKDWMKCNLKLEISEEKSRIVDLTKDSIDFLGFELKANKEGKKCVKSVDRSNRGSYKYTKTSVTTKNLNKI | |
| | IQTLKTKIKIILKTSDKKAKAVLITLLNITILGYHNYWRIASESSINFYNIYQRTKLSWWKLRRQADSKTHISPAFRKLYKEYV | |
| | DSAFSIDGNTIYPIWGCRHTTLNPKKRDYNWYEDSPSIPENISSQMKIMCCNPIIHRSVEYNDNRLSKYSMQLGKDYITGKFVM | |
| | AQDVHCHHKTMVSKGGKDNFDNLVILSKVTHGWVHQVNPEIPYMTKVQMDRLNDLREKCGREPLKNKKKSLKSEN | |
| 2691303 | MRKWYSLIDKVYARANLLEAFDKVRRNKGGKTSGIDGISVQAFKEHLTANLCQLHEELRSGTYRPQAVKRVYIDKEDGGKRPLG | 488 |
| | IPTVKDRVVQQALLNVLQPIFEPEFHPSSYGYRPGRSAHHAIAKAERFTRYYGLDHVVVMDLSKCFDTLDHELILSSVNEKVSD | |
| | GKVLRLIDSFLKSGIIDKGKEEPTEKGSPQGGIISPLLTNIYLNKFDQYMKTLGIRIVRYADDILVFASTKSEAGKYRAIATTY | |
| | LEGELMLTVNREKTHLTTPYKGIPYLGFEIHNYGVSASEKSIRKFKEKVRKLTPRNQGYKLDYYLTELNFLLKGYSMYFRVARS | |
| | KSLFRNLMSWVRRRLRMMIMKSWKSWKGLHKQLRRMGYKGSLEKISVTRWRNSSCQLLHMALPNTWFDEQHLENMEKVTTGTLH | |
| | QYYNFVLNKV | |
| 2691305 | VNRAVLTLADLARPDALFDAWQQAERRKAGPGTDGQTVQVFGQQVEEHLEGLSKEIREGGYQPRPAQRIWMVKDSGGERGITIF | 489 |
| | TVRDRVALGAMRRTLGRLIGPTLSTASFAYREGLGALRAAHRLIEHRRSGRGWVVRSDIKEFEDTISHEILLEQLRVFLDARAL | |
| | ELFGTILRTPIRDGYGISVPERGVAQGSPISPMLANLYLSGFDAALNCDGRGFVRFADDEVIAALSVKGAAEGLEIAQAQLEGL | |
| | ELRLNDDKTRIVSFEQGFDFLGFHFDADTVRVSETSLAEFKAHLEALLVSRFEQFSPGAVKKANDLIRGWRNYFQLGDVRKDYA | |
| | ALEEWIEARFGHKARQLERLLPDARGQRVPQLGGYGRVSRERQPRRSGQANTASARPQKRQRPYPNVKLDANDTPLTVRRNKAV | |
| | LNVATLPALPETRVSRAILDELEQASLFHRANTACRWSTPDAADAVSNLAAAMSGRIPIRQAIEAYDKALFAQLKPGADPRGAR | |
| | GTLRANLRRIAWAALLEAGIDLGPEQPRKTPTLGNALADAYECLTADLALLAASRNDQLHAPQGAFERSLAFKPAYQRPGVQGQ | |
| | AHATAWMMILRLEAEAMRRMLETNGQTVYRVWRFGLP | |
| 2691307 | MEAVVERENMIAAYKRVVANRGAAGVDGMTVDALKAYLQPHWPRIRKELLEGRYRPQPVLEVEIPKPAGGMRKLGIPTVVDRLI | 490 |
| | QQAIHQVLEPIFDKDFSESSYGFRRGKSAHQAVKQAREYVKEGRRWVVDIDLEKFFDRVNHDILMARVARKVRDKRILRTIRQY | |
| | LQAGIMTGGLVTARTEGTPQGSPLSPLLSNIMLDDWDKELERRGHRFCRYADDCNVYVHSKRAGERVMESLRQFLEKRLRLRVN | |
| | TEKSRVDHPWQLKFLGYSMTWEKNTRLKVASQSVERFKSKLRLIFRQGRGRNVQIVINELNLVLRGWVQYFRLAEVKNIFEEMD | |
| | GWIRRKLRCTLWKQWKRTYTRAKNLMKRGLEEVRAWQSATNGRGAWWNSAASHMNEAYPKSYFDKAGLVSLLQLLWQIQLT | |
| 2691309 | MSEPMEDKPEAVSERIKRAGELLRHWMWTEPTVWTERMLTALAQGVKGGKWFSLIDKIHPERTLRAAYGHVAANKGSAGVDHVT | 491 |
| | TTMFGDHLDEEVSKLSEQLRNGSYRPQAIRRHYIPKPGSQEKRPLGIPTVRDRVVQTALRMVIEPIFEREFAEDSYGFRPGRGC | |
| | KDALRRVDELLKDGYIYIIDADLKSYFDTIPHDRLMDLVRQKISDGRVLNLIEAFLKQGILENLQEWIPESGSPQGAVVSPLLS | |
| | NIYLDPLDHKMAQEGYQMVRYADDEVILCRTPEKAERALAAVQEWTAAAGLTLHPTKTRIVNATIDSFEFLGYRFSKGKRFPRT | |
| | KSMQKLKDTIRAKTKRINGQCLPAIIGSLNPTLRGWFEYFKHSYKTTFPELDGWIRGRLRSILRKRQKKRGRGRGSDHQRWPNA | |
| | YFADLGLYSLKTAHKLACQSSSR | |
| 2691311 | MKGRMQKKSIDTCQQKNRTESDGYVEGQTFIWITENNLTDKYQLGNGLLEFILSPNNLNQAYHQVKRNKGAGGIDKMEVESLKD | 492 |
| | YLVRHKDELIKSILEGKYRPSPVRRVEIPKETGKTRQLGIPTVVDRVIQQSISQVLSQIYEKQFSDTSYGFRPRRGAHQALRTC | |
| | QKNITEGYVWSVDMDLEKFFDTVNHSKLIEVLSRTIKDGRVISLIHKYLNAGVIVRNNFEETEVGVPQGGPLSPLLSNIMLNEL | |
| | DREIENRGHRFVRYADDMVILCKSKRSAVRTLENLLPFIEGKLFLKVNKEKTSVAQISKIKFLGYSFYRLNGECQLRVHPKSVL | |
| | KMREKMKSLTSRSNGWGNERRKESLRQYIVGWVNYFKLANMKKLLLRTDEWYRRRLRMVIWKQWKRISTKLINLIKLGINKYKA | |
| | WELVNTRKGY | |
| 2691313 | MSLATPERIRSLQRKLYCKAKAEPAFRFYTLYDKICRADILAHAYEVARSNAGAPGVDGRTFEHIEALGLQEWLAGLREDLVAR | 493 |
| | TYRPMPVRRVMIPKPGGGERPLGIPTIRDRVIQTAAKLVLEPIFEADFEDSAYGYRPRRGASDAIKEVHRLLCRGHTDVADADL | |
| | SKYFDTIPHAQLLRCVARRIVDGEVLRLIKLWLKTPIEGRDADGKRRLTGGKRSRCGTPQGGVVSPLLANLYMNRFLKHWRFTG | |
| | CGEVFRAQIIAYADDFVILSCGHAAEAMMWTKAVMTKLGLNLNEAKTSIRDARKERFEFLGYSFGPHYWWKNGQRYLGASPSKK | |
| | SVQRIKAKISELLVPGNKGSWPDVRDQLNRLLSGWSAYFNHGTRVPAYRAVDAHVYDRVQNFLCRRHNVRRRGTSRFSFGDVFG | |
| | ELAVLHLM | |
| 2691315 | MDEDELTEAFYQLRKDAAAGIDEMTATEYEVNLGGRIADLHRRLVAQEYRAQPARRVWIPKGDGGQRPLAILVLEDKIVQRAVA | 494 |
| | MLLEAIYEPHFCGFSYGFRREHNAHQALTYLRQQCLELGINWIIDADIQKFFDTISWEQLRAILQRRMNDGAVLRLIGMWLHVG | |
| | VLEAGQVINQELGTPQGAPISPILANIFLHIVLDEWFQSQVRPRMRGNCFLVRYADDFVMGYSLKGDAERVYEVLPKRFERYGL | |
| | RIHPEKSRMVQFSRPYWQRGKGPGSFAFLGFTHYWAKTRSGSWTIKRKTQGKRLSRFLSGIADWCKENLHEAIGEQHRILSAKL | |
| | RGHYQYYGVRGNFKLLEVAYEHTRGMWKKWLGRRNSMNRMSWEKFVEQVESVFTLPLPRIVHAF | |
| 2691317 | MPKTFYSLYDRLLHRRALARAFAKVRRAKGAKTPGQDGQTAADFAADEEAELDRLVHELRTKTYRPQPVRRVTIPKPGGGERNL | 495 |
| | GIPAVRDRVVQQSLLDILQPIFDPDFHPSSYGYRPGRSSQQAVAKATRFVRQYGLAHAVDMDLSKCFDRLDHDLIVASFRRRVR | |
| | DGSILALMRMFLESGVMIGDDWEATEVGSPQGGVISPLISNVYLDAFDQEMKRRGYRIVRYADDILILCRSKSAALHAREVATA | |
| | ILEGPLRLTVNKEKTHLVHASQGVKFLGVEIGLTWTRIQDKKVAAFKAQVKRLTRRNSPVNLGQVIADLNPVLRGFANYFRMAN | |
| | CKGLLSELMSWIRRRLRAKQLALWKKPGRLHRRLRQLGYQGDFPKIRMTRWRNSRSLQASWSLPNGWFRELGLYELTAVETGVL | |
| | PQAT | |
| 2691319 | MSELLEKILSKDNMNTAYKRVCANKGAGGVDDVTVEELGAYVKENWESIREQIRRREYTPQPVRRVEIPKPDGGERKLGIPTVM | 496 |
| | DRVIQQGIVQIISPMCEPLFSKWSYGFRPNRSCEMAIRQLLEYLNEGYEWIVDIDLEKFFDNVPQDRLMSLVHDIIKDGDTESL | |
| | IRKYLKAGAMTPQGYKETKLGTPQGGNLSPLLSNIMLNELDKELEARGLRFTRYADDCVIAVKSESSAKRVMRTVTDWIQRKLG | |
| | LKVNMTKSQITRPQKLKYLGFGFHKDSKAKEWKCRPHQESVKKFKVRLKELTCRKKPGTVTSKIAKINQVTRGWINYYALGSMK | |
| | TAMTEIDAHLRTRLRVIIWKQWKVPKKRQWGLQKLGVSKDLARLTAYCGDSYYWVATKTCVVRAISKEVFAKSGLVSCLDYYNE | |
| | RHALKLC | |
| 2691321 | MEQVVERSNMLAALKRVERNGGAPGIDGVPTERLRDQIRAEWRRIREELLAGTYRPEPVRRVEIPKPGGGERLLGIPTVMDRLI | 497 |
| | QQALLQVLTPIFDPQFSDSSYGFRPGRRAHDAVKRARQYVEEGYEWAVDLDIEKFFDRVNHDILMARVARRVTDKRVLTLIRRY | |
| | LQAGVMVNGVVMETAEGTPQGGPLSPLLANILLDDLDKELEKRGHKFVRYADDCNIYVKSKRAGERVMASVRNFLQERLKLKIN | |
| | EQKSAVDRPWKLKFLGFSMYKPKGGVILIRLASQTIDRVKAKIRGITARNKPISMDERIERLNTYLGGWIGYFALADTPSVFKD | |
| | IEGWMRRRLRMCLWKQWKRVRTRYRELRALGLPEWVVHHFANARKGPWRMAHGPMNRALGNAYWQSHGLMSLTERYHRLRQAW | |
| 2691323 | MSELLERILSNDNMNAAYKRVCANKGAGGVDGVTVEELGDYIKENWSSIREQIRRRQYKPQPVRRVEIPKPNGGVRNLGIPTVM | 498 |
| | DRVIQQGIVQVLSPMCEPLFSERSYGFRPNRSCEMAIRQLLVYLNEGCEWIVDIDLEKFFDNVPQDRLMSLVHDIINDGDTESL | |
| | IRKYLKAGVMTPQGYEETKLGTPQGGNLSPLLSNIMLNELDKELEKRGLRFTRYADDCVIAVKSESSAKRVMRTVADWIQRKLG | |
| | LKVNMTKTHITRPTKLKYLGFGFYRDNKAKEWKCRPHQDSVAKFKRKLKELTCRKTPGTVTGRIAKINQVTRGWINYYAIGSMK | |
| | TTMIEIDAHLRTRLRVIIWKQWKVPKKRQWGLQKLGIGKDLARLTSYYGDRYQWIVTKTCVARAISKEVLAKAGLVSCLDYYNE | |
| | RHALKLC | |
| 2691325 | VKPREAGMGGPSSSLAQVQTPSCEEGDSLLEKMLERENLLLALRRVEANKGAPGVDGVTVAQLRSYIQTHWADIRQQLLAGTYK | 499 |
| | PQPVRRVEIPKPGGGVRGLGIPTVIDRFIQQALLQVLNPIFDPEFSDNSFGFRPGRSAHDAVRRAQQYIQQGYRWAVDLDLAKE | |
| | FDRVNHDKLMARVARKVKDKRVLKLIRAYLNAGIMADGVVVRNEEGTPQGGPLSPLLANIMLDDEDKELKKRGHRFVRYADDCN | |
| | VYVKSRRAGERVMASLTRYLEGTLKLQVNQEKSAVDRPWKLKFLGFSFLPDKLATIRLAPKTLERFKERVRQITSRSRSMPIAE | |
| | RIRQLNAYIMGWVAYYRLAEMKRHCERFDEWIRRRLRMCIWKQWKRVRTRYRELRALGQPEWVVHMTANSRRGPWFMARMLNQA | |
| | MDKTYFESTGTAQSSGEIPYTSWCFMNRRMRTRTYGGVRGRGLGAPSYSIGGTSTRCIVAHRIVVTFGQCAPSGFTEVSGGR | |
| 2691327 | MEQLGGDPRYCLMEQVVERANMTKALRRVERNGGAAGVDGMEPEALRSYLKEHWPRIKEELLAGTYRPMPVRRVEIPKPGGGVR | 500 |
| | LLGIPTVLDRLIQQALLQVLTPIFDPGFSPHSYDFRPGRSAHQAVEQARRYVAQGYRHVVDLDLAQFFDRVNHDLLMARVARKV | |
| | KDKRVLKLIRAYLQAGVMINGCCVRTEEGTPQGGPLSPLLANIILDDLDKELERRGHKFVRYADDSNVYVRSCRAGQRVFESLK | |
| | RFLEQRLKLRINEEKSAVDYAWRRGILGFSFTWEKEPRIRLAPQTVKRFKQRIRWLTKRSRSVNMAERLARLNEYLKGWMGYER | |
| | LIETPSTLQALDEWIRRRLRMCLLKQWKRPRTRRRNLVALGIPEDWARHISGSRKGCWRLANTPQVNKALGLAYWRSQGLVSLV | |
| | DRYRELRSAS | |
| 2691329 | VRSREGRRQPKTLNRNCPREEVVKPRGIVGEPSPSPAQSGTSPRGDQGDSLMEKVVARDNMLAALKRVEQNKGAPGVDGIPTEN | 501 |
| | LREQIRAEWPRIREELLAGTYRPQPVRRVEIPKPGGGKRMLGIPTVMDRLIQQALLQVLTPIFDPEFSEASYGFRPGRRAHDAV | |
| | KKARQYVEEGYEWGVDMDLEKFFDRVNHDVLMARVARKVTDKRVLKVIRRYLQAGIVVNGVVMEREEGTPQGGPLSPLLANILL | |
| | DDLDKELEKRGHKFVRYADDANIYVRSRRAGERVLASVRKFLQERLKLKLNEQKSTVDRPWKLKFLGFSMYKPKAGEIRIRLAR | |
| | QSIDRVKTKIRALTARNTPIAMEERIRRLNTYLGGWIGYFALAETPSIFEEIDGWIRRRLRMCLWKQWKRVRTRYRELRALGLP | |
| | EWVVHPFANARKGPWRMAHGPMNRALGNAYWQAQGLMSLTERYRRLRQAW | |
| 2691331 | MEKQSMIINNILSNDNLNHAYKQVVKNKGAAGIDGMECEALFSHLRINGAQLKESIRNQAYNPLPVKRVEISKEDGSKRNLGIP | 502 |
| | TVTDRLIQQAVAQVLTPIYEKVFHRNSYGFRPGKSAQQAVLKAVEYMNGGYNWVVDIDLEKFFDTVNHNKLISILNKEIKDGKV | |
| | LSLIRKFLVSGVMVGEHVEETEIGTPQGGNLSPLLANIILNELDWELEKRKLKFVRYADDCIILVRSQKAAQRVMDSISKYIES | |
| | KLLLKVNRKKSKIGRPIEIKYLGFTFYNQFKAKKYKAKAHEKSVQKVVRKLKQLTSRRWGVSNSVKAQKIAEVLRGWINYFKIG | |
| | SILTITRKLDTMLRYRFRMCIWKHWKTPQNKYKNLVKLGVEPRNARRAAWSHGYARICRTEAVCYAMSNARLVKFGLLSAEAYF | |
| | VKVSS | |
| 2691333 | MHEPTLLSQIIHPANLNQAYKQVMKNKGAPGIDDMPITALKAHLALHKETLVHQLQRREYKPQPVKRVEIPKASGGVRLLGIPT | 503 |
| | VTDRFIQQAIAQVLTPIFDKQFHDNSYGFRPKRYAEMAILQALENMNEGYEWLVDIDLERFFDTVHHDRLMNIVARTVTDGDVI | |
| | SLVRKFLVSGVMVQDEYQETIIGTPQGGNLSPLLSNIMLHELDKELANRDLRFVRYADDCLIFVKSDMAARRVMRSVSKFIEEK | |
| | LGLIVNVTKSKVRRPEDEETKFLGFGFYYDWNDRLYKARPHKTSLQAIKFKLKCLTRRNWGVPTKSRVERINSLLRGWVNYFKI | |
| | GKMKKHLIQLDANIRVRLRMCIWKQWKTPKNRRKNLIKLGMNRYEAYKYSYTSKSYARVAYSWILTTTITNQRLTKFGLISATE | |
| | HYSNIHV | |
| 2691335 | MLDRDNLRLAYKRVVRNGGAPGVDGVTVAALQSYLNTHWDRVKNELLAGTYRPAPVRRVEIPKPGGGVRLLGIPTVMDRFLQQA | 504 |
| | LLQVMNPIFDAHFSWYSYGFRPGKRAHDAVMQAQRYIRDGYRWVVDLDLEKFFDRVNHDMLMARVARKVKDKRVLKLIRAYLNA | |
| | GVMINGVCQRTEEGTPQGGPLSPLLANILLDDLDKELTRRGLRFVRYADDCNIFVASKRAGERVMESAIRFLEGKLKLKVNRDK | |
| | SAVDRPWKRKFLGFSFLSDKEATIRLAPKTISRFKERVREITSRSRPIPMEERIRRLNQYTMGWVSYFRLASMKNHCERFDQWI | |
| | RRRLRMCLWKQWKRVRTRIRELRALGVPDWACFAMANSRRGPWEMSRNINNALPTSYWEAKGLKSMLTRYMVLRQPFGTAWCGP | |
| | ACQVV | |
| 2691337 | VHTHEDRIALSSPPNQADLQIHTGTLSPYTSHWGYSFLDPSIQEAPLLGQSEGQFLPPNLFSYPSGTLSSQEMPDPSPSTPELK | 505 |
| | VLPNTLKYVFLGPNETLPVIISSKLIMEQECKLTKVLLEHKAAIGWSLSDLSGISPSYCTHRIYTEEEARPSREMQRRLNPNMR | |
| | EVVKNEIIKWLDAGIIYPISDSPWVSPIHVVPKKAGLTIRVNDRGESIPTRTPTSWRVCVDYRKLNAATKKDHFPLPFIDQILE | |
| | RLAGNKFFCFLDGFSGYNQVAIDPRDQEKTTFTCPFGTFTFRRMPFGLCNAPATFQRCMLAIFHDMVGDFLEVEMDDFSVFGPS | |
| | FDECLANLTRILRRCIDVNLVLSWEKSHFMVHEGIVLGHIISDRGIEVDRAKLDIIADLPVPGSVKQIRSFLGHVGFYRRFIKD | |
| | FSKISRPLTHLLSTDVPFDMGPDCIQAFTQLRNAVTKAPVLQPPDWSLNFELMCDASDLAVGAILGQRRDRHPFVVYYASKTLD | |
| | EAQRNYTTTEKELLAVVFGLEKFRSYLLGSHVIVFTDHAAIRHLMHKKDAKPRLIRWILLLQEFDIEIRDKVGAQNVVADHLSR | |
| | LDPDHIGLTLPINEVFPDEHLLAINVASGPWYADIANYLASGSLPRGWPKNQRERLMAEAKYYFWDNPLLFRIGADQIIRRCIP | |
| | DSEFPSVLSFCHDQACGGHFSGRKTAAKVLQCGFFWPSLHQDAHTYAKHCTRCQSLGSMARRDMMPLQHILAAEIFDIWGIDEM | |
| | GPFPPSNGFEFILVAVDYVSKWIEA | |
| 2691339 | ENPIKIDFFEPLEIEPLNLIDVNFVEKMDMDIDLSYEQDSLLKQNLKNLRLDHCNKEEKDAIRKLCFDYRDIFYCEQIPLSFTS | 506 |
| | EITHKIKLNDESPIYTKSYRFPEIHKKEVKDQIAKMLDQGIIQHSISPWSSPIWVVPKKLDASGRRKWRLVIDYRKLNEKSCND | |
| | RYPLPNITDILDKLGRANYFTTLDLASGFHQIQVDPDDIPKTAFSTEGGHYEFKRMPFGLKNAPSTFQRVMDNILRGLHNEICL | |
| | VYLDDIIIFSTSLDEHIQRLKSVFDRLRKSNFKLQLDKSEFLQKTVQYLGHIITPQGVKPNPDKVSTIKRFPIPRTQKDIKSFL | |
| | GLLGYYRRFIKDFAKITKPLTKCLKKNAKVTHDQNFIDAFNTCKEILVNDPILQYPDFSKPFILTTDASDVALGAILSQGTLPN | |
| | DRPVAYASRTLNETESKYSTIEKELLAIVWACKHFRPYLYGRKFTIYTDHRPLTWLFSLKEPNSKLVRWRLKLEEFEYDIIYKK | |
| | GKLNTNADCLSRVILNAIDNESMLNNPGDIDQDILDTLQNVTQNLQTLQDFQKPSTSNINDKINIISDIQIKPPDNSQNDNTNT | |
| | STQHSTANETINDGIKIFDEIINNKTNQILVFPNVIYKLDIKRETYENHKIVTVKIPIINNEQMILQFLKENTDPKHVYCMYFQ | |
| | SDELYNDFCTVYLKHFSEKGPKLIRCLKLVNTVADKEEQILLIKNHHGSKINHRGINETLEKLKFNYYWKNMKSTVSNFINACD | |
| | ICQRA | |
| 2691341 | MIAMDDEEWGEEDIREFTKLVENSEKAWEPASEKLEVINLGNKQEKKELKIGTLVTTEERNKLVSLLHEYADVFAWTYADMPGL | 507 |
| | DTDIVVHKIPLIEGSKPVKQKTRRMRPDILLKVKSEIQKQWDAGFLDVVQYPQWVANVVVVPKKDGKIRVCVDYRDLNKASPKD | |
| | DFPLPHIDILVDNAARNATYSFMDGFSGYNQIRMAEEDKEKTTFVTPWGTFCYKVMPFGLKNAGATYQRAMVTLFHDMMHREVE | |
| | VYVDDILAKSKDEEDHVQVLRRLFERLRKYQLKLNPAKCSFGVKTGKLLGFIVSGRGIEVDPDKAKAIQEMPAPKTEKKVRSFL | |
| | GRLNYIARFISQLTVTCEPIFRLLRKKNPGVWDNDCQEAFDKIKRYLQNPPLLVPPTPGRPLILYLTVTETAMGCVLGQHDESG | |
| | RKEQAIYYLSKKENDCESRYTTIERLCCALVWSAKRLRQYMLYYTTWLISKLDPLKYICEKPYLSSRIARWQVLLAEYDIVEMT | |
| | RKAVKGSIIADHLADHAMENYEPLNFDLPDEDVIVIENEGGESDQWTLYFDGAVNVSGNGAGAVVISPENKQYPVSTRLLFECT | |
| | NNTAEYEACIIGLEAALELKAKKLEVFGDSLLIICQVKGEWQTKDEKLKLYQNYLLRLANEFEEIKFTHISRDKNQFADALATL | |
| | ASMTQVNAKSKIQPIDIEVRSFQAHCCLIEDSLDGKPWYNDIKKFLQHREYPQGISKTDEKTLRRMTMNFYLDGEILYKRSEDG | |
| | TLLRCLNENEIEQALKEVHEGICAAHANGHTMAKQMQRSGYSG | |
| 2691343 | MDIPVRPVRQRRRKENEERRLVVKEETQKLLSAGHIREIQYPEWLANVVLVKKANGKWRMCVDFTDLNKASPKDSYPLSSIDAL | 508 |
| | VDSASGCEMLSFLDAFSGYNQIKMHPRDEGKTTFMMETCSYCYKVMPFGLKNAGATYQRLMDKVLAPMLGRNVQAYVDDMVVAS | |
| | HDRRRHTADLEELFVTISKYRLKLNPKKCVFGVEAGKFLGFMLTKRGIEANPDKCAAIIAMRSPTSVKEVQQLTGRMTALSRFV | |
| | SAGGEKGYHYFQCLKRNSRFAWTDECEAAFVKLKEYLATSPVLCKPVASVPLRLYFAVTERAISSVLVQERNQVQRPIYFVIKA | |
| | LQGPETRYQSLEKAALAVVFSARRLRHYFHSFTIVVMINLPIQKVLQKPDVAGRMVRWAVELSEFDIQYEPRGSIKGQVYADFV | |
| | AELSPGGEQEVESGSQWLLSVDGSSNQQGSGAGIVLEGPNGVLIEQALRFAFKASNNQDEYEALIAGMLLAKEMGARNLLVKSD | |
| | SQLITGKVLGEFQAKDPQMAAYLRYVQLLKGAFSALELVHVPREQNARA | |
| 2691345 | MGSFLLVPEADYNLLGRDLMIELGINLEVRNKEIAVKLYALTAEDEARINPEVWYTPESVGKLDITPFEITIMNPEIPIRVKQY | 509 |
| | PMSQEGKKGLQSVIQRLLKQGLLEPCMSPYNTPILPIKKPDGSYRLVQDLREVNKRTLTRFPVVANPYTLLSQLSPELKWYSVI | |
| | DLKDAFWACPLKEECRNYFAFEWEDPETHPKQQFRWTVLPQGFTESSNLFGQALDQVLSTYELNPEVTLVQYVDDLLLAGENQE | |
| | AVREESIKLLNFLTLKGLKVSKSKLQFTEEEVKYLGHWLSKGIKKLDPERVKGILSLQAPKSKRQVRQLLGLLGYCRQWIENYS | |
| | KKVKFLYQKLVREGLIKWSQTDKKCFEDLKADLVNAPVLSLPDTKKPFYLFVNTEEGMAYGVLTQKWADRKKLIGYFSKLLDPV | |
| | SRGWPTCLQAIVATALLVEEAGKVTLGGELKVYTPHNIRGVLQQKAKKWITDARLLKYEGILIHSPKLEIETTSLQNPAQFLYG | |
| | EPSEELTHDCVRLIESQTKIREDLEEEELPSGEKLFVDGSLRVVNGKRKSVYAIIDGNTSEVKESGPLDTTWSAQACELFAVLK | |
| | ALQLLKGKVGSIFTDSRYAYGVVNTFGKIWEERGLINTQGKNLVHETLIRQTLEALRGPRQVAVVHVKGHQKGASVQIRGNNLA | |
| | DEEAKRAALLIVKETEKPREATGNSKSFTARGIEK | |
| 2691347 | VGGQSVDFLLDTGATYSVLTEAPGPLSSRSASVMGLSGRAKRYYFSYSLSCNWDSVLFSHEFLIVPESPSPLLGRDILSKVHAS | 510 |
| | VFMNMEPSLSLPLVEQNVNPRVWADGKSVGRAQNAIPVVVKLKDPHVFPHKKQYPLKPEVKEGLKPIIENLKEQGLLIPCNSPC | |
| | NTPILGIKKSNGKWRLVQDLQIINEAVVPLHPVVPNPYTLLSEIPERAKYFSVIDLKDAFYSVPLAEESQFLFAFEDPTQPASQ | |
| | LTWTVLPQGERDSPHLFGQSLSRDLQNENSSEAVVLQYVDDILLCAETEEACSRASEDELNFLAGCGYKASREKAQLCQQSVRY | |
| | LGLIISEGTRAIGPERIKPILNHPLPMTLRQLRGELGITGYCRIWIPGYGELARPLYKLIAETQQAQTDKLVWSPETQKAFKVL | |
| | QTALLQAPALSLPTGSEFNLFVTERKGVALGVLTQPRGPHQQPIAYLSRELDVVSRGWPHCLRVIGAAALLAPEALKIINGRNL | |
| | TVLTSHDVSGILNSKVNIWMTDSRLLKYQSLLLEGPVTKLKVCGNLNPATFLPEKENETPDHDCSQFLTLNYAAREDLKDTPLD | |
| | NPDMEIFTDGSSFVRDGKRKAGYAVVTAEQVLEAKSLPQGTSAQLAELVALTRALELSKGQRMGLIIYVSLLLLTPKILSLPLD | |
| | PQDNVFLSWAHSYAAFHNRSNCWVCGALPSSSVEGFPWWTSPLQGKDFLQVCEYLRQQSHAMPLLHLMTSTNPKMDWCNTL | |
| 2691349 | MLSEEALEVLKTVPPCVWSQGKCDVGRMVNAKPVHVRPKTNYRPNIRQYPLKEDAKVGIKPVIEEMLKAGILRECPDASCNTPI | 511 |
| | FPVKKADGQSWRMIQDLRAVNDAVQSTAPTVPDPHTLMNGLKPNMAWFSVIDLSNAFFSIPVEKDSQGWFGFTHEGTKYTYTRL | |
| | PQGYVDSPTIFSREIANCLATLQIPDTSQILVYVDDILVASPTQEESTKVTLQVLTHLAKTGNKVSRQKLQFVKEEVIFLGHNL | |
| | NREGRRIHSERKQTVLNEPKPTTKKQMMQFLGLTNYCRAWIPDYAGMTQPLLDCIYSDNDMKMSGKIEWTQEAEAAFESTKQCL | |
| | VGSSVLALPDYEKPFVQTVDCKKGFMTSVLAQKHGTKLRPVAYYSKRLDPVAQALPVCVQAVCAAAMAVHCTAEIVLFHPLTLM | |
| | VPHAVTMLLHDTKMAFLSPARYLALTATLMSQPHIVIKRCNILNPATLIPTAEDGEPHCCKEETDRICKPRPDLKDIQLLSGET | |
| | WFVDGSCSKSITGQNQTGFAVVSHSQVIKAGRLPHTYSAQAAELVALTEACKAGVGKDVTMWTDSQYAHSTVHIFAAQWARRGM | |
| | KTSTGKPVTHAQLLTDLLKAVLLPKSIAICKCAAHTSGKDAVTLGNAHADKVAKLAAMGEYGFHILLQKGESVSQPIPLVILRD | |
| | MQNSAPDREKKKWLTDGATTDPEGTFRINNKIVLPVSLYKTAAHLSHGPCHVSTGGMVTIINEHFHTYNYITFSKNFCRACVVC | |
| | CRHNAQGNERPQRGK | |
| 2691351 | MVMALLAATSAPTVPTVPVELSDISETLWTTISTETGLLKSAEPVYITYHTDIPLPRKMQYPLPKEAIEGIQPVLQQLLTQGII | 512 |
| | KEATSPCNTPILPVKKPKPGSYGKPVYRFVHDLRLINAIVIPRAPVIPNPATVLTAIPPEATYFTVIDLSSAFFSNPVHPDSQF | |
| | LFAFTFDGRQYIWTRLPQGYRESPTIFSQYLKWDLDSIRCAMGSTLIQYVDDLLLCSTAKDACVQDTRILLFALAEKGHKVVLS | |
| | KLQFAQSEVEYLWHRISYVSTALTQDRIESVLSKPRPTTKKQLHQLLGASGYCRAWIPAYVELVHPLTDLLLDSIPEPLPWTTL | |
| | HDNALTALKQALTSAPALGRPDYAKPFTLFCHECYGTASGVLTQPFGPGQRMVAYFSVLLDPCAQGLPPCLRAVGAVAMLVETA | |
| | LGLTLGHPLLVQVPHAVAALLNRGSTQHLTAARLTKYELALLANQTITLHRCEVLNPATLLPLPEDGMPHNCLHLMDLISTPRP | |
| | DLSDVPLQNPDLQLFCDGSAHQDEFGALRVGYAIVILMTTLEAHSLPTQKSAQAAELVALVRACQLAENKTVAIYTDSKYAFSV | |
| | CHSTGQLWKHHGFLTSCGSQISHAALISALLEAIQLPAAVSVTHCDFVCVKIHRRKHCLEPWWSAPAQVLLTTPTAVKIEGIDR | |
| | WVHCSHCKKVPAPTSPTTAITTDALT | |
| 2691353 | VFAWDAYDAPGVDPSLICHHLNVNPSSTPKKQPPRRPSKEHASAVKDEVAKLKRAGAIKEVFYPEWLANTVVVKKKTGKWRVCV | 513 |
| | DFTDLNKACPKDPFPLPRIDRLVDATVGHPRMSFLDAFQGYHQIPLALEDQEKTAFMTPIGNYHYKVMPFGLKNAGSTYQRMMT | |
| | RMFEPQLGKIIEVYIDDMVVKSRRASEHVKDLGTIFTILREHRLRLNASKCSFGVGSGKFLGYMVTHRGIEVSPDQIKAINSLQ | |
| | APRNPKEVQKLTGMIAALNRFISRSADRCRPFYLLINKWKEFAWSEDCVQAFQQLKDYLSRPPIMSSPEADEVLFAYIAVAPHA | |
| | VSLVLIRDDNGVQRPVYYVSKSLHEAEVRYLPLEKAILAIVHATRKLPHYFQAHTVIVLTQLPLRAVLRSADYTGRIAMWSALL | |
| | GAFDIKYMPRSSIKGQVLADLVAEFAEPSIETITEKRDMDGKSVGAISTGRNTHWKVYVDGAANQRGSGVGIVLISPDGAAIEK | |
| | SLRLGFSATNNEAEYEALLQGMTMVQRLGGKMIEAFSDSRLVVGQVMGELEARDTRMQEYLGQVKRLQTNFESFNLTHISRSVN | |
| | THADSLATLATSSAHNLPRVILVEDLAQASPISRGPARVHQIRKSHSWMDPIKNFLQNDVLPEENWKPRRYEGMLLGFGCQRTT | |
| | NYIGAPILDRTYSAYTQKNPNLCLRSYTRGFVEVIPEGDRWHTGHLPRGTGGRTCRERPKNTLRSAISAKDSPRISTNPEEFST | |
| | PSRVHGRSHNGALI | |
| 2691355 | VLIRDTSITTNPLPARYHDFSDVFEKKNADRLPEHRLYDCPIDLQEGAHPPFGPIYGLAEPELEALREYLKENLAKGFIQPSKS | 514 |
| | PAGAPILFVKKKDGSLRLCVDYRGLNRVTVRNRYPLPLIPELLDRLRTGRVFSKIDLRGAYNLVRIKPGDEWKTAFRTRYGHFE | |
| | YKVMPFGLTNAPAVFQHMMNHIFREYLDHFVVIYLDDILVFSPSMEEHTRHVRLILTKLREHGLYAKSEKCEFDRTSVEFLGYV | |
| | ISPAGITMDPRKVATIHNWPIPTRLKEVQSFLGFANFYRRFIDRFSTIVQPLISLTRKDVPFVWTATTQHAFDALKQAFMSAPV | |
| | LVHPNPAKPFQVETDASDFALGAILSQLDDDGTLHPVAYYSRKFLASEINYPVYDKELAAIISAFAEWRPYLVGAQHRIQVVTD | |
| | HKNLLYFASSRTLNRRQARWSIFLADYDFEIIFRPGIQHGKADALSRRPDLALCPGDDAYTQQSCSLLKPDQLQLSATFMLHDD | |
| | SLLQEIAQATKTDTFATEILKRLQDSSPATKRADLHHFTTHDGLLYRNHLLYVPEGPCRTQVLQTCHDDPLAGHFGVAKTLELV | |
| | SRGFWWPQPWKLVKEFVKTCDVCARSKAVHHRPYGLLHPLPIPNRPWASISMDFITDLPPVNGVDTV | |
| 2691357 | VSAPDPPVPHADKIIWETQIPVWVDQWPLTQEKLKAASMLVQEQLAADHVEPSTSPWNTPIFVIKKKSGKWRLLQDLRAINRVM | 515 |
| | RPMGALQPGIPTPVAIPKDYYKMVIDLKDCFFTIPLHPEDRPYFAFSLPRINFQGPMQRFQWKVLPQGMANSPSLCQKYVAQAV | |
| | DPVRKRWPQIYILHYMDDLLIAAPDTHNLNGCYLDLYKALTTLGLQIAPDKVQTQDPYTYLGLQLQGNQIQTPKIQLRVDHLHT | |
| | LNDFQKLMGDIQWLRPYLKLPTCVLLPLNDILKGDSHPTSPRKLTPEAQQALNQVTKAITQQFVTQISYNLPLILVILHTPHTP | |
| | TGIFWQRDPHFKNKGCPLFWVHLPTTSSKVITVYPAQVASVIVKGRLLSRQHFGKDPDKILIPYTKDQTQFLLQTEDEWSIALS | |
| | GFLGTIDNHLPSDPLLHFAQLHPFIFPKITQKAPIPQALTVFTDGSSNGCAVFVVGNQPTTFETAYQSAQLVELFAIAQVFITF | |
| | LDKPVNIYTDSAYIAHSVPLLEISPAIKASSNAAFLFHQLQQLIQTRRHPFFLGHIRAHSDLPGPLTQGNAQADAATRSIFPIF | |
| | AGSIQRATELHTLHHLNSQSLRLFCKITRQQAREIVKACPACT | |
| 2691359 | VSAPDPPVPHADKIIWETQIPVWVDQWPLTQEKLKAASMLVQEQLAADHVEPSTSPWNTPIFVIKKKSGKWRLLQDLRAINRVM | 516 |
| | RPMGALQPGIPTPVAIPKDYYKMVIDLKDCFFTIPLHPEDRPYFAFSLPRINFQGPMQRFQWKVLPQGMANSPSLCQKYVAQAV | |
| | DPVRKRWPQIYILHYMDDLLIATPDTHNLNGCYLDLYKALTTLGLQIAPDKVQTQDPYTYLGLQLQGNQIQTPKIQLRVDHLHT | |
| | LNDFQKLMGDIQWLRPYLKLPTCVLLPLNDILKGDSHPTSPRKLTPEAQQALNQVTKAITQQFVTQISYNLPLILVILHTPHTP | |
| | TGIFWQRDPHFKNKGCPLFWVHLPTTSSKVITVYPAQVASVIVKGRLLSRQHFGKDPDKILIPYTKDQTQFLLQTEDEWSIALS | |
| | GFLGTIDNHLPSDPLLHFAQLHPFIFPKITQKAPIPQALTVFTDGSSNGCAVFVVGNQPTTFETAYQSAQLVELFAIAQVFITF | |
| | LDKPVNIYTDSAYIAHSVPLLEISPAIKASSNAAFLFHQLQQLIQTRRHPFFLGHIRAHSDLPGPLTQGNAQAD | |
| 2691361 | VEEAAPIQGNNRDQQAANAMEREIGGKTFKLGRLLSQEKQEGVAEVISRHLDAFAWSASDMPGIDPDFLCHHLSMDVAVRPVRQ | 517 |
| | RRRKYNEERQLVVKEETQKLLSAGHIRVIQYPEWLANVVLVKKANGKWRMCVDFTDLNKACPKDSCPLPSIDALVDSASSCKVL | |
| | SFLDAFSGYNQIKMHPRDESKTAFMIETCSYCYKVMPFGLKNTGTTYQRLMDKVLAPMLGRNVYAYVDDMVVASQDREHHVADL | |
| | EELFVTISKYRLKLNPEKCVFGVDAGKFLGFMLTERGKEANPNKCAVIIAMRSPTSVKEVQQLTGRMAALSRFVSAGGQKGHPY | |
| | FQCLKRNSRFAWTDECEAAFIKLKEYLATPPVLCKSVTGVPLRLYFTVTERAISSVLVQEQDHSQKPISFVSKALQGAETRYQA | |
| | LEKAALAVVFSARRLRHYFHSFTVVVIANLPIQKVLQKPDVAGRMVRWAVELSEFDIQYEPRGSIKGQVYADFVAELSPGGEQE | |
| | VEVGSQWLLSVDGSSNQQGSGAGIVLEGPNDVLIEQALRFAFKASNNQAEDEALIAGMLLAKEMVAQNLLVKSDSQLITGQVSG | |
| | EFQAKDPQMAAYLKYVQLLKG | |
| 2691363 | VYRKQFKIPEAHQTFIEQTLDEWLKLGVVKRSNSLYNSPLFCVPKKQGRGLRIVQDFRELNNHSHIDKYSMKEITECIGDIGRA | 518 |
| | NSSIFSTLDLTSGFWQMKLDEASQPLTAFTIPGQGQFQWITSPMGLLGCPASFQRLMETVLRGIKNVLVYIDDLLIHTATHEEH | |
| | LIVLEQVFERLHKNHLKINLEKCVFGNPEVSYLGYTLTPNGIKPGRNKLQAIKDAQPPTNIKMVRSFVGLCNFFRTHIKDFAII | |
| | AAPLFKVTRKDSNYKSGQLPPDALHAFRVLQLQLTSEPVMAYPRSDRQYALITDAATGTAETPGGLGAILTQIDKSGNHFAISF | |
| | ASRQLKDHEKNYSPFLLEAAAAVWGMDVFNEYLRGKQFILFTDHKPLEKLGHLHTKTLNRLQTALLEHDFVIQYKKGAIMPADY | |
| | LSRLPTLQVQNVEATVAAFDPFQPDLSKLQVPFQVNTAADVIAAFDPFQPNLKELQFQDTDLQAIYLFLKNGQWLPHLTKRQIN | |
| | SLSALSQKVFFDKNKLAWIRLDDYKYPRTALWLPEFYRKEALCESHNQIFAGHNAAQKTYLKLTSSYFWPNVYSHVLQHTQTCF | |
| | RCQQRKSSRAKQPPLAPLPIPDLPNVRIHADLFGPMLGVEKKFAYVLCITDAFTKYAMVTKVDNKDAETVARAIFDNWFCKFGI | |
| | PAQIHTDGGKEFVNKLSAELFELLNVQHSKTSPYHPQCNAQVEVENKTVKKYLASYVDKTTLNWNDFLPALMLAYNTSYHSTIA | |
| | TTPFELLFGVK | |
| 2691365 | MKEDHRKIEELVPRKFLKWRKVFGKVESERMPTRKVWDHAIDLKEMFKPQKGRIYPLSKNKREEVQNFVEDQLRKGYIRPSKSP | 519 |
| | QTLPVFFVGKKDGSKWMVMDYHNLNNQTVKNNYLLPLITELIDNMGSKKVFTKMDLRWGFNNMRIKEGDEWKGVFTMHIGSFEP | |
| | IVMFFGMTNLPATFQAMINEILRDLINKGKVAAFVDDVLVGTETEKGHDKIVEEILRRLEENDLYVKPEKCIWKVWKIGFLGVV | |
| | MEPNGIEMEEEKVDRVLSWPELENVKDIRKFLGLANYYRRFIKDFARVARPINMLMRKDVKWQWGVEQQKAFDKLKRVFTTKLV | |
| | LAAPDLDKEFRVEADVSNYATRGVLSMKCSDEMWRPIAFISKSLSDTERNYEIHDKKMLAVVRCLEAWRYFLEGATTKFEIWTD | |
| | HKNLEYFMKAQKLNRRQARWALYLSRFNFMLKHVLGSKMGKADSLSRRPDWEVGVEKDNKDQKLVKPEWLEVRKTEMVEIIVDG | |
| | VDLLEEVRKSKVRDDEVVKVVEEMKKAGVKILRDEEWREADGVMYKKEKVYVPKDNKLRAEIIRLHHDMPVGGHGRQWKMVELV | |
| | TQNFWWPGITKEVKRYVEGCDACQHNKNRTEQPAGKLMPNSIPEKPWTHISVDFITKLPLVQGYDSILVVVDWLTKMVHFIPTM | |
| | EKTSAEGLARLFRDNVWKLHGLLES | |
| 2691367 | MKEDHRKIEELVPRKFLKWRKVFGKVESERMPTRKVWDHAIDLKEMFKPQKGRIYPLSKNKREEVQNFVEDQLRKGYIRPSKSP | 520 |
| | QTLPVFFVGKKDGSKWMVMDYHNLNNQTVKNNYLLPLITELIDNMGSKKVFTKMDLRWGFNNMRIKEGDEWKGVFTMHIGSFEP | |
| | IVMFFGMTNLPATFQAMINEILRDLINKGKVAAFVDDVLVGTETEKGHDKIVEEILRRLEENDLYVKPEKCIWKVWKIGFLGVV | |
| | MEPNGIEMEEEKVDRVLSWPELENVKDIRKFLGLANYYRRFIKDFARVARPINMLMRKDVKWQWGVEQQKAFDKLKRVFTTKLV | |
| | LAAPDLDKEFRVEADVSNYATRGVLSMKCSDEMWRPIAFISKSLSDTERNYEIHDKKMLAVVRCLEAWRYFLEGATTKFEIWTD | |
| | HKNLEYFMKAQKLNRRQARWALYLSRFNFMLKHVLGSKMGKADSLSRRPDWEVGVEKDNKDQKLVKPEWLEVRKTEMVEIIVDG | |
| | VDLLEEVRKSKVRDDEVVKVVEEMKKAGVKILRDEEWREADGVMYKKEKVYVPKDNKLRAEIIRLHHDMPVGGHGRQWKMVELV | |
| | TQNFWWPGITKEVKRYVEGCDACQHNKNRTEQPAGKLMPNSIPEKPWTHISVDFITKLPLVQGYDSILVVVDWLTKMVHFIPTM | |
| | EKTSAEGLARLFRDNVWKLHGLLESIISDRGPQFAAGLIRELNGILGIASKLLTVFHPQTNGQTERVNQELEQYLRMFINYRQE | |
| | QWPEWLGTAEFAYNNKVHSST | |
| 2691369 | MVKIRGDLSSEMKQQLYNPLKEFKDIFAWSYKDMPGLDFEIVQHRLPLKPECHPIKQRLRRMKPEVSLKIKEEVEKQFNAGFLA | 521 |
| | VAQYPQWVANVVPVPKKDGSVRMCVDYRDLNRASPKDDFPLPHIDILVDNTATSSLYSFMDGFSGYNQIKMTPEDMEKITFITL | |
| | WGTFCYKVMSFGLKNAGATYQRAMMALFHDMMHKEIEVYVDDMIAKSESEEEHLVNLKKLFERLRKYKLRLNPSKCTFGVKSGK | |
| | LLGFIVSQKGIEVDPDKVRAILEMPHPCTEKQVRGFLGRLNYIARFISQLTATCEPIFKLLRKNQVVKWNENCQNAFDKIKEYL | |
| | KEPPILHPPVPGRPLILYLTVLDGSMGCVLGQHDGTGKKEHVIYYLSKKFTDCEQRYSSLEKTCCVLAWTAHRLRQYMLSHTTW | |
| | LVSKMDPIKYIFEKPALTGRIARWQMLLSEFNIVYVIQKAIKGSALAEYLAQQPIDDYQPMQPEFPDEDIMTLFVSEDDGNERK | |
| | WVLFFDGSSNALGHGIGAVLISPEKQYIPMTARLCFDCTNNIAEYEACAMGIRAAIEYKARRLNVYGDSALVIHQVKGEWETRD | |
| | QKLIPYKAYIRGLVEYFDEIEFHHISREDNQLADALATLSSMFAISQEEELPMIKMQSHENPVYCNFIEEEVDGKPWYFDIKRY | |
| | LQNREYPDSASENDKRMLRRLASSFILDGDVLYK | |
| 2691371 | MVKIRGDLSSEMKQQLYNPLKEFKDIFAWSYKDMPGLDFEIVQHRLPLKPECHPIKQRLRRMKPEVSLKIKEEVEKQFNAGFLA | 522 |
| | VAQYPQWVANVVPVPKKDGSVRMCVDYRDLNRASPKDDFPLPHIDTLVDNTATSSLYSFMDGFSGYNQIKMTPEDMEKITFITL | |
| | WGTFCYKVMSFGLKNAGATYQRAMMALFHDMMHKEIEVYVDDMIAKSESEEEHLVNLKKLFERLRKYKLRLNPSKCTFGVKSGK | |
| | LLGFIVSQKGIEVDPDKVRAILEMPHPCTEKQVRGFLGRLNYIARFISQLTATCEPIFKLLRKNQVVKWNENCQNAFDKIKEYL | |
| | KEPPILHPPVPGRPLILYLTVLDGSMGCVLGQHDGTGKKEHVIYYLSKKFTDCEQRYSSLEKTCCVLAWTAHRIRQYMLSHTTW | |
| | LVSKMDPIKYIFEKPALTGRIARWQMLLSEFNIVYVIQKAIKGSALAEYLAQQPIDDYQPMQPEFPDEDIMTLFVSEDDGNERK | |
| | WILFFDGSSNALGHGIGAVLISPEKQYIPMTARLCFDCTNNIAEYEACAMGIRAAIEYKARRLNVYGDSALVIHQVKGEWETRD | |
| | QKLIPYKAYIRGLVEYFDEIEFHHISREDNQLADALATLSSMFAISQEEELPMIKMQSHENPVYCNFIEEEVDGKPWYFDIKRY | |
| | LQNREYPDSASENDKRMLRRLASSFILDGDVLYK | |
| 2691373 | MKYPYSNVYSITCYDQIDKCVQQVCDFDCEDGLSITLSYGYDFTKIEEMERHVCVPQNVHESVLALQALQTVPHGNLFVDLVLS | 523 |
| | HKKLLPSILQAPELELKPLPDNLKYVFIGDNNTLPVIIAKGLINIQEEKLVKLLCDHKTAIGWTLADIKGISPSMCMHHILLED | |
| | NAKPTREMQRRLNPPMMEVVKAEILKLLDAGVIYPITDSKWVAPIHVVPKKTGITLVKNKNDELIPTRISSGWRMCVDYRKLNL | |
| | STRKDHFPLPFMDQMLERLAGKSFYCFLDGYSGYNQIVINPEDQEKTTFTCPFGTYAYRRMPFGLCNAPATFQRCMMSIFSDYV | |
| | ERIIEVEMDDFTVYGDSFDKCLENLSLILKRCIETNLVLNYEKCYFMVEQGIVLGHVVSSRGLEVDKAKIDVISSLPYPSCVRE | |
| | VRSFLGHAGFYRRFIKDFSKITAPLCRLLAKEVDFVFDQACKDAHDELKRRVTSAPIIQPPNWDEPFEIMCDASDYAVGAVLGQ | |
| | RIGKNLHVIAYASRMLDGAQCNYHTTEKELFAVVFALEKFRSYLLGTKVIVFTDHAALRYLLKKKESKPRLIRWILLLQEFDLE | |
| | IKDKKGAENHVADHLSRLRTEDIQIETIRETFPDEQLYMLHSSTRPWYADLVNYLVTKEFPPGLSKPQKDKLRADAKYYFWDDP | |
| | YLWKFCADQVVRRCVP | |
| 2691375 | MPGVPRNLIEHSLNVSKTARPIKQKLRRFARDKKEAIRAEITRLLAAGFIKEVYHPDWLANPVLVRKKNNEWRMCVDYTDLNKH | 524 |
| | CPKDPFGLPRIDEVVDSTAGCELLSFLDCYSGYHQISLKEEDQIKTSFITPFGAYCYTTMSFGLKNAGATYQRAIQQCLHDEIR | |
| | DDLVEAYVDDVVVKTRDASTLIDNLDRTFKALNRYKWKLNPKKCIFGVPSGLLLGNVVSRDGIRPNPSKVKAVLDMRPPKNVKD | |
| | IQKLTGCMAALSRFISRLGEKGLPFFKLLKASEKFSWTEEADTAFNQLKTELTSPPVLTAPQPNEDLLLYIAATDRVVSTAIVV | |
| | ERDEPGHVFKVQRPVYFISEVLNESKARYPQIQKLIYAILITSRKLKHYFDGHRVLVTTSFPLGDILRNKDANGRIVKWAMELC | |
| | PFSLEFQSRTTVKSQALVDFIAEWTDLNEPSVPDVSDHWSMFFDGSLNINGAGAGILFVSPNKDKLRYVLRILFPASNNVAEYE | |
| | ACLHGIRLAVELGVKRLYVYGDS | |
| 2691377 | VPGNEDLDFRKQISPELDEEETEKLVKALGRFVDVFAKGNEPLGMCNMAEHEIPLVPNAKPVYQSLGSGAYKEQLIHRELMSKM | 525 |
| | KARGAIEVSVGPWGARVLLAQKKDGTWRFCVDYRLLNALTVNSVYPMPSIDGTLARLHGAKIFSIMDLECGYWQIPIKKEDRVK | |
| | TAFITADGVFQFICMPFGLCTAPSTFQRTMDLVLGGLRWSVCLVYLDDIIVYARNTEEHRQRLVMVLSALRKANLKLKLAKCRF | |
| | AEKEITALGHRISAEGVRPDPDKVRAVKEFPGLPTSGKRADLVKWVKSFLGLVSYYRRFFPGFAEVAKPLMDLTKDKTLFIWGA | |
| | SHQKSFDELKERLANAAVQAYPDYSLEFEIHPDACGYGVGAVLVQRQMGEERPLAFASRIMTASERNYSITEKECLALVWAVKK | |
| | FRFFIWGRPIRIVTDHHALCWLQSKKDLAGRLSRWAMQLMEYKYTIAHKDGRLHADADALSRYPLALGLGSDATDKESRDLVVN | |
| | VVTQCDRSELQEGQRSEWAYVENNHEQEKETANYTIRNGLLFRIRLWMASRGKLRCDSACRRPCGRGY | |
| 2691379 | MSLMEEVVSRENMQAAYRRVVRNRGAAGPDGMTVEDLADHCRQHWPRIREELLAGRYRPQPVRRVTIPKPGGGERALGIPTVLD | 526 |
| | RMIQQALLQVLQPVFDPRFSPDSYGFRPGRGAHDAVLRARDHVRGGRRWVVDLDLEKFFDRVNHDVLMSRVARRIEDKGVLRLI | |
| | RRFLQAGVLEGGVASPRLEGTPQGGPLSPLLSNILLDEWDKELERRGHRFVRYADDCQIYVQSQRAGERVMRSMTRFLEEVLRL | |
| | RVNRVKSAVARPWERQFLGYGFTQH | |
| 2691381 | MEIETPQRLRKLQQVLHRNAKANSKWRAWSLYADLLREDVLAHAAAKVIANKGAAGIDGTSVDDIRSSESRERFLSDLREELSS | 527 |
| | KAYKPSPVLCVRIPKKDGKTRALGIPTVKDRVVQPALVLLLEPIFEADMHAESYGYRKGKNAHQAMDSICRALYSGRHEVIDAD | |
| | ISGYFDNIDHAMLMKLVKRRVSDGSILKLIKSFLTAPVVENGTGRKNDKGTPQGGVLSPLLANLYLNGLDHQVNGKAGQGAQMV | |
| | RYADDLVILCHRGKGAELLERLDRYLATKGLSLNRRKTRRVDFTKE | |
| 2691383 | LGIPTLTDRLIQQALHQVLSPIFEAEFSESSYGFRPGRNAHQAVKAAKQYVAEGRRFVVDMDLEKFFDRVNHDVLMAKVAKKVE | 528 |
| | DGRVLKLIRRYLEAGMMAEGVVSPRTQGTPQGGPLSPLLSNILLTDLDRELERRGLAFCRYADDCNIYVRSQAAGERVLAGISR | |
| | FLSERLKLRVNEAKSAVARPWERKFLGYSLTWHKTPKLRIAPASLQRLKGKIRKMLKGASGRSVRRTIEELNPVLRGWAAYFKL | |
| | AETKSALEELDGWLRRKLRCILWRQWKRPYTRARNLMKAGLKEERAFRSAFNQRGPWWNSGASHMNAAFRNVYFDRLGLVSLLD | |
| | TTRRLQYLS | |
| 2691385 | EYKVSIAPIPEYILGIDIMSGLTLQTTVGEFRLRERCISIRAVQAIIRGHAKIEPIFLPQPRCITNTKQYRLSGGEEEFTKIVQ | 529 |
| | ELERVGIIRPAHSPYNSPIWPVRKPDGTWRMTVDYRELNKVTPPIHAAVPNIASLMDTLSREIETYHCVLDLANAFFSIPIAKE | |
| | SQDQFAFTWEGRQWTFQVLPQGYVHSPTFCHNLVASDLANWNKPSTVKMFHYIDDLMLTSDSIEALEKTVPSLITYLQEKGWAI | |
| | NPQKVQGPGLSVKFLGVVWSGKTKVLPSAIIDKIQAFPVPTKPKQLQEFLGILGYWRSFIPHLAQLLKPLYRLTKKGQVWDWGR | |
| | TEQEAFQQAKIAVKQAQALGIFDPTLPAELDVHVTQEGFGWGLWQRQGSVRIPIGFWSQIWHGAEERYSMVEKQLLATYSALQA | |
| | VEPITQTAEVIVKTTLPIQGWVKDLTHIPKTGVAQSQTVARWVAYLSQRSRLSSSPLKEELQKILGPVTYHSETPEEIVVTCPE | |
| | ESPVQEGKYPIPEDAWYTDGSSRGNPSRWRAVAYHPSTETIWFEEGDGQSSQWAELRAVWMVITQEPGNSALNICTDSWAVYRG | |
| | LTLWIAQWATQDWTIHARPIWGKDMWVDIWNVVRHRTVRAYHVSGHQPLQSPGNDEADTLARV | |
| 2691387 | MIDGLGFANSFQNPSHNSLIFISTYVNKRIMNIMVDTGSQHSFINKNCLREFDKLQSFEISPQNFFMADGMTTFQVKDTIRLNI | 530 |
| | SIGTFDTTAVAFVTTHLCTDMILGMDYLSQYDIEIKTKQNYITFNFGNEQVILSINLESSSKRHSSSNPELIRSSINQQFHTSA | |
| | LSSINNLEQIISNLANHMTNPTQQNNLKSLLFKFQSTEDTSKYTIAKTEIFHVIETETHIPPASKCYPGNPTLNAELRKIVDKL | |
| | LDAGLIANSQSPYAAPALLVKKKDNTWRLVIDYKKLNSITLKDNYPLPNMEMALQTLGCGYSYFSKFDLRSGFWQLPIDPKDRF | |
| | KTAFATPFGLFEWLVLPQGLRNSPPTFQRTMNRVLSACTEFSLVYLDDIVIFSHTFDDHLVHIEKVLSALREHNLTLAPSKCEL | |
| | AKQSIEYLGHVISSKTIAPLPDRIKSIILLPEPKTLTQANKFIGSLSWYRKFIPAFATLAAPIHSITNLSKSNRHKFHWGIDQS | |
| | KSFAALKQFLTSSPIFLDFPVDGQPIFLATDASLVGLGGVLYQEVQGEKRILYYHSELLTPAQKRYHPLELEALAIFKCVNRMK | |
| | SFLLGREIFVYTDNCPICHMLDKKVSNKRVEKISILLQEFNIRRIIHVKGEYNCLPDYLSRHPIEQDNEFLNSNYGLEFVRDDS | |
| | SSIQIAGAVVTRSKAKALPPPPTNSSQTNSQQTTSLISPTSNRDSLNELEPEPESVDMNEDLTQIKQHQLGDVRIQQVIEDLKT | |
| | NPNSSF | |
| 18266 | MSELLEKILSKDNMNTAYKRVCANKGAGGVDDVTVEELGAYVKENWESIREQIRRREYTPQPVRRVEIPKPDGGERKLGIPTVM | 545 |
| | DRVIQQGIVQIISPMCEPLFSKWSYGFRPNRSCEMAIRQLLEYLNEGYEWIVDIDLEKFFDNVPQDRLMSLVHDIIKDGDTESL | |
| | IRKYLKAGAMTPQGYKETKLGTPQGGNLSPLLSNIMLNELDKELEARGLRFTRYADDCVIAVKSESSAKRVMRTVTDWIQRKLG | |
| | LKVNMTKSQITRPQKLKYLGFGFHKDSKAKEWKCRPHQESVKKFKVRLKELTCRKKPGTVTSKIAKINQVTRGWINYYALGSMK | |
| | TAMTEIDAHLRTRLRVIIWKQWKVPKKRQWGLQKLGVSKDLARLTAYCGDSYYWVATKTCVVRAISKEVFAKSGLVSCLDYYNE | |
| | RHALKLC | |
| 18268 | MSELLERILSNDNMNAAYKRVCANKGAGGVDGVTVEELGDYIKENWSSIREQIRRRQYKPQPVRRVEIPKPNGGVRNLGIPTVM | 546 |
| | DRVIQQGIVQVLSPMCEPLFSERSYGFRPNRSCEMAIRQLLVYLNEGCEWIVDIDLEKFFDNVPQDRLMSLVHDIINDGDTESL | |
| | IRKYLKAGVMTPQGYEETKLGTPQGGNLSPLLSNIMLNELDKELEKRGLRFTRYADDCVIAVKSESSAKRVMRTVADWIQRKLG | |
| | LKVNMTKTHITRPTKLKYLGFGFYRDNKAKEWKCRPHQDSVAKFKRKLKELTCRKTPGTVTGRIAKINQVTRGWINYYAIGSMK | |
| | TTMIEIDAHLRTRLRVIIWKQWKVPKKRQWGLQKLGIGKDLARLTSYYGDRYQWIVTKTCVARAISKEVLAKAGLVSCLDYYNE | |
| | RHALKLC | |
| 21531 | MSELLEKILSKDNMNAAYKRVCANKGAGGVDDVTVEELGAYVKENWESIREQIRRRKYTPQPVRRVEIPKPDGGERKLGIPTVM | 547 |
| | DRVIQQGIVQIISPMCEPLFSKWSYGFRPNRSCEMAIRQLLEYLNEGYEWIVDIDLEKFFDNVPQDRLMSLVHDIIKDGDTESL | |
| | IRKYLKAGVMTPQGYKETKLGTPQGGNLSPLLSNIMLNELDKELEARGLRFTRYADDCVIAVKSESSAKRVMRTVTDWIQRKLG | |
| | LKVNMTKSQITRPQKLKYLGFGFHKDSKAKEWKCRPHQESVKKFKVRLKELTCRKKPGTVTSKIGKINQVTRGWINYYALGSMK | |
| | TAMTEIDAHLRTRLRVIIWKQWKVPKKRQWGLEKLGVSKELARRTAYCGDSYYWVATKTCVVRAISKEVFAKSGLVSCLDYYNE | |
| | RHALKLC | |
| 21532 | MSELLEKILSKDNMNAAYKRVCANKGAGGIDDVTIEELGDYIKENWESIREQIRHREYKPQPVRRVEIPKPNGGVRELGIPTVM | 548 |
| | DRVIQQGIVQIISPMCEPLFSEWSYGFRPNRSCEMAVRQLLVYLNEGYEWIVDIDLEKFFDNVPQDRLMSLVHDIIKDGDTESL | |
| | IRKYLKAGAMTPQGYEETNLGTPQGGNLSPLLSNIILNELDKELEGRGLRFTRYADDCVIAVRSESSAKRVMRTVADWIQRKLG | |
| | LKVNMTKTRIARPQKLKYLGFGFYKDSKAKEWKCRPHQESVKKLKDRLKELTCRKKPGTVTSKIAEINQVTRGWINYYALGSMK | |
| | TAMTEIDAHLRTRLRIIIWKQWKVPKKRQWGLQKLGIGKDTARLTAYCGDRYYWVATKTCVVRAISKDVFAKAGLVSCLDYYNE | |
| | RHALKLC | |
| 21533 | MSELLEKILSKENMNAAYKRVCANKGAGGVDGVTVEELGDYIKEHWRSIREQIRQRQYKPQPVRRVEIPKQNGGVRKLGIPTVM | 549 |
| | DRVIQQGIVQVINPMCEPLFSEWSYGFRPNRSCEMAIRQLLIYLNEGYEWIVDIDLEKFFDNVPQDRLMSLVHVIINDGDTESL | |
| | IRKYLKAGVMTPQGYEETRIGTPQGGNLSPLLSNIMLNELDKELEARGLRFTRYADDCVIALKSESSAKRVMRTVSDWIQRKLG | |
| | LKVNMTKTHITKPLKLKYLGFGFYKDSKTKEWKCRAHQDSIVKLKRKLKELTCRKMPGTVREKIEKINQVTRGWINYYALGSMK | |
| | TAMTAIDAHLRTRLRVIIWKQWKVPKRRQWGLQKLGVGKDLARLTSYCGDRYQWVVTKTCVVRAISKEVLTKAGLISCLEYYNK | |
| | RHALKLC | |
| 21534 | MSELLEKILSKENMNTAYKRVCANKGAGGVDGVTVEELGDYIKEHWRSIGEQIRQRQYKPQPVRRVEIPKQNGGVRKLGIPTVM | 550 |
| | DRVIQQGIVQVINPMCEPLFSEWSYGFRPNRSCEMAIRQLLIYLNEGYEWIVDIDLEKFFDNVPQDRLMSLVHVIINDGDTESL | |
| | IRKYLKAGVMTPQGYEETRLGTPQGGNLSPLLSNIMLNELDKELEARGLRFTRYADDCVIAVKSETSAKRVMRTVSDWIQRKLG | |
| | LKVNMTKTHITRPLELKYLGFGFYKDSKTKEWKCRAHQDSIVKIKRRLKELTCRKTPGKVREKIEKINQVTRGWINYYALGSMK | |
| | TAMTAIDAHLRTRLRVIIWKRWKVPKKRQWGLQKLGIGKDLARLTSYCGDRYQWVVTKTCVVRAISEEVLAKAGLISCLEYYNK | |
| | RHALKLC | |
| 21535 | MSELLDKILHKDNMNEAYKKVCANKGAGGVDGVSTEELGEYIKTNWPTIKEQILRREYKPQPVRRVEIPKPNGGKRKLGIPTAM | 551 |
| | DRVIQQAIVQVIGPICDPHFSEFSYGFRPNRSCEMAIRKVLEYLNEDYTWIVDIDLEKFFDNVPQDRLLSYVHILINDGDTESL | |
| | IRKYLKVGVMTPEGYEHTEMGTPQGGNLSPLLSNIMLNELDKELENRGLHFARYADDCIIAVKSEASAKRVMNSITSWIERKLG | |
| | LKVNAEKTKIVRPTKVKYLGFGFYKDTKTLEWKCKPHQDSVAKFKRRLKALTQRKNSMRLGIRIKKINQVTRGWINYFALGSMK | |
| | MAISDIDAHLRTRLRTIIWKQWKVPSKRQWGLQKLGVSKDLARLTSYCGDRYQWVVTKTCVKRAISKEVLGRAGLISCLDYYNE | |
| | RHALKLN | |
| 21536 | MSELLDKILDRENMNEAYKRVCANKGAGGVDGVSTEELGDYIRKNWPEIKEQIKRREYKPQPVRRVEIPKPSGGKRKLGIPTAM | 552 |
| | DRVIQQAIVQAIGPICDPHFSEFSYGFRPKRSCEMAIRKVLEYLNEDYTWIVDIDLEKFFDNVPQDRLMSYLHILINDGDTESL | |
| | ICKYLKAGVMTPEGYERTEMGTPQGGNLSPLLSNIMLNELDKELEHRGLHFARYADDCIIAVKSEASAKRVMYSITSWIERKLG | |
| | LKVNAEKTKIVRPTNLKYLGFGFYKDYKADEWKCKPHQEAIAKFKRKLKALTQRKSSMKLGLRIEKINQVTRGWINYFALGSMK | |
| | MALADIDAHLRTRLRTIIWKQWKVPSKRQWGLQKLGVNKDLARLTSYRGNRYQWVVTKTCVVRAISKEVLGKAGLISCLDYYTE | |
| | RHALKLS | |
| 21537 | MSELLEKILDKRNMNEAYKKVCANKGAGGVDGMEIDELDGYIRGNWESIKEQIRKRSYTPQPVRRVEIPKSNGSKRKLGIPTVM | 553 |
| | DRVIQQGIAQVISPMCEPLFSGNSYGFRPNRSCEMAIREMLVFLNEGYEWIVDIDLEKFFDNVPQDRLMSLVHNIINDGDTESL | |
| | IRKYLKAGVMIQGRYEKTDRGTPQGGNLSPLLSNIMLNELDKELEKRGLRFVRYADDCVIAVRSEASAKRVMYSITDWIERKLG | |
| | LKVNAEKTHITRPGKLKYLGFGFYKDPKAKEWKSRPHGESVAKFRRTLKKLTNRSQSMAFAERVQKLNRVIRGWINYFALGSMK | |
| | TAMNDIDAHLRTRLRVIIWKQWKVPSKRQWGLQKLGIGEDLARVTAYCGNRYQWIVTKTCVVRAISKEKLSQAGLVSCYDYYME | |
| | RHALKLC | |
| 21538 | MSELLDRILSRDNMNLAYKRVKANKGAGGIDDVEVDELYSYIKENWKSIEGQIRQRTYKPQPVRRVEIPKPNGGKRELGIPTVM | 554 |
| | DRVIQQAIVQVISPMCEPYFSDRSYGFRPNRSCETAIRKVLEYLNDGYTWIVDIDLEKFFDNVPQDRLMSLVHRIIDDGDTESL | |
| | IRKYLKAGVMVRGKYERTELGTPQGGNLSPLLSNIMLNELDRELEKRGLNFTRYADDCIIAVRSEASAKRVMHRITEWIERKLG | |
| | LKVNATKTKITRPSGLKYLGFGFYRDPKADEWKCKPHKDSIAKFKHKLKQLTERRWSINFRVRIAKINQVTRGWINYYALGSMK | |
| | TAMAEIDAHLRTRLRMIIWKQWKVPAKRQWGLQKLGIGKDLARVTAYCGNRYYWVVTKTCVVRAISKEKLALAGIVSLSDYYAE | |
| | RHMLKLI | |
| 21539 | MSEMLERILDDENIKTAYKRVYANKGAGGVDGVTTEELEEYMRVNWRSIKEQIRERKYNPQAVKRVEIPKPNGGIRKLGIPTVI | 555 |
| | DRVIEQAITQILTPIFDPLFSDSSYGFRPNRRCEQAIIKLLEHENDGYVWIVDIDLEKFFDNVPQDKLMSYVGRVIHDGDTESL | |
| | IRKYLKAGVMVNGRYEATNLGTPQGGNLSPLLSNIMLDELDKELENRGLHFTRYADDCVIVLKSKAAATRVMYSITDWIERKLG | |
| | LKVNATKTKITPPNKLKYLGFGFWKDKQEWKARPHEDSVEKLKRKLKALCKRNWSIDLTTRIKKINEVTRGWINYFRIASMKSK | |
| | LQNIDEHLRTQLRVIIWKQWKVPKKREWGLAKLGVGKDLARLTAYCGDRYQWVVTKTCVVRAISKEKLTKKGLVSCLDYYAERR | |
| | HILNLN | |
| 21540 | MSELLEKILDRDNMNAAYKRVCANKGAGGIDGVTVEELSDYIKENWGSIREQIRQRKYKPQPVRRVEIPKLNGGVRKLGIPTVM | 556 |
| | DRVIQQGIVQVVSPMCEPMFSERSYGFRPNRSCEMAIRQLLVYLNEGYEWIVDIDLEKFFDNVPQDRLMSLVHDIVKDGDTESL | |
| | IRKYLKAGVMTRQGYEETNLGTPQGGNLSPLLSNIMLDKLDKELEARGLRFARYADDCVIAVKSEASAKRVMHTVTDWIQRKLG | |
| | LKVNMTKTHITRPMKLKYLGFGFYKDNKTEEWKCRPHKDSIVRLKSKLKELTCRRMPGKVSEKIKRINQVTRGWINYYALGSMK | |
| | TVMAEVDAHLRTRLRMNIWKQWKVPKKRQWGLQKLGVDKDSARLTAYYGNHYYKVVKGTCVKRAISKEVLARAGLI | |
| 21541 | MLEKILSKDNMNKAYKAVAANKGSSGVDNVTIEELGSFIKEYWIEIREQIRQRQYKPQPVRRVEIPKPNGGIRKLGIPTVMDRV | 557 |
| | IQQAIVQVISPICEPYFSDRSYGFRPGRSCEMAIVKLLEYLNDGYEWIVDIDLEKFFDNVPQDKLMSYVHNIIDDGDTESLIRK | |
| | YLKAGVMTPEGYEETRLGTPQGGNLSPLLSNIMLNELDKELEARGLNFARYADDCVIAAKTDMSARRIMRNITSWIERKLGLKV | |
| | NATKTKVVRPTKLKYLGFGFYKDIQTKKWMSRPHEESVEKFKRKLKQLTCRSTSMKFKIRINKLNQVIRGWINYFSLGSMKTKM | |
| | KEIDEHLRTRLRVVLWKQWKKPSKRQWALQKLGIGSDLARQTAYMGDHYQWVVTKTCVVRAISKENLGQFGLVSCLDYYTERHS | |
| | LKFN | |
| 21542 | MLQLLEEVLDRNNMNLAYEKVYANKGAGGVDGVTLDELKAYIKENWPDVQTQIRNRTYQPKPVKRVEIPKANGGTRNLGIPTVM | 558 |
| | DRVIQQAMVQVLSPICEPHFSEYSYGFRPKRSCEMAILKLLDYENDGFLWIVDIDLEKFFDNVPQDRLMSYVHNIINDGDTESL | |
| | IRKYLKAGVMINGVKHKTEVGTPQGGNLSPLLSNIMLTELDKELEARGLKFTRYADDCIIVVKSEASAKRVMRTITDWIERKLG | |
| | LKVNMTKTQVTGPTKLKYLGFGFYFDYKSKTWKTRPHEDSVTKFKRKLKQLTKRNWSINLRERIKKINGVIRGWINYFSLCSMK | |
| | THMEKIDRHLRTRIRVIVWKQWKVPSKRQWGLQKLGIGRDLAKQTSYMGNHYQWVVTKTCVVRAISKEKLAQAGLVHCLDYYLN | |
| | QHTLKFNRTAVCRTACTVV | |
| 21543 | MSELLEKILNRENMNKAYKRVKANKGTSGIDEITIEDAYVYIKENWESIRAEITERKYKPQPVKRVEIPKPNGGTRNLGIPTVM | 559 |
| | DRIIQQAMVQVLSPICETFFSDYSYGFRPNRSCEQAINKLLEYINDGYEWIVDIDLEKFFDNVPQDKLMSYVHIIINDGDTESL | |
| | IRKYLKAGIMINGKYEKSEKGTPQGGNLSPLLSNIILNELDKELESRGLHFTRYADDCVIAVKSRASANRVMHTITKWIEHKLG | |
| | LKVNATAAAGLKVNATKTHITRPNKLKYLGFGFYYDTKAEKYCARPHASSIQRFKRKLKQLTIRKNTMALKERIRRLNQVIRGW | |
| | INYYSICNMKTHMTRIDEHLRTRLRVIIWKQWKVPSKRQWGLQKLGISKDRARQTSYMGDHYQWVVTKTCVVRAISKEKLAQKG | |
| | LVSCLDYYIERHALKLKRTAVYGTVRTVV | |
| 21544 | MSELLEKILNRDNMNQAYKRVKANKGTSGIDEVTVNEAYGYIEENWERIKQEITERRYKPQPVKRVEIPKPNGGTRNLGIPTVM | 560 |
| | DRIIQQAMVQVLSPMCEEIFSEYSYGFRPGRSCEQAIIKVLEYENDGYGWIVDIDLEKFFDNVPQDKLMSYVHTIINDGDTESL | |
| | IRKFLKAGIMINGRYEESEKGTPQGGNLSPLLSNIILSELDKELESRGLKFVRYADDCVIAVKSRASANRVMHTITSWIEGKLG | |
| | LKVNATKTQITKPNKLKYLGFGFYYHPKEKKYCARPHESSIQQFKRKLKRLTIRKNTMTLEVRIKQLNQVIRGWINYYAIGNMK | |
| | THMARIDSHLRTRLRVIIWKQWKVPSKRQWGLQKLGINKDRARQTSYMGDHYQWVVTKTCVVRAISKERLTQKGLVSCLDYYMD | |
| | RHNLKLERTAVYGTVRTVV | |
| 21551 | MRNVNEQILHYLGRIHENGKEKAHKAMSELLEKILSKDNMNAAYKRVYANKGAGGVDDVTVGELGGYIKENWESIREQIRRREY | 561 |
| | KPQPVRRVEIPKPNGGVRELGIPTVMDRVIQQGIVQVISPVCEPLFSECSYGFRPNRSCEMAVRQLLIYLNEGYEWIVDIDLER | |
| | FFDNVPQDKLMSLVHNIINDGDTESLIRKYLKAGVMTPDGYEETKLGTPQGGNLSPLLSNIMLNELDKELEARGLRFTRYADDC | |
| | VIAVRSESSAKRVMRTVSDWIQRKLGLKVNTTKTHITRPQKLKYLGFGFYKDSKAKEWKCRPHQESVKKLKRRLKELTCRKMPG | |
| | TVTGRIAKINQVTRGWINYYALGSMKTAMTEIDAHLRTRLRIIIWKQWKVPKKRQWGLQKLGVDKDLARLTAYCGDRYYWVATK | |
| | TCVVRAISKDVFAKAGLISCLDYYNERHALKLC | |
| 21560 | MSELLEKILSKENMNTAYKRVCANKGAGGVDGVTVEELGDYIKEHWRSIREQIRQRQYKPQPVRRVEIPKQNGGVRELGIPTVM | 562 |
| | DRVIQQGIVQVINPMCEPLFSEWSYGFRPNRSCEMAIRQLLIYLNEGYEWIVDIDLEKFFDNVPQDRLMSLVHDIINDGDTESL | |
| | IRKYLKAGVMTPQGYEETRIGTPQGGNLSPLLSNIMLNELDKELEARGLRFTRYADDCVIAVKSESSAKRVMRTVSDWIQRKLG | |
| | LKVNMTKTHITRPLKLKYLGFGFYKDSKTKEWKCRAHQDSIVKLKRKLKELTCRKTPGKVREKIEKINQVTRGWINYYALGSMK | |
| | TAMTAIDAHLRTRLRVIIWKQWKVPKKRQWGLQKLGVGKDLARLTSYCGDRYQWVVTKTCVVRAISKEVLAKAGLISCLEYYNK | |
| | RHALKLC | |
| 21564 | MSELLEKILSKNNMNAAYKKVCANKGAGGVDNVTVEELGDYIKENWESIREQIRRREYKPQPVRRVEIPKPDGGVRKLGIPTVM | 563 |
| | DRVIQQGIVQVISPMCEPVFSEWSYGFRPNRSCEMAVRQLLVYLNEGYEWIVDIDLERFFDNVPQDRLMSLVHDIINDGDTESL | |
| | IRKYLKAGVMSRQGYEETKLGTPQGGNLSPLLSNIMLNELDKELEARGLRFTRYADDCVIVVRSESSAKRVMRTVADWIQRKLG | |
| | LKVNMTKTHITRPQKLKYLGFGFYRDSRAKEWKCRPHQDSVKKFKRKLKELTCRKMPGTVTGRIVQINQVTRGWINYYALGSMK | |
| | TAMTEIDAHLRTRLRIIIWKQWKVPKKRQWGLQKLGIGKDLARLTAYCGDRYYWVATKTCVVRAISKDVFARSGLISCLDYYNE | |
| | RHALKLC | |
| 21566 | MSELLEKILSKKNMNAAYKKICANKGAGGVDDVTVEELGDYIKENWESIREQIRRREYKPQPVRRVEIPKPDGGVRKLGIPTVM | 564 |
| | DRVIQQGIVQIISPMCEPLFSEWSYGFRPNRSCEMAVRQLLMYLNEGYEWIVDIDLEKFFDNVPQDRLMSLVHDIINDGDTESL | |
| | IRKYLKAGVMTPQGYKETKLGTPQGGNLSPLLSNIMLNELDKELEARGLRFTRYADDCVIAVRSESSAKRVMRTVIDWIQRKLG | |
| | LKVNMTKTHITRPQKLKYLGFGFYKDSKAKEWKCRPHQDSVKKFKRKLKELTCRKMPGTVTGKIVKINQVTRGWVNYYALGSMK | |
| | TAMTEIDAHLRTRLRIIIWKQWKAPKKRQWGLQKLGIDKDLARLTAYCGDRYYWVATKTCVNRAISKKVLTKAGLVSCLDYYNE | |
| | RHALKLC | |
| 21572 | MSELLEKILSQNNMNAAYKKVCANKGAGGVDNVTVEELGDYIKENWESIREQIRRREYKPQPVRRVEIPKPDGGVRKLGIPTVM | 565 |
| | DRVIQQGIVQVISPMCEPMFSEWSYGFRPNRSCEMAIRQLLVYLNEGYEWIVDIDLERFFDNVPQDRLMSLVHDIINDGDTESL | |
| | IRKYLKAGVMSRQGYEETKLGTPQGGNLSPLLSNIMLNELDKELGARGLRFTRYADDCVIAVSSESSAKRVMRTVADWIQRKLG | |
| | LKVNMTKTHITRPQKLKYLGFGFYKDSREKQWKCRPHQDSVKKFKRKLKELTCRKMPGTVTGKIVQINQVTRGWINYYALGSMK | |
| | TAMTEIDAHLRTRLRIIIWKQWKVPKKRQWGLQKLGIGKDLARLTAYCGDRYYWVATKTCVVRAISKDVFAKAGLISCLDYYNE | |
| | HHALKLC | |
| 21573 | MSRYCNHLGQIHENGKEKAHKAMSELLEKVLSKDNMNAAYKRVCANKGAGGVDDVTVEELGDYIKENWEGIREQIRQREYRPQP | 566 |
| | VRRVEIPKPNGGVRKLGIPTVMDRVIQQGIVQIISPMCEPLFSERSYGFRPDRSCEMAVRQLLVYLNEGYEWIVDIDLEKFEDN | |
| | VPQDRLMSLVHDIIKDGDTESLIRKYLKAGVMTPQGYEETNLGTPQGGNLSPLLSNIMLNELDKELEERGLRFTRYADDCVIAV | |
| | KSEASAKRVMRTVTGWIQRKLGLKVNMTKTRITRPQRLKYLGFGLYKDSKAKEWKCRPHQESVKKFKGRLKELTCRKMPGTVTG | |
| | RIAEINQVTRGWINYYALGSMKTAMTETDAHLRTRLRIIIWKQWKVPKKRQWGLQKLGVSKDLARLTAYCGDRYYWVATKTCVV | |
| | RAISKDVFAKAGLVSCLDYYNERHALKLC | |
| 21574 | MSELLEKILSKNNMNAAYKKICANKGAGGVDDVTVEELGDYIKENWESIREQIRRREYKPQPVRRVEIPKSDGGVRKLGIPTVM | 567 |
| | DRVIQQGIVQIISPMCEPLFSEWSYGFRPNRSCEMAVRQLLMYLNEGYEWIVDIDLEKFFDNVPQDRLMSLVHDIINDGDTESL | |
| | IRKYLKAGVMTPQGYKETKFGTPQGGNLSPLLSNIMLNELDKELEARGLRFTRYADDCVIAVRSESSAKRVMRTVIDWIQRKLG | |
| | LKVNMTKTHITRPQKLKYLGFGFYKDSKAKEWKCRPHQDSVKKFKRKLKELTCRKMPGTVTGKIVKINQVTRGWVNYYALGSMK | |
| | TAMTEIDAHLRTRLRIIIWKQWKAPKKRQWGLQKLGIDKDLARLTAYCGDRYYWVATKTCVNRAISKKVLTKAGLVSCLDYYNE | |
| | RHALKLC | |

TABLE 4
Exemplary Engineered RDDPs

| | | SEQ |
|---|---|---|
| RDDP Ref | Amino Acid Sequence | ID: |
| 2691319 | MSELLEKILSKRNMNTAYKRVCANKGAGGVDDVTVEELGAYVKENWESIREQIRRREYTPQPVRRVEIPKPRG | 568 |
| (D12R-D72R- | GERKLGIPTVMDRVIQQGIVQIISPMCEPLFSKWSYGFRPNRSCEMAIRQLLEYLNEGYEWIVDIDLEKFFDN | |
| N195R) | VPQDRLMSLVHDIIKDGDTESLIRKYLKAGAMTPQGYKETKLGTPQGGRLSPLLSNIMLNELDKELEARGLRF | |
| | TRYADDCVIAVKSESSAKRVMRTVTDWIQRKLGLKVNMTKSQITRPQKLKYLGFGFHKDSKAKEWKCRPHQES | |
| | VKKFKVRLKELTCRKKPGTVTSKIAKINQVTRGWINYYALGSMKTAMTEIDAHLRTRLRVIIWKQWKVPKKRQ | |
| | WGLQKLGVSKDLARLTAYCGDSYYWVATKTCVVRAISKEVFAKSGLVSCLDYYNERHALKLC | |
| 2691319 | MSELLEKILSKRNMNTAYKRVCARKGAGGVDDVTVEELGAYVKENWESIREQIRRREYTPQPVRRVEIPKPRG | 569 |
| (D12R-N24R- | GERKLGIPTVMDRVIQQGIVQIISPMCEPLFSKWSYGFRPKRSCEMAIRQLLEYLNEGYEWIVDIDLEKFEDN | |
| D72R-N114K) | VPQDRLMSLVHDIIKDGDTESLIRKYLKAGAMTPQGYKETKLGTPQGGNLSPLLSNIMLNELDKELEARGLRF | |
| | TRYADDCVIAVKSESSAKRVMRTVTDWIQRKLGLKVNMTKSQITRPQKLKYLGFGFHKDSKAKEWKCRPHQES | |
| | VKKFKVRLKELTCRKKPGTVTSKIAKINQVTRGWINYYALGSMKTAMTEIDAHLRTRLRVIIWKQWKVPKKRQ | |
| | WGLQKLGVSKDLARLTAYCGDSYYWVATKTCVVRAISKEVFAKSGLVSCLDYYNERHALKLC | |
| 2691319 | MSELLEKILSKRNMNTAYKRVCARKGAGGVDDVTVEELGAYVKENWESIREQIRRREYTPQPVRRVEIPKPRG | 570 |
| (D12R-N24R- | GERKLGIPTVMDRVIQQGIVQIISPMCEPLFSKWSYGFRPNRSCEMAIRQLLEYLNEGYEWIVDIDLEKFFDN | |
| D72R-N195R) | VPQDRLMSLVHDIIKDGDTESLIRKYLKAGAMTPQGYKETKLGTPQGGRLSPLLSNIMLNELDKELEARGLRF | |
| | TRYADDCVIAVKSESSAKRVMRTVTDWIQRKLGLKVNMTKSQITRPQKLKYLGFGFHKDSKAKEWKCRPHQES | |
| | VKKFKVRLKELTCRKKPGTVTSKIAKINQVTRGWINYYALGSMKTAMTEIDAHLRTRLRVIIWKQWKVPKKRQ | |
| | WGLQKLGVSKDLARLTAYCGDSYYWVATKTCVVRAISKEVFAKSGLVSCLDYYNERHALKLC | |
| 2691319 | MSELLEKILSKRNMNTAYKRVCANKGAGGVDDVTVEELGAYVKENWESIREQIRRREYTPQPVRRVEIPKPRG | 571 |
| (D12R-D72R- | GERKLGIPTVMDRVIQQGIVQIISPMCEPLFSKWSYGFRPKRSCEMAIRQLLEYLNEGYEWIVDIDLEKFFDN | |
| N114K-N195R) | VPQDRLMSLVHDIIKDGDTESLIRKYLKAGAMTPQGYKETKLGTPQGGRLSPLLSNIMLNELDKELEARGLRF | |
| | TRYADDCVIAVKSESSAKRVMRTVTDWIQRKLGLKVNMTKSQITRPQKLKYLGFGFHKDSKAKEWKCRPHQES | |
| | VKKFKVRLKELTCRKKPGTVTSKIAKINQVTRGWINYYALGSMKTAMTEIDAHLRTRLRVIIWKQWKVPKKRQ | |
| | WGLQKLGVSKDLARLTAYCGDSYYWVATKTCVVRAISKEVFAKSGLVSCLDYYNERHALKLC | |
| 2691319 | MSELLEKILSKRNMNTAYKRVCARKGAGGVDDVTVEELGAYVKENWESIREQIRRREYTPQPVRRVEIPKPRG | 572 |
| (D12R-N24R- | GERKLGIPTVMDRVIQQGIVQIISPMCEPLFSKWSYGFRPKRSCEMAIRQLLEYLNEGYEWIVDIDLEKFFDN | |
| D72R-N114K- | VPQDRLMSLVHDIIKDGDTESLIRKYLKAGAMTPQGYKETKLGTPQGGRLSPLLSNIMLNELDKELEARGLRF | |
| N195R) | TRYADDCVIAVKSESSAKRVMRTVTDWIQRKLGLKVNMTKSQITRPQKLKYLGFGFHKDSKAKEWKCRPHQES | |
| | VKKFKVRLKELTCRKKPGTVTSKIAKINQVTRGWINYYALGSMKTAMTEIDAHLRTRLRVIIWKQWKVPKKRQ | |
| | WGLQKLGVSKDLARLTAYCGDSYYWVATKTCVVRAISKEVFAKSGLVSCLDYYNERHALKLC | |
| 2691323 | MSELLERILSNRNMNAAYKRVCANKGAGGVDGVTVEELGDYIKENWSSIREQIRRRQYKPQPVRRVEIPKPRG | 573 |
| (D12R-N72R- | GVRNLGIPTVMDRVIQQGIVQVLSPMCEPLFSERSYGFRPNRSCEMAIRQLLVYLNEGCEWIVDIDLEKFFDN | |
| N195R) | VPQDRLMSLVHDIINDGDTESLIRKYLKAGVMTPQGYEETKLGTPQGGRLSPLLSNIMLNELDKELEKRGLRF | |
| | TRYADDCVIAVKSESSAKRVMRTVADWIQRKLGLKVNMTKTHITRPTKLKYLGFGFYRDNKAKEWKCRPHQDS | |
| | VAKFKRKLKELTCRKTPGTVTGRIAKINQVTRGWINYYAIGSMKTTMIEIDAHLRTRLRVIIWKQWKVPKKRQ | |
| | WGLQKLGIGKDLARLTSYYGDRYQWIVTKTCVARAISKEVLAKAGLVSCLDYYNERHALKLC | |
| 2691323 | MSELLERILSNRNMNAAYKRVCARKGAGGVDGVTVEELGDYIKENWSSIREQIRRRQYKPQPVRRVEIPKPRG | 574 |
| (D12R-N24R- | GVRNLGIPTVMDRVIQQGIVQVLSPMCEPLFSERSYGFRPKRSCEMAIRQLLVYLNEGCEWIVDIDLEKFFDN | |
| N72R-N114K) | VPQDRLMSLVHDIINDGDTESLIRKYLKAGVMTPQGYEETKLGTPQGGNLSPLLSNIMLNELDKELEKRGLRF | |
| | TRYADDCVIAVKSESSAKRVMRTVADWIQRKLGLKVNMTKTHITRPTKLKYLGFGFYRDNKAKEWKCRPHQDS | |
| | VAKFKRKLKELTCRKTPGTVTGRIAKINQVTRGWINYYAIGSMKTTMIEIDAHLRTRLRVIIWKQWKVPKKRQ | |
| | WGLQKLGIGKDLARLTSYYGDRYQWIVTKTCVARAISKEVLAKAGLVSCLDYYNERHALKLC | |
| 2691323 | MSELLERILSNRNMNAAYKRVCARKGAGGVDGVTVEELGDYIKENWSSIREQIRRRQYKPQPVRRVEIPKPRG | 575 |
| (D12R-N24R- | GVRNLGIPTVMDRVIQQGIVQVLSPMCEPLFSERSYGFRPNRSCEMAIRQLLVYLNEGCEWIVDIDLEKFFDN | |
| N72R-N195R) | VPQDRLMSLVHDIINDGDTESLIRKYLKAGVMTPQGYEETKLGTPQGGRLSPLLSNIMLNELDKELEKRGLRF | |
| | TRYADDCVIAVKSESSAKRVMRTVADWIQRKLGLKVNMTKTHITRPTKLKYLGFGFYRDNKAKEWKCRPHQDS | |
| | VAKFKRKLKELTCRKTPGTVTGRIAKINQVTRGWINYYAIGSMKTTMIEIDAHLRTRLRVIIWKQWKVPKKRQ | |
| | WGLQKLGIGKDLARLTSYYGDRYQWIVTKTCVARAISKEVLAKAGLVSCLDYYNERHALKLC | |
| 2691323 | MSELLERILSNRNMNAAYKRVCANKGAGGVDGVTVEELGDYIKENWSSIREQIRRRQYKPQPVRRVEIPKPRG | 576 |
| (D12R-N72R- | GVRNLGIPTVMDRVIQQGIVQVLSPMCEPLFSERSYGFRPKRSCEMAIRQLLVYLNEGCEWIVDIDLEKFFDN | |
| N114K-N195R) | VPQDRLMSLVHDIINDGDTESLIRKYLKAGVMTPQGYEETKLGTPQGGRLSPLLSNIMLNELDKELEKRGLRF | |
| | TRYADDCVIAVKSESSAKRVMRTVADWIQRKLGLKVNMTKTHITRPTKLKYLGFGFYRDNKAKEWKCRPHQDS | |
| | VAKFKRKLKELTCRKTPGTVTGRIAKINQVTRGWINYYAIGSMKTTMIEIDAHLRTRLRVIIWKQWKVPKKRQ | |
| | WGLQKLGIGKDLARLTSYYGDRYQWIVTKTCVARAISKEVLAKAGLVSCLDYYNERHALKLC | |
| 2691323 | MSELLERILSNRNMNAAYKRVCARKGAGGVDGVTVEELGDYIKENWSSIREQIRRRQYKPQPVRRVEIPKPRG | 577 |
| (D12R-N24R- | GVRNLGIPTVMDRVIQQGIVQVLSPMCEPLFSERSYGFRPKRSCEMAIRQLLVYLNEGCEWIVDIDLEKFFDN | |
| N72R-N114K- | VPQDRLMSLVHDIINDGDTESLIRKYLKAGVMTPQGYEETKLGTPQGGRLSPLLSNIMLNELDKELEKRGLRF | |
| N195R) | TRYADDCVIAVKSESSAKRVMRTVADWIQRKLGLKVNMTKTHITRPTKLKYLGFGFYRDNKAKEWKCRPHQDS | |
| | VAKFKRKLKELTCRKTPGTVTGRIAKINQVTRGWINYYAIGSMKTTMIEIDAHLRTRLRVIIWKQWKVPKKRQ | |
| | WGLQKLGIGKDLARLTSYYGDRYQWIVTKTCVARAISKEVLAKAGLVSCLDYYNERHALKLC | |

TABLE 5
Exemplary Target Gene
Exemplary Target Gene

AAVS1, ABCA4, ABCB11, ABCC8, ABCD1, ABCG5, ABCG8, ACAD9, ACADM, ACADVL, ACAT1, ACOX1, ACSF3, ADA,
ADAMTS2, ADGRG1, AGA, AGL, AGPS, AGXT, AHI1, AIRE, ALDH3A2, ALDOB, ALG6, ALK, ALKBH5, ALMS1, ALPL, AMRC9,
AMT, ANAPC10, ANAPC11, ANGPTL3, ANGPLT7, APC, Apo(a), APOCIII, APOE, APOEε4, APOL1, APP, AQP2, AR, ARFRP1,
ARG1, ARH, ARL13B, ARL6, ARSA, ARSB, ASL, ASNS, ASPA, ASS1, ATM, ATP6V1B1, ATP7A, ATP7B, ATRX, ATXN1, ATXN10,
ATXN2, ATXN3, ATXN7, ATXN8OS, AXIN1, AXIN2, B2M, BACE-1, BAK1, BAP1, BARD1, BAX2, BBS1, BBS10, BBS12, BBS2,
BCKDHA, BCKDHB, BCL2L2, BCS1L, BEST1, Betaglobin gene, BLM, BMPR1A, BRAF, BRAFV600E, BRCA1, BRCA2, BRIP1,
BSND, C9orf72, CA4, CACNA1A, CAPN3, CASR, CBS, CCNB1 CC2D2A, CCR5, CD1, CD2, CD3, CD3D, CD3Z, CD4, CD5,
CD6, CD7, CD8A, CD8B, CD9, CD14, CD18, CD19, CD21, CD22, CD23, CD27, CD28, CD30, CD33, CD34, CD36, CD38,
CD40, CD40L, CD44, CD46, CD47, CD48, CD52, CD55, CD57, CD58, CD59, CD68, CD69, CD72, CD73, CD74, CD79A, CD80,
CD81, CD83, CD84, CD86, CD90, CD93, CD96, CD99, CD100, CD123, CD160, CD163, CD164, CD164L2, CD166, CD200,
CD204, CD207, CD209, CD226, CD244, CD247, CD274, CD276, CD300, CD320, CDC73, CDH1, CDH23, CDK11, CDK4,
CDKN1A, CDKN1B, CDKN1C, CDKN2A, CDKN2B, CEBPA, CELA3B, CEP290, CERKL, CFB, CFTR, CHCHD10, CHEK2,
CHM, CHRNE, CIITA, CLN3, CLN5, CLN6, CLN8, CLRN1, CLTA, CNBP, CNGB1, CNGB3, COL1A1, COL1A2, COL27A1,
COL4A3, COL4A4, COL4A5, COL7A1, CPS1, CPT1A, CPT2, CRB1, CREBBP, CRX, CRYAA, CTNNA1, CTNNB1, CTNND2,
CTNS, CTSK, CXCL12, CYBA, CYBB, CYP11B1, CYP11B2, CYP17A1, CYP19A1, CYP27A1, DBT, DCC, DCLRE1C, DERL2,
DFNA36, DFNB31, DGAT2, DHCR7, DHDDS, DICER1, DIS3L2, DLD, DMD, DMPK, DNAH5, DNAI1, DNAI2, DNM2, DNMT1,
DPC4, DUX4, DYSF, EDA, EDN3, EDNRB, EGFR, EIF2B5, EMC2, EMC3, EMD, EMX1, EN1, EPCAM, ERCC6, ERCC8, ESCO2,
ETFA, ETFDH, ETHE1, EVC, EVC2, EYS, F5, F9, FXI, FAH, FAM161A, FANCA, FANCB, FANCC, FANCD1, FANCD2, FANCE,
FANCF, FANCG, FANCI, FANCJ, FANCL, FANCM, FANCN, FANCP, FANCS, FBN1, FGF14, FGFR2, FGFR3, FGA, FGB,
FGG, FH, FHL1, FIX, FKRP, FKTN, FLCN, FMR1, FOXP3, FSCN2, FUS, FUT8, FVIII, FXII, FXN, G6PC, GAA, GALC, GALK1,
GALT, GAMT, GATA2, GATA-4, GBA, GBE1, GCDH, GCGR, GDNF, GFAP, GFM1, GHR, GJB1, GJB2, GLA, GLB1, GLDC,
GLE1, GNE, GNPTAB, GNPTG, GNS, GPC3, GPR98, GREM1, GRHPR, GRIN2B, H2AFX, H2AX, HADHA, HAX1, HBA1, HBA2,
HBB, HER2, HEXA, HEXB, HFE, HGSNAT, HLCS, HMGCL, HOGA1, HOXB13, HPRPF3, HPRT1, HPS1, HPS3, HRAS, HRD1,
HSD17B4, HSD3B2, HTT, HUS1, HYAL1, HYLS1, IDS, IDUA, IFITM5, IKBKAP, IL2RG, IL7R, IMPDH1, INPP5E, IRF4, ITGB2,
ITPR1, IVD, JAG1, JAK1, JAK3, KCNC3, KCND3, KCNJ11, KLHL7, KRAS, LAMA2, LAMA3, LAMB3, LAMC2, LCA5, LDLR,
LDLRAP1, LHX3, LIFR, LIPA, LMNA, LOR, LOXHD1, LPL, LRAT, LRP6, LRPPRC, LRRK2, MADR2, MAN2B1, MAPT, MAX,
MCM6, MCOLN1, MECP2, MED17, MEFV, MEN1, MERTK, MESP2, MET, METex14, MFN2, MFSD8, MIA3, MITF, MKL2,
MKS1, MLC1, MLH1, MLH3, MMAA, MMAB, MMACHC, MMADHC, MMD, MPI, MPL, MPV17, MSH2, MSH3, MSH6,
MTHFD1L, MTHFR, MTM1, MTRR, MTTP, MUT, MUTYH, MYC, MYH7, MYOC, MYO7A, NAGLU, NAGS, NAV1.7, NBN,
NDRG1, NDUFAF5, NDUFS6, NEB, NF1, NF2, NKX2-5, NOG, NOTCH1, NOTCH2, NPC1, NPC2, NPHP1, NPHS1, NPHS2,
NRAS, NR2E3, NTHL1, NTRK, NTRK1, OAT, OCT4, OFD1, OPA3, OTC, PAH, PALB2, PAQR8, PAX3, PC, PCCA, PCCB,
PCDH15, PCSK9, PD1, PDCD1, PDE6B, PDGFRA, PDHA1, PDHB, PEX1, PEX10, PEX12, PEX13, PEX14, PEX16, PEX19,
PEX2, PEX26, PEX3, PEX5, PEX6, PEX7, PFKM, PHGDH, PHOX2B, PKD1, PKD2, PKHD1, PKK, PLEKHG4, PMM2, PMP22,
PMS1, PMS2, PNPLA3, POLD1, POLE, POMGNT1, POT1, POU5F1, PPM1A, PPP2R2B, PPT1, PRCD, PRKAG2, PRKAR1A,
PRKCG, PRNP, PROM1, PROP1, PRPF31, PRPF8, PRPH2, PRPS1, PSAP, PSD95, PSEN1, PSEN2, PSRC1, PTCH1, PTEN,
PTS, PUS1, PYGM, RAB23, RAD50, RAD51C, RAD51D, RAG1, RAG2, RAPSN, RARS2, RB1, RDH12, RECQL4, RET, RHO,
RICTOR, RMRP, ROS1, RP1, RP2, RPE65, RPGR, RPGRIP1L, RPL32P3, RPTOR, RS1, RTCA, RTEL1, RUNX1, SACS, SAMHD1,
SCN1A, SCN2A, SDHA, SDHAF2, SDHB, SDHC, SDHD, SEL1L, SEPSECS, SERPINA1, SERPING1, SGCA, SGCB, SGCG, SGSH,
SIRT1, SLC12A3, SLC12A6, SLC17A5, SLC22A5, SLC25A13, SLC25A15, SLC26A2, SLC26A4, SLC35A3, SLC35B4 SLC37A4,
SLC39A4, SLC4A11, SLC6A8, SLC7A7, SMAD3, SMAD4, SMARCA4, SMARCAL1, SMARCB1, SMARCE1, SMN1, SMPD1,
SNAI2, SNCA, SNRNP200, SOD1, SOX10, SPARA7, SPTBN2, STAR, STAT3, STK11, SUFU, SUMF1, SYNE1, SYNE2, SYS1,
TARDBP, TAT, TBK1, TBP, TCF4, TCIRG1, TCTN3, TECPR2, TERC, TERT, TFR2, TGFBR2, TGM1, TH, TLE3, TMEM127,
TMEM138, TMEM216, TMEM43, TMEM67, TMPRSS6, TOP1, TOPORS, TP53, TPP1, TRAC, TRMU, TSC1, TSC2, TSFM,
TSPAN14, TTBK2, TTC8, TTPA, TTR, TULP1, TYMP, UBE2G2, UBE2J1, UBE3A, USH1C, USH1G, USH2A, VEGF, VHL,
VPS13A, VPS13B, VPS35, VPS45, VRK1, VSX2, VWF, WAS, WDR19, WDR48, WNT10A, WRN, WS2B, WS2C, WT1, XPA, XPC,
XPF, XRCC3, YAP1, ZAC1, ZEB1, ZFYVE26, and ZNF423

TABLE 6
Diseases and Syndromes
Exemplary Diseases and Syndromes

11-hydroxylase deficiency; 17,20-desmolase deficiency; 17-hydroxylase deficiency; 3-hydroxyisobutyrate aciduria; 3-
hydroxysteroid dehydrogenase deficiency; 46,XY gonadal dysgenesis; AAA syndrome; ABCA3 deficiency; ABCC8-associated
hyperinsulinism; aceruloplasminemia; acromegaly; achondrogenesis type 2; acral peeling skin syndrome; acrodermatitis
enteropathica; adrenocortical micronodular hyperplasia; adrenoleukodystrophies; adrenomyeloneuropathies; Aicardi-Goutieres
syndrome; Alagille disease (also called Alagille Syndrome); Alexander Disease, Alpers syndrome; alpha-1 antitrypsin deficiency
(AATD); alpha-mannosidosis; Alstrom syndrome; Alzheimer's disease; amebic dysentery; amelogenesis imperfecta; amish type
microcephaly; amyotrophic lateral sclerosis (ALS); anaplastic large cell lymphoma; anauxetic dysplasia; androgen insensitivity
syndrome; angiopathic thrombosis; antiphospholipid syndrome; Antley-Bixler syndrome; APECED, Apert syndrome, aplasia of
lacrimal and salivary glands, argininemia, arrhythmogenic right ventricular dysplasia, Arts syndrome, ARVD2, arylsulfatase
deficiency type metachromatic leokodystrophy, ataxia telangiectasia, autoimmune lymphoproliferative syndrome; autoimmune
polyglandular syndrome type 1; autosomal dominant anhidrotic ectodermal dysplasia; autosomal dominant deafness; autosomal
dominant polycystic kidney disease; autosomal recessive microtia; autosomal recessive renal glucosuria; autosomal visceral
heterotaxy; babesiosis; balantidial dysentery; Bardet-Biedl syndrome; Bartter syndrome; basal cell nevus syndrome; Batten disease;
benign recurrent intrahepatic cholestasis; beta-mannosidosis; β-thalassemia; Bethlem myopathy; Blackfan-Diamond anemia;
bleeding disorder (coagulation); blepharophimosis; Byler disease; C syndrome; CADASIL; calcific aortic stenosis; calcification of
joints and arteries; carbamyl phosphate synthetase deficiency; cardiofaciocutaneous syndrome; Carney triad; carnitine
palmitoyltransferase deficiencies; cartilage-hair hypoplasia; cblC type of combined methylmalonic aciduria; CD18 deficiency;
CD3Z-associated primary T-cell immunodeficiency; CD40L deficiency; CDAGS syndrome; CDG1A; CDG1B; CDG1M; CDG2C;
CEDNIK syndrome; central core disease; centronuclear myopathy; cerebral capillary malformation; cerebrooculofacioskeletal
syndrome type 4; cerebrooculogacioskeletal syndrome; cerebrotendinous xanthomatosis; Chaga's Disease; Charcot Marie Tooth
Disesase; cherubism; CHILD syndrome; chronic granulomatous disease; chronic recurrent multifocal osteomyelitis; citrin
deficiency; classic hemochromatosis; CNPPB syndrome; cobalamin C disease; Cockayne syndrome; coenzyme Q10 deficiency;
Coffin-Lowry syndrome; Cohen syndrome; combined deficiency of coagulation factors V; common variable immune deficiency 3;
complement hyperactivation; complete androgen insentivity; cone rod dystrophies; conformational diseases; congenital bile adid
synthesis defect type 1; congenital bile adid synthesis defect type 2; congenital defect in bile acid synthesis type; congenital
erythropoietic porphyria; congenital generalized osteosclerosis; Cornelia de Lange syndrome; coronary heart disease; Cousin
syndrome; Cowden disease; COX deficiency; Cri du chat syndrome; Crigler-Najjar disease; Crigler-Najjar syndrome type 1;
Crisponi syndrome; Crouzon syndrome; Currarino syndrome; Curth-Macklin type ichthyosis hystrix; cutis laxa; cystic fibrosis;
cystinosis; d-2-hydroxyglutaric aciduria; DDP syndrome; Dejerine-Sottas disease; dementia; Denys-Drash syndrome; Dercum
disease; desmin cardiomyopathy; desmin myopathy; DGUOK-associated mitochondrial DNA depletion; diabetes Type I; diabetes
Type II; disorders of glutamate metabolism; distal spinal muscular atrophy type 5; DNA repair diseases; dominant optic atrophy;
Doyne honeycomb retinal dystrophy; Dravet Syndrome; Duchenne muscular dystrophy; dyskeratosis congenita; Ehlers-Danlos
syndrome type 4; Ehlers-Danlos syndromes; Elejalde disease; Ellis-van Creveld disease; Emery-Dreifuss muscular dystrophies;
encephalomyopathic mtDNA depletion syndrome; encephalitis; enzymatic diseases; EPCAM-associated congenital tufting
enteropathy; epidermolysis bullosa with pyloric atresia; epilepsy; facioscapulohumeral muscular dystrophy; Factor V Leiden
thrombophilia; Faisalabad histiocytosis; familial atypical mycobacteriosis; familial capillary malformation-arteriovenous; Familial
Creutzfeld-Jakob disease; familial esophageal achalasia; familial glomuvenous malformation; familial hemophagocytic
lymphohistiocytosis; familial mediterranean fever; familial megacalyces; familial schwannomatosis; familial spina bifida; familial
splenic asplenia/hypoplasia; familial thrombotic thrombocytopeniarpura; Fanconi disease (Fanconi anemia);
Facioscapulohumeral muscular dystrophy (FSHD); Feingold syndrome; FENIB; fibrodysplasia ossificans progressiva; FKTN;
Fragile X syndrome; Francois-Neetens fleck corneal dystrophy; Frasier syndrome; Friedreich's ataxia; FTDP-17; Fuchs corneal
dystrophy; fucosidosis; G6PD deficiency; galactosialidosis; Galloway syndrome; Gardner syndrome; Gaucher disease; Gitelman
syndrome; GLUT1 deficiency; GM2- Gangliosidoses (e.g., Tay Sachs Disease, Sandhoff Disease) glycogen storage disease type
1b; glycogen storage disease type 2; glycogen storage disease type 3; glycogen storage disease type 4; glycogen storage disease type
9a; glycogen storage diseases; GM1-gangliosidosis; Greenberg syndrome; Greig cephalopolysyndactyly syndrome; hair genetic
diseases; hairy cell leukemia; HANAC syndrome; harlequin type ichtyosis congenita; HDR syndrome; hearing loss;
hemochromatosis type 3; hemochromatosis type 4; hemolytic anemia; hemolytic uremic syndrome; hemophilia A; hemophilia B;
hereditary angioedema type 3; hereditary angioedemas; hereditary hemorrhagic telangiectasia; hereditary hypofibrinogenemia;
hereditary intraosseous vascular malformation; hereditary leiomyomatosis and renal cell cancer; hereditary neuralgic amyotrophy;
hereditary sensory and autonomic neuropathy type; Hermansky-Pudlak disease; HHH syndrome; HHT2; hidrotic ectodermal
dysplasia type 1; hidrotic ectodermal dysplasias; histiocytic sarcoma; HNF4A-associated hyperinsulinism; HNPCC; homozygous
familial hypercholesterolemia; human immunodeficiency with microcephaly; human papilloma virus (HPV) infection; Huntington's
disease; hyper-IgD syndrome; hyperinsulinism-hyperammonemia syndrome; hypercholesterolemia; hypertrophy of the retinal
pigment epithelium; hypochondrogenesis; hypohidrotic ectodermal dysplasia; ICF syndrome; idiopathic congenital intestinal
pseudo-obstruction; immunodeficiency 13; immunodeficiency 17; immunodeficiency 25; immunodeficiency with hyper-IgM type
1; immunodeficiency with hyper-IgM type 3; immunodeficiency with hyper-IgM type 4; immunodeficiency with hyper-IgM type
5; immunoglobulin alpha deficiency; inborn errors of thyroid metabolism; infantile myofibromatosis; infantile visceral myopathy;
infantile X-linked spinal muscular atrophy; intrahepatic cholestasis of pregnancy; IPEX syndrome; IRAK4 deficiency; isolated
congenital asplenia; Jeune syndrome; Johanson-Blizzard syndrome; Joubert syndrome; JP-HHT syndrome; juvenile
hemochromatosis; juvenile hyalin fibromatosis; juvenile nephronophthisis; Kabuki mask syndrome; Kallmann syndromes;
Kartagener syndrome; KCNJ11-associated hyperinsulinism; Kearns-Sayre syndrome; Kostmann disease; Kozlowski type of
spondylometaphyseal dysplasia; Krabbe disease; LADD syndrome; late infantile-onset neuronal ceroid lipofuscinosis; LCK
deficiency; LDHCP syndrome; Leber Congenital Amaurosis Teyp 10; Legius syndrome; Leigh syndrome; lethal congenital
contracture syndrome 2; lethal congenital contracture syndromes; lethal contractural syndrome type 3; lethal neonatal CPT
deficiency type 2; lethal osteosclerotic bone dysplasia; leukocyte adhesion deficiency; Li Fraumeni syndrome; LIG4 syndrome;
lipodystrophy; lissencephaly type 1; lissencephaly type 3; Loeys-Dietz syndrome; low phospholipid-associated cholelithiasis; Lynch
Syndrome; lysinuric protein intolerance; a lysosomal storage disease (e.g., Hunter syndrome, Hurler syndrome); macular dystrophy;
Maffucci syndrome; Majeed syndrome; mannose-binding protein deficiency; mantle cell lymphoma; Marfan disease; Marshall
syndrome; MASA syndrome; mastocytosis; MCAD deficiency; McCune-Albright syndrome; MCKD2; Meckel syndrome; MECP2
Duplication Syndrome; Meesmann corneal dystrophy; megacystis-microcolon-intestinal hypoperistalsis; megaloblastic anemia type
1; MEHMO; MELAS; Melnick-Needles syndrome; MEN2s; meningitis; Menkes disease; metachromatic leukodystrophies;
methymalonic acidemia due to transcobalamin receptor defect; methylmalonic acidurias; methylvalonic aciduria; microcoria-
congenital nephrosis syndrome; microvillous atrophy; migraine; mitochondrial neurogastrointestinal encephalomyopathy;
monilethrix; monosomy X; mosaic trisomy 9 syndrome; Mowat-Wilson syndrome; mucolipidosis type 2; mucolipidosis type Ma;
mucolipidosis type IV; mucopolysaccharidoses; mucopolysaccharidosis type 3A; mucopolysaccharidosis type 3C;
mucopolysaccharidosis type 4B; multiminicore disease; multiple acyl-CoA dehydrogenation deficiency; multiple cutaneous and
mucosal venous malformations; multiple endocrine neoplasia type 1; multiple sulfatase deficiency; mycosis fungoides; myotonic
dystrophy; NAIC; nail-patella syndrome; nemaline myopathies; neonatal diabetes mellitus; neonatal surfactant deficiency;
nephronophtisis; Netherton disease; neurofibromatoses; neurofibromatosis type 1; Niemann-Pick disease type A; Niemann-Pick
disease type B; Niemann-Pick disease type C; NKX2E; non-alcoholic fatty liver disease (NAFLD); non-alcoholic steatohepatitis
(NASH); Noonan syndrome; North American Indian childhood cirrhosis; NROB1 duplication-associated DSD; ocular genetic
diseases; oculo-auricular syndrome; OLEDAID; oligomeganephronia; oligomeganephronic renal hypolasia; Ollier disease; Opitz-
Kaveggia syndrome; orofaciodigital syndrome type 1; orofaciodigital syndrome type 2; osseous Paget disease; osteogenesis
imperfecta; otopalatodigital syndrome type 2; OXPHOS diseases; palmoplantar hyperkeratosis; panlobar nephroblastomatosis;
Parkes-Weber syndrome; Parkinson's disease; partial deletion of 21q22.2-q22.3; Pearson syndrome; Pelizaeus-Merzbacher disease;
Pendred syndrome; pentalogy of Cantrell; peroxisomal acyl-CoA-oxidase deficiency; Peutz-Jeghers syndrome; Pfeiffer syndrome;
Pierson syndrome; pigmented nodular adrenocortical disease; pipecolic acidemia; Pick's disease; Pitt-Hopkins syndrome;
plasmalogens deficiency; platelet glycoprotein IV deficiency; pleuropulmonary blastoma and cystic nephroma; polycystic kidney
disease; polycystic ovarian disease; polycystic lipomembranous osteodysplasia; Pompe disease, including infantile onset Pompe
disease (IOPD) and late onset Pompe disease (LOPD); porphyrias; PRKAG2 cardiac syndrome, premature ovarian failure; primary
erythermalgia; primary hemochromatoses; primary hyperoxaluria; progressive familial intrahepatic cholestasis; propionic acidemia;
protein-losing enteropathy; pyruvate decarboxylase deficiency; RAPADILINO syndrome; renal cystinosis; retinitis pigmentosa;
Rett Syndrome; rhabdoid tumor predisposition syndrome; Rieger syndrome; ring chromosome 4; Roberts syndrome; Robinow-
Sorauf syndrome; Rothmund-Thomson syndrome; severe combined immunodeficiency disorder (SCID); Saethre-Chotzen
syndrome; Sandhoff disease; SC phocomelia syndrome; SCAS; Schinzel phocomelia syndrome; short rib-polydactyly syndrome
type 1; short rib-polydactyly syndrome type 4; short-rib polydactyly syndrome type 2; short-rib polydactyly syndrome type 3;
Shwachman disease; Shwachman-Diamond disease; sickle cell anemia; Silver-Russell syndrome; Simpson-Golabi-Behmel
syndrome; Smith-Lemli-Opitz syndrome; SPG7-associated hereditary spastic paraplegia; spherocytosis; spinocerebellar ataxia;
split-hand/foot malformation with long bone deficiencies; spondylocostal dysostosis; sporadic visceral myopathy with inclusion
bodies; storage diseases; Stargardt macular dystrophy; STRA6-associated syndrome; stroke; Tay-Sachs disease; thanatophoric
dysplasia; thyroid metabolism diseases; Tourette syndrome; transthyretin-associated amyloidosis; trisomy 13; trisomy 22; trisomy
2p syndrome; tuberous sclerosis; tufting enteropathy; urea cycle diseases; Usher Syndrome; Van Den Ende-Gupta syndrome; Van
der Woude syndrome; variegated mosaic aneuploidy syndrome; VLCAD deficiency; von Hippel-Lindau disease; von Willebrand
disease; Waardenburg syndrome; WAGR syndrome; Walker-Warburg syndrome; Werner syndrome; Wilson disease; Wiskott-
Aldrich Syndrome; Wolcott-Rallison syndrome; Wolfram syndrome; X-linked agammaglobulinemia; X-linked chronic idiopathic
intestinal pseudo-obstruction; X-linked cleft palate with ankyloglossia; X-linked dominant chondrodysplasia punctata; X-linked
ectodermal dysplasia; X-linked Emery-Dreifuss muscular dystrophy; X-linked lissencephaly; X-linked lymphoproliferative disease;
X-linked visceral heterotaxy; xanthinuria type 1; xanthinuria type 2; xeroderma pigmentosum; XPV; and Zellweger disease.

TABLE 7
Exemplary MMLV sequences

| | | | SEQ |
|---|---|---|---|
| Protein | Protein | | ID |
| Name | Type | Sequence | NO: |
| MMLV | commercial | TLNIEDEYRLHETSKEPDVSLGSTWLSDFPQAWAETGGMGLAVRQAPLIIPLKATSTPVSI | 531 |
| | RDDP | KQYPMSQEARLGIKPHIQRLLDQGILVPCQSPWNTPLLPVKKPGTNDYRPVQDLREVNK | |
| | | RVEDIHPTVPNPYNLLSGLPPSHQWYTVLDLKDAFFCLRLHPTSQPLFAFEWRDPEMGIS | |
| | | GQLTWTRLPQGFKNSPTLFDEALHRDLADFRIQHPDLILLQYVDDLLLAATSELDCQQGT | |
| | | RALLQTLGNLGYRASAKKAQICQKQVKYLGYLLKEGQRWLTEARKETVMGQPTPKTPR | |
| | | QLREFLGTAGFCRLWIPGFAEMAAPLYPLTKTGTLFNWGPDQQKAYQEIKQALLTAPAL | |
| | | GLPDLTKPFELFVDEKQGYAKGVLTQKLGPWRRPVAYLSKKLDPVAAGWPPCLRMVAA | |
| | | IAVLTKDAGKLTMGQPLVILAPHAVEALVKQPPDRWLSNARMTHYQALLLDTDRVQFG | |
| | | PVVALNPATLLPLPEEGLQHNCLDILAEAHGTRPDLTDQPLPDADHTWYTDGSSLLQEG | |
| | | QRKAGAAVTTETEVIWAKALPAGTSAQRAELIALTQALKMAEGKKLNVYTDSRYAFAT | |
| | | AHIHGEIYRRRGLLTSEGKEIKNKDEILALLKALFLPKRLSIIHCPGHQKGHSAEARGNRM | |
| | | ADQAARKAAITETPDTSTLLIENSSP | |
| MMLV | engineered | TLNIEDEYRLHETSKEPDVSLGSTWLSDFPQAWAETGGMGLAVRQAPLIIPLKATSTPVSI | 532 |
| | RDDP | KQYPMSQEARLGIKPHIQRLLDQGILVPCQSPWNTPLLPVKKPGTNDYRPVQDLREVNK | |
| | | RVEDIHPTVPNPYNLLSGLPPSHQWYTVLDLKDAFFCLRLHPTSQPLFAFEWRDPEMGIS | |
| | | GQLTWTRLPQGFKNSPTLFNEALHRDLADFRIQHPDLILLQYVDDLLLAATSELDCQQGT | |
| | | RALLQTLGNLGYRASAKKAQICQKQVKYLGYLLKEGQRWLTEARKETVMGQPTPKTPR | |
| | | QLREFLGKAGFCRLFIPGFAEMAAPLYPLTKPGTLFNWGPDQQKAYQEIKQALLTAPALG | |
| | | LPDLTKPFELFVDEKQGYAKGVLTQKLGPWRRPVAYLSKKLDPVAAGWPPCLRMVAAI | |
| | | AVLTKDAGKLTMGQPLVILAPHAVEALVKQPPDRWLSNARMTHYQALLLDTDRVQFGP | |
| | | VVALNPATLLPLPEEGLQHNCLDILAEAHGTRPDLTDQPLPDADHTWYTDGSSLLQEGQ | |
| | | RKAGAAVTTETEVIWAKALPAGTSAQRAELIALTQALKMAEGKKLNVYTDSRYAFATA | |
| | | HIHGEIYRRRGWLTSEGKEIKNKDEILALLKALFLPKRLSIIHCPGHQKGHSAEARGNRMA | |
| | | DQAARKAAITETPDTSTLLIENSSP | |

### EXAMPLES

The following examples are included for illustrative purposes only and are not intended to limit the scope of the disclosure.

### Example 1

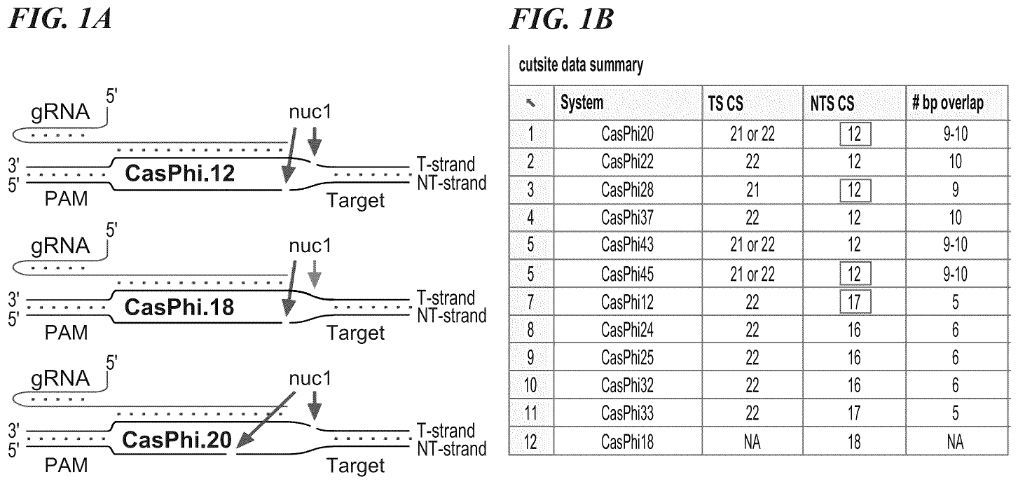

Without limiting ourselves to any particular method or mechanism, effector proteins of the instant disclosure may cleave the non-target strand and/or the target strand of a dsDNA target nucleic acid, see, e.g., FIG. 1A. A ribonucleic acid protein (RNP) complex of the effector protein and guide nucleic acid recognizes a PAM on the target DNA and binds the dsDNA target nucleic acid. A spacer sequence of the guide nucleic acid hybridizes to a target sequence of the dsDNA target nucleic acid. The RNP nicks the target strand and/or the non-target strand of the dsDNA target nucleic acid and the strands of the target DNA dissociate at, or near, the location of nicking. Different effector proteins may nick the nontarget and target strands at different locations and thereby cause an overlap of the target and non-target strands (see, e.g., FIG. 1B). For example, CasPhi.12 (SEQ ID NO: 12) cleaves the non-target strand at 17 bps 3′ of the PAM sequence (that is located on the target strand) and cleaves the target strand at 22 bps 3′ of the PAM sequence. This results in a 5 bp overlap. Also, for example, CasPhi. 18 (SEQ ID NO: 18) may nick only the non-target strand.

### Example 2

An extended guide RNA (rtgRNA) is oriented starting at the 5′ end with a template sequence which is complementary to the target sequence on the non-target strand with the exception of one or more nucleotides conveying an intended genomic edit, and a primer binding sequence (PBS). The template sequence may be referred to as an “RT template.” Located 3′ of the PBS, there is a linker connecting the PBS to the protein binding region comprising a repeat sequence. Lastly, there is the spacer sequence that targets the extended guide RNA (rtgRNA) and an effector protein-RDDP fusion protein to the target sequence (e.g., genomic DNA). See, e.g., FIG. 3. The effector protein nicks the non-target strand of the target nucleic acid and the RDDP synthesizes new DNA off of the nicked end, wherein the new DNA is complementary to the RT template, thereby producing the desired genomic edit in the target nucleic acid.

### Example 3

An extended guide RNA (rtgRNA) is oriented starting at the 5′ end with a protein binding region comprising a repeat sequence, followed by a region comprising a spacer sequence that hybridizes to a target sequence of DNA. Located 3′ of the region comprising the spacer sequence is a template RNA sequence (e.g., an “RT template” or “RTT”) that is complementary to the target sequence on the non-target strand with the exception of one or more nucleotides conveying an intended edit of the target nucleic acid (e.g., genomic DNA). Located 3′ of the RT template is a primer binding sequence (PBS). The effector protein is fused to RDDP so that when the extended guide RNA complexes with the target sequence the effector protein and the RDDP are both localized to the target sequence of the target nucleic acid. See, e.g., FIG. 4. The effector protein nicks the non-target strand of the target nucleic acid and the RDDP synthesizes new DNA off of the nicked end, wherein the new DNA is complementary to the RT template, thereby producing the desired genomic edit in the target nucleic acid.

### Example 4

A gene editing system comprises two separate RNAs and/or two separate proteins. Such systems may be referred to as split protein/RNA design systems. The system comprises a guide RNA, which comprises a spacer sequence that is complementary to a target sequence on a target strand of a target nucleic acid (e.g., genomic DNA, dsDNA molecule). The guide RNA also comprises a protein binding region comprising a repeat sequence. The protein binding region is bound by an effector protein. The repeat sequence may be directly or indirectly 5′ of the spacer sequence. In other instances, the repeat sequence may be directly or indirectly 3′ of the spacer sequence.

The system also comprises a template RNA (rttRNA). The rttRNA comprises a primer binding sequence (PBS) and a template sequence. The template sequence may be an RT template. The rttRNA comprises an MS2 localization sequence. The MS2 localization sequence may be located at the 5′ or 3′ terminus of the rttRNA. The template sequence is complementary to the target sequence on the non-target strand with the exception of one or more nucleotides conveying an intended edit of the target nucleic acid.

The effector protein is not covalently linked to the RDDP, but the RDDP is fused to an MS2 coat protein that binds the MS2 localization sequence, thereby localizing the RDDP to the target nucleic acid. See, e.g., FIGS. 5A-5B. Alternatively, the guide RNA and rttRNA are linked while the effector protein and RDDP are not linked. Also, alternatively, the effector protein and RDDP are linked, while the guide RNA and rttRNA are not linked. The effector protein nicks the non-target strand of the target nucleic acid and the RDDP synthesizes new DNA off of the nicked end, wherein the new DNA is complementary to the RT template, thereby producing the desired genomic edit in the target nucleic acid.

### Example 5

To test if specific CasPhi proteins can be induced to behave as nickases, spacer lengths were modified and tested in the HEK293T nickase assay.

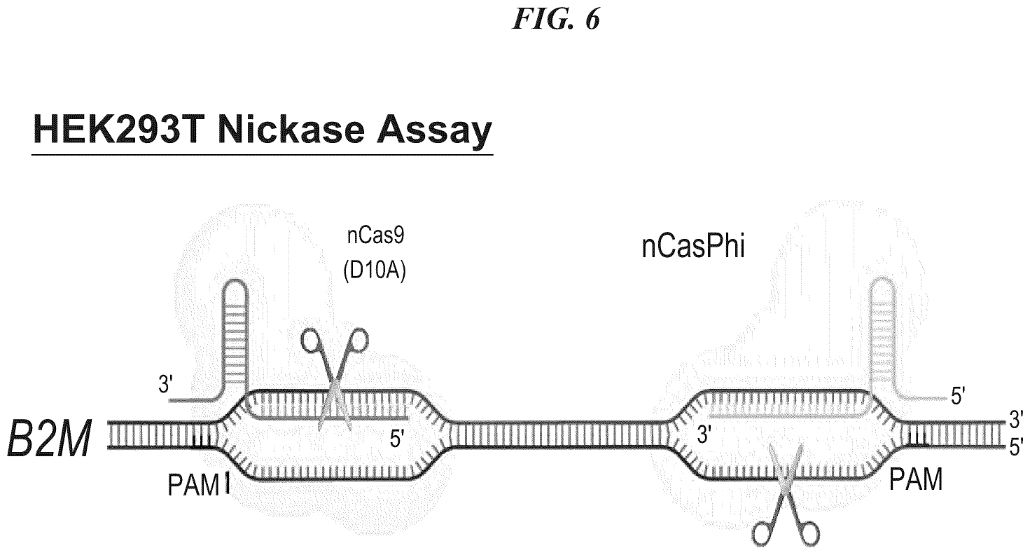

The HEK293T nickase assay uses a previously validated sgRNA/Cas9 pair that nicks one strand of the B2M locus. Indels are only generated if both strands of DNA are nicked. Thus, indels will form only when the sgRNA/Cas9 pair nicks one strand of DNA and a CasPhi protein/guide pair nicks the other strand of the DNA. See FIG. 6 for a depiction of this assay. Thus, indel formation demonstrates CasPhi nickase activity. This is compared to indel formation in the absence of the sgRNA/Cas9 pair to quantify what portion of indels are generated by any residual or accompanying CasPhi nuclease activity.

To further elaborate on the rationale of this assay, if an indel forms when DNA is contacted with a CasPhi protein and guide RNA alone, then the Cas protein in the presence of that guide RNA is acting as a nuclease. If no indel forms in the DNA when the Cas protein and guide RNA are incubated with the Cas9/sgRNA pair with the DNA, then the Cas protein does not have nuclease or nickase activity with that guide RNA. However, if an indel forms only when the Cas protein is incubated in the presence of the guide RNA and the Cas9/sgRNA pair, then the Cas protein in the presence of that guide RNA has nickase activity. Since the only way the indel could have formed was if the Cas protein in the presence of its guide RNA cleaved one strand, and the Cas9/sgRNA pair cleaved the other strand.

In this experiment, different guide lengths were tested to determine which guide lengths could induce nickase activity to a CasPhi nuclease

A Cas9 variant (Cas9 D10A), also denoted as nCas9, shown previously to provide nickase activity, was used as a nickase to cleave one strand of the B2M gene. It was confirmed that nCas9 does not provide double stranded cleavage activity on its own, as evidenced by an absence of indel formation in the B2M gene when the B2M gene was contacted only with nCas9. In contrast, wildtype Cas9 generated indels at the same site. Data from two replicates are provided. See Table 8.

TABLE 8
percent indel formation of nickase
variant nCas9 and wildtype Cas9

| Guide | nCas9 | WT Cas9 |
|---|---|---|
| guide 1 | 0% | 42%, 43% |
| guide 2 | 0% | 33.8%, 47.9% |

### Results

Table 9 provides the results of the above-described assay for combinations of nCas9 and CasPhi.12 (SEQ ID NO: 12) tested for indel formation in B2M gene. The first column of Table 9 shows the various CasPhi. 12 gRNA spacer lengths that were tested. The second column of Table 9 provides data from an experimental control in which no gRNA was provided for nCas9. Thus, any indel formation is generated by CasPhi.12. The second column of Table 9 shows that when CasPhi.12 gRNA spacer length was reduced to 13 to 16 nucleotides, indel formation by CasPhi.12 alone was greatly reduced, nearly negligible as compared to indel formation when the spacer length was 20 nucleotides. Interestingly, as evidenced by the data in the third and fourth column of Table 9, indel formation produced by the CasPhi.12 with a gRNA comprising a 15-nucleotide spacer increased from 1.64% to 12.64% or 14.08% in the presence of nCas9 with two different gRNA. Although more modest, an increase was also observed when the CasPhi.12 gRNA comprised a spacer having a length of 13, 14 or 16 nucleotides.

TABLE 9
Indel formation produced by a combination of a Cas9
nickase system and a CasPhi nickase system.

| | | nCas9 and guide1 | nCas9 and guide 2 |
|---|---|---|---|
| | nCas9 without | (24546) + | (24553) + |
| CasPhi.12 spacer | gRNA + CasPhi.12 | CasPhi.12 with | CasPhi.12 with |
| length (nt) | with B2M gRNA | B2M gRNA | B2M gRNA |
| 20 | 12.85% | 19.52% | 31.795% |
| 16 | 0.075% | 0.155% | 0.68% |
| 15 | 1.64% | 12.64% | 14.08% |
| 14 | 0.035% | 1.38 | 2.555% |
| 13 | 0.01% | 0.59% | 1.365% |

These data demonstrate that a CasPhi. 12 system provides nickase activity on dsDNA.

### Example 6

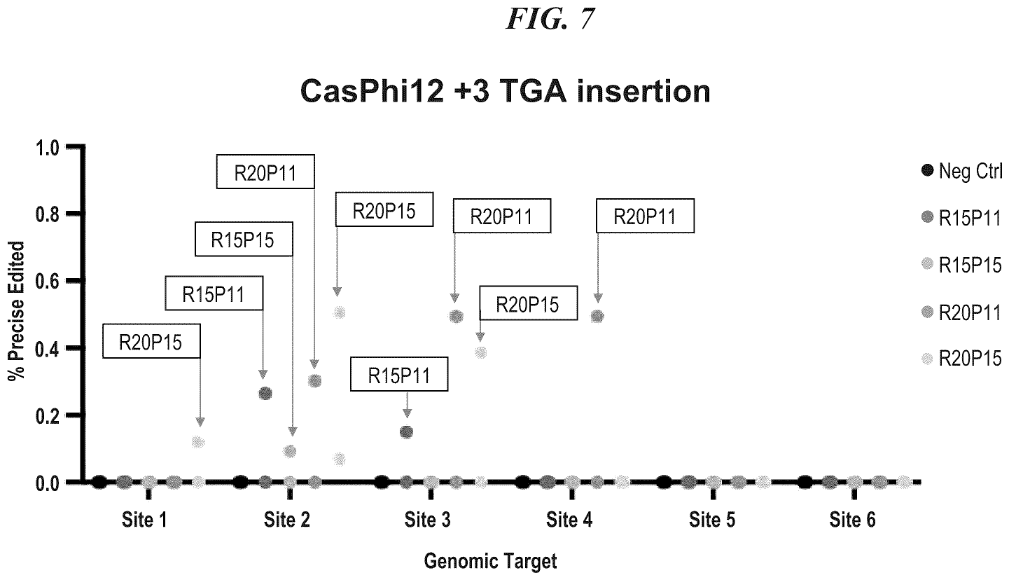

This Example demonstrates the ability of CasPhi. 12, representative of Type V Cas systems, to perform precise editing. This experiment employed a split protein design and split RNA design, as shown in FIG. 5B. Components of the system included: CasPhi.12 effector protein; reverse trancriptase; one of several guide nucleic acids comprising a repeat sequence located 5′ of a 17-20 nt spacer sequence; and a template RNA (rttRNA) comprising in order from 5′ to 3′ an MS2 aptamer, a primer binding sequence and an RT template. The rttRNA comprised an encoded edit of a 3-nucleotide insertion (5′-TGA-3′) relative to the target sequence. HEK293T cells were transiently transfected with these components. NGS revealed that the insertion was obtained with this system. Data is provided in FIG. 7.

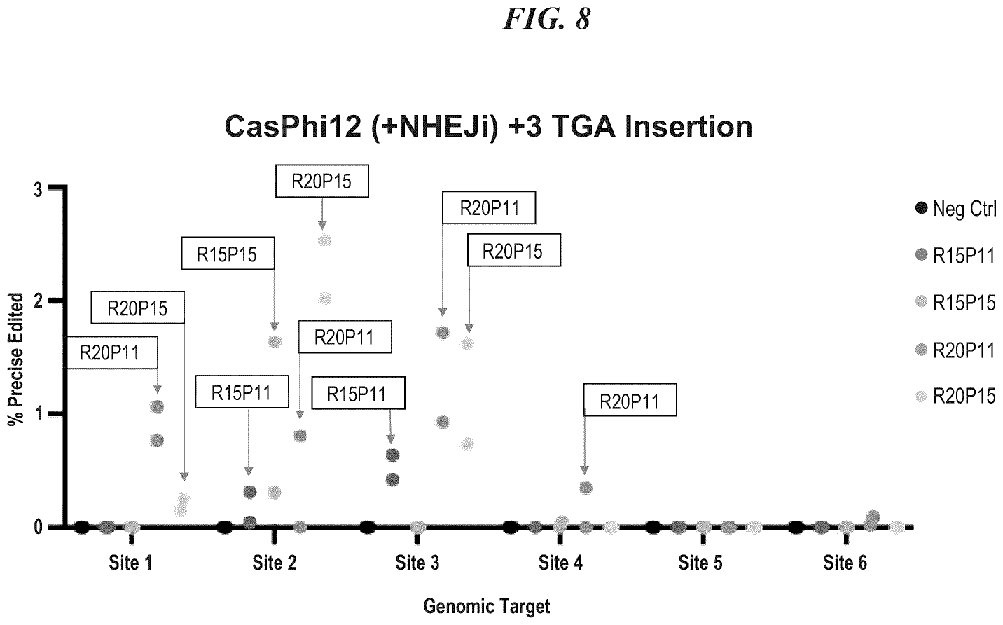

The foregoing experiment was repeated with the addition of non-homologous end joining (NHEJ) inhibitor, AZD7648. NHEJ inhibition increased CasPhi.12 precision editing approximately 5-fold relative to editing in the absence of an NHEJ inhibitor. Data is provided in FIG. 8.

### Example 7

This Example demonstrates the ability of CasM.265466, a protein representative of Cas proteins in the 400-500 amino acid size range, to perform precise editing of double stranded genomic DNA. This experiment employed a split protein design and split RNA design, as shown in FIG. 9.

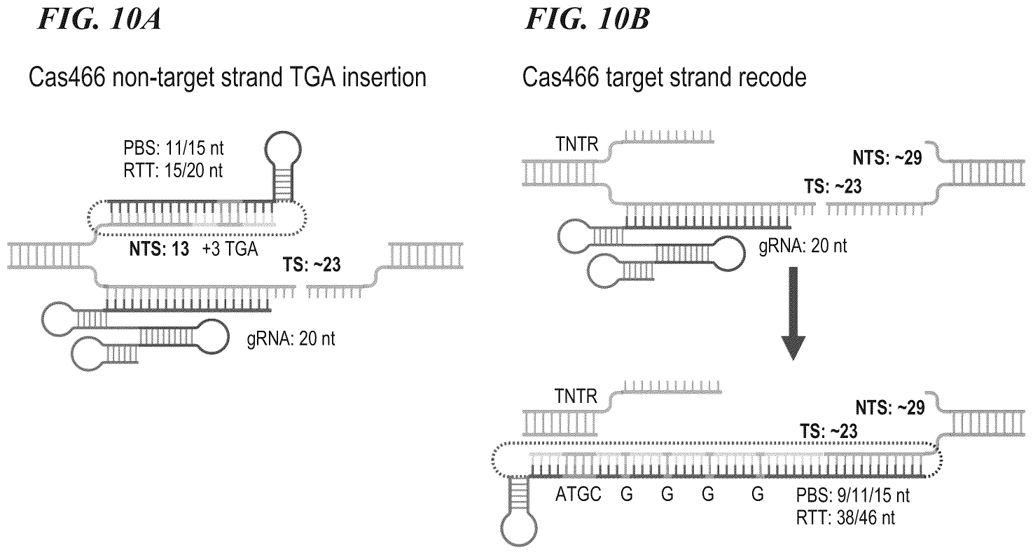

For the non-target strand (NTS), cleavage occurs approximately 29 nt 3′ of the beginning of the spacer (or in other words 29 nt 3′ of the 5′ end of the target sequence on the NTS), then degrades DNA in a 3′->5′ direction until nucleotide 13. The PBS hybridizes to genomic DNA 5′ of nucleotide 13, and a TGA insertion is encoded by the RT template (RTT) 3 nt after nucleotide 13. Editing of the NTS is represented in FIG. 10A.

For the target strand (TS), cleavage occurs approximately at position 23 relative to the 3′ end of the PAM. The PBS hybridizes to NTS 5′ of nucleotide 23, and the precise edit is encoded by the RTT. The precise edit consists of a 4-letter ATGC substitution for the TNTR PAM of CasM.265466, along with several single base C substitutions interspersed between the PAM and PBS. Editing of the TS is represented in FIG. 10B.

Briefly, HEK293T were transfected with 300 ng of a first plasmid encoding CasM.265466 and guide RNA and 100 ng of a second plasmid encoding M-MLV reverse transcriptase and a retRNA (also referred to herein as an rttRNA) using Transit 293T lipofection reagent at a ratio of 3:1 DNA.

Six different guide RNAs corresponding to six different target sites were tested. The guide RNAs comprised a handle sequence and a spacer sequence, wherein the spacer sequence was located 3′ of the handle sequence. gRNA spacer sequences are provided in Table 10. The handle sequence was: 5′-ACAGCUUAUUUGGAAGCUGAAAUGUGAGGUUUAUAACACUCACAAGAAUCCUGA AAAAGGAUGCCAAAC-3′ (SEQ ID NO: 348).

Four different retRNA designs (2 PBS×2 RTT lengths) were tested for targeting the NTS and six different retRNA designs (3 PBS×2 RTT lengths) were tested for targeting the TS. The variable portion of the retRNAs for the NTS are provided in Table 11. The variable portion of the retRNAs for the TS are provided in Table 12. All retRNAs used in this experiment comprised the following sequence, wherein [X] represents the RTT-PBS sequences in Table 11 and Table 12.

5′GUGCUCGCUUCGGCAGCACAUAUACUAGUCGACGGGCCGCACUCGCCGGU CCCAAGCCCGGAUAAAAUGGGAGGGGGCGGGAAACCGCCUaaccaugccgagugcggccgcA ACUAAGCACAUGAGGAUCACCCAUGUGC[X]AAAUUAACAGUGGCCGCGGUCGGC gacuucgguccgc-3′, wherein the bold, italicized, upper case letters represent a 5′ ribozyme sequence (SEQ ID NO: 418), wherein the bold, italicized, lower case letters represent a 5′ homology arm (SEQ ID NO: 419), wherein the bold, non-italicized, upper case letters represent an MS2 loop (SEQ ID NO: 420), wherein the non-bold, italicized, upper case letters represent a 3′ homology arm (SEQ ID NO: 421), and wherein the non-bold, italicized, lower case letters represent a 3′ ribozyme sequence (SEQ ID NO: 422). Remaining sequence represents linker nucleotides. The ribozymes are self-cleaving and generate exposed ends recognized by endogenous RtcB ligase. The ligase “seals” the two exposed ends, creating a circular RNA from the originally linear transcript. Without being bound by theory, this renders the RNA more resistant to degradation inside cells by endogenous RNases. Nucleotides that are recoded from the underlying genomic sequence are represented by lower case letters in Table 12. The reason to have this for the TS and not the NTS is that the direction of DNA synthesis on the TS is such that the PAM of a Type V nuclease can be edited. By editing the PAM, one can prevent re-engagement of the target by the Cas protein after the precise edit is generated and prevent subsequent NHEJ-based indels from making further modifications to the target that could turn the precise edit into an undesirable byproduct.

Cells were incubated 72 hours and harvested for NGS sequence analysis. Each condition was tested in the presence and absence of NHEJ inhibitor (NHEJi) AZD7648. A retRNA with no sequence similarity to a target site served as a negative control.

TABLE 10
Exemplary CasM.265466 Guide RNA Spacer Sequences

| Targeting | | |
|---|---|---|
| Site | Guide RNA Spacer | SEQ ID NO: |
| Site 1 | ACCAAAAAAUAUACGCUAUA | 350 |
| Site 2 | AAGAUAGAGAUAAACCUUUG | 351 |
| Site 3 | AAAUGUUGUGUGUACAUGCU | 352 |
| Site 4 | AGAUAACGUUUGGAAGUUAC | 353 |
| Site 5 | CUGACAAAGAUAUCACUCUG | 354 |
| Site 6 | ACAAUGTGGAUACUUUGUUU | 355 |

TABLE 11
RTT/PBS sequences for non-target strand

| | | | Full retRNA |
|---|---|---|---|
| retRNA ID | RTT/PBS | SEQ ID NO: | SEQ ID NO: |
| Neg control | GGGUCUUCGAGGACGACGAAGAAGA | 356 | 423 |
| | CCU | | |
| Site 1 | AGAGAUAUAUCAGCGUAUAUUUUUUG | 357 | 424 |
| R15P11 | | | |
| Site 1 | AGAGAUAUAUCAGCGUAUAUUUUUUG | 358 | 425 |
| R15P15 | GUUA | | |
| Site 1 | AUUAUAGAGAUAUAUCAGCGUAUAUU | 359 | 426 |
| R20P11 | UUUUG | | |
| Site 1 | AUUAUAGAGAUAUAUCAGCGUAUAUU | 360 | 427 |
| R20P15 | UUUUGGUUA | | |
| Site 2 | UAACACAAAUCAGGUUUAUCUCUAUC | 361 | 428 |
| R15P11 | | | |
| Site 2 | UAACACAAAUCAGGUUUAUCUCUAUC | 362 | 429 |
| R15P15 | UUUA | | |
| Site 2 | GUCAAUAACACAAAUCAGGUUUAUCU | 363 | 430 |
| R20P11 | CUAUC | | |
| Site 2 | GUCAAUAACACAAAUCAGGUUUAUCU | 364 | 431 |
| R20P15 | CUAUCUUUA | | |
| Site 3 | CACCUAGCAUCAUGUACACACAACAU | 365 | 432 |
| R15P11 | | | |
| Site 3 | CACCUAGCAUCAUGUACACACAACAU | 366 | 433 |
| R15P15 | UUUA | | |
| Site 3 | AUACACACCUAGCAUCAUGUACACAC | 367 | 434 |
| R20P11 | AACAU | | |
| Site 3 | AUACACACCUAGCAUCAUGUACACAC | 368 | 435 |
| R20P15 | AACAUUUUA | | |
| Site 4 | UAAAUGUAAUCACUUCCAAACGUUAU | 369 | 436 |
| R15P11 | | | |
| Site 4 | UAAAUGUAAUCACUUCCAAACGUUAU | 370 | 437 |
| R15P15 | CUCA | | |
| Site 4 | CAUUUUAAAUGUAAUCACUUCCAAAC | 371 | 438 |
| R20P11 | GUUAU | | |
| Site 4 | CAUUUUAAAUGUAAUCACUUCCAAAC | 372 | 439 |
| R20P15 | GUUAUCUCA | | |
| Site 5 | ACUAUCAGAUCAGUGAUAUCUUUGUC | 373 | 440 |
| R15P11 | | | |
| Site 5 | ACUAUCAGAUCAGUGAUAUCUUUGUC | 374 | 441 |
| R15P15 | AGUA | | |
| Site 5 | AGGAAACUAUCAGAUCAGUGAUAUCU | 375 | 442 |
| R20P11 | UUGUC | | |
| Site 5 | AGGAAACUAUCAGAUCAGUGAUAUCU | 376 | 443 |
| R20P15 | UUGUCAGUA | | |
| Site 6 | UUGCUAAACUCAAAAGUAUCCACAUU | 377 | 444 |
| R15P11 | | | |
| Site 6 | UUGCUAAACUCAAAAGUAUCCACAUU | 378 | 445 |
| R15P15 | GUUA | | |
| Site 6 | AUGUAUUGCUAAACUCAAAAGUAUCC | 379 | 446 |
| R20P11 | ACAUU | | |
| Site 6 | AUGUAUUGCUAAACUCAAAAGUAUCC | 380 | 447 |
| R20P15 | ACAUUGUUA | | |

TABLE. 12
RTT/PBS sequences for target strand:

| | | | Full retRNA |
|---|---|---|---|
| retRNA ID | RTT/PBS | SEQ ID NO: | SEQ ID NO: |
| Neg control | GGGUCUUCGAGGACGACGAAGAAGAC | 381 | 448 |
| | CU | | |
| Site 1 | UAUCUUUGUCAGaugcACCgAAAgAUA | 382 | 449 |
| R38P09 | UgCGCUgUAUCUCUAUAAUC | | |
| Site 1 | CAGAGUGAUAUCUUUGUCAGaugcACC | 383 | 450 |
| R46P09 | gAAAgAUAUgCGCUgUAUCUCUAUAAU | | |
| | C | | |
| Site 1 | UAUCUUUGUCAGatgcACCgAAAgAUAU | 384 | 451 |
| R38P11 | gCGCUgUAUCUCUAUAAUCUG | | |
| Site 1 | CAGAGUGAUAUCUUUGUCAGaugcACC | 385 | 452 |
| R46P11 | gAAAgAUAUgCGCUgUAUCUCUAUAAU | | |
| | CUG | | |
| Site 1 | UAUCUUUGUCAGaugcACCgAAAgAUA | 386 | 453 |
| R38P15 | UgCGCUgUAUCUCUAUAAUCUGUUUU | | |
| Site 1 | CAGAGUGAUAUCUUUGUCAGaugcACC | 387 | 454 |
| R46P15 | gAAAgAUAUgCGCUgUAUCUCUAUAAU | | |
| | CUGUUUU | | |
| Site 2 | UUUGUUUCAAUaugcAAGgUAGgGAUAg | 388 | 455 |
| R38P09 | ACCUUgGUGUUAUUGACUG | | |
| Site 2 | AGAGAAAAUUUGUUUCAAUaugcAAGg | 389 | 456 |
| R46P09 | UAGgGAUAgACCUUgGUGUUAUUGAC | | |
| | UG | | |
| Site 2 | UUUGUUUCAAUaUgcAAGgUAGgGAUA | 390 | 457 |
| R38P11 | gACCUUgGUGUUAUUGACUGUG | | |
| Site 2 | AGAGAAAAUUUGUUUCAAUaugcAAGg | 391 | 458 |
| R46P11 | UAGgGAUAgACCUUgGUGUUAUUGAC | | |
| | UGUG | | |
| Site 2 | UUUGUUUCAAUaugcAAGUAGgGAUAg | 392 | 459 |
| R38P15 | ACCUUgGUGUUAUUGACUGUGCAAA | | |
| Site 2 | AGAGAAAAUUUGUUUCAAUaugcAAGg | 393 | 460 |
| R46P15 | UAGgGAUAgACCUUgGUGUUAUUGAC | | |
| | UGUGCAAA | | |
| Site 3 | AUUGAGUAUUUUaugcAAgUGUgGUGg | 394 | 461 |
| R38P09 | GUAgAUGgUAGGUGUGUAUA | | |
| Site 3 | GCUUUUAUAUUGAGUAUUUUaugcAAg | 395 | 462 |
| R46P09 | UGUgGUGgGUAgAUGgUAGGUGUGUA | | |
| | UA | | |
| Site 3 | AUUGAGUAUUUUaugcAAgUGUgGUGg | 396 | 463 |
| R38P11 | GUAgAUGgUAGGUGUGUAUAUU | | |
| Site 3 | GCUUUUAUAUUGAGUAUUUUaugcAAg | 397 | 464 |
| R46P11 | UGUgGUGgGUAgAUGgUAGGUGUGUA | | |
| | UAUU | | |
| Site 3 | AUUGAGUAUUUUaugcAAgUGUgGUGg | 398 | 465 |
| R38P15 | GUAgAUGgUAGGUGUGUAUAUUAAUU | | |
| Site 3 | GCUUUUAUAUUGAGUAUUUUaugcAAg | 399 | 466 |
| R46P15 | UGUgGUGgGUAgAUGgUAGGUGUGUA | | |
| | UAUUAAUU | | |
| Site 4 | AAGCAACAUAAAaugcAGAUAACGUUU | 400 | 467 |
| R38P09 | GGAAGUUACAUUUAAAAUGU | | |
| Site 4 | UUUUAAAUAAGCAACAUAAAaugcAGA | 401 | 468 |
| R46P09 | UAACGUUUGGAAGUUACAUUAAAAU | | |
| | GU | | |
| Site 4 | AAGCAACAUAAAaugcAGAUgACGUgU | 402 | 469 |
| R38P11 | GGgAGUUgCAUUUAAAAUGUCU | | |
| Site 4 | UUUUAAAUAAGCAACAUAAAaugcAGA | 403 | 470 |
| R46P11 | UgACGUgUGGgAGUUgCAUUUAAAAUG | | |
| | UCU | | |
| Site 4 | AAGCAACAUAAAaugcAGAUgACGUgU | 404 | 471 |
| R38P15 | GGgAGUUgCAUUUAAAAUGUCUCCUC | | |
| Site 4 | UUUUAAAUAAGCAACAUAAAaugcAGA | 405 | 472 |
| R46P15 | UgACGUgUGGgAGUUgCAUUUAAAAUG | | |
| | UCUCCUC | | |
| Site 5 | AUAUUUUUUGGUaugcCUGgCAAgGAUg | 406 | 473 |
| R38P09 | UCACUgUGAUAGUUUCCUG | | |
| Site 5 | UAUAGCGUAUAUUUUUUGGUaugcCUG | 407 | 474 |
| R46P09 | gCAAgGAUgUCACUgUGAUAGUUUCCU | | |
| | G | | |
| Site 5 | AUAUUUUUUGGUaugcCUGgCAAgGAUg | 408 | 475 |
| R38P11 | UCACUgUGAUAGUUUCCUGCA | | |
| Site 5 | UAUAGCGUAUAUUUUUUGGUaugcCUG | 409 | 476 |
| R46P11 | gCAAgGAUgUCACUgUGAUAGUUUCCU | | |
| | GCA | | |
| Site 5 | AUAUUUUUUGGUaugcCUGgCAAgGAUg | 410 | 477 |
| R38P15 | UCACUgUGAUAGUUUCCUGCAUUUG | | |
| Site 5 | UAUAGCGUAUAUUUUUUGGUaugcCUG | 411 | 478 |
| R46P15 | gCAAgGAUgUCACUgUGAUAGUUUCCU | | |
| | GCAUUUG | | |
| Site 6 | UAUAUAUCUUUUaugcACAgUGUGGgU | 412 | 479 |
| R38P09 | ACUgUGUgUAGCAAUACAUG | | |
| Site 6 | CAUGACAUUAUAUAUCUUUUaugcACA | 413 | 480 |
| R46P09 | gUGUGGgUACUgUGUgUAGCAAUACAU | | |
| | G | | |
| Site 6 | UAUAUAUCUUUUaugcACAgUGUGGgU | 414 | 481 |
| R38P11 | ACUgUGUgUAGCAAUACAUGGU | | |
| Site 6 | CAUGACAUUAUAUAUCUUUUaugcACA | 415 | 482 |
| R46P11 | gUGUGGgUACUgUGUgUAGCAAUACAU | | |
| | GGU | | |
| Site 6 | UAUAUAUCUUUUaugcACAgUGUGGgU | 416 | 483 |
| R38P15 | ACUgUGUgUAGCAAUACAUGGUAGAA | | |
| Site 6 | CAUGACAUUAUAUAUCUUUUaugcACA | 417 | 484 |
| R46P15 | gUGUGGgUACUgUGUgUAGCAAUACAU | | |
| | GGUAGAA | | |

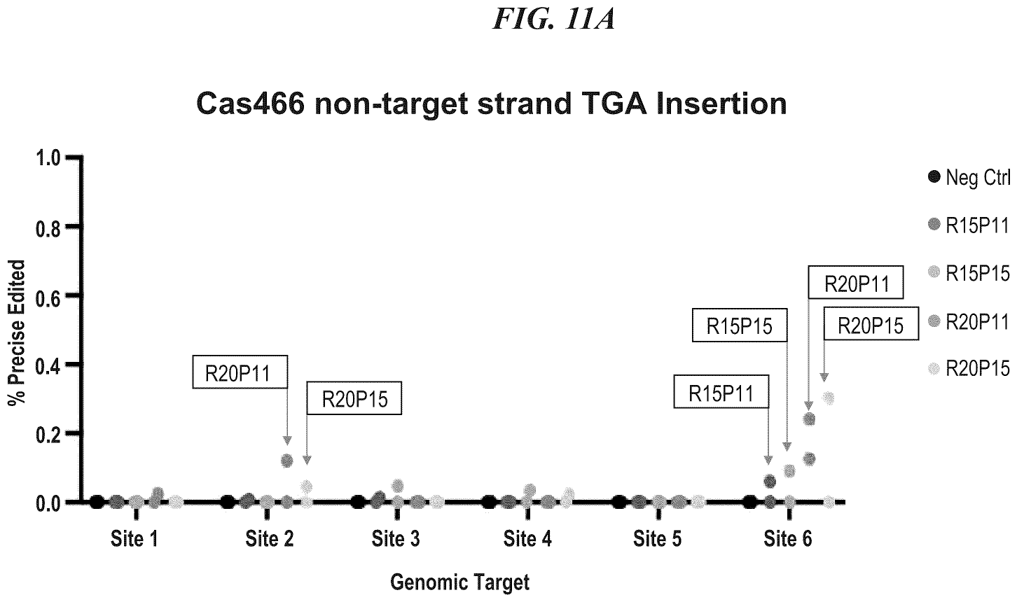

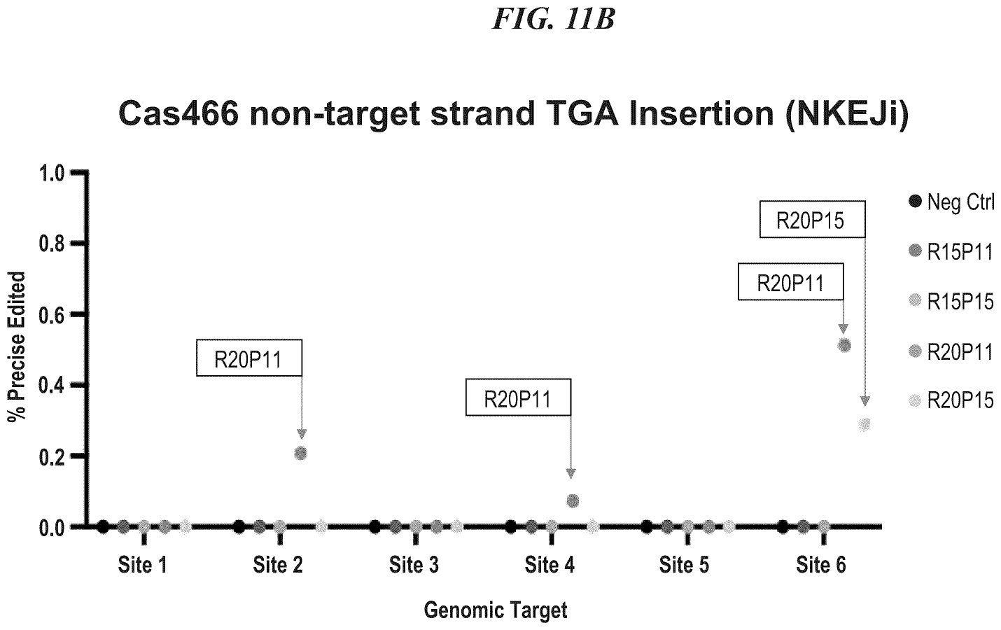

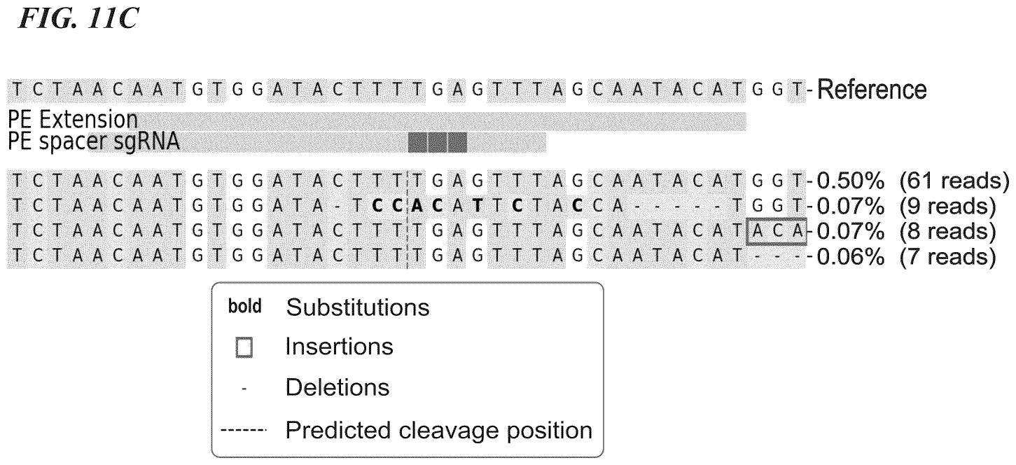

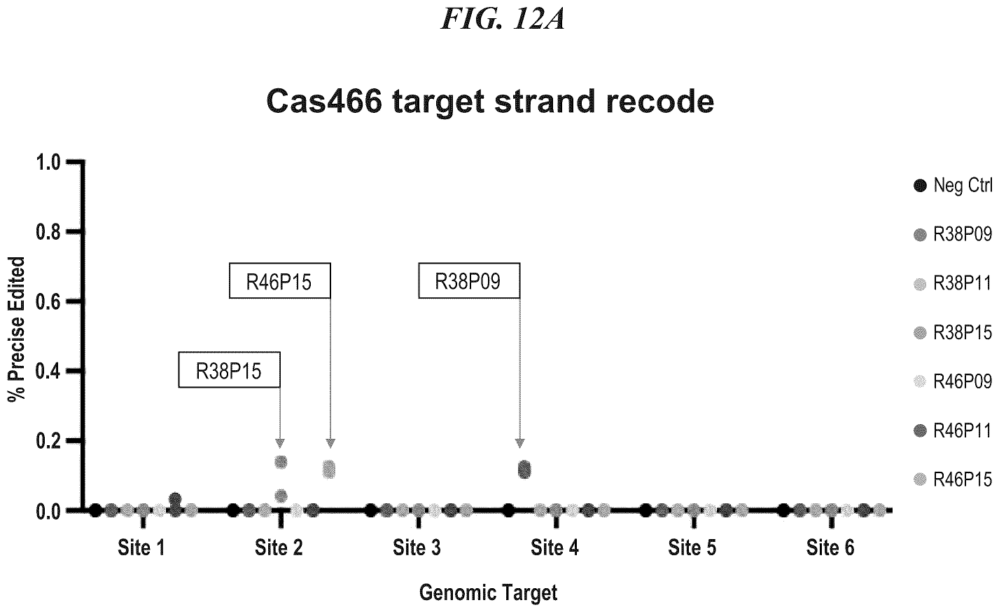

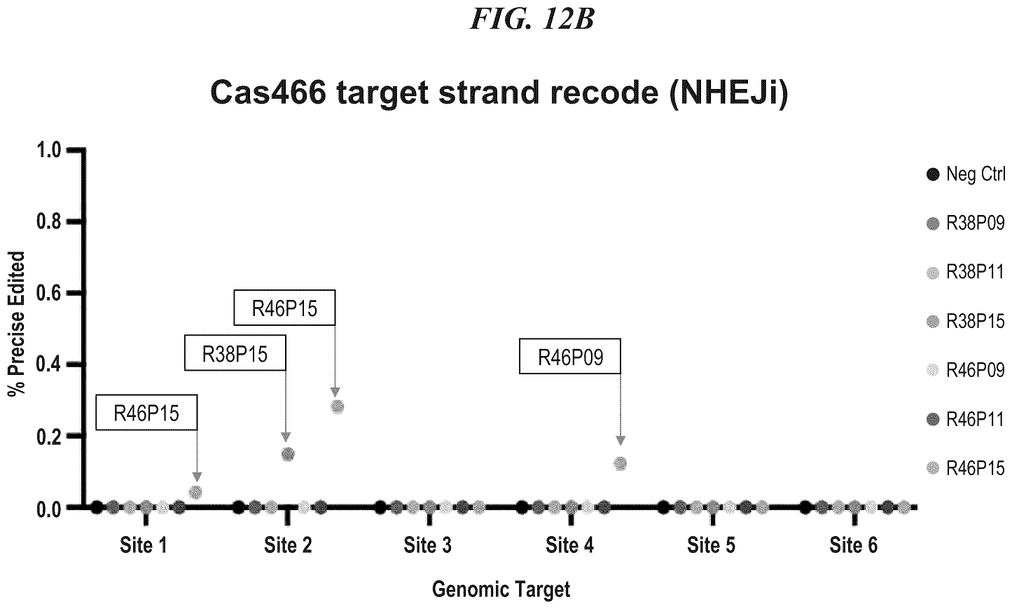

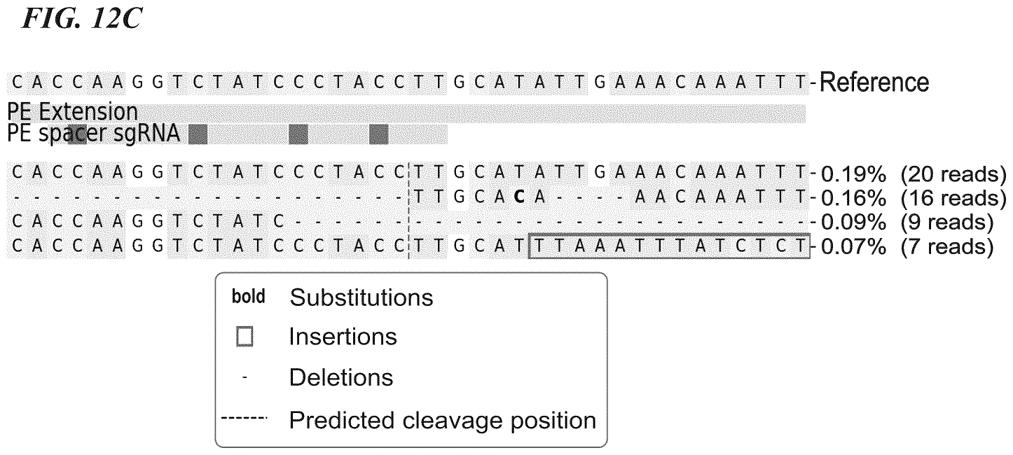

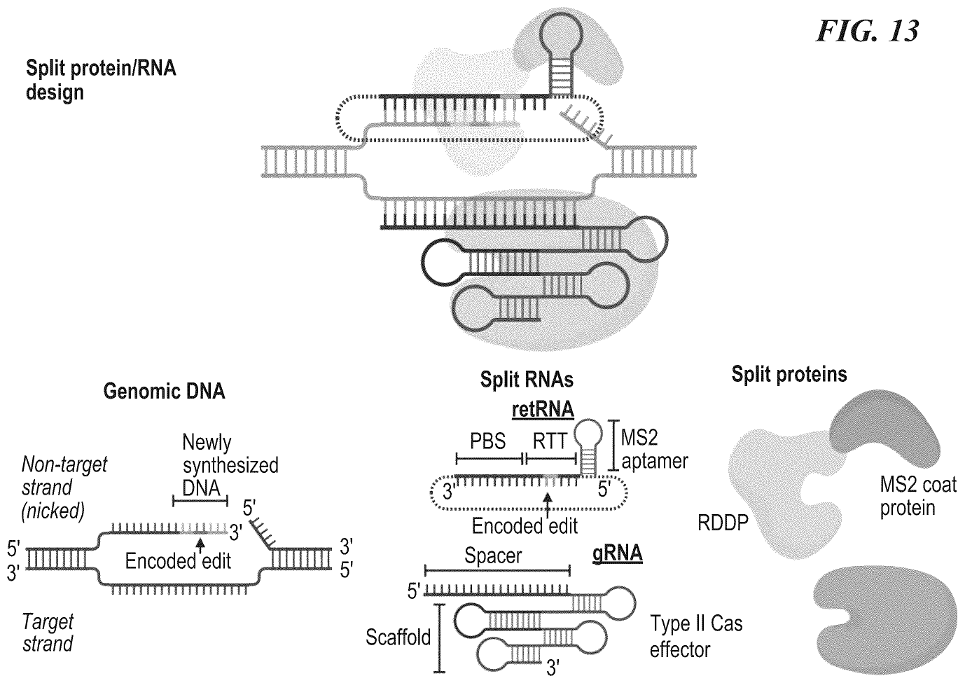

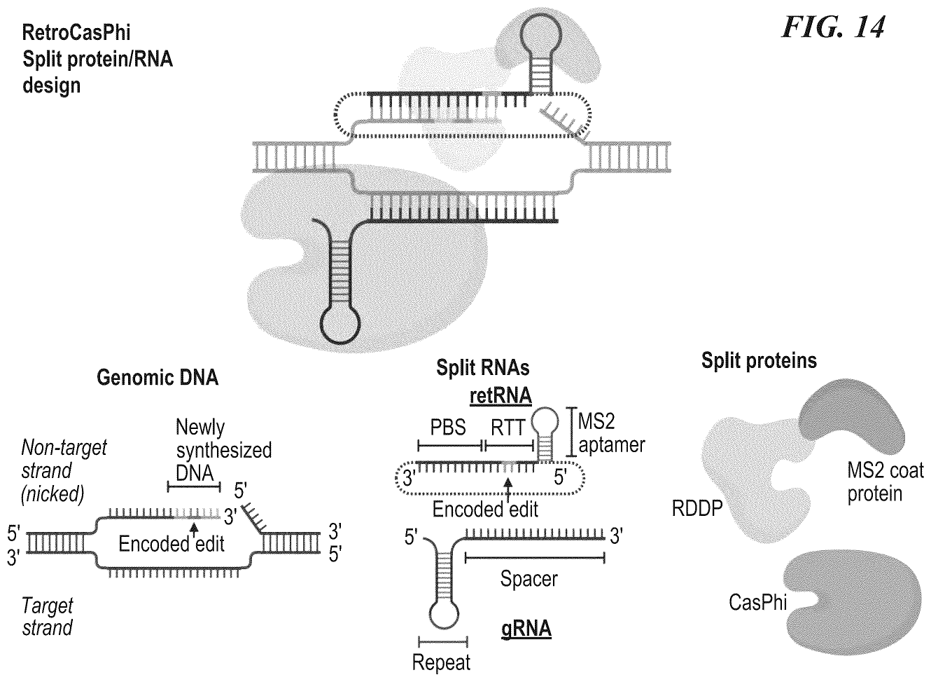

The percentage of target sites on the NTS and TS that were edited precisely in the population of HEK293T cells was assessed by analyzing NGS data. The percentage of target sites on the NTS that were edited precisely is shown in FIG. 11A (without NHEJi) and FIG. 11B (with NHEJi). FIG. 11C shows exemplary NGS data from the NTS. The percentage of target sites on the TS that were edited precisely is shown in FIG. 12A (without NHEJi) and FIG. 12B (with NHEJi). FIG. 12C shows exemplary NGS data from the TS.

### Example 8—Novel RNA-Dependent DNA Polymerases for Precision Editing

To identify novel RNA-dependent DNA polymerases (RDDPs) for precision editing, a metagenomic search was performed. 15 RDDP reference sequences (3 prokaryotic group II intron RDDPs and 12 viral RDDPs) were blasted against sequences available in the Integrated Microbial Genomes System and the UniProt Archive. Around 1.3 million sequences were identified through the metagenomic search. These preliminary sequences varied in lengths and % identity to the 15 reference sequences.

Using guided sampling, the 1.3 million preliminary sequences were refined and filtered based on parameters such as (1) reference coverage over subject, (2) KO prediction, (3) host temperature, etc. Eventually, 46 RDDP candidates (23 prokaryotic group II intron RDDPs and 23 viral RDDPs) were identified.

The 46 identified RDDP candidates were further tested in cultured cells using the PE3 system described in Anzalone et al., Nature. 2019 Dec. 5; 576 (7785): 149-157. Briefly, each of the RDDP candidates was fused to nCas9 (H840A) (SEQ ID NO: 544) on the N terminus or the C terminus, separated by an XTEN40 linker (SEQ ID NO: 543). Each fusion protein was tested against three targets in HEK293T cells for precision editing: (1) HEK site 3 for an A insertion at the +1 position relative to the non-target strand cut site, (2) FANCF for a G to T substitution at the +5 position, and (3) RNF2 for a T insertion at the +1 position. Two control RDDPs were included: the wild-type Moloney murine leukemia virus (M-MLV) RDDP (SEQ ID NO: 531) and the evolved M-MLV RDDP (SEQ ID NO: 532). Each PE3 composition comprising the polynucleotide encoding one of the 92 fusion proteins was transfected into HEK293T cells. 3 days post-transfection, the precision editing outcomes were assessed by next generation sequence (NGS). Each test had two independent replicates.

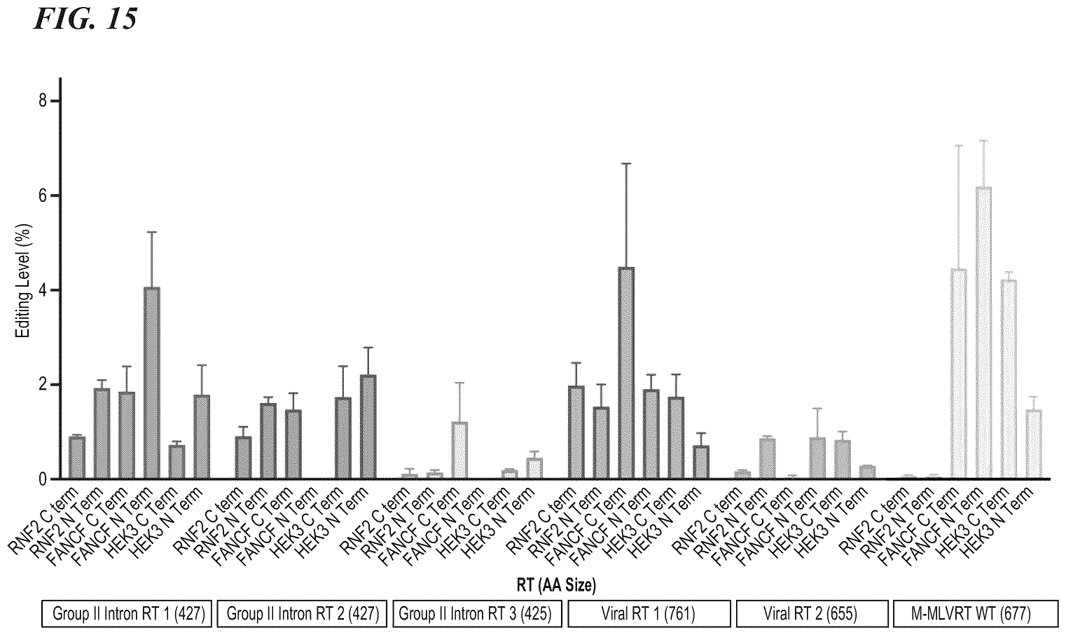

Among the 46 tested RDDP candidates, 5 showed >1% editing efficiency. None of these 5 RDDP candidates have been shown to exhibit reverse polymerase activity in human cells previously. The editing efficiency of the 5 RDDP candidates, as well as that of the wild-type M-MLV RDDP, is shown in FIG. 15. 3 RDDP candidates show editing efficiency comparable to that of the wild-type M-MLV RDDP. Furthermore, the 3 group II intron RDDPs are substantially smaller in size than the wild-type M-MLV RDDP (˜425 aa vs. 677 aa), making them suitable as a component of the precision editing system to be packaged into a single adeno-associated virus (AAV) vector. The amino acid sequences of the N- and C-terminal fusion proteins are as follows in Table A:

TABLE A
Amino acid sequences of N- and C-terminal fusion proteins

| Fusion | | SEQ |
|---|---|---|
| protein | Amino Acid Sequence | ID |
| 2691319- | DKKYSIGLDIGTNSVGWAVITDEYKVPSKKFKVLGNTDRHSIKKNLIGALLFDSGETA | 533 |
| RDDP | EATRLKRTARRRYTRRKNRICYLQEIFSNEMAKVDDSFFHRLEESFLVEEDKKHERHP | |
| fusion at | IFGNIVDEVAYHEKYPTIYHLRKKLVDSTDKADLRLIYLALAHMIKFRGHFLIEGDLN | |
| N term | PDNSDVDKLFIQLVQTYNQLFEENPINASGVDAKAILSARLSKSRRLENLIAQLPGEK | |
| | KNGLFGNLIALSLGLTPNEKSNEDLAEDAKLQLSKDTYDDDLDNLLAQIGDQYADLFL | |
| | AAKNLSDAILLSDILRVNTEITKAPLSASMIKRYDEHHQDLTLLKALVRQQLPEKYKE | |
| | IFFDQSKNGYAGYIDGGASQEEFYKFIKPILEKMDGTEELLVKLNREDLLRKQRTEDN | |
| | GSIPHQIHLGELHAILRRQEDFYPFLKDNREKIEKILTFRIPYYVGPLARGNSRFAWM | |
| | TRKSEETITPWNFEEVVDKGASAQSFIERMTNFDKNLPNEKVLPKHSLLYEYFTVYNE | |
| | LTKVKYVTEGMRKPAFLSGEQKKAIVDLLFKTNRKVTVKQLKEDYFKKIECFDSVEIS | |
| | GVEDRFNASLGTYHDLLKIIKDKDFLDNEENEDILEDIVLTLTLFEDREMIEERLKTY | |
| | AHLFDDKVMKQLKRRRYTGWGRLSRKLINGIRDKQSGKTILDFLKSDGFANRNFMQLI | |
| | HDDSLTFKEDIQKAQVSGQGDSLHEHIANLAGSPAIKKGILQTVKVVDELVKVMGRHK | |
| | PENIVIEMARENQTTQKGQKNSRERMKRIEEGIKELGSQILKEHPVENTQLQNEKLYL | |
| | YYLQNGRDMYVDQELDINRLSDYDVDAIVPQSFLKDDSIDNKVLTRSDKNRGKSDNVP | |
| | SEEVVKKMKNYWRQLLNAKLITQRKFDNLTKAERGGLSELDKAGFIKRQLVETRQITK | |
| | HVAQILDSRMNTKYDENDKLIREVKVITLKSKLVSDFRKDFQFYKVREINNYHHAHDA | |
| | YLNAVVGTALIKKYPKLESEFVYGDYKVYDVRKMIAKSEQEIGKATAKYFFYSNIMNF | |
| | FKTEITLANGEIRKRPLIETNGETGEIVWDKGRDFATVRKVLSMPQVNIVKKTEVQTG | |
| | GFSKESILPKRNSDKLIARKKDWDPKKYGGFDSPTVAYSVLVVAKVEKGKSKKLKSVK | |
| | ELLGITIMERSSFEKNPIDFLEAKGYKEVKKDLIIKLPKYSLFELENGRKRMLASAGE | |
| | LQKGNELALPSKYVNFLYLASHYEKLKGSPEDNEQKQLFVEQHKHYLDEIIEQISEFS | |
| | KRVILADANLDKVLSAYNKHRDKPIREQAENIIHLFTLTNLGAPAAFKYFDTTIDRKR | |
| | YTSTKEVLDATLIHQSITGLYETRIDLSQLGGD | |
| 2691323- | MSELLERILSNDNMNAAYKRVCANKGAGGVDGVTVEELGDYIKENWSSIREQIRRRQY | 534 |
| RDDP | KPQPVRRVEIPKPNGGVRNLGIPTVMDRVIQQGIVQVLSPMCEPLFSERSYGFRPNRS | |
| fusion at | CEMAIRQLLVYLNEGCEWIVDIDLEKFFDNVPQDRLMSLVHDIINDGDTESLIRKYLK | |
| N term | AGVMTPQGYEETKLGTPQGGNLSPLLSNIMLNELDKELEKRGLRFTRYADDCVIAVKS | |
| | ESSAKRVMRTVADWIQRKLGLKVNMTKTHITRPTKLKYLGFGFYRDNKAKEWKCRPHQ | |
| | DSVAKFKRKLKELTCRKTPGTVTGRIAKINQVTRGWINYYAIGSMKTTMIEIDAHLRT | |
| | RLRVIIWKQWKVPKKRQWGLQKLGIGKDLARLTSYYGDRYQWIVTKTCVARAISKEVL | |
| | AKAGLVSCLDYYNERHALKLCGSSGGSPAGSPTSTEEGTSESATPESGPGTSTEPSEG | |
| | SAPGSPAGSGGGSDKKYSIGLDIGTNSVGWAVITDEYKVPSKKFKVLGNTDRHSIKKN | |
| | LIGALLFDSGETAEATRLKRTARRRYTRRKNRICYLQEIFSNEMAKVDDSFFHRLEES | |
| | FLVEEDKKHERHPIFGNIVDEVAYHEKYPTIYHLRKKLVDSTDKADLRLIYLALAHMI | |
| | KFRGHFLIEGDLNPDNSDVDKLFIQLVQTYNQLFEENPINASGVDAKAILSARLSKSR | |
| | RLENLIAQLPGEKKNGLFGNLIALSLGLTPNFKSNFDLAEDAKLQLSKDTYDDDLDNL | |
| | LAQIGDQYADLFLAAKNLSDAILLSDILRVNTEITKAPLSASMIKRYDEHHQDLTLLK | |
| | ALVRQQLPEKYKEIFFDQSKNGYAGYIDGGASQEEFYKFIKPILEKMDGTEELLVKLN | |
| | REDLLRKQRTFDNGSIPHQIHLGELHAILRRQEDFYPFLKDNREKIEKILTFRIPYYV | |
| | GPLARGNSRFAWMTRKSEETITPWNFEEVVDKGASAQSFIERMTNEDKNLPNEKVLPK | |
| | HSLLYEYFTVYNELTKVKYVTEGMRKPAFLSGEQKKAIVDLLFKTNRKVTVKQLKEDY | |
| | FKKIECFDSVEISGVEDRFNASLGTYHDLLKIIKDKDFLDNEENEDILEDIVLTLTLF | |
| | EDREMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHP | |
| | VENTQLQNEKLYLYYLQNGRDMYVDQELDINRLSDYDVDAIVPQSFLKDDSIDNKVLT | |
| | RSDKNRGKSDNVPSEEVVKKMKNYWRQLLNAKLITQRKFDNLTKAERGGLSELDKAGE | |
| | IKRQLVETRQITKHVAQILDSRMNTKYDENDKLIREVKVITLKSKLVSDFRKDFQFYK | |
| | VREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMP | |
| | QVNIVKKTEVQTGGFSKESILPKRNSDKLIARKKDWDPKKYGGFDSPTVAYSVLVVAK | |
| | VEKGKSKKLKSVKELLGITIMERSSFEKNPIDFLEAKGYKEVKKDLIIKLPKYSLFEL | |
| | ENGRKRMLASAGELQKGNELALPSKYVNFLYLASHYEKLKGSPEDNEQKQLFVEQHKH | |
| | YLDEIIEQISEFSKRVILADANLDKVLSAYNKHRDKPIREQAENIIHLFTLTNLGAPA | |
| | AFKYFDTTIDRKRYTSTKEVLDATLIHQSITGLYETRIDLSQLGGD | |
| 2691335- | MLDRDNLRLAYKRVVRNGGAPGVDGVTVAALQSYLNTHWDRVKNELLAGTYRPAPVRR | 535 |
| RDDP | VEIPKPGGGVRLLGIPTVMDRFLQQALLQVMNPIFDAHFSWYSYGFRPGKRAHDAVMQ | |
| fusion at | AQRYIRDGYRWVVDLDLEKFFDRVNHDMLMARVARKVKDKRVLKLIRAYLNAGVMING | |
| N term | VCQRTEEGTPQGGPLSPLLANILLDDLDKELTRRGLRFVRYADDCNIFVASKRAGERV | |
| | MESAIRFLEGKLKLKVNRDKSAVDRPWKRKFLGFSFLSDKEATIRLAPKTISRFKERV | |
| | REITSRSRPIPMEERIRRLNQYTMGWVSYFRLASMKNHCERFDQWIRRRLRMCLWKQW | |
| | KRVRTRIRELRALGVPDWACFAMANSRRGPWEMSRNINNALPTSYWEAKGLKSMLTRY | |
| | MVLRQPFGTAWCGPACQVVGSSGGSPAGSPTSTEEGTSESATPESGPGTSTEPSEGSA | |
| | PGSPAGSGGGSDKKYSIGLDIGTNSVGWAVITDEYKVPSKKFKVLGNTDRHSIKKNLI | |
| | GALLFDSGETAEATRLKRTARRRYTRRKNRICYLQEIFSNEMAKVDDSFFHRLEESEL | |
| | VEEDKKHERHPIFGNIVDEVAYHEKYPTIYHLRKKLVDSTDKADLRLIYLALAHMIKE | |
| | RGHFLIEGDLNPDNSDVDKLFIQLVQTYNQLFEENPINASGVDAKAILSARLSKSRRL | |
| | ENLIAQLPGEKKNGLFGNLIALSLGLTPNFKSNEDLAEDAKLQLSKDTYDDDLDNLLA | |
| | QIGDQYADLFLAAKNLSDAILLSDILRVNTEITKAPLSASMIKRYDEHHQDLTLLKAL | |
| | VRQQLPEKYKEIFFDQSKNGYAGYIDGGASQEEFYKFIKPILEKMDGTEELLVKLNRE | |
| | DLLRKQRTFDNGSIPHQIHLGELHAILRRQEDFYPFLKDNREKIEKILTFRIPYYVGP | |
| | LARGNSRFAWMTRKSEETITPWNFEEVVDKGASAQSFIERMTNFDKNLPNEKVLPKHS | |
| | LLYEYFTVYNELTKVKYVTEGMRKPAFLSGEQKKAIVDLLFKTNRKVTVKQLKEDYFK | |
| | KIECFDSVEISGVEDRENASLGTYHDLLKIIKDKDFLDNEENEDILEDIVLTLTLFED | |
| | REMIEERLKTYAHLFDDKVMKQLKRRRYTGWGRLSRKLINGIRDKQSGKTILDFLKSD | |
| | GFANRNFMQLIHDDSLTFKEDIQKAQVSGQGDSLHEHIANLAGSPAIKKGILQTVKVV | |
| | DELVKVMGRHKPENIVIEMARENQTTQKGQKNSRERMKRIEEGIKELGSQILKEHPVE | |
| | NTQLQNEKLYLYYLQNGRDMYVDQELDINRLSDYDVDAIVPQSFLKDDSIDNKVLTRS | |
| | DKNRGKSDNVPSEEVVKKMKNYWRQLLNAKLITQRKFDNLTKAERGGLSELDKAGFIK | |
| | RQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATA | |
| | KYFFYSNIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPKYSLFELEN | |
| | GRKRMLASAGELQKGNELALPSKYVNFLYLASHYEKLKGSPEDNEQKQLFVEQHKHYL | |
| | DEIIEQISEFSKRVILADANLDKVLSAYNKHRDKPIREQAENIIHLFTLINLGAPAAF | |
| | KYFDTTIDRKRYTSTKEVLDATLIHQSITGLYETRIDLSQLGGD | |
| 2691339- | ENPIKIDFFEPLEIEPLNLIDVNFVEKMDMDIDLSYEQDSLLKQNLKNLRLDHCNKEE | 536 |
| RDDP | KDAIRKLCFDYRDIFYCEQIPLSFTSEITHKIKLNDESPIYTKSYRFPEIHKKEVKDQ | |
| fusion at | IAKMLDQGIIQHSISPWSSPIWVVPKKLDASGRRKWRLVIDYRKLNEKSCNDRYPLPN | |
| N term | ITDILDKLGRANYFTTLDLASGFHQIQVDPDDIPKTAFSTEGGHYEFKRMPFGLKNAP | |
| | STFQRVMDNILRGLHNEICLVYLDDIIIFSTSLDEHIQRLKSVEDRLRKSNFKLQLDK | |
| | SEFLQKTVQYLGHIITPQGVKPNPDKVSTIKRFPIPRTQKDIKSFLGLLGYYRRFIKD | |
| | FAKITKPLTKCLKKNAKVTHDQNFIDAFNTCKEILVNDPILQYPDFSKPFILTTDASD | |
| | VALGAILSQGTLPNDRPVAYASRTLNETESKYSTIEKELLAIVWACKHERPYLYGRKE | |
| | TIYTDHRPLTWLFSLKEPNSKLVRWRLKLEEFEYDIIYKKGKLNTNADCLSRVTLNAI | |
| | DNESMLNNPGDIDQDILDTLQNVTQNLQTLQDFQKPSTSNINDKINIISDIQIKPPDN | |
| | SQNDNTNTSTQHSTANETINDGIKIFDEIINNKTNQILVFPNVIYKLDIKRETYENHK | |
| | IVTVKIPIINNEQMILQFLKENTDPKHVYCMYFQSDELYNDFCTVYLKHFSEKGPKLI | |
| | RCLKLVNTVADKEEQILLIKNHHGSKINHRGINETLEKLKFNYYWKNMKSTVSNFINA | |
| | CDICQRAGSSGGSPAGSPTSTEEGTSESATPESGPGTSTEPSEGSAPGSPAGSGGGSD | |
| | KKYSIGLDIGTNSVGWAVITDEYKVPSKKFKVLGNTDRHSIKKNLIGALLFDSGETAE | |
| | ATRLKRTARRRYTRRKNRICYLQEIFSNEMAKVDDSFFHRLEESFLVEEDKKHERHPI | |
| | FGNIVDEVAYHEKYPTIYHLRKKLVDSTDKADLRLIYLALAHMIKFRGHFLIEGDLNP | |
| | DNSDVDKLFIQLVQTYNQLFEENPINASGVDAKAILSARLSKSRRLENLIAQLPGEKK | |
| | NGLFGNLIALSLGLTPNFKSNFDLAEDAKLQLSKDTYDDDLDNLLAQIGDQYADLFLA | |
| | AKNLSDAILLSDILRVNTEITKAPLSASMIKRYDEHHQDLTLLKALVRQQLPEKYKEI | |
| | FFDQSKNGYAGYIDGGASQEEFYKFIKPILEKMDGTEELLVKLNREDLLRKQRTEDNG | |
| | SIPHQIHLGELHAILRRQEDFYPFLKDNREKIEKILTFRIPYYVGPLARGNSRFAWMT | |
| | RKSEETITPWNFEEVVDKGASAQSFIERMTNFDKNLPNEKVLPKHSLLYEYFTVYNEL | |
| | TKVKYVTEGMRKPAFLSGEQKKAIVDLLFKTNRKVTVKQLKEDYFKKIECFDSVEISG | |
| | VEDRFNASLGTYHDLLKIIKDKDFLDNEENEDILEDIVLTLTLFEDREMIEERLKTYA | |
| | HLFDDKVMKQLKRRRYTGWGRLSRKLINGIRDKQSGKTILDFLKSDGFANRNFMQLIH | |
| | DDSLTFKEDIQKAQVSGQGDSLHEHIANLAGSPAIKKGILQTVKVVDELVKVMGRHKP | |
| | ENIVIEMARENQTTQKGQKNSRERMKRIEEGIKELGSQILKEHPVENTQLQNEKLYLY | |
| | YLQNGRDMYVDQELDINRLSDYDVDAIVPQSFLKDDSIDNKVLTRSDKNRGKSDNVPS | |
| | EEVVKKMKNYWRQLLNAKLITQRKFDNLTKAERGGLSELDKAGFIKRQLVETRQITKH | |
| | VAQILDSRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYSNIMNFF | |
| | KTEITLANGEIRKRPLIETNGETGEIVWDKGRDFATVRKVLSMPQVNIVKKTEVQTGG | |
| | FSKESILPKRNSDKLIARKKDWDPKKYGGFDSPTVAYSVLVVAKVEKGKSKKLKSVKE | |
| | LLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSK | |
| | RVILADANLDKVLSAYNKHRDKPIREQAENIIHLFTLTNLGAPAAFKYFDTTIDRKRY | |
| | TSTKEVLDATLIHQSITGLYETRIDLSQLGGD | |
| 2691355- | VLIRDTSITTNPLPARYHDFSDVFEKKNADRLPEHRLYDCPIDLQEGAHPPFGPIYGL | 537 |
| RDDP | AEPELEALREYLKENLAKGFIQPSKSPAGAPILFVKKKDGSLRLCVDYRGLNRVTVRN | |
| fusion at | RYPLPLIPELLDRLRTGRVFSKIDLRGAYNLVRIKPGDEWKTAFRTRYGHFEYKVMPF | |
| N term | GLTNAPAVFQHMMNHIFREYLDHFVVIYLDDILVFSPSMEEHTRHVRLILTKLREHGL | |
| | YAKSEKCEFDRTSVEFLGYVISPAGITMDPRKVATIHNWPIPTRLKEVQSFLGFANFY | |
| | RRFIDRFSTIVQPLISLTRKDVPFVWTATTQHAFDALKQAFMSAPVLVHPNPAKPFQV | |
| | ETDASDFALGAILSQLDDDGTLHPVAYYSRKFLASEINYPVYDKELAAIISAFAEWRP | |
| | YLVGAQHRIQVVTDHKNLLYFASSRTLNRRQARWSIFLADYDFEIIFRPGIQHGKADA | |
| | LSRRPDLALCPGDDAYTQQSCSLLKPDQLQLSATFMLHDDSLLQEIAQATKTDTFATE | |
| | ILKRLQDSSPATKRADLHHFTTHDGLLYRNHLLYVPEGPCRTQVLQTCHDDPLAGHFG | |
| | VAKTLELVSRGFWWPQPWKLVKEFVKTCDVCARSKAVHHRPYGLLHPLPIPNRPWASI | |
| | SMDFITDLPPVNGVDTVGSSGGSPAGSPTSTEEGTSESATPESGPGTSTEPSEGSAPG | |
| | SPAGSGGGSDKKYSIGLDIGTNSVGWAVITDEYKVPSKKFKVLGNTDRHSIKKNLIGA | |
| | LLFDSGETAEATRLKRTARRRYTRRKNRICYLQEIFSNEMAKVDDSFFHRLEESELVE | |
| | EDKKHERHPIFGNIVDEVAYHEKYPTIYHLRKKLVDSTDKADLRLIYLALAHMIKFRG | |
| | HFLIEGDLNPDNSDVDKLFIQLVQTYNQLFEENPINASGVDAKAILSARLSKSRRLEN | |
| | LIAQLPGEKKNGLFGNLIALSLGLTPNFKSNFDLAEDAKLQLSKDTYDDDLDNLLAQI | |
| | GDQYADLFLAAKNLSDAILLSDILRVNTEITKAPLSASMIKRYDEHHQDLTLLKALVR | |
| | QQLPEKYKEIFFDQSKNGYAGYIDGGASQEEFYKFIKPILEKMDGTEELLVKLNREDL | |
| | LRKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHSLL | |
| | YEYFTVYNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLTLFEDRE | |
| | MIEERLKTYAHLEDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDE | |
| | LVKVMGRHKPENIVIEMARENQTTQKGQKNSRERMKRIEEGIKELGSQILKEHPVENT | |
| | QLQNEKLYLYYLQNGRDMYVDQELDINRLSDYDVDAIVPQSFLKDDSIDNKVLTRSDK | |
| | NRGKSDNVPSEEVVKKMKNYWRQLLNAKLITQRKFDNLTKAERGGLSELDKAGFIKRQ | |
| | LVETRQITKHVAQILDSRMNTKYDENDKLIREVKVITLKSKLVSDERKDFQFYKVREI | |
| | NNYHHAHDAYLNAVVGTALIKKYPKLESEFVYGDYKVYDVRKMIAKSEQEIGKATAKY | |
| | FFYSNIMNFFKTEITLANGEIRKRPLIETNGETGEIVWDKGRDFATVRKVLSMPQVNI | |
| | VKKTEVQTGGFSKESILPKRNSDKLIARKKDWDPKKYGGFDSPTVAYSVLVVAKVEKG | |
| | KSKKLKSVKELLGITIMERSSFEKNPIDFLEAKGYKEVKKDLIIKLPKYSLFELENGR | |
| | KRMLASAGELQKGNELALPSKYVNFLYLASHYEKLKGSPEDNEQKQLFVEQHKHYLDE | |
| | IIEQISEFSKRVILADANLDKVLSAYNKHRDKPIREQAENIIHLFTLINLGAPAAFKY | |
| | FDTTIDRKRYTSTKEVLDATLIHQSITGLYETRIDLSQLGGD | |
| 2691319- | DKKYSIGLDIGTNSVGWAVITDEYKVPSKKFKVLGNTDRHSIKKNLIGALLFDSGETA | 538 |
| RDDP | EATRLKRTARRRYTRRKNRICYLQEIFSNEMAKVDDSFFHRLEESFLVEEDKKHERHP | |
| fusion at | IFGNIVDEVAYHEKYPTIYHLRKKLVDSTDKADLRLIYLALAHMIKFRGHFLIEGDLN | |
| C term | PDNSDVDKLFIQLVQTYNQLFEENPINASGVDAKAILSARLSKSRRLENLIAQLPGEK | |
| | KNGLFGNLIALSLGLTPNFKSNFDLAEDAKLQLSKDTYDDDLDNLLAQIGDQYADLEL | |
| | AAKNLSDAILLSDILRVNTEITKAPLSASMIKRYDEHHQDLTLLKALVRQQLPEKYKE | |
| | IFFDQSKNGYAGYIDGGASQEEFYKFIKPILEKMDGTEELLVKLNREDLLRKQRTFDN | |
| | GSIPHQIHLGELHAILRRQEDFYPFLKDNREKIEKILTFRIPYYVGPLARGNSRFAWM | |
| | TRKSEETITPWNFEEVVDKGASAQSFIERMTNEDKNLPNEKVLPKHSLLYEYFTVYNE | |
| | LTKVKYVTEGMRKPAFLSGEQKKAIVDLLFKTNRKVTVKQLKEDYFKKIECFDSVEIS | |
| | GVEDRFNASLGTYHDLLKIIKDKDELDNEENEDILEDIVLTLTLFEDREMIEERLKTY | |
| | AHLFDDKVMKQLKRRRYTGWGRLSRKLINGIRDKQSGKTILDFLKSDGFANRNFMQLI | |
| | HDDSLTFKEDIQKAQVSGQGDSLHEHIANLAGSPAIKKGILQTVKVVDELVKVMGRHK | |
| | PENIVIEMARENQTTQKGQKNSRERMKRIEEGIKELGSQILKEHPVENTQLQNEKLYL | |
| | YYLQNGRDMYVDQELDINRLSDYDVDAIVPQSFLKDDSIDNKVLTRSDKNRGKSDNVP | |
| | SEEVVKKMKNYWRQLLNAKLITQRKFDNLTKAERGGLSELDKAGFIKRQLVETRQITK | |
| | HVAQILDSRMNTKYDENDKLIREVKVITLKSKLVSDFRKDFQFYKVREINNYHHAHDA | |
| | YLNAVVGTALIKKYPKLESEFVYGDYKVYDVRKMIAKSEQEIGKATAKYFFYSNIMNF | |
| | FKTEITLANGEIRKRPLIETNGETGEIVWDKGRDFATVRKVLSMPQVNIVKKTEVQTG | |
| | GFSKESILPKRNSDKLIARKKDWDPKKYGGFDSPTVAYSVLVVAKVEKGKSKKLKSVK | |
| | ELLGITIMERSSFEKNPIDFLEAKGYKEVKKDLIIKLPKYSLFELENGRKRMLASAGE | |
| | LQKGNELALPSKYVNFLYLASHYEKLKGSPEDNEQKQLFVEQHKHYLDEIIEQISEFS | |
| | KRVILADANLDKVLSAYNKHRDKPIREQAENIIHLFTLTNLGAPAAFKYFDTTIDRKR | |
| | YTSTKEVLDATLIHQSITGLYETRIDLSQLGGDGSSGGSPAGSPTSTEEGTSESATPE | |
| | SGPGTSTEPSEGSAPGSPAGSGGGSMSELLEKILSKDNMNTAYKRVCANKGAGGVDDV | |
| | TVEELGAYVKENWESIREQIRRREYTPQPVRRVEIPKPDGGERKLGIPTVMDRVIQQG | |
| | IVQIISPMCEPLFSKWSYGFRPNRSCEMAIRQLLEYLNEGYEWIVDIDLEKFFDNVPQ | |
| | DRLMSLVHDIIKDGDTESLIRKYLKAGAMTPQGYKETKLGTPQGGNLSPLLSNIMLNE | |
| | LDKELEARGLRFTRYADDCVIAVKSESSAKRVMRTVTDWIQRKLGLKVNMTKSQITRP | |
| | QKLKYLGFGFHKDSKAKEWKCRPHQESVKKFKVRLKELTCRKKPGTVTSKIAKINQVT | |
| | RGWINYYALGSMKTAMTEIDAHLRTRLRVIIWKQWKVPKKRQWGLQKLGVSKDLARLT | |
| | AYCGDSYYWVATKTCVVRAISKEVFAKSGLVSCLDYYNERHALKLC | |
| 2691323- | DKKYSIGLDIGTNSVGWAVITDEYKVPSKKFKVLGNTDRHSIKKNLIGALLFDSGETA | 539 |
| RDDP | EATRLKRTARRRYTRRKNRICYLQEIFSNEMAKVDDSFFHRLEESFLVEEDKKHERHP | |
| fusion at | IFGNIVDEVAYHEKYPTIYHLRKKLVDSTDKADLRLIYLALAHMIKFRGHFLIEGDLN | |
| C term | PDNSDVDKLFIQLVQTYNQLFEENPINASGVDAKAILSARLSKSRRLENLIAQLPGEK | |
| | KNGLFGNLIALSLGLTPNFKSNFDLAEDAKLQLSKDTYDDDLDNLLAQIGDQYADLFL | |
| | AAKNLSDAILLSDILRVNTEITKAPLSASMIKRYDEHHQDLTLLKALVRQQLPEKYKE | |
| | IFFDQSKNGYAGYIDGGASQEEFYKFIKPILEKMDGTEELLVKLNREDLLRKQRTEDN | |
| | GSIPHQIHLGELHAILRRQEDFYPFLKDNREKIEKILTFRIPYYVGPLARGNSRFAWM | |
| | TRKSEETITPWNFEEVVDKGASAQSFIERMTNFDKNLPNEKVLPKHSLLYEYFTVYNE | |
| | LTKVKYVTEGMRKPAFLSGEQKKAIVDLLFKTNRKVTVKQLKEDYFKKIECFDSVEIS | |
| | GVEDRFNASLGTYHDLLKIIKDKDFLDNEENEDILEDIVLTLTLFEDREMIEERLKTY | |
| | AHLFDDKVMKQLKRRRYTGWGRLSRKLINGIRDKQSGKTILDFLKSDGFANRNFMQLI | |
| | HDDSLTFKEDIQKAQVSGQGDSLHEHIANLAGSPAIKKGILQTVKVVDELVKVMGRHK | |
| | PENIVIEMARENQTTQKGQKNSRERMKRIEEGIKELGSQILKEHPVENTQLQNEKLYL | |
| | YYLQNGRDMYVDQELDINRLSDYDVDAIVPQSFLKDDSIDNKVLTRSDKNRGKSDNVP | |
| | SEEVVKKMKNYWRQLLNAKLITQRKFDNLTKAERGGLSELDKAGFIKRQLVETRQITK | |
| | HVAQILDSRMNTKYDENDKLIREVKVITLKSKLVSDFRKDFQFYKVREINNYHHAHDA | |
| | YLNAVVGTALIKKYPKLESEFVYGDYKVYDVRKMIAKSEQEIGKATAKYFFYSNIMNF | |
| | FKTEITLANGEIRKRPLIETNGETGEIVWDKGRDFATVRKVLSMPQVNIVKKTEVQTG | |
| | GFSKESILPKRNSDKLIARKKDWDPKKYGGFDSPTVAYSVLVVAKVEKGKSKKLKSVK | |
| | ELLGITIMERSSFEKNPIDFLEAKGYKEVKKDLIIKLPKYSLFELENGRKRMLASAGE | |
| | LQKGNELALPSKYVNFLYLASHYEKLKGSPEDNEQKQLFVEQHKHYLDEIIEQISEFS | |
| | KRVILADANLDKVLSAYNKHRDKPIREQAENIIHLFTLTNLGAPAAFKYFDTTIDRKR | |
| | YTSTKEVLDATLIHQSITGLYETRIDLSQLGGDGSSGGSPAGSPTSTEEGTSESATPE | |
| | SGPGTSTEPSEGSAPGSPAGSGGGSMSELLERILSNDNMNAAYKRVCANKGAGGVDGV | |
| | TVEELGDYIKENWSSIREQIRRRQYKPQPVRRVEIPKPNGGVRNLGIPTVMDRVIQQG | |
| | IVQVLSPMCEPLFSERSYGFRPNRSCEMAIRQLLVYLNEGCEWIVDIDLEKFFDNVPQ | |
| | DRLMSLVHDIINDGDTESLIRKYLKAGVMTPQGYEETKLGTPQGGNLSPLLSNIMLNE | |
| | LDKELEKRGLRFTRYADDCVIAVKSESSAKRVMRTVADWIQRKLGLKVNMTKTHITRP | |
| | TKLKYLGFGFYRDNKAKEWKCRPHQDSVAKFKRKLKELTCRKTPGTVTGRIAKINQVT | |
| | RGWINYYAIGSMKTTMIEIDAHLRTRLRVIIWKQWKVPKKRQWGLQKLGIGKDLARLT | |
| | SYYGDRYQWIVTKTCVARAISKEVLAKAGLVSCLDYYNERHALKLC | |
| 2691335- | DKKYSIGLDIGTNSVGWAVITDEYKVPSKKFKVLGNTDRHSIKKNLIGALLFDSGETA | 540 |
| RDDP | EATRLKRTARRRYTRRKNRICYLQEIFSNEMAKVDDSFFHRLEESFLVEEDKKHERHP | |
| fusion at | IFGNIVDEVAYHEKYPTIYHLRKKLVDSTDKADLRLIYLALAHMIKFRGHFLIEGDLN | |
| C term | PDNSDVDKLFIQLVQTYNQLFEENPINASGVDAKAILSARLSKSRRLENLIAQLPGEK | |
| | KNGLFGNLIALSLGLTPNFKSNFDLAEDAKLQLSKDTYDDDLDNLLAQIGDQYADLFL | |
| | AAKNLSDAILLSDILRVNTEITKAPLSASMIKRYDEHHQDLTLLKALVRQQLPEKYKE | |
| | IFFDQSKNGYAGYIDGGASQEEFYKFIKPILEKMDGTEELLVKLNREDLLRKQRTEDN | |
| | GSIPHQIHLGELHAILRRQEDFYPFLKDNREKIEKILTFRIPYYVGPLARGNSRFAWM | |
| | TRKSEETITPWNFEEVVDKGASAQSFIERMTNFDKNLPNEKVLPKHSLLYEYFTVYNE | |
| | LTKVKYVTEGMRKPAFLSGEQKKAIVDLLFKTNRKVTVKQLKEDYFKKIECFDSVEIS | |
| | GVEDRFNASLGTYHDLLKIIKDKDFLDNEENEDILEDIVLTLTLFEDREMIEERLKTY | |
| | AHLFDDKVMKQLKRRRYTGWGRLSRKLINGIRDKQSGKTILDFLKSDGFANRNFMQLI | |
| | HDDSLTFKEDIQKAQVSGQGDSLHEHIANLAGSPAIKKGILQTVKVVDELVKVMGRHK | |
| | PENIVIEMARENQTTQKGQKNSRERMKRIEEGIKELGSQILKEHPVENTQLQNEKLYL | |
| | YYLQNGRDMYVDQELDINRLSDYDVDAIVPQSFLKDDSIDNKVLTRSDKNRGKSDNVP | |
| | SEEVVKKMKNYWRQLLNAKLITQRKFDNLTKAERGGLSELDKAGFIKRQLVETRQITK | |
| | HVAQILDSRMNTKYDENDKLIREVKVITLKSKLVSDFRKDFQFYKVREINNYHHAHDA | |
| | YLNAVVGTALIKKYPKLESEFVYGDYKVYDVRKMIAKSEQEIGKATAKYFFYSNIMNE | |
| | FKTEITLANGEIRKRPLIETNGETGEIVWDKGRDFATVRKVLSMPQVNIVKKTEVQTG | |
| | GFSKESILPKRNSDKLIARKKDWDPKKYGGFDSPTVAYSVLVVAKVEKGKSKKLKSVK | |
| | ELLGITIMERSSFEKNPIDFLEAKGYKEVKKDLIIKLPKYSLFELENGRKRMLASAGE | |
| | LQKGNELALPSKYVNFLYLASHYEKLKGSPEDNEQKQLFVEQHKHYLDEIIEQISEFS | |
| | KRVILADANLDKVLSAYNKHRDKPIREQAENIIHLFTLTNLGAPAAFKYFDTTIDRKR | |
| | YTSTKEVLDATLIHQSITGLYETRIDLSQLGGDGSSGGSPAGSPTSTEEGTSESATPE | |
| | SGPGTSTEPSEGSAPGSPAGSGGGSMLDRDNLRLAYKRVVRNGGAPGVDGVTVAALQS | |
| | YLNTHWDRVKNELLAGTYRPAPVRRVEIPKPGGGVRLLGIPTVMDRFLQQALLQVMNP | |
| | IFDAHFSWYSYGFRPGKRAHDAVMQAQRYIRDGYRWVVDLDLEKFFDRVNHDMLMARV | |
| | ARKVKDKRVLKLIRAYLNAGVMINGVCQRTEEGTPQGGPLSPLLANILLDDLDKELTR | |
| | RGLRFVRYADDCNIFVASKRAGERVMESAIRFLEGKLKLKVNRDKSAVDRPWKRKFLG | |
| | FSFLSDKEATIRLAPKTISRFKERVREITSRSRPIPMEERIRRLNQYTMGWVSYERLA | |
| | SMKNHCERFDQWIRRRLRMCLWKQWKRVRTRIRELRALGVPDWACFAMANSRRGPWEM | |
| | SRNINNALPTSYWEAKGLKSMLTRYMVLRQPFGTAWCGPACQVV | |
| 2691339- | DKKYSIGLDIGTNSVGWAVITDEYKVPSKKFKVLGNTDRHSIKKNLIGALLFDSGETA | 541 |
| RDDP | EATRLKRTARRRYTRRKNRICYLQEIFSNEMAKVDDSFFHRLEESFLVEEDKKHERHP | |
| fusion at | IFGNIVDEVAYHEKYPTIYHLRKKLVDSTDKADLRLIYLALAHMIKFRGHFLIEGDLN | |
| C term | PDNSDVDKLFIQLVQTYNQLFEENPINASGVDAKAILSARLSKSRRLENLIAQLPGEK | |
| | KNGLFGNLIALSLGLTPNFKSNFDLAEDAKLQLSKDTYDDDLDNLLAQIGDQYADLFL | |
| | AAKNLSDAILLSDILRVNTEITKAPLSASMIKRYDEHHQDLTLLKALVRQQLPEKYKE | |
| | IFFDQSKNGYAGYIDGGASQEEFYKFIKPILEKMDGTEELLVKLNREDLLRKQRTEDN | |
| | GSIPHQIHLGELHAILRRQEDFYPFLKDNREKIEKILTFRIPYYVGPLARGNSRFAWM | |
| | TRKSEETITPWNFEEVVDKGASAQSFIERMTNFDKNLPNEKVLPKHSLLYEYFTVYNE | |
| | LTKVKYVTEGMRKPAFLSGEQKKAIVDLLFKTNRKVTVKQLKEDYFKKIECFDSVEIS | |
| | GVEDRFNASLGTYHDLLKIIKDKDFLDNEENEDILEDIVLTLTLFEDREMIEERLKTY | |
| | AHLFDDKVMKQLKRRRYTGWGRLSRKLINGIRDKQSGKTILDFLKSDGFANRNFMQLI | |
| | HDDSLTFKEDIQKAQVSGQGDSLHEHIANLAGSPAIKKGILQTVKVVDELVKVMGRHK | |
| | PENIVIEMARENQTTQKGQKNSRERMKRIEEGIKELGSQILKEHPVENTQLQNEKLYL | |
| | YYLQNGRDMYVDQELDINRLSDYDVDAIVPQSFLKDDSIDNKVLTRSDKNRGKSDNVP | |
| | SEEVVKKMKNYWRQLLNAKLITQRKFDNLTKAERGGLSELDKAGFIKRQLVETRQITK | |
| | HVAQILDSRMNTKYDENDKLIREVKVITLKSKLVSDFRKDFQFYKVREINNYHHAHDA | |
| | YLNAVVGTALIKKYPKLESEFVYGDYKVYDVRKMIAKSEQEIGKATAKYFFYSNIMNE | |
| | FKTEITLANGEIRKRPLIETNGETGEIVWDKGRDFATVRKVLSMPQVNIVKKTEVQTG | |
| | GFSKESILPKRNSDKLIARKKDWDPKKYGGFDSPTVAYSVLVVAKVEKGKSKKLKSVK | |
| | ELLGITIMERSSFEKNPIDFLEAKGYKEVKKDLIIKLPKYSLFELENGRKRMLASAGE | |
| | LQKGNELALPSKYVNFLYLASHYEKLKGSPEDNEQKQLFVEQHKHYLDEIIEQISEFS | |
| | KRVILADANLDKVLSAYNKHRDKPIREQAENIIHLFTLTNLGAPAAFKYFDTTIDRKR | |
| | YTSTKEVLDATLIHQSITGLYETRIDLSQLGGDGSSGGSPAGSPTSTEEGTSESATPE | |
| | SGPGTSTEPSEGSAPGSPAGSGGGSENPIKIDFFEPLEIEPLNLIDVNFVEKMDMDID | |
| | LSYEQDSLLKQNLKNLRLDHCNKEEKDAIRKLCFDYRDIFYCEQIPLSFTSEITHKIK | |
| | LNDESPIYTKSYRFPEIHKKEVKDQIAKMLDQGIIQHSISPWSSPIWVVPKKLDASGR | |
| | RKWRLVIDYRKLNEKSCNDRYPLPNITDILDKLGRANYFTTLDLASGFHQIQVDPDDI | |
| | PKTAFSTEGGHYEFKRMPFGLKNAPSTFQRVMDNILRGLHNEICLVYLDDIIIFSTSL | |
| | DEHIQRLKSVFDRLRKSNFKLQLDKSEFLQKTVQYLGHIITPQGVKPNPDKVSTIKRF | |
| | PIPRTQKDIKSFLGLLGYYRRFIKDFAKITKPLTKCLKKNAKVTHDQNFIDAFNTCKE | |
| | ILVNDPILQYPDFSKPFILTTDASDVALGAILSQGTLPNDRPVAYASRTLNETESKYS | |
| | TIEKELLAIVWACKHERPYLYGRKFTIYTDHRPLTWLFSLKEPNSKLVRWRLKLEEFE | |
| | YDIIYKKGKLNTNADCLSRVTLNAIDNESMLNNPGDIDQDILDTLQNVTQNLQTLQDE | |
| | QKPSTSNTNDKINIISDIQIKPPDNSQNDNTNTSTQHSTANETINDGIKIFDEIINNK | |
| | TNQILVFPNVIYKLDIKRETYENHKIVTVKIPIINNEQMILQFLKENTDPKHVYCMYF | |
| | QSDELYNDFCTVYLKHFSEKGPKLIRCLKLVNTVADKEEQILLIKNHHGSKINHRGIN | |
| | ETLEKLKFNYYWKNMKSTVSNFINACDICQRA | |
| 2691355- | DKKYSIGLDIGTNSVGWAVITDEYKVPSKKFKVLGNTDRHSIKKNLIGALLFDSGETA | 542 |
| RDDP | EATRLKRTARRRYTRRKNRICYLQEIFSNEMAKVDDSFFHRLEESFLVEEDKKHERHP | |
| fusion at | IFGNIVDEVAYHEKYPTIYHLRKKLVDSTDKADLRLIYLALAHMIKFRGHFLIEGDLN | |
| C term | PDNSDVDKLFIQLVQTYNQLFEENPINASGVDAKAILSARLSKSRRLENLIAQLPGEK | |
| | KNGLFGNLIALSLGLTPNFKSNFDLAEDAKLQLSKDTYDDDLDNLLAQIGDQYADLFL | |
| | AAKNLSDAILLSDILRVNTEITKAPLSASMIKRYDEHHQDLTLLKALVRQQLPEKYKE | |
| | IFFDQSKNGYAGYIDGGASQEEFYKFIKPILEKMDGTEELLVKLNREDLLRKQRTEDN | |
| | GSIPHQIHLGELHAILRRQEDFYPFLKDNREKIEKILTFRIPYYVGPLARGNSRFAWM | |
| | TRKSEETITPWNFEEVVDKGASAQSFIERMTNFDKNLPNEKVLPKHSLLYEYFTVYNE | |
| | LTKVKYVTEGMRKPAFLSGEQKKAIVDLLFKTNRKVTVKQLKEDYFKKIECFDSVEIS | |
| | GVEDRFNASLGTYHDLLKIIKDKDELDNEENEDILEDIVLTLTLFEDREMIEERLKTY | |
| | AHLFDDKVMKQLKRRRYTGWGRLSRKLINGIRDKQSGKTILDFLKSDGFANRNFMQLI | |
| | HDDSLTFKEDIQKAQVSGQGDSLHEHIANLAGSPAIKKGILQTVKVVDELVKVMGRHK | |
| | PENIVIEMARENQTTQKGQKNSRERMKRIEEGIKELGSQILKEHPVENTQLQNEKLYL | |
| | YYLQNGRDMYVDQELDINRLSDYDVDAIVPQSFLKDDSIDNKVLTRSDKNRGKSDNVP | |
| | SEEVVKKMKNYWRQLLNAKLITQRKEDNLTKAERGGLSELDKAGFIKRQLVETRQITK | |
| | HVAQILDSRMNTKYDENDKLIREVKVITLKSKLVSDFRKDFQFYKVREINNYHHAHDA | |
| | YLNAVVGTALIKKYPKLESEFVYGDYKVYDVRKMIAKSEQEIGKATAKYFFYSNIMNE | |
| | FKTEITLANGEIRKRPLIETNGETGEIVWDKGRDFATVRKVLSMPQVNIVKKTEVQTG | |
| | GFSKESILPKRNSDKLIARKKDWDPKKYGGFDSPTVAYSVLVVAKVEKGKSKKLKSVK | |
| | ELLGITIMERSSFEKNPIDFLEAKGYKEVKKDLIIKLPKYSLFELENGRKRMLASAGE | |
| | LQKGNELALPSKYVNFLYLASHYEKLKGSPEDNEQKQLFVEQHKHYLDEIIEQISEFS | |
| | KRVILADANLDKVLSAYNKHRDKPIREQAENIIHLFTLTNLGAPAAFKYFDTTIDRKR | |
| | YTSTKEVLDATLIHQSITGLYETRIDLSQLGGDGSSGGSPAGSPTSTEEGTSESATPE | |
| | SGPGTSTEPSEGSAPGSPAGSGGGSVLIRDTSITTNPLPARYHDFSDVFEKKNADRLP | |
| | EHRLYDCPIDLQEGAHPPFGPIYGLAEPELEALREYLKENLAKGFIQPSKSPAGAPIL | |
| | FVKKKDGSLRLCVDYRGLNRVTVRNRYPLPLIPELLDRLRTGRVFSKIDLRGAYNLVR | |
| | IKPGDEWKTAFRTRYGHFEYKVMPFGLTNAPAVFQHMMNHIFREYLDHFVVIYLDDIL | |
| | VFSPSMEEHTRHVRLILTKLREHGLYAKSEKCEFDRTSVEFLGYVISPAGITMDPRKV | |
| | ATIHNWPIPTRLKEVQSFLGFANFYRRFIDRESTIVQPLISLTRKDVPFVWTATTQHA | |
| | FDALKQAFMSAPVLVHPNPAKPFQVETDASDFALGAILSQLDDDGTLHPVAYYSRKFL | |
| | ASEINYPVYDKELAAIISAFAEWRPYLVGAQHRIQVVTDHKNLLYFASSRTLNRRQAR | |
| | WSIFLADYDFEIIFRPGIQHGKADALSRRPDLALCPGDDAYTQQSCSLLKPDQLQLSA | |
| | TFMLHDDSLLQEIAQATKTDTFATEILKRLQDSSPATKRADLHHFTTHDGLLYRNHLL | |
| | YVPEGPCRTQVLQTCHDDPLAGHFGVAKTLELVSRGFWWPQPWKLVKEFVKTCDVCAR | |
| | SKAVHHRPYGLLHPLPIPNRPWASISMDFITDLPPVNGVDTV | |

### Example 9—Novel RNA-Dependent DNA Polymerases for Precision Editing

Additional RDDPs were identified according to the methods provided in Example 8 (SEQ ID: 545-567 in Table A). On average, each of these newly identified RDDPs was approximately 30% smaller than the MMLV-RT (See Table 7). Each of the RDDP candidates was fused to nCas9 (H840A) (SEQ ID NO: 544) on the N terminus or the C terminus, separated by an XTEN40 linker (SEQ ID NO: 543). Each fusion protein was tested against two targets in HEK293T cells for precision editing: (1) HEK site 3 for an A insertion at the +1 position relative to the non-target strand cut site and (2) FANCF for a G to T substitution at the +5 position. Two control RDDPs were included: the wild-type Moloney murine leukemia virus (M-MLV) RDDP (SEQ ID NO: 531) and the evolved M-MLV RDDP (SEQ ID NO: 532). Each composition comprising the polynucleotide encoding one of the fusion proteins was transfected into HEK293T cells. 3 days post-transfection, the precision editing outcomes were assessed by next generation sequence (NGS). Each test had two independent replicates.

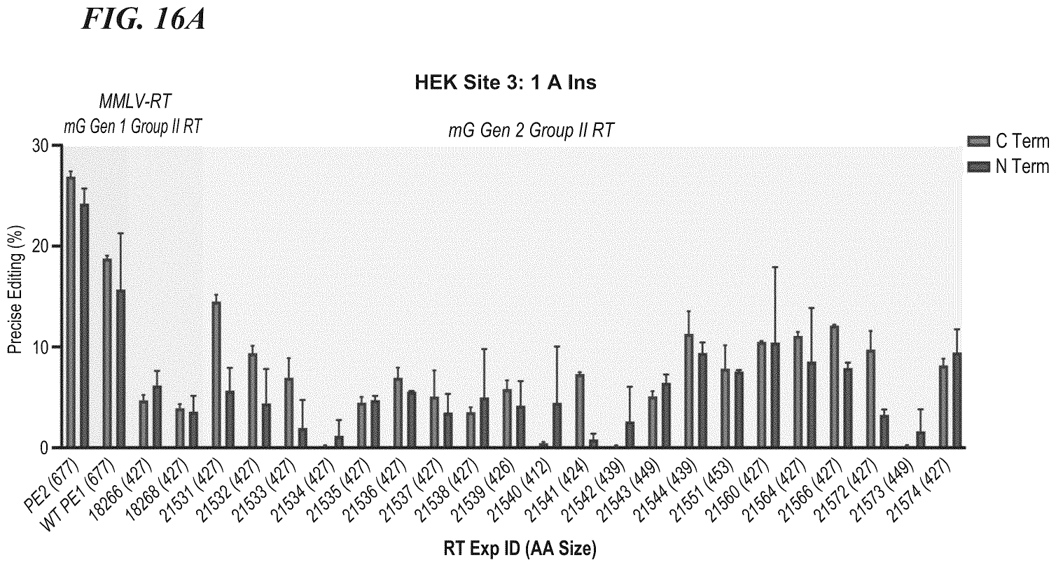

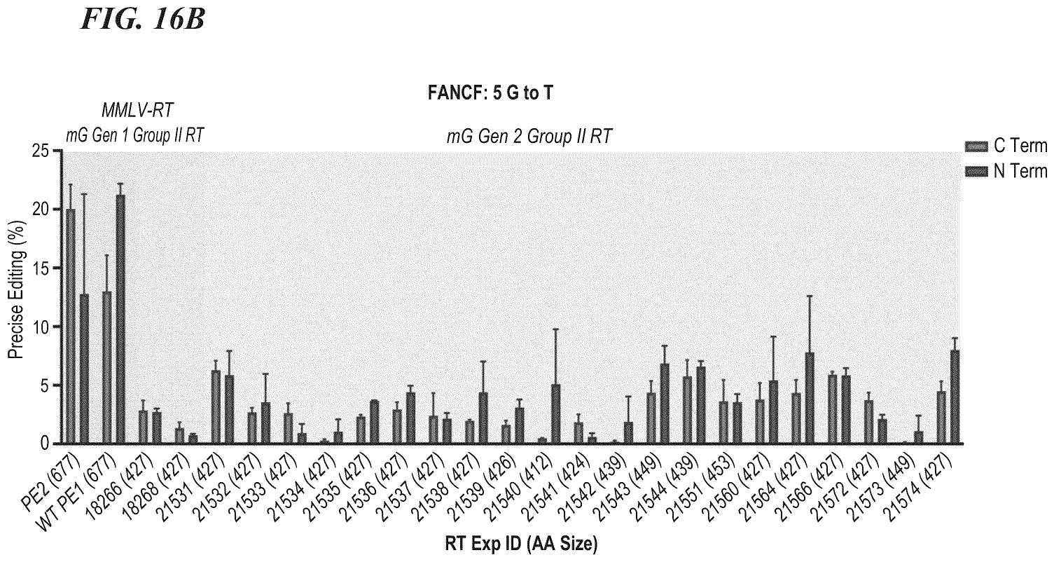

The results of this experiment are shown in FIG. 16A (HEK) and FIG. 16B (FANCF). As shown, up to 14% precise editing was achieved (See 21531).

### Example 10—Variant RNA-Dependent DNA Polymerases for Precision Editing

Variants of the 2691319 RDDP (SEQ ID NO: 496) and 2619323 RDDP (SEQ ID NO: 498) were generated and tested for their ability to perform precision editing. Combinations of arginine and lysine mutations were made at positions D12, N24, D72, N195, and N114 as follows:
(a) 2691319 (D12R-D72R-N195R)—SEQ ID NO: 568
(b) 2691319 (D12R-N24R-D72R-N114K)—SEQ ID NO: 569
(c) 2691319 (D12R-N24R-D72R-N195R)—SEQ ID NO: 570
(d) 2691319 (D12R-D72R-N114K-N195R)—SEQ ID NO: 571
(e) 2691319 (D12R-N24R-D72R-N114K-N195R)—SEQ ID NO: 572
(f) 2691323 (D12R-N72R-N195R)—SEQ ID NO: 573
(g) 2691323 (D12R-N24R-N72R-N114K)—SEQ ID NO: 574
(h) 2691323 (D12R-N24R-N72R-N195R)—SEQ ID NO: 575
(i) 2691323 (D12R-N72R-N114K-N195R)—SEQ ID NO: 576
(j) 2691323 (D12R-N24R-N72R-N114K-N195R)—SEQ ID NO: 577.

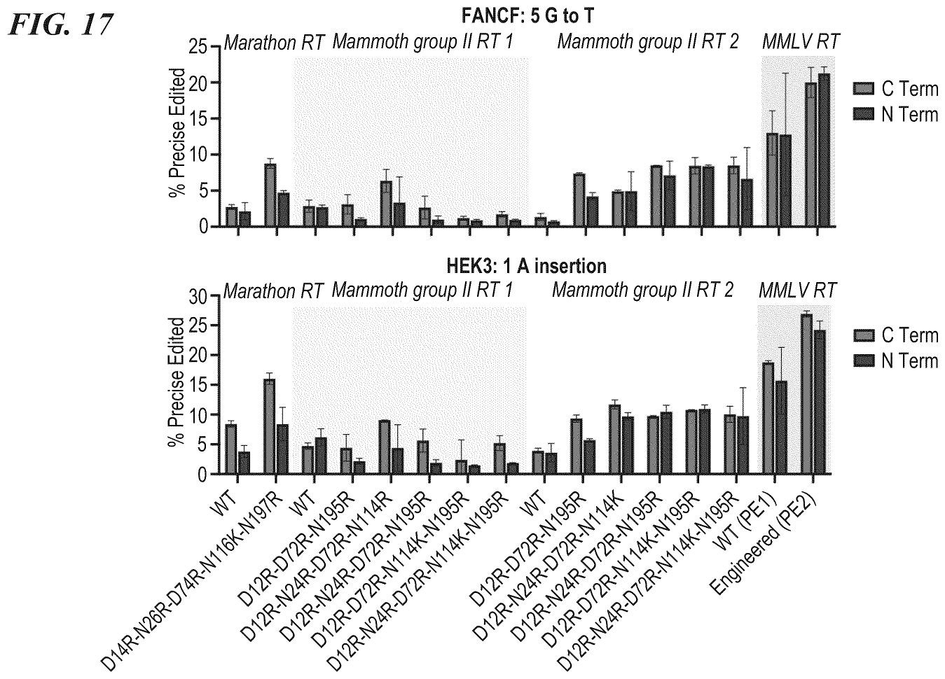

Each of the engineered RDDP candidates was fused to nCas9 (H840A) (SEQ ID NO: 544) on the N terminus or the C terminus, separated by an XTEN40 linker (SEQ ID NO: 543). The results are shown in FIG. 17. The above-described modifications increased editing by up to 4-fold compared to the editing observed with the parental RDDPs, increasing precision editing from ˜2.4% to ˜9.6%.

1. A system comprising:
a) an effector protein or a nucleic acid encoding the effector protein;
b) an RNA-directed DNA polymerase (RDDP) or a nucleic acid encoding the RDDP;
c) a guide RNA or nucleic acid encoding the guide RNA, wherein the guide RNA comprises
i. a first region comprising a protein binding sequence, and
ii. a second region comprising a spacer sequence that hybridizes to a target sequence of a first strand of a double stranded DNA (dsDNA) target nucleic acid,
wherein the first region is located 5′ of the second region; and
d) a template RNA (rttRNA) or nucleic acid encoding the rttRNA, wherein the rttRNA comprises
i. a primer binding sequence (PBS), and
ii. a template sequence that hybridizes to the target sequence of a second strand of the dsDNA target nucleic acid.

2. The system of claim 1, wherein the guide RNA is linked to the rttRNA.

3. The system of claim 1 or 2, wherein the template sequence is located 5′ of the PBS, optionally wherein the 3′ end of the PBS is linked to the 5′ end of the template sequence.

4. The system of any one of claims 1-3, wherein the rttRNA is circularized.

5. The system of any one of claims 1-4, wherein the rttRNA comprises a protein localization sequence that can localize a protein to the rttRNA.

6. The system of claim 5, wherein the protein localization sequence comprises an MS2 coat protein localization sequence.

7. The system of claim 6, wherein the RDDP is not fused to the effector protein, optionally wherein the RDDP is fused to an MS2 coat protein.

8. The system of any one of claims 1-4, wherein the RDDP is fused to the effector protein.

9. The system of any one of claims 1-8, wherein the template sequence comprises a difference of at least one nucleotide relative to an equal length portion of the target sequence.

10. The system of any one of claims 1-9, wherein the effector protein is a Type V Cas protein.

11. The system of any one of claims 1-10, wherein the length of the effector protein is 400 to 800 linked amino acids.

12. The system of any one of claims 1-11, wherein the effector protein comprises an amino acid sequence that is at least 65%, at least 70%, at least 75%, at least 80%, at least 85%, at least 90%, at least 95%, at least 97%, at least 98%, at least 99%, or 100% identical to a sequence in TABLE 1.

13. The system of any one of claims 1-11, wherein the effector protein comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 12.

14. The system of any one of claims 1-11, wherein the effector protein comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 346.

15. The system of claim 14, wherein the target nucleic acid comprises a PAM sequence and at least a portion of the PBS is complementary at least a portion of the target nucleic acid sequence that is 5′ of the nucleotide at position 13 relative to the PAM sequence.

16. The system of any one of claims 1-15, wherein the effector protein comprises at least one amino acid alteration relative to a relative amino acid sequence in TABLE 1 that results in reduced nuclease activity, nickase activity, increased nickase activity, or a combination thereof.

17. The system of any one of claims 1-15, wherein the effector protein is a nickase, or wherein the method is performed under conditions wherein the effector protein has nickase activity.

18. The system of any one of claims 1-17, wherein the primer binding sequence is less than 20, less than 19, less than 18, less than 17, less than 16, less than 16, less than 15, less than 14, less than 13, less than 12, less than 11, less than 10, less than 9, less than 8, less than 7, less than 6, less than 5 nucleotides long, and at least 4 nucleotides long.

19. The system of any one of claims 1-18, wherein the template sequence is less than 35, less than 34, less than 33, less than 32, less than 31, less than 30, less than 29, less than 28, less than 27, less than 26, less than 25, less than 24, less than 23, less than 22, less than 21, less than 20, less than 19, less than 18 nucleotides long, and at least 8 nucleotides long.

20. The system of any one of claims 1-19, wherein the RDDP comprises an amino acid sequence that is at least 65%, at least 70%, at least 75%, at least 80%, at least 85%, at least 90%, at least 95%, at least 97%, at least 98%, at least 99%, or 100% identical to any one of SEQ ID NOs: 485-530, 545-567, or 568-577.

21. The system of any one of claims 1-19, wherein the RDDP comprises an amino acid sequence that is at least 65%, at least 70%, at least 75%, at least 80%, at least 85%, at least 90%, at least 95%, at least 97%, at least 98%, at least 99%, or 100% identical to any one of SEQ ID NOs: 496, 498, 504, 506, or 514.

22. The system of any one of claims 1-21, comprising an expression vector, wherein the expression vector comprises any combination of: the nucleic acid encoding the effector protein; the nucleic acid encoding the RDDP; the nucleic acid encoding the guide RNA; and the nucleic acid encoding the rttRNA.

23. The system of claim 22, wherein the expression vector is an adeno-associated viral (AAV) vector, optionally wherein the AAV vector is an scAAV vector.

24. The system of any one of claims 1-23, comprising a lipid or lipid nanoparticle.

25. The system of any one of claims 1-24, wherein the nucleic acid encoding the effector protein or the nucleic acid encoding the RDDP comprises a messenger RNA.

26. The system of any one of claims 1-25, comprising a non-homologous end joining (NHEJ) inhibitor.

27. A pharmaceutical composition comprising any one of the effector protein, RDDP, guide RNA, template RNA, and a nucleic acid encoding the same; and a pharmaceutically acceptable excipient.

28. A cell modified by the system of any one of claims 1-26.

29. A cell comprising the system of any one of claims 1-26.

30. The cell of claim 28 or 29, wherein the cell is a eukaryotic cell.

31. A method of modifying a target nucleic acid, the method comprising contacting a target nucleic acid with the system of any one of claims 1-26.

32. The method of claim 31, wherein the cell is a eukaryotic cell.


SEQUENCE LISTING (581 sequences)

SEQ ID NO: 1 - protein, 707 aa, unidentified
MADTPTLFTQFLRHHLPGQRFRKDILKQAGRILANKGEDATIAFLRGKSEESPPDFQPPVKCPIIACSRPLTEWPIYQASVAIQGYVYGQSLAEFEASDPGCSKDGLLGWFDKTGVCTDYFSVQGLNLIFQNARKRYIGVQTKVTNRNEKRHKKLKRINAKRIAEGLPELTSDEPESALDETGHLIDPPGLNTNIYCYQQVSPKPLALSEVNQLPTAYAGYSTSGDDPIQPMVTKDRLSISKGQPGYIPEHQRALLSQKKHRRMRGYGLKARALLVIVRIQDDWAVIDLRSLLRNAYWRRIVQTKEPSTITKLLKLVTGDPVLDATRMVATFTYKPGIVQVRSAKCLKNKQGSKLFSERYLNETVSVTSIDLGSNNLVAVATYRLVNGNTPELLQRFTLPSHLVKDFERYKQAHDTLEDSIQKTAVASLPQGQQTEIRMWSMYGFREAQERVCQELGLADGSIPWNVMTATSTILTDLFLARGGDPKKCMFTSEPKKKKNSKQVLYKIRDRAWAKMYRTLLSKETREAWNKALWGLKRGSPDYARLSKRKEELARRCVNYTISTAEKRAQCGRTIVALEDLNIGFFHGRGKQEPGWVGLFTRKKENRWLMQALHKAFLELAHHRGYHVIEVNPAYTSQTCPVCRHCDPDNRDQHNREAFHCIGCGFRGNADLDVATHNIAMVAITGESLKRARGSVASKTPQPLAAE

SEQ ID NO: 2 - protein, 757 aa, unidentified
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 3 - protein, 765 aa, unidentified
MYILEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 4 - protein, 766 aa, unidentified
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKAFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 5 - protein, 812 aa, unidentified
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 6 - protein, 812 aa, unidentified
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVDIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHKGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 7 - protein, 793 aa, unidentified
MSSLPTPLELLKQKHADLFKGLQFSSKDNKMAGKVLKKDGEEAALAFLSERGVSRGELPNFRPPAKTLVVAQSRPFEEFPIYRVSEAIQLYVYSLSVKELETVPSGSSTKKEHQRFFQDSSVPDFGYTSVQGLNKIFGLARGIYLGVITRGENQLQKAKSKHEALNKKRRASGEAETEFDPTPYEYMTPERKLAKPPGVNHSIMCYVDISVDEFDFRNPDGIVLPSEYAGYCREINTAIEKGTVDRLGHLKGGPGYIPGHQRKESTTEGPKINFRKGRIRRSYTALYAKRDSRRVRQGKLALPSYRHHMMRLNSNAESAILAVIFFGKDWVVFDLRGLLRNVRWRNLFVDGSTPSTLLGMFGDPVIDPKRGVVAFCYKEQIVPVVSKSITKMVKAPELLNKLYLKSEDPLVLVAIDLGQTNPVGVGVYRVMNASLDYEVVTRFALESELLREIESYRQRTNAFEAQIRAETFDAMTSEEQEEITRVRAFSASKAKENVCHRFGMPVDAVDWATMGSNTIHIAKWVMRHGDPSLVEVLEYRKDNEIKLDKNGVPKKVKLTDKRIANLTSIRLRFSQETSKHYNDTMWELRRKHPVYQKLSKSKADFSRRVVNSIIRRVNHLVPRARIVFIIEDLKNLGKVFHGSGKRELGWDSYFEPKSENRWFIQVLHKAFSETGKHKGYYIIECWPNWTSCTCPKCSCCDSENRHGEVFRCLACGYTCNTDFGTAPDNLVKIATTGKGLPGPKKRCKGSSKGKNPKIARSSETGVSVTESGAPKVKKSSPTQTSQSSSQSAP

SEQ ID NO: 8 - protein, 441 aa, unidentified
MNKIEKEKTPLAKLMNENFAGLRFPFAIIKQAGKKLLKEGELKTIEYMTGKGSIEPLPNFKPPVKCLIVAKRRDLKYFPICKASCEIQSYVYSLNYKDFMDYFSTPMTSQKQHEEFFKKSGLNIEYQNVAGLNLIFNNVKNTYNGVILKVKNRNEKLKKKAIKNNYEFEEIKTFNDDGCLINKPGINNVIYCFQSISPKILKNITHLPKEYNDYDCSVDRNIIQKYVSRLDIPESQPGHVPEWQRKLPEFNNTNNPRRRRKWYSNGRNISKGYSVDQVNQAKIEDSLLAQIKIGEDWIILDIRGLLRDLNRRELISYKNKLTIKDVLGFFSDYPIIDIKKNLVTFCYKEGVIQVVSQKSIGNKKSKQLLEKLIENKPIALVSIDLGQTNPVSVKISKLNKINNKISIESFTYRFLNEEILKEIEKYRKDYDKLELKLINEA

SEQ ID NO: 9 - protein, 812 aa, unidentified
MDMLDTETNYATETPSQQQDYSPKPPKKDRRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVDIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 10 - protein, 812 aa, unidentified
MDMLDTETNYATETPSQQQDYSPKPPKKDRRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 11 - protein, 761 aa, unidentified
MSNKTTPPSPLSLLLRAHFPGLKFESQDYKIAGKKLRDGGPEAVISYLTGKGQAKLKDVKPPAKAFVIAQSRPFIEWDLVRVSRQIQEKIFGIPATKGRPKQDGLSETAFNEAVASLEVDGKSKLNEETRAAFYEVLGLDAPSLHAQAQNALIKSAISIREGVLKKVENRNEKNLSKTKRRKEAGEEATFVEEKAHDERGYLIHPPGVNQTIPGYQAVVIKSCPSDFIGLPSGCLAKESAEALTDYLPHDRMTIPKGQPGYVPEWQHPLLNRRKNRRRRDWYSASLNKPKATCSKRSGTPNRKNSRTDQIQSGRFKGAIPVLMRFQDEWVIIDIRGLLRNARYRKLLKEKSTIPDLLSLFTGDPSIDMRQGVCTFIYKAGQACSAKMVKTKNAPEILSELTKSGPVVLVSIDLGQTNPIAAKVSRVTQLSDGQLSHETLLRELLSNDSSDGKEIARYRVASDRLRDKLANLAVERLSPEHKSEILRAKNDTPALCKARVCAALGLNPEMIAWDKMTPYTEFLATAYLEKGGDRKVATLKPKNRPEMLRRDIKFKGTEGVRIEVSPEAAEAYREAQWDLQRTSPEYLRLSTWKQELTKRILNQLRHKAAKSSQCEVVVMAFEDLNIKMMHGNGKWADGGWDAFFIKKRENRWFMQAFHKSLTELGAHKGVPTIEVTPHRTSITCTKCGHCDKANRDGERFACQKCGFVAHADLEIATDNIERVALTGKPMPKPESERSGDAKKSVGARKAAFKPEEDAEAAE

SEQ ID NO: 12 - protein, 717 aa, unidentified
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 13 - protein, 781 aa, unidentified
MRQPAEKTAFQVFRQEVIGTQKLSGGDAKTAGRLYKQGKMEAAREWLLKGARDDVPPNFQPPAKCLVVAVSHPFEEWDISKTNHDVQAYIYAQPLQAEGHLNGLSEKWEDTSADQHKLWFEKTGVPDRGLPVQAINKIAKAAVNRAFGVVRKVENRNEKRRSRDNRIAEHNRENGLTEVVREAPEVATNADGFLLHPPGIDPSILSYASVSPVPYNSSKHSFVRLPEEYQAYNVEPDAPIPQFVVEDRFAIPPGQPGYVPEWQRLKCSTNKHRRMRQWSNQDYKPKAGRRAKPLEFQAHLTRERAKGALLVVMRIKEDWVVFDVRGLLRNVEWRKVLSEEAREKLTLKGLLDLFTGDPVIDTKRGIVTFLYKAEITKILSKRTVKTKNARDLLLRLTEPGEDGLRREVGLVAVDLGQTHPIAAAIYRIGRTSAGALESTVLHRQGLREDQKEKLKEYRKRHTALDSRLRKEAFETLSVEQQKEIVTVSGSGAQITKDKVCNYLGVDPSTLPWEKMGSYTHFISDDFLRRGGDPNIVHFDRQPKKGKVSKKSQRIKRSDSQWVGRMRPRLSQETAKARMEADWAAQNENEEYKRLARSKQELARWCVNTLLQNTRCITQCDEIVVVIEDLNVKSLHGKGAREPGWDNFFTPKTENRWFIQILHKTFSELPKHRGEHVIEGCPLRTSITCPACSYCDKNSRNGEKFVCVACGATFHADFEVATYNLVRLATTGMPMPKSLERQGGGEKAGGARKARKKAKQVEKIVVQANANVTMNGASLHSP

SEQ ID NO: 14 - protein, 793 aa, unidentified; identical to SEQ ID NO: 7

SEQ ID NO: 15 - protein, 717 aa, unidentified; identical to SEQ ID NO: 12

SEQ ID NO: 16 - protein, 761 aa, unidentified; identical to SEQ ID NO: 11

SEQ ID NO: 17 - protein, 765 aa, unidentified
MYSLEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 18 - protein, 766 aa, unidentified
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKTFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 19 - protein, 870 aa, unidentified
MLVRTSTLVQDNKNSRSASRAFLKKPKMPKNKHIKEPTELAKLIRELFPGQRFTRAINTQAGKILKHKGRDEVVEFLKNKGIDKEQFMDFRPPTKARIVATSGAIEEFSYLRVSMAIQECCFGKYKFPKEKVNGKLVLETVGLTKEELDDFLPKKYYENKKSRDRFFLKTGICDYGYTYAQGLNEIFRNTRAIYEGVFTKVNNRNEKRREKKDKYNEERRSKGLSEEPYDEDESATDESGHLINPPGVNLNIWTCEGFCKGPYVTKLSGTPGYEVILPKVFDGYNRDPNEIISCGITDRFAIPEGEPGHIPWHQRLEIPEGQPGYVPGHQRFADTGQNNSGKANPNKKGRMRKYYGHGTKYTQPGEYQEVFRKGHREGNKRRYWEEDFRSEAHDCILYVIHIGDDWVVCDLRGPLRDAYRRGLVPKEGITTQELCNLFSGDPVIDPKHGVVTFCYKNGLVRAQKTISAGKKSRELLGALTSQGPIALIGVDLGQTEPVGARAFIVNQARGSLSLPTLKGSFLLTAENSSSWNVFKGEIKAYREAIDDLAIRLKKEAVATLSVEQQTEIESYEAFSAEDAKQLACEKFGVDSSFILWEDMTPYHTGPATYYFAKQFLKKNGGNKSLIEYIPYQKKKSKKTPKAVLRSDYNIACCVRPKLLPETRKALNEAIRIVQKNSDEYQRLSKRKLEFCRRVVNYLVRKAKKLTGLERVIIAIEDLKSLEKFFTGSGKRDNGWSNFFRPKKENRWFIPAFHKAFSELAPNRGFYVIECNPARTSITDPDCGYCDGDNRDGIKFECKKCGAKHHTDLDVAPLNIAIVAVTGRPMPKTVSNKSKRERSGGEKSVGASRKRNHRKSKANQEMLDATSSAAE

SEQ ID NO: 20 - protein, 830 aa, unidentified
MPKIKKPTEISLLRKEVFPDLHFAKDRMRAASLVLKNEGREAAIEYLRVNHEDKPPNFMPPAKTPYVALSRPLEQWPIAQASIAIQKYIFGLTKDEFSATKKLLYGDKSTPNTESRKRWFEVTGVPNFGYMSAQGLNAIFSGALARYEGVVQKVENRNKKRFEKLSEKNQLLIEEGQPVKDYVPDTAYHTPETLQKLAENNHVRVEDLGDMIDRLVHPPGIHRSIYGYQQVPPFAYDPDNPKGIILPKAYAGYTRKPHDIIEAMPNRLNIPEGQAGYIPEHQRDKLKKGGRVKRLRTTRVRVDATETVRAKAEALNAEKARLRGKEAILAVFQIEEDWALIDMRGLLRNVYMRKLIAAGELTPTTLLGYFTETLTLDPRRTEATFCYHLRSEGALHAEYVRHGKNTRELLLDLTKDNEKIALVTIDLGQRNPLAAAIFRVGRDASGDLTENSLEPVSRMLLPQAYLDQIKAYRDAYDSFRQNIWDTALASLTPEQQRQILAYEAYTPDDSKENVLRLLLGGNVMPDDLPWEDMTKNTHYISDRYLADGGDPSKVWFVPGPRKRKKNAPPLKKPPKPRELVKRSDHNISHLSEFRPQLLKETRDAFEKAKIDTERGHVGYQKLSTRKDQLCKEILNWLEAEAVRLTRCKTMVLGLEDLNGPFFNQGKGKVRGWVSFFRQKQENRWIVNGFRKNALARAHDKGKYILELWPSWTSQTCPKCKHVHADNRHGDDFVCLQCGARLHADAEVATWNLAVVAIQGHSLPGPVREKSNDRKKSGSARKSKKANESGKVVGAWAAQATPKRATSKKETGTARNPVYNPLETQASCPAP

SEQ ID NO: 21 - protein, 790 aa, unidentified
MTPSPQIARLVETPLAAALKAHHPGKKFRSDYLKKAGKILKDQGVEAAMAHLDGKDQAEPPNFKPPAKCRIVARSREFSEWPIVKASVEIQKYIYGLTLEERKACDPGKSSASHKAWFAKTGVNTFGYSSVQGFNLIFGHTLGRYDGVLVKTENLNKKRAEKNERFRAKALAEGRAEPVCPPLVTATNDTGQDVTLEDGRVVRPGQLLQPPGINPNIYAYQQVSPKAYVPGIIELPEEFQGYSRDPNAVILPLVPRDRLSIPKGQPGYVPEPHREGLTGRKDRRMRRYYETERGTKLKRPPLTAKGRADKANEALLVVVRIDSDWVVMDVRGLLRNARWRRLVSKEGITLNGLLDLFTGDPVLNPKDCSVSRDTGDPVNDPRHGVVTFCYKLGVVDVCSKDRPIKGFRTKEVLERLTSSGTVGMVSIDLGQTNPVAAAVSRVTKGLQAETLETFTLPDDLLGKVRAYRAKTDRMEEGFRRNALRKLTAEQQAEITRYNDATEQQAKALVCSTYGIGPEEVPWERMTSNTTYISDHILDHGGDPDTVFFMATKRGQNKPTLHKRKDKAWGQKFRPAISVETRLARQAAEWELRRASLEFQKLSVWKTELCRQAVNYVMERTKKRTQCDVIIPVIEDLPVPLFHGSGKRDPGWANFFVHKRENRWFIDGLHKAFSELGKHRGIYVFEVCPQRTSITCPKCGHCDPDNRDGEKFVCLSCQATLNADLDVATTNLVRVALTGKVMPRSERSGDAQTPGPARKARTGKIKGSKPTSAPQGATQTDAKAHLSQTGV

SEQ ID NO: 22 - protein, 790 aa, unidentified
MTPSPQIARLVETPLAAALKAHHPGKKFRSDYLKKAGKILKDQGVEAAMAHLDGKDQAEPPNFKPPAKCRIVARSREFSEWPIVKASVEIQKYIYGLTLEERKACDPGKSSASHKAWFAKTGVNTFGYSSVQGFNLIFGHTLGRYDGVLVKTENLNKKRAEKNERFRAKALAEGRAEPVCPPLVTATNDTGQDVTLEDGRVVRPGQLLQPPGINPNIYAYQQVSPKAYVPGIIELPEEFQGYSRDPNAVILPLVPRDRLSIPKGQPGYVPEPHREGLTGRKDRRMRRYYETERGTKLKRPPLTAKGRADKANEALLVVVRIDSDWVVMDVRGLLRNARWRRLVSKEGITLNGLLDLFTGDPVLNPKDCSVSRDTGDPVNDPRHGVVTFCYKLGVVDVCSKDRPIKGFRTKEVLERLTSSGTVGMVSIDLGQTNPVAAAVSRVTKGLQAETLETFTLPDDLLGKVRAYRAKTDRMEEGFRRNALRKLTAEQQAEITRYNDATEQQAKALVCSTYGIGPEEVPWERMTSNTTYISDHILDHGGDPDTVFFMATKRGQNKPTLHKRKDKAWGQKFRPAISVETRLARQAAEWELRRASLEFQKLSVWKTELCRQAVNYVMERTKKRTQCDVIIPVIEDLPVPLFHGSGKRDPGWANFFVHKRENRWFIDGLHKAFSELGKHRGIYVFEVCPQRTSITCPKCGHCDPDNRDGEKFVCLSCQATLHADLDVATTNLVRVALTGKVMPRSERSGDAQTPGPARKARTGKIKGSKPTSAPQGATQTDAKAHLSQTGV

SEQ ID NO: 23 - protein, 782 aa, unidentified
MKTEKPKTALTLLREEVFPGKKYRLDVLKEAGKKLSTKGREATIEFLTGKDEERPQNFQPPAKTSIVAQSRPFDQWPIVQVSLAVQKYIYGLTQSEFEANKKALYGETGKAISTESRRAWFEATGVDNFGFTAAQGINPIFSQAVARYEGVIKKVENRNEKKLKKLTKKNLLRLESGEEIEDFEPEATFNEEGRLLQPPGANPNIYCYQQISPRIYDPSDPKGVILPQIYAGYDRKPEDIISAGVPNRLAIPEGQPGYIPEHQRAGLKTQGRIRCRASVEAKARAAILAVVHLGEDWVVLDLRGLLRNVYWRKLASPGTLTLKGLLDFFTGGPVLDARRGIATFSYTLKSAAAVHAENTYKGKGTREVLLKLTENNSVALVTVDLGQRNPLAAMIARVSRTSQGDLTYPESVEPLTRLFLPDPFLEEVRKYRSSYDALRLSIREAAIASLTPEQQAEIRYIEKFSAGDAKKNVAEVFGIDPTQLPWDAMTPRTTYISDLFLRMGGDRSRVFFEVPPKKAKKAPKKPPKKPAGPRIVKRTDGMIARLREIRPRLSAETNKAFQEARWEGERSNVAFQKLSVRRKQFARTVVNHLVQTAQKMSRCDTVVLGIEDLNVPFFHGRGKYQPGWEGFFRQKKENRWLINDMHKALSERGPHRGGYVLELTPFWTSLRCPKCGHTDSANRDGDDFVCVKCGAKLHSDLEVATANLALVAITGQSIPRPPREQSSGKKSTGTARMKKTSGETQGKGSKACVSEALNKIEQGTARDPVYNPLNSQVSCPAP

SEQ ID NO: 24 - protein, 774 aa, unidentified
VYNPDMKKPNNIRRIREEHFEGLCFGKDVLTKAGKIYEKDGEEAAIDFLMGKDEEDPPNFKPPAKTTIVAQSRPFDQWPIYQVSQAVQERVFAYTEEEFNASKEALFSGDISSKSRDFWFKTNNISDQGIGAQGLNTILSHAFSRYSGVIKKVENRNKKRLKKLSKKNQLKIEEGLEILEFKPDSAFNENGLLAQPPGINPNIYGYQAVTPFVFDPDNPGDVILPKQYEGYSRKPDDIIEKGPSRLDIPKGQPGYVPEHQRKNLKKKGRVRLYRRTPPKTKALASILAVLQIGKDWVLFDMRGLLRSVYMREAATPGQISAKDLLDTFTGCPVLNTRTGEFTFCYKLRSEGALHARKIYTKGETRTLLTSLTSENNTIALVTVDLGQRNPAAIMISRLSRKEELSEKDIQPVSRRLLPDRYLNELKRYRDAYDAFRQEVRDEAFTSLCPEHQEQVQQYEALTPEKAKNLVLKHFFGTHDPDLPWDDMTSNTHYIANLYLERGGDPSKVFFTRPLKKDSKSKKPRKPTKRTDASISRLPEIRPKMPEDARKAFEKAKWEIYTGHEKFPKLAKRVNQLCREIANWIEKEAKRLTLCDTVVVGIEDLSLPPKRGKGKFQETWQGFFRQKFENRWVIDTLKKAIQNRAHDKGKYVLGLAPYWTSQRCPACGFIHKSNRNGDHFKCLKCEALFHADSEVATWNLALVAVLGKGITNPDSKKPSGQKKTGTTRKKQIKGKNKGKETVNVPPTTQEVEDIIAFFEKDDETVRNPVYKPTGT

SEQ ID NO: 25 - protein, 769 aa, unidentified
MKKPNNIRRIREEHFEGLCFGKDVLTKAGKIYEKDGEEAAIDFLMGKDEEDPPNFKPPAKTTIVAQSRPFDQWPIYQVSQAVQERVFAYTEEEFNASKEALFSGDISSKSRDFWFKTNNISDQGIGAQGLNTILSHAFSRYSGVIKKVENRNKKRLKKLSKKNQLKIEEGLEILEFKPDSAFNENGLLAQPPGINPNIYGYQAVTPFVFDPDNPGDVILPKQYEGYSRKPDDIIEKGPSRLDIPKGQPGYVPEHQRKNLKKKGRVRLYRRTPPKTKALASILAVLQIGKDWVLFDMRGLLRSVYMREAATPGQISAKDLLDTFTGCPVLNTRTGEFTFCYKLRSEGALHARKIYTKGETRTLLTSLTSENNTIALVTVDLGQRNPAAIMISRLSRKEELSEKDIQPVSRRLLPDRYLNELKRYRDAYDAFRQEVRDEAFTSLCPEHQEQVQQYEALTPEKAKNLVLKHFFGTHDPDLPWDDMTSNTHYIANLYLERGGDPSKVFFTRPLKKDSKSKKPRKPTKRTDASISRLPEIRPKMPEDARKAFEKAKWEIYTGHEKFPKLAKRVNQLCREIANWIEKEAKRLTLCDTVVVGIEDLSLPPKRGKGKFQETWQGFFRQKFENRWVIDTLKKAIQNRAHDKGKYVLGLAPYWTSQRCPACGFIHKSNRNGDHFKCLKCEALFHADSEVATWNLALVAVLGKGITNPDSKKPSGQKKTGTTRKKQIKGKNKGKETVNVPPTTQEVEDIIAFFEKDDETVRNPVYKPTGT

SEQ ID NO: 26 - protein, 767 aa, unidentified
VIKTHFPAGRFRKDHQKTAGKKLKHEGEEACVEYLRNKVSDYPPNFKPPAKGTIVAQSRPFSEWPIVRASEAIQKYVYGLTVAELDVFSPGTSKPSHAEWFAKTGVENYGYRQVQGLNTIFQNTVNRFKGVLKKVENRNKKSLKRQEGANRRRVEEGLPEVPVTVESATDDEGRLLQPPGVNPSIYGYQGVAPRVCTDLQGFSGMSVDFAGYRRDPDAVLVESLPEGRLSIPKGERGYVPEWQRDPERNKFPLREGSRRQRKWYSNACHKPKPGRTSKYDPEALKKASAKDALLVSISIGEDWAIIDVRGLLRDARRRGFTPEEGLSLNSLLGLFTEYPVFDVQRGLITFTYKLGQVDVHSRKTVPTFRSRALLESLVAKEEIALVSVDLGQTNPASMKVSRVRAQEGALVAEPVHRMFLSDVLLGELSSYRKRMDAFEDAIRAQAFETMTPEQQAEITRVCDVSVEVARRRVCEKYSISPQDVPWGEMTGHSTFIVDAVLRKGGDESLVYFKNKEGETLKFRDLRISRMEGVRPRLTKDTRDALNKAVLDLKRAHPTFAKLAKQKLELARRCVNFIEREAKRYTQCERVVFVIEDLNVGFFHGKGKRDRGWDAFFTAKKENRWVIQALHKAFSDLGLHRGSYVIEVTPQRTSMTCPRCGHCDKGNRNGEKFVCLQCGATLHADLEVATDNIERVALTGKAMPKPPVRERSGDVQKAGTARKARKPLKPKQKTEPSVQEGSSDDGVDKSPGDASRNPVYNPSDTLSI

SEQ ID NO: 27 - protein, 763 aa, unidentified
MAKAKTLAALLRELLPGQHLAPHHRWVANKLLMTSGDAAAFVIGKSVSDPVRGSFRKDVITKAGRIFKKDGPDAAAAFLDGKWEDRPPNFQPPAKAAIVAISRSFDEWPIVKVSCAIQQYLYALPVQEFESSVPEARAQAHAAWFQDTGVDDCNFKSTQGLNAIFNHGKRTYEGVLKKAQNRNDKKNLRLERINAKRAEAGQAPLVAGPDESPTDDAGCLLHPPGINANIYCYQQVSPRPYEQSCGIQLPPEYAGYNRLSNVAIPPMPNRLDIPQGQPGYVPEHHRHGIKKFGRVRKRYGVVPGRNRDADGKRTRQVLTEAGAAAKARDSVLAVIRIGDDWTVVDLRGLLRNAQWRKLVPDGGITVQGLLDLFTGDPVIDPRRGVVTFIYKADSVGIHSEKVCRGKQSKNLLERLCAMPEKSSTRLDCARQAVALVSVDLGQRNPVAARFSRVSLAEGQLQAQLVSAQFLDDAMVAMIRSYREEYDRFESLVREQAKAALSPEQLSEIVRHEADSAESVKSCVCAKFGIDPAGLSWDKMTSGTWRIADHVQAAGGDVEWFFFKTCGKGKEIKTVRRSDFNVAKQFRLRLSPETRKDWNDAIWELKRGNPAYVSFSKRKSEFARRVVNDLVHRARRAVRCDEVVFAIEDLNISFFHGKGQRQMGWDAFFEVKQENRWFIQALHKAFVERATHKGGYVLEVAPARTSTTCPECRHCDPESRRGEQFCCIKCRHTCHADLEVATFNIEQVALTGVSLPKRLSSTLL

SEQ ID NO: 28 - protein, 761 aa, unidentified
MSKEKTPPSAYAILKAKHFPDLDFEKKHKMMAGRMFKNGASEQEVVQYLQGKGSESLMDVKPPAKSPILAQSRPFDEWEMVRTSRLIQETIFGIPKRGSIPKRDGLSETQFNELVASLEVGGKPMLNKQTRAIFYGLLGIKPPTFHAMAQNILIDLAINIRKGVLKKVDNLNEKNRKKVKRIRDAGEQDVMVPAEVTAHDDRGYLNHPPGVNPTIPGYQGVVIPFPEGFEGLPSGMTPVDWSHVLVDYLPHDRLSIPKGSPGYIPEWQRPLLNRHKGRRHRSWYANSLNKPRKSRTEEAKDRQNAGKRTALIEAERLKGVLPVLMRFKEDWLIIDARGLLRNARYRGVLPEGSTLGNLIDLFSDSPRVDTRRGICTFLYRKGRAYSTKPVKRKESKETLLKLTEKSTIALVSIDLGQTNPLTAKLSKVRQVDGCLVAEPVLRKLIDNASEDGKEIARYRVAHDLLRARILEDAIDLLGIYKDEVVRARSDTPDLCKERVCRFLGLDSQAIDWDRMTPYTDFIAQAFVAKGGDPKVVTIKPNGKPKMFRKDRSIKNMKGIRLDISKEASSAYREAQWAIQRESPDFQRLAVWQSQLTKRIVNQLVAWAKKCTQCDTVVLAFEDLNIGMMHGSGKWANGGWNALFLHKQENRWFMQAFHKALTELSAHKGIPTIEVLPHRTSITCTQCGHCHPGNRDGERFKCLKCEFLANTDLEIATDNIERVALTGLPMPKGERSSAKRKPGGTRKTKKSKHSGNSPLAAE

SEQ ID NO: 29 - protein, 746 aa, unidentified
MEKAGPTSPLSVLIHKNFEGCRFQIDHLKIAGRKLAREGEAAAIEYLLDKKCEGLPPNFQPPAKGNVIAQSRPFTEWAPYRASVAIQKYIYSLSVDERKVCDPGSSSDSHEKWFKQTGVQNYGYTHVQGLNLIFKHALARYDGVLKKVDNRNEKNRKKAERVNSFRREEGLPEEVFEEEKATDETGHLLQPPGVNHSIYCYQSVRPKPFNPRKPGGISLPEAYSGYSLKPQDELPIGSLDRLSIPPGQPGYVPEWQRSQLTTQKHRRKRSWYSAQKWKPRTGRTSTFDPDRLNCARAQGAILAVVRIHEDWVVFDVRGLLRNALWRELAGKGLTVRDLLDFFTGDPVVDTKRGVVTFTYKLGKVDVHSLRTVRGKRSKKVLEDLTLSSDVGLVTIDLGQTNVLAADYSKVTRSENGELLAVPLSKSFLPKHLLHEVTAYRTSYDQMEEGFRRKALLTLTEDQQVEVTLVRDFSVESSKTKLLQLGVDVTSLPWEKMSSNTTYISDQLLQQGADPASLFFDGERDGKPCRHKKKDRTWAYLVRPKVSPETRKALNEALWALKNTSPEFESLSKRKIQFSRRCMNYLLNEAKRISGCGQVVFVIEDLNVRVHHGRGKRAIGWDNFFKPKRENRWFMQALHKAASELAIHRGMHIIEACPARSSITCPKCGHCDPENRCSSDREKFLCVKCGAAFHADLEVATFNLRKVALTGTALPKSIDHSRDGLIPKGARNRKLKEPQANDEKACA

SEQ ID NO: 30 - protein, 735 aa, unidentified
MKEQSPLSSVLKSNFPGKKFLSADIRVAGRKLAQLGEAAAVEYLSPRQRDSVPNFRPPAFCTVVAKSRPFEEWPIYKASVLLQEQIYGMTGQEFEERCGSIPTSLSGLRQWASSVGLGAAMEGLHVQGMNLMVKNAINRYKGVLVKVENRNKKLVEANEAKNSSREERGLPPLRPPELGSAFGPDGRLVNPPGIDKSIRLYQGVSPVPVVKTTGRPTVHRLDIPAGEKGHVPLWQREAGLVKEGPRRRRMWYSNSNLKRSRKDRSAEASEARKADSVVVRVSVKEDWVDIDVRGLLRNVAWRGIERAGESTEDLLSLFSGDPVVDPSRDSVVFLYKEGVVDVLSKKVVGAGKSRKQLEKMVSEGPVALVSCDLGQTNYVAARVSVLDESLSPVRSFRVDPREFPSADGSQGVVGSLDRIRADSDRLEAKLLSEAEASLPEPVRAEIEFLRSERPSAVAGRLCLKLGIDPRSIPWEKMGSTTSFISEALSAKGSPLALHDGAPIKDSRFAHAARGRLSPESRKALNEALWERKSSSREYGVISRRKSEASRRMANAVLSESRRLTGLAVVAVNLEDLNMVSKFFHGRGKRAPGWAGFFTPKMENRWFIRSIHKAMCDLSKHRGITVIESRPERTSISCPECGHCDPENRSGERFSCKSCGVSLHADFEVATRNLERVALTGKPMPRRENLHSPEGATASRKTRKKPREATASTFLDLRSVLSSAENEGSGPAARAG

SEQ ID NO: 31 - protein, 725 aa, unidentified
MLPPSNKIGKSMSLKEFINKRNFKSSIIKQAGKILKKEGEEAVKKYLDDNYVEGYKKRDFPITAKCNIVASNRKIEDFDISKFSSFIQNYVFNLNKDNFEEFSKIKYNRKSFDELYKKIANEIGLEKPNYENIQGEIAVIRNAINIYNGVLKKVENRNKKIQEKNQSKDPPKLLSAFDDNGFLAERPGINETIYGYQSVRLRHLDVEKDKDIIVQLPDIYQKYNKKSTDKISVKKRLNKYNVDEYGKLISKRRKERINKDDAILCVSNFGDDWIIFDARGLLRQTYRYKLKKKGLCIKDLLNLFTGDPIINPTKTDLKEALSLSFKDGIINNRTLKVKNYKKCPELISELIRDKGKVAMISIDLGQTNPISYRLSKFTANNVAYIENGVISEDDIVKMKKWREKSDKLENLIKEEAIASLSDDEQREVRLYENDIADNTKKKILEKFNIREEDLDFSKMSNNTYFIRDCLKNKNIDESEFTFEKNGKKLDPTDACFAREYKNKLSELTRKKINEKIWEIKKNSKEYHKISIYKKETIRYIVNKLIKQSKEKSECDDIIVNIEKLQIGGNFFGGRGKRDPGWNNFFLPKEENRWFINACHKAFSELAPHKGIIVIESDPAYTSQTCPKCENCDKENRNGEKFKCKKCNYEANADIDVATENLEKIAKNGRRLIKNFDQLGERLPGAEMPGGARKRKPSKSLPKNGRGAGVGSEPELINQSPSQVIA

SEQ ID NO: 32 - protein, 718 aa, unidentified
VPDKKETPLVALCKKSFPGLRFKKHDSRQAGRILKSKGEGAAVAFLEGKGGTTQPNFKPPVKCNIVAMSRPLEEWPIYKASVVIQKYVYAQSYEEFKATDPGKSEAGLRAWLKATRVDTDGYFNVQGLNLIFQNARATYEGVLKKVENRNSKKVAKIEQRNEHRAERGLPLLTLDEPETALDETGHLRHRPGINCSVFGYQHMKLKPYVPGSIPGVTGYSRDPSTPIAACGVDRLEIPEGQPGYVPPWDRENLSVKKHRRKRASWARSRGGAIDDNMLLAVVRVADDWALLDLRGLLRNTQYRKLLDRSVPVTIESLLNLVTNDPTLSVVKKPGKPVRYTATLIYKQGVVPVVKAKVVKGSYVSKMLDDTTETFSLVGVDLGVNNLIAANALRIRPGKCVERLQAFTLPEQTVEDFFRFRKAYDKHQENLRLAAVRSLTAEQQAEVLALDTFGPEQAKMQVCGHLGLSVDEVPWDKVNSRSSILSDLAKERGVDDTLYMFPFFKGKGKKRKTEIRKRWDVNWAQHFRPQLTSETRKALNEAKWEAERNSSKYHQLSIRKKELSRHCVNYVIRTAEKRAQCGKVIVAVEDLHHSFRRGGKGSRKSGWGGFFAAKQEGRWLMDALFGAFCDLAVHRGYRVIKVDPYNTSRTCPECGHCDKANRDRVNREAFICVCCGYRGNADIDVAAYNIAMVAITGVSLRKAARASVASTPLESLAAE

SEQ ID NO: 33 - protein, 708 aa, unidentified
MSKTKELNDYQEALARRLPGVRHQKSVRRAARLVYDRQGEDAMVAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQEAAE

SEQ ID NO: 34 - protein, 491 aa, unidentified
VLLSDRIQYTDPSAPIPAMTVVDRRKIKKGEPGYVPPFMRKNLSTNKHRRMRLSRGQKEACALPVGLRLPDGKDGWDFIIFDGRALLRACRRLRLEVTSMDDVLDKFTGDPRIQLSPAGETIVTCMLKPQHTGVIQQKLITGKMKDRLVQLTAEAPIAMLTVDLGEHNLVACGAYTVGQRRGKLQSERLEAFLLPEKVLADFEGYRRDSDEHSETLRHEALKALSKRQQREVLDMLRTGADQARESLCYKYGLDLQALPWDKMSSNSTFIAQHLMSLGFGESATHVRYRPKRKASERTILKYDSRFAAEEKIKLTDETRRAWNEAIWECQRASQEFRCLSVRKLQLARAAVNWTLTQAKQRSRCPRVVVVVEDLNVRFMHGGGKRQEGWAGFFKARSEKRWFIQALHKAYTELPTNRGIHVMEVNPARTSITCTKCGYCDPENRYGEDFHCRNPKCKVRGGHVANADLDIATENLARVALSGPMPKAPKLK

SEQ ID NO: 35 - protein, 848 aa, unidentified
MTPSFGYQMIIVTPIHHASGAWATLRLLFLNPKTSGVMLGMTKTKSAFALMREEVFPGLLFKSADLKMAGRKFAKEGREAAIEYLRGKDEERPANFKPPAKGDIIAQSRPFDQWPIVQVSQAIQKYIFGLTKAEFDATKTLLYGEGNHPTTESRRRWFEATGVPDFGFTSAQGLNAIFSSALARYEGVIQKVENRNEKRLKKLSEKNQRLVEEGHAVEAYVPETAFHTLESLKALSEKSLVPLDDLMDKIDRLAQPPGINPCLYGYQQVAPYIYDPENPRGVVLPDLYLGYCRKPDDPITACPNRLDIPKGQPGYIPEHQRGQLKKHGRVRRFRYTNPQAKARAKAQTAILAVLRIDEDWVVMDLRGLLRNVYFREVAAPGELTARTLLDTFTGCPVLNLRSNVVTFCYDIESKGALHAEYVRKGWATRNKLLDLTKDGQSVALLSVDLGQRHPVAVMISRLKRDDKGDLSEKSIQVVSRTFADQYVDKLKRYRVQYDALRKEIYDAALVSLPPEQQAEIRAYEAFAPGDAKANVLSVMFQGEVSPDELPWDKMNTNTHYISDLYLRRGGDPSRVFFVPQPSTPKKNAKKPPAPRKPVKRTDENVSHMPEFRPHLSNETREAFQKAKWTMERGNVRYAQLSRFLNQIVREANNWLVSEAKKLTQCQTVVWAIEDLHVPFFHGKGKYHETWDGFFRQKKEDRWFVNVFHKAISERAPNKGEYVMEVAPYRTSQRCPVCGFVDADNRHGDHFKCLRCGVELHADLEVATWNIALVAVQGHGIAGPPREQSCGGETAGTARKGKNIKKNKGLADAVTVEAQDSEGGSKKDAGTARNPVYIPSESQVNCPAP

SEQ ID NO: 36 - protein, 781 aa, unidentified
MKPKTPKPPKTPVAALIDKHFPGKRFRASYLKSVGKKLKNQGEDVAVRFLTGKDEERPPNFQPPAKSNIVAQSRPIEEWPIHKVSVAVQEYVYGLTVAEKEACSDAGESSSSHAAWFAKTGVENFGYTSVQGLNKIFPPTFNRFDGVIKKVENRNEKKRQKATRINEAKRNKGQSEDPPEAEVKATDDAGYLLQPPGINHSVYGYQSITLCPYTAEKFPTIKLPEEYAGYHSNPDAPIPAGVPDRLAIPEGQPGHVPEEHRAGLSTKKHRRVRQWYAMANWKPKPKRTSKPDYDRLAKARAQGALLIVIRIDEDWVVVDARGLLRNVRWRSLGKREITPNELLDLFTGDPVLDLKRGVVTFTYAEGVVNVCSRSTTKGKQTKVLLDAMTAPRDGKKRQIGMVAVDLGQTNPIAAEYSRVGKNAAGTLEATPLSRSTLPDELLREIALYRKAHDRLEAQLREEAVLKLTAEQQAENARYVETSEEGAKLALANLGVDTSTLPWDAMTGWSTCISDHLINHGGDTSAVFFQTIRKGTKKLETIKRKDSSWADIVRPRLTKETREALNDFLWELKRSHEGYEKLSKRLEELARRAVNHVVQEVKWLTQCQDIVIVIEDLNVRNFHGGGKRGGGWSNFFTVKKENRWFMQALHKAFSDLAAHRGIPVLEVYPARTSITCLGCGHCDPENRDGEAFVCQQCGATFHADLEVATRNIARVALTGEAMPKAPAREQPGGAKKRGTSRRRKLTEVAVKSAEPTIHQAKNQQLNGTSRDPVYKGSELPAL

SEQ ID NO: 37 - protein, 767 aa, unidentified
MSEITDLLKANFKGKTFKSADMRMAGRILKKSGAQAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGPRKRTDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRGVQTTGTARNPGRSLKSNKNPSEPKRVLQSKTRKKITSTETQNEPLATDLKT

SEQ ID NO: 38 - protein, 760 aa, unidentified
MTPKTESPLSALCKKHFPGKRFRTNYLKDAGKILKKHGEDAVVAFLSDKQEDEPANFCPPAKVHILAQSRPFEDWPINLASKAIQTYVYGLTADERKTCEPGTSKESHDRWFKETGVDHHGFTSVQGLNLIFKHTLNRYDGVIKKVETRNEKRRSSVVRINEKKAAEGLPLIAAEAEETAFGEDGRLLQPPGVNHSIYCFQQVSPQPYSSKKHPQVVLPHAVQGVDPDAPIPVGRPNRLDIPKGQPGYVPEWQRPHLSMKCKRVRMWYARANWRRKPGRRSVLNEARLKEASAKGALPIVLVIGDDWLVMDARGLLRSVFWRRVAKPGLSLSELLNVTPTGLFSGDPVIDPKRGLVTFTSKLGVVAVHSRKPTRGKKSKDLLLKMTKPTDDGMPRHVGMVAIDLGQTNPVAAEYSRVVQSDAGTLKQEPVSRGVLPDDLLKDVARYRRAYDLTEESIRQEAIALLSEGHRAEVTKLDQTTANETKRLLVDRGVSESLPWEKMSSNTTYISDCLVALGKTDDVFFVPKAKKGKKETGIAVKRKDHGWSKLLRPRTSPEARKALNENQWAVKRASPEYERLSRRKLELGRRCVNHIIQETKRWTQCEDIVVVLEDLNVGFFHGSGKRPDGWDNFFVSKRENRWFIQVLHKAFGDLATHRGTHVIEVHPARTSITCIKCGHCDAGNRDGESFVCLASACGDRRHADLEVATRNVARVAITGERMPPSEQARDVQKAGGARKRKPSARNVKSSYPAVEPAPASP

SEQ ID NO: 39 - protein, 752 aa, unidentified
MSDNKMKKLSKEEKPLTPLQILIRKYIDKSQYPSGFKTTIIKQAGVRIKSVKSEQDEINLANWIISKYDPTYIKRDFNPSAKCQIIATSRSVADFDIVKMSNKVQEIFFASSHLDKNVFDIGKSKSDHDSWFERNNVDRGIYTYSNVQGMNLIFSNTKNTYLGVAVKAQNKFSSKMKRIQDINNFRITNHQSPLPIPDEIKIYDDAGFLLNPPGVNPNIFGYQSCLLKPLENKEIISKTSFPEYSRLPADMIEVNYKISNRLKFSNDQKGFIQFKDKLNLFKINSQELFSKRRRLSGQPILLVASFGDDWVVLDGRGLLRQVYYRGIAKPGSITISELLGFFTGDPIVDPIRGVVSLGFKPGVLSQETLKTTSARIFAEKLPNLVLNNNVGLMSIDLGQTNPVSYRLSEITSNMSVEHICSDFLSQDQISSIEKAKTSLDNLEEEIAIKAVDHLSDEDKINFANFSKLNLPEDTRQSLFEKYPELIGSKLDFGSMGSGTSYIADELIKFENKDAFYPSGKKKFDLSFSRDLRKKLSDETRKSYNDALFLEKRTNDKYLKNAKRRKQIVRTVANSLVSKIEELGLTPVINIENLAMSGGFFDGRGKREKGWDNFFKVKKENRWVMKDFHKAFSELSPHHGVIVIESPPYCTSVTCTKCNFCDKKNRNGHKFTCQRCGLDANADLDIATENLEKVAISGKRMPGSERSSDERKVAVARKAKSPKGKAIKGVKCTITDEPALLSANSQDCSQSTS

SEQ ID NO: 40 - protein, 747 aa, unidentified
MALSLAEVRERHFKGLRFRSSYLKRAGKILKKEGEAACVAYLTGKDEESPPNFKPPAKCDVVAQSRPFEEWPIVQASVAVQSYVYGLTKEAFEAFNPGTTKQSHEACLAATGIDTCGYSNVQGLNLIFRQAKNRYEGVITKVENRNKKAKKKLTRKNEWRQKNGHSELPEAPEELTFNDEGRLLQPPGINPSLYTYQQISPTPWSPKDSSILPPQYAGYERDPNAPIPFGVAKDRLTIASGCPGYIPEWMRTAGEKTNPRTQKKFMHPGLSTRKNKRMRLPRSVRSAPLGALLVTIHLGEDWLVLDVRGLLRNARWRGVAPKDISTQGLLNLFTGDPVIDTRRGVVTFTYKPETVGIHSRTWLYKGKQTKEVLEKLTQDQTVALVAIDLGQTNPVSAAASRVSRSGENLSIETVDRFFLPDELIKELRLYRMAHDRLEERIREESTLALTEAQQAEVRALEHVVRDDAKNKVCAAFNLDAASLPWDQMTSNTTYLSEAILAQGVSRDQVFFTPNPKKGSKEPVEVMRKDRAWVYAFKAKLSEETRKAKNEALWALKRASPDYARLSKRREELCRRSVNMVINRAKKRTQCQVVIPVLEDLNIGFFHGSGKRLPGWDNFFVAKKENRWLMNGLHKSFSDLAVHRGFYVFEVMPHRTSITCPACGHCDSENRDGEAFVCLSCKRTYHADLDVATHNLTQVAGTGLPMPEREHPGGTKKPGGSRKPESPQTHAPILHRTDYSESADRLGS

SEQ ID NO: 41 - protein, 733 aa, unidentified
QAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGPRKRTDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRDVQTPGTARKSGRSLKSQDNLSEPKRVLQSKTRKKITSTETQNEPLATDLKT

SEQ ID NO: 42 - protein, 702 aa, unidentified
MIKEQSELSKLIEKYYPGKKFYSNDLKQAGKHLKKSEHLTAKESEELTVEFLKSCKEKLYDFRPPAKALIISTSRPFEEWPIYKASESIQKYIYSLTKEELEKYNISTDKTSQENFFKESLIDNYGFANVSGLNLIFQHTKAIYDGVLKKVNNRNNKILKKYKRKIEEGIEIDSPELEKAIDESGHFINPPGINKNIYCYQQVSPTIFNSFKETKIICPFNYKRNPNDIIQKGVIDRLAIPFGEPGYIPDHQRDKVNKHKKRIRKYYKNNENKNKDAILAKINIGEDWVLFDLRGLLRNAYWRKLIPKQGITPQQLLDMFSGDPVIDPIKNNITFIYKESIIPIHSESIIKTKKSKELLEKLTKDEQIALVSIDLGQTNPVAARFSRLSSDLKPEHVSSSFLPDELKNEICRYREKSDLLEIEIKNKAIKMLSQEQQDEIKLVNDISSEELKNSVCKKYNIDNSKIPWDKMNGFTTFIADEFINNGGDKSLVYFTAKDKKSKKEKLVKLSDKKIANSFKPKISKETREILNKITWDEKISSNEYKKLSKRKLEFARRATNYLINQAKKATRLNNVVLVVEDLNSKFFHGSGKREDGWDNFFIPKKENRWFIQALHKSLTDVSIHRGINVIEVRPERTSITCPKCGCCDKENRKGEDFKCIKCDSVYHADLEVATFNIEKVAITGESMPKPDCERLGGEESIG

SEQ ID NO: 43 - protein, 661 aa, unidentified
VAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQ

SEQ ID NO: 44 - protein, 531 aa, unidentified
LEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGIITFSYKEGVVPVFSQKIVSRFKSRDTLEKLTSQGPVALLSVDLGQNEPVAARVCSLKNINDKIALDNSCRIPFLDDYKKQIKDYRDSLDELEIKIRLEAINSLDVNQQVEIRDLDVFSADRAKASTVDMFDIDPNLISWDSMSDARFSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKSFSELSSNRGLCVIEVNPAWTSATCPDCGFCSKENRDGINFTCRKCGVSYHADIDVATLNIARVAVLGKPMSGPADRERLGGTKKPRVARSRKDMKRKDISNGTVEVMVTA

SEQ ID NO: 45 - protein, 479 aa, unidentified
IPSFGYLDRLKIAKGQPGYIPEWQRETINPSKKVRRYWATNHEKIRNAIPLVVFIGDDWVIIDGRGLLRDARRRKLADKNTTIEQLLEMVSNDPVIDSTRGIATLSYVEGVVPVRSFIPIGEKKGREYLEKSTQKESVTLLSVDIGQINPVSCGVYKVSNGCSKIDFLDKFFLDKKHLDAIQKYRTLQDSLEASIVNEALDEIDPSFKKEYQNINSQTSNDVKKSLCTEYNIDPEAISWQDITAHSTLISDYLIDNNITNDVYRTVNKAKYKTNDFGWYKKFSAKLSKEAREALNEKIWELKIASSKYKKLSVRKKEIARTIANDCVKRAETYGDNVVVAMESLTKNNKVMSGRGKRDPGWHNLGQAKVENRWFIQAISSAFEDKATHHGTPVLKVNPAYTSQTCPSCGHCSKDNRSSKDRTIFVCKSCGEKFNADLDVATYNIAHVAFSGKKLSPPSEKSSATKKPRSARKSKKSRKS

SEQ ID NO: 46 - protein, 452 aa, unidentified
SPIEKLLNGLLVKITFGNDWIICDARGLLDNVQKGIIHKSYFTNKSSLVDLIDLFTCNPIVNYKNNVVTFCYKEGVVDVKSFTPIKSGPKTQENLIKKLKYSRFQNEKDACVLGVGVDVGVTNPFAINGFKMPVDESSEWVMLNEPLFTIETSQAFREEIMAYQQRTDEMNDQFNQQSIDLLPPEYKVEFDNLPEDINEVAKYNLLHTLNIPNNFLWDKMSNTTQFISDYLIQIGRGTETEKTITTKKGKEKILTIRDVNWFNTFKPKISEETGKARTEIKRDLQKNSDQFQKLAKSREQSCRTWVNNVTEEAKIKSGCPLIIFVIEALVKDNRVFSGKGHRAIGWHNFGKQKNERRWWVQAIHKAFQEQGVNHGYPVILCPPQYTSQTCPKCNHVDRDNRSGEKFKCLKYGWIGNADLDVGAYNIARVAITGKALSKPLEQKKIKKAKNKT

SEQ ID NO: 47 - protein, 425 aa, unidentified
LLDNVQKGIIHKSYFTNKSSLVDLIDLFTCNPIVNYKNNVVTFCYKEGVVDVKSFTPIKSGPKTQENLIKKLKYSRFQNEKDACVLGVGVDVGVTNPFAINGFKMPVDESSEWVMLNEPLFTIETSQAFREEIMAYQQRTDEMNDQFNQQSIDLLPPEYKVEFDNLPEDINEVAKYNLLHTLNIPNNFLWDKMSNTTQFISDYLIQIGRGTETEKTITTKKGKEKILTIRDVNWFNTFKPKISEETGKARTEIKRDLQKNSDQFQKLAKSREQSCRTWVNNVTEEAKIKSGCPLIIFVIEALVKDNRVFSGKGHRAIGWHNFGKQKNERRWWVQAIHKAFQEQGVNHGYPVILCPPQYTSQTCPKCNHVDRDNRSGEKFKCLKYGWIGNADLDVGAYNIARVAITGKALSKPLEQKKIKKAKNKT

SEQ ID NO: 48 - protein, 735 aa, unidentified
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKEF

SEQ ID NO: 49 - protein, 753 aa, unidentified
SNAPKKKRKVGIHGVPAAMIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKEF

SEQ ID NO: 50 - protein, 455 aa, unidentified
MAKKGTNRKKMIVKVMKYELKYESGCADFNEMQNELWKLQRQTREVMNRTIQLCYHWSYVQADYCKQHGCARRDVKPCDVYETNATSLDGYIYQLFKDEYPNFLMANLIATLRKAHQKYDALLFDIQEGNSSIPSFKKDQPLIFSKEAIRLPECLSDKRQITLFCFSKPYKSAHPTLDKITFAVRARSASEKSIFDHIISGKYALGESQLVYEKKKWFFLLSYKFTPESVDVNPEKVLGVDLGVVNALCAGSVENPHDSLFIKGTEAIEQIRRLEARKRDLQKQARYPGDGRIGHGTKTRVSPVYQTRDAIARMQDTLNHRWSRALIDFACKKGYGTIQMEDLSGIKALESEKPYLKHWTYFDLQSKIIYKAEEKGIRVVKVNPKCTSRRCSACGYISKENRKNQVEFLCVNCGYHHNADYNAAQNLSIPQIDRLIEKQLKEQESEENEAGANPK

SEQ ID NO: 51 - protein, 410 aa, unidentified
MAKGTLSKVMKYELRYLDGCGDFQNMQKELWTLQRQSREILNRTIQIAYHWDYTDREQFKKTGQHLDIKAETGYKRLDGYIYDSLKEDVQNFASVNVNATIQKAWAKYKSSKIDVLRGDMSLPSYKSDQPLVLHAQSMKIFSSDDDDVLQVTLFSNAYKKACNYSNIRFIIGLHDATQRTIIKKVLSGDWGIGQSQIVYKRPKWFLYLTYNFSPEQHEVNPDKILGVDLGESIAIYASSIGEYGSLRIEGGEISAFAKQLEARKRSLQKQAAYCGKGRIGHGTKSRVSDVYKMEDKIANFRNTVNHRYSKMLIDYALKHMYGTIQMEDLSGIKKETGFPKFLQHWTYYDLQQKIEAKAKEHGINFIKVDPAFTSQRCSKCGNIDSENRPSQAVFCCKKCGYKTNADFNAS

SEQ ID NO: 52 - protein, 426 aa, unidentified
MNVTKVMRYQLIYQGGGGDFESLQNQLWEFQRQTRAILNKTIQTMYLATANQEKFSEKALYHDLCAEYPDMISSTVNATLREATKKYRSSVREILAGRMSLPSYKRDHPILLHNQSVALKQGNQGSYFATISVFSRKYQQGTPGVKQPSFQLIAKDNTQRTILQRLLSGEYKLGQCQLIYIRPKWFLNVAYSFTPSEKALDQEKVLGVDLGCVYAIYASSYGNHGIFKISGDEITSFERKQAAIQNRAFKNDLTRIREIEERRKQKLEQARYCGEGRIGHGVKTRVAPAYQDEGKISRFRETINHRYSKALVDYAEKNGYGTIQMEDLSGIKSSTGFPKRLQHWTYFDLQQKIKYKAEEQGIKVVKIKPAYTSQRCSRCGHIDPANRKSQSEFKCIACGFSSNADYNASQNISMRNIEKIIQGKAN

SEQ ID NO: 53 - protein, 432 aa, unidentified
MAKGTITKVMKYELRYLGGFSDFHEMQKEVWQLQRQYREILNKTIQIALHWDYVSAQQFGESGTYLDIREETGYKTLDGYIYNCLKGAYSEMASANLNAAVQKAWKKYKNSKTQVLQGVMSLPSYKSDQPILIDKGNVKLSAEENNGRAVLTLFSRNYRDTRGLKGNVEFSVLLHDGTQKSIFRNLIDKTYALGQCQLVYERKKWFLLLTYSFTPAGHALDPEKILGVDLGECYALYASSCYAPGILKIEGGEIAEYALRLEKRKRSLQQQARYCGEGRIGHGTKTRVGVVYKAEDRIASFRETINHRYSKELVDYAVSNGYGTIQMEDLSAIQKDLGFPKRLRHWTYYDLQMKITNKAKEHGIAVVKIDPRYTSQRCSKCGHIDPANRPRQEEFCCTACGYACNADYNASQNISIKGIEKIIQKMLSAKAD

SEQ ID NO: 54 - protein, 435 aa, unidentified
MSKGMLTKVMKYTLRYVGGCGDFHEMQSILWELQKQTRAVLNKTIQIAFEWDYRSREAFQETGEYLDVHAETGYKRLDGYIYNCLKNEYADFAGKNLNAAIQTAWKKYNQSKRDIQTGKMSLPSYRSNQPLIIHNDNVMISQDMQAAPSVRFTLLSLEYKKAHDLNTNPTFEVLINDGTQRAIFEKVRSGEYKLGQCMIQYDKKKWFLLLTYSFQPEKLTLDKNKILGVDLGETIVICASSVSERGRFVIDGGEITRFATQIEARKRSQQHQAAYCGEGRIGHGTKTRVDAVYKTEDRIANFRDTINHRYSRALVNYAVKHGFGTIQMEDLSGIKSSDDFPKFLRHWTYYDLQSKIESKAKERGIAVVKVNPRFTSRRCSKCGYIDEGNRKDQAHFCCLSCGFRANADFNASQNLSIKGIDKIIEKEYNANSKQT

SEQ ID NO: 55 - protein, 442 aa, unidentified
MGKPITKTMKYQIHYIDGCGDFHNMQKELWDLQRIVRQILNKTINESYLWFVRSEQYYRDTGENLSVEEQTGYKTLDGHIYNLLKQEYTQKLVSNSLNASIQAAYKKMKDSRRDVMIGTMSLPSYRSDQPIIIYNKNIKFSSHPEHGFVVDCSLFSDAYKKSQGYEKSVKFQVSVDDNTQRSIFENILTGNYKHGQCSIVYEKKKWFLLLTYSFVPEETKLDPDKILGVDVGVVYALYASSKGNHGTFKIKGDEAITFIQRVEARKHSRQLQGTYCGDGRIGHGTKTRVQPVYNERALISNFQDTINHRYSKALIDYAKKNGYGTIQMEDLSGIKEVQQYPKYLQHWTYYDLQLKIQYKAKEAGIGFVKVTPKYTSQRCSHCGNIDEANRPKQDVFRCTVCGYERNADYNASQNLSIKGIDRIIDDQLKQMNKANPKKTENA

SEQ ID NO: 56 - protein, 435 aa, unidentified
MSGGAITKVMKYDLTYKDGYGNFKDMQEAVWKLIRDTRTILNETIKIAYHWDYLNEKSKRETGEHLDLLEETGYKRLDGYIYDDLKDRFPDFASSNLNAAIQTAWKKYKQSQKDVYIGKMTLPSYKSDQPLPINKQSIKIYDEEREHIVELNLFSTKHKKEHGLASNVRFRINLHDNTQHAIYERVLSGEYTLGQCQLLYDRPKWFFILTYSFKPAQNKLDPDKILGVDMGETCALYASTFGEQGSFVINGGEVSEYAKREEARKRSLQKQAAVCGEGRIGHGTKTRVSSVYKEQERISNFRDTINHRYSKALIEYAVKNGCGTIQMEDLSGIRQSTDFPKFLRHWTYYDLQQKIKTKAKETGIAVSMIDPRYTSQRCSRCGHIDKANRKDQAHFHCLKCGYSCNADFNASQNISIRGIDKIIQKELGAKAKQTD

SEQ ID NO: 57 - protein, 431 aa, unidentified
MKEIAKVMKYQLIYLDGGGDFYELQQTLWDLQRQTREILNKTIQSMYLATATNTAFEENALYHRFGAEYPMMAALNVNATLRTAKKRYTSTIKETLRGTMSLPSYKRDQPILLHNQTIHLALEDGQYSALFSVYSEKFQKAHEGVARPRFALMARDGTQRAILDRLLDGSYRLGQSQMTYEQKKWFLSLTYKFVPEVRELDKSKILGVDLGCVYAIYASSMQQKGIFKISGDEITEFEKRQAAMQNREPVSTLERVEQLEQRRWQKQQQARYCGEGRVGHGTGTRVAPAYRDADKIARFRDTINHRYSKALVEYAEKNGFGTIQMEDLSGIKEDTGFPKRLRHWTYFDLQTKIQYKAAERGITVVKIDPQYTSQRCSRCGYIDKANRASQEKFLCQSCGFEANADYNASQNISVEKIDKLIAKDKKKLART

SEQ ID NO: 58 - protein, 436 aa, unidentified
MGQVTKVMRYQLIYQDGGGDFYTVQQELWELQRQTREILNKTIQTMYLADANKEKFDNAAERTLNRRFCVDHPDMYTKTVTATLRKAKAKYNASQKEILAGRMSLPSYKRDQPILLNPQGFKIEEESDSFFAAIAVFSDKYKNKHPDVDVKRLRFRLVVKDGTQRAIIRRVISGEYKLGRSQLLYSKKKWFLNVTYSFEPAEKKVDPDKILGVDLGCVYAIYASSFGSPGVFKISGDEVSSFERKQAAIQNRSPKSTLERVEKIEERHKQKQQQARYCGEGRIGHGTKTRIAPVYQDEDKIARFRDTVNHRYSKALIDYAEKNGYGTIQMEDLSGIKSATGFPKRLKHWTYYDLQTKIEYKAEERGIKVVKIDPRYTSQRCSRCGYIDSGNRKSQAEFCCMACGFSCNADYNASQNISIGGIAKIIADKRKEADAK

SEQ ID NO: 59 - protein, 368 aa, unidentified
YLDIREETGYKTLDGYIYNCLKGAYSEMASANLNAAVQKAWKKYKNSKTQVLQGVMSLPSYKSDQPILIDKGNVKLSAEENNGRAVLTLFSRNYRDTRGLKGNVEFSVLLHDGTQKSIFRNLIDKTYALGQCQLVYERKKWFLLLTYSFTPAGHALDPEKILGVDLGECYALYASSCYAPGILKIEGGEIAEYALRLEKRKRSLQQQARYCGEGRIGHGTKTRVGVVYKAEDRIASFRETINHRYSKELVDYAVSNGYGTIQMEDLSAIQKDLGFPKRLRHWTYYDLQMKITNKAKEHGIAVVKIDPRYTSQRCSKCGHIDPANRPRQEEFCCTACGYACNADYNASQNISIKGIEKIIQKMLSAKAD

SEQ ID NO: 60 - protein, 437 aa, unidentified
MAEKTIVKVMKFELRYIDGAGEFSEMQKHLWELQKQTREVLNKTIQMGYALECKRFAHHDKTGQWLDDKELTGSKYKAVADYINAELKEDYNIFYSDCRNSTVRKAYKKFKDAKNKIFSGEMSLPSYRSNQPIIIHNRNVIIRGNAESALVGLKVFSDGFKALHGFPAAVNFKLCVKDGTQRAIIENVISEIYKISESQLIYDNKKWFLILAYRFTQKKNDLNPDKILGVDLGVKFAVYASSIGEYGSFRIKGGEVTEFIKRLEKRKKSLQNQATVCGDGRIGHGTKTRVADVYKARDKISNFQDTINHRYSRAIVDYARKNGYGTIQLEKLDNSIEKKGDYSPVLVHWTYYDLRTKMEYKAAEYGIKVIAVEPKYTSQRCSKCGYISSENRKTQESFECIKCGYKCNADFNASQNLSVRDIDRIIDEYLGANPELT

SEQ ID NO: 61 - protein, 434 aa, unidentified
VVNVAKGALSKVMKFELSYLDGCGDFQNMQKELWTLQRQTREILNRTIQIAYHWDYTDREHFKKTGQHLDVKSETGYKRLDGYIYDELKETVQNFASVNVNATIQKAWAKYKSSKTDVLRGDMSLPSYKSDQPLVLHAQSIKLSEDKDGPVLQVTLFSNAHKKACDYSNVRFAFRLHDATQRAIFKNVLSGEYGLGQSQIVYKRPKWFLYLTYNFSPEQHGLDPDKILGVDLGESIALYASSLGDYGSLRIEGGEVTAFAKQLEARKRSLQKQAAHCGEGRVGHGTRARVSDVYKAEDKIANFRNTVNHRYSKKLIEYAIQNRYGTIQMEDLSGIKQDTGFPKFLQHWTYYDLQQKIEAKAKENGINFIKVDPSYTSQRCSKCGNIDSDNRPSQAVFCCTKCGFRANADFNASQNLSIPEIDKIIKKERGANTK

SEQ ID NO: 62 - protein, 455 aa, unidentified
MAKKGTNRKKMIVKVMKYELKYEKGCADFNEMQNELWKLQRQTREVMNRTVQLCYHWNYVQADYCKQHGCAHRDVKPCDVYETNATSLDGYIYQLFKDEYPNFLMANLIATLRKAHQKYDALLPDIQEGNSSIPSFKKDQPLIFSKEAIHLPECLSDKRQITLFCFSKPYKSAHPTLDKITFAVRAHSASEKSIFDNIINGKYALGTSQLVYEKKKWFFLLSYKFTPESVDVNPEKVLGVDLGVVNALCAGSVENPHDSLFIKGTEAIEQIRRLEARKRDLQKQARYPGDGRIGHGTKTRVSPVYQTRDAIARMQDTLNHRWSRALIDFACKKGYGTIQMEDLSGIKAMESEKPYLKHWTYFDLQSKIIYKAEEKGIRVVKVNPKCTSRRCSACGYISKENRKNQAEFLCVNCGYHHNADYNAAQNLSIPQIDRLIEKQLKEQESEESEAGANPK

SEQ ID NO: 63 - protein, 500 aa, unidentified
MTERHDNESSKIKAEVSLLNSSVPDFEKKRHVKVLKLHILKPAGDMKWDELGALLRDARYRVFRLANLAISEAYLDFHKWRSGGNEQPKLKISQLNRNLRSMLEDEVTGKQTKMIKSDRYSKSGALPDSIVSPLSMYKLGGLTSKSKWSEVLRGKSSLPTFKLNMAIPVRCDKPGDRRIERTKNGDAEVELRICLQPYPRVIIATGRNSLGDGQRAILDRLLDNTKYSEQGYRQRCFEIKEDQRSGKWHLFVTYDFPAIEPAKNLSRERIVGVDLGAACPLYAAINTGHARLGWKHFSPLAARVRALQNQTIRRRRQILRGGKVSLSEDSARSGHGRKRKLKPISKLEGKIDRAYTTLNHQLSATVIKFAKDNGAGVVQMEDLKGLRETLTGTFLGERWRYEELQRFIRYKADEAGIEIRLVNPQYTSRRCSECGHIHKDFTREFRDKSREGNKSVRFLCPDCGFTADPDYNAARNLASLDIAAIIERQLEIQGLRKHDP

SEQ ID NO: 64 - protein, 471 aa, unidentified
MKEKSKTLVKVARLRILKPAGDMKWSELGEMLRTVRYRVFRLANLAVSEAYLGFHMYRTNRATEFKAETIGKLSRRLREMLIEEGVDEKDLSRYSQTGAVPDTVAGALGQYKIRGITSPTKWRQVVRGQAALPTFRNDMAIPIRCDKQYQRRLEKTEAGEIEVELMICRKPYPRIVLGTADLGPGQRAILERLLQNTDNSADGYRQRLFEAKQDTQTKKWWLYVTYDFPRLKEGKLNQEIVVGVDLGFSIPLYVALNIGHARLGRRHFQALGNRIRSLQRQVLARRRSIQRGGRVNISHSTARSGHGRKRKLLPTEKLRGRIEKSYSTLNHQLSASVIDFAKNHHAGTIQIEDLANLKEELAGTFIGARWRYHQLQQFLKYKAEEAGITLNQVNPRYTSRRCSECGFINIDFDRAFRDAGRTEGRVTKFLCPECGYEADPDYNAARNISILDIDKLIRVQCKKQGLTYDAH

SEQ ID NO: 65 - protein, 475 aa, unidentified
MPERPKTVNKVIWFQIHKPAGDMTWKELGNLLREARYRVFRLANLAVSEKYLSFHMWRTGQEYKSETIGKLNRRLREMLIEEGVEEESQKRFSATGALPDTVVSTLAKGKLAAITSKSKWKDVVNGKTSLPTFKLNMAIPVRCDKAEQRRLRRTESGDVELELMICKQPYPRVVLKTGKLKSGQRAILDRLVENNDNSKEGYSQRVFEIKQVENNDGSKEWRLYISYTFPKKAVEANADVAVGVDIGFSVPLVAAVNNGLERLGYNDFRALNERIRSLQRQVLVRRRSMQSGGRDYVSTPTARSGHGRKRKLLPIQTLRKRWDNAYTTLNHQLSHAVVSFAENHGAATIQIENVKSLKDELRGTFLGQRWRYFELQQFLKYKADEVGIELREVNARYTSRRCSECGYINMAFTRQARDKGRVDGKPMEFVCPECGYKAHPDYNAARNIAMLDIEQKMQVQCKQQGITYADDSEVL

SEQ ID NO: 66 - protein, 448 aa, unidentified
MTWPELGNMLRTVRYRVFRLANLAVSEAYLGFHMFRTKRAEEFKAETMGKLSRRLREMLIEEGVDEKDLSRYSQTGAVPDTVAGALSQYKIRGITSPTKWRQIVRGQVALPTFRNTMSIPVRCDKLYQRRLEQGDSGEVEVELMICRNPYPRVVLGTGDLNPGQQAILERLLQNTDNSADGYRQRLFEIKEDVQTRKWWLYVTYDFPKTTGKLNPEIVVGVDLGFSIPLYVALNSGHARLGYLHFKALGERIKSLQKQVMARRRAIQRGGRVSISHSTARTGHGVKRKLQPTEKLRGRIEKSYSTLNHQLSASVIDFAKNHHAGVIQIEDLSGLKEQLTGTFIGARWRYHQLQQFLKYKAEEAGITLKQINPRYTSRRCSECGFINMDFDRAFRDAGRTYGKVTKFLCPECGYEADPDYNAARNIATLDIEKLIRVQCEKHGLKFDAH

SEQ ID NO: 67 - protein, 477 aa, unidentified
VGKEGKRNVKVMKIRILKPCDGMTWNELGQLLRDARYRVFRLANLTVSEAYLNFHLWRTGRSQEFKKQTIGQLNRQLRNILQQEKYDDEKLNRYSKTGALPDTVCSALWQYKLMAVMKKSKWSEVIRGKSSLPTFRNDMAIPVRCDKPEQKRIEKTEQGQVEAALQVCVQPYPRVILGTHTLGDGQDAILKRLLDNQNQAIGGYRQRSFEIKYDEQKRWWLFITYDFPATEVATDKTIAVGVDLGVSVPLYAAVNNGPARLGRREFGGLGRRIRDLRNQTDARRRSIQRSGREGQSDDTARAGHGRKRKLLPIHILEGRLDKAYTTLNHQMSAAVIKFAAEQGAGIIQIENLAGLQDELRGTFIGGRWRYRQLQDFLKYKTQEMGIELRQVNPKYTSRRCSKCGFIHKDFDRDYRNRHSENGKPAQFVCPNPDCKYESDPDYNAARNLATLDIEEQIRVQCQKQGLEYDSKKDKNAL

SEQ ID NO: 68 - protein, 472 aa, unidentified
MKEKSKTLVKVARLRILKPAGDMTWSELGEMLRTVRYRVFRLANLAVSEAYLGFHMFRTQRAAEFKAETMGKLSRRLREMLIEEGVDEKELNCYSLTGAVPDTVAGALHQYKIRGITSPTKWRQVVRGQAALPTFRNDMSIPIRCDKPYQRRLEKTEAGEVEVELMICRKPYPRIVLGTADVGPGQEVILERLLQNKDNSSDGYRQRLFEAKQDRQTGKWWLYVTYDFPRPEEGELNPEIVVGVDLGFSVPLYVAINNGYARLGRRHFQALGNRIRSLQRQVLARRRSIQRGGRVNISHDTARSGHGIKRKLLPTEKLRGRIEKSYSTLNHQLSASVIDFTKNHHAGTIQIEDLANLKEVLAGTFIGARWRYHQLQQFLKYKADEAGITLKEVNPRYTSRRCSECGFIHKDFDRAFRDSGRTDGKVARFVCPECGYGPVDPDYNAAKNISTLDIEKHIRVQCKKQGLEYEVH

SEQ ID NO: 69 - protein, 470 aa, unidentified
MKEKAKTLVKVARLRILKPAGDMTWPELGNMLRTVRYRVFRLANLAVSEAYLGFHMFRTKRAEEFKAETMGKLSRRLREMLIEEGVDEKDLSRYSQTGAVPDTVAGALSQYKIRGITSPTKWRQIVRGQVALPTFRNTMSIPVRCDKLYQRRLEQGDSGEVEVELMICRNPYPRVVLGTGDLNPGQQAILERLLQNTDNSADGYRQRLFEIKEDVQTRKWWLYVTYDFPKTTGKLNPEIVVGVDLGFSIPLYVALNSGHARLGYLHFKALGERIKSLQKQVMARRRAIQRGGRVSISHSTARTGHGVKRKLQPTEKLRGRIEKSYSTLNHQLSASVIDFAKNHHAGVIQIEDLSGLKEQLTGTFIGARWRYHQLQQFLKYKAEEAGITLKQINPRYTSRRCSECGFINMDFDRAFRDAGRTYGKVTKFLCPECGYEADPDYNAARNIATLDIEKLIRVQCEKHGLKFDAH

SEQ ID NO: 70 - protein, 468 aa, unidentified
MAKKAKTMFKVTNFRILKPAGDMTWKELGQLLRDARYRTFRMANLALSEAYLNFYLLKKGDLKEYKNVKIGQIAKRLRDMLIEEGVDEEVQNRFSPKVALPAYVYSALDQFKLRGLTSKSNWKKVLRGQASLPTFRLNMSVPIRCDKPEHRRLEKTENGNVEVDLMICRKPYPRVVLETLKLDGSSKAILDRLLENEDNSPGNYRQRCFEVKQNPRSNDWWLYVTYEMPVDKDKKLDPKVIVGVDLGFSVPLYVAINNGHARLGRRHFQALGKRIHNLQNQVLARRRSIQRGGQVNLSHSTSRSGHGRKRKLQPTEKLQQKINSAYSTLNHQLSSSVIDFANNHKAGTIQIEDLETLKEQLTGTYIGRQWRYYQLQQFIEYKAKENSITVKKINPKYTSRRCSMCGHIHADFDRTFRDRSSNKGFVTKFICPECNFEADPDYNAAKNISTLDIENKIKLQCKKQKIDY

SEQ ID NO: 71 - protein, 457 aa, unidentified
MPKITRKIELLFDRSGLSEEECKEKWRFIYQINDNLYRVANRLVNQLYLADEIDDILRLSDQEYIALRKKLANKKLDEATRISLEEQMSQVMKRVNERRSAILQRPQQSFAYSVVTDSDTEGLTAKILDVLKQDVLSHYKADTKEVLKGEKSISNYKKGMPIPFAFNDSLRLYKEDGFFYLKWYNGIRFLLNFGRDASNNQLIVERCLGISKDEISYKACSSSIQIKKKGNHSKIFLLLVVDVPVEQYAQKPNMVVGVDLGLNVPIYAASNSTLERKAIGSREAFLNQRGAFQRRFRALQRLQTTKGGRGRLHKLEPLERVREAERNWVRTQNHLFSREVINFAIDVGASTIQMEKLANFGRDAQGEVREDKKYVLRNWSYFELQNLIEYKAKRAGIKVKYINPAFTSQTCSECGQLGERDSIHFKCTNPDCPNCGKDIHADYNGARNIAKSKDYIK

SEQ ID NO: 72 - protein, 468 aa, unidentified
MPTITRKIELTLLTEGLSEEQRKEQWGLLYHINDNLYKAANNISSKLYLDDHVSSMVRMKHAEYLSLLKELARAEKQKTPDADAIAELRKKVAAAEKEMTDQEHAICKYATEMSTQSLSYRFATELETNIFAKILDCLKQGVFATFNSDARDVKRGERAIRNYKKGMPIPFAWDKSLRIEKDNKDFYLRWYNGLRFLFNFGKDRSNNRLIVERCLKMDADYDGEYKLCNSSIQIAKREGKTKLFLLLVVKIPQEHVELNKKVVVGVDLGINVPAYVATNITEERKAIGDREHFLNSRMAFQRRYKSLQRLRGTAGGKGRAKKLEPLERLRKAEHNWVHTQNHLFSREVVDFAVKSHAATIHMEDLSGFGKDNDGNADERKEFVLRNWSYYELQNMIAYKAAKYGIKVEKIHPAYTSKTCSWCGQLGFREGVTFICENPECKQCGEKVHADYNAARNIANSKDIIKKNE

SEQ ID NO: 73 - protein, 468 aa, unidentified
MPTITRKIELTLCTDGLSEEQRKEQWGLLYHINDNLYKAANNISSKLYLDDHVSSMVRMKHAEYLSLLKELARAEKQKTPDADAIAELKKKVAATEKEMTDQEHAICKYATEMSTQSLSYRFSTEFETKIFAKILDCLKQGVFATFNSDAKDVKRGERAIRNYKKGMPIPFAWTDSLRIKKDNKDFYLLWYNGLRFLFNFGKDRSNNRLIVERCLKMDADYDGEYKLCNSSIQIAKREGKVKLFLLLVVSIPKEHVELNKKVVVSVDLGINVPAYVATNITEERKAIGDREHFLNSRMAFQRRYKSLQRLKGTTGGKGRTKKLEPLERLRKAEHNWVHTQNHLFSREVVDFAVKTHAATIHMEDLSGFGKDNDGNADERKEFVLRNWSYYELQNMISYKAAKYGIKVEKIRPAYTSKTCSWCGQHGFREGVTFICENPACKQCGEKVHADYNAARNIANSKEIIKKNE

SEQ ID NO: 74 - protein, 469 aa, unidentified
MPTITRKIELTLCTEGLSDQERKDQWNLLYHINDNLYRAANNISSKLYLDDHVGSMVRLKHAEYLSLLRALEKAKKQKAPDEEVIAELSQQVATAEQEMDEQAKAICQYATEMSTQTLSYRFATELETNIFGQILTCLRQGVFSTFNSDARDVKRGERSIRTYKKGMPIPFPWNDSLRIGFEDGEFYLRWYNGLRFRFDFGKDRSNNCLIVQRCMKMDKDYEGDYKLCNSSIQMVKREGKPKFFLLLVVNIPQERVELNKNIVVGVDLGINAPAYVATNTTPERKQIGDREHFLNERMAFQRRFKSLQRLKGTTGGRGRAKKLEPLERLRKAEQNWVHTQNHLFSREVIDFAVKARAATIHMEDLSGFGKDNDGNADERKEFVLRNWSYYELQNMITYKAAKYGIKVEKIRPAYTSKTCSWCGHQGFREGITFICENPECKKFGEKEHADYNAARNIANSKEIIKNNEE

SEQ ID NO: 75 - protein, 468 aa, unidentified
MPTITRKIELTLLTEGLSEEQRKEQWGLLYHINDNLYKAANNISSKLYLDDHVSTMVRMKHAEYLSLLRELARAEKQKKPDVDAIAELREKVTAAEKEMSDQERAICTYATEMSTQSLSYRFATEIETNIFAKILDCLKQGVFATFNSDARDVKRGERAIRNYKKGMPIPFAWDKSLRIEKDNKDFYLRWYNGLRFLFNFGKDRSNNRLIVERCLKMDADYDGEYKLCNSSIQIVKREGKVKLFLLLVVSIPQEHVELNKKIVVGVDLGINVPAYVATNITEERKAIGDREHFLNSRMAFQRRYKSLQRLKGTAGGKGRTKKLEPLERLRKAEHNWVHTQNHLFSREVVDFAVKSHAATIHMEDLSGFGKDNDGNADERKEFVLRNWSYYELQNMIAYKAAKYGIKVERIRPAYTSKTCSWCGQLGFREGVTFICENPECKQCGEKVHADYNAARNIANSKDIIKKNE

SEQ ID NO: 76 - protein, 463 aa, unidentified
MPTMTRKIELKLCTEGLSDEERKAQLGLLYHINDNLYKAANNISSKLYLDDHVSSMVRLKHAEYLSLLNEFEKAKKKGDEEQIVELSLRVAAAEKELTDQELAICKYATEMSTDTLAYRFANEIEINVFGQILACLKQGIHSTFKKDAADVKRGERAIRNFKKGMPIPFPWSKSIRIENEGSDFYLRWYNGLRFRFDFGKDRSNNRLIVSRCLNLDPDFEDEYKLSNSSLQMVKRDGRPKLFLLLVVNIPQENVELNKKIVVGVDLGINSPAYVATNITMERQRIGSRDTFLNARMAIQRRFQSLQKLQNTAGGRGRKKKLEPLERLKETERNWVRTQNHLFSRDVVQFAVKTRAATIHMEDLSGFGKDDDGNADEKKEFVLRNWSYYELQTMIKYKAAKYGIKVEKIRPAYTSRTCSWCGHEGDRKGETFICENPECEKYGKKENADYNAARNIANSTDIIK

SEQ ID NO: 77 - protein, 468 aa, unidentified
MPTITRKIELTLCTEGLSDEQRKEQWGLLYHINDNLYKAANNISSKLYLDEHVSSMVRMKHAEYLSLLKELARAEKQQTPDEGLIAELSRKLSAAEKEMADQELAICKYATEMSTQTLSYNFAKEIETNIFGQILTCLRQGVYATFNSDAKDVKRGERAIRNYKKGMPIPFPWNNSLKIESDSGEFYLRWYNGLRFLLTFGKDRSNNRMIVNRCMKMDEDFEGEYKLCNSSIQLAKRDGKPKLFLLLVVNIPQEHVKLNKKIVVGVDLGVNVPAYVATNITEERKAIGDREHFLNTRMAFQRRYKSLQRLKGTAGGKGRTKKLEPLERLRDAERNWVHTQNHLFSREVVNFAVQARAATIHMEDLSGFGKDKDGNADEKKEFVLRNWSFYELQNMIAYKSAKYGIKVVKIRPAYTSKTCSWCGQQGDRKSTTFICENPKCKHYGESIHADYNAARNIANSNDIVKENE

SEQ ID NO: 78 - protein, 467 aa, unidentified
MPKITRKIEMTLCTEGLSDEQRKEQWGLLYHINDNLYKAANNISTKLYLDEHVSSMVRMKHADYLSLLKELAKAEKKSPDEDLIAELREKLAAAEQEMTDQELAICKYATEMSTQTLAYKFATEIEINVFGQILACLKQAAQSNFKSDAKDVKRGERAIRNYKKGMPIPFPWNDNIRIDADGDEFYLRWYNGLRFHLTFGKDKSNNRMIVKRCLKMDKDFEGEYKLCNSSIQMVKRDGKPKLFLLLVVNIPQEHVELNKNVVVGVDLGVNVPAYVATNITEERKAIGEREHFLNTRMQIQRRYKSLQRLKATAGGKGRTKKLEPLERLRKAEHNWVHTQNHLFSREVVNFAVQTHAATIHMEDLSGFGKDDDGNADEQKEFVLRNWSFYELQNMIAYKAAKYGIKVEKVKPAYTSKTCSWCGQLGFRQGVTFICENPACKQCGEKVHADYNAARNIANSKDIIKKNE

SEQ ID NO: 79 - protein, 469 aa, unidentified
MPTITRKIELHLCTDGLTDEQQKAQRLLLYHINDNLYKAANNVSSKLYLDEHVSSMVRLKHDEYLSLSRELARAEKKHDDELTTELRGKLAAAEREMTDQELAICKYATEMSTQSLSYRLVTELETKIFAKILDCLKQGVYATFNSDARDVKRGERAIRNYKKGMPIPFAWNDSVRIEYDEKEKDFYLRWYNDIRFKFHFGRDRSNNRLIVSRCLKLDKDYEGDYQLCNSSIQIVKRDGSTKFFLLLVVKIPQEHVELNKRIVVGVDLGINYPAYVATNCTEERMYIGDREHFLNTRMQFQRRYKSLQKLKGTAGGKGRSKKLEPLERLRNAERNWVHTQNHLFSLKVVNFAVQTHAATIHLEDLSGFGKDDDGNADERKEFVLRNWSYYELQSMIEYKAKKYGIKVEKIRPAYTSQTCSWCGQRGFRQGVTFICENPECKKCGEKENADYNAARNIANSKDVIKDKNE

SEQ ID NO: 80 - protein, 491 aa, unidentified
TPFVLYFQNYSLSLRQHITLYSMPTITRKIELTLCTEGLSDQERKDQWNLLYHINDNLYRAANNISSKLYLDDHVGSMVRLKHAEYLSLLRAMEKAKKQKAPDEEVIAELSQQVAAAEQEMDEQAKAICQYATEMSTQTLSYRFATELETNIFGQILTCLRQGVFSTFNSDARDVKRGERSIRTYKKGMPIPFPWNDSLRIGFEDGEFYLRWYNGLRFRFDFGKDRSNNRLIVQRCMKMDKDYEGDYKLCNSSIQMVKREGKPKFFLLLVVNIPQERVELNKNIVVGVDLGINAPAYVATNTTPERKQIGDREHFLNERMAFQRRFKSLQRLKGTTGGRGRAKKLEPLERLRKAEQNWVHTQNHLFSREVIDFAVKARAATIHMEDLSGFGKDRDGNADERKEFVLRNWSYYELQNMITYKAAKYGIKVEKIRPAYTSKTCSWCGHQGFREGITFICENPECKKFGEKEHADYNAARNIANSKEIIKNNEE

SEQ ID NO: 81 - protein, 469 aa, unidentified
MPTITRKIELHLCTEELSDEQQKAQRLLLYHINDNLYKAANNVSSKLYLDEHVSSMVRLKHDEYLSLLRELARAEKKADDELATQLREKLVAAEREMTDQELAICKYATEMSTQSLSYRFVTELETKIFAKILDCLKQGVYATFNSDSRDVKRGERAIRNYKKGMPIPFAWDKSVRIEYEEKEKDFFLRWYNDIRFKFHFGRDRSNNRLIVSRCMKLDKDYEGDYQLCNSSIQIVKRDGSTKYFLLLVVKIPQEHVELNKKIVVGVDLGINYPAFAATNCTEERMSIGDREHFLNTRMQFQRRFKSLQRLKGTTGGKGRNKKLEPLERLRKAEHNWVHTQNHLFSLKVVNFAVQAHAATIHLEDLSGFGKDDDGNADERKEFVLRNWSYYELQNMIKYKAKKFGIQVEKIRPAYTSQTCSWCGQRGFRQGITFICENPECKKCGEKENADYNAARNIANSKDIIKDKDE

SEQ ID NO: 82 - protein, 460 aa, unidentified
MPIITRKIELHISKEGLSAEDYKAQWQYLRQINDNLYMAANRVSSHCFLNDEYKYRLCLQIPDYIDIEKQLKDSKRARLSKEELGQLKKRKKELENTVKGRFQDEFEKNSLYTIISNEFGEIIPGQILTCLRQCVQSKYNRAKEELEKGERAISTYKKGMPIPFPINKSIRLQKQGEDFVLKWYNKIVFKLHFGRDRSNNRVIVERLIQSALNDKQKGEDYVMNNSSIQLVEKDKMTKIFLLLSMDIPTQKRKLDSELVLGVDLGLNFPLYYATNQSANIHDHIGDKDIFLKERMVFQRRFKELQRLQCTQGGRGRKKKLEPLEKLRDKERNWVRTKNHIFSREVIKVALHLGAGTIHLENLHNFGKDGNGELKNSKKFVFRNWSYFELQSMIEYKAKMEGITVKYVNPAYTSQTCSVCGMIGERKEQAVFRCMNSSCLEYGKEVNADFNAARNIAKAKM

SEQ ID NO: 83 - protein, 466 aa, unidentified
MPTITRKIELTLCTDGLSDDLRKDQWQLLYHINDNLYKAANNISSKLYLDEHVASMVRLKHAEYLGLIKELAKARKRADDEAVRDLCSKLAVAEQEMNEQAKAICDYATEMSTQTLSYNFAKEIETNIFGQILTCLRQGVLLNFNSDARDVKRGERAIRNYKKGMPIPFPWNDTIKIVSEGDEFYLRWFSGLRFHLNFGKDRSNNRMIVRRCLKMEQDFDEEYKISNSSIQVAKRDGKQKLFLLLVVQIPQEQVVLNKKIVVGVDLGVNVPAYVATNITEERKAIGDREHFLNTRMQFQRRYKSLQRLKTTEGGRGRAKKLEPLERLRKAEHNWVHTQNHLFSREVVNFALQTQAATINMEDLSGFGKDNDGNADECKEFVLRNWSYYELQNMIVYKASKYGIRVQKIRPAYTSKTCSWCGHMGFREGVTFICENPDCKQFGEKVHADYNAARNIANSKEIIKNDE

SEQ ID NO: 84 - protein, 444 aa, unidentified
MSKTVTKTVKIALICEHTNKYGEKVDYKDINKLLWKLQKQTRELKNKTIQLCWEYNNFSCDYYKEHHEYPNMEDILKYKRINGFVENKLKTVNDLYSSNCSTTILSTCNEFQNYRSEFLKGTRSINSYKSDQPLDLHKGAIKLEHDGKDFYVSLKLLKRSAFNAMEFKGSDIRFKLNVKDKDKSTLKILESCYDKIYSISASKMTYDRKAGKWFLLLAYSFTPAKTENLDPEKILGVDLGIKIPICASVYGDLDRLTIEGGKIEEFRRRVEARKRSLQKQGKQCGDGRIGHGTKKRIKPITDIGDKIARFRDTENHIYSRYLIEYAVKKGCGTIQMEKLEGITREKDIFLKNWTYFDLQKKIEYKAKEKGIKVVYIEPAYTSKRCSSCGFIDTDNRLDQAHFKCLKCGFNENADYNASQNIGIKNIDKIIKEEHKSASDKLTSE

SEQ ID NO: 85 - protein, 439 aa, unidentified
VIILTKVVKLYLISEQINKEGQKIDYQRINSILWDLQKQTRDIKNRTVQLCWEWMNFSSDYCKTQEEYPKERDILGYTLEGYVYDYFKTGYDLYTGNISTSSREVCSSFKNVKKEILKGERSILSYKANQPLDLHKKAISLEYDNFNFFVKLKLLNRTGKKKYDITEDINFKIQVNDKSTRTILERCYDKEYKISGSKLIYEKKKKLWRLNLCYSFENSQVETLEKDKILGIDLGIVYPLMASIYGEYDRFSIKGGEIEEFRRRTEARKRSILQQTKYCGDGRIGHGRNKRTQPAYKINDKIARFRDTANHKYSRALIEYAVKKNCGIIQMENLTGISDNTDCFLKDWSYYDLQTKIENKAKEMGIKVVYIKAQYTSQRCSRCGYIDVNNRIRQALFKCQNCGYETNADYNASQNIGMYDIENIIEETLKIQSANVKQS

SEQ ID NO: 86 - protein, 437 aa, unidentified
MTKVTKVYLISEQIDKDGNKIDFKKISELLWNLQMQTRDIKNKCVQLCWEWLNFSSDYYKKSEEYPKEKDTLGYTLSGFVYDRIKNGSDLYSSNLSTSSRDTCTAFSNYKKEMLKGERSVLSFKANQPLDIHNKAIKLSYENGNFFVALKMLNRAGKEKYGIKDDLRFRMQVRDKSVRTILERLMNDEYKVSASKLMYDKKKKLWKLNLCYSFDNHVISTLDTEKIMGVDLGVVYPIMASVNGDYARFSIKGGEIEAFRSRVEARRRSLLNQSRYCGDGRIGHGRKKRTEPATQIADKIARFRDTTNHKYSRALIDYAIKNGCGTIQMEKLTGITSSAEHFLKEWSYFDLQTKIESKAKEAGIKVVYINPKFTSQRCNKCGYIHTDNRPVQARFCCQKCGYEENADYNASQNIGTKHIDVIIEETLKMQCEPETPTE

SEQ ID NO: 87 - protein, 428 aa, unidentified
MNKVVKLALICEQSDKDNSPVDYKKINEILWELQKQTREIKNKAIQYCWEYNNFSSDYYKKFNEYPKEKDILSYTLVGFVNDKFKTGNDLYSGNCSTTVRNACTEFKNSKKELIKGSRSIINYRSNQPLDIHNKCIRIEFENNCFYTYLKLLNRPAFKKYNFANTEIKFKILVRDNSTKTILERCISNEYEIAASKLLYDQKKKCWFLNLVYAFEIKSNNSLDPNKILGVDLGIHYPICASVYGSLDRFTIDGGEIDEFRRRVESRKISMLKQGKNCGDGRIGHGIKARNKPVYNIEDKIARFRDTANHKYSRALIEYAVKHTCGTIQMEDLTGITDIANRFLKNWSYYDLQTKIEYKAKEAGINIVYIDPKNTSRRCSKCGYIDKENRETQSRFICLKCGFKENADYNASQNIGIKDIDKLIKEDVH

SEQ ID NO: 88 - protein, 443 aa, unidentified
VTLLVKVVKIYLISEQFDKAGNQIDYKEVNKILWELQKQTREAKNKTVQLLWEWNNFSSDYVKASGIYPKAKDIFGYSSVHGQANKELRTKLALNSSNLSTTTMDVCKIFNTYKKEVWEGKRSVPSYKSDQPLDLHKESIKLIYENNEFYVRLALLKKAEFAKYGFKDGFRFKMQVKDNSTKTILERCFDEVYKINASKLLYDQKKKKWKLNLSYSFDNKNISELDKEKILGVDVGVNCPLVASVFGDRDRFIIKGGEIEKFRKSVEARRRSMLEQTKYCGDGRIGHGRKKRTEPALNIGDKIARFRDTTNHKYSRALIEYAVKKGCGTIQMEKLTGITSKSDRFLKDWTYYDLQTKIENKAKEVGINVVYIAPKYTSQRCSKCGYIHKDNRPNQAKFRCLECDFESNADYNASQNIGIKNIDKIIEKDLQKQESEVQVNENK

SEQ ID NO: 89 - protein, 428 aa, unidentified
MNKVVKLALICEQSDKNNSPVDYKKVNEILWELQKQTREIKNKTIQYCWEYYNFSSDYYKKFNKYPKEKDILSYTLWGFINDKFKTGNDLYSGNCSATTKKVIKEFKNSKKELIRGSRSIINYKSNQPLNIHNKCIHLQFKNNNFYVSINLLNRRSFKKYNFANTAIKFKILVRDNSTKAILERCISNEYKISESQLIYNKKKKCWFLNLSYAFEIKSNNSLDPNKILGVDLGIHYPICASVYGSLDRFTIDGGEIDEFRRRVESRKISMLKQGKNCGDGRIGHGIKARNKPVYNIEDKIARFRDTANHKYSRALIEYAVKNNCGTIQMEDLTGITDNANRFLKNWSYYDLQTKIEYKAKEASINVVYINPENTSRRCSKCGYIDKENRKTQSSFICLKCGFKENADYNASQNISIKDIDKLIKEDVH

SEQ ID NO: 90 - protein, 443 aa, unidentified
VTLLVKVVKIHLISEQFDKAGNRIDYEEVNKILWELQKQTREAKNKTVQLLWEWNNFSSDYVKASGIYPKAKDIFGYSSVHGQANKELRTKLALNSSNLSTTTMDVCKNFNTYKKEVWKGKRSVPSYKSDQPLDLHKDSIKLIYENNQFYVRLALLKKAEFAKYGFKDGFHFKMQVKDNSTKTILERCFDEVYKINASKLLYDQKKKKWKLNLSYSFDNKNISELDKEKILGVDVGVSYPLVASVFGDRDRFKIKGGEIEKFRKSVEARRRSMLEQTKYCGDGRIGHGRKKRTEPALNIGDKIARFRDTTNHKYSRALIEYAVKKGCGTIQMEKLTGITSKADRFLKDWTYYDLQTKIENKAKEVGINVVYIAPKYTSQRCSKCGYIHKDNRPNQAKFRCLECDFESNADYNASQNIGIKNIDKIIEKDLQKQESEVQVNENK

SEQ ID NO: 91 - protein, 449 aa, unidentified
LIWKDALGGIILTKIVKLYLISEQIDKDGNRVDYKEINSILWNLQKQTRDIKNKTVQLCWEWMNFSSDYYKKNELYPNEKEILNLTLRGYAYDHFKQGYDLYSSNISVLTEAVCGAFKNAKKEMLNGEKSVLSYKAEQPLDIHKKCIKLEYDKNFYVKLKMLNKAGKKKYGIEDDLNFKIQVEDKSTRTILERCIDGEYVVSGSKLIYDKKKKLWKLNLCYSFKANEIESLDKNKILGIDLGIACPLMASVNGEFDRFSIKGGEIETFRKRIEARKRSVLHQTKYCGDGRIGHGRNKRTEPAYKINDKIARFRDTANHKYSRALIDYAIRKNCGMIQMENLTGISDKKEHFLKEWSYYDLQTKIENKAKEKGIKIVYINPEYTSQRCSKCGYIDANNRELRAVFKCQKCGFEADADYNASQNIGIKNIEDIIENTLKISSANEKQTKNT

SEQ ID NO: 92 - protein, 462 aa, unidentified
VFYSTFLCYILTKYIDFSANECYNINTSSEVKQLMNKVVKLALICEQSDKDNSPVDYKKINEILWELQKQTREIKNKAIQYCWEYNNFSSDYYKKFNEYPKEKDILSYTLVGFVNDKFKTGNDLYSGNCSTTVRNACTEFKNSKKELIKGSRSIINYRSNQPLDIHNKCIRIEFENNCFYTYLKLLNRPAFKKYNFANTEIKFKILVRDNSTKTILERCISNEYEIAASKLLYDQKKKCWFLNLVYAFEIKSNNSLDPNKILGVDLGIHYPICASVYGSLDRFTIDGGEIDEFRRRVESRKISMLKQGKNCGDGRIGHGIKARNKPVYNIEDKIARFRDTANHKYSRALIEYAVKHTCGTIQMEDLTGITDIANRFLKNWSYYDLQTKIEYKAKEAGINIVYIDPKNTSRRCSKCGYIDKENRETQSRFICLKCGFKENADYNASQNIGIKDIDKLIKEDVH

SEQ ID NO: 93 - protein, 430 aa, unidentified
LISEQIDKDGNRVDYKEINSILWNLQKQTRDIKNKTVQLCWEWMNFSSDYYKKNELYPNEKEILNLTLRGYAYDHFKQGYDLYSSNISVLTEAVCGAFKNAKKEMLNGEKSVLSYKAEQPLDIHKKCIKLEYDKNFYVKLKMLNKAGKKKYGIEDDLNFKIQVEDKSTRTILERCIDGEYVVSGSKLIYDKKKKLWKLNLCYSFKANEIESLDKNKILGIDLGIACPLMASVNGEFDRFSIKGGEIETFRKRIEARKRSVLHQTKYCGDGRIGHGRNKRTEPAYKINDKIARFRDTANHKYSRALIDYAIRKNCGMIQMENLTGISDNKEHFLKEWSYYDLQTKIENKAKEKGIKIVYINPEYTSQRCSKCGYIDANNRELRAVFKCQNCGFEADADYNASQNIGIKNIEDIIENTLKISSANEKQTKNT

SEQ ID NO: 94 - protein, 429 aa, unidentified
LVKVVKIYLISEQVDEQGKDVDYNTICGVLWDLQWETREIKNKTVQLCWEWSGFSSDYYKKYGEYPKEKNLLDYTMGGFVYDKLKSKYHLYTANLSTTSQNTCGIFRTYKVDFVKGNRSVLSFKADQPLDVHKKSISIDRIDDNYFVKLKLLNKSGIQKYGIRDDFHFRMLVKDNSTKTILERCVGGDYKAAASKIIYDKKKKMWCLNLSYEFDVNTAKDLNKNRILGIDIGIVYPVVASVNGELDRFVIQGGEIETFRRRVENRKKSLLKQTKYCGDGRIGHGRNKRTEPVDIISDQIARFRNTANHKYSRAVIDYAVRKQCGTIQMENLKGITDKSDRFLKNWSYYDLQQKIEYKAKEKGINVVFINPKYTSQRCSRCGYIDSANRPKLPNQSKFLCIKCGFTENADYNASQNIALYNIEKLIDAEA

SEQ ID NO: 95 - protein, 483 aa, unidentified
LHETEKSLKFAEKYIAMPTITRKIELTLCTEGLSDEQRKEQWGLLYHINDNLYKAANNISSKLYLDEHVSSMVRMKHAEYLSLQKELARAEKQKVDDAIIVELTRKLAVAEKEMTDQELAICKYATEMSTNTLAYNFAKEIETKIFGQILACLENNAHALFVDDSPNVRRGERAIRNYKKGMPIPFPWNRSIKIEADGGEFYLRWYNGLRFLLTFGKDRSNNRLIVKRCMKMDEVFEGEYKLCNSSIQLAKRDGKPKLFLLLVVNIPQEHVELNKNIVVGVDLGVNVPAYVATNITEERKAIGDREHFLNTRMTFQKRYKSLQRLKGTAGGKGRTKKLEPLERLRDAERNWVHTQNHLFSREVVNFAVQARAATINIEDLSGFGKDNDGNADEKKEFVLRNWSYYELQNMITYKASKYGIKVEKIRPDYTSKTCSWCGQQGFREGVTFICENPECKQHGEKIHADYNAARNIANSKDIIKKNE

SEQ ID NO: 96 - protein, 467 aa, unidentified
MAETKRLQKVAKFQIVKPVNMSWDELGRMLRDVRYRLSRLANMAVSETYQNLHQRYRLKNQDAPKSLKIGQLSRNLRKILREEGVEEENLSKYSKTCVLPDTITGAFSRYKLSSIDWRKVLTGKISVPNYKTNLSIPIRCDKPHQRRLELTETGEIEADLMICNKPYPRVLLSTRTISDGQRTVLERLVSNKTNFLPGYRHRFFEVKEKKGKWELSVTYDFPKAEATRLHPDIIVGVDLGWSVPLYAAINNGYARIGYRKFEPLAKRIKHLQKQIKGRRFSTQKGGVKDLAQPTARAGHGRKRILKPIEKLEYKIDNAYTTLNHQLSHCVVEFAKNNGAGLIQIENLEGLKDDLSGTFIGQNWRYNQLQNFIKYKADEAGIKVHPVNPCYTSRRCSHCGFIHISFDREYRDKNRKNGKATMFECPKGCKPLNADYNAAKNLATFDIEEKIRLQCKQQSIEYKELPKD

SEQ ID NO: 97 - protein, 472 aa, unidentified
MPGTEKRLQKVATFEIVKPVNMSWPEFGKMLRDVRYRYWRLANMAVCENYMRFYQWRTQQTDANDRYKVKTLNRILRKMLIEEKNADEKELSRYSRDGAVSGYICGAFEKTKLSAVKSSSKWKKVIAGKESLPLFKKDLAIPINCSDHQPRLIERTQSGEYEVDLRICQQPYPRVLLSTAKISDGQKAILERLVSNETNSLPGYRHRFFEIKEKRNKWYLSVSYDFPKIDATRLHPNIIVGVDLGWSVPLYAAISNGYARIGYRKLKALGDRIKALQRQTIARRRSIQRTGEQDLSAPTARSGHGRKRILHPIEKLEGKIDNAYKTLNHQLSHCVIEFAKNHGAGLIQVENLKGLAEELSGTFIGQNWRYNQLQEFIKYKAKEAGIEVKEVNPCYTSRRCSECGFIHKEFTFEYRQANKKTDKATMFECPKCGYKAIADYNAARNLANPDIAEKIRLQCKEQGIEYKELPKD

SEQ ID NO: 98 - protein, 468 aa, unidentified
MPTITRKIELHFCTEGLSDEKQKEQRQLLYHINDNLYKAANNISSKLYLDEHVSSMVRLKHADYLSLQRELARAEKQKTPDDELITELSRKLSAAEKEMTDQELAICKYATEMATSTLAYNFAKEMETEIFGQILACLENNAHAVFVDDSLSVKRGERAIRNYKKGMPIPFPWNKNIKIETKDCEFYLRWYNGIRFRLHFGKDRSNNRLIVQRCLKLDDNFESEYKLCNSSIQLDKRDGKTKLFLLLVVNIPQEHVELNKNIVVGVDLGLNYPAYVATNSTEERKYIGDRDHFLKIRMQFQSRYKSLQRLKGTAGGKGRAKKLEPLERLRKAERNWVHTQNHLFSRDVVNFAVQTHAATIHMEDLSGFGKDNDGNADEKKEFVLRNWSYYELQSMIEYKAAKYGIKVEKIRPAYTSKTCSWCGQQGDRKSTTFICENPECKHYGESIHADYNAARNIANSKDIVKKNE

SEQ ID NO: 99 - protein, 453 aa, unidentified
MSKITRKIEIIPDIDGITHEESNKKCYNTFYKFDRKLYKVANLLVSQLYGLDNLLSLMRLQNDEYVKCQSKLSFKSITDATKEEIKKRMQEIDAELVSMKNDIAPKHPQTYSYRAVTSSEYAKDIPSDILNNLKQDVYQHFNENKKEQIRGERSLATYKKGMPIPFSFEKRHVIICDGDNYYLPWFEDTRFRLNFGRDRSNNRAIIDNCIKTKKYKLCAAAKIQLKERKLFLLITVDIPKAESVPVKGKVMGVDLGVINPAYVAVNDGPERSRIGNGEAFQKQRDVFRRRFRELQRSQLTQGGHGRKHKTKATEILRGKERNWVQTENHRISREIVNLASRWKVETIQMESLKGFGKNQEGEVEYNHKRLLGRWSYFELQKDIEYKAAMAGIAVQYVNPAYTSQTCHVCGQRGNRIERDTFICTNPECTCYNQAQDADMNAAINIAKSKDVIK

SEQ ID NO: 100 - protein, 468 aa, unidentified
MPTITRKIELTLCTDGLSDEERKAQWGLLYHINDNLYKAANNISSKLYLDEHVSSMVRLKHAEYLSLQKELAKAERQKMPDVDVIEELRERLSAAEQEMSDQELAICKYATEMSTNTLAYRFATEIETNIFGQILARLENNAQAVFLTDAPDVKRGERAIRNYKKGMPIPFPWNNSIKIECEGGEFYLRWYSGLRFHFNFGKDRSGNRLIVQRCLKLDKEYDGEYKLCNSSIQMVKRDGSTKFFLLMVVNIPQEYVELNKHIVVGVDLGINVPAYVATNITPERKAIGDREHFLNTRMAFQRRYKSLQRLKTTAGGKGRTKKLEPLERLRQAEHNWVHTQNHLFSREVVNFALQTHAATIHLEDLSGFGKDSDGNADERKEFVLRNWSYYELQNMITYKAAKYGIRVEKIRPAFTSRTCSCCGHEGFREGVTFICENPECQQFGEKVHADYNAARNIANSKDIIKKNE

SEQ ID NO: 101 - protein, 468 aa, unidentified
MPTITRKIELTLCTEGLSEEQRKEQWGLLYHINDNLYKAANNISSKLYLDDHVSSMVRMKHAEYLSLLKELARAEKQKTPDADAIAELRKKVAAAEKEMTDQEHAICKYATEMSTETLAYKFATEIETNVFGQILACLKQAAQSNFKNDAKDVKRGERAIRNYKKGMPIPFPWNDSLRIEKDNKDFYLRWYNGLRFLFNFGKDRSNNRLIVERCLKMDADYDGEYKLCNSSIQIAKREGKVKLFLLLVVSIPQEHVELNKKVVVGVDLGINVPAYVATNITEERKAIGDREHFLNTRMAFQRRYKSLQRLKGTAGGKGRTKKLEPLERLRKAEHNWVHTQNHLFSREVVDFAVKTHAATIHMEDLSGFGKDNDGNADERKEFVLRNWSFYELQNMITYKAAKYGIKVEKIRPAYTSKTCSCCGRQGFRSGVTFICENPECKQYGEKVHADYNAARNIANSKEIIKKNE

SEQ ID NO: 102 - protein, 480 aa, unidentified
MKNNVEEKRPDKEKRLTKVATFQIVKPVNMSWSEFGKMLRDVRYRLSRLANMAVSEAYQNLHQRYRLKNQNAPKSVKIGQISRDLRKILLEEGLEEENLSKYSKMCVLPDTITGAFSRYKLSTIDWRKVLTGKISIPNYKANLSIPIRCDKPQQRRLERTETGEIEVDLMICNKPYPRVLLSTRTISDGQRSVLERLVLNNANSLPGYRHRIFEIKEKRNEWYLSVTYDFPKAETTKLHSDIIVGVDLGWSVPLYAAINNGYARIGYKQLKPLGDSIKALQRQTIARRRSIQRGGTQDLAAPTARSGHGIKRILQPIEKLEGKIDNAYKTLNHQLSHCVIEFAKNHGAGVIQIENLKGLAEELSGTFIGQNWRYYQLQEFIKYKAKEAGIIVKEVNPFYTSRRCSECGYIHKDFTFEYRQANRKNGKSTMFECPKKEEKGCKPLNADYNAARNLATSDIEDKIRLQCKEQGIEYKEIKEK

SEQ ID NO: 103 - protein, 442 aa, unidentified
VTLLVKVVKIHLISEQFDKAGNRIDYKEVNKILWELQKQTREAKNKTVQLLWEWNNFSSDYVKASGIYPKAKDIFGYSSVHGQANKELRTKLILNSSNLSTTTMDVCKIFNTYKKEVWEGKRSVPSYKSDQPLDLHNDSIKLIYENKEFYVRLGLLNRAGFAKYGFKDGFRFKMQVKDNSTKTILERCFDGIYTIVASKLLYDQKKNRWKLNLSYSFDNKNISELDKEKILGVDVGVSCPLVASVFGDRDRFIIKGGEIEKFRKSVEARRRSMLEQTKYCGDGRIGHGRKKRTEPALNIGDKIARFRDTTNHKYSRALIEYAVKKGCGTIQMEKLTGITSKADRFLKDWTYYDLQTKIENKAKEVGINVVYIAPKYTSQRCSKCGYIHKDNRPNQAKFRCLKCDFESNADYNASQNIGIKNIDKTIKKERKKQKSEAQVNEK

SEQ ID NO: 104 - protein, 492 aa, unidentified
MAGKKKDKDVINKTLSVRIIRPRYSDDIEKEISDEKAKRKQDGKTGELDRAFFSELKSRNPDIITNDELFPLFTEIQKNLTEIYNKSISLLYMKLIVEEEGGSTASALSAGPYKECKARFNSYISLGLRQKIQSNFRRKELKGFQVSLPTAKSDRFPIPFCHQVENGKGGFKVYETGDDFIFEVPLIKYTATNKKSTSGKNYTKVQLNNPPVPMNVPLLLSTMRRRQTKKGMQWNKDEGTNAELRRVMSGEYKVSYAEIIRRTRFGKHDDWFVNFSIKFKNKTDELNQNVRGGIDIGVSNPLVCAVTNGLDRYIVANNDIMAFNERAMARRRTLLRKNRFKRSGHGAKNKLEPITVLTEKNERFRKSILQRWAREVAEFFKRTSASVVNMEDLSGITEREDFFSTKLRTTWNYRLMQTTIENKLKEYGIAVNYISPKYTSQTCHSCGKRNDYFTFSYRSENNYPPFECKECNKVKCNADFNAAKNIALKVVL

SEQ ID NO: 105 - protein, 487 aa, unidentified
MPDTDKGKRLTKVATFQIVKPVNMSWNEFGKMLHDVRYRYWRLANMAVCENYMRFYRWRTQQTDTNDHYKVKIINGILRKMLIEEKNADEKELSRYSRDGAVSGYVYGAFTQTKLSAITSKSKWGEVIKGKSALPLFKRDTSIPIMCTDKKPSMIEKTASGEYEVDLRICLKDKQLRPNGYPSVLLSTTKISDGQKAVLERLVSNKTNSLPGYRHRFFEVKEKRGDWYLSVSYDFPQAEATRLHPDIIVGVDLGWSVPLYAAINNGYARIGWRKLEPLAKSIKHLQKQTIVRRRSFQKGGKKDLAASTARTGHGIKRILQPIEKLEGKIDNAYKTLNHQLSHCIIEFAKNHGAGVIQIENLKGLAEELSGTFIGQNWRYHQLQEFIKYKAEEAGIAVKEVNPRYTSRRCSKCGYIHIGFDREYRDKNRKNGKSTMFECPECSKRIKDYKPLNADYNAAKNLATADIEEKIRLQCKEQGIEYKELPKD

SEQ ID NO: 106 - protein, 471 aa, unidentified
MPTITRKIKLELCTKGLSEEERKAQWNLLYHINDNLYRSANNISSKLYLDEHVSSLVWLKHKEHQTLKADLAKAKKQKIQDEKTIAELESRLKSCESEMSDQELAICKYTDEMSSKTLSYKFATELELNIYAQILTQVQSKVYADFQNDQKDVRDGKRAIRTYKKGMPIPFPWRNNIRMEPVKKGREYEFYIKWYNDIRFQLIFGKDRSNNRLILQRCFKLDENCVEDYQMRTSSIKMVKGANGTELFLYLVVDIPQEKHILNNKIVVGVDLGINVPAYVATNVTDDRKAIGDREHFLNTRMAISKRFHSFQRLKGTTGGRGKTKKLEPLERLKEKERNWVHTQNHLFSRDVITFALHVKAATIQMEDLSGYGKDDEGNVVEEKKFLLGKWSYYELQEMIKYKAKKVGMRVNFIKPAYSSQTCSWCGERGERNSTSFVCTNSECSHYGEDLHADYNAARNIARSKNIIRYE

SEQ ID NO: 107 - protein, 377 aa, unidentified
MIITRKIQILFAAQGEEFKKDKDTLYKWSNIVHHASNIVASNKYVCDHLQGMVYLTEEGKEAVSELSQKVDDIFNTSRMNTTYRMISSLYKGEIPTDILSCVNMQVSKLYNKERKKMADGDRSLRSYRSNIPIPFSANSLMRKWKYADKEYSFDLFGIPFKVVLGKDKSNNRSILERLMDGTYKAATSSIKIQNCEDETGKKTRKFFLLLCVEIPDKSYAGREDNILFAELSIDHPLLVSFPIKKEESKPIPIGNKQSYLYKRLQIQKGLDSCKASCKWNKGGRGRKRKMKSTERFKAKEHNFVDAYMHQISAALIKFAIKHDIGKLCLVDVDKKIKEAKESPFVLRNWSYYSLLTKIQYKAKMNGITVVMVDKNVL

SEQ ID NO: 108 - protein, 483 aa, unidentified
MPTITRKIRLHLCTDGLSEEERKAQWKMLYRINDNLYRAANNISSKLYLDEHISSMVRLKHAEYTSLKTELLKAKKADDEETVAELEARINVLNAELSAQEEAICSYATEMATRTLAGKFASELDLNIYGQILAEVKSVVFKNFNSDSKDVREGKRSIRTYKKGMPIPFPWNKTIRLEAVKKESSSKHDEDEYEVYLNWYKSSRTEKKAIRFRLDFGKDKSNNQQIVKRCLNLDNTSSESYQLQTSSIQMKKGSEGAELYLLLVVNIPQDQHVLNKKIVVGVDLGINVPAYVATNCTEERKSIGDREHFLNARIAFHRRFHSFQKLKGTTGGRGRKKKLEPLERLREKERNWVHTQNHLISRDVINFALQTKAATIQMEDLSGYGKDEEGNVKPENKFLQSRWSYFELQSMIKYKAAKCGIKVNLINPSYTSQTCSWCGQMGVRESTSFVCQNPECKKYGKDIHADYNAARNIARSNKTVKNE

SEQ ID NO: 109 - protein, 478 aa, unidentified
MPTITRKIELRLCTEGLSDEERKAQWMLLYHINDNLYRSANNISSKLYLDEHVSSMVRLKHAEYQSTAAELLKAKKNNADEGTISTLEDKVETLKTEMSAQGIAICNYATEMATRTLAGKFASELELNIYGQILAEVKNVVHTNFTNDAKEVREGKRSIRTYKKGMPIPFPWNKSIKIEPVKASSQNEGQDDYEFYLKWYNGLKFILHFGKDRSNNRQILKRCFGLDNLCNERYQMRTSSIQMKKGSNGMELYLLLVLSIPKEQHSLNKKVVVGVDLGINVPAYVATNCTEERRAIGDREQFLNTRMAIIRRKHSFQRLKGTAGGRGRKKKLDPLERLRETERNWVHTQNHLYSRDIIKFALETKAATIQMEKLKGFGRDDNGNVIEEKKFLLGKWSYYELQNMIKYKAGKVGIKVNFIAPAYTSQTCSCCGVRDDRNRKSTSFICHNPDCQMYGKEIHADYNAARNIARSKNVIKDE

SEQ ID NO: 110 - protein, 468 aa, unidentified
MPAITRKIELTLCTEGLSEEQRKEQWGLLYHINDNLYKAANNISSKLYLDEHVSSMVRMKHADYLSLLKELARAEKQKTPDDELIAELREKLSLAEQEMTDQELAICNYATEMATSTLAYNFAKEIETEIFGQILACLENNAHAVFVDDSPTVRRGERAIRNYKKGMPIPFPWNKSIRIVEKDGEFYLRWYNGMRFLLTFGKDRSNNRIIMKRCLKMDQDFEGEYKLCNSSIQMVKREGKTKLFLLIVVNIPQEHVELNKNIVVGVDLGVNVPAYVATNITEERKAIGDREHFLNTRMQFQRRYKSLQRLKGTAGGKGRTKKLEPLERLRKAEHNWVHTQNHLFSREVVNFAVQTRAATIHMEDLSGFGKDNDGNADEQKEFVLRNWSFYELQNMIAYKAAKYGIKVEKVKPAYTSKTCSWCGQLGFRQGVTFICENPACKQCGEKVHADYNAARNIANSKDIIKKNE

SEQ ID NO: 111 - protein, 478 aa, unidentified
MAGQRHTKVAKFQILKPAADMRWSELGRLLRDAQYRVYRLANLALSEKYLRFHLFRTGQTESLPECRIGRLNRQLRQMLKDEGGADDSVLDRFSRTGALPDTVVGALWQYRLHALTKGEKWNKVTRGETALPTFRRSMALPIRCDKRIHHRLERAALDSVELDLMICTRPYPRVILKTAKLDDGAAAILERLLDNEGQLLEGYRQRCFEVRYAEDEKAWWLHVTYDSPATPAPHLSKDIIVGVDLGFSCPMYVALSNGDARLGRRQFAALAARIRSLQTQVMARRRQMLSGGKASLSGDTARSGHGRKRKLLPIESLEGRINRAYTTLNHQLSISVVHFAVHHGAGVIQIENLEGLQNELTGTFLGQRWRYHQLQEFLNYKANEAGIEVRRVNPRYTSRRCSKCGYIHVDFNRAFRDAARQEGKVARFCCPKCEYEAHPDYNAARNLATVDIEGIIKVQCERQGIDRPSVENQDEVAK

SEQ ID NO: 112 - protein, 469 aa, unidentified
MPTITRKIELTLCTEGLSDQERKDQWNLLYHINDNLYRAANNISSKLYLDDHVGSMVRLKHAEYLSLLRALEKAKKQKAPDEEVIAELSQQVATAEQEMDEQAKAICQYATEMSTQTLSYRFATELETNIFGQILTCLRQGVFSTFNSDARDVKRGERSIRTYKKGMPIPFPWNDSLRIGFEDGEFYLRWYNGLRFRFDFGKDRSNNRLIVQRCMKMDKDYEGDYKLCNSSIQMVKREGKPKFFLLLVVNIPQERVELNKNIVVGVDLGINAPAYVATNTTPERKQIGDREHFLNERMAFQRRFKSLQRLKSTTGGRGRAKKLEPLERLRKAEQNWVHTQNHLFSREVIDFAVKARAATIHMEDLSGFGKDRDGNADERKEFVLRNWSYYELQNMITYKAAKYGIKVEKIRPAYTSKTCSWCGHQGFREGITFICENPECKKFGEKEHADYNAARNIANSKEIIKNNEE

SEQ ID NO: 113 - protein, 469 aa, unidentified
MPTITRKIELSLCTDGLSDEQLKEQRQLLYHINDNLYRAANNVSSKLYLDEHVSSMVRLKHADYLSLLRDLARAEKQKSPDEALISELRSKLAAAQREMTEQELAICRYATEMSTQSLSYRFVTEMETHIFAKILDCLKQGVYATFNSDARDVKRGERSIRNYKKGMPIPFAWSDSVRIEQEADEFYLRWYNGIRFRLVFGKDRSNNRLIVKRCLKLDKDYEGDYKLCNSSVQMVKREGKPKTFLLLVVKIPQEQVELNKKIVLGVDLGINYPVYAATNCTEERIYFGEREHFLNTRMQFQRRYKSLQRLKGTTGGKGRKKKLEPLERLRKAERNWVHTQNHLFSQKTVDFALQTHAATIHLEDLSGFGRDSDGSAEEKKEFVLRNWSYYELQQMITYKAAKYGIKVEKIRPAYTSQTCSWCGQRGFRQGVTFICENPECKKCGEKEQADYNAARNIAKSKDVIKDDDE

SEQ ID NO: 114 - protein, 453 aa, unidentified
MSIVTRKIELIPDIENLTHEESNQRCYKLLYNIDKKLYKLANLLVCQLFGLDNLLSLMRLQNDEYVKFQSKLASKSISKETQKNIKEHMKEIDKELLARKAEIAPKSPLTFAYRAIKGSLYAKDLPSDIFNTLKQDVFKHFNETKKEQLRGERSLATYKRGIPIPFSLMKKNVIVSEGDNYYLTWFEETRFKLNFGKDRSNNRAIIDNCLKTNKYKLCTAAKIQLKNKKLFFLVTVDIPETKNTIIKGKVMGVDLGVVHPAYVAVNDGPERSLIGDGDAFQKQRDVFRRRFKELQRCQLTQGGHGRKHKTKATEILRGKERNWVQTENHRISRKIVNLAIRWKVESIQMENLKGFGKDSEGEVETKHKRLLGRWSYFELQKDIEYKAQKAGIKVVYINPAYTSQTCHVCGKKGDRTERDTFICLNTECSCYGKPQDADMNAAINIARSKNIVK

SEQ ID NO: 115 - protein, 468 aa, unidentified
MPTITRKIELMLCSEGLSDEQRKEQWGLLYHINDNLYKAANNISSKLYLDEHVSSMVRMKHAEYLSLLKELARAEKQQTPDEGLIAELSRKLSAAEKEMADQELAICKYATEMSTQTLSYNFAKEIETNIFGQILTCLRQGVYATFNSDAKDVKRGERAIRNYKKGMPIPFPWNKSLKIEAEGGDFYLRWYNGLRFLLTFGKDRSNNRMIVKRCMKMDEDFEGEYKLCNSSIQLAKRDGKPKLFLLLVVNIPQEHVELNKKIVVGVDLGVNVPAYVATNITEERKAIGDREHFLNTRMAFQRRYKSLQRLKGTAGGKGRTKKLEPLERLRDAERNWVHTQNHLFSREVVNFAVQARAATIHMEDMSGFGKDKDGNADEKKEFVLRNWSFYELQNMIAYKSAKYGIKVVKIRPAYTSKTCSWCGQQGERKSTTFICENPECKHYGESIHADYNAARNIANSNDIVKENE

SEQ ID NO: 116 - protein, 385 aa, unidentified
MVITRKIEVFVCESDNDLRRSYYEKLYDIRNIAQEAANRATSMLYAIDNLIPCLDEDSRKLIQYIGAKGTPASRQNAAYTIMSHLYKDRMPGIMDMLSNLAQYVTKNYSEDRKRGMYKNALRSYKCSLPVPYQKKSFKGLRFNWYEDSDGDAHEGCFFSLAGVPLQMRFGRDRSNNRLIVERVISGEYKMCTSSLKFDGKKLFLLLCVDIPKQEANVDPKKTLYAYLGVMNPIICTCDVRAKQEYDSGYKCFEIGTKEEFNYRRRQIQEAVRRCQINNRYSSGGKGRKKKCQAIERWHEKEKNYVDTKLHTYSRMLVDLAVAHKCGTIVLLNQKKREDKAKDDNQNGEPFVLRNWSYYNLKDKIGYKCKLAGIKLVQDKEETEEE

SEQ ID NO: 117 - protein, 372 aa, unidentified
MVITRKIEVFVCEDSKDLRKEYYDKIYKCRDIAVKTANLGVSHLFMLDNTTPYLSDDDREKLTFLGCSGKKATKQNAPYVAASEKFKGQADMSMLSSVLQNVGKMYQDDKKKGMWSKSLRSYKANMPIPFKASCYRNLRFADYNDKEDKPHNGCFFTLMGIPFQCKFGKDRSGNRIIMQAVVDGKYKMCTSSLQIDGKKIFLLLCVDIPKKVVKLDESKTLYAFLGVMNPIVCTTDIKQKGDIDTDWKLWEIGTEAEFNYRRRQIQEAVKRCQVNNRYSRGGHGRFAKTKAIERWRAVERNYVDTKLHTYSKMLIDLAVKHKCGKIVLMNQLHREDAAKDDKFVLRNWSYHSLRTKIDYKAKMYGIKVEVEK

SEQ ID NO: 118 - protein, 483 aa, unidentified
MPVITRKIKLNLCTEGLSEDERKAQWKMLYRINDNLYRAANNISSKLYLDEHVSSMVRLKNAEYTSLVSDLMKAKKAEDEAAITDLEAKIESLKSEMTAQEEAICCYATEMATRTLAGKFASELDLDIYGQILAEVKSVVFKNFNSDSKEVREGNRSIRTYKKGMPIPFPWNKTIRLEAVKKELSGKHDEDEYDFYLNWYKSSRTDKKAIRFRLYFGKDKSNNQQIVKRCLHLDSTSSENYQMQTSSIQMKKGPEGAELYLLLVVNIPQEQHALNKKIVVGVDLGINVPAYVATNCTEERKAIGDRDHFLNTRMAFSRRFHSFQRLKGTSGGKGRKKKLEPLERLREKERNWVHTQNHLISRDVINFALQVKAATIQMEDLSGYGKDEEGNVKPENKFLQSKWSYFELQSMIKYKAAKCGIKVNLIAPAYTSQTCSWCGQMGIRESTSFVCQNPECKQYGKDIHADYNAARNIARSNKIVKNE

SEQ ID NO: 119 - protein, 477 aa, unidentified
MRISKTLSLRIVRPFYTPEVEAGIKAEKDKREAQGQTRSLDAKFFNELKKKHSEIILSSEFYSLLSEVQRQLTSIYNHAMSNLYHKIIVEGEKTSTSKALSNIGYDECKAIFPSYMALGLRQKIQSNFRRRDLKNFRMAVPTAKSDKFPIPIYRQVDGSKGGFKISENDGKDFIVELPLVDYVAEEVKTAKGRFTKINISKPPKIKNIPVILSTLRRRQSGQWFSDDGTNAEIRRVISGEYKVSWIEIVRRTRFGKHDDWFVNMVIKYDKPEEGLDSKVVGGIDVGVSSPLVCALNNSLDRYFVKSSDIIAFNKRAMARRRTLLRQNKYKRSGHGSKNKLEPITVLTEKNERFKKSIMQRWAKEVAEFFRGKGASVVRMEELSGLKEKDNFFSSYLRMYWNYGQLQQIIENKLKEYGIKVNYVSPKDTSKKCHSCTHINEFFTFEYRQKNNFPLFKCEKCGVECSADYNAAKNMAIA

SEQ ID NO: 120 - protein, 478 aa, unidentified
MEEKTKRLQKVAKFQIVKPVNMTWVELGKMLRDVRYRLWRLANMAVCENYMRFYQWRIGKTDANENHKVKILNRRLREMIIEEKQADAKELMRYSRDGVVSGYICGAFEKIHLSAIKNKSKWREVIRGKSNLPLFKRDLPIPINCSDHKPSLIAKTESDEYEVDLRICQKPYPRVLLSTAKISGGERAILERLVSNKTNSLPGYRHRFFEIKEKPKGRWNLHVTYDFARSEATMLHSDIIVGVDLGWSVPLYAAVNKGHARIGWRKLEPLAKRIRHLQKQVKARRLSVQKGGQRDLAAPTARAGHGRKRILQPIEKLEGKIDDAYKTLNHQLSHCVIEFAKNNGAGVIQVENLEGLKDTLTGTFIGQNWRYNQLQNYIEYKAKEAGMELKKVNPCQTSQRCSNCGFIHRDFTFEYRQANKKNGKAAMFECPECSKKENYKPLNADYNAARNLATAGIEGKIRLQCEKQGIEYKGLPEE

SEQ ID NO: 121 - protein, 453 aa, unidentified
MSKITRKIEIIPDIEGLTHDESNKKCYGAFYTFDKNLYKVANLLVSQLYGLDNLLSLMRLQNDEYVKCQSKLSLKSTTDAEKENLKKRMKKIDAELVSIKNGMAPKHPQTFAYRAVTNCVYAKNIPSDILNTLKQDVYKHFNDTKKEQFLGERSLTTYKRGMPVPFSIEKKHAIVCDGDNYYLPWFEDTRFRLNFGRDKSNNRAIIDNCIKTKRYKLCAAAKIQLKDKKLFLLVTVDIPATETTSVKGKVMGVDLGVVNPAYVAVNDGPERSRIGNGEAFQKQRDVFRRRFRELQRSQLTQGGHGRKHKTKATETLRGKERNWVQTENHRISREIVNLASRWKVECIQMESLKGYGKNQEGEVEDNHKRLLGRWSYFELQKDIEYKAAMVGIQVKYINPAYTSQTCHVCGQRGNRIERDTFICTNPECTCYNQAQDADMNAAINIAKSKDVVK

SEQ ID NO: 122 - protein, 469 aa, unidentified
MPTITRKIEMKLCTEGLSDQERKDQWNLLYHINDNLYRAANNISSKLYLDDHVLSMVRLKHAEYLGLLRALEKAKKQKIPDEEVIAELSQKVAAAEQEMDDQAKAICQYATEMSTQSLSYRFATELETGIFTKILDCLKQGVFATFNSDTRDVKRGERSIRTYKKGMPIPFAWNDSLRIELEDGEFYLRWYNGLRFRFDFGKDRSNNRLIVRRCLNMDEDYEGDYKLCNSSIQMVKREGLAKFFLLMVVNIPQEQVELNKKIVVGVDLGINAPAYVATNITSERKQIGDREHFLNERMAFQRRFKSLQRLKGTTGGRGRAKKLEPLERLRKAEQNWVHTQNHLFSREVIDFAVKSRAATIHMEDLSGFGKDRDGNADDKKEFVLRNWSYYELQSMITYKAAKYGIKVEKIRPAYTSKTCSWCGHQGFREGITFICENPECKKYGEKEHADYNAARNIANSIEIVKNNEE

SEQ ID NO: 123 - protein, 478 aa, unidentified
MKDYIRKTLSLRILRPYYGEEIEKEIAAAKKKSQAEGGDGALDNKFWDRLKAEHPEIISSREFYDLLDAIQRETTLYYNRAISKLYHSLIVEREQVSTAKALSAGPYHEFREKFNAYISLGLREKIQSNFRRKELARYQVALPTAKSDTFPIPIYKGFDKNGKGGFKVREIENGDFVIDLPLMAYHRVGGKAGREYIELDRPPAVLNVPVILSTSRRRANKTWFRDEGTDAEIRRVMAGEYKVSWVEILQRKRFGKPYGGWYVNFTIKYQPRDYGLDPKVKGGIDIGLSSPLVCAVTNSLARLTIRDNDLVAFNRKAMARRRTLLRQNRYKRSGHGSANKLKPIEALTEKNELYRKAIMRRWAREAADFFRQHRAATVNMEDLTGIKDREDYFSQMLRCYWNYSQLQTMLENKLKEYGIAVKYIEPKDTSKTCHSCGHVNEYFDFNYRSAHKFPMFKCEKCGVECGADYNAARNIAQA

SEQ ID NO: 124 - protein, 480 aa, unidentified
MPFKVLKLKIIKPVNMDWNELGQSIRDTRYRVYRLANLAVSEAYLAFHLWRAGKTDAIPKATAGQLNRRLRDMLLEEARTKAVKDRKNTGEKGTEDDAKKAQKEMNKFSKTGALPDTVAGALFMYKVKGLISKGKWTQVIRGKSALPTFRNNMAIPIRCDKKTQRRLERTENGVELELMIRNKPYPRVLLGTQGIGEGAEAIIERLLSNESQAEQGYKQRYFEVREDVNRTWWLYVCYALPASTPPRLDPSKIVGVDLGFTCPMYAAISNGHARLGYRAFSSLAARVKALKLRTMRRRREIQRGGRTIVSGEAARSGHGRKRKLLGIEKLQGRVNQAYTTLNHQMSAAVVKFAIENGAGTIQVENLEGLREELSGTFLGQMWRYFQLQEFLQYKAEENGIVIRKVNPRYTSRRCSQCGHINKEFTRKARDRNAEGGYSAKFKCPDCEYEADADYNAAKNLAVDGIEGIIEKQCGSQGIVL

SEQ ID NO: 125 - protein, 494 aa, unidentified
MFLYKELKTMAKTNAEEGKIENKEKRLTKVAKFQIVKPVNMTWPEFGKMLGDVRYRLSRVANMAVTEKYLESQQKRTGQKIQRENTLVTIANRKLREMLKKEKVKEEELDRYSRDGAVSGYVTGPFEHNKLSAISKRFKEVLKGNMSLPNFKREMAIPINCSNAKLSTIEKTETGEYVVDLRISQKPWPRVLLSTNRISNGQREILERLAANKTFSDDGYKHLFFEVKQQGKDWFLSVTYSFPKSEAPKLHKDIIVGVDLGWSVPLYAAVNKGYARIGWQKFRPLAERIKHLQKQVKARRITIQKGGQQDLATPTARTGHGRKRILRPIEKLERKIENAYTTLNHQLSHCVIEFAKNNGAGVIQIENLSGLANELSGTYIGQNWRYEQLQEYIRYKAEEAGIEVKHVNPCRTSQRCSECGFINDKFNFEYRQANRNNGMSAMFECPECKKNKKDYKPINADYNAAKNLTTANIDEIIRLQCKKQGIEYKELPKD

SEQ ID NO: 126 - protein, 453 aa, unidentified
MSKITRKIELIPDIENLTHEESNQRCYKVFYNIDNKLYKVANLLVCQLFGLDNLLSLMRLQNDEYVKCQSKLASKSISEETKRDIKKRMEAIDKELLARKDEIAPKHPQTFAYRAIKDSDYAKDLPSDIFNTLKQDVFKHFNETKKEQLRGERSLTTYKRGIPVPFNLMKKNVIVSDGDNYYLTWFEETRFKLNFGKDRSNNRAIIDNCLKTNKYKLCTAAKIQLKNKKLFLLVTVDIPETKNKIIKGKVMGVDLGVVHPAYVAVNDGPERSLIGDGDAFQKQRDVFRRRFRELQRCQLTQGGHGRKHKTKATENLRGKERNWVQTENHRISREIVNLAIRWRVETIQMENLKGFGKDSDGDVETKHQRLLGRWSYFELQKDIEYKAAMAGIKVVYVNPAYTSQTCHVCGERGDRTERDTFICTNTECDCYGKPQDADMNAAINIARSKNIVK

SEQ ID NO: 127 - protein, 427 aa, unidentified
MTKVVKLPLICEQSDKDGNPIDYKKIYEILFELQRQTREIKNKSIQYCWEFSNFSSDYYKQNHEYPKEKDILSYTLVGFVNDKFKTGNDLYSGNCSTTVRGACGEFKNSKTDFLKGTKSIINYKGNQPLDLHNKTIRFECIGKDYYAYLKLLNRPAFQRNNFSSSEIKFKVLVYDNSSKTIVERCIDNIYKISASKLIYNEKKKCWVLNLSYSFTNNNVCELDENKILGVDLGIHYPICASVNGERKFFKIDGGEIDHTRRKIEVRKKSLLKQGSSCGEGRIGHGIKTRNKPVYNIEDKIACFRDTANHKYSRALINYAVNNNCGIIQMEKLTGITADSDRFLKNWSYFDLQTKIEYKAKEAGITVVYIDPQYTSQRCSKCGYISKENRKVQAKFCCQKCGYEANADYNASQNIGIKDIDKIIKNTK

SEQ ID NO: 128 - protein, 472 aa, unidentified
VPITKTISLRILRPYYPPEIEAKIKAEKEKRKENGDTGSLNSSYYRELKKEYPSIIINDEFFPLLSEMQRNITSIYNRTISHLYHRLIIKKESISTAKALSEGPYRDFKSTFNSYIALGLRQKVQSNFRKKDLMAFKIALPTAKSDKFPIPIYMQTNFKIKESPDSDFIIELPLVEYIAKETKGKNKMFTKVEILSPPKVKNIPVILSTRRRKESGQWFSDEGTNAEIRRIISGEYKVSWIEIVKRTRFGKHDWFVNMVISFEESQEGLDPDVIGGIDIGVSKPLICAINNSLDRYIVKGDDIIAFNRRALSRRRSLLRRNRLKRSGHGSRNKLEPITVLTEKNERFKKSIMQRWAKEVAEFFKSKRASIVQMEELTGIKEREDFFSKTLRMYWNYGQLQKTVENKLREYGIEVRYASPKDTSRRCHSCGHINDYFTFEFRQQNNFPLFKCMNCGIECSADYNAARNIAIAR

SEQ ID NO: 129 - protein, 1331 aa, unidentified
MNRIYQGRVSKVEIPDGKDEWKKLDDGESALWQHHQLFQDDVNYLLAAFAALVPTSCEDDIWKDYQAAIERSWESYTGRQGIWDRPFENACVIVGCKKDASFKEFRRKLNSLTGSKASEKQKFEALKQLFEPATEAAKKLKKHDEPVEESLKGKAKDLFGSTLVNLCAQKTKVTPRDVIAKQRNRASECTKKVNEGERLKWADVFYFKTDTSAAKWSREDAAKNIIQFLDKLLGEVEEKEKDAKTSDQKKKMADLAERLEKQKKPLAAWCNNSKTDLPTTEPTRKGSGGYDLKAAVLFSLQPDLDGFRDAFLLFNQARLKEEFATTEKGDAAYIARMAGGVARPVFPFFCDVWAGKVNDEKIGQGIWPDFEKQAFSEVFTKIGQFIVRGRKFELRLAIADQIIAKIETQKKSDARLQAVERIAEDLADELPDTAVDENGQKRPYGIRERTLKGWRKVRPAWREALKKTPNLTAEDLIKQKNRMQERQREKYGSASLFDRLAKEPEIWNHDDKEDAVETWADYVENLEEKAHLETERLFAPAHATLSPRFFRWSETNNKEHLEASSPDVPFELKADALDLSKKEKSQIKIHFWSPRLWRDGLRGKKENLDKDEPDQNWMPPVLRAFVKARKWPCDKQSFAGASVRLAPRCKENIQLVFEPELHTEILSAKWKENFPFSPAKNKESESVGLFWPRTKEDKVLWFDKGETRCLGVDLGLTNSAAWQILQATNKDATAKAPRLRHRLNPDSEKAAWFAHSITNGIVRVAGEDCWGWRKFAPDEKAKLRAELKKPAGKRNALCRKFLSLNREIEFETATHSFLPELSGSGGRNPTDDETKEAAEFFSTLKTKGFDITDRQPSWGKNLSFPKQNDELLWGLKRVRAQLFRLNRWSEQLGKERDSKPYQSAIEIIGNLRSDDPLIELATLKSEPKRLKSRIAELAGEYLDCFKTLLPRIADRILPWRRGHWSWKPCDNDWHRMELDASKPRPEALLAGQRGISLPRLNQLKDLRQLAQSLNHLCRRKQIKRNETVPEPFEDCRQAMEDAREDRAKKIAHEVFAIALGVELAPPPPDKQERKQTESLHGVYRCLERGPVNFIALENLGGYNPSAKQGRRENRQLSSWLKGRIHKILGELCEMVGMPIVLVNAEYTSRFSAKDHSPGFRAEEVQTDDSRRSFWQRKAKEEPSGWQNEFLCWLNKVPDGKSLLLPKKGGEFFVPLGEGTSLYHADLNAAYRIALRALAHRDRAELLGQTWIEKKPYLVDVAGVFPDSILRNGCAFKTISSSERLWEKVNGDLAMQRCREINLARFASWKIALPQQIISEALPPDEEDDIPM

SEQ ID NO: 130 - protein, 517 aa, unidentified
MSEATKTLAYRYRLRLTPAQEDILDRSQEQLRLVWNHLVRSQHKVEHEWRHGRAASIKNELLELSLAKNATGQAIPSARKITEERGVSMEEALRLMRQKFVEKVSAIPLRKKDGSRCLRIARRKMATEYAVTVVNAKFKHYYGLGARMCKVLRDKFQKCSDMWIKGKFRRPRFKRKGESVALQRQVQSNSPFKLKRFSDLSALGGQALKKCEVIIHRPLPDSAEIKQIAVSGRRGQRHLIVMFKAASSDVAKNFPATNRTAGVDPGIKVALTITPLDSPDFGTSDKIEKQPDLARDACFLKRLRRLQRKHDRQRRQNNPECFDEKGRWIKGKRLHNESKNMQRTQSRITAMNTHLAESRRDFYHNAACEILRSFDNVAVGKWRPAQTRQRKPTTPSPKGLGAARRATNRISYDHAISLFISYLKDKAERSVTTKHVQEVSEFGSTRSCPKCGKLTGPVGTEGLAVRDWTCVNCNTTFQRDAASAWQIAKRFKAEVASTSQPAESQDSANSASVLTQV

SEQ ID NO: 131 - protein, 470 aa, unidentified
MPTLTRKVELYVVGDKEEVSRVYDYIRLAMNATYKCFNECMTALYIAQVKEDTKEDRKELNHLYSRQTYTKKETAFTNDIVFPEGLALAAYVNRMAQQKFVTSLKNGLMYGCVSLPTFKKDCAVPLHVKFVSLAGEKGTNTGFYHEYADVNDLVNALEYDNSPKVFLRFPNNITFGVVFGNPYRGREQRSVFSKIFLGEYKIQGSSIQINSRGKIILNLSMEVPKKKMEHIEGRVVGVDVGLAIPAMCAINDDDYTRSAIGNIDDFLKVRTQIQSQRRRLQKSLKNTSSGHGRTKKLKPLERIAEKERNFANTYNHMVSKRVVDFAVKNGASQINIEDLSGFAKDKNGKSVEDDNMKRVLSNWSYFELQQQIRYKAEQYDIKVRTVNPAYTSQTCSYCGQIGKRETQSKFVCTNPDCKCHKMYKKDWFNADFNAARNIALSTDYTDDEDGKKTKKKKSAKKKPEKKTEEA

SEQ ID NO: 132 - protein, 500 aa, unidentified
MSGASGQITRDNKAQRSGPNKGEMSEDHSSTKRPKRVVKVAKYRIIKPVGEMTWPELGEILRTVRYRVFRLGNLAVSEAYLNFHAFRTGKAEEFKSETIGKLSRRLRDMLISEGVKKEDIDRYSATGAVPDTVAGALGQYKVRGITSPAKWRQVIRGTVSLPTFRNDMAIPVRCDKPAQRRLEKAKSEEVEVDLMICRKPYPRVLIGTADLGGGQQAILERLLDNKDNSSDGYRQRLFEIKQDTQSKKWFLFVTYDFPSSGALPLDPNVAVGVDLGVSVPLYAAINNGHARLGRRQFQALGSRIRSLQTQVDARRRAIQRGGRSDVSQSTARSGHGVRRKLQPTEKLRKRIDRSYSTLNHQLSAAVVEFAKNQGAGTVQMEDLGGLREELTGTFIGARWRYHQLQQFLEYKCDEAGITLNKVNPMYTSRRCSECGFIDKDFDRAFRDRSRSDGRVARFICPECSYEADPDYNAARNIATLDIDKLIRVQCQKQGLKYDAL

SEQ ID NO: 133 - protein, 459 aa, unidentified
MIITRKIELWLSEDDNELRKAKWSYLKELNDEVYRAANFIVNNQYFNEILENRVIMQDTRLIDIDSEIRKLYKSREKNKEKIDELKKIKKIRYQEAKNFYQTSKQNVTYQLTSREFPNIPANIVTSLNASIIKTLKTEWNEIKSGKRAVRNYRKGMPIPFNFSSSQKWFENKGEDIFLNWLGGLKFKLFFGRDKSNNRAIVERAINKEYKYADSSIQLKDKKIFLLFVVDIPYEKANLNKNIAAGVDLGIAFPAFCALSEGYSRLSIGNKEDLLKVRLQMQSRRKRLQKALKITSGGKGRTKKLKALESLTNKEKNYVTTYNHKVSYQVIKFAKDNKAGIIKLEFLEGFGEDEKNKFILRNWSFYQLQKMIEYKAKREGIEVLYIDPYHTSQTCAICGNYEEGQREKQEDFICKNPECKNFEKIVNADYNAALNIAKSNKIVSSSEQCEYNKKHENNVL

SEQ ID NO: 134 - protein, 529 aa, unidentified
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 135 - RNA, 36 nt, synthetic construct
  source 1..36: mol_type = other RNA
ggagagatctcaaacgattgctcgattagtcgagac

SEQ ID NO: 136 - RNA, 36 nt, synthetic construct
  source 1..36: mol_type = other RNA
gtcggaacgctcaacgattgcccctcacgaggggac

SEQ ID NO: 137 - RNA, 36 nt, synthetic construct
  source 1..36: mol_type = other RNA
accaaaacgactattgattgcccagtacgctgggac

SEQ ID NO: 138 - RNA, 36 nt, synthetic construct
  source 1..36: mol_type = other RNA
ggatctgaggatcattattgctcgttacgacgagac

SEQ ID NO: 139 - RNA, 36 nt, synthetic construct
  source 1..36: mol_type = other RNA
gtctcagcgtactgagcaatcaaaaggtttcgcagg

SEQ ID NO: 140 - RNA, 36 nt, synthetic construct
  source 1..36: mol_type = other RNA
ctttcaagactaatagattgctccttacgaggagac

SEQ ID NO: 141 - RNA, 20 nt, synthetic construct
  source 1..20: mol_type = other RNA
attgctccttacgaggagac

SEQ ID NO: 142 - RNA, 36 nt, synthetic construct
  source 1..36: mol_type = other RNA
gtagaagacctcgctgattgctcggtgcgccgagac

SEQ ID NO: 143 - RNA, 37 nt, synthetic construct
  source 1..37: mol_type = other RNA
ggatccaatcctttttgattgcccaattcgttgggac

SEQ ID NO: 144 - RNA, 36 nt, synthetic construct; identical to SEQ ID NO: 140
  source 1..36: mol_type = other RNA

SEQ ID NO: 145 - RNA, 20 nt, synthetic construct
  source 1..20: mol_type = other RNA
attgctccttacgaggagac

SEQ ID NO: 146 - RNA, 36 nt, synthetic construct; identical to SEQ ID NO: 139
  source 1..36: mol_type = other RNA

SEQ ID NO: 147 - RNA, 36 nt, synthetic construct
  source 1..36: mol_type = other RNA
atggcaacagactctcattgcgcggtacgccgcgac

SEQ ID NO: 148 - RNA, 36 nt, synthetic construct; identical to SEQ ID NO: 137
  source 1..36: mol_type = other RNA

SEQ ID NO: 149 - RNA, 34 nt, synthetic construct
  source 1..34: mol_type = other RNA
gtcgctctctaacgcttgcccagtacgctgggac

SEQ ID NO: 150 - RNA, 37 nt, synthetic construct
  source 1..37: mol_type = other RNA
ggttgaaccctcaacagattgctcggtaagccgagac

SEQ ID NO: 151 - RNA, 37 nt, synthetic construct; identical to SEQ ID NO: 150
  source 1..37: mol_type = other RNA

SEQ ID NO: 152 - RNA, 36 nt, synthetic construct
  source 1..36: mol_type = other RNA
cttgaaatcctgtcagattgctcccttcggggagac

SEQ ID NO: 153 - RNA, 36 nt, synthetic construct
  source 1..36: mol_type = other RNA
gctggaagactcaatgatggctccttacgaggagac

SEQ ID NO: 154 - RNA, 36 nt, synthetic construct; identical to SEQ ID NO: 153
  source 1..36: mol_type = other RNA

SEQ ID NO: 155 - RNA, 36 nt, synthetic construct
  source 1..36: mol_type = other RNA
ctaggaacgcacgcagattgctcggtacgccgagac

SEQ ID NO: 156 - RNA, 36 nt, synthetic construct
  source 1..36: mol_type = other RNA
attgcaacgcctaaagattgctcgatacgtcgagac

SEQ ID NO: 157 - RNA, 36 nt, synthetic construct
  source 1..36: mol_type = other RNA
gttcggcraycctttgattgctcagtacgctgagac

SEQ ID NO: 158 - RNA, 36 nt, synthetic construct
  source 1..36: mol_type = other RNA
gttgaacctagatcagatggctcagtacgctgagac

SEQ ID NO: 159 - RNA, 36 nt, synthetic construct
  source 1..36: mol_type = other RNA
ccctcaacacgtcagaaatgcccggcacgccgggac

SEQ ID NO: 160 - RNA, 36 nt, synthetic construct
  source 1..36: mol_type = other RNA
gtcgcaagactcgaataattgcccctctatggggac

SEQ ID NO: 161 - RNA, 37 nt, synthetic construct
  source 1..37: mol_type = other RNA
gctggggaccgatcctgattgctcgctgcggcgagac

SEQ ID NO: 162 - RNA, 36 nt, synthetic construct
  source 1..36: mol_type = other RNA
ctctcaatggataacgattgctctctacggagagac

SEQ ID NO: 163 - RNA, 36 nt, synthetic construct
  source 1..36: mol_type = other RNA
gttgaaccctcaacagattgctcggtaagccgagac

SEQ ID NO: 164 - RNA, 36 nt, synthetic construct
  source 1..36: mol_type = other RNA
actgaaaccaccaacgattgcgctcctcggagcgac

SEQ ID NO: 165 - RNA, 33 nt, synthetic construct
  source 1..33: mol_type = other RNA
cgttgcagctcgcacgttggcactggttgaagg

SEQ ID NO: 166 - RNA, 34 nt, synthetic construct
  source 1..34: mol_type = other RNA
cgttgcagctcgcacgttggcactgggttgaagg

SEQ ID NO: 167 - RNA, 17 nt, synthetic construct
  source 1..17: mol_type = other RNA
ttggcactggttgaagg

SEQ ID NO: 168 - RNA, 13 nt, synthetic construct
  source 1..13: mol_type = other RNA
cactggttgaagg

SEQ ID NO: 169 - RNA, 32 nt, synthetic construct
  source 1..32: mol_type = other RNA
gttgcaactcacgcgcgtatgtggcttgaagg

SEQ ID NO: 170 - RNA, 32 nt, synthetic construct
  source 1..32: mol_type = other RNA
gttgcaattcatatctccgggtggattgaagg

SEQ ID NO: 171 - RNA, 32 nt, synthetic construct
  source 1..32: mol_type = other RNA
gttgcagcgtgcgcgagcgtgtggcttgaagg

SEQ ID NO: 172 - RNA, 17 nt, synthetic construct
  source 1..17: mol_type = other RNA
agcgtgtggcttgaagg

SEQ ID NO: 173 - RNA, 13 nt, synthetic construct
  source 1..13: mol_type = other RNA
tgtggcttgaagg

SEQ ID NO: 174 - RNA, 32 nt, synthetic construct
  source 1..32: mol_type = other RNA
gttgcaactcgcacgtgaatgcgacttgaagg

SEQ ID NO: 175 - RNA, 17 nt, synthetic construct
  source 1..17: mol_type = other RNA
tgaatgcgacttgaagg

SEQ ID NO: 176 - RNA, 32 nt, synthetic construct
  source 1..32: mol_type = other RNA
gatgcaactcgtgtgtatgtgcgagttgaagg

SEQ ID NO: 177 - RNA, 32 nt, synthetic construct
  source 1..32: mol_type = other RNA
gacgcaactcgcgcgcgggcatgtattgaggg

SEQ ID NO: 178 - RNA, 32 nt, synthetic construct
  source 1..32: mol_type = other RNA
gatgcatctgacacagctgggtgagttgaagg

SEQ ID NO: 179 - RNA, 17 nt, synthetic construct
  source 1..17: mol_type = other RNA
gctgggtgagttgaagg

SEQ ID NO: 180 - RNA, 32 nt, synthetic construct
  source 1..32: mol_type = other RNA
gttgcaacacatgtatgtgggtgagttgaagg

SEQ ID NO: 181 - RNA, 32 nt, synthetic construct; identical to SEQ ID NO: 171
  source 1..32: mol_type = other RNA

SEQ ID NO: 182 - RNA, 32 nt, synthetic construct
  source 1..32: mol_type = other RNA
gttgcaatttgtatacgagtgtgacttgaagg

SEQ ID NO: 183 - RNA, 32 nt, synthetic construct
  source 1..32: mol_type = other RNA
gctgcaacacgcgcgggtacgcgggttgaagg

SEQ ID NO: 184 - RNA, 32 nt, synthetic construct
  source 1..32: mol_type = other RNA
gttgcaactcgcacgttggcactgattgaagg

SEQ ID NO: 185 - RNA, 36 nt, synthetic construct
  source 1..36: mol_type = other RNA
gctgtagccctgctcaaattgtagggcgcatgcagg

SEQ ID NO: 186 - RNA, 36 nt, synthetic construct
  source 1..36: mol_type = other RNA
gttgtagtcgacctgaatctgtggggtgcttacagg

SEQ ID NO: 187 - RNA, 17 nt, synthetic construct
  source 1..17: mol_type = other RNA
tgtggggtgcttacagg

SEQ ID NO: 188 - RNA, 36 nt, synthetic construct; identical to SEQ ID NO: 186
  source 1..36: mol_type = other RNA

SEQ ID NO: 189 - RNA, 36 nt, synthetic construct
  source 1..36: mol_type = other RNA
ggtgtatgtaaccgcaatttgaagggtgcatacagg

SEQ ID NO: 190 - RNA, 36 nt, synthetic construct; identical to SEQ ID NO: 186
  source 1..36: mol_type = other RNA

SEQ ID NO: 191 - RNA, 17 nt, synthetic construct
  source 1..17: mol_type = other RNA
tgtggggtgcttacagg

SEQ ID NO: 192 - RNA, 34 nt, synthetic construct
  source 1..34: mol_type = other RNA
gttggaatcgaccttaatttgaggtgtgcttaca

SEQ ID NO: 193 - RNA, 36 nt, synthetic construct
  source 1..36: mol_type = other RNA
gctgaaagagcagagaatttgttgtgtgcatacagg

SEQ ID NO: 194 - RNA, 36 nt, synthetic construct
  source 1..36: mol_type = other RNA
gttggagtcggcttgaatctgcggggtgcttacagg

SEQ ID NO: 195 - RNA, 17 nt, synthetic construct
  source 1..17: mol_type = other RNA
tgtggggtgcttacagg

SEQ ID NO: 196 - RNA, 36 nt, synthetic construct
  source 1..36: mol_type = other RNA
gttggaatcgaccttaatttgaggtgtgcttacagg

SEQ ID NO: 197 - RNA, 36 nt, synthetic construct
  source 1..36: mol_type = other RNA
gttgtaagagacccgaattttagctgtgtatacagg

SEQ ID NO: 198 - RNA, 36 nt, synthetic construct
  source 1..36: mol_type = other RNA
gttgtgaatgcaggcatttttgatggtaaatccaac

SEQ ID NO: 199 - RNA, 13 nt, synthetic construct
  source 1..13: mol_type = other RNA
tggtacatccaac

SEQ ID NO: 200 - RNA, 36 nt, synthetic construct
  source 1..36: mol_type = other RNA
actgtcagacaatgcaaaatgtgtggtacatccaac

SEQ ID NO: 201 - RNA, 13 nt, synthetic construct
  source 1..13: mol_type = other RNA
tggtacatccaac

SEQ ID NO: 202 - RNA, 10 nt, synthetic construct
  source 1..10: mol_type = other RNA
tggtacatcc

SEQ ID NO: 203 - RNA, 65 nt, synthetic construct
  source 1..65: mol_type = other RNA
tggggcagttggttgcccttagcctgaggcatttattgcactcgggaagtaccattacatccaac

SEQ ID NO: 204 - RNA, 23 nt, synthetic construct
  source 1..23: mol_type = other RNA
tggtacatccaactctaggcgcc

SEQ ID NO: 205 - RNA, 15 nt, synthetic construct
  source 1..15: mol_type = other RNA
aatggtacatccaac

SEQ ID NO: 206 - RNA, 20 nt, synthetic construct
  source 1..20: mol_type = other RNA
tggtacatccaactctaggc

SEQ ID NO: 207 - RNA, 22 nt, synthetic construct
  source 1..22: mol_type = other RNA
tggtacatccaactctaggcgc

SEQ ID NO: 208 - RNA, 21 nt, synthetic construct
  source 1..21: mol_type = other RNA
tggtacatccaactctaggcg

SEQ ID NO: 209 - RNA, 19 nt, synthetic construct
  source 1..19: mol_type = other RNA
tggtacatccaactctagg

SEQ ID NO: 210 - RNA, 16 nt, synthetic construct
  source 1..16: mol_type = other RNA
aaatggtacatccaac

SEQ ID NO: 211 - RNA, 16 nt, synthetic construct
  source 1..16: mol_type = other RNA
tggtacatccaactct

SEQ ID NO: 212 - RNA, 15 nt, synthetic construct
  source 1..15: mol_type = other RNA
tggtacatccaactc

SEQ ID NO: 213 - RNA, 14 nt, synthetic construct
  source 1..14: mol_type = other RNA
tggtacatccaact

SEQ ID NO: 214 - RNA, 18 nt, synthetic construct
  source 1..18: mol_type = other RNA
tggtacatccaactctag

SEQ ID NO: 215 - RNA, 13 nt, synthetic construct
  source 1..13: mol_type = other RNA
tggtatatccaac

SEQ ID NO: 216 - RNA, 17 nt, synthetic construct
  source 1..17: mol_type = other RNA
tggtacatccaactcta

SEQ ID NO: 217 - RNA, 14 nt, synthetic construct
  source 1..14: mol_type = other RNA
atggtacatccaac

SEQ ID NO: 218 - RNA, 12 nt, synthetic construct
  source 1..12: mol_type = other RNA
tggtacatccaa

SEQ ID NO: 219 - RNA, 11 nt, synthetic construct
  source 1..11: mol_type = other RNA
tggtacatcca

SEQ ID NO: 220 - RNA, 36 nt, synthetic construct
  source 1..36: mol_type = other RNA
gctgtcagtagtagtaaaaatgggggtacatccaac

SEQ ID NO: 221 - RNA, 13 nt, synthetic construct
  source 1..13: mol_type = other RNA
tggtacatccaac

SEQ ID NO: 222 - RNA, 36 nt, synthetic construct
  source 1..36: mol_type = other RNA
actgtcagtacatgcaaaaatgagggtacatccaac

SEQ ID NO: 223 - RNA, 13 nt, synthetic construct
  source 1..13: mol_type = other RNA
tggtacatccaac

SEQ ID NO: 224 - RNA, 36 nt, synthetic construct
  source 1..36: mol_type = other RNA
actgtcagacaatgcaaaatgagtggtacatccaac

SEQ ID NO: 225 - RNA, 13 nt, synthetic construct
  source 1..13: mol_type = other RNA
tggtacatccaac

SEQ ID NO: 226 - RNA, 36 nt, synthetic construct
  source 1..36: mol_type = other RNA
gctgttagaacatacaaaatgaaaggtacatccaac

SEQ ID NO: 227 - RNA, 36 nt, synthetic construct
  source 1..36: mol_type = other RNA
gctgcatgtcatggcaaaaggaaaggtacatccaac

SEQ ID NO: 228 - RNA, 13 nt, synthetic construct
  source 1..13: mol_type = other RNA
tggtacatccaac

SEQ ID NO: 229 - RNA, 36 nt, synthetic construct
  source 1..36: mol_type = other RNA
gctgtcagacacctaaaaaatgagggtacatccaac

SEQ ID NO: 230 - RNA, 13 nt, synthetic construct
  source 1..13: mol_type = other RNA
tggtacatccaac

SEQ ID NO: 231 - RNA, 36 nt, synthetic construct
  source 1..36: mol_type = other RNA
gctgtgagtcacagtaaaaatgaaggtatatccaac

SEQ ID NO: 232 - RNA, 13 nt, synthetic construct
  source 1..13: mol_type = other RNA
tggtacatccaac

SEQ ID NO: 233 - RNA, 36 nt, synthetic construct; identical to SEQ ID NO: 222
  source 1..36: mol_type = other RNA

SEQ ID NO: 234 - RNA, 13 nt, synthetic construct
  source 1..13: mol_type = other RNA
tggtacatccaac

SEQ ID NO: 235 - RNA, 36 nt, synthetic construct; identical to SEQ ID NO: 231
  source 1..36: mol_type = other RNA

SEQ ID NO: 236 - RNA, 13 nt, synthetic construct
  source 1..13: mol_type = other RNA
tggtacatccaac

SEQ ID NO: 237 - RNA, 36 nt, synthetic construct
  source 1..36: mol_type = other RNA
gatgtatatgctatgattttgtatggtacatccaac

SEQ ID NO: 238 - RNA, 13 nt, synthetic construct
  source 1..13: mol_type = other RNA
tggtacatccaac

SEQ ID NO: 239 - RNA, 36 nt, synthetic construct; identical to SEQ ID NO: 220
  source 1..36: mol_type = other RNA

SEQ ID NO: 240 - RNA, 37 nt, synthetic construct
  source 1..37: mol_type = other RNA
gttgcagaacccgaatagacgaatgaaggaatgcaac

SEQ ID NO: 241 - RNA, 13 nt, synthetic construct
  source 1..13: mol_type = other RNA
tggtacatccaac

SEQ ID NO: 242 - RNA, 32 nt, synthetic construct
  source 1..32: mol_type = other RNA
gttgagctctgcattacgcagatgaatgacga

SEQ ID NO: 243 - RNA, 32 nt, synthetic construct
  source 1..32: mol_type = other RNA
gatatatcttgtatgcatatgtaggttgtgag

SEQ ID NO: 244 - RNA, 33 nt, synthetic construct
  source 1..33: mol_type = other RNA
gttgcaacttacgcataggtgtaaaatacgagg

SEQ ID NO: 245 - RNA, 32 nt, synthetic construct
  source 1..32: mol_type = other RNA
gatgcaacttagatgcatatgtaagttgtgag

SEQ ID NO: 246 - RNA, 32 nt, synthetic construct
  source 1..32: mol_type = other RNA
gttgcaatgaacgtatgtgcatgaggtgtgag

SEQ ID NO: 247 - RNA, 32 nt, synthetic construct; identical to SEQ ID NO: 243
  source 1..32: mol_type = other RNA

SEQ ID NO: 248 - RNA, 33 nt, synthetic construct; identical to SEQ ID NO: 244
  source 1..33: mol_type = other RNA

SEQ ID NO: 249 - RNA, 32 nt, synthetic construct
  source 1..32: mol_type = other RNA
gttgcaattcgtatgcgcaggtaagtttcgag

SEQ ID NO: 250 - RNA, 17 nt, synthetic construct
  source 1..17: mol_type = other RNA
tgtgcatgaggtgtgag

SEQ ID NO: 251 - RNA, 32 nt, synthetic construct
  source 1..32: mol_type = other RNA
gttgcaatctgcgtacaggcgtaagatgtgag

SEQ ID NO: 252 - RNA, 32 nt, synthetic construct; identical to SEQ ID NO: 246
  source 1..32: mol_type = other RNA

SEQ ID NO: 253 - RNA, 17 nt, synthetic construct
  source 1..17: mol_type = other RNA
tgtgcatgaggtgtgag

SEQ ID NO: 254 - RNA, 17 nt, synthetic construct
  source 1..17: mol_type = other RNA
caggcgtaagatgtgag

SEQ ID NO: 255 - RNA, 32 nt, synthetic construct
  source 1..32: mol_type = other RNA
gatcatatctgcttgtatgggtatgctgcgag

SEQ ID NO: 256 - RNA, 32 nt, synthetic construct; identical to SEQ ID NO: 243
  source 1..32: mol_type = other RNA

SEQ ID NO: 257 - RNA, 32 nt, synthetic construct; identical to SEQ ID NO: 246
  source 1..32: mol_type = other RNA

SEQ ID NO: 258 - RNA, 33 nt, synthetic construct; identical to SEQ ID NO: 244
  source 1..33: mol_type = other RNA

SEQ ID NO: 259 - RNA, 32 nt, synthetic construct; identical to SEQ ID NO: 249
  source 1..32: mol_type = other RNA

SEQ ID NO: 260 - RNA, 17 nt, synthetic construct
  source 1..17: mol_type = other RNA
tgtgcatgaggtgtgag

SEQ ID NO: 261 - RNA, 17 nt, synthetic construct
  source 1..17: mol_type = other RNA
tatgggtatgctgcgag

SEQ ID NO: 262 - RNA, 32 nt, synthetic construct
  source 1..32: mol_type = other RNA
gttgcaacttacgcataggtgtaaaatacgag

SEQ ID NO: 263 - RNA, 32 nt, synthetic construct; identical to SEQ ID NO: 243
  source 1..32: mol_type = other RNA

SEQ ID NO: 264 - RNA, 32 nt, synthetic construct
  source 1..32: mol_type = other RNA
gattatatctgcttgtatgggtatactgcgag

SEQ ID NO: 265 - RNA, 33 nt, synthetic construct; identical to SEQ ID NO: 244
  source 1..33: mol_type = other RNA

SEQ ID NO: 266 - RNA, 32 nt, synthetic construct; identical to SEQ ID NO: 262
  source 1..32: mol_type = other RNA

SEQ ID NO: 267 - RNA, 32 nt, synthetic construct; identical to SEQ ID NO: 246
  source 1..32: mol_type = other RNA

SEQ ID NO: 268 - RNA, 32 nt, synthetic construct; identical to SEQ ID NO: 243
  source 1..32: mol_type = other RNA

SEQ ID NO: 269 - RNA, 33 nt, synthetic construct; identical to SEQ ID NO: 244
  source 1..33: mol_type = other RNA

SEQ ID NO: 270 - RNA, 38 nt, synthetic construct
  source 1..38: mol_type = other RNA
tcagctcacaacctacatatgcatacaagatatatcgt

SEQ ID NO: 271 - RNA, 32 nt, synthetic construct; identical to SEQ ID NO: 246
  source 1..32: mol_type = other RNA

SEQ ID NO: 272 - RNA, 32 nt, synthetic construct; identical to SEQ ID NO: 243
  source 1..32: mol_type = other RNA

SEQ ID NO: 273 - RNA, 33 nt, synthetic construct; identical to SEQ ID NO: 244
  source 1..33: mol_type = other RNA

SEQ ID NO: 274 - RNA, 32 nt, synthetic construct; identical to SEQ ID NO: 249
  source 1..32: mol_type = other RNA

SEQ ID NO: 275 - RNA, 17 nt, synthetic construct
  source 1..17: mol_type = other RNA
tgtgcatgaggtgtgag

SEQ ID NO: 276 - RNA, 32 nt, synthetic construct; identical to SEQ ID NO: 255
  source 1..32: mol_type = other RNA

SEQ ID NO: 277 - RNA, 32 nt, synthetic construct; identical to SEQ ID NO: 243
  source 1..32: mol_type = other RNA

SEQ ID NO: 278 - RNA, 32 nt, synthetic construct; identical to SEQ ID NO: 246
  source 1..32: mol_type = other RNA

SEQ ID NO: 279 - RNA, 33 nt, synthetic construct; identical to SEQ ID NO: 244
  source 1..33: mol_type = other RNA

SEQ ID NO: 280 - RNA, 32 nt, synthetic construct; identical to SEQ ID NO: 249
  source 1..32: mol_type = other RNA

SEQ ID NO: 281 - RNA, 17 nt, synthetic construct
  source 1..17: mol_type = other RNA
tgtgcatgaggtgtgag

SEQ ID NO: 282 - RNA, 32 nt, synthetic construct; identical to SEQ ID NO: 243
  source 1..32: mol_type = other RNA

SEQ ID NO: 283 - RNA, 17 nt, synthetic construct
  source 1..17: mol_type = other RNA
catatgtaggttgtgag

SEQ ID NO: 284 - RNA, 13 nt, synthetic construct
  source 1..13: mol_type = other RNA
tgtaggttgtgag

SEQ ID NO: 285 - RNA, 32 nt, synthetic construct; identical to SEQ ID NO: 246
  source 1..32: mol_type = other RNA

SEQ ID NO: 286 - RNA, 17 nt, synthetic construct
  source 1..17: mol_type = other RNA
tgtgcatgaggtgtgag

SEQ ID NO: 287 - RNA, 13 nt, synthetic construct
  source 1..13: mol_type = other RNA
catgaggtgtgag

SEQ ID NO: 288 - RNA, 13 nt, synthetic construct
  source 1..13: mol_type = other RNA
aggtacatccaac

SEQ ID NO: 289 - RNA, 13 nt, synthetic construct
  source 1..13: mol_type = other RNA
tggtacatccaac

SEQ ID NO: 290 - RNA, 17 nt, synthetic construct
  source 1..17: mol_type = other RNA
tgcggtgtaattcgagg

SEQ ID NO: 291 - RNA, 36 nt, synthetic construct
  source 1..36: mol_type = other RNA
gatgtgaacgacctttttttgcggtgtgcttcgagg

SEQ ID NO: 292 - RNA, 13 nt, synthetic construct
  source 1..13: mol_type = other RNA
aggtacatccaac

SEQ ID NO: 293 - RNA, 36 nt, synthetic construct
  source 1..36: mol_type = other RNA
ggtggatatcatcttaaaaagtgaggtacatccaac

SEQ ID NO: 294 - RNA, 36 nt, synthetic construct; identical to SEQ ID NO: 293
  source 1..36: mol_type = other RNA

SEQ ID NO: 295 - RNA, 13 nt, synthetic construct
  source 1..13: mol_type = other RNA
tggtacatccaac

SEQ ID NO: 296 - RNA, 13 nt, synthetic construct
  source 1..13: mol_type = other RNA
tggtacatccaac

SEQ ID NO: 297 - RNA, 13 nt, synthetic construct
  source 1..13: mol_type = other RNA
tggtacatccaac

SEQ ID NO: 298 - RNA, 36 nt, synthetic construct
  source 1..36: mol_type = other RNA
ggtgtgaacgacctttttttgcggtgtaattcgagg

SEQ ID NO: 299 - RNA, 17 nt, synthetic construct
  source 1..17: mol_type = other RNA
tgcggtgtaattcgagg

SEQ ID NO: 300 - RNA, 18 nt, synthetic construct
  source 1..18: mol_type = other RNA
ttgcggtgtacttcgagg

SEQ ID NO: 301 - RNA, 33 nt, synthetic construct; identical to SEQ ID NO: 244
  source 1..33: mol_type = other RNA

SEQ ID NO: 302 - RNA, 33 nt, synthetic construct; identical to SEQ ID NO: 244
  source 1..33: mol_type = other RNA

SEQ ID NO: 303 - RNA, 37 nt, synthetic construct; identical to SEQ ID NO: 240
  source 1..37: mol_type = other RNA

SEQ ID NO: 304 - RNA, 17 nt, synthetic construct
  source 1..17: mol_type = other RNA
agaagaaggattgggac

SEQ ID NO: 305 - RNA, 68 nt, synthetic construct
  source 1..68: mol_type = other RNA
gatatatcttgtatgcatatgtaggttgtgagaatgtgaacgaccttcttttgcggtgtacttcgagg

SEQ ID NO: 306 - RNA, 13 nt, synthetic construct
  source 1..13: mol_type = other RNA
aggtacatccaac

SEQ ID NO: 307 - RNA, 14 nt, synthetic construct
  source 1..14: mol_type = other RNA
aaggttgatacagc

SEQ ID NO: 308 - RNA, 36 nt, synthetic construct
  source 1..36: mol_type = other RNA
gctgtaagtcatggaaaaatggtgagtacatccaac

SEQ ID NO: 309 - RNA, 18 nt, synthetic construct
  source 1..18: mol_type = other RNA
atggtgagtacatccaac

SEQ ID NO: 310 - RNA, 13 nt, synthetic construct
  source 1..13: mol_type = other RNA
gagcacatccaac

SEQ ID NO: 311 - RNA, 13 nt, synthetic construct
  source 1..13: mol_type = other RNA
gggtacatccaac

SEQ ID NO: 312 - RNA, 13 nt, synthetic construct
  source 1..13: mol_type = other RNA
tggtacatccaac

SEQ ID NO: 313 - RNA, 36 nt, synthetic construct
  source 1..36: mol_type = other RNA
gttgcgtttgcccgtgatttcgggtgtgtatacagg

SEQ ID NO: 314 - RNA, 13 nt, synthetic construct
  source 1..13: mol_type = other RNA
tggtacatccaac

SEQ ID NO: 315 - RNA, 13 nt, synthetic construct
  source 1..13: mol_type = other RNA
aggtatatccaac

SEQ ID NO: 316 - RNA, 36 nt, synthetic construct
  source 1..36: mol_type = other RNA
ggcgtatgtctacctgaaaaagaaggtatatccaac

SEQ ID NO: 317 - RNA, 13 nt, synthetic construct
  source 1..13: mol_type = other RNA
tggtacatccaac

SEQ ID NO: 318 - RNA, 13 nt, synthetic construct
  source 1..13: mol_type = other RNA
ggctacatacagc

SEQ ID NO: 319 - RNA, 36 nt, synthetic construct
  source 1..36: mol_type = other RNA
ggtgtatgtgcaccatatatgtaggtgacatacagc

SEQ ID NO: 320 - RNA, 13 nt, synthetic construct
  source 1..13: mol_type = other RNA
gggtacatccaac

SEQ ID NO: 321 - RNA, 36 nt, synthetic construct; identical to SEQ ID NO: 220
  source 1..36: mol_type = other RNA

SEQ ID NO: 322 - RNA, 17 nt, synthetic construct
  source 1..17: mol_type = other RNA
aaaacaaggattgaaac

SEQ ID NO: 323 - RNA, 36 nt, synthetic construct
  source 1..36: mol_type = other RNA
gatgtgaacgacctttttttgcggtgtacttcgagg

SEQ ID NO: 324 - RNA, 13 nt, synthetic construct
  source 1..13: mol_type = other RNA
gtgtacttcgagg

SEQ ID NO: 325 - RNA, 36 nt, synthetic construct
  source 1..36: mol_type = other RNA
gatgtaaatcatctataaaagaaaggtacatccaac

SEQ ID NO: 326 - RNA, 12 nt, synthetic construct
  source 1..12: mol_type = other RNA
ggtacatccaac

SEQ ID NO: 327 - RNA, 13 nt, synthetic construct
  source 1..13: mol_type = other RNA
gggtacatccaac

SEQ ID NO: 328 - RNA, 13 nt, synthetic construct
  source 1..13: mol_type = other RNA
tggtacatccaac

SEQ ID NO: 329 - RNA, 37 nt, synthetic construct; identical to SEQ ID NO: 240
  source 1..37: mol_type = other RNA

SEQ ID NO: 330 - RNA, 17 nt, synthetic construct
  source 1..17: mol_type = other RNA
cgtacgtggattgaaac

SEQ ID NO: 331 - RNA, 36 nt, synthetic construct
  source 1..36: mol_type = other RNA
gctgcactgcaccgcccattgatggtgtgctctagg

SEQ ID NO: 332 - RNA, 36 nt, synthetic construct
  source 1..36: mol_type = other RNA
attgtaggcgacctttttttgcgatgtagttcgagg

SEQ ID NO: 333 - RNA, 13 nt, synthetic construct
  source 1..13: mol_type = other RNA
atgtagttcgagg

SEQ ID NO: 334 - RNA, 36 nt, synthetic construct
  source 1..36: mol_type = other RNA
agtgtatgattacctgtagtatgaggtacatccaac

SEQ ID NO: 335 - RNA, 13 nt, synthetic construct
  source 1..13: mol_type = other RNA
aggtacatccaac

SEQ ID NO: 336 - RNA, 32 nt, synthetic construct; identical to SEQ ID NO: 249
  source 1..32: mol_type = other RNA

SEQ ID NO: 337 - RNA, 32 nt, synthetic construct; identical to SEQ ID NO: 249
  source 1..32: mol_type = other RNA

SEQ ID NO: 338 - RNA, 17 nt, synthetic construct
  source 1..17: mol_type = other RNA
aaaacaaggattgaaac

SEQ ID NO: 339 - RNA, 13 nt, synthetic construct
  source 1..13: mol_type = other RNA
tggtacatccaac

SEQ ID NO: 340 - RNA, 13 nt, synthetic construct
  source 1..13: mol_type = other RNA
tggtacatccaac

SEQ ID NO: 341 - RNA, 13 nt, synthetic construct
  source 1..13: mol_type = other RNA
tggtacatccaac

SEQ ID NO: 342 - RNA, 36 nt, synthetic construct
  source 1..36: mol_type = other RNA
gctgcaagagctcctaatttgaggggtgcatacagg

SEQ ID NO: 343 - RNA, 36 nt, synthetic construct
  source 1..36: mol_type = other RNA
gatagttttaacttccatttgaaatgtaaatgcaac

SEQ ID NO: 344 - RNA, 13 nt, synthetic construct
  source 1..13: mol_type = other RNA
atgtaaatgcaac

SEQ ID NO: 345 - RNA, 17 nt, synthetic construct
  source 1..17: mol_type = other RNA
gaatgaaggaatgcaac

SEQ ID NO: 346 - protein, 448 aa, unidentified
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 347 - RNA, 13 nt, synthetic construct
  source 1..13: mol_type = other RNA
aaggatgccaaac

SEQ ID NO: 348 - RNA, 69 nt, synthetic construct
  source 1..69: mol_type = other RNA
acagcttatttggaagctgaaatgtgaggtttataacactcacaagaatcctgaaaaaggatgccaaac

SEQ ID NO: 349 - RNA, 61 nt, synthetic construct
  source 1..61: mol_type = other RNA
tggggcagttggttgcccttagcctgaggcatttattgcactcgggaagtaccatttctca

SEQ ID NO: 350 - RNA, 20 nt, synthetic construct
  source 1..20: mol_type = other RNA
accaaaaaatatacgctata

SEQ ID NO: 351 - RNA, 20 nt, synthetic construct
  source 1..20: mol_type = other RNA
aagatagagataaacctttg

SEQ ID NO: 352 - RNA, 20 nt, synthetic construct
  source 1..20: mol_type = other RNA
aaatgttgtgtgtacatgct

SEQ ID NO: 353 - RNA, 20 nt, synthetic construct
  source 1..20: mol_type = other RNA
agataacgtttggaagttac

SEQ ID NO: 354 - RNA, 20 nt, synthetic construct
  source 1..20: mol_type = other RNA
ctgacaaagatatcactctg

SEQ ID NO: 355 - RNA, 20 nt, synthetic construct
  source 1..20: mol_type = other RNA
acaatgtggatactttgttt

SEQ ID NO: 356 - RNA, 28 nt, synthetic construct
  source 1..28: mol_type = other RNA
gggtcttcgaggacgacgaagaagacct

SEQ ID NO: 357 - RNA, 26 nt, synthetic construct
  source 1..26: mol_type = other RNA
agagatatatcagcgtatattttttg

SEQ ID NO: 358 - RNA, 30 nt, synthetic construct
  source 1..30: mol_type = other RNA
agagatatatcagcgtatattttttggtta

SEQ ID NO: 359 - RNA, 31 nt, synthetic construct
  source 1..31: mol_type = other RNA
attatagagatatatcagcgtatattttttg

SEQ ID NO: 360 - RNA, 35 nt, synthetic construct
  source 1..35: mol_type = other RNA
attatagagatatatcagcgtatattttttggtta

SEQ ID NO: 361 - RNA, 26 nt, synthetic construct
  source 1..26: mol_type = other RNA
taacacaaatcaggtttatctctatc

SEQ ID NO: 362 - RNA, 30 nt, synthetic construct
  source 1..30: mol_type = other RNA
taacacaaatcaggtttatctctatcttta

SEQ ID NO: 363 - RNA, 31 nt, synthetic construct
  source 1..31: mol_type = other RNA
gtcaataacacaaatcaggtttatctctatc

SEQ ID NO: 364 - RNA, 35 nt, synthetic construct
  source 1..35: mol_type = other RNA
gtcaataacacaaatcaggtttatctctatcttta

SEQ ID NO: 365 - RNA, 26 nt, synthetic construct
  source 1..26: mol_type = other RNA
cacctagcatcatgtacacacaacat

SEQ ID NO: 366 - RNA, 30 nt, synthetic construct
  source 1..30: mol_type = other RNA
cacctagcatcatgtacacacaacatttta

SEQ ID NO: 367 - RNA, 31 nt, synthetic construct
  source 1..31: mol_type = other RNA
atacacacctagcatcatgtacacacaacat

SEQ ID NO: 368 - RNA, 35 nt, synthetic construct
  source 1..35: mol_type = other RNA
atacacacctagcatcatgtacacacaacatttta

SEQ ID NO: 369 - RNA, 26 nt, synthetic construct
  source 1..26: mol_type = other RNA
taaatgtaatcacttccaaacgttat

SEQ ID NO: 370 - RNA, 30 nt, synthetic construct
  source 1..30: mol_type = other RNA
taaatgtaatcacttccaaacgttatctca

SEQ ID NO: 371 - RNA, 31 nt, synthetic construct
  source 1..31: mol_type = other RNA
cattttaaatgtaatcacttccaaacgttat

SEQ ID NO: 372 - RNA, 35 nt, synthetic construct
  source 1..35: mol_type = other RNA
cattttaaatgtaatcacttccaaacgttatctca

SEQ ID NO: 373 - RNA, 26 nt, synthetic construct
  source 1..26: mol_type = other RNA
actatcagatcagtgatatctttgtc

SEQ ID NO: 374 - RNA, 30 nt, synthetic construct
  source 1..30: mol_type = other RNA
actatcagatcagtgatatctttgtcagta

SEQ ID NO: 375 - RNA, 31 nt, synthetic construct
  source 1..31: mol_type = other RNA
aggaaactatcagatcagtgatatctttgtc

SEQ ID NO: 376 - RNA, 35 nt, synthetic construct
  source 1..35: mol_type = other RNA
aggaaactatcagatcagtgatatctttgtcagta

SEQ ID NO: 377 - RNA, 26 nt, synthetic construct
  source 1..26: mol_type = other RNA
ttgctaaactcaaaagtatccacatt

SEQ ID NO: 378 - RNA, 30 nt, synthetic construct
  source 1..30: mol_type = other RNA
ttgctaaactcaaaagtatccacattgtta

SEQ ID NO: 379 - RNA, 31 nt, synthetic construct
  source 1..31: mol_type = other RNA
atgtattgctaaactcaaaagtatccacatt

SEQ ID NO: 380 - RNA, 35 nt, synthetic construct
  source 1..35: mol_type = other RNA
atgtattgctaaactcaaaagtatccacattgtta

SEQ ID NO: 381 - RNA, 28 nt, synthetic construct
  source 1..28: mol_type = other RNA
gggtcttcgaggacgacgaagaagacct

SEQ ID NO: 382 - RNA, 47 nt, synthetic construct
  source 1..47: mol_type = other RNA
tatctttgtcagatgcaccgaaagatatgcgctgtatctctataatc

SEQ ID NO: 383 - RNA, 55 nt, synthetic construct
  source 1..55: mol_type = other RNA
cagagtgatatctttgtcagatgcaccgaaagatatgcgctgtatctctataatc

SEQ ID NO: 384 - RNA, 49 nt, synthetic construct
  source 1..49: mol_type = other RNA
tatctttgtcagatgcaccgaaagatatgcgctgtatctctataatctg

SEQ ID NO: 385 - RNA, 57 nt, synthetic construct
  source 1..57: mol_type = other RNA
cagagtgatatctttgtcagatgcaccgaaagatatgcgctgtatctctataatctg

SEQ ID NO: 386 - RNA, 53 nt, synthetic construct
  source 1..53: mol_type = other RNA
tatctttgtcagatgcaccgaaagatatgcgctgtatctctataatctgtttt

SEQ ID NO: 387 - RNA, 61 nt, synthetic construct
  source 1..61: mol_type = other RNA
cagagtgatatctttgtcagatgcaccgaaagatatgcgctgtatctctataatctgtttt

SEQ ID NO: 388 - RNA, 47 nt, synthetic construct
  source 1..47: mol_type = other RNA
tttgtttcaatatgcaaggtagggatagaccttggtgttattgactg

SEQ ID NO: 389 - RNA, 55 nt, synthetic construct
  source 1..55: mol_type = other RNA
agagaaaatttgtttcaatatgcaaggtagggatagaccttggtgttattgactg

SEQ ID NO: 390 - RNA, 49 nt, synthetic construct
  source 1..49: mol_type = other RNA
tttgtttcaatatgcaaggtagggatagaccttggtgttattgactgtg

SEQ ID NO: 391 - RNA, 57 nt, synthetic construct
  source 1..57: mol_type = other RNA
agagaaaatttgtttcaatatgcaaggtagggatagaccttggtgttattgactgtg

SEQ ID NO: 392 - RNA, 53 nt, synthetic construct
  source 1..53: mol_type = other RNA
tttgtttcaatatgcaaggtagggatagaccttggtgttattgactgtgcaaa

SEQ ID NO: 393 - RNA, 61 nt, synthetic construct
  source 1..61: mol_type = other RNA
agagaaaatttgtttcaatatgcaaggtagggatagaccttggtgttattgactgtgcaaa

SEQ ID NO: 394 - RNA, 47 nt, synthetic construct
  source 1..47: mol_type = other RNA
attgagtattttatgcaagtgtggtgggtagatggtaggtgtgtata

SEQ ID NO: 395 - RNA, 55 nt, synthetic construct
  source 1..55: mol_type = other RNA
gcttttatattgagtattttatgcaagtgtggtgggtagatggtaggtgtgtata

SEQ ID NO: 396 - RNA, 49 nt, synthetic construct
  source 1..49: mol_type = other RNA
attgagtattttatgcaagtgtggtgggtagatggtaggtgtgtatatt

SEQ ID NO: 397 - RNA, 57 nt, synthetic construct
  source 1..57: mol_type = other RNA
gcttttatattgagtattttatgcaagtgtggtgggtagatggtaggtgtgtatatt

SEQ ID NO: 398 - RNA, 53 nt, synthetic construct
  source 1..53: mol_type = other RNA
attgagtattttatgcaagtgtggtgggtagatggtaggtgtgtatattaatt

SEQ ID NO: 399 - RNA, 61 nt, synthetic construct
  source 1..61: mol_type = other RNA
gcttttatattgagtattttatgcaagtgtggtgggtagatggtaggtgtgtatattaatt

SEQ ID NO: 400 - RNA, 47 nt, synthetic construct
  source 1..47: mol_type = other RNA
aagcaacataaaatgcagataacgtttggaagttacatttaaaatgt

SEQ ID NO: 401 - RNA, 54 nt, synthetic construct
  source 1..54: mol_type = other RNA
ttttaaataagcaacataaaatgcagataacgtttggaagttacattaaaatgt

SEQ ID NO: 402 - RNA, 49 nt, synthetic construct
  source 1..49: mol_type = other RNA
aagcaacataaaatgcagatgacgtgtgggagttgcatttaaaatgtct

SEQ ID NO: 403 - RNA, 57 nt, synthetic construct
  source 1..57: mol_type = other RNA
ttttaaataagcaacataaaatgcagatgacgtgtgggagttgcatttaaaatgtct

SEQ ID NO: 404 - RNA, 53 nt, synthetic construct
  source 1..53: mol_type = other RNA
aagcaacataaaatgcagatgacgtgtgggagttgcatttaaaatgtctcctc

SEQ ID NO: 405 - RNA, 61 nt, synthetic construct
  source 1..61: mol_type = other RNA
ttttaaataagcaacataaaatgcagatgacgtgtgggagttgcatttaaaatgtctcctc

SEQ ID NO: 406 - RNA, 47 nt, synthetic construct
  source 1..47: mol_type = other RNA
atattttttggtatgcctggcaaggatgtcactgtgatagtttcctg

SEQ ID NO: 407 - RNA, 55 nt, synthetic construct
  source 1..55: mol_type = other RNA
tatagcgtatattttttggtatgcctggcaaggatgtcactgtgatagtttcctg

SEQ ID NO: 408 - RNA, 49 nt, synthetic construct
  source 1..49: mol_type = other RNA
atattttttggtatgcctggcaaggatgtcactgtgatagtttcctgca

SEQ ID NO: 409 - RNA, 57 nt, synthetic construct
  source 1..57: mol_type = other RNA
tatagcgtatattttttggtatgcctggcaaggatgtcactgtgatagtttcctgca

SEQ ID NO: 410 - RNA, 53 nt, synthetic construct
  source 1..53: mol_type = other RNA
atattttttggtatgcctggcaaggatgtcactgtgatagtttcctgcatttg

SEQ ID NO: 411 - RNA, 61 nt, synthetic construct
  source 1..61: mol_type = other RNA
tatagcgtatattttttggtatgcctggcaaggatgtcactgtgatagtttcctgcatttg

SEQ ID NO: 412 - RNA, 47 nt, synthetic construct
  source 1..47: mol_type = other RNA
tatatatcttttatgcacagtgtgggtactgtgtgtagcaatacatg

SEQ ID NO: 413 - RNA, 55 nt, synthetic construct
  source 1..55: mol_type = other RNA
catgacattatatatcttttatgcacagtgtgggtactgtgtgtagcaatacatg

SEQ ID NO: 414 - RNA, 49 nt, synthetic construct
  source 1..49: mol_type = other RNA
tatatatcttttatgcacagtgtgggtactgtgtgtagcaatacatggt

SEQ ID NO: 415 - RNA, 57 nt, synthetic construct
  source 1..57: mol_type = other RNA
catgacattatatatcttttatgcacagtgtgggtactgtgtgtagcaatacatggt

SEQ ID NO: 416 - RNA, 53 nt, synthetic construct
  source 1..53: mol_type = other RNA
tatatatcttttatgcacagtgtgggtactgtgtgtagcaatacatggtagaa

SEQ ID NO: 417 - RNA, 61 nt, synthetic construct
  source 1..61: mol_type = other RNA
catgacattatatatcttttatgcacagtgtgggtactgtgtgtagcaatacatggtagaa

SEQ ID NO: 418 - RNA, 90 nt, synthetic construct
  source 1..90: mol_type = other RNA
gtgctcgcttcggcagcacatatactagtcgacgggccgcactcgccggtcccaagcccggataaaatgggagggggcgggaaaccgcct

SEQ ID NO: 419 - RNA, 21 nt, synthetic construct
  source 1..21: mol_type = other RNA
aaccatgccgagtgcggccgc

SEQ ID NO: 420 - RNA, 29 nt, synthetic construct
  source 1..29: mol_type = other RNA
aactaagcacatgaggatcacccatgtgc

SEQ ID NO: 421 - RNA, 35 nt, synthetic construct
  source 1..35: mol_type = other RNA
aaattaacagtggccgcggtcggcgtggactgtag

SEQ ID NO: 422 - RNA, 76 nt, synthetic construct
  source 1..76: mol_type = other RNA
aacactgccaatgccggtcccaagcccggataaaagtggagggtacagtccacgctctagagcggacttcggtccg

SEQ ID NO: 423 - RNA, 280 nt, synthetic construct
  source 1..280: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcgggtcttcgaggacgacgaagaagacctaaattaacagtggccgcggtcggcgtggactgtagaacactgccaatgccggtcccaagcccggataaaagtggagggtacagtccacgctctagagcggacttcggtccgc

SEQ ID NO: 424 - RNA, 278 nt, synthetic construct
  source 1..278: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcagagatatatcagcgtatattttttgaaattaacagtggccgcggtcggcgtggactgtagaacactgccaatgccggtcccaagcccggataaaagtggagggtacagtccacgctctagagcggacttcggtccgc

SEQ ID NO: 425 - RNA, 282 nt, synthetic construct
  source 1..282: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcagagatatatcagcgtatattttttggttaaaattaacagtggccgcggtcggcgtggactgtagaacactgccaatgccggtcccaagcccggataaaagtggagggtacagtccacgctctagagcggacttcggtccgc

SEQ ID NO: 426 - RNA, 283 nt, synthetic construct
  source 1..283: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcattatagagatatatcagcgtatattttttgaaattaacagtggccgcggtcggcgtggactgtagaacactgccaatgccggtcccaagcccggataaaagtggagggtacagtccacgctctagagcggacttcggtccgc

SEQ ID NO: 427 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcattatagagatatatcagcgtatattttttggttaaaattaacagtggccgcggtcggcgtggactgtagaacactgccaatgccggtcccaagcccggataaaagtggagggtacagtccacgctctagagcggacttcggtccgc

SEQ ID NO: 428 - RNA, 278 nt, synthetic construct
  source 1..278: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctaacacaaatcaggtttatctctatcaaattaacagtggccgcggtcggcgtggactgtagaacactgccaatgccggtcccaagcccggataaaagtggagggtacagtccacgctctagagcggacttcggtccgc

SEQ ID NO: 429 - RNA, 282 nt, synthetic construct
  source 1..282: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctaacacaaatcaggtttatctctatctttaaaattaacagtggccgcggtcggcgtggactgtagaacactgccaatgccggtcccaagcccggataaaagtggagggtacagtccacgctctagagcggacttcggtccgc

SEQ ID NO: 430 - RNA, 283 nt, synthetic construct
  source 1..283: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcgtcaataacacaaatcaggtttatctctatcaaattaacagtggccgcggtcggcgtggactgtagaacactgccaatgccggtcccaagcccggataaaagtggagggtacagtccacgctctagagcggacttcggtccgc

SEQ ID NO: 431 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcgtcaataacacaaatcaggtttatctctatctttaaaattaacagtggccgcggtcggcgtggactgtagaacactgccaatgccggtcccaagcccggataaaagtggagggtacagtccacgctctagagcggacttcggtccgc

SEQ ID NO: 432 - RNA, 278 nt, synthetic construct
  source 1..278: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgccacctagcatcatgtacacacaacataaattaacagtggccgcggtcggcgtggactgtagaacactgccaatgccggtcccaagcccggataaaagtggagggtacagtccacgctctagagcggacttcggtccgc

SEQ ID NO: 433 - RNA, 282 nt, synthetic construct
  source 1..282: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgccacctagcatcatgtacacacaacattttaaaattaacagtggccgcggtcggcgtggactgtagaacactgccaatgccggtcccaagcccggataaaagtggagggtacagtccacgctctagagcggacttcggtccgc

SEQ ID NO: 434 - RNA, 283 nt, synthetic construct
  source 1..283: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcatacacacctagcatcatgtacacacaacataaattaacagtggccgcggtcggcgtggactgtagaacactgccaatgccggtcccaagcccggataaaagtggagggtacagtccacgctctagagcggacttcggtccgc

SEQ ID NO: 435 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcatacacacctagcatcatgtacacacaacattttaaaattaacagtggccgcggtcggcgtggactgtagaacactgccaatgccggtcccaagcccggataaaagtggagggtacagtccacgctctagagcggacttcggtccgc

SEQ ID NO: 436 - RNA, 278 nt, synthetic construct
  source 1..278: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctaaatgtaatcacttccaaacgttataaattaacagtggccgcggtcggcgtggactgtagaacactgccaatgccggtcccaagcccggataaaagtggagggtacagtccacgctctagagcggacttcggtccgc

SEQ ID NO: 437 - RNA, 282 nt, synthetic construct
  source 1..282: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctaaatgtaatcacttccaaacgttatctcaaaattaacagtggccgcggtcggcgtggactgtagaacactgccaatgccggtcccaagcccggataaaagtggagggtacagtccacgctctagagcggacttcggtccgc

SEQ ID NO: 438 - RNA, 252 nt, synthetic construct
  source 1..252: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcaaattaacagtggccgcggtcggcgtggactgtagaacactgccaatgccggtcccaagcccggataaaagtggagggtacagtccacgctctagagcggacttcggtccgc

SEQ ID NO: 439 - RNA, 283 nt, synthetic construct
  source 1..283: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgccattttaaatgtaatcacttccaaacgttataaattaacagtggccgcggtcggcgtggactgtagaacactgccaatgccggtcccaagcccggataaaagtggagggtacagtccacgctctagagcggacttcggtccgc

SEQ ID NO: 440 - RNA, 166 nt, synthetic construct
  source 1..166: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcactatcagatcagtgatatctttgtc

SEQ ID NO: 441 - RNA, 282 nt, synthetic construct
  source 1..282: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcactatcagatcagtgatatctttgtcagtaaaattaacagtggccgcggtcggcgtggactgtagaacactgccaatgccggtcccaagcccggataaaagtggagggtacagtccacgctctagagcggacttcggtccgc

SEQ ID NO: 442 - RNA, 283 nt, synthetic construct
  source 1..283: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcaggaaactatcagatcagtgatatctttgtcaaattaacagtggccgcggtcggcgtggactgtagaacactgccaatgccggtcccaagcccggataaaagtggagggtacagtccacgctctagagcggacttcggtccgc

SEQ ID NO: 443 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcaggaaactatcagatcagtgatatctttgtcagtaaaattaacagtggccgcggtcggcgtggactgtagaacactgccaatgccggtcccaagcccggataaaagtggagggtacagtccacgctctagagcggacttcggtccgc

SEQ ID NO: 444 - RNA, 278 nt, synthetic construct
  source 1..278: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcttgctaaactcaaaagtatccacattaaattaacagtggccgcggtcggcgtggactgtagaacactgccaatgccggtcccaagcccggataaaagtggagggtacagtccacgctctagagcggacttcggtccgc

SEQ ID NO: 445 - RNA, 282 nt, synthetic construct
  source 1..282: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcttgctaaactcaaaagtatccacattgttaaaattaacagtggccgcggtcggcgtggactgtagaacactgccaatgccggtcccaagcccggataaaagtggagggtacagtccacgctctagagcggacttcggtccgc

SEQ ID NO: 446 - RNA, 283 nt, synthetic construct
  source 1..283: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcatgtattgctaaactcaaaagtatccacattaaattaacagtggccgcggtcggcgtggactgtagaacactgccaatgccggtcccaagcccggataaaagtggagggtacagtccacgctctagagcggacttcggtccgc

SEQ ID NO: 447 - RNA, 287 nt, synthetic construct
  source 1..287: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcatgtattgctaaactcaaaagtatccacattgttaaaattaacagtggccgcggtcggcgtggactgtagaacactgccaatgccggtcccaagcccggataaaagtggagggtacagtccacgctctagagcggacttcggtccgc

SEQ ID NO: 448 - RNA, 280 nt, synthetic construct; identical to SEQ ID NO: 423
  source 1..280: mol_type = other RNA

SEQ ID NO: 449 - RNA, 299 nt, synthetic construct
  source 1..299: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctatctttgtcagatgcaccgaaagatatgcgctgtatctctataatcaaattaacagtggccgcggtcggcgtggactgtagaacactgccaatgccggtcccaagcccggataaaagtggagggtacagtccacgctctagagcggacttcggtccgc

SEQ ID NO: 450 - RNA, 307 nt, synthetic construct
  source 1..307: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgccagagtgatatctttgtcagatgcaccgaaagatatgcgctgtatctctataatcaaattaacagtggccgcggtcggcgtggactgtagaacactgccaatgccggtcccaagcccggataaaagtggagggtacagtccacgctctagagcggacttcggtccgc

SEQ ID NO: 451 - RNA, 301 nt, synthetic construct
  source 1..301: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctatctttgtcagatgcaccgaaagatatgcgctgtatctctataatctgaaattaacagtggccgcggtcggcgtggactgtagaacactgccaatgccggtcccaagcccggataaaagtggagggtacagtccacgctctagagcggacttcggtccgc

SEQ ID NO: 452 - RNA, 309 nt, synthetic construct
  source 1..309: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgccagagtgatatctttgtcagatgcaccgaaagatatgcgctgtatctctataatctgaaattaacagtggccgcggtcggcgtggactgtagaacactgccaatgccggtcccaagcccggataaaagtggagggtacagtccacgctctagagcggacttcggtccgc

SEQ ID NO: 453 - RNA, 305 nt, synthetic construct
  source 1..305: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctatctttgtcagatgcaccgaaagatatgcgctgtatctctataatctgttttaaattaacagtggccgcggtcggcgtggactgtagaacactgccaatgccggtcccaagcccggataaaagtggagggtacagtccacgctctagagcggacttcggtccgc

SEQ ID NO: 454 - RNA, 313 nt, synthetic construct
  source 1..313: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgccagagtgatatctttgtcagatgcaccgaaagatatgcgctgtatctctataatctgttttaaattaacagtggccgcggtcggcgtggactgtagaacactgccaatgccggtcccaagcccggataaaagtggagggtacagtccacgctctagagcggacttcggtccgc

SEQ ID NO: 455 - RNA, 299 nt, synthetic construct
  source 1..299: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctttgtttcaatatgcaaggtagggatagaccttggtgttattgactgaaattaacagtggccgcggtcggcgtggactgtagaacactgccaatgccggtcccaagcccggataaaagtggagggtacagtccacgctctagagcggacttcggtccgc

SEQ ID NO: 456 - RNA, 307 nt, synthetic construct
  source 1..307: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcagagaaaatttgtttcaatatgcaaggtagggatagaccttggtgttattgactgaaattaacagtggccgcggtcggcgtggactgtagaacactgccaatgccggtcccaagcccggataaaagtggagggtacagtccacgctctagagcggacttcggtccgc

SEQ ID NO: 457 - RNA, 301 nt, synthetic construct
  source 1..301: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctttgtttcaatatgcaaggtagggatagaccttggtgttattgactgtgaaattaacagtggccgcggtcggcgtggactgtagaacactgccaatgccggtcccaagcccggataaaagtggagggtacagtccacgctctagagcggacttcggtccgc

SEQ ID NO: 458 - RNA, 309 nt, synthetic construct
  source 1..309: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcagagaaaatttgtttcaatatgcaaggtagggatagaccttggtgttattgactgtgaaattaacagtggccgcggtcggcgtggactgtagaacactgccaatgccggtcccaagcccggataaaagtggagggtacagtccacgctctagagcggacttcggtccgc

SEQ ID NO: 459 - RNA, 305 nt, synthetic construct
  source 1..305: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctttgtttcaatatgcaaggtagggatagaccttggtgttattgactgtgcaaaaaattaacagtggccgcggtcggcgtggactgtagaacactgccaatgccggtcccaagcccggataaaagtggagggtacagtccacgctctagagcggacttcggtccgc

SEQ ID NO: 460 - RNA, 313 nt, synthetic construct
  source 1..313: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcagagaaaatttgtttcaatatgcaaggtagggatagaccttggtgttattgactgtgcaaaaaattaacagtggccgcggtcggcgtggactgtagaacactgccaatgccggtcccaagcccggataaaagtggagggtacagtccacgctctagagcggacttcggtccgc

SEQ ID NO: 461 - RNA, 299 nt, synthetic construct
  source 1..299: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcattgagtattttatgcaagtgtggtgggtagatggtaggtgtgtataaaattaacagtggccgcggtcggcgtggactgtagaacactgccaatgccggtcccaagcccggataaaagtggagggtacagtccacgctctagagcggacttcggtccgc

SEQ ID NO: 462 - RNA, 307 nt, synthetic construct
  source 1..307: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcgcttttatattgagtattttatgcaagtgtggtgggtagatggtaggtgtgtataaaattaacagtggccgcggtcggcgtggactgtagaacactgccaatgccggtcccaagcccggataaaagtggagggtacagtccacgctctagagcggacttcggtccgc

SEQ ID NO: 463 - RNA, 301 nt, synthetic construct
  source 1..301: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcattgagtattttatgcaagtgtggtgggtagatggtaggtgtgtatattaaattaacagtggccgcggtcggcgtggactgtagaacactgccaatgccggtcccaagcccggataaaagtggagggtacagtccacgctctagagcggacttcggtccgc

SEQ ID NO: 464 - RNA, 309 nt, synthetic construct
  source 1..309: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcgcttttatattgagtattttatgcaagtgtggtgggtagatggtaggtgtgtatattaaattaacagtggccgcggtcggcgtggactgtagaacactgccaatgccggtcccaagcccggataaaagtggagggtacagtccacgctctagagcggacttcggtccgc

SEQ ID NO: 465 - RNA, 305 nt, synthetic construct
  source 1..305: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcattgagtattttatgcaagtgtggtgggtagatggtaggtgtgtatattaattaaattaacagtggccgcggtcggcgtggactgtagaacactgccaatgccggtcccaagcccggataaaagtggagggtacagtccacgctctagagcggacttcggtccgc

SEQ ID NO: 466 - RNA, 313 nt, synthetic construct
  source 1..313: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcgcttttatattgagtattttatgcaagtgtggtgggtagatggtaggtgtgtatattaattaaattaacagtggccgcggtcggcgtggactgtagaacactgccaatgccggtcccaagcccggataaaagtggagggtacagtccacgctctagagcggacttcggtccgc

SEQ ID NO: 467 - RNA, 299 nt, synthetic construct
  source 1..299: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcaagcaacataaaatgcagataacgtttggaagttacatttaaaatgtaaattaacagtggccgcggtcggcgtggactgtagaacactgccaatgccggtcccaagcccggataaaagtggagggtacagtccacgctctagagcggacttcggtccgc

SEQ ID NO: 468 - RNA, 306 nt, synthetic construct
  source 1..306: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcttttaaataagcaacataaaatgcagataacgtttggaagttacattaaaatgtaaattaacagtggccgcggtcggcgtggactgtagaacactgccaatgccggtcccaagcccggataaaagtggagggtacagtccacgctctagagcggacttcggtccgc

SEQ ID NO: 469 - RNA, 301 nt, synthetic construct
  source 1..301: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcaagcaacataaaatgcagatgacgtgtgggagttgcatttaaaatgtctaaattaacagtggccgcggtcggcgtggactgtagaacactgccaatgccggtcccaagcccggataaaagtggagggtacagtccacgctctagagcggacttcggtccgc

SEQ ID NO: 470 - RNA, 309 nt, synthetic construct
  source 1..309: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcttttaaataagcaacataaaatgcagatgacgtgtgggagttgcatttaaaatgtctaaattaacagtggccgcggtcggcgtggactgtagaacactgccaatgccggtcccaagcccggataaaagtggagggtacagtccacgctctagagcggacttcggtccgc

SEQ ID NO: 471 - RNA, 305 nt, synthetic construct
  source 1..305: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcaagcaacataaaatgcagatgacgtgtgggagttgcatttaaaatgtctcctcaaattaacagtggccgcggtcggcgtggactgtagaacactgccaatgccggtcccaagcccggataaaagtggagggtacagtccacgctctagagcggacttcggtccgc

SEQ ID NO: 472 - RNA, 313 nt, synthetic construct
  source 1..313: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcttttaaataagcaacataaaatgcagatgacgtgtgggagttgcatttaaaatgtctcctcaaattaacagtggccgcggtcggcgtggactgtagaacactgccaatgccggtcccaagcccggataaaagtggagggtacagtccacgctctagagcggacttcggtccgc

SEQ ID NO: 473 - RNA, 299 nt, synthetic construct
  source 1..299: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcatattttttggtatgcctggcaaggatgtcactgtgatagtttcctgaaattaacagtggccgcggtcggcgtggactgtagaacactgccaatgccggtcccaagcccggataaaagtggagggtacagtccacgctctagagcggacttcggtccgc

SEQ ID NO: 474 - RNA, 307 nt, synthetic construct
  source 1..307: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctatagcgtatattttttggtatgcctggcaaggatgtcactgtgatagtttcctgaaattaacagtggccgcggtcggcgtggactgtagaacactgccaatgccggtcccaagcccggataaaagtggagggtacagtccacgctctagagcggacttcggtccgc

SEQ ID NO: 475 - RNA, 301 nt, synthetic construct
  source 1..301: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcatattttttggtatgcctggcaaggatgtcactgtgatagtttcctgcaaaattaacagtggccgcggtcggcgtggactgtagaacactgccaatgccggtcccaagcccggataaaagtggagggtacagtccacgctctagagcggacttcggtccgc

SEQ ID NO: 476 - RNA, 309 nt, synthetic construct
  source 1..309: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctatagcgtatattttttggtatgcctggcaaggatgtcactgtgatagtttcctgcaaaattaacagtggccgcggtcggcgtggactgtagaacactgccaatgccggtcccaagcccggataaaagtggagggtacagtccacgctctagagcggacttcggtccgc

SEQ ID NO: 477 - RNA, 305 nt, synthetic construct
  source 1..305: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgcatattttttggtatgcctggcaaggatgtcactgtgatagtttcctgcatttgaaattaacagtggccgcggtcggcgtggactgtagaacactgccaatgccggtcccaagcccggataaaagtggagggtacagtccacgctctagagcggacttcggtccgc

SEQ ID NO: 478 - RNA, 313 nt, synthetic construct
  source 1..313: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctatagcgtatattttttggtatgcctggcaaggatgtcactgtgatagtttcctgcatttgaaattaacagtggccgcggtcggcgtggactgtagaacactgccaatgccggtcccaagcccggataaaagtggagggtacagtccacgctctagagcggacttcggtccgc

SEQ ID NO: 479 - RNA, 299 nt, synthetic construct
  source 1..299: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctatatatcttttatgcacagtgtgggtactgtgtgtagcaatacatgaaattaacagtggccgcggtcggcgtggactgtagaacactgccaatgccggtcccaagcccggataaaagtggagggtacagtccacgctctagagcggacttcggtccgc

SEQ ID NO: 480 - RNA, 307 nt, synthetic construct
  source 1..307: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgccatgacattatatatcttttatgcacagtgtgggtactgtgtgtagcaatacatgaaattaacagtggccgcggtcggcgtggactgtagaacactgccaatgccggtcccaagcccggataaaagtggagggtacagtccacgctctagagcggacttcggtccgc

SEQ ID NO: 481 - RNA, 301 nt, synthetic construct
  source 1..301: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctatatatcttttatgcacagtgtgggtactgtgtgtagcaatacatggtaaattaacagtggccgcggtcggcgtggactgtagaacactgccaatgccggtcccaagcccggataaaagtggagggtacagtccacgctctagagcggacttcggtccgc

SEQ ID NO: 482 - RNA, 309 nt, synthetic construct
  source 1..309: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgccatgacattatatatcttttatgcacagtgtgggtactgtgtgtagcaatacatggtaaattaacagtggccgcggtcggcgtggactgtagaacactgccaatgccggtcccaagcccggataaaagtggagggtacagtccacgctctagagcggacttcggtccgc

SEQ ID NO: 483 - RNA, 305 nt, synthetic construct
  source 1..305: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgctatatatcttttatgcacagtgtgggtactgtgtgtagcaatacatggtagaaaaattaacagtggccgcggtcggcgtggactgtagaacactgccaatgccggtcccaagcccggataaaagtggagggtacagtccacgctctagagcggacttcggtccgc

SEQ ID NO: 484 - RNA, 313 nt, synthetic construct
  source 1..313: mol_type = other RNA
gtgctcgcttcggcagcacatatactagtcgacgggccgcactcgccggtcccaagcccggataaaatgggagggggcgggaaaccgcctaaccatgccgagtgcggccgcaactaagcacatgaggatcacccatgtgccatgacattatatatcttttatgcacagtgtgggtactgtgtgtagcaatacatggtagaaaaattaacagtggccgcggtcggcgtggactgtagaacactgccaatgccggtcccaagcccggataaaagtggagggtacagtccacgctctagagcggacttcggtccgc

SEQ ID NO: 485 - protein, 608 aa, Roseburia intestinalis
VNHYLRLTSLPALYAGWERVAANDGGPGVDRVSINKFDVALDRDLERLHRELVSFTYRCMPLRRYEIPKLNGKIRTLAVPTVRDRVVQSSALIVLQPVIERELERCSFAYRPGLSRLTAIEQIRQLREQGYRWVVDADIESFFDNVEFGLLLRRFRELVPENHTVALIEQWLKAPVLFHGRLIKRTKGLPQGSPISPVLANLFLDKFDEALLAHNHKLIRYADDFIILCKTKPRAEEALRLTEELLAGLHLRLNAEKTAVTSFDQGFKYLGAIFVKSIIIPQSGTKPQRRKKQMNAQSPDPPLRQNEKRRKSAAAKKKEEITVLAQALLEALNAKQMTTADLVRTPPTRPPVPNGAPEPPSAPPNVSPFMRTLYIQEQGCWLRLDGERFVVATGGEEGVALYEIPTVKVEQIMIFGTCLITPAAMRYALLHTIPITLLSSRGQYFGRIESTYGADITLERTQFLHSVDTAFVLQTARTIVQAKLRNTKALLQRHQTKHEMISAAIVQLTRLEDQVANATSVDQVRGYEGSGSAVFFDVFDELMPGKGFTFEKRTRRPPTDPVNAMLSFGYTLLFNNIYSMARLHRLNPYVGSLHAALSEHKQALTCSY

SEQ ID NO: 486 - protein, 362 aa, Roseburia intestinalis
MYRCVKLPVAENMPNTDSQLNTVGWDEIDWRKVERYVYKLHKRIYAASRRGDVKQVRKLQKTLMSSWSNQVLSVRKVTQDNRGKRTAGVDGIHILSSEARINLAGSLKITGKSHPTRRVWIPKPGTEEKRPLGIPTIYDRVVQNLVKTALEPEWEAIFEPNSFGFRPGRSCHDAIWQIKNSIQNRPKFILDADIAKCFDRINHLALLQKLGYTGKIRQQIKAWLKSGVIDRGAFTATSEGTMQGGVISPLLANIALHGMEKMLMEFAKTLDMRRNDKPNCQISWQQKVKSLTFVRYADDFVLIHKDLNVVRRCRDLISEWLKDIGLELKPSKTRIAHTKKIRERGSPEAKRYLYRGGNATRA

SEQ ID NO: 487 - protein, 579 aa, Roseburia intestinalis
MMAHRKIKANHGSKTKGMDKQNVIDFKNMVTEEYLSEIKLELLDYKSGLIRRKEIPKPNGGVRPLGISCYKDKIIEEAIKQVIEPIVEAKFHPDSYGFRPGRSAQEAIQMSQTLVRAGYTYACEFDIKKCFDNIKHRLIRKKLWALGIRDTRLLTIISKRLRANIYDPKTKTVSQSTLGTVQGGVLSPLLANVVLNSLDWWIASQWERFDFGIEENDYKNYASFHAAMNYRLNKTRLKSGRIVRYADDFVIFCKTEMEAKAWYFAVKDWMKCNLKLEISEEKSRIVDLTKDSIDFLGFELKANKEGKKCVKSVDRSNRGSYKYTKTSVTTKNLNKIIQTLKTKIKIILKTSDKKAKAVLITLLNITILGYHNYWRIASESSINFYNIYQRTKLSWWKLRRQADSKTHISPAFRKLYKEYVDSAFSIDGNTIYPIWGCRHTTLNPKKRDYNWYEDSPSIPENISSQMKIMCCNPIIHRSVEYNDNRLSKYSMQLGKDYITGKFVMAQDVHCHHKTMVSKGGKDNFDNLVILSKVTHGWVHQVNPEIPYMTKVQMDRLNDLREKCGREPLKNKKKSLKSEN

SEQ ID NO: 488 - protein, 430 aa, Geobacillus stearothermophilus
MRKWYSLIDKVYARANLLEAFDKVRRNKGGKTSGIDGISVQAFKEHLTANLCQLHEELRSGTYRPQAVKRVYIDKEDGGKRPLGIPTVKDRVVQQALLNVLQPIFEPEFHPSSYGYRPGRSAHHAIAKAERFTRYYGLDHVVVMDLSKCFDTLDHELILSSVNEKVSDGKVLRLIDSFLKSGIIDKGKFEPTEKGSPQGGIISPLLTNIYLNKFDQYMKTLGIRIVRYADDILVFASTKSEAGKYRAIATTYLEGELMLTVNREKTHLTTPYKGIPYLGFEIHNYGVSASEKSIRKFKEKVRKLTPRNQGYKLDYYLTELNFLLKGYSMYFRVARSKSLFRNLMSWVRRRLRMMIMKSWKSWKGLHKQLRRMGYKGSLEKISVTRWRNSSCQLLHMALPNTWFDEQHLFNMEKVTTGTLHQYYNFVLNKV

SEQ ID NO: 489 - protein, 625 aa, Geobacillus stearothermophilus
VNRAVLTLADLARPDALFDAWQQAERRKAGPGTDGQTVQVFGQQVEEHLEGLSKEIREGGYQPRPAQRIWMVKDSGGERGITIFTVRDRVALGAMRRTLGRLIGPTLSTASFAYREGLGALRAAHRLIEHRRSGRGWVVRSDIKEFFDTISHEILLEQLRVFLDARALELFGTILRTPIRDGYGISVPERGVAQGSPISPMLANLYLSGFDAALNCDGRGFVRFADDFVIAALSVKGAAEGLEIAQAQLEGLELRLNDDKTRIVSFEQGFDFLGFHFDADTVRVSETSLAEFKAHLEALLVSRFEQFSPGAVKKANDLIRGWRNYFQLGDVRKDYAALEEWIEARFGHKARQLERLLPDARGQRVPQLGGYGRVSRERQPRRSGQANTASARPQKRQRPYPNVKLDANDTPLTVRRNKAVLNVATLPALPETRVSRAILDELEQASLFHRANTACRWSTPDAADAVSNLAAAMSGRIPIRQAIEAYDKALFAQLKPGADPRGARGTLRANLRRIAWAALLEAGIDLGPEQPRKTPTLGNALADAYECLTADLALLAASRNDQLHAPQGAFERSLAFKPAYQRPGVQGQAHATAWMMILRLEAEAMRRMLETNGQTVYRVWRFGLP

SEQ ID NO: 490 - protein, 417 aa, Geobacillus stearothermophilus
MEAVVERENMIAAYKRVVANRGAAGVDGMTVDALKAYLQPHWPRIRKELLEGRYRPQPVLEVEIPKPAGGMRKLGIPTVVDRLIQQAIHQVLEPIFDKDFSESSYGFRRGKSAHQAVKQAREYVKEGRRWVVDIDLEKFFDRVNHDILMARVARKVRDKRILRTIRQYLQAGIMTGGLVTARTEGTPQGSPLSPLLSNIMLDDWDKELERRGHRFCRYADDCNVYVHSKRAGERVMESLRQFLEKRLRLRVNTEKSRVDHPWQLKFLGYSMTWEKNTRLKVASQSVERFKSKLRLIFRQGRGRNVQIVINELNLVLRGWVQYFRLAEVKNIFEEMDGWIRRKLRCTLWKQWKRTYTRAKNLMKRGLEEVRAWQSATNGRGAWWNSAASHMNEAYPKSYFDKAGLVSLLQLLWQIQLT

SEQ ID NO: 491 - protein, 443 aa, Eubacterium rectale
MSEPMEDKPEAVSERIKRAGELLRHWMWTEPTVWTERMLTALAQGVKGGKWFSLIDKIHPERTLRAAYGHVAANKGSAGVDHVTTTMFGDHLDEEVSKLSEQLRNGSYRPQAIRRHYIPKPGSQEKRPLGIPTVRDRVVQTALRMVIEPIFEREFAEDSYGFRPGRGCKDALRRVDELLKDGYIYIIDADLKSYFDTIPHDRLMDLVRQKISDGRVLNLIEAFLKQGILENLQEWIPESGSPQGAVVSPLLSNIYLDPLDHKMAQEGYQMVRYADDFVILCRTPEKAERALAAVQEWTAAAGLTLHPTKTRIVNATIDSFEFLGYRFSKGKRFPRTKSMQKLKDTIRAKTKRTNGQCLPAIIGSLNPTLRGWFEYFKHSYKTTFPELDGWIRGRLRSILRKRQKKRGRGRGSDHQRWPNAYFADLGLYSLKTAHKLACQSSSR

SEQ ID NO: 492 - protein, 430 aa, Eubacterium rectale
MKGRMQKKSIDTCQQKNRTESDGYVEGQTFIWITENNLTDKYQLGNGLLEFILSPNNLNQAYHQVKRNKGAGGIDKMEVESLKDYLVRHKDELIKSILEGKYRPSPVRRVEIPKETGKTRQLGIPTVVDRVIQQSISQVLSQIYEKQFSDTSYGFRPRRGAHQALRTCQKNITEGYVWSVDMDLEKFFDTVNHSKLIEVLSRTIKDGRVISLIHKYLNAGVIVRNNFEETEVGVPQGGPLSPLLSNIMLNELDREIENRGHRFVRYADDMVILCKSKRSAVRTLENLLPFIEGKLFLKVNKEKTSVAQISKIKFLGYSFYRLNGECQLRVHPKSVLKMREKMKSLTSRSNGWGNERRKESLRQYIVGWVNYFKLANMKKLLLRTDEWYRRRLRMVIWKQWKRISTKLINLIKLGINKYKAWELVNTRKGY

SEQ ID NO: 493 - protein, 428 aa, Eubacterium rectale
MSLATPERIRSLQRKLYCKAKAEPAFRFYTLYDKICRADILAHAYEVARSNAGAPGVDGRTFEHIEALGLQEWLAGLREDLVARTYRPMPVRRVMIPKPGGGERPLGIPTIRDRVIQTAAKLVLEPIFEADFEDSAYGYRPRRGASDAIKEVHRLLCRGHTDVADADLSKYFDTIPHAQLLRCVARRIVDGEVLRLIKLWLKTPIEGRDADGKRRLTGGKRSRCGTPQGGVVSPLLANLYMNRFLKHWRFTGCGEVFRAQIIAYADDFVILSCGHAAEAMMWTKAVMTKLGLNLNEAKTSIRDARKERFEFLGYSFGPHYWWKNGQRYLGASPSKKSVQRIKAKISELLVPGNKGSWPDVRDQLNRLLSGWSAYFNHGTRVPAYRAVDAHVYDRVQNFLCRRHNVRRRGTSRFSFGDVFGELAVLHLM

SEQ ID NO: 494 - protein, 400 aa, Eubacterium rectale
MDEDFLTEAFYQLRKDAAAGIDEMTATEYEVNLGGRIADLHRRLVAQEYRAQPARRVWIPKGDGGQRPLAILVLEDKIVQRAVAMLLEAIYEPHFCGFSYGFRREHNAHQALTYLRQQCLELGINWIIDADIQKFFDTISWEQLRAILQRRMNDGAVLRLIGMWLHVGVLEAGQVINQELGTPQGAPISPILANIFLHIVLDEWFQSQVRPRMRGNCFLVRYADDFVMGYSLKGDAERVYEVLPKRFERYGLRIHPEKSRMVQFSRPYWQRGKGPGSFAFLGFTHYWAKTRSGSWTIKRKTQGKRLSRFLSGIADWCKENLHEAIGEQHRILSAKLRGHYQYYGVRGNFKLLEVAYEHTRGMWKKWLGRRNSMNRMSWEKFVEQVESVFTLPLPRIVHAF

SEQ ID NO: 495 - protein, 424 aa, Geobacillus stearothermophilus
MPKTFYSLYDRLLHRRALARAFAKVRRAKGAKTPGQDGQTAADFAADEEAELDRLVHELRTKTYRPQPVRRVTIPKPGGGERNLGIPAVRDRVVQQSLLDILQPIFDPDFHPSSYGYRPGRSSQQAVAKATRFVRQYGLAHAVDMDLSKCFDRLDHDLIVASFRRRVRDGSILALMRMFLESGVMIGDDWEATEVGSPQGGVISPLISNVYLDAFDQEMKRRGYRIVRYADDILILCRSKSAALHAREVATAILEGPLRLTVNKEKTHLVHASQGVKFLGVEIGLTWTRIQDKKVAAFKAQVKRLTRRNSPVNLGQVIADLNPVLRGFANYFRMANCKGLLSELMSWIRRRLRAKQLALWKKPGRLHRRLRQLGYQGDFPKIRMTRWRNSRSLQASWSLPNGWFRELGLYELTAVETGVLPQAT

SEQ ID NO: 496 - protein, 427 aa, Eubacterium rectale
MSELLEKILSKDNMNTAYKRVCANKGAGGVDDVTVEELGAYVKENWESIREQIRRREYTPQPVRRVEIPKPDGGERKLGIPTVMDRVIQQGIVQIISPMCEPLFSKWSYGFRPNRSCEMAIRQLLEYLNEGYEWIVDIDLEKFFDNVPQDRLMSLVHDIIKDGDTESLIRKYLKAGAMTPQGYKETKLGTPQGGNLSPLLSNIMLNELDKELEARGLRFTRYADDCVIAVKSESSAKRVMRTVTDWIQRKLGLKVNMTKSQITRPQKLKYLGFGFHKDSKAKEWKCRPHQESVKKFKVRLKELTCRKKPGTVTSKIAKINQVTRGWINYYALGSMKTAMTEIDAHLRTRLRVIIWKQWKVPKKRQWGLQKLGVSKDLARLTAYCGDSYYWVATKTCVVRAISKEVFAKSGLVSCLDYYNERHALKLC

SEQ ID NO: 497 - protein, 419 aa, Geobacillus stearothermophilus
MEQVVERSNMLAALKRVERNGGAPGIDGVPTERLRDQIRAEWRRIREELLAGTYRPEPVRRVEIPKPGGGERLLGIPTVMDRLIQQALLQVLTPIFDPQFSDSSYGFRPGRRAHDAVKRARQYVEEGYEWAVDLDIEKFFDRVNHDILMARVARRVTDKRVLTLIRRYLQAGVMVNGVVMETAEGTPQGGPLSPLLANILLDDLDKELEKRGHKFVRYADDCNIYVKSKRAGERVMASVRNFLQERLKLKINEQKSAVDRPWKLKFLGFSMYKPKGGVILIRLASQTIDRVKAKIRGITARNKPISMDERIERLNTYLGGWIGYFALADTPSVFKDIEGWMRRRLRMCLWKQWKRVRTRYRELRALGLPEWVVHHFANARKGPWRMAHGPMNRALGNAYWQSHGLMSLTERYHRLRQAW

SEQ ID NO: 498 - protein, 427 aa, Eubacterium rectale
MSELLERILSNDNMNAAYKRVCANKGAGGVDGVTVEELGDYIKENWSSIREQIRRRQYKPQPVRRVEIPKPNGGVRNLGIPTVMDRVIQQGIVQVLSPMCEPLFSERSYGFRPNRSCEMAIRQLLVYLNEGCEWIVDIDLEKFFDNVPQDRLMSLVHDIINDGDTESLIRKYLKAGVMTPQGYEETKLGTPQGGNLSPLLSNIMLNELDKELEKRGLRFTRYADDCVIAVKSESSAKRVMRTVADWIQRKLGLKVNMTKTHITRPTKLKYLGFGFYRDNKAKEWKCRPHQDSVAKFKRKLKELTCRKTPGTVTGRIAKINQVTRGWINYYAIGSMKTTMIEIDAHLRTRLRVIIWKQWKVPKKRQWGLQKLGIGKDLARLTSYYGDRYQWIVTKTCVARAISKEVLAKAGLVSCLDYYNERHALKLC

SEQ ID NO: 499 - protein, 502 aa, Geobacillus stearothermophilus
VKPREAGMGGPSSSLAQVQTPSCEEGDSLLEKMLERENLLLALRRVEANKGAPGVDGVTVAQLRSYIQTHWADIRQQLLAGTYKPQPVRRVEIPKPGGGVRGLGIPTVIDRFIQQALLQVLNPIFDPEFSDNSFGFRPGRSAHDAVRRAQQYIQQGYRWAVDLDLAKFFDRVNHDKLMARVARKVKDKRVLKLIRAYLNAGIMADGVVVRNEEGTPQGGPLSPLLANIMLDDFDKELKKRGHRFVRYADDCNVYVKSRRAGERVMASLTRYLEGTLKLQVNQEKSAVDRPWKLKFLGFSFLPDKLATIRLAPKTLERFKERVRQITSRSRSMPIAERIRQLNAYIMGWVAYYRLAEMKRHCERFDEWIRRRLRMCIWKQWKRVRTRYRELRALGQPEWVVHMTANSRRGPWFMARMLNQAMDKTYFESTGTAQSSGEIPYTSWCFMNRRMRTRTYGGVRGRGLGAPSYSIGGTSTRCIVAHRIVVTFGQCAPSGFTEVSGGR

SEQ ID NO: 500 - protein, 430 aa, Geobacillus stearothermophilus
MEQLGGDPRYCLMEQVVERANMTKALRRVERNGGAAGVDGMEPEALRSYLKEHWPRIKEELLAGTYRPMPVRRVEIPKPGGGVRLLGIPTVLDRLIQQALLQVLTPIFDPGFSPHSYDFRPGRSAHQAVEQARRYVAQGYRHVVDLDLAQFFDRVNHDLLMARVARKVKDKRVLKLIRAYLQAGVMINGCCVRTEEGTPQGGPLSPLLANIILDDLDKELERRGHKFVRYADDSNVYVRSCRAGQRVFESLKRFLEQRLKLRINEEKSAVDYAWRRGILGFSFTWEKEPRIRLAPQTVKRFKQRIRWLTKRSRSVNMAERLARLNEYLKGWMGYFRLIETPSTLQALDEWIRRRLRMCLLKQWKRPRTRRRNLVALGIPEDWARHISGSRKGCWRLANTPQVNKALGLAYWRSQGLVSLVDRYRELRSAS

SEQ ID NO: 501 - protein, 470 aa, Geobacillus stearothermophilus
VRSREGRRQPKTLNRNCPREEVVKPRGIVGEPSPSPAQSGTSPRGDQGDSLMEKVVARDNMLAALKRVEQNKGAPGVDGIPTENLREQIRAEWPRIREELLAGTYRPQPVRRVEIPKPGGGKRMLGIPTVMDRLIQQALLQVLTPIFDPEFSEASYGFRPGRRAHDAVKKARQYVEEGYEWGVDMDLEKFFDRVNHDVLMARVARKVTDKRVLKVIRRYLQAGIVVNGVVMEREEGTPQGGPLSPLLANILLDDLDKELEKRGHKFVRYADDANIYVRSRRAGERVLASVRKFLQERLKLKLNEQKSTVDRPWKLKFLGFSMYKPKAGEIRIRLARQSIDRVKTKIRALTARNTPIAMEERIRRLNTYLGGWIGYFALAETPSIFEEIDGWIRRRLRMCLWKQWKRVRTRYRELRALGLPEWVVHPFANARKGPWRMAHGPMNRALGNAYWQAQGLMSLTERYRRLRQAW

SEQ ID NO: 502 - protein, 425 aa, Eubacterium rectale
MEKQSMIINNILSNDNLNHAYKQVVKNKGAAGIDGMECEALFSHLRTNGAQLKESIRNQAYNPLPVKRVEISKEDGSKRNLGIPTVTDRLIQQAVAQVLTPIYEKVFHRNSYGFRPGKSAQQAVLKAVEYMNGGYNWVVDIDLEKFFDTVNHNKLISILNKEIKDGKVLSLIRKFLVSGVMVGEHVEETEIGTPQGGNLSPLLANIILNELDWELEKRKLKFVRYADDCIILVRSQKAAQRVMDSISKYIESKLLLKVNRKKSKIGRPIEIKYLGFTFYNQFKAKKYKAKAHEKSVQKVVRKLKQLTSRRWGVSNSVKAQKIAEVLRGWINYFKIGSILTITRKLDTMLRYRFRMCIWKHWKTPQNKYKNLVKLGVEPRNARRAAWSHGYARICRTEAVCYAMSNARLVKFGLLSAEAYFVKVSS

SEQ ID NO: 503 - protein, 427 aa, Roseburia intestinalis
MHEPTLLSQIIHPANLNQAYKQVMKNKGAPGIDDMPITALKAHLALHKETLVHQLQRREYKPQPVKRVEIPKASGGVRLLGIPTVTDRFIQQAIAQVLTPIFDKQFHDNSYGFRPKRYAEMAILQALENMNEGYEWLVDIDLERFFDTVHHDRLMNIVARTVTDGDVISLVRKFLVSGVMVQDEYQETIIGTPQGGNLSPLLSNIMLHELDKELANRDLRFVRYADDCLIFVKSDMAARRVMRSVSKFIEEKLGLIVNVTKSKVRRPEDEETKFLGFGFYYDWNDRLYKARPHKTSLQAIKFKLKCLTRRNWGVPTKSRVERINSLLRGWVNYFKIGKMKKHLIQLDANIRVRLRMCIWKQWKTPKNRRKNLIKLGMNRYEAYKYSYTSKSYARVAYSWILTTTITNQRLTKFGLISATEHYSNIHV

SEQ ID NO: 504 - protein, 425 aa, Geobacillus stearothermophilus
MLDRDNLRLAYKRVVRNGGAPGVDGVTVAALQSYLNTHWDRVKNELLAGTYRPAPVRRVEIPKPGGGVRLLGIPTVMDRFLQQALLQVMNPIFDAHFSWYSYGFRPGKRAHDAVMQAQRYIRDGYRWVVDLDLEKFFDRVNHDMLMARVARKVKDKRVLKLIRAYLNAGVMINGVCQRTEEGTPQGGPLSPLLANILLDDLDKELTRRGLRFVRYADDCNIFVASKRAGERVMESAIRFLEGKLKLKVNRDKSAVDRPWKRKFLGFSFLSDKEATIRLAPKTISRFKERVREITSRSRPIPMEERIRRLNQYTMGWVSYFRLASMKNHCERFDQWIRRRLRMCLWKQWKRVRTRIRELRALGVPDWACFAMANSRRGPWEMSRNINNALPTSYWEAKGLKSMLTRYMVLRQPFGTAWCGPACQVV

SEQ ID NO: 505 - protein, 781 aa, unidentified
  source 1..781: note = AKV murine leukemia virus
VHTHEDRIALSSPPNQADLQIHTGTLSPYTSHWGYSFLDPSIQEAPLLGQSEGQFLPPNLFSYPSGTLSSQEMPDPSPSTPELKVLPNTLKYVFLGPNETLPVIISSKLTMEQECKLTKVLLEHKAAIGWSLSDLSGISPSYCTHRIYTEEEARPSREMQRRLNPNMREVVKNEIIKWLDAGIIYPISDSPWVSPIHVVPKKAGLTIRVNDRGESIPTRTPTSWRVCVDYRKLNAATKKDHFPLPFIDQILERLAGNKFFCFLDGFSGYNQVAIDPRDQEKTTFTCPFGTFTFRRMPFGLCNAPATFQRCMLAIFHDMVGDFLEVFMDDFSVFGPSFDECLANLTRILRRCIDVNLVLSWEKSHFMVHEGIVLGHIISDRGIEVDRAKLDIIADLPVPGSVKQIRSFLGHVGFYRRFIKDFSKISRPLTHLLSTDVPFDMGPDCIQAFTQLRNAVTKAPVLQPPDWSLNFELMCDASDLAVGAILGQRRDRHPFVVYYASKTLDEAQRNYTTTEKELLAVVFGLEKFRSYLLGSHVIVFTDHAAIRHLMHKKDAKPRLIRWILLLQEFDIEIRDKVGAQNVVADHLSRLDPDHIGLTLPINEVFPDEHLLAINVASGPWYADIANYLASGSLPRGWPKNQRERLMAEAKYYFWDNPLLFRIGADQIIRRCIPDSEFPSVLSFCHDQACGGHFSGRKTAAKVLQCGFFWPSLHQDAHTYAKHCTRCQSLGSMARRDMMPLQHILAAEIFDIWGIDFMGPFPPSNGFEFILVAVDYVSKWIEA

SEQ ID NO: 506 - protein, 761 aa, unidentified
  source 1..761: note = AKV murine leukemia virus
ENPIKIDFFEPLEIEPLNLIDVNFVEKMDMDIDLSYEQDSLLKQNLKNLRLDHCNKEEKDAIRKLCFDYRDIFYCEQIPLSFTSEITHKIKLNDESPIYTKSYRFPEIHKKEVKDQIAKMLDQGIIQHSISPWSSPIWVVPKKLDASGRRKWRLVIDYRKLNEKSCNDRYPLPNITDILDKLGRANYFTTLDLASGFHQIQVDPDDIPKTAFSTEGGHYEFKRMPFGLKNAPSTFQRVMDNILRGLHNEICLVYLDDIIIFSTSLDEHIQRLKSVFDRLRKSNFKLQLDKSEFLQKTVQYLGHIITPQGVKPNPDKVSTIKRFPIPRTQKDIKSFLGLLGYYRRFIKDFAKITKPLTKCLKKNAKVTHDQNFIDAFNTCKEILVNDPILQYPDFSKPFILTTDASDVALGAILSQGTLPNDRPVAYASRTLNETESKYSTIEKELLAIVWACKHFRPYLYGRKFTIYTDHRPLTWLFSLKEPNSKLVRWRLKLEEFEYDIIYKKGKLNTNADCLSRVTLNAIDNESMLNNPGDIDQDILDTLQNVTQNLQTLQDFQKPSTSNTNDKINIISDIQIKPPDNSQNDNTNTSTQHSTANETTNDGIKIFDEIINNKTNQILVFPNVIYKLDIKRETYENHKIVTVKIPIINNEQMILQFLKENTDPKHVYCMYFQSDELYNDFCTVYLKHFSEKGPKLIRCLKLVNTVADKEEQILLIKNHHGSKTNHRGINETLEKLKFNYYWKNMKSTVSNFINACDICQRA

SEQ ID NO: 507 - protein, 799 aa, Gibbon ape leukemia virus
MIAMDDEEWGEEDIREFTKLVENSEKAWEPASEKLEVINLGNKQEKKELKIGTLVTTEERNKLVSLLHEYADVFAWTYADMPGLDTDIVVHKIPLIEGSKPVKQKTRRMRPDILLKVKSEIQKQWDAGFLDVVQYPQWVANVVVVPKKDGKIRVCVDYRDLNKASPKDDFPLPHIDILVDNAARNATYSFMDGFSGYNQIRMAEEDKEKTTFVTPWGTFCYKVMPFGLKNAGATYQRAMVTLFHDMMHREVEVYVDDILAKSKDEEDHVQVLRRLFERLRKYQLKLNPAKCSFGVKTGKLLGFIVSGRGIEVDPDKAKAIQEMPAPKTEKKVRSFLGRLNYIARFISQLTVTCEPIFRLLRKKNPGVWDNDCQEAFDKIKRYLQNPPLLVPPTPGRPLILYLTVTETAMGCVLGQHDESGRKEQAIYYLSKKFNDCESRYTTIERLCCALVWSAKRLRQYMLYYTTWLISKLDPLKYICEKPYLSSRIARWQVLLAEYDIVFMTRKAVKGSIIADHLADHAMENYEPLNFDLPDEDVIVIENEGGESDQWTLYFDGAVNVSGNGAGAVVISPENKQYPVSTRLLFECTNNTAEYEACIIGLEAALELKAKKLEVFGDSLLIICQVKGEWQTKDEKLKLYQNYLLRLANEFEEIKFTHISRDKNQFADALATLASMTQVNAKSKIQPIDIEVRSFQAHCCLIEDSLDGKPWYNDIKKFLQHREYPQGISKTDEKTLRRMTMNFYLDGEILYKRSFDGTLLRCLNENEIEQALKEVHEGICAAHANGHTMAKQMQRSGYSG

SEQ ID NO: 508 - protein, 553 aa, Gibbon ape leukemia virus
MDIPVRPVRQRRRKFNEERRLVVKEETQKLLSAGHIREIQYPEWLANVVLVKKANGKWRMCVDFTDLNKASPKDSYPLSSIDALVDSASGCEMLSFLDAFSGYNQIKMHPRDEGKTTFMMETCSYCYKVMPFGLKNAGATYQRLMDKVLAPMLGRNVQAYVDDMVVASHDRRRHTADLEELFVTISKYRLKLNPKKCVFGVEAGKFLGFMLTKRGIEANPDKCAAIIAMRSPTSVKEVQQLTGRMTALSRFVSAGGEKGYHYFQCLKRNSRFAWTDECEAAFVKLKEYLATSPVLCKPVASVPLRLYFAVTERAISSVLVQERNQVQRPIYFVIKALQGPETRYQSLEKAALAVVFSARRLRHYFHSFTIVVMINLPIQKVLQKPDVAGRMVRWAVELSEFDIQYEPRGSIKGQVYADFVAELSPGGEQEVESGSQWLLSVDGSSNQQGSGAGIVLEGPNGVLIEQALRFAFKASNNQDEYEALIAGMLLAKEMGARNLLVKSDSQLITGKVLGEFQAKDPQMAAYLRYVQLLKGAFSALELVHVPREQNARA

SEQ ID NO: 509 - protein, 707 aa, Koala retrovirus
MGSFLLVPEADYNLLGRDLMIELGINLEVRNKEIAVKLYALTAEDEARINPEVWYTPESVGKLDITPFEITIMNPEIPIRVKQYPMSQEGKKGLQSVIQRLLKQGLLEPCMSPYNTPILPIKKPDGSYRLVQDLREVNKRTLTRFPVVANPYTLLSQLSPELKWYSVIDLKDAFWACPLKEECRNYFAFEWEDPETHPKQQFRWTVLPQGFTESSNLFGQALDQVLSTYELNPEVTLVQYVDDLLLAGENQEAVREESIKLLNFLTLKGLKVSKSKLQFTEEEVKYLGHWLSKGTKKLDPERVKGILSLQAPKSKRQVRQLLGLLGYCRQWIENYSKKVKFLYQKLVREGLIKWSQTDKKCFEDLKADLVNAPVLSLPDTKKPFYLFVNTEEGMAYGVLTQKWADRKKLIGYFSKLLDPVSRGWPTCLQAIVATALLVEEAGKVTLGGELKVYTPHNIRGVLQQKAKKWITDARLLKYEGILIHSPKLEIETTSLQNPAQFLYGEPSEELTHDCVRLIESQTKIREDLEEEELPSGEKLFVDGSLRVVNGKRKSVYAIIDGNTSEVKESGPLDTTWSAQACELFAVLKALQLLKGKVGSIFTDSRYAYGVVNTFGKIWEERGLINTQGKNLVHETLIRQTLEALRGPRQVAVVHVKGHQKGASVQIRGNNLADEEAKRAALLIVKETEKPREATGNSKSFTARGIEK

SEQ ID NO: 510 - protein, 753 aa, Koala retrovirus
VGGQSVDFLLDTGATYSVLTEAPGPLSSRSASVMGLSGRAKRYYFSYSLSCNWDSVLFSHEFLIVPESPSPLLGRDILSKVHASVFMNMEPSLSLPLVEQNVNPRVWADGKSVGRAQNAIPVVVKLKDPHVFPHKKQYPLKPEVKEGLKPIIENLKEQGLLIPCNSPCNTPILGIKKSNGKWRLVQDLQIINEAVVPLHPVVPNPYTLLSEIPERAKYFSVIDLKDAFYSVPLAEESQFLFAFEDPTQPASQLTWTVLPQGFRDSPHLFGQSLSRDLQNFNSSEAVVLQYVDDILLCAETEEACSRASEDFLNFLAGCGYKASREKAQLCQQSVRYLGLIISEGTRAIGPERIKPILNHPLPMTLRQLRGFLGITGYCRIWIPGYGELARPLYKLIAETQQAQTDKLVWSPETQKAFKVLQTALLQAPALSLPTGSEFNLFVTERKGVALGVLTQPRGPHQQPIAYLSRELDVVSRGWPHCLRVIGAAALLAPEALKIINGRNLTVLTSHDVSGILNSKVNIWMTDSRLLKYQSLLLEGPVTKLKVCGNLNPATFLPEKENETPDHDCSQFLTLNYAAREDLKDTPLDNPDMEIFTDGSSFVRDGKRKAGYAVVTAEQVLEAKSLPQGTSAQLAELVALTRALELSKGQRMGLIIYVSLLLLTPKILSLPLDPQDNVFLSWAHSYAAFHNRSNCWVCGALPSSSVEGFPWWTSPLQGKDFLQVCEYLRQQSHAMPLLHLMTSTNPKMDWCNTL

SEQ ID NO: 511 - protein, 771 aa, Walleye dermal sarcoma virus
MLSEEALEVLKTVPPCVWSQGKCDVGRMVNAKPVHVRPKTNYRPNIRQYPLKEDAKVGIKPVIEEMLKAGILRECPDASCNTPIFPVKKADGQSWRMIQDLRAVNDAVQSTAPTVPDPHTLMNGLKPNMAWFSVIDLSNAFFSIPVEKDSQGWFGFTHEGTKYTYTRLPQGYVDSPTIFSREIANCLATLQIPDTSQILVYVDDILVASPTQEESTKVTLQVLTHLAKTGNKVSRQKLQFVKEEVIFLGHNLNREGRRIHSERKQTVLNEPKPTTKKQMMQFLGLTNYCRAWIPDYAGMTQPLLDCIYSDNDMKMSGKIEWTQEAEAAFESTKQCLVGSSVLALPDYEKPFVQTVDCKKGFMTSVLAQKHGTKLRPVAYYSKRLDPVAQALPVCVQAVCAAAMAVHCTAEIVLFHPLTLMVPHAVTMLLHDTKMAFLSPARYLALTATLMSQPHIVIKRCNILNPATLIPTAEDGEPHCCKEETDRTCKPRPDLKDIQLLSGETWFVDGSCSKSITGQNQTGFAVVSHSQVIKAGRLPHTYSAQAAELVALTEACKAGVGKDVTMWTDSQYAHSTVHIFAAQWARRGMKTSTGKPVTHAQLLTDLLKAVLLPKSIAICKCAAHTSGKDAVTLGNAHADKVAKLAAMGEYGFHILLQKGESVSQPIPLVILRDMQNSAPDREKKKWLTDGATTDPEGTFRINNKIVLPVSLYKTAAHLSHGPCHVSTGGMVTIINEHFHTYNYITFSKNFCRACVVCCRHNAQGNERPQRGK

SEQ ID NO: 512 - protein, 698 aa, Walleye dermal sarcoma virus
MVMALLAATSAPTVPTVPVELSDISETLWTTISTETGLLKSAEPVYITYHTDIPLPRKMQYPLPKEAIEGIQPVLQQLLTQGIIKEATSPCNTPILPVKKPKPGSYGKPVYRFVHDLRLINAIVIPRAPVIPNPATVLTAIPPEATYFTVIDLSSAFFSNPVHPDSQFLFAFTFDGRQYIWTRLPQGYRESPTIFSQYLKWDLDSIRCAMGSTLIQYVDDLLLCSTAKDACVQDTRILLFALAEKGHKVVLSKLQFAQSEVEYLWHRISYVSTALTQDRIESVLSKPRPTTKKQLHQLLGASGYCRAWIPAYVELVHPLTDLLLDSIPEPLPWTTLHDNALTALKQALTSAPALGRPDYAKPFTLFCHECYGTASGVLTQPFGPGQRMVAYFSVLLDPCAQGLPPCLRAVGAVAMLVETALGLTLGHPLLVQVPHAVAALLNRGSTQHLTAARLTKYELALLANQTITLHRCEVLNPATLLPLPEDGMPHNCLHLMDLISTPRPDLSDVPLQNPDLQLFCDGSAHQDEFGALRVGYAIVILMTTLEAHSLPTQKSAQAAELVALVRACQLAENKTVAIYTDSKYAFSVCHSTGQLWKHHGFLTSCGSQISHAALISALLEAIQLPAAVSVTHCDFVCVKIHRRKHCLEPWWSAPAQVLLTTPTAVKIEGIDRWVHCSHCKKVPAPTSPTTAITTDALT

SEQ ID NO: 513 - protein, 770 aa, unidentified
  source 1..770: note = Friend murine leukemia virus
VFAWDAYDAPGVDPSLICHHLNVNPSSTPKKQPPRRPSKEHASAVKDEVAKLKRAGAIKEVFYPEWLANTVVVKKKTGKWRVCVDFTDLNKACPKDPFPLPRIDRLVDATVGHPRMSFLDAFQGYHQIPLALEDQEKTAFMTPIGNYHYKVMPFGLKNAGSTYQRMMTRMFEPQLGKIIEVYIDDMVVKSRRASEHVKDLGTIFTILREHRLRLNASKCSFGVGSGKFLGYMVTHRGIEVSPDQIKAINSLQAPRNPKEVQKLTGMIAALNRFISRSADRCRPFYLLINKWKEFAWSEDCVQAFQQLKDYLSRPPIMSSPEADEVLFAYIAVAPHAVSLVLIRDDNGVQRPVYYVSKSLHEAEVRYLPLEKAILAIVHATRKLPHYFQAHTVIVLTQLPLRAVLRSADYTGRIAMWSALLGAFDIKYMPRSSIKGQVLADLVAEFAEPSIETITEKRDMDGKSVGAISTGRNTHWKVYVDGAANQRGSGVGIVLISPDGAAIEKSLRLGFSATNNEAEYEALLQGMTMVQRLGGKMIEAFSDSRLVVGQVMGELEARDTRMQEYLGQVKRLQTNFESFNLTHISRSVNTHADSLATLATSSAHNLPRVILVEDLAQASPISRGPARVHQIRKSHSWMDPIKNFLQNDVLPEENWKPRRYEGMLLGFGCQRTTNYIGAPILDRTYSAYTQKNPNLCLRSYTRGFVEVIPEGDRWHTGHLPRGTGGRTCRERPKNTLRSAISAKDSPRISTNPEEFSTPSRVHGRSHNGALI

SEQ ID NO: 514 - protein, 655 aa, unidentified
  source 1..655: note = Friend murine leukemia virus
VLIRDTSITTNPLPARYHDFSDVFEKKNADRLPEHRLYDCPIDLQEGAHPPFGPIYGLAEPELEALREYLKENLAKGFIQPSKSPAGAPILFVKKKDGSLRLCVDYRGLNRVTVRNRYPLPLIPELLDRLRTGRVFSKIDLRGAYNLVRIKPGDEWKTAFRTRYGHFEYKVMPFGLTNAPAVFQHMMNHIFREYLDHFVVIYLDDILVFSPSMEEHTRHVRLILTKLREHGLYAKSEKCEFDRTSVEFLGYVISPAGITMDPRKVATIHNWPIPTRLKEVQSFLGFANFYRRFIDRFSTIVQPLISLTRKDVPFVWTATTQHAFDALKQAFMSAPVLVHPNPAKPFQVETDASDFALGAILSQLDDDGTLHPVAYYSRKFLASEINYPVYDKELAAIISAFAEWRPYLVGAQHRIQVVTDHKNLLYFASSRTLNRRQARWSIFLADYDFEIIFRPGIQHGKADALSRRPDLALCPGDDAYTQQSCSLLKPDQLQLSATFMLHDDSLLQEIAQATKTDTFATEILKRLQDSSPATKRADLHHFTTHDGLLYRNHLLYVPEGPCRTQVLQTCHDDPLAGHFGVAKTLELVSRGFWWPQPWKLVKEFVKTCDVCARSKAVHHRPYGLLHPLPIPNRPWASISMDFITDLPPVNGVDTV

SEQ ID NO: 515 - protein, 631 aa, unidentified
  source 1..631: note = Avian reticuloendotheliosis virus
VSAPDPPVPHADKIIWETQIPVWVDQWPLTQEKLKAASMLVQEQLAADHVEPSTSPWNTPIFVIKKKSGKWRLLQDLRAINRVMRPMGALQPGIPTPVAIPKDYYKMVIDLKDCFFTIPLHPEDRPYFAFSLPRINFQGPMQRFQWKVLPQGMANSPSLCQKYVAQAVDPVRKRWPQIYILHYMDDLLIAAPDTHNLNGCYLDLYKALTTLGLQIAPDKVQTQDPYTYLGLQLQGNQIQTPKIQLRVDHLHTLNDFQKLMGDIQWLRPYLKLPTCVLLPLNDILKGDSHPTSPRKLTPEAQQALNQVTKAITQQFVTQISYNLPLILVILHTPHTPTGIFWQRDPHFKNKGCPLFWVHLPTTSSKVITVYPAQVASVIVKGRLLSRQHFGKDPDKILIPYTKDQTQFLLQTEDEWSIALSGFLGTIDNHLPSDPLLHFAQLHPFIFPKITQKAPIPQALTVFTDGSSNGCAVFVVGNQPTTFETAYQSAQLVELFAIAQVFITFLDKPVNIYTDSAYIAHSVPLLEISPAIKASSNAAFLFHQLQQLIQTRRHPFFLGHIRAHSDLPGPLTQGNAQADAATRSIFPIFAGSIQRATELHTLHHLNSQSLRLFCKITRQQAREIVKACPACT

SEQ ID NO: 516 - protein, 578 aa, unidentified
  source 1..578: note = Avian reticuloendotheliosis virus
VSAPDPPVPHADKIIWETQIPVWVDQWPLTQEKLKAASMLVQEQLAADHVEPSTSPWNTPIFVIKKKSGKWRLLQDLRAINRVMRPMGALQPGIPTPVAIPKDYYKMVIDLKDCFFTIPLHPEDRPYFAFSLPRINFQGPMQRFQWKVLPQGMANSPSLCQKYVAQAVDPVRKRWPQIYILHYMDDLLIATPDTHNLNGCYLDLYKALTTLGLQIAPDKVQTQDPYTYLGLQLQGNQIQTPKIQLRVDHLHTLNDFQKLMGDIQWLRPYLKLPTCVLLPLNDILKGDSHPTSPRKLTPEAQQALNQVTKAITQQFVTQISYNLPLILVILHTPHTPTGIFWQRDPHFKNKGCPLFWVHLPTTSSKVITVYPAQVASVIVKGRLLSRQHFGKDPDKILIPYTKDQTQFLLQTEDEWSIALSGFLGTIDNHLPSDPLLHFAQLHPFIFPKITQKAPIPQALTVFTDGSSNGCAVFVVGNQPTTFETAYQSAQLVELFAIAQVFITFLDKPVNIYTDSAYIAHSVPLLEISPAIKASSNAAFLFHQLQQLIQTRRHPFFLGHIRAHSDLPGPLTQGNAQAD

SEQ ID NO: 517 - protein, 609 aa, unidentified
  source 1..609: note = Xenotropic murine leukemia virus-related virus
VEEAAPIQGNNRDQQAANAMEREIGGKTFKLGRLLSQEKQEGVAEVISRHLDAFAWSASDMPGIDPDFLCHHLSMDVAVRPVRQRRRKYNEERQLVVKEETQKLLSAGHIRVIQYPEWLANVVLVKKANGKWRMCVDFTDLNKACPKDSCPLPSIDALVDSASSCKVLSFLDAFSGYNQIKMHPRDESKTAFMIETCSYCYKVMPFGLKNTGTTYQRLMDKVLAPMLGRNVYAYVDDMVVASQDREHHVADLEELFVTISKYRLKLNPEKCVFGVDAGKFLGFMLTERGKEANPNKCAVIIAMRSPTSVKEVQQLTGRMAALSRFVSAGGQKGHPYFQCLKRNSRFAWTDECEAAFIKLKEYLATPPVLCKSVTGVPLRLYFTVTERAISSVLVQEQDHSQKPISFVSKALQGAETRYQALEKAALAVVFSARRLRHYFHSFTVVVIANLPIQKVLQKPDVAGRMVRWAVELSEFDIQYEPRGSIKGQVYADFVAELSPGGEQEVEVGSQWLLSVDGSSNQQGSGAGIVLEGPNDVLIEQALRFAFKASNNQAEDEALIAGMLLAKEMVAQNLLVKSDSQLITGQVSGEFQAKDPQMAAYLKYVQLLKG

SEQ ID NO: 518 - protein, 767 aa, unidentified
  source 1..767: note = Xenotropic murine leukemia virus-related virus
VYRKQFKIPEAHQTFIEQTLDEWLKLGVVKRSNSLYNSPLFCVPKKQGRGLRIVQDFRELNNHSHIDKYSMKEITECIGDIGRANSSIFSTLDLTSGFWQMKLDEASQPLTAFTIPGQGQFQWITSPMGLLGCPASFQRLMETVLRGIKNVLVYIDDLLIHTATHEEHLIVLEQVFERLHKNHLKINLEKCVFGNPEVSYLGYTLTPNGIKPGRNKLQAIKDAQPPTNIKMVRSFVGLCNFFRTHIKDFAIIAAPLFKVTRKDSNYKSGQLPPDALHAFRVLQLQLTSEPVMAYPRSDRQYALITDAATGTAETPGGLGAILTQIDKSGNHFAISFASRQLKDHEKNYSPFLLEAAAAVWGMDVFNEYLRGKQFILFTDHKPLEKLGHLHTKTLNRLQTALLEHDFVIQYKKGAIMPADYLSRLPTLQVQNVEATVAAFDPFQPDLSKLQVPFQVNTAADVIAAFDPFQPNLKELQFQDTDLQAIYLFLKNGQWLPHLTKRQINSLSALSQKVFFDKNKLAWIRLDDYKYPRTALWLPEFYRKEALCESHNQIFAGHNAAQKTYLKLTSSYFWPNVYSHVLQHTQTCFRCQQRKSSRAKQPPLAPLPIPDLPNVRIHADLFGPMLGVEKKFAYVLCITDAFTKYAMVTKVDNKDAETVARAIFDNWFCKFGIPAQIHTDGGKEFVNKLSAELFELLNVQHSKTSPYHPQCNAQVEVFNKTVKKYLASYVDKTTLNWNDFLPALMLAYNTSYHSTIATTPFELLFGVK

SEQ ID NO: 519 - protein, 697 aa, Woolly monkey sarcoma virus
MKEDHRKIEELVPRKFLKWRKVFGKVESERMPTRKVWDHAIDLKEMFKPQKGRIYPLSKNKREEVQNFVEDQLRKGYIRPSKSPQTLPVFFVGKKDGSKWMVMDYHNLNNQTVKNNYLLPLITELIDNMGSKKVFTKMDLRWGFNNMRIKEGDEWKGVFTMHIGSFEPIVMFFGMTNLPATFQAMINEILRDLINKGKVAAFVDDVLVGTETEKGHDKIVEEILRRLEENDLYVKPEKCIWKVWKIGFLGVVMEPNGIEMEEEKVDRVLSWPELENVKDIRKFLGLANYYRRFIKDFARVARPINMLMRKDVKWQWGVEQQKAFDKLKRVFTTKLVLAAPDLDKEFRVEADVSNYATRGVLSMKCSDEMWRPIAFISKSLSDTERNYEIHDKKMLAVVRCLEAWRYFLEGATTKFEIWTDHKNLEYFMKAQKLNRRQARWALYLSRFNFMLKHVLGSKMGKADSLSRRPDWEVGVEKDNKDQKLVKPEWLEVRKTEMVEIIVDGVDLLEEVRKSKVRDDEVVKVVEEMKKAGVKILRDEEWREADGVMYKKEKVYVPKDNKLRAEIIRLHHDMPVGGHGRQWKMVELVTQNFWWPGITKEVKRYVEGCDACQHNKNRTEQPAGKLMPNSIPEKPWTHISVDFITKLPLVQGYDSILVVVDWLTKMVHFIPTMEKTSAEGLARLFRDNVWKLHGLLES

SEQ ID NO: 520 - protein, 777 aa, Woolly monkey sarcoma virus
MKEDHRKIEELVPRKFLKWRKVFGKVESERMPTRKVWDHAIDLKEMFKPQKGRIYPLSKNKREEVQNFVEDQLRKGYIRPSKSPQTLPVFFVGKKDGSKWMVMDYHNLNNQTVKNNYLLPLITELIDNMGSKKVFTKMDLRWGFNNMRIKEGDEWKGVFTMHIGSFEPIVMFFGMTNLPATFQAMINEILRDLINKGKVAAFVDDVLVGTETEKGHDKIVEEILRRLEENDLYVKPEKCIWKVWKIGFLGVVMEPNGIEMEEEKVDRVLSWPELENVKDIRKFLGLANYYRRFIKDFARVARPINMLMRKDVKWQWGVEQQKAFDKLKRVFTTKLVLAAPDLDKEFRVEADVSNYATRGVLSMKCSDEMWRPIAFISKSLSDTERNYEIHDKKMLAVVRCLEAWRYFLEGATTKFEIWTDHKNLEYFMKAQKLNRRQARWALYLSRFNFMLKHVLGSKMGKADSLSRRPDWEVGVEKDNKDQKLVKPEWLEVRKTEMVEIIVDGVDLLEEVRKSKVRDDEVVKVVEEMKKAGVKILRDEEWREADGVMYKKEKVYVPKDNKLRAEIIRLHHDMPVGGHGRQWKMVELVTQNFWWPGITKEVKRYVEGCDACQHNKNRTEQPAGKLMPNSIPEKPWTHISVDFITKLPLVQGYDSILVVVDWLTKMVHFIPTMEKTSAEGLARLFRDNVWKLHGLLESIISDRGPQFAAGLIRELNGILGIASKLLTVFHPQTNGQTERVNQELEQYLRMFINYRQEQWPEWLGTAEFAYNNKVHSST

SEQ ID NO: 521 - protein, 706 aa, unidentified
  source 1..706: note = Moloney murine leukemia virus
MVKIRGDLSSEMKQQLYNPLKEFKDIFAWSYKDMPGLDFEIVQHRLPLKPECHPIKQRLRRMKPEVSLKIKEEVEKQFNAGFLAVAQYPQWVANVVPVPKKDGSVRMCVDYRDLNRASPKDDFPLPHIDTLVDNTATSSLYSFMDGFSGYNQIKMTPEDMEKITFITLWGTFCYKVMSFGLKNAGATYQRAMMALFHDMMHKEIEVYVDDMIAKSESEEEHLVNLKKLFERLRKYKLRLNPSKCTFGVKSGKLLGFIVSQKGIEVDPDKVRAILEMPHPCTEKQVRGFLGRLNYIARFISQLTATCEPIFKLLRKNQVVKWNENCQNAFDKIKEYLKEPPILHPPVPGRPLILYLTVLDGSMGCVLGQHDGTGKKEHVIYYLSKKFTDCEQRYSSLEKTCCVLAWTAHRLRQYMLSHTTWLVSKMDPIKYIFEKPALTGRIARWQMLLSEFNIVYVIQKAIKGSALAEYLAQQPIDDYQPMQPEFPDEDIMTLFVSEDDGNERKWVLFFDGSSNALGHGIGAVLISPEKQYIPMTARLCFDCTNNIAEYEACAMGIRAAIEYKARRLNVYGDSALVIHQVKGEWETRDQKLIPYKAYIRGLVEYFDEIEFHHISREDNQLADALATLSSMFAISQEEELPMIKMQSHENPVYCNFIEEEVDGKPWYFDIKRYLQNREYPDSASENDKRMLRRLASSFILDGDVLYK

SEQ ID NO: 522 - protein, 706 aa, unidentified
  source 1..706: note = Moloney murine leukemia virus
MVKIRGDLSSEMKQQLYNPLKEFKDIFAWSYKDMPGLDFEIVQHRLPLKPECHPIKQRLRRMKPEVSLKIKEEVEKQFNAGFLAVAQYPQWVANVVPVPKKDGSVRMCVDYRDLNRASPKDDFPLPHIDTLVDNTATSSLYSFMDGFSGYNQIKMTPEDMEKITFITLWGTFCYKVMSFGLKNAGATYQRAMMALFHDMMHKEIEVYVDDMIAKSESEEEHLVNLKKLFERLRKYKLRLNPSKCTFGVKSGKLLGFIVSQKGIEVDPDKVRAILEMPHPCTEKQVRGFLGRLNYIARFISQLTATCEPIFKLLRKNQVVKWNENCQNAFDKIKEYLKEPPILHPPVPGRPLILYLTVLDGSMGCVLGQHDGTGKKEHVIYYLSKKFTDCEQRYSSLEKTCCVLAWTAHRLRQYMLSHTTWLVSKMDPIKYIFEKPALTGRIARWQMLLSEFNIVYVIQKAIKGSALAEYLAQQPIDDYQPMQPEFPDEDIMTLFVSEDDGNERKWILFFDGSSNALGHGIGAVLISPEKQYIPMTARLCFDCTNNIAEYEACAMGIRAAIEYKARRLNVYGDSALVIHQVKGEWETRDQKLIPYKAYIRGLVEYFDEIEFHHISREDNQLADALATLSSMFAISQEEELPMIKMQSHENPVYCNFIEEEVDGKPWYFDIKRYLQNREYPDSASENDKRMLRRLASSFILDGDVLYK

SEQ ID NO: 523 - protein, 688 aa, unidentified
  source 1..688: note = Baboon endogenous virus
MKYPYSNVYSITCYDQIDKCVQQVCDFDCEDGLSITLSYGYDFTKIEEMERHVCVPQNVHESVLALQALQTVPHGNLFVDLVLSHKKLLPSILQAPELELKPLPDNLKYVFIGDNNTLPVIIAKGLTNIQEEKLVKLLCDHKTAIGWTLADIKGISPSMCMHHILLEDNAKPTREMQRRLNPPMMEVVKAEILKLLDAGVIYPITDSKWVAPIHVVPKKTGITLVKNKNDELIPTRISSGWRMCVDYRKLNLSTRKDHFPLPFMDQMLERLAGKSFYCFLDGYSGYNQIVINPEDQEKTTFTCPFGTYAYRRMPFGLCNAPATFQRCMMSIFSDYVERIIEVFMDDFTVYGDSFDKCLENLSLILKRCIETNLVLNYEKCYFMVEQGIVLGHVVSSRGLEVDKAKIDVISSLPYPSCVREVRSFLGHAGFYRRFIKDFSKITAPLCRLLAKEVDFVFDQACKDAHDELKRRVTSAPIIQPPNWDEPFEIMCDASDYAVGAVLGQRIGKNLHVIAYASRMLDGAQCNYHTTEKELFAVVFALEKFRSYLLGTKVIVFTDHAALRYLLKKKESKPRLIRWILLLQEFDLEIKDKKGAENHVADHLSRLRTEDIQIETIRETFPDEQLYMLHSSTRPWYADLVNYLVTKEFPPGLSKPQKDKLRADAKYYFWDDPYLWKFCADQVVRRCVP

SEQ ID NO: 524 - protein, 527 aa, Feline leukemia virus
MPGVPRNLIEHSLNVSKTARPIKQKLRRFARDKKEAIRAEITRLLAAGFIKEVYHPDWLANPVLVRKKNNEWRMCVDYTDLNKHCPKDPFGLPRIDEVVDSTAGCELLSFLDCYSGYHQISLKEEDQIKTSFITPFGAYCYTTMSFGLKNAGATYQRAIQQCLHDEIRDDLVEAYVDDVVVKTRDASTLIDNLDRTFKALNRYKWKLNPKKCIFGVPSGLLLGNVVSRDGIRPNPSKVKAVLDMRPPKNVKDIQKLTGCMAALSRFISRLGEKGLPFFKLLKASEKFSWTEEADTAFNQLKTFLTSPPVLTAPQPNEDLLLYIAATDRVVSTAIVVERDEPGHVFKVQRPVYFISEVLNESKARYPQIQKLIYAILITSRKLKHYFDGHRVLVTTSFPLGDILRNKDANGRIVKWAMELCPFSLEFQSRTTVKSQALVDFIAEWTDLNEPSVPDVSDHWSMFFDGSLNINGAGAGILFVSPNKDKLRYVLRILFPASNNVAEYEACLHGIRLAVELGVKRLYVYGDS

SEQ ID NO: 525 - protein, 572 aa, unidentified
  source 1..572: note = Radiation murine leukemia virus
VPGNEDLDFRKQISPELDEEETEKLVKALGRFVDVFAKGNEPLGMCNMAEHEIPLVPNAKPVYQSLGSGAYKEQLIHRELMSKMKARGAIEVSVGPWGARVLLAQKKDGTWRFCVDYRLLNALTVNSVYPMPSIDGTLARLHGAKIFSIMDLECGYWQIPIKKEDRVKTAFITADGVFQFICMPFGLCTAPSTFQRTMDLVLGGLRWSVCLVYLDDIIVYARNTEEHRQRLVMVLSALRKANLKLKLAKCRFAEKEITALGHRISAEGVRPDPDKVRAVKEFPGLPTSGKRADLVKWVKSFLGLVSYYRRFFPGFAEVAKPLMDLTKDKTLFIWGASHQKSFDELKERLANAAVQAYPDYSLEFEIHPDACGYGVGAVLVQRQMGEERPLAFASRIMTASERNYSITEKECLALVWAVKKFRFFIWGRPIRIVTDHHALCWLQSKKDLAGRLSRWAMQLMEYKYTIAHKDGRLHADADALSRYPLALGLGSDATDKESRDLVVNVVTQCDRSELQEGQRSEWAYVFNNHEQEKETANYTIRNGLLFRIRLWMASRGKLRCDSACRRPCGRGY

SEQ ID NO: 526 - protein, 277 aa, Roseburia intestinalis
MSLMEEVVSRENMQAAYRRVVRNRGAAGPDGMTVEDLADHCRQHWPRIREELLAGRYRPQPVRRVTIPKPGGGERALGIPTVLDRMIQQALLQVLQPVFDPRFSPDSYGFRPGRGAHDAVLRARDHVRGGRRWVVDLDLEKFFDRVNHDVLMSRVARRIEDKGVLRLIRRFLQAGVLEGGVASPRLEGTPQGGPLSPLLSNILLDEWDKELERRGHRFVRYADDCQIYVQSQRAGERVMRSMTRFLEEVLRLRVNRVKSAVARPWERQFLGYGFTQH

SEQ ID NO: 527 - protein, 298 aa, Roseburia intestinalis
MEIETPQRLRKLQQVLHRNAKANSKWRAWSLYADLLREDVLAHAAAKVIANKGAAGIDGTSVDDIRSSESRERFLSDLREELSSKAYKPSPVLCVRIPKKDGKTRALGIPTVKDRVVQPALVLLLEPIFEADMHAESYGYRKGKNAHQAMDSICRALYSGRHEVIDADISGYFDNIDHAMLMKLVKRRVSDGSILKLIKSFLTAPVVENGTGRKNDKGTPQGGVLSPLLANLYLNGLDHQVNGKAGQGAQMVRYADDLVILCHRGKGAELLERLDRYLATKGLSLNRRKTRRVDFTKE

SEQ ID NO: 528 - protein, 345 aa, Geobacillus stearothermophilus
LGIPTLTDRLIQQALHQVLSPIFEAEFSESSYGFRPGRNAHQAVKAAKQYVAEGRRFVVDMDLEKFFDRVNHDVLMAKVAKKVEDGRVLKLIRRYLEAGMMAEGVVSPRTQGTPQGGPLSPLLSNILLTDLDRELERRGLAFCRYADDCNIYVRSQAAGERVLAGISRFLSERLKLRVNEAKSAVARPWERKFLGYSLTWHKTPKLRIAPASLQRLKGKIRKMLKGASGRSVRRTIEELNPVLRGWAAYFKLAETKSALEELDGWLRRKLRCILWRQWKRPYTRARNLMKAGLKEERAFRSAFNQRGPWWNSGASHMNAAFRNVYFDRLGLVSLLDTTRRLQYLS

SEQ ID NO: 529 - protein, 651 aa, unidentified
  source 1..651: note = Baboon endogenous virus
EYKVSIAPIPEYILGIDIMSGLTLQTTVGEFRLRERCISIRAVQAIIRGHAKIEPIFLPQPRCITNTKQYRLSGGEEEFTKIVQELERVGIIRPAHSPYNSPIWPVRKPDGTWRMTVDYRELNKVTPPIHAAVPNIASLMDTLSREIETYHCVLDLANAFFSIPIAKESQDQFAFTWEGRQWTFQVLPQGYVHSPTFCHNLVASDLANWNKPSTVKMFHYIDDLMLTSDSIEALEKTVPSLITYLQEKGWAINPQKVQGPGLSVKFLGVVWSGKTKVLPSAIIDKIQAFPVPTKPKQLQEFLGILGYWRSFIPHLAQLLKPLYRLTKKGQVWDWGRTEQEAFQQAKIAVKQAQALGIFDPTLPAELDVHVTQEGFGWGLWQRQGSVRIPIGFWSQIWHGAEERYSMVEKQLLATYSALQAVEPITQTAEVIVKTTLPIQGWVKDLTHIPKTGVAQSQTVARWVAYLSQRSRLSSSPLKEELQKILGPVTYHSETPEEIVVTCPEESPVQEGKYPIPEDAWYTDGSSRGNPSRWRAVAYHPSTETIWFEEGDGQSSQWAELRAVWMVITQEPGNSALNICTDSWAVYRGLTLWIAQWATQDWTIHARPIWGKDMWVDIWNVVRHRTVRAYHVSGHQPLQSPGNDEADTLARV

SEQ ID NO: 530 - protein, 762 aa, Feline leukemia virus
MIDGLGFANSFQNPSHNSLIFISTYVNKRIMNIMVDTGSQHSFINKNCLREFDKLQSFEISPQNFFMADGMTTFQVKDTIRLNISIGTFDTTAVAFVTTHLCTDMILGMDYLSQYDIEIKTKQNYITFNFGNEQVILSTNLESSSKRHSSSNPELIRSSINQQFHTSALSSINNLEQIISNLANHMTNPTQQNNLKSLLFKFQSTFDTSKYTIAKTEIFHVIETETHIPPASKCYPGNPTLNAELRKIVDKLLDAGLIANSQSPYAAPALLVKKKDNTWRLVIDYKKLNSITLKDNYPLPNMEMALQTLGCGYSYFSKFDLRSGFWQLPIDPKDRFKTAFATPFGLFEWLVLPQGLRNSPPTFQRTMNRVLSACTEFSLVYLDDIVIFSHTFDDHLVHIEKVLSALREHNLTLAPSKCELAKQSIEYLGHVISSKTIAPLPDRIKSIILLPEPKTLTQANKFIGSLSWYRKFIPAFATLAAPIHSITNLSKSNRHKFHWGIDQSKSFAALKQFLTSSPIFLDFPVDGQPIFLATDASLVGLGGVLYQEVQGEKRILYYHSELLTPAQKRYHPLELEALAIFKCVNRMKSFLLGREIFVYTDNCPICHMLDKKVSNKRVEKISILLQEFNIRRIIHVKGEYNCLPDYLSRHPIEQDNEFLNSNYGLEFVRDDSSSIQIAGAVVTRSKAKALPPPPTNSSQTNSQQTTSLISPTSNRDSLNELEPEPESVDMNFDLTQIKQHQLGDVRIQQVIEDLKTNPNSSF

SEQ ID NO: 531 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 532 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 533 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 534 - protein, 1844 aa, synthetic construct
MSELLERILSNDNMNAAYKRVCANKGAGGVDGVTVEELGDYIKENWSSIREQIRRRQYKPQPVRRVEIPKPNGGVRNLGIPTVMDRVIQQGIVQVLSPMCEPLFSERSYGFRPNRSCEMAIRQLLVYLNEGCEWIVDIDLEKFFDNVPQDRLMSLVHDIINDGDTESLIRKYLKAGVMTPQGYEETKLGTPQGGNLSPLLSNIMLNELDKELEKRGLRFTRYADDCVIAVKSESSAKRVMRTVADWIQRKLGLKVNMTKTHITRPTKLKYLGFGFYRDNKAKEWKCRPHQDSVAKFKRKLKELTCRKTPGTVTGRIAKINQVTRGWINYYAIGSMKTTMIEIDAHLRTRLRVIIWKQWKVPKKRQWGLQKLGIGKDLARLTSYYGDRYQWIVTKTCVARAISKEVLAKAGLVSCLDYYNERHALKLCGSSGGSPAGSPTSTEEGTSESATPESGPGTSTEPSEGSAPGSPAGSGGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 535 - protein, 1842 aa, synthetic construct
MLDRDNLRLAYKRVVRNGGAPGVDGVTVAALQSYLNTHWDRVKNELLAGTYRPAPVRRVEIPKPGGGVRLLGIPTVMDRFLQQALLQVMNPIFDAHFSWYSYGFRPGKRAHDAVMQAQRYIRDGYRWVVDLDLEKFFDRVNHDMLMARVARKVKDKRVLKLIRAYLNAGVMINGVCQRTEEGTPQGGPLSPLLANILLDDLDKELTRRGLRFVRYADDCNIFVASKRAGERVMESAIRFLEGKLKLKVNRDKSAVDRPWKRKFLGFSFLSDKEATIRLAPKTISRFKERVREITSRSRPIPMEERIRRLNQYTMGWVSYFRLASMKNHCERFDQWIRRRLRMCLWKQWKRVRTRIRELRALGVPDWACFAMANSRRGPWEMSRNINNALPTSYWEAKGLKSMLTRYMVLRQPFGTAWCGPACQVVGSSGGSPAGSPTSTEEGTSESATPESGPGTSTEPSEGSAPGSPAGSGGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 536 - protein, 2178 aa, synthetic construct
ENPIKIDFFEPLEIEPLNLIDVNFVEKMDMDIDLSYEQDSLLKQNLKNLRLDHCNKEEKDAIRKLCFDYRDIFYCEQIPLSFTSEITHKIKLNDESPIYTKSYRFPEIHKKEVKDQIAKMLDQGIIQHSISPWSSPIWVVPKKLDASGRRKWRLVIDYRKLNEKSCNDRYPLPNITDILDKLGRANYFTTLDLASGFHQIQVDPDDIPKTAFSTEGGHYEFKRMPFGLKNAPSTFQRVMDNILRGLHNEICLVYLDDIIIFSTSLDEHIQRLKSVFDRLRKSNFKLQLDKSEFLQKTVQYLGHIITPQGVKPNPDKVSTIKRFPIPRTQKDIKSFLGLLGYYRRFIKDFAKITKPLTKCLKKNAKVTHDQNFIDAFNTCKEILVNDPILQYPDFSKPFILTTDASDVALGAILSQGTLPNDRPVAYASRTLNETESKYSTIEKELLAIVWACKHFRPYLYGRKFTIYTDHRPLTWLFSLKEPNSKLVRWRLKLEEFEYDIIYKKGKLNTNADCLSRVTLNAIDNESMLNNPGDIDQDILDTLQNVTQNLQTLQDFQKPSTSNTNDKINIISDIQIKPPDNSQNDNTNTSTQHSTANETTNDGIKIFDEIINNKTNQILVFPNVIYKLDIKRETYENHKIVTVKIPIINNEQMILQFLKENTDPKHVYCMYFQSDELYNDFCTVYLKHFSEKGPKLIRCLKLVNTVADKEEQILLIKNHHGSKTNHRGINETLEKLKFNYYWKNMKSTVSNFINACDICQRAGSSGGSPAGSPTSTEEGTSESATPESGPGTSTEPSEGSAPGSPAGSGGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 537 - protein, 2072 aa, synthetic construct
VLIRDTSITTNPLPARYHDFSDVFEKKNADRLPEHRLYDCPIDLQEGAHPPFGPIYGLAEPELEALREYLKENLAKGFIQPSKSPAGAPILFVKKKDGSLRLCVDYRGLNRVTVRNRYPLPLIPELLDRLRTGRVFSKIDLRGAYNLVRIKPGDEWKTAFRTRYGHFEYKVMPFGLTNAPAVFQHMMNHIFREYLDHFVVIYLDDILVFSPSMEEHTRHVRLILTKLREHGLYAKSEKCEFDRTSVEFLGYVISPAGITMDPRKVATIHNWPIPTRLKEVQSFLGFANFYRRFIDRFSTIVQPLISLTRKDVPFVWTATTQHAFDALKQAFMSAPVLVHPNPAKPFQVETDASDFALGAILSQLDDDGTLHPVAYYSRKFLASEINYPVYDKELAAIISAFAEWRPYLVGAQHRIQVVTDHKNLLYFASSRTLNRRQARWSIFLADYDFEIIFRPGIQHGKADALSRRPDLALCPGDDAYTQQSCSLLKPDQLQLSATFMLHDDSLLQEIAQATKTDTFATEILKRLQDSSPATKRADLHHFTTHDGLLYRNHLLYVPEGPCRTQVLQTCHDDPLAGHFGVAKTLELVSRGFWWPQPWKLVKEFVKTCDVCARSKAVHHRPYGLLHPLPIPNRPWASISMDFITDLPPVNGVDTVGSSGGSPAGSPTSTEEGTSESATPESGPGTSTEPSEGSAPGSPAGSGGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 538 - protein, 1844 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSSGGSPAGSPTSTEEGTSESATPESGPGTSTEPSEGSAPGSPAGSGGGSMSELLEKILSKDNMNTAYKRVCANKGAGGVDDVTVEELGAYVKENWESIREQIRRREYTPQPVRRVEIPKPDGGERKLGIPTVMDRVIQQGIVQIISPMCEPLFSKWSYGFRPNRSCEMAIRQLLEYLNEGYEWIVDIDLEKFFDNVPQDRLMSLVHDIIKDGDTESLIRKYLKAGAMTPQGYKETKLGTPQGGNLSPLLSNIMLNELDKELEARGLRFTRYADDCVIAVKSESSAKRVMRTVTDWIQRKLGLKVNMTKSQITRPQKLKYLGFGFHKDSKAKEWKCRPHQESVKKFKVRLKELTCRKKPGTVTSKIAKINQVTRGWINYYALGSMKTAMTEIDAHLRTRLRVIIWKQWKVPKKRQWGLQKLGVSKDLARLTAYCGDSYYWVATKTCVVRAISKEVFAKSGLVSCLDYYNERHALKLC

SEQ ID NO: 539 - protein, 1844 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSSGGSPAGSPTSTEEGTSESATPESGPGTSTEPSEGSAPGSPAGSGGGSMSELLERILSNDNMNAAYKRVCANKGAGGVDGVTVEELGDYIKENWSSIREQIRRRQYKPQPVRRVEIPKPNGGVRNLGIPTVMDRVIQQGIVQVLSPMCEPLFSERSYGFRPNRSCEMAIRQLLVYLNEGCEWIVDIDLEKFFDNVPQDRLMSLVHDIINDGDTESLIRKYLKAGVMTPQGYEETKLGTPQGGNLSPLLSNIMLNELDKELEKRGLRFTRYADDCVIAVKSESSAKRVMRTVADWIQRKLGLKVNMTKTHITRPTKLKYLGFGFYRDNKAKEWKCRPHQDSVAKFKRKLKELTCRKTPGTVTGRIAKINQVTRGWINYYAIGSMKTTMIEIDAHLRTRLRVIIWKQWKVPKKRQWGLQKLGIGKDLARLTSYYGDRYQWIVTKTCVARAISKEVLAKAGLVSCLDYYNERHALKLC

SEQ ID NO: 540 - protein, 1842 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSSGGSPAGSPTSTEEGTSESATPESGPGTSTEPSEGSAPGSPAGSGGGSMLDRDNLRLAYKRVVRNGGAPGVDGVTVAALQSYLNTHWDRVKNELLAGTYRPAPVRRVEIPKPGGGVRLLGIPTVMDRFLQQALLQVMNPIFDAHFSWYSYGFRPGKRAHDAVMQAQRYIRDGYRWVVDLDLEKFFDRVNHDMLMARVARKVKDKRVLKLIRAYLNAGVMINGVCQRTEEGTPQGGPLSPLLANILLDDLDKELTRRGLRFVRYADDCNIFVASKRAGERVMESAIRFLEGKLKLKVNRDKSAVDRPWKRKFLGFSFLSDKEATIRLAPKTISRFKERVREITSRSRPIPMEERIRRLNQYTMGWVSYFRLASMKNHCERFDQWIRRRLRMCLWKQWKRVRTRIRELRALGVPDWACFAMANSRRGPWEMSRNINNALPTSYWEAKGLKSMLTRYMVLRQPFGTAWCGPACQVV

SEQ ID NO: 541 - protein, 2178 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSSGGSPAGSPTSTEEGTSESATPESGPGTSTEPSEGSAPGSPAGSGGGSENPIKIDFFEPLEIEPLNLIDVNFVEKMDMDIDLSYEQDSLLKQNLKNLRLDHCNKEEKDAIRKLCFDYRDIFYCEQIPLSFTSEITHKIKLNDESPIYTKSYRFPEIHKKEVKDQIAKMLDQGIIQHSISPWSSPIWVVPKKLDASGRRKWRLVIDYRKLNEKSCNDRYPLPNITDILDKLGRANYFTTLDLASGFHQIQVDPDDIPKTAFSTEGGHYEFKRMPFGLKNAPSTFQRVMDNILRGLHNEICLVYLDDIIIFSTSLDEHIQRLKSVFDRLRKSNFKLQLDKSEFLQKTVQYLGHIITPQGVKPNPDKVSTIKRFPIPRTQKDIKSFLGLLGYYRRFIKDFAKITKPLTKCLKKNAKVTHDQNFIDAFNTCKEILVNDPILQYPDFSKPFILTTDASDVALGAILSQGTLPNDRPVAYASRTLNETESKYSTIEKELLAIVWACKHFRPYLYGRKFTIYTDHRPLTWLFSLKEPNSKLVRWRLKLEEFEYDIIYKKGKLNTNADCLSRVTLNAIDNESMLNNPGDIDQDILDTLQNVTQNLQTLQDFQKPSTSNTNDKINIISDIQIKPPDNSQNDNTNTSTQHSTANETTNDGIKIFDEIINNKTNQILVFPNVIYKLDIKRETYENHKIVTVKIPIINNEQMILQFLKENTDPKHVYCMYFQSDELYNDFCTVYLKHFSEKGPKLIRCLKLVNTVADKEEQILLIKNHHGSKTNHRGINETLEKLKFNYYWKNMKSTVSNFINACDICQRA

SEQ ID NO: 542 - protein, 2072 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSSGGSPAGSPTSTEEGTSESATPESGPGTSTEPSEGSAPGSPAGSGGGSVLIRDTSITTNPLPARYHDFSDVFEKKNADRLPEHRLYDCPIDLQEGAHPPFGPIYGLAEPELEALREYLKENLAKGFIQPSKSPAGAPILFVKKKDGSLRLCVDYRGLNRVTVRNRYPLPLIPELLDRLRTGRVFSKIDLRGAYNLVRIKPGDEWKTAFRTRYGHFEYKVMPFGLTNAPAVFQHMMNHIFREYLDHFVVIYLDDILVFSPSMEEHTRHVRLILTKLREHGLYAKSEKCEFDRTSVEFLGYVISPAGITMDPRKVATIHNWPIPTRLKEVQSFLGFANFYRRFIDRFSTIVQPLISLTRKDVPFVWTATTQHAFDALKQAFMSAPVLVHPNPAKPFQVETDASDFALGAILSQLDDDGTLHPVAYYSRKFLASEINYPVYDKELAAIISAFAEWRPYLVGAQHRIQVVTDHKNLLYFASSRTLNRRQARWSIFLADYDFEIIFRPGIQHGKADALSRRPDLALCPGDDAYTQQSCSLLKPDQLQLSATFMLHDDSLLQEIAQATKTDTFATEILKRLQDSSPATKRADLHHFTTHDGLLYRNHLLYVPEGPCRTQVLQTCHDDPLAGHFGVAKTLELVSRGFWWPQPWKLVKEFVKTCDVCARSKAVHHRPYGLLHPLPIPNRPWASISMDFITDLPPVNGVDTV

SEQ ID NO: 543 - protein, 50 aa, synthetic construct
GSSGGSPAGSPTSTEEGTSESATPESGPGTSTEPSEGSAPGSPAGSGGGS

SEQ ID NO: 544 - protein, 1367 aa, synthetic construct; identical to SEQ ID NO: 533

SEQ ID NO: 545 - protein, 427 aa, Eubacterium rectale; identical to SEQ ID NO: 496

SEQ ID NO: 546 - protein, 427 aa, Eubacterium rectale; identical to SEQ ID NO: 498

SEQ ID NO: 547 - protein, 427 aa, Clostridium symbiosum
MSELLEKILSKDNMNAAYKRVCANKGAGGVDDVTVEELGAYVKENWESIREQIRRRKYTPQPVRRVEIPKPDGGERKLGIPTVMDRVIQQGIVQIISPMCEPLFSKWSYGFRPNRSCEMAIRQLLEYLNEGYEWIVDIDLEKFFDNVPQDRLMSLVHDIIKDGDTESLIRKYLKAGVMTPQGYKETKLGTPQGGNLSPLLSNIMLNELDKELEARGLRFTRYADDCVIAVKSESSAKRVMRTVTDWIQRKLGLKVNMTKSQITRPQKLKYLGFGFHKDSKAKEWKCRPHQESVKKFKVRLKELTCRKKPGTVTSKIGKINQVTRGWINYYALGSMKTAMTEIDAHLRTRLRVIIWKQWKVPKKRQWGLEKLGVSKELARRTAYCGDSYYWVATKTCVVRAISKEVFAKSGLVSCLDYYNERHALKLC

SEQ ID NO: 548 - protein, 427 aa, unidentified
  source 1..427: note = Lachnospiraceae bacterium
MSELLEKILSKDNMNAAYKRVCANKGAGGIDDVTIEELGDYIKENWESIREQIRHREYKPQPVRRVEIPKPNGGVRELGIPTVMDRVIQQGIVQIISPMCEPLFSEWSYGFRPNRSCEMAVRQLLVYLNEGYEWIVDIDLEKFFDNVPQDRLMSLVHDIIKDGDTESLIRKYLKAGAMTPQGYEETNLGTPQGGNLSPLLSNIILNELDKELEGRGLRFTRYADDCVIAVRSESSAKRVMRTVADWIQRKLGLKVNMTKTRIARPQKLKYLGFGFYKDSKAKEWKCRPHQESVKKLKDRLKELTCRKKPGTVTSKIAEINQVTRGWINYYALGSMKTAMTEIDAHLRTRLRIIIWKQWKVPKKRQWGLQKLGIGKDTARLTAYCGDRYYWVATKTCVVRAISKDVFAKAGLVSCLDYYNERHALKLC

SEQ ID NO: 549 - protein, 427 aa, unidentified
  source 1..427: note = Lachnospiraceae bacterium
MSELLEKILSKENMNAAYKRVCANKGAGGVDGVTVEELGDYIKEHWRSIREQIRQRQYKPQPVRRVEIPKQNGGVRKLGIPTVMDRVIQQGIVQVINPMCEPLFSEWSYGFRPNRSCEMAIRQLLIYLNEGYEWIVDIDLEKFFDNVPQDRLMSLVHVIINDGDTESLIRKYLKAGVMTPQGYEETRLGTPQGGNLSPLLSNIMLNELDKELEARGLRFTRYADDCVIALKSESSAKRVMRTVSDWIQRKLGLKVNMTKTHITKPLKLKYLGFGFYKDSKTKEWKCRAHQDSIVKLKRKLKELTCRKMPGTVREKIEKINQVTRGWINYYALGSMKTAMTAIDAHLRTRLRVIIWKQWKVPKRRQWGLQKLGVGKDLARLTSYCGDRYQWVVTKTCVVRAISKEVLTKAGLISCLEYYNKRHALKLC

SEQ ID NO: 550 - protein, 427 aa, unidentified
  source 1..427: note = Oribacterium asaccharolyticum
MSELLEKILSKENMNTAYKRVCANKGAGGVDGVTVEELGDYIKEHWRSIGEQIRQRQYKPQPVRRVEIPKQNGGVRKLGIPTVMDRVIQQGIVQVINPMCEPLFSEWSYGFRPNRSCEMAIRQLLIYLNEGYEWIVDIDLEKFFDNVPQDRLMSLVHVIINDGDTESLIRKYLKAGVMTPQGYEETRLGTPQGGNLSPLLSNIMLNELDKELEARGLRFTRYADDCVIAVKSETSAKRVMRTVSDWIQRKLGLKVNMTKTHITRPLELKYLGFGFYKDSKTKEWKCRAHQDSIVKIKRRLKELTCRKTPGKVREKIEKINQVTRGWINYYALGSMKTAMTAIDAHLRTRLRVIIWKRWKVPKKRQWGLQKLGIGKDLARLTSYCGDRYQWVVTKTCVVRAISEEVLAKAGLISCLEYYNKRHALKLC

SEQ ID NO: 551 - protein, 427 aa, unidentified
  source 1..427: note = Clostridiales bacterium
MSELLDKILHKDNMNEAYKKVCANKGAGGVDGVSTEELGEYIKTNWPTIKEQILRREYKPQPVRRVEIPKPNGGKRKLGIPTAMDRVIQQAIVQVIGPICDPHFSEFSYGFRPNRSCEMAIRKVLEYLNEDYTWIVDIDLEKFFDNVPQDRLLSYVHILINDGDTESLIRKYLKVGVMTPEGYEHTEMGTPQGGNLSPLLSNIMLNELDKELENRGLHFARYADDCIIAVKSEASAKRVMNSITSWIERKLGLKVNAEKTKIVRPTKVKYLGFGFYKDTKTLEWKCKPHQDSVAKFKRRLKALTQRKNSMRLGIRIKKINQVTRGWINYFALGSMKMAISDIDAHLRTRLRTIIWKQWKVPSKRQWGLQKLGVSKDLARLTSYCGDRYQWVVTKTCVKRAISKEVLGRAGLISCLDYYNERHALKLN

SEQ ID NO: 552 - protein, 427 aa, unidentified
  source 1..427: note = Clostridiales bacterium
MSELLDKILDRENMNEAYKRVCANKGAGGVDGVSTEELGDYIRKNWPEIKEQIKRREYKPQPVRRVEIPKPSGGKRKLGIPTAMDRVIQQAIVQAIGPICDPHFSEFSYGFRPKRSCEMAIRKVLEYLNEDYTWIVDIDLEKFFDNVPQDRLMSYLHILINDGDTESLICKYLKAGVMTPEGYERTEMGTPQGGNLSPLLSNIMLNELDKELEHRGLHFARYADDCIIAVKSEASAKRVMYSITSWIERKLGLKVNAEKTKIVRPTNLKYLGFGFYKDYKADEWKCKPHQEAIAKFKRKLKALTQRKSSMKLGLRIEKINQVTRGWINYFALGSMKMALADIDAHLRTRLRTIIWKQWKVPSKRQWGLQKLGVNKDLARLTSYRGNRYQWVVTKTCVVRAISKEVLGKAGLISCLDYYTERHALKLS

SEQ ID NO: 553 - protein, 427 aa, unidentified
  source 1..427: note = Enterocloster aldenensis
MSELLEKILDKRNMNEAYKKVCANKGAGGVDGMEIDELDGYIRGNWESIKEQIRKRSYTPQPVRRVEIPKSNGSKRKLGIPTVMDRVIQQGIAQVISPMCEPLFSGNSYGFRPNRSCEMAIREMLVFLNEGYEWIVDIDLEKFFDNVPQDRLMSLVHNIINDGDTESLIRKYLKAGVMIQGRYEKTDRGTPQGGNLSPLLSNIMLNELDKELEKRGLRFVRYADDCVIAVRSEASAKRVMYSITDWIERKLGLKVNAEKTHITRPGKLKYLGFGFYKDPKAKEWKSRPHGESVAKFRRTLKKLTNRSQSMAFAERVQKLNRVIRGWINYFALGSMKTAMNDIDAHLRTRLRVIIWKQWKVPSKRQWGLQKLGIGEDLARVTAYCGNRYQWIVTKTCVVRAISKEKLSQAGLVSCYDYYMERHALKLC

SEQ ID NO: 554 - protein, 427 aa, Mogibacterium sp.
MSELLDRILSRDNMNLAYKRVKANKGAGGIDDVEVDELYSYIKENWKSIEGQIRQRTYKPQPVRRVEIPKPNGGKRELGIPTVMDRVIQQAIVQVISPMCEPYFSDRSYGFRPNRSCETAIRKVLEYLNDGYTWIVDIDLEKFFDNVPQDRLMSLVHRIIDDGDTESLIRKYLKAGVMVRGKYERTELGTPQGGNLSPLLSNIMLNELDRELEKRGLNFTRYADDCIIAVRSEASAKRVMHRITEWIERKLGLKVNATKTKITRPSGLKYLGFGFYRDPKADEWKCKPHKDSIAKFKHKLKQLTERRWSINFRVRIAKINQVTRGWINYYALGSMKTAMAEIDAHLRTRLRMIIWKQWKVPAKRQWGLQKLGIGKDLARVTAYCGNRYYWVVTKTCVVRAISKEKLALAGIVSLSDYYAERHMLKLI

SEQ ID NO: 555 - protein, 426 aa, unidentified
  source 1..426: note = Clostridiaceae bacterium
MSEMLERILDDENIKTAYKRVYANKGAGGVDGVTTEELEEYMRVNWRSIKEQIRERKYNPQAVKRVEIPKPNGGIRKLGIPTVIDRVIEQAITQILTPIFDPLFSDSSYGFRPNRRCEQAIIKLLEHFNDGYVWIVDIDLEKFFDNVPQDKLMSYVGRVIHDGDTESLIRKYLKAGVMVNGRYEATNLGTPQGGNLSPLLSNIMLDELDKELENRGLHFTRYADDCVIVLKSKAAATRVMYSITDWIERKLGLKVNATKTKITPPNKLKYLGFGFWKDKQEWKARPHEDSVEKLKRKLKALCKRNWSIDLTTRIKKINEVTRGWINYFRIASMKSKLQNIDEHLRTQLRVIIWKQWKVPKKREWGLAKLGVGKDLARLTAYCGDRYQWVVTKTCVVRAISKEKLTKKGLVSCLDYYAERRHILNLN

SEQ ID NO: 556 - protein, 412 aa, unidentified
  source 1..412: note = Bacteroidales bacterium
MSELLEKILDRDNMNAAYKRVCANKGAGGIDGVTVEELSDYIKENWGSIREQIRQRKYKPQPVRRVEIPKLNGGVRKLGIPTVMDRVIQQGIVQVVSPMCEPMFSERSYGFRPNRSCEMAIRQLLVYLNEGYEWIVDIDLEKFFDNVPQDRLMSLVHDIVKDGDTESLIRKYLKAGVMTRQGYEETNLGTPQGGNLSPLLSNIMLDKLDKELEARGLRFARYADDCVIAVKSEASAKRVMHTVTDWIQRKLGLKVNMTKTHITRPMKLKYLGFGFYKDNKTEEWKCRPHKDSIVRLKSKLKELTCRRMPGKVSEKIKRINQVTRGWINYYALGSMKTVMAEVDAHLRTRLRMNIWKQWKVPKKRQWGLQKLGVDKDSARLTAYYGNHYYKVVKGTCVKRAISKEVLARAGLI

SEQ ID NO: 557 - protein, 424 aa, unidentified
  source 1..424: note = Paludibacteraceae bacterium
MLEKILSKDNMNKAYKAVAANKGSSGVDNVTIEELGSFIKEYWIEIREQIRQRQYKPQPVRRVEIPKPNGGIRKLGIPTVMDRVIQQAIVQVISPICEPYFSDRSYGFRPGRSCEMAIVKLLEYLNDGYEWIVDIDLEKFFDNVPQDKLMSYVHNIIDDGDTESLIRKYLKAGVMTPEGYEETRLGTPQGGNLSPLLSNIMLNELDKELEARGLNFARYADDCVIAAKTDMSARRIMRNITSWIERKLGLKVNATKTKVVRPTKLKYLGFGFYKDIQTKKWMSRPHEESVEKFKRKLKQLTCRSTSMKFKIRINKLNQVIRGWINYFSLGSMKTKMKEIDEHLRTRLRVVLWKQWKKPSKRQWALQKLGIGSDLARQTAYMGDHYQWVVTKTCVVRAISKENLGQFGLVSCLDYYTERHSLKFN

SEQ ID NO: 558 - protein, 439 aa, unidentified
  source 1..439: note = Oscillospiraceae bacterium
MLQLLEEVLDRNNMNLAYEKVYANKGAGGVDGVTLDELKAYIKENWPDVQTQIRNRTYQPKPVKRVEIPKANGGTRNLGIPTVMDRVIQQAMVQVLSPICEPHFSEYSYGFRPKRSCEMAILKLLDYFNDGFLWIVDIDLEKFFDNVPQDRLMSYVHNIINDGDTESLIRKYLKAGVMINGVKHKTEVGTPQGGNLSPLLSNIMLTELDKELEARGLKFTRYADDCIIVVKSEASAKRVMRTITDWIERKLGLKVNMTKTQVTGPTKLKYLGFGFYFDYKSKTWKTRPHEDSVTKFKRKLKQLTKRNWSINLRERIKKINGVIRGWINYFSLCSMKTHMEKIDRHLRTRIRVIVWKQWKVPSKRQWGLQKLGIGRDLAKQTSYMGNHYQWVVTKTCVVRAISKEKLAQAGLVHCLDYYLNQHTLKFNRTAVCRTACTVV

SEQ ID NO: 559 - protein, 449 aa, Eubacterium sp.
MSELLEKILNRENMNKAYKRVKANKGTSGIDEITIEDAYVYIKENWESIRAEITERKYKPQPVKRVEIPKPNGGTRNLGIPTVMDRIIQQAMVQVLSPICETFFSDYSYGFRPNRSCEQAINKLLEYINDGYEWIVDIDLEKFFDNVPQDKLMSYVHIIINDGDTESLIRKYLKAGIMINGKYEKSEKGTPQGGNLSPLLSNIILNELDKELESRGLHFTRYADDCVIAVKSRASANRVMHTITKWIEHKLGLKVNATAAAGLKVNATKTHITRPNKLKYLGFGFYYDTKAEKYCARPHASSIQRFKRKLKQLTIRKNTMALKERIRRLNQVIRGWINYYSICNMKTHMTRIDEHLRTRLRVIIWKQWKVPSKRQWGLQKLGISKDRARQTSYMGDHYQWVVTKTCVVRAISKEKLAQKGLVSCLDYYIERHALKLKRTAVYGTVRTVV

SEQ ID NO: 560 - protein, 439 aa, unidentified
  source 1..439: note = Clostridioides difficile
MSELLEKILNRDNMNQAYKRVKANKGTSGIDEVTVNEAYGYIEENWERIKQEITERRYKPQPVKRVEIPKPNGGTRNLGIPTVMDRIIQQAMVQVLSPMCEEIFSEYSYGFRPGRSCEQAIIKVLEYFNDGYGWIVDIDLEKFFDNVPQDKLMSYVHTIINDGDTESLIRKFLKAGIMINGRYEESEKGTPQGGNLSPLLSNIILSELDKELESRGLKFVRYADDCVIAVKSRASANRVMHTITSWIEGKLGLKVNATKTQITKPNKLKYLGFGFYYHPKEKKYCARPHESSIQQFKRKLKRLTIRKNTMTLEVRIKQLNQVIRGWINYYAIGNMKTHMARIDSHLRTRLRVIIWKQWKVPSKRQWGLQKLGINKDRARQTSYMGDHYQWVVTKTCVVRAISKERLTQKGLVSCLDYYMDRHNLKLERTAVYGTVRTVV

SEQ ID NO: 561 - protein, 453 aa, unidentified
  REGION 1..453: note = group II intron reverse transcriptase
MRNVNEQILHYLGRIHENGKEKAHKAMSELLEKILSKDNMNAAYKRVYANKGAGGVDDVTVGELGGYIKENWESIREQIRRREYKPQPVRRVEIPKPNGGVRELGIPTVMDRVIQQGIVQVISPVCEPLFSECSYGFRPNRSCEMAVRQLLIYLNEGYEWIVDIDLERFFDNVPQDKLMSLVHNIINDGDTESLIRKYLKAGVMTPDGYEETKLGTPQGGNLSPLLSNIMLNELDKELEARGLRFTRYADDCVIAVRSESSAKRVMRTVSDWIQRKLGLKVNTTKTHITRPQKLKYLGFGFYKDSKAKEWKCRPHQESVKKLKRRLKELTCRKMPGTVTGRIAKINQVTRGWINYYALGSMKTAMTEIDAHLRTRLRIIIWKQWKVPKKRQWGLQKLGVDKDLARLTAYCGDRYYWVATKTCVVRAISKDVFAKAGLISCLDYYNERHALKLC

SEQ ID NO: 562 - protein, 427 aa, unidentified
  REGION 1..427: note = group II intron reverse transcriptase
MSELLEKILSKENMNTAYKRVCANKGAGGVDGVTVEELGDYIKEHWRSIREQIRQRQYKPQPVRRVEIPKQNGGVRELGIPTVMDRVIQQGIVQVINPMCEPLFSEWSYGFRPNRSCEMAIRQLLIYLNEGYEWIVDIDLEKFFDNVPQDRLMSLVHDIINDGDTESLIRKYLKAGVMTPQGYEETRLGTPQGGNLSPLLSNIMLNELDKELEARGLRFTRYADDCVIAVKSESSAKRVMRTVSDWIQRKLGLKVNMTKTHITRPLKLKYLGFGFYKDSKTKEWKCRAHQDSIVKLKRKLKELTCRKTPGKVREKIEKINQVTRGWINYYALGSMKTAMTAIDAHLRTRLRVIIWKQWKVPKKRQWGLQKLGVGKDLARLTSYCGDRYQWVVTKTCVVRAISKEVLAKAGLISCLEYYNKRHALKLC

SEQ ID NO: 563 - protein, 427 aa, unidentified
  REGION 1..427: note = group II intron reverse transcriptase
MSELLEKILSKNNMNAAYKKVCANKGAGGVDNVTVEELGDYIKENWESIREQIRRREYKPQPVRRVEIPKPDGGVRKLGIPTVMDRVIQQGIVQVISPMCEPVFSEWSYGFRPNRSCEMAVRQLLVYLNEGYEWIVDIDLERFFDNVPQDRLMSLVHDIINDGDTESLIRKYLKAGVMSRQGYEETKLGTPQGGNLSPLLSNIMLNELDKELEARGLRFTRYADDCVIVVRSESSAKRVMRTVADWIQRKLGLKVNMTKTHITRPQKLKYLGFGFYRDSRAKEWKCRPHQDSVKKFKRKLKELTCRKMPGTVTGRIVQINQVTRGWINYYALGSMKTAMTEIDAHLRTRLRIIIWKQWKVPKKRQWGLQKLGIGKDLARLTAYCGDRYYWVATKTCVVRAISKDVFARSGLISCLDYYNERHALKLC

SEQ ID NO: 564 - protein, 427 aa, unidentified
  REGION 1..427: note = group II intron reverse transcriptase
MSELLEKILSKKNMNAAYKKICANKGAGGVDDVTVEELGDYIKENWESIREQIRRREYKPQPVRRVEIPKPDGGVRKLGIPTVMDRVIQQGIVQIISPMCEPLFSEWSYGFRPNRSCEMAVRQLLMYLNEGYEWIVDIDLEKFFDNVPQDRLMSLVHDIINDGDTESLIRKYLKAGVMTPQGYKETKLGTPQGGNLSPLLSNIMLNELDKELEARGLRFTRYADDCVIAVRSESSAKRVMRTVTDWIQRKLGLKVNMTKTHITRPQKLKYLGFGFYKDSKAKEWKCRPHQDSVKKFKRKLKELTCRKMPGTVTGKIVKINQVTRGWVNYYALGSMKTAMTEIDAHLRTRLRIIIWKQWKAPKKRQWGLQKLGIDKDLARLTAYCGDRYYWVATKTCVNRAISKKVLTKAGLVSCLDYYNERHALKLC

SEQ ID NO: 565 - protein, 427 aa, unidentified
  REGION 1..427: note = group II intron reverse transcriptase
MSELLEKILSQNNMNAAYKKVCANKGAGGVDNVTVEELGDYIKENWESIREQIRRREYKPQPVRRVEIPKPDGGVRKLGIPTVMDRVIQQGIVQVISPMCEPMFSEWSYGFRPNRSCEMAIRQLLVYLNEGYEWIVDIDLERFFDNVPQDRLMSLVHDIINDGDTESLIRKYLKAGVMSRQGYEETKLGTPQGGNLSPLLSNIMLNELDKELGARGLRFTRYADDCVIAVSSESSAKRVMRTVADWIQRKLGLKVNMTKTHITRPQKLKYLGFGFYKDSREKQWKCRPHQDSVKKFKRKLKELTCRKMPGTVTGKIVQINQVTRGWINYYALGSMKTAMTEIDAHLRTRLRIIIWKQWKVPKKRQWGLQKLGIGKDLARLTAYCGDRYYWVATKTCVVRAISKDVFAKAGLISCLDYYNEHHALKLC

SEQ ID NO: 566 - protein, 449 aa, unidentified
  REGION 1..449: note = group II intron reverse transcriptase
MSRYCNHLGQIHENGKEKAHKAMSELLEKVLSKDNMNAAYKRVCANKGAGGVDDVTVEELGDYIKENWEGIREQIRQREYRPQPVRRVEIPKPNGGVRKLGIPTVMDRVIQQGIVQIISPMCEPLFSERSYGFRPDRSCEMAVRQLLVYLNEGYEWIVDIDLEKFFDNVPQDRLMSLVHDIIKDGDTESLIRKYLKAGVMTPQGYEETNLGTPQGGNLSPLLSNIMLNELDKELEERGLRFTRYADDCVIAVKSEASAKRVMRTVTGWIQRKLGLKVNMTKTRITRPQRLKYLGFGLYKDSKAKEWKCRPHQESVKKFKGRLKELTCRKMPGTVTGRIAEINQVTRGWINYYALGSMKTAMTETDAHLRTRLRIIIWKQWKVPKKRQWGLQKLGVSKDLARLTAYCGDRYYWVATKTCVVRAISKDVFAKAGLVSCLDYYNERHALKLC

SEQ ID NO: 567 - protein, 427 aa, unidentified
  REGION 1..427: note = group II intron reverse transcriptase
MSELLEKILSKNNMNAAYKKICANKGAGGVDDVTVEELGDYIKENWESIREQIRRREYKPQPVRRVEIPKSDGGVRKLGIPTVMDRVIQQGIVQIISPMCEPLFSEWSYGFRPNRSCEMAVRQLLMYLNEGYEWIVDIDLEKFFDNVPQDRLMSLVHDIINDGDTESLIRKYLKAGVMTPQGYKETKFGTPQGGNLSPLLSNIMLNELDKELEARGLRFTRYADDCVIAVRSESSAKRVMRTVTDWIQRKLGLKVNMTKTHITRPQKLKYLGFGFYKDSKAKEWKCRPHQDSVKKFKRKLKELTCRKMPGTVTGKIVKINQVTRGWVNYYALGSMKTAMTEIDAHLRTRLRIIIWKQWKAPKKRQWGLQKLGIDKDLARLTAYCGDRYYWVATKTCVNRAISKKVLTKAGLVSCLDYYNERHALKLC

SEQ ID NO: 568 - protein, 427 aa, synthetic construct
MSELLEKILSKRNMNTAYKRVCANKGAGGVDDVTVEELGAYVKENWESIREQIRRREYTPQPVRRVEIPKPRGGERKLGIPTVMDRVIQQGIVQIISPMCEPLFSKWSYGFRPNRSCEMAIRQLLEYLNEGYEWIVDIDLEKFFDNVPQDRLMSLVHDIIKDGDTESLIRKYLKAGAMTPQGYKETKLGTPQGGRLSPLLSNIMLNELDKELEARGLRFTRYADDCVIAVKSESSAKRVMRTVTDWIQRKLGLKVNMTKSQITRPQKLKYLGFGFHKDSKAKEWKCRPHQESVKKFKVRLKELTCRKKPGTVTSKIAKINQVTRGWINYYALGSMKTAMTEIDAHLRTRLRVIIWKQWKVPKKRQWGLQKLGVSKDLARLTAYCGDSYYWVATKTCVVRAISKEVFAKSGLVSCLDYYNERHALKLC

SEQ ID NO: 569 - protein, 427 aa, synthetic construct
MSELLEKILSKRNMNTAYKRVCARKGAGGVDDVTVEELGAYVKENWESIREQIRRREYTPQPVRRVEIPKPRGGERKLGIPTVMDRVIQQGIVQIISPMCEPLFSKWSYGFRPKRSCEMAIRQLLEYLNEGYEWIVDIDLEKFFDNVPQDRLMSLVHDIIKDGDTESLIRKYLKAGAMTPQGYKETKLGTPQGGNLSPLLSNIMLNELDKELEARGLRFTRYADDCVIAVKSESSAKRVMRTVTDWIQRKLGLKVNMTKSQITRPQKLKYLGFGFHKDSKAKEWKCRPHQESVKKFKVRLKELTCRKKPGTVTSKIAKINQVTRGWINYYALGSMKTAMTEIDAHLRTRLRVIIWKQWKVPKKRQWGLQKLGVSKDLARLTAYCGDSYYWVATKTCVVRAISKEVFAKSGLVSCLDYYNERHALKLC

SEQ ID NO: 570 - protein, 427 aa, synthetic construct
MSELLEKILSKRNMNTAYKRVCARKGAGGVDDVTVEELGAYVKENWESIREQIRRREYTPQPVRRVEIPKPRGGERKLGIPTVMDRVIQQGIVQIISPMCEPLFSKWSYGFRPNRSCEMAIRQLLEYLNEGYEWIVDIDLEKFFDNVPQDRLMSLVHDIIKDGDTESLIRKYLKAGAMTPQGYKETKLGTPQGGRLSPLLSNIMLNELDKELEARGLRFTRYADDCVIAVKSESSAKRVMRTVTDWIQRKLGLKVNMTKSQITRPQKLKYLGFGFHKDSKAKEWKCRPHQESVKKFKVRLKELTCRKKPGTVTSKIAKINQVTRGWINYYALGSMKTAMTEIDAHLRTRLRVIIWKQWKVPKKRQWGLQKLGVSKDLARLTAYCGDSYYWVATKTCVVRAISKEVFAKSGLVSCLDYYNERHALKLC

SEQ ID NO: 571 - protein, 427 aa, synthetic construct
MSELLEKILSKRNMNTAYKRVCANKGAGGVDDVTVEELGAYVKENWESIREQIRRREYTPQPVRRVEIPKPRGGERKLGIPTVMDRVIQQGIVQIISPMCEPLFSKWSYGFRPKRSCEMAIRQLLEYLNEGYEWIVDIDLEKFFDNVPQDRLMSLVHDIIKDGDTESLIRKYLKAGAMTPQGYKETKLGTPQGGRLSPLLSNIMLNELDKELEARGLRFTRYADDCVIAVKSESSAKRVMRTVTDWIQRKLGLKVNMTKSQITRPQKLKYLGFGFHKDSKAKEWKCRPHQESVKKFKVRLKELTCRKKPGTVTSKIAKINQVTRGWINYYALGSMKTAMTEIDAHLRTRLRVIIWKQWKVPKKRQWGLQKLGVSKDLARLTAYCGDSYYWVATKTCVVRAISKEVFAKSGLVSCLDYYNERHALKLC

SEQ ID NO: 572 - protein, 427 aa, synthetic construct
MSELLEKILSKRNMNTAYKRVCARKGAGGVDDVTVEELGAYVKENWESIREQIRRREYTPQPVRRVEIPKPRGGERKLGIPTVMDRVIQQGIVQIISPMCEPLFSKWSYGFRPKRSCEMAIRQLLEYLNEGYEWIVDIDLEKFFDNVPQDRLMSLVHDIIKDGDTESLIRKYLKAGAMTPQGYKETKLGTPQGGRLSPLLSNIMLNELDKELEARGLRFTRYADDCVIAVKSESSAKRVMRTVTDWIQRKLGLKVNMTKSQITRPQKLKYLGFGFHKDSKAKEWKCRPHQESVKKFKVRLKELTCRKKPGTVTSKIAKINQVTRGWINYYALGSMKTAMTEIDAHLRTRLRVIIWKQWKVPKKRQWGLQKLGVSKDLARLTAYCGDSYYWVATKTCVVRAISKEVFAKSGLVSCLDYYNERHALKLC

SEQ ID NO: 573 - protein, 427 aa, synthetic construct
MSELLERILSNRNMNAAYKRVCANKGAGGVDGVTVEELGDYIKENWSSIREQIRRRQYKPQPVRRVEIPKPRGGVRNLGIPTVMDRVIQQGIVQVLSPMCEPLFSERSYGFRPNRSCEMAIRQLLVYLNEGCEWIVDIDLEKFFDNVPQDRLMSLVHDIINDGDTESLIRKYLKAGVMTPQGYEETKLGTPQGGRLSPLLSNIMLNELDKELEKRGLRFTRYADDCVIAVKSESSAKRVMRTVADWIQRKLGLKVNMTKTHITRPTKLKYLGFGFYRDNKAKEWKCRPHQDSVAKFKRKLKELTCRKTPGTVTGRIAKINQVTRGWINYYAIGSMKTTMIEIDAHLRTRLRVIIWKQWKVPKKRQWGLQKLGIGKDLARLTSYYGDRYQWIVTKTCVARAISKEVLAKAGLVSCLDYYNERHALKLC

SEQ ID NO: 574 - protein, 427 aa, synthetic construct
MSELLERILSNRNMNAAYKRVCARKGAGGVDGVTVEELGDYIKENWSSIREQIRRRQYKPQPVRRVEIPKPRGGVRNLGIPTVMDRVIQQGIVQVLSPMCEPLFSERSYGFRPKRSCEMAIRQLLVYLNEGCEWIVDIDLEKFFDNVPQDRLMSLVHDIINDGDTESLIRKYLKAGVMTPQGYEETKLGTPQGGNLSPLLSNIMLNELDKELEKRGLRFTRYADDCVIAVKSESSAKRVMRTVADWIQRKLGLKVNMTKTHITRPTKLKYLGFGFYRDNKAKEWKCRPHQDSVAKFKRKLKELTCRKTPGTVTGRIAKINQVTRGWINYYAIGSMKTTMIEIDAHLRTRLRVIIWKQWKVPKKRQWGLQKLGIGKDLARLTSYYGDRYQWIVTKTCVARAISKEVLAKAGLVSCLDYYNERHALKLC

SEQ ID NO: 575 - protein, 427 aa, synthetic construct
MSELLERILSNRNMNAAYKRVCARKGAGGVDGVTVEELGDYIKENWSSIREQIRRRQYKPQPVRRVEIPKPRGGVRNLGIPTVMDRVIQQGIVQVLSPMCEPLFSERSYGFRPNRSCEMAIRQLLVYLNEGCEWIVDIDLEKFFDNVPQDRLMSLVHDIINDGDTESLIRKYLKAGVMTPQGYEETKLGTPQGGRLSPLLSNIMLNELDKELEKRGLRFTRYADDCVIAVKSESSAKRVMRTVADWIQRKLGLKVNMTKTHITRPTKLKYLGFGFYRDNKAKEWKCRPHQDSVAKFKRKLKELTCRKTPGTVTGRIAKINQVTRGWINYYAIGSMKTTMIEIDAHLRTRLRVIIWKQWKVPKKRQWGLQKLGIGKDLARLTSYYGDRYQWIVTKTCVARAISKEVLAKAGLVSCLDYYNERHALKLC

SEQ ID NO: 576 - protein, 427 aa, synthetic construct
MSELLERILSNRNMNAAYKRVCANKGAGGVDGVTVEELGDYIKENWSSIREQIRRRQYKPQPVRRVEIPKPRGGVRNLGIPTVMDRVIQQGIVQVLSPMCEPLFSERSYGFRPKRSCEMAIRQLLVYLNEGCEWIVDIDLEKFFDNVPQDRLMSLVHDIINDGDTESLIRKYLKAGVMTPQGYEETKLGTPQGGRLSPLLSNIMLNELDKELEKRGLRFTRYADDCVIAVKSESSAKRVMRTVADWIQRKLGLKVNMTKTHITRPTKLKYLGFGFYRDNKAKEWKCRPHQDSVAKFKRKLKELTCRKTPGTVTGRIAKINQVTRGWINYYAIGSMKTTMIEIDAHLRTRLRVIIWKQWKVPKKRQWGLQKLGIGKDLARLTSYYGDRYQWIVTKTCVARAISKEVLAKAGLVSCLDYYNERHALKLC

SEQ ID NO: 577 - protein, 427 aa, synthetic construct
MSELLERILSNRNMNAAYKRVCARKGAGGVDGVTVEELGDYIKENWSSIREQIRRRQYKPQPVRRVEIPKPRGGVRNLGIPTVMDRVIQQGIVQVLSPMCEPLFSERSYGFRPKRSCEMAIRQLLVYLNEGCEWIVDIDLEKFFDNVPQDRLMSLVHDIINDGDTESLIRKYLKAGVMTPQGYEETKLGTPQGGRLSPLLSNIMLNELDKELEKRGLRFTRYADDCVIAVKSESSAKRVMRTVADWIQRKLGLKVNMTKTHITRPTKLKYLGFGFYRDNKAKEWKCRPHQDSVAKFKRKLKELTCRKTPGTVTGRIAKINQVTRGWINYYAIGSMKTTMIEIDAHLRTRLRVIIWKQWKVPKKRQWGLQKLGIGKDLARLTSYYGDRYQWIVTKTCVARAISKEVLAKAGLVSCLDYYNERHALKLC

SEQ ID NO: 578 - protein, 698 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 579 - protein, 704 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 580 - protein, 36 aa, synthetic construct
SDLYIERGGDPRDVHQQVETKPKGKRKSEIRILKIR

SEQ ID NO: 581 - protein, 31 aa, synthetic construct
SDYIVDHGGDPEKVFFETKSKKDKTKRYKRR